(19) United States
(12) Patent Application Publication
(10) Pub. No.: US 2026/0139086 A1
(43) Pub. Date: May 21, 2026

(54) ONIUM SALT TYPE MONOMER, MONOMERIC PHOTO-ACID GENERATOR, POLYMER, CHEMICALLY AMPLIFIED RESIST COMPOSITION, AND PATTERNING PROCESS

(71) Applicant: SHIN-ETSU CHEMICAL CO., LTD., Tokyo (JP)

(72) Inventor: Masahiro FUKUSHIMA, Joetsu-shi (JP)

(73) Assignee: SHIN-ETSU CHEMICAL CO., LTD., Tokyo (JP)

(21) Appl. No.: 19/300,792

(22) Filed: Aug. 15, 2025

(30) Foreign Application Priority Data
Aug. 28, 2024 (JP) 2024-146940

(51) Int. Cl. C08F 220/22; C08F 212/14; C08F 212/32; C08F 220/18; G03F 7/00; G03F 7/004; G03F 7/039

(52) U.S. Cl. CPC C08F 220/22; C08F 212/24; C08F 212/32; C08F 220/1808; G03F 7/0045; G03F 7/0397; G03F 7/70033

(57) ABSTRACT

The present invention is onium salt type monomer, wherein the onium salt type monomer is represented by the following general formula (A). This provides: an onium salt type monomer used as a monomeric photo-acid generator, which is a material for a polymer contained in a chemically amplified resist composition that has excellent solvent solubility, high sensitivity, high contrast, and excellent lithography performance in photolithography using high-energy beams; a monomeric photo-acid generator consisting of the onium salt type monomer; a polymer containing a repeating unit derived from the monomeric photo-acid generator; a chemically amplified resist composition containing a base polymer that contains the polymer; and a patterning process using the chemically amplified resist composition.

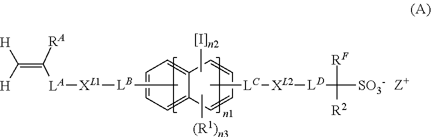

### TECHNICAL FIELD

The present invention relates to: an onium salt type monomer, a monomeric photo-acid generator, a polymer, a chemically amplified resist composition, and a patterning process.

### BACKGROUND ART

As LSIs become more highly integrated and faster, pattern rules are becoming finer at a rapid pace. In particular, the expansion of the flash memory market and the increase in memory capacity are driving miniaturization. For cutting-edge miniaturization technology, 65-nm node devices are being mass-produced using ArF lithography, and preparations are underway for mass production of 45-nm node devices using next-generation ArF immersion lithography. The following candidates for next-generation 32-nm node devices are being considered: immersion lithography using an ultra-high-NA lens that combines a liquid with a higher refractive index than water, a high-refractive-index lens, and a high-refractive-index resist film; extreme ultraviolet (EUV) lithography with a wavelength of 13.5 nm; and double exposure (double patterning lithography) with ArF lithography.

As miniaturization progresses and the diffraction limit of light is approached, the contrast of light is decreasing. The reduction in optical contrast causes a decrease in the resolution of hole and trench patterns and in focus margin in positive resist films.

As patterns become finer, line width roughness (LWR) of line patterns and critical dimension uniformity (CDU) of hole patterns are becoming issues. Possible causes include uneven distribution and aggregation of base polymers and acid generators, and acid diffusion. Furthermore, as resist films become thinner, the LWR tends to increase, and the deterioration of the LWR due to the thinner resist films accompanying the progress of miniaturization is becoming a serious problem.

Resist compositions for EUV lithography needs simultaneously to achieve high sensitivity, high resolution, and low LWR. Reducing the acid diffusion distance reduces the LWR but also reduces the sensitivity. For example, lowering the post-exposure bake (PEB) temperature reduces the LWR but also reduces the sensitivity. Increasing the amount of quencher added also reduces the LWR but it also reduces the sensitivity. It is necessary to break the trade-off between sensitivity and LWR.

In order to suppress acid diffusion, a resist compound containing a repeating unit derived from an onium salt of a sulfonic acid having a polymerizable unsaturated bond has been proposed (Patent Document 1). Such so-called polymer-bound acid generators generate polymeric sulfonic acid upon exposure, and are therefore characterized by an extremely short acid diffusion distance. Furthermore, the sensitivity can be improved by increasing the ratio of the acid generator. In the case of additive-type acid generators, increasing the amount added increases the sensitivity, but in this case, the acid diffusion distance also increases. Since acid diffuses ununiformly, increased acid diffusion leads to degradation of LWR and CDU. It can be said that polymeric acid generators have high capabilities in terms of the balance between sensitivity, LWR, and CDU.

Iodine atoms have a very high absorption rate for EUV light with a wavelength of 13.5 nm, and it has been confirmed that secondary electrons are generated from iodine atoms during exposure, making this technology attractive for EUV lithography. Patent Document 2 describes a photo-acid generator in which an iodine atom has been introduced into the anion, and Patent Document 3 describes a polymerizable-group-containing photo-acid generator in which an iodine atom has been introduced into the anion. Although this has been confirmed to improve lithography performance to a certain extent, iodine atoms are not highly soluble in organic solvents, and there is a concern that they may precipitate in the solvent.

Patent Documents 4 and 5 describe a photo-acid generator wherein a pentafluorosulfanyl group (—SF₅ group) and a trifluoromethoxy group (—OCF₃ group) have been introduced into the cation. Although these efforts have led to some progress in improving lithography performance, there is still room for improvement, and there is a need for the development of resist materials that are effective for forming even finer patterns.

### CITATION LIST

### Patent Literature

Patent Document 1: JP4425776B2
Patent Document 2: JP6720926B2
Patent Document 3: JP6973274B2
Patent Document 4: WO2023/223624A1
Patent Document 5: JP2022-059112A

### SUMMARY OF INVENTION

### Technical Problem

There is a demand for the development of acid-catalyzed chemically amplified resist compositions that have higher sensitivity and can improve lithography performance such as exposure latitude (EL), LWR, CDU, and depth of focus (DOF).

The present invention has been made in view of the above circumstances, and has an object of providing: an onium salt type monomer used as a monomeric photo-acid generator, which is a material for a polymer contained in a chemically amplified resist composition that has excellent solvent solubility, high sensitivity, high contrast, and excellent lithography performance in photolithography using high-energy beams; a monomeric photo-acid generator consisting of the onium salt type monomer; a polymer containing a repeating unit derived from the monomeric photo-acid generator; a chemically amplified resist composition containing a base polymer that contains the polymer; and a patterning process using the chemically amplified resist composition.

### Solution to Problem

In order to solve the above problems, the present invention provides an onium salt type monomer, wherein the onium salt type monomer is represented by the following general formula (A),

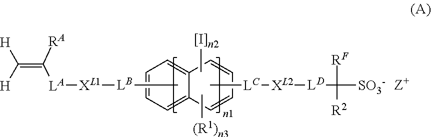

wherein “n1” represents 0 or 1; “n2” represents an integer from 1 to 4; “n3” represents an integer from 0 to 4; provided that when “n1” is 0, 1≤n2+n3≤4, and when “n1” is 1, 1≤n2+n3≤6;
R^{A} represents a hydrogen atom, a fluorine atom, a methyl group, or a trifluoromethyl group;
R¹ represents a halogen atom other than an iodine atom, a nitro group, a hydroxy group, a carboxy group, a hydrocarbyl group having 1 to 20 carbon atoms and optionally containing a heteroatom, a hydrocarbyloxy group having 1 to 20 carbon atoms and optionally containing a heteroatom, a hydrocarbylthio group having 1 to 20 carbon atoms and optionally containing a heteroatom, or a hydrocarbyloxycarbonyl group having 2 to 20 carbon atoms; when “n3” is 2, 3, or 4, each R¹ may be the same as or different from each other, and a plurality of R¹ may be bonded to each other to form a ring together with the carbon atoms to which they are bonded;
R² represents a hydrogen atom, or a hydrocarbyl group having 1 to 20 carbon atoms and optionally containing a heteroatom;
L^{A} represents a single bond, an ether bond, an ester bond, or a sulfonate ester bond;
L^{B}, L^{C}, and L^{D} each independently represent a single bond, an ether bond, an ester bond, a sulfonate ester bond, a carbonate bond, or a carbamate bond;
X^{L1} and X^{L2} each independently represent a single bond, or a hydrocarbylene group having 1 to 40 carbon atoms and optionally containing a heteroatom;
R^{F} represents a fluorine atom or a fluorinated saturated hydrocarbyl group having 1 to 6 carbon atoms; and
Z⁺ represents an onium cation.

Such an onium salt type monomer can be used as a monomeric photo-acid generator, and a chemically amplified resist composition containing a base polymer containing a polymer that contains a repeating unit derived from the monomeric photo-acid generator has excellent solvent solubility, high sensitivity, high contrast, and excellent lithography performance in photolithography using high-energy beams.

In the present invention, the onium salt type monomer is preferably represented by the following general formula (A1),

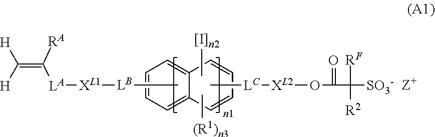

wherein R^{A}, R¹, R², R^{F}, L^{A}, L^{B}, L^{C}, X^{L1}, X^{L2}, “n1” to “n3”, and Z⁺ are as defined above.

The onium salt type monomer of the present invention preferably has such a structure.

In this case, the onium salt type monomer represented by the general formula (A1) is preferably represented by the following general formula (A2),

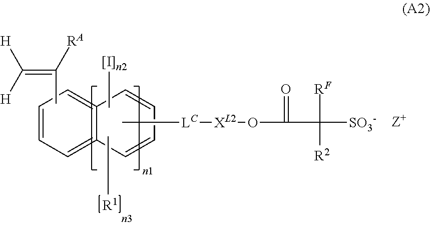

wherein R^{A}, R¹, R², R^{F}, L^{C}, X^{L2}, “n1” to “n3”, and Z⁺ are as defined above.

The onium salt type monomer of the present invention more preferably has such a structure.

Furthermore, in the present invention, the Z⁺ preferably represents a sulfonium cation represented by the following general formula (Z-1) or an iodonium cation represented by the following general formula (Z-2),

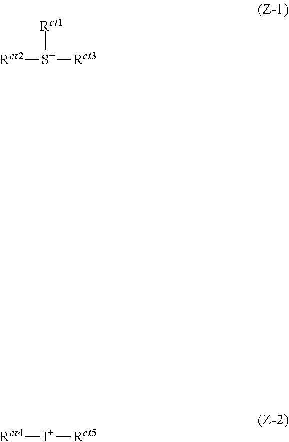

wherein R^{ct1} to R^{ct5} each independently represent a halogen atom, or a hydrocarbyl group having 1 to 30 carbon atoms and optionally containing a heteroatom; and also, R^{ct1} and R^{ct2} may be bonded to each other to form a ring together with the sulfur atom to which they are bonded.

The onium salt type monomer of the present invention preferably has such a cation.

The present invention provides a monomeric photo-acid generator containing the onium salt type monomer described above.

When such a monomeric photo-acid generator is used, a chemically amplified resist composition containing a base polymer that contains a polymer containing a repeating unit derived from this monomer has excellent solvent solubility, high sensitivity, high contrast, and excellent lithography performance in photolithography using high-energy beams.

The present invention provides a polymer that contains a repeating unit derived from the monomeric photo-acid generator described above.

When such a polymer is used, a chemically amplified resist composition containing a base polymer that contains this polymer has excellent solvent solubility, high sensitivity, high contrast, and excellent lithography performance in photolithography using high-energy beams.

In the present invention, it is preferable that the polymer further contains at least one repeating unit selected from the group consisting of a repeating unit represented by the following general formula (a1), a repeating unit represented by the following general formula (a2), and a repeating unit represented by the following general formula (a3),

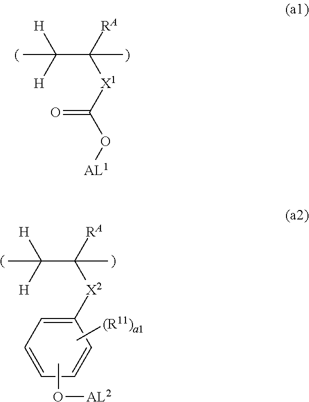

wherein “a1” is an integer from 0 to 4;
R^{A} each independently represents a hydrogen atom, a fluorine atom, a methyl group, or a trifluoromethyl group;
X¹ represents a single bond, a phenylene group, a naphthylene group, or *—C(═O)—O—X¹¹—, and the phenylene group or naphthylene group may be substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom; X¹¹ represents a saturated hydrocarbylene group having 1 to 10 carbon atoms, a phenylene group, or a naphthylene group, and the saturated hydrocarbylene group may contain a hydroxy group, an ether bond, an ester bond, or a lactone ring;
X² represents a single bond or *—C(═O)—O—;
“*” represents an attachment point to a main chain carbon atom;
R²¹ represents a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; when “a1” is 2 or more, each R²¹ may be the same as or different from each other; and
AL¹ and AL² each independently represent an acid-labile group,

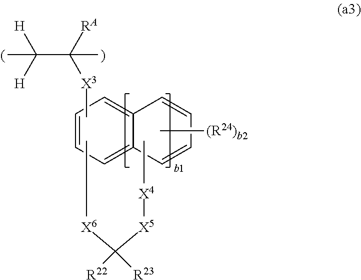

wherein “b1” represents 0 or 1; “b2” represents an integer from 0 to 3 when “b1” is 0, and represents an integer from 0 to 5 when “b1” is 1;
R^{A} represents a hydrogen atom, a fluorine atom, a methyl group, or a trifluoromethyl group;
X³ represents a single bond, *—C(═O)—O—, or *—C(═O)—NH—; “*” represents an attachment point to a main chain carbon atom;
X⁴ represents a single bond, an aliphatic hydrocarbylene group having 1 to 4 carbon atoms, a carbonyl group, a sulfonyl group, or a group obtained by combining these;
X⁵ and X⁶ each independently represent an oxygen atom or a sulfur atom; provided that X⁴ and X⁶ are bonded to adjacent carbon atoms of an aromatic ring;
R²² and R²³ each independently represent a hydrogen atom, or a hydrocarbyl group having 1 to 20 carbon atoms and optionally containing a heteroatom; also, R²² and R²³ may be bonded to each other to form a ring together with the carbon atom to which they are bonded;
R²⁴ represents a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, a hydrocarbylthio group having 1 to 20 carbon atoms and optionally containing a heteroatom, or —N(R^{24A})(R^{24B}); R^{24A} and R^{24B} each independently represent a hydrogen atom or a hydrocarbyl group having 1 to 6 carbon atoms; and when “b2” is 2 or more, each R²⁴ may be the same as or different from each other, and a plurality of R²⁴ may be bonded to each other to form a ring together with the carbon atoms of the aromatic ring to which they are bonded.

In the present invention, it is preferable that the polymer further contains at least one repeating unit selected from the group consisting of a repeating unit represented by the following general formula (b1) and a repeating unit represented by the following general formula (b2),

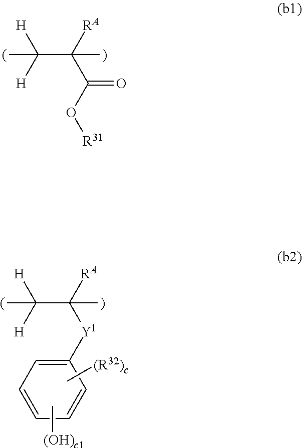

wherein “c1” is an integer from 1 to 4; “c2” represents an integer from 0 to 4; and 1≤c1+c2≤5;
R^{A} each independently represents a hydrogen atom, a fluorine atom, a methyl group, or a trifluoromethyl group;
Y¹ represents a single bond or *—C(═O)—O—; “*” represents an attachment point to a main chain carbon atom;
R³¹ represents a hydrogen atom, or a group having 1 to 20 carbon atoms and containing at least one structure selected from the group consisting of a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—);
R³² represents a halogen atom, a carboxy group, a nitro group, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and when “c2” represents 2 or more, each R³² may be the same as or different from each other.

The polymer of the present invention preferably has such a repeating unit.

The present invention provides a chemically amplified resist composition containing a base polymer that contains the polymer (A) described above.

When such a chemically amplified resist composition is used, excellent solvent solubility, high sensitivity, high contrast, and excellent lithography performance can be achieved in photolithography using high-energy beams.

In this case, it is preferable that the chemically amplified resist composition further contains one or more selected from an organic solvent (B), a quencher (C), a photo-acid generator (D) other than the monomeric photo-acid generator, and a surfactant (E).

The chemically amplified resist composition of the present invention may contain these additives.

The present invention also provides a patterning process, comprising the steps of forming a resist film on a substrate using the chemically amplified resist composition described above, exposing the resist film with a high-energy beam, and developing the exposed resist film using a developer.

When such a patterning process is used, it is possible to provide a patterning process using a chemically amplified resist composition that has excellent solvent solubility, high sensitivity, high contrast, and excellent lithography performance in photolithography using high-energy beams.

In this case, it is preferable to use KrF excimer laser light, ArF excimer laser light, an electron beam, or extreme ultraviolet having a wavelength of 3 to 15 nm as the high-energy beam.

The above-mentioned high-energy beams can be used.

### Advantageous Effects of Invention

As described above, when a pattern is formed using a chemically amplified resist composition containing a base polymer containing a polymer that contains a repeating unit that functions as a photo-acid generator derived from a monomeric photo-acid generator consisting of an onium salt-type monomer of the present invention, it is possible to form a resist pattern that has excellent solvent solubility, high contrast, and good sensitivity, and is excellent in terms of lithography performance such as EL, LWR, CDU, and DOF, particularly in photolithography that uses high-energy beams such as KrF excimer laser light, ArF excimer laser light, an electron beam (EB), and EUV.

### DESCRIPTION OF EMBODIMENTS

As described above, there has been a demand for the development of an onium salt type monomer used as a monomeric photo-acid generator, which is a material for a polymer contained in a chemically amplified resist composition that has excellent solvent solubility, high sensitivity, high contrast, and excellent lithography performance in photolithography using high-energy beams; a monomeric photo-acid generator consisting of the onium salt type monomer; a polymer containing a repeating unit derived from the monomeric photo-acid generator; a chemically amplified resist composition containing a base polymer that contains the polymer; and a patterning process using the chemically amplified resist composition.

As a result of earnest studies to achieve the above object, the present inventor found that by using a polymer containing a repeating unit derived from an onium salt type monomer having a polymerizable group, an iodine-containing aromatic ring, and an α-fluoroalkanesulfonate anion structure as a polymer-bound acid generator, it is possible to obtain a chemically amplified resist composition which has good sensitivity, improved lithography performance such as EL, LWR, CDU, and DOF, high contrast, and high resolution. He thus completed the present invention.

That is, the present invention is an onium salt type monomer, wherein the onium salt type monomer is represented by the following general formula (A),

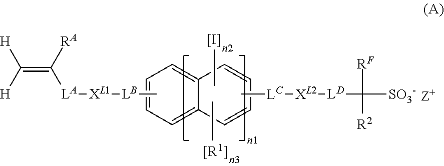

wherein “n1” represents 0 or 1; “n2” represents an integer from 1 to 4; “n3” represents an integer from 0 to 4; provided that when “n1” is 0, 1≤n2+n3≤4, and when “n1” is 1, 1≤n2+n3≤6;
R^{A} represents a hydrogen atom, a fluorine atom, a methyl group, or a trifluoromethyl group;
R¹ represents a halogen atom other than an iodine atom, a nitro group, a hydroxy group, a carboxy group, a hydrocarbyl group having 1 to 20 carbon atoms and optionally containing a heteroatom, a hydrocarbyloxy group having 1 to 20 carbon atoms and optionally containing a heteroatom, a hydrocarbylthio group having 1 to 20 carbon atoms and optionally containing a heteroatom, or a hydrocarbyloxycarbonyl group having 2 to 20 carbon atoms; when “n3” is 2, 3, or 4, each R¹ may be the same as or different from each other, and a plurality of R¹ may be bonded to each other to form a ring together with the carbon atoms to which they are bonded;
R² represents a hydrogen atom, or a hydrocarbyl group having 1 to 20 carbon atoms and optionally containing a heteroatom;
L^{A} represents a single bond, an ether bond, an ester bond, or a sulfonate ester bond;
L^{B}, L^{C}, and L^{D} each independently represent a single bond, an ether bond, an ester bond, a sulfonate ester bond, a carbonate bond, or a carbamate bond;
X^{L1} and X^{L2} each independently represent a single bond, or a hydrocarbylene group having 1 to 40 carbon atoms and optionally containing a heteroatom;
R^{F} represents a fluorine atom or a fluorinated saturated hydrocarbyl group having 1 to 6 carbon atoms; and
Z⁺ represents an onium cation.

Hereinafter, the present invention will be described in detail. In the following explanation, some structures represented by chemical formulae may have asymmetric carbons and may have enantiomers or diastereomers, and in such cases, the isomers will be represented by a single formula. The isomers may be used alone or in a combination of two or more types.

### [Onium Salt Type Monomer]

The onium salt type monomer of the present invention is represented by the following general formula (A).

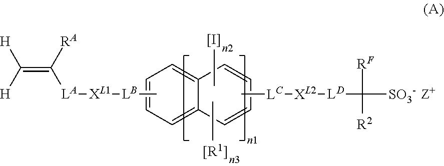

In the formula, “n1” represents 0 or 1; “n2” represents an integer from 1 to 4; “n3” represents an integer from 0 to 4; provided that when “n1” is 0, 1≤n2+n3≤4, and when “n1” is 1, 1≤n2+n3≤6;
R^{A} represents a hydrogen atom, a fluorine atom, a methyl group, or a trifluoromethyl group;
R¹ represents a halogen atom other than an iodine atom, a nitro group, a hydroxy group, a carboxy group, a hydrocarbyl group having 1 to 20 carbon atoms and optionally containing a heteroatom, a hydrocarbyloxy group having 1 to 20 carbon atoms and optionally containing a heteroatom, a hydrocarbylthio group having 1 to 20 carbon atoms and optionally containing a heteroatom, or a hydrocarbyloxycarbonyl group having 2 to 20 carbon atoms; when “n3” is 2, 3, or 4, each R¹ may be the same as or different from each other, and a plurality of R¹ may be bonded to each other to form a ring together with the carbon atoms to which they are bonded;
R² represents a hydrogen atom, or a hydrocarbyl group having 1 to 20 carbon atoms and optionally containing a heteroatom;
L^{A} represents a single bond, an ether bond, an ester bond, or a sulfonate ester bond;
L^{B}, L^{C}, and L^{D} each independently represent a single bond, an ether bond, an ester bond, a sulfonate ester bond, a carbonate bond, or a carbamate bond;
X^{L1} and X^{L2} each independently represent a single bond, or a hydrocarbylene group having 1 to 40 carbon atoms and optionally containing a heteroatom;
R^{F} represents a fluorine atom or a fluorinated saturated hydrocarbyl group having 1 to 6 carbon atoms; and
Z⁺ represents an onium cation.

In the general formula (A), “n1” represents 0 or 1. When “n1” is 0, the structure is a benzene ring, and when “n1” is 1, it is a naphthalene ring; however, from the viewpoint of solvent solubility, it is preferable that “n1” is 0, giving a benzene ring. “n2” represents an integer from 1 to 4. The greater the number of iodine atoms in the anion structure, the greater the absorption, particularly with respect to EUV, but there is a concern regarding precipitation in the resist composition due to poor solubility in solvents, so “n2” is preferably 1, 2, or 3, and further preferably 1 or 2. “n3” represents an integer from 0 to 4. From the viewpoint of raw material procurement, “n3” is preferably 0 or 1.

In the general formula (A), R^{A} represents a hydrogen atom, a fluorine atom, a methyl group, or a trifluoromethyl group. Of these, a hydrogen atom or a methyl group is preferable.

In the general formula (A), R¹ represents a halogen atom other than an iodine atom, a nitro group, a hydroxy group, a carboxy group, a hydrocarbyl group having 1 to 20 carbon atoms and optionally containing a heteroatom, a hydrocarbyloxy group having 1 to 20 carbon atoms and optionally containing a heteroatom, a hydrocarbylthio group having 1 to 20 carbon atoms and optionally containing a heteroatom, or a hydrocarbyloxycarbonyl group having 2 to 20 carbon atoms. Examples of the halogen atom include a fluorine atom, a chlorine atom, and a bromine atom. The hydrocarbyl groups and the hydrocarbyl moieties of the hydrocarbyloxy and the hydrocarbylthio groups may be saturated or unsaturated, and may be linear, branched, or cyclic. Specific examples thereof include alkyl groups having 1 to 20 carbon atoms, such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl groups; cyclic saturated hydrocarbyl groups having 3 to 20 carbon atoms, such as cyclopropyl, cyclopentyl, cyclohexyl, cyclopropylmethyl, 4-methylcyclohexyl, cyclohexylmethyl, norbornyl, and adamantyl groups; alkenyl groups having 2 to 20 carbon atoms, such as vinyl, 1-propenyl, 2-propenyl, butenyl, and hexenyl groups; cyclic unsaturated hydrocarbyl groups having 3 to 20 carbon atoms, such as cyclohexenyl groups; aryl groups having 6 to 20 carbon atoms, such as phenyl and naphthyl groups; aralkyl groups having 7 to 20 carbon atoms, such as benzyl, 1-phenylethyl, and 2-phenylethyl groups; and groups obtained by combining these groups. Of these, aryl groups are preferable. Furthermore, some or all of the hydrogen atoms of the hydrocarbyl groups may be substituted with a group containing a heteroatom such as an oxygen atom, a sulfur atom, a nitrogen atom, or a halogen atom, and some of the —CH₂— of the hydrocarbyl groups may be substituted with a group containing a heteroatom such as an oxygen atom, a sulfur atom, or a nitrogen atom, and as a result, they may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group. When “n3” is 2, 3, or 4, each R¹ may be the same as or different from each other.

Furthermore, when “n3” is 2, 3, or 4, a plurality of R¹ may be bonded to each other to form a ring together with the carbon atoms of the aromatic ring to which they are bonded. Specific examples of the ring formed in this case include a cyclopropane ring, a cyclobutane ring, a cyclopentane ring, a cyclohexane ring, a norbornane ring, and an adamantane ring. Furthermore, some or all of the hydrogen atoms in the ring may be substituted with a group containing a heteroatom such as an oxygen atom, a sulfur atom, a nitrogen atom, or a halogen atom, and some of the —CH₂— in the ring may be substituted with a group containing a heteroatom such as an oxygen atom, a sulfur atom, or a nitrogen atom, and as a result, it may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group.

In the general formula (A), R² represents a hydrogen atom, or a hydrocarbyl group having 1 to 20 carbon atoms and optionally containing a heteroatom. The hydrocarbyl group may be saturated or unsaturated, and may be linear, branched, or cyclic. Specific examples thereof include, but are not limited to, ones similar to R¹. From the viewpoint of raw material procurement, R² preferably represents a hydrogen atom.

In the general formula (A), R^{F} represents a fluorine atom or a fluorinated saturated hydrocarbyl group having 1 to 6 carbon atoms. The fluorinated saturated hydrocarbyl group having 1 to 6 carbon atoms is preferably a trifluoromethyl group.

In the general formula (A), L^{A} represents a single bond, an ether bond, an ester bond, or a sulfonate ester bond. Among these, when both L^{B} and X^{L1} described later are single bonds, L^{A} preferably represents a single bond.

In the general formula (A), L^{B}, L^{C}, and L^{D} each independently represent a single bond, an ether bond, an ester bond, a sulfonate ester bond, a carbonate bond, or a carbamate bond. Of these, L^{B} preferably represents a single bond, an ether bond, an ester bond, or a sulfonate ester bond, and more preferably a single bond, an ether bond, or an ester bond. L^{C} preferably represents a single bond, an ether bond, an ester bond, or a sulfonate ester bond, and more preferably a single bond, an ether bond, or an ester bond. L^{D} preferably represents a single bond, an ether bond, an ester bond, or a sulfonate ester bond, and more preferably an ether bond or an ester bond.

In the general formula (A), X^{L1} and X^{L2} represent a single bond, or a hydrocarbylene group having 1 to 40 carbon atoms and optionally containing a heteroatom. The hydrocarbylene group may be linear, branched, or cyclic, and specific examples thereof include an alkanediyl group and a cyclic saturated hydrocarbylene group. Specific examples of the heteroatom include an oxygen atom, a nitrogen atom, and a sulfur atom.

Specific examples of the hydrocarbylene group having 1 to 40 carbon atoms optionally containing a heteroatom represented by X^{L1} and X^{L2} include, but are not limited to, the following. In the following formulae, “*” is an attachment point to L^{A} and L^{B}, or L^{C} and L^{D} respectively.

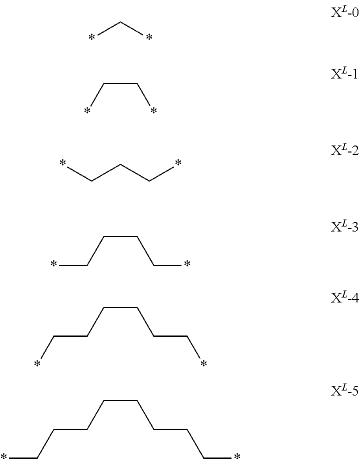

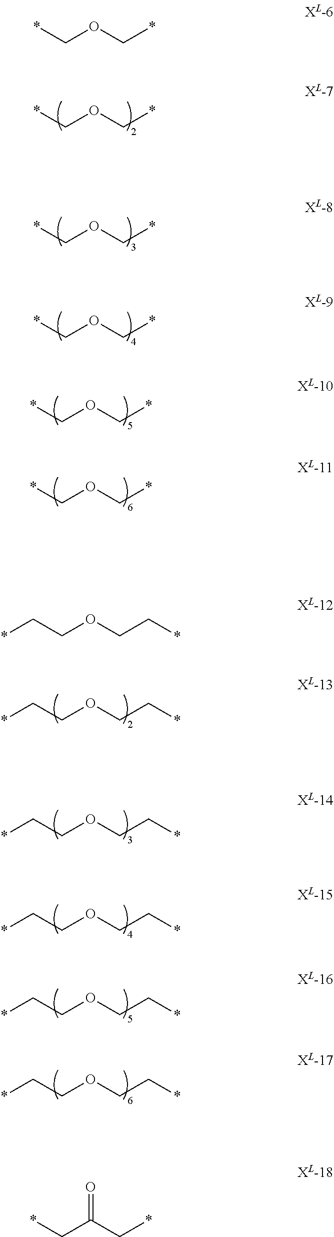

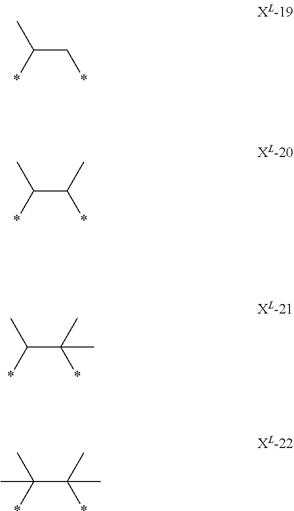

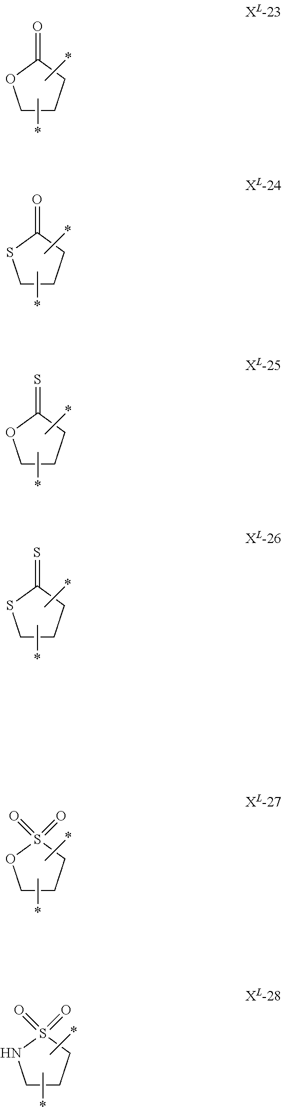

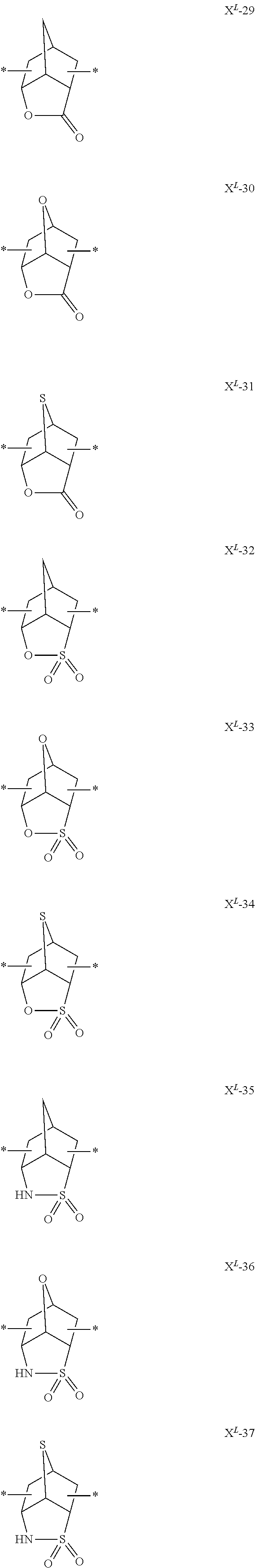

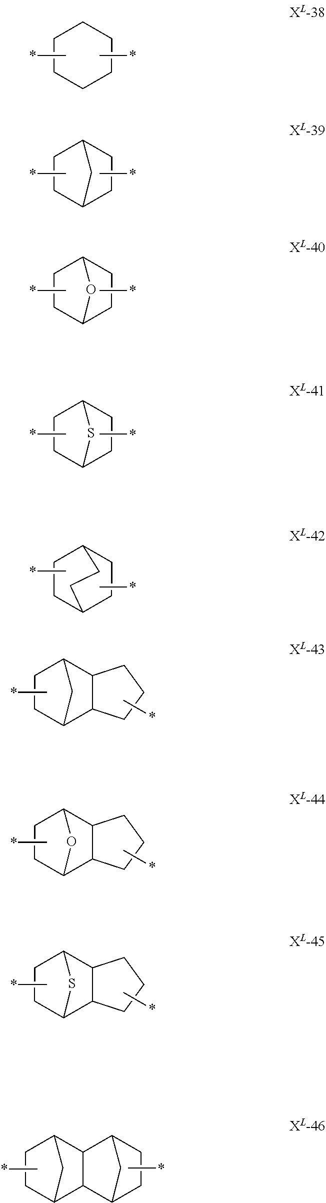

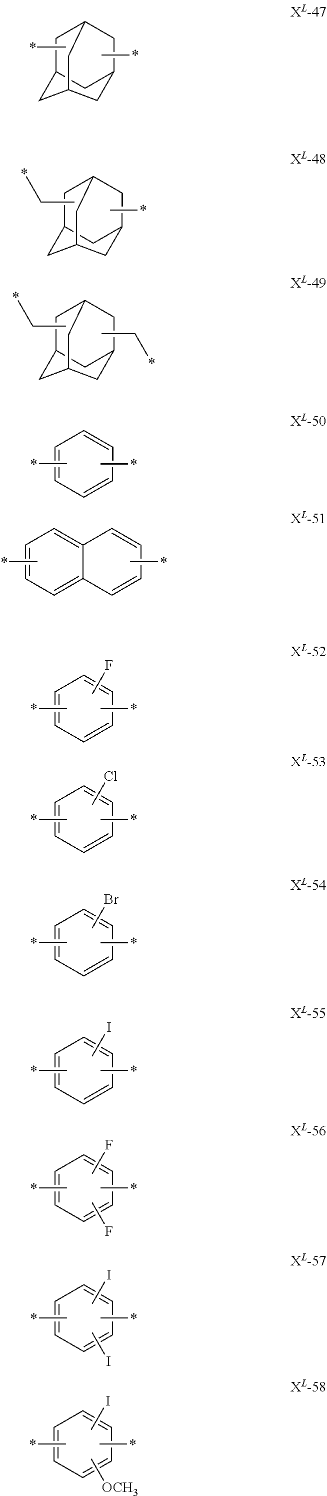

Of these, X^{L}-0 to X^{L}-22, X^{L}-29 to X^{L}-34, and X^{L}-47 to X^{L}-58 are preferable.

From the viewpoint of the rigidity of the obtained polymer, X^{L1} and X^{L2} preferably represent a single bond.

The onium salt type monomer is preferably represented by the following general formula (A1).

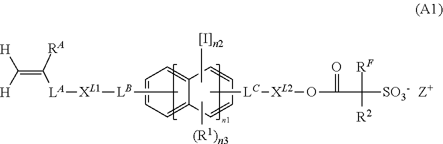

In the formula, R^{A}, R¹, R², R^{F}, L^{A}, L^{B}, L^{C}, X^{L1}, X^{L2}, “n1” to “n3”, and Z⁺ are as defined above.

The onium salt type monomer represented by the general formula (A1) is preferably represented by the following general formula (A2).

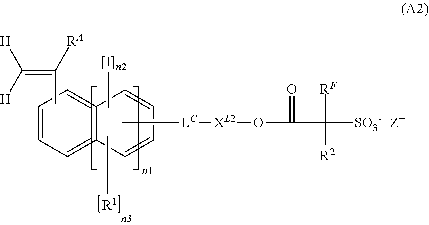

In the formula, R^{A}, R¹, R², R^{F}, L^{C}, X^{L2}, “n1” to “n3”, and Z⁺ are as defined above.

Specific examples of the anion of the onium salt type monomer represented by the general formula (A) include, but are not limited to, the following. In the following formulae, R^{A} is as defined above, and Me represents a methyl group. Furthermore, the bonding positions of the various substituents on the aromatic ring may be interchanged.

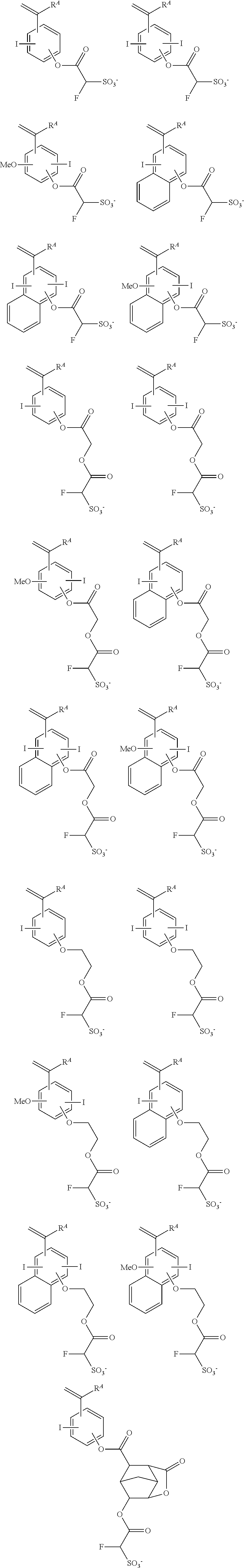

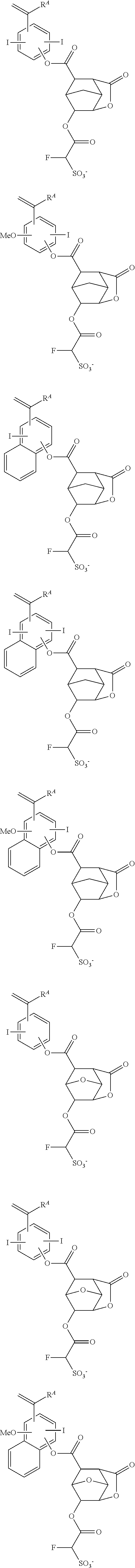

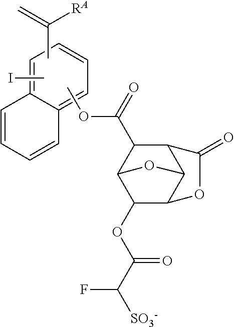

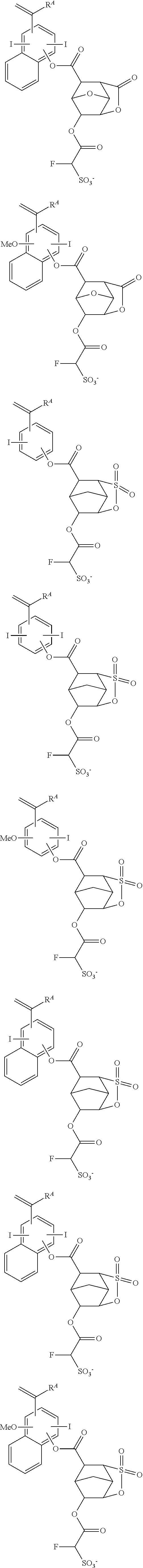

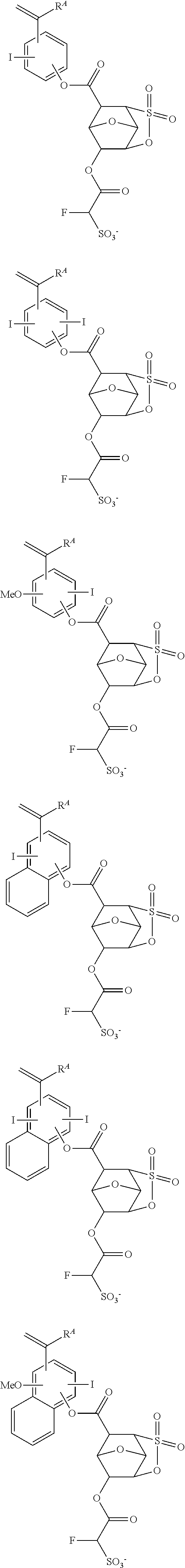

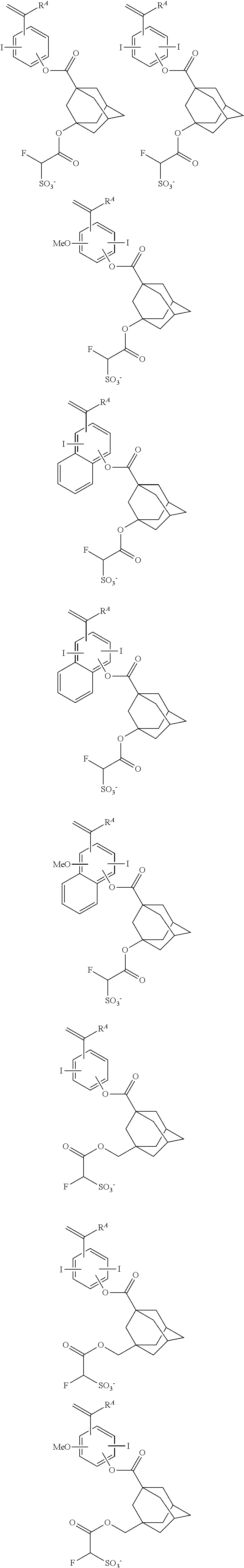

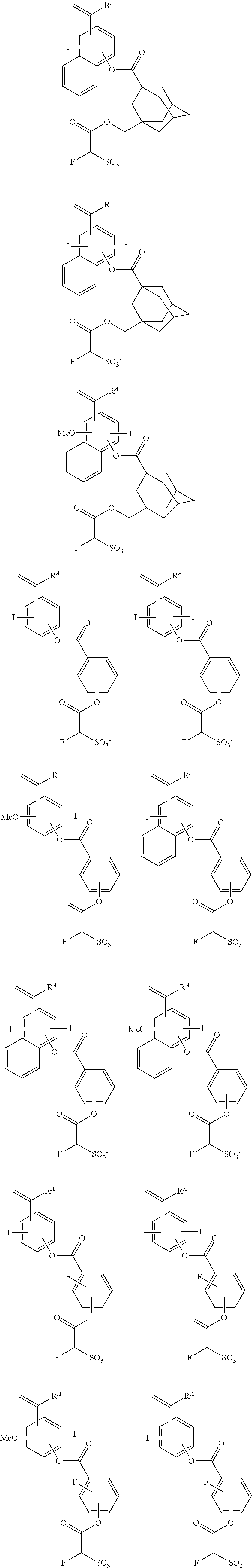

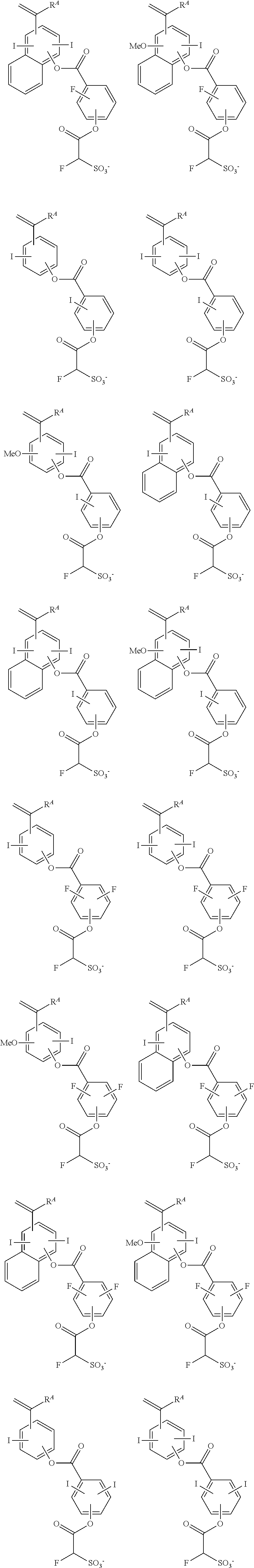

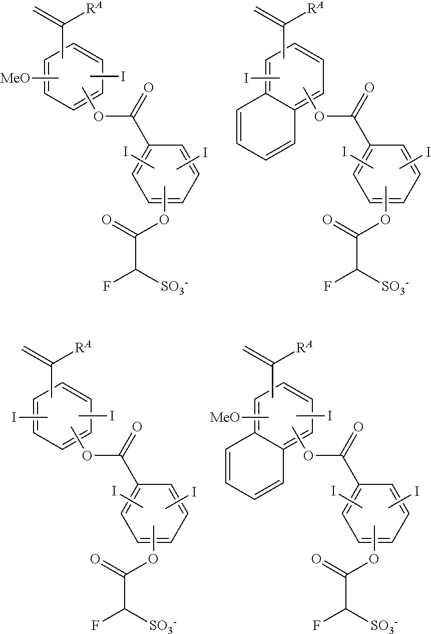

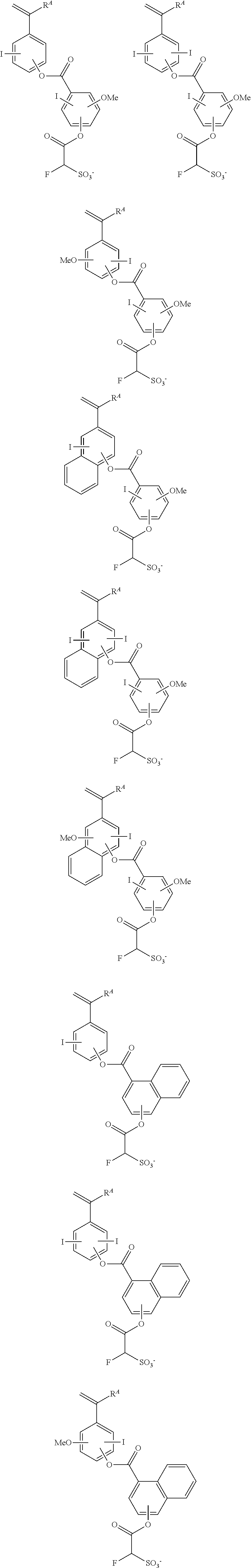

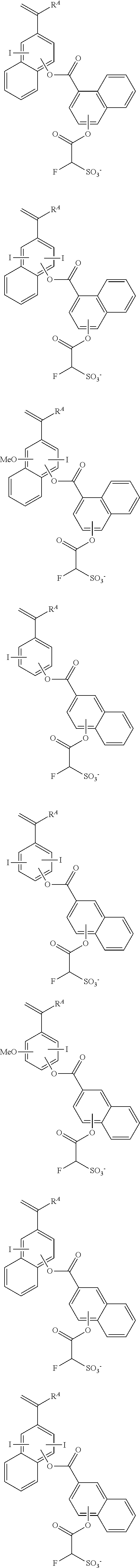

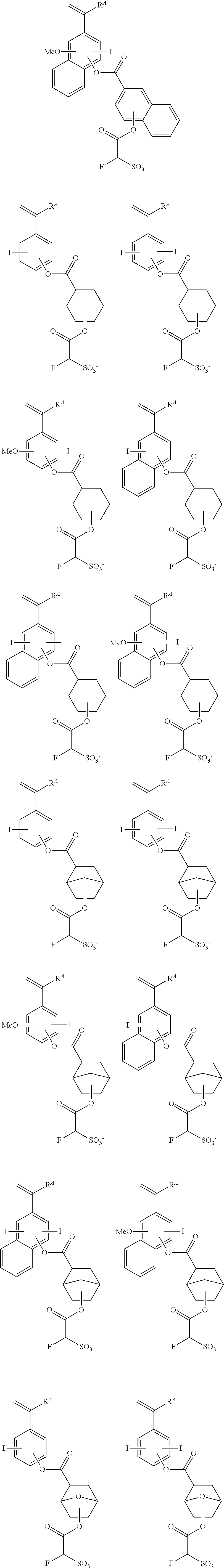

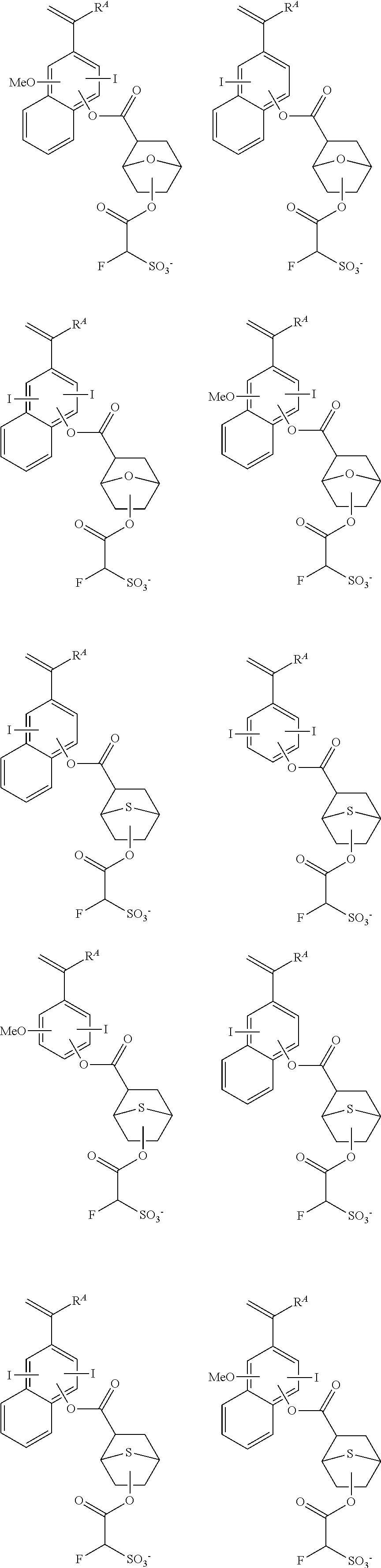

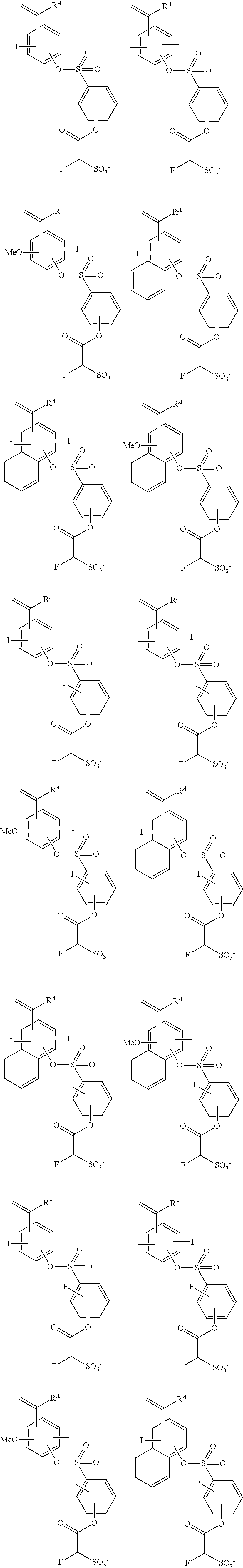

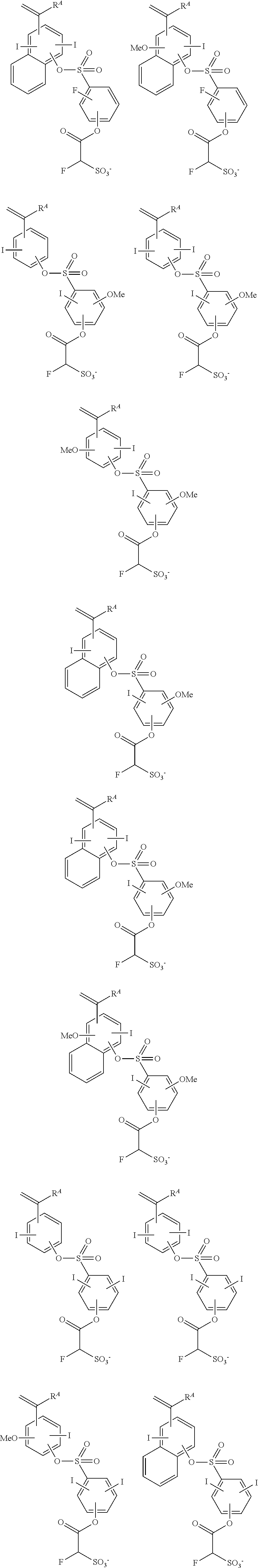

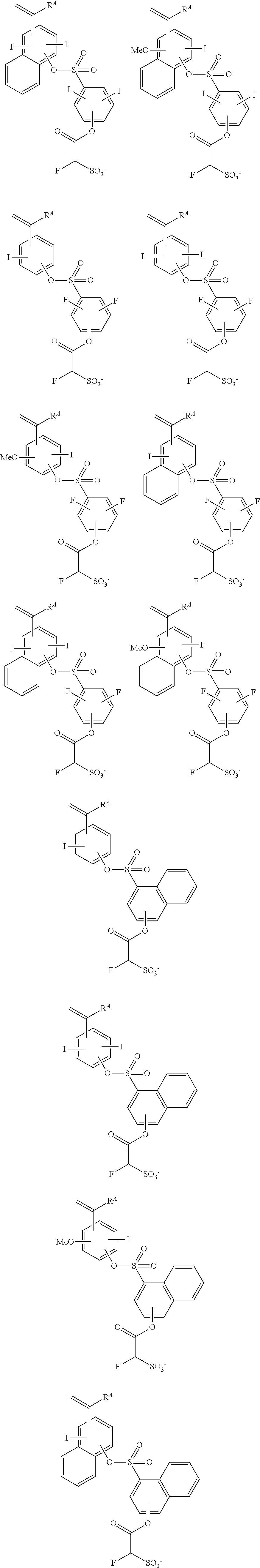

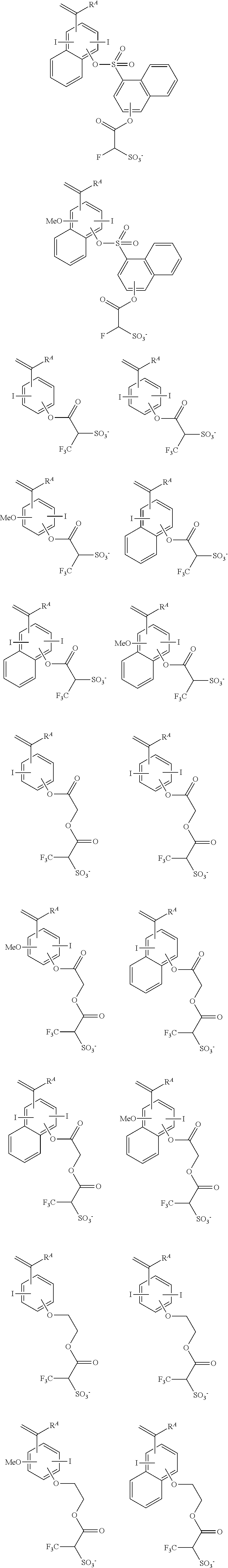

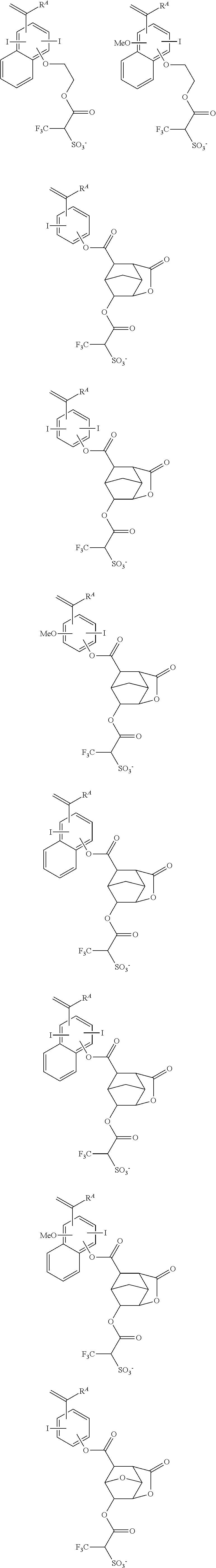

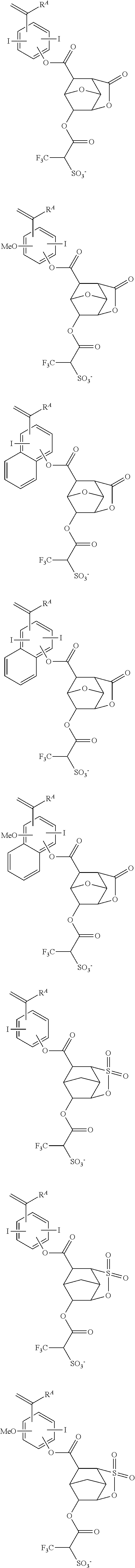

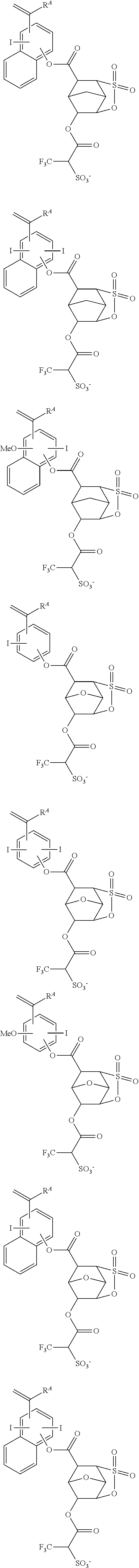

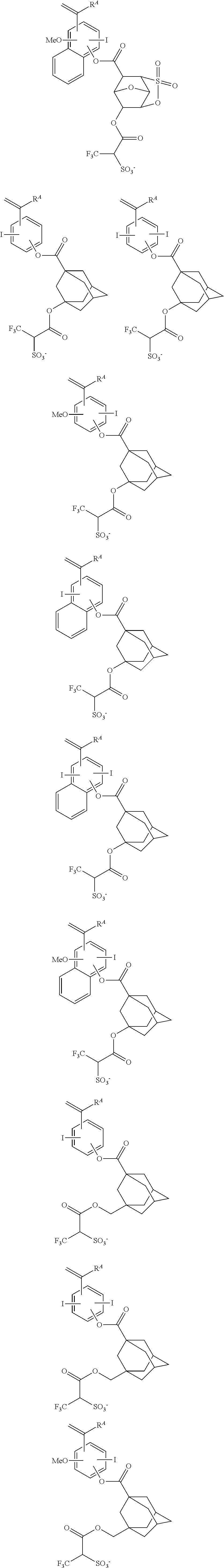

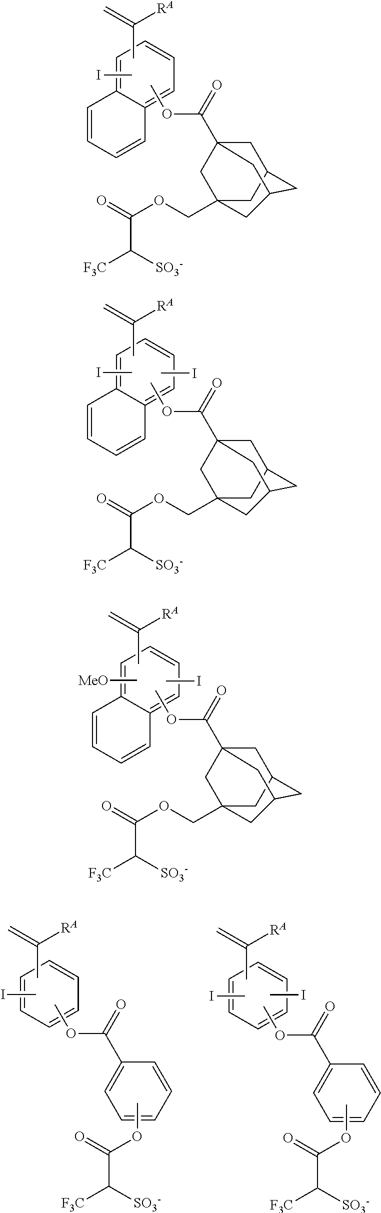

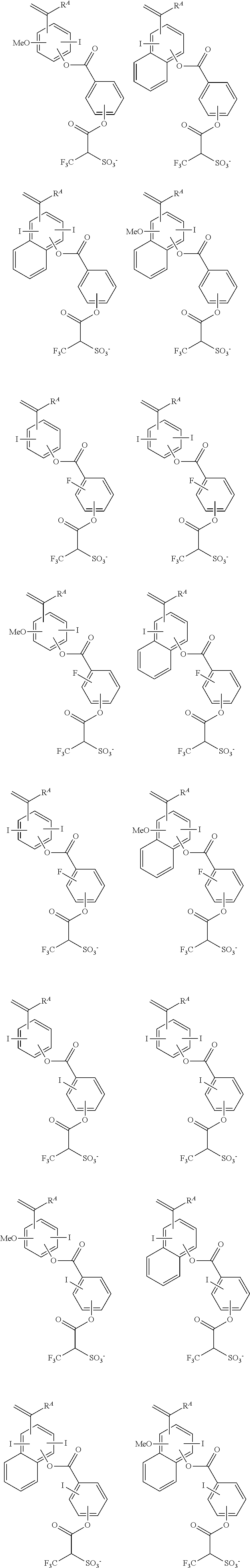

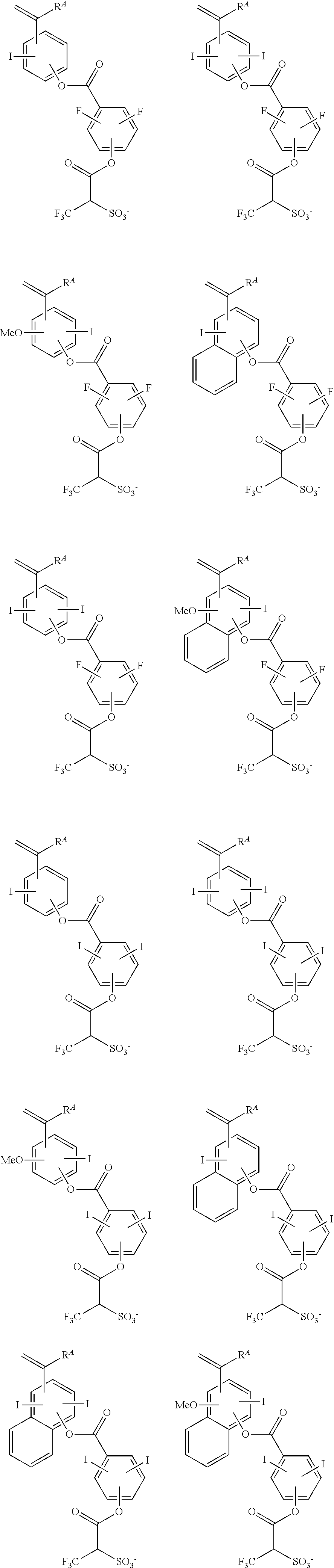

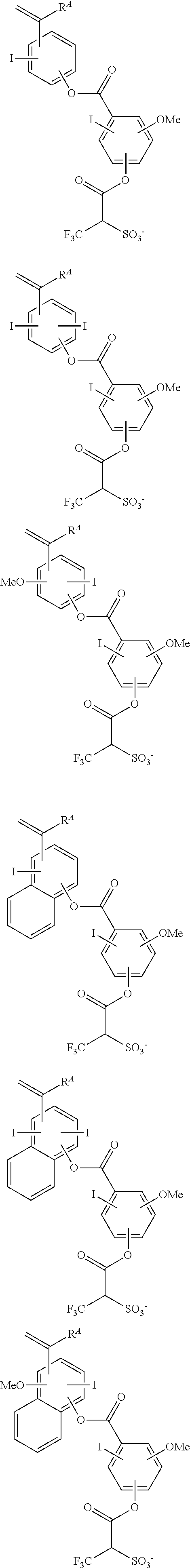

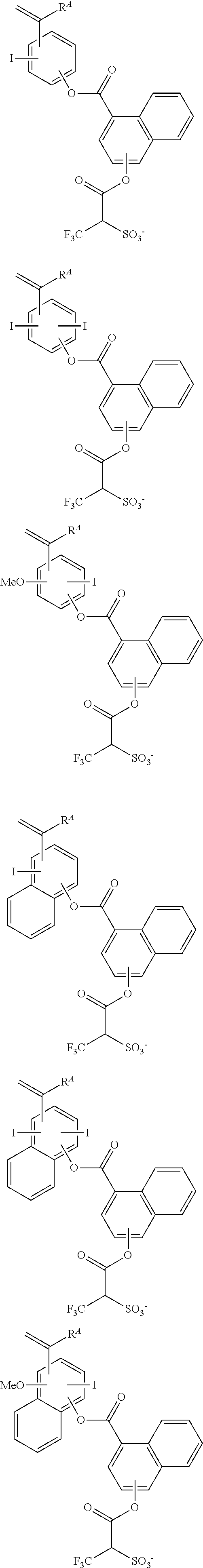

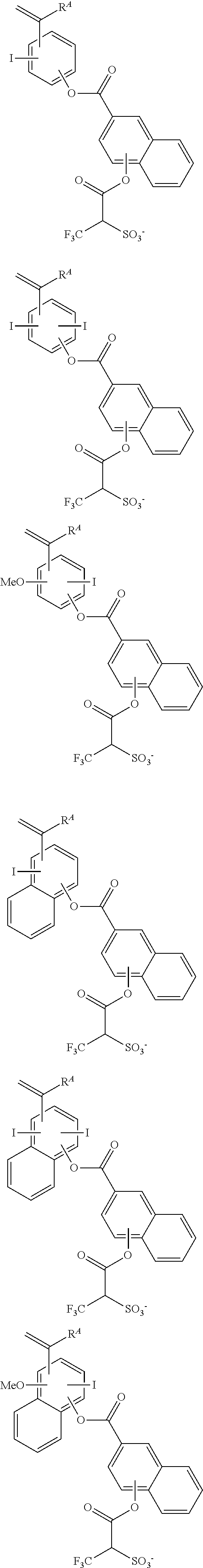

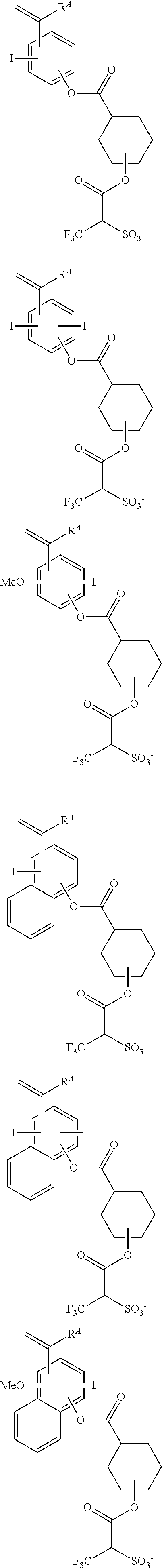

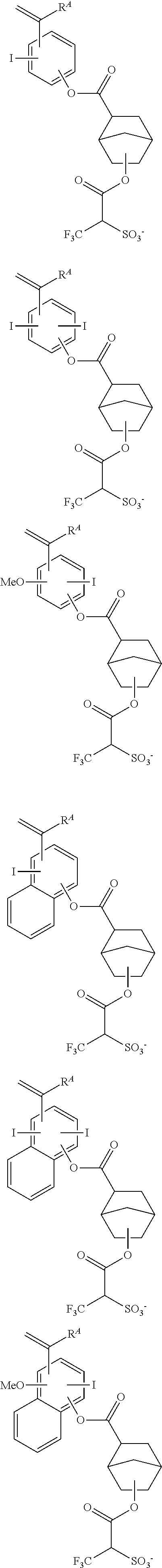

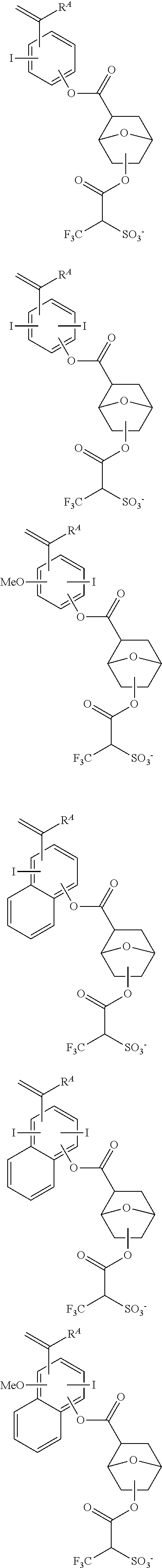

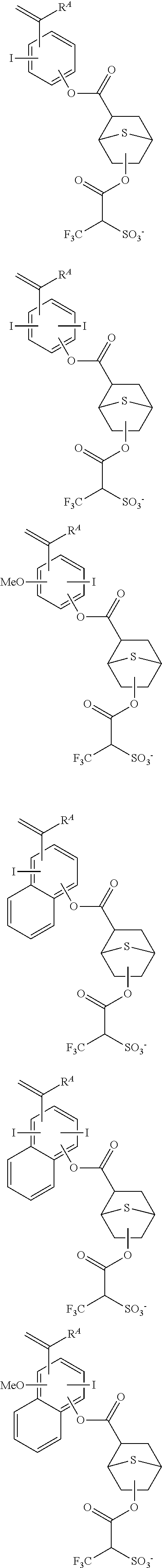

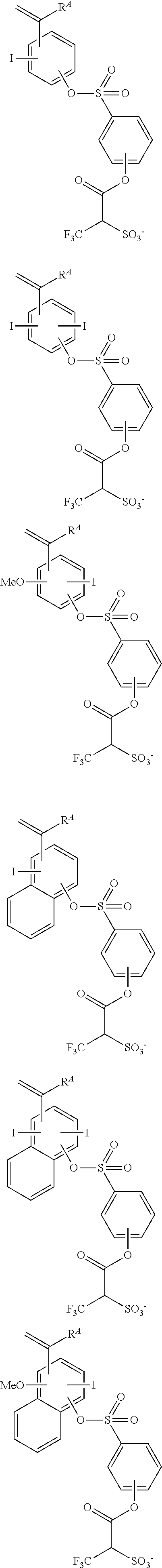

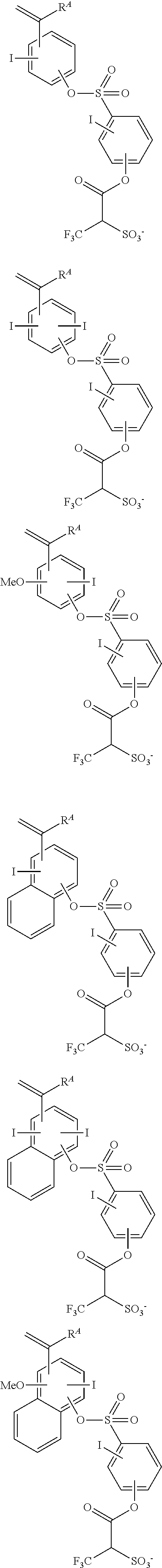

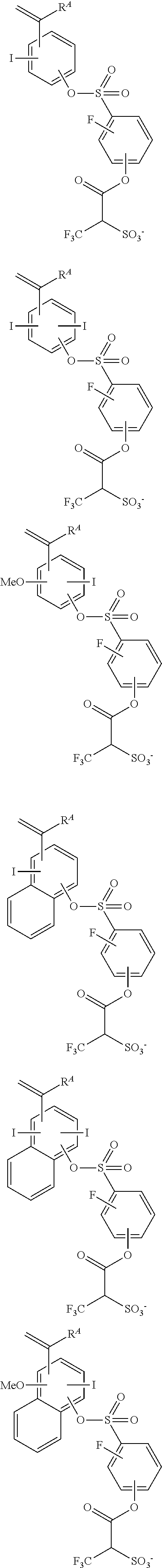

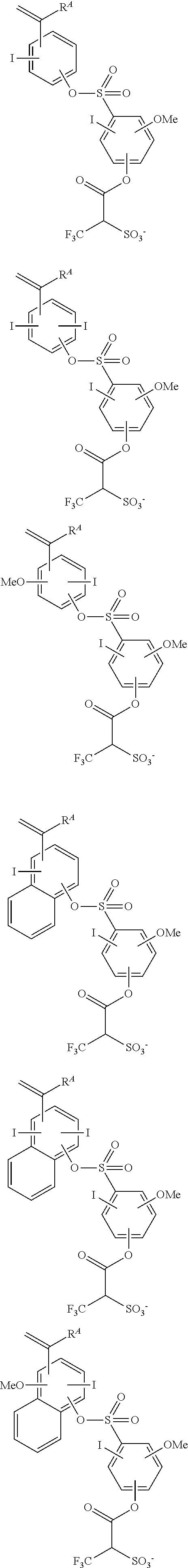

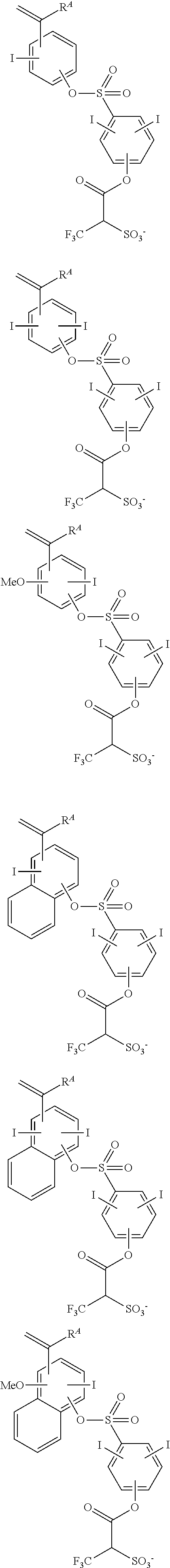

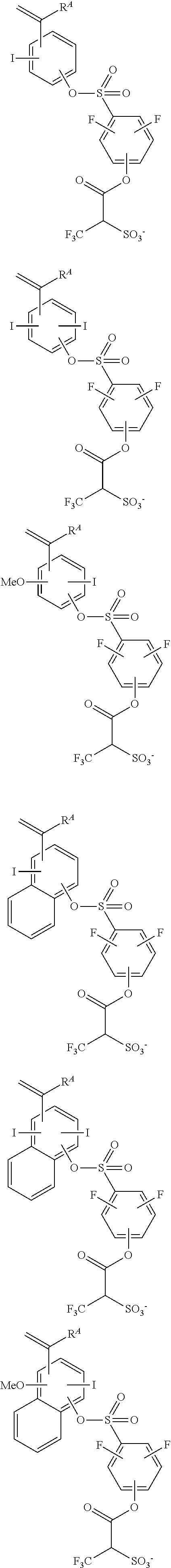

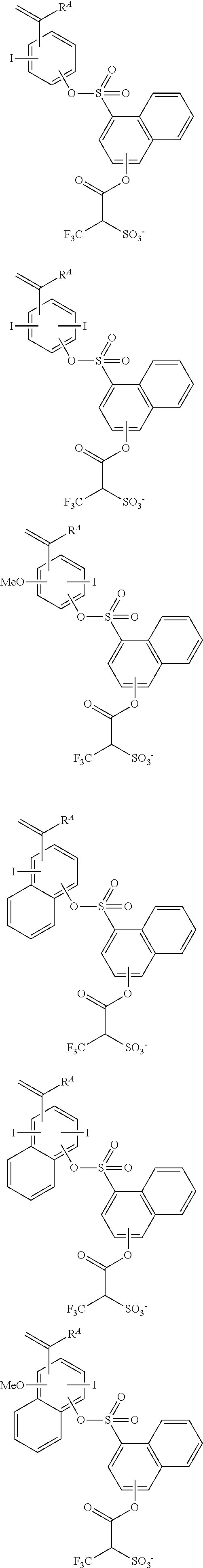

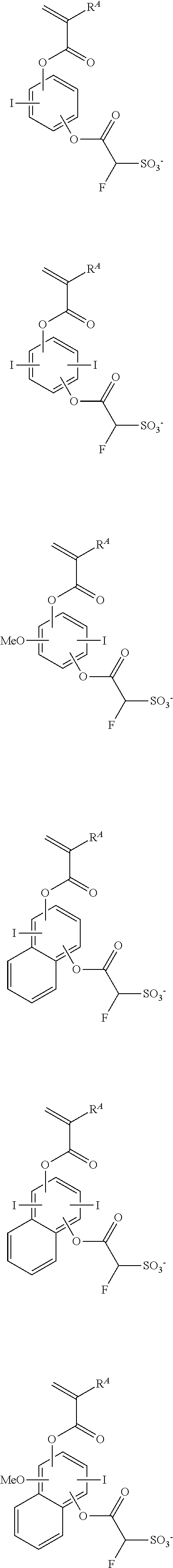

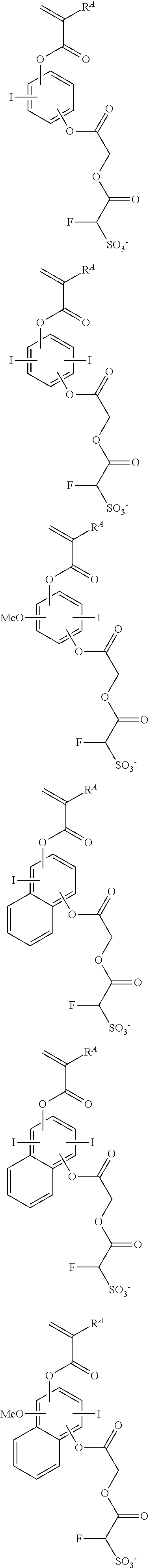

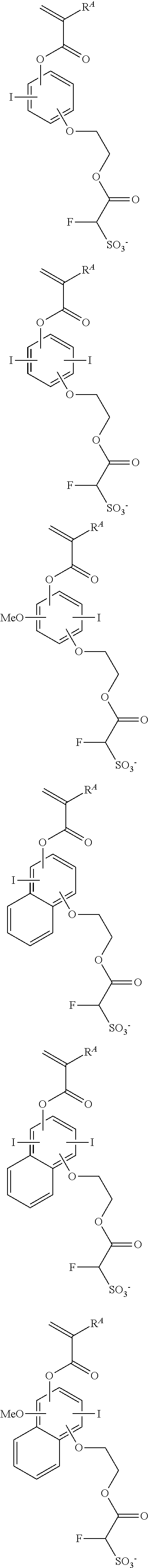

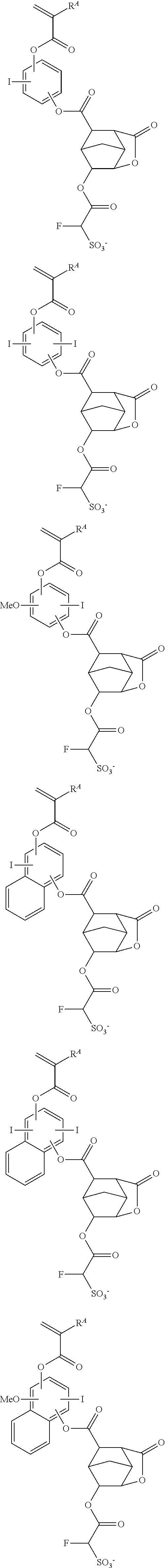

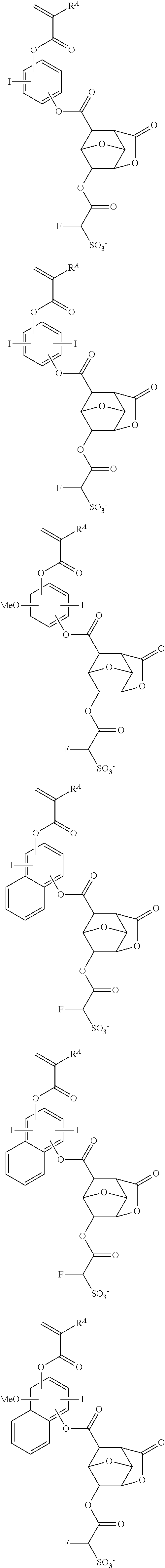

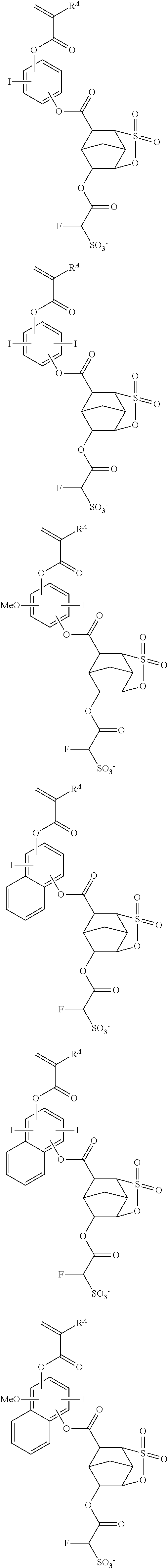

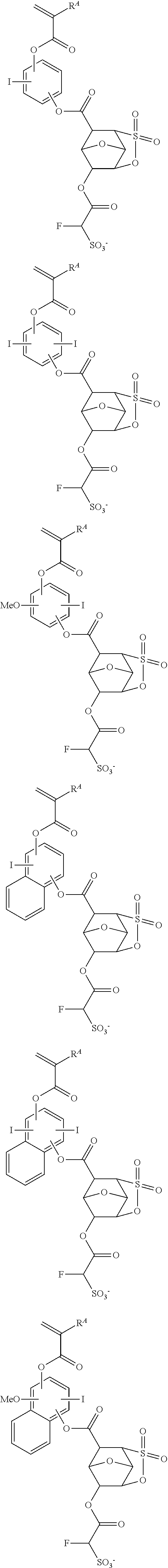

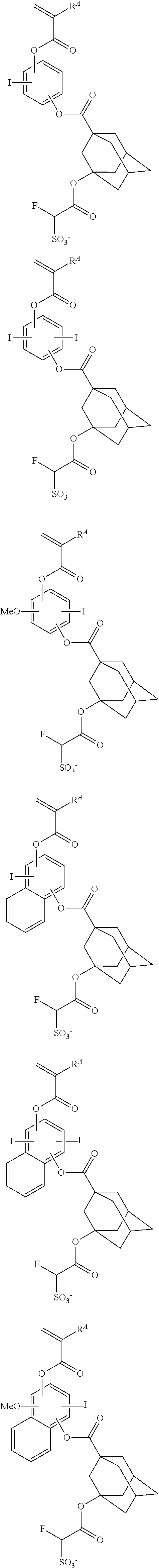

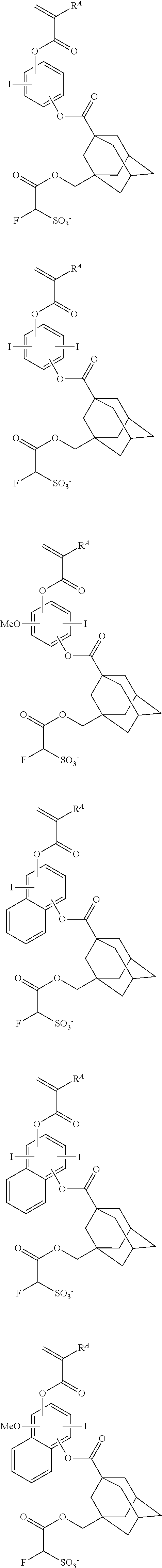

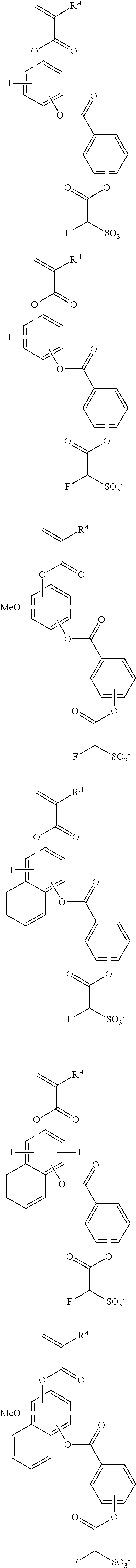

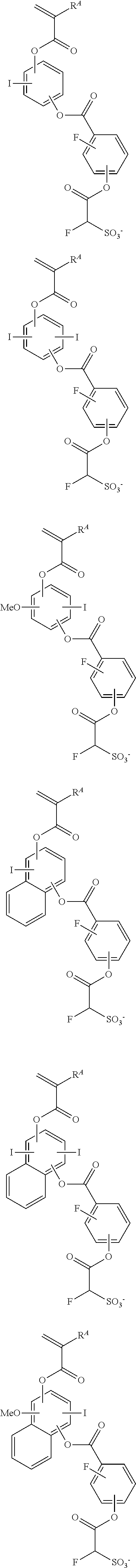

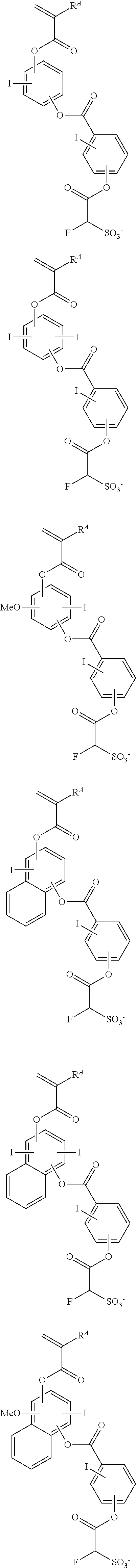

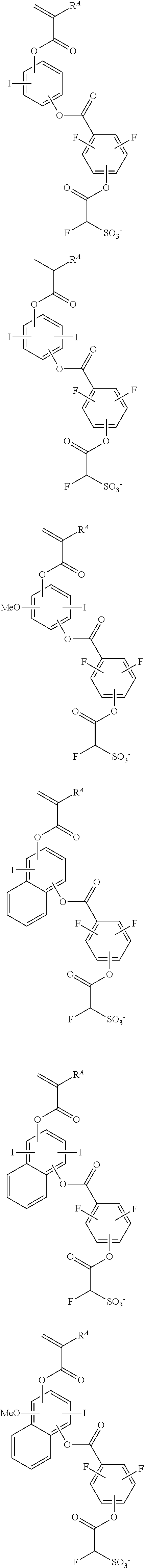

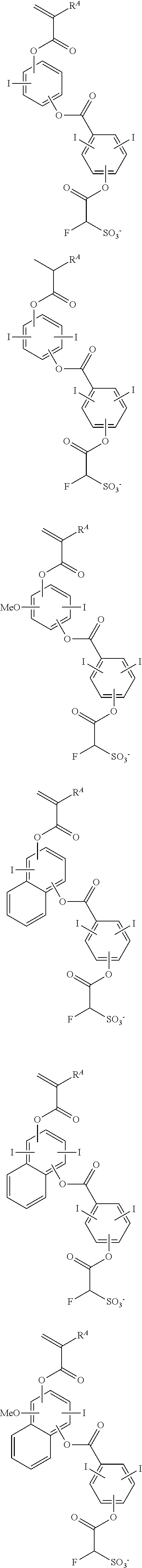

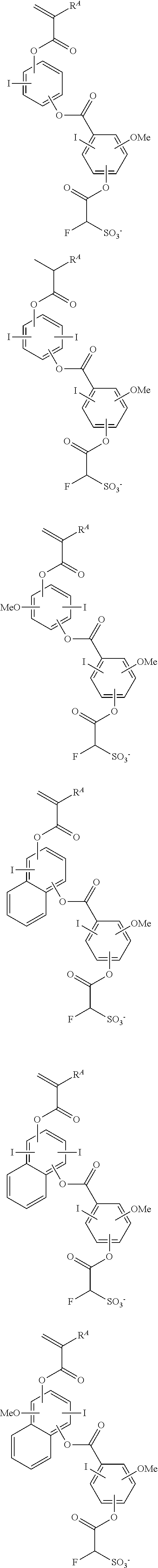

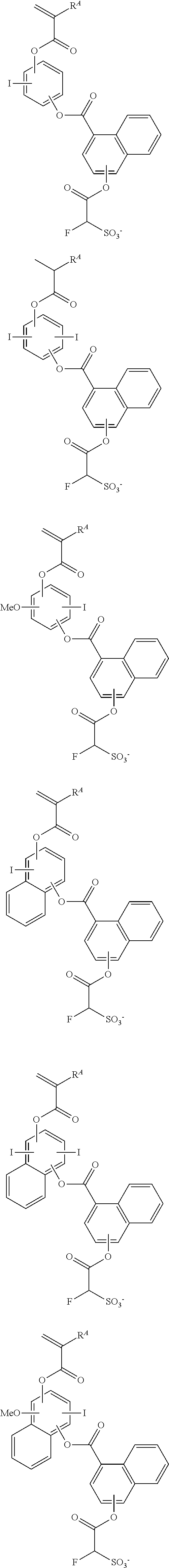

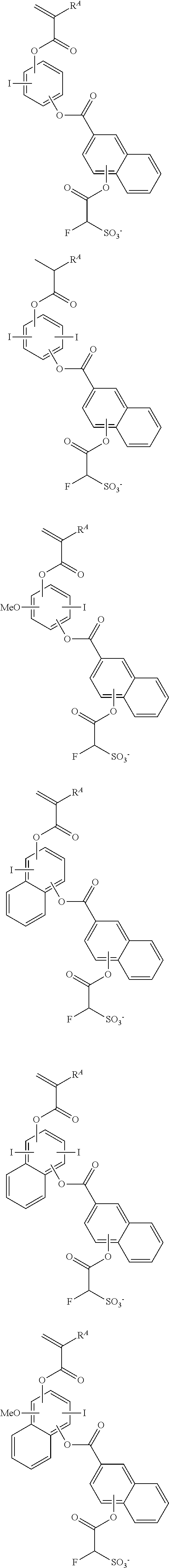

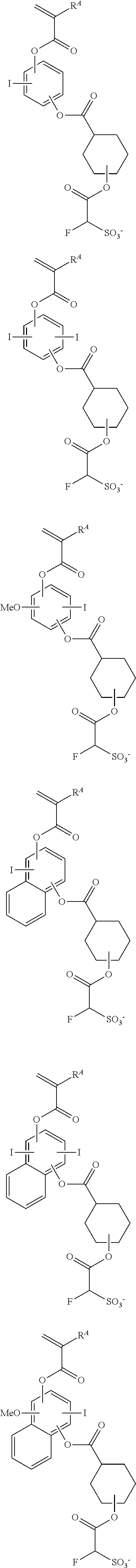

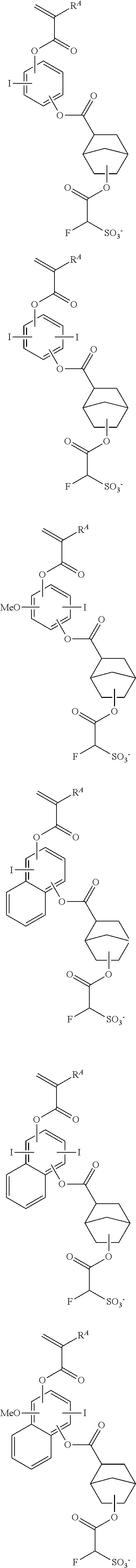

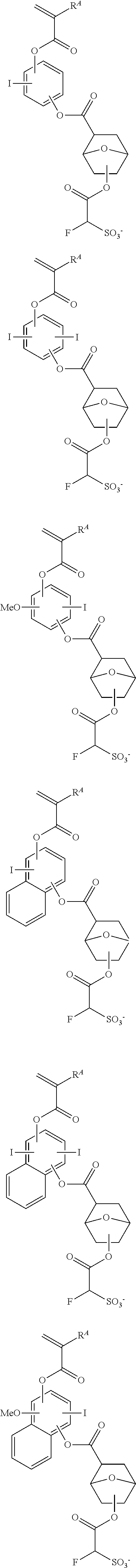

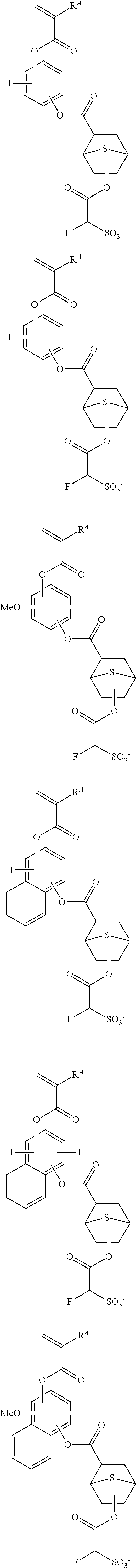

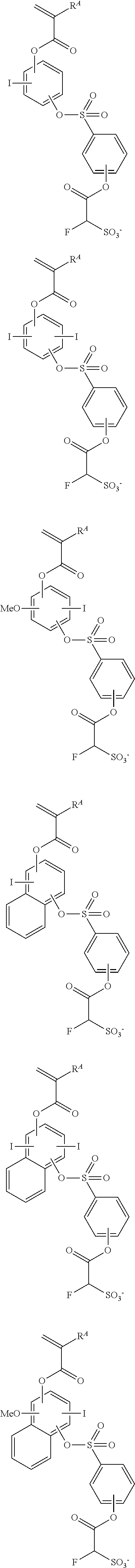

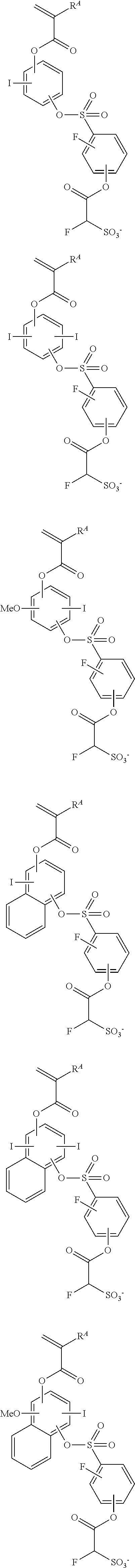

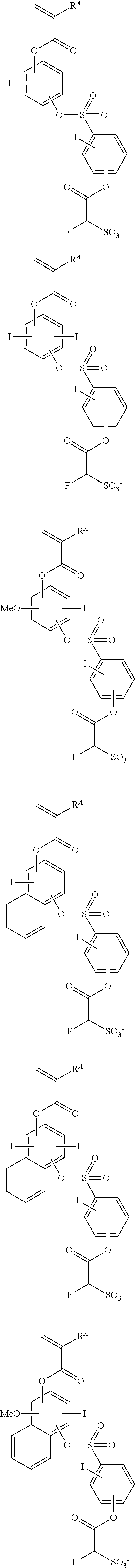

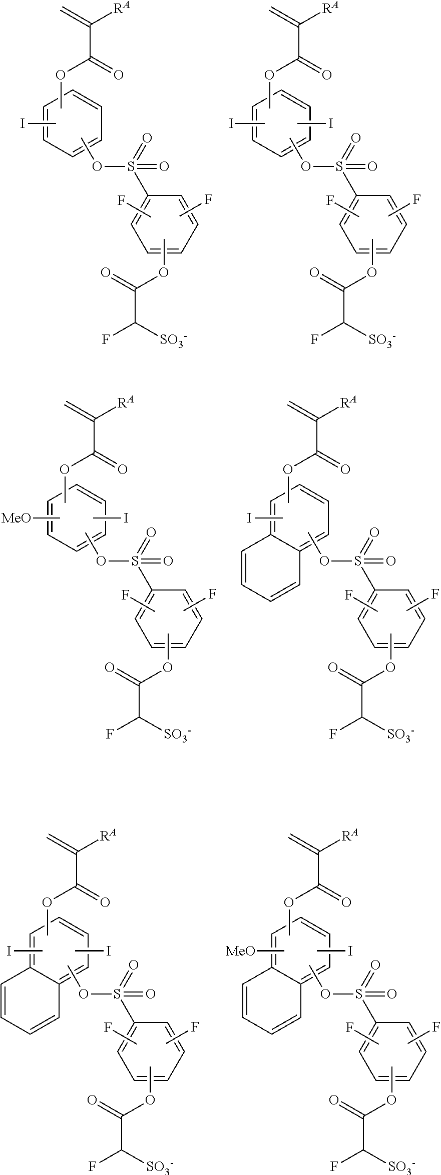

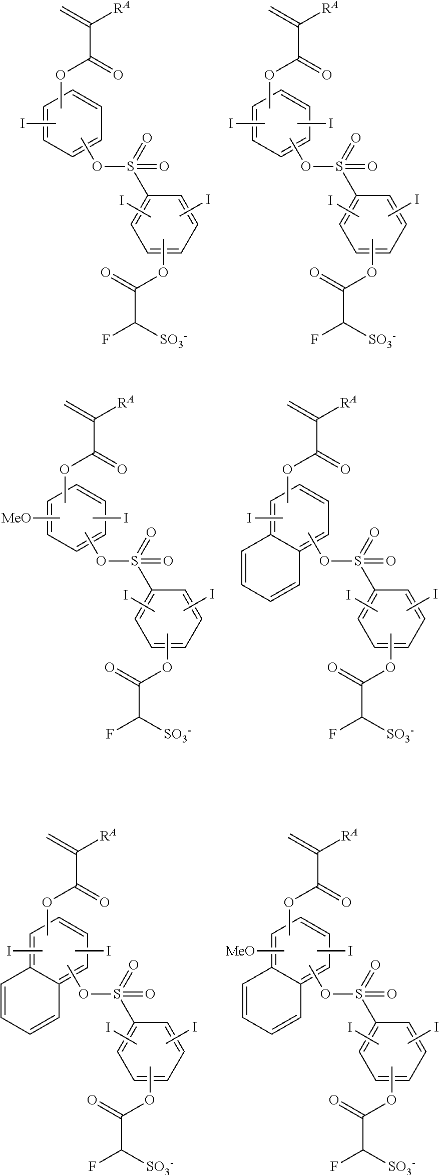

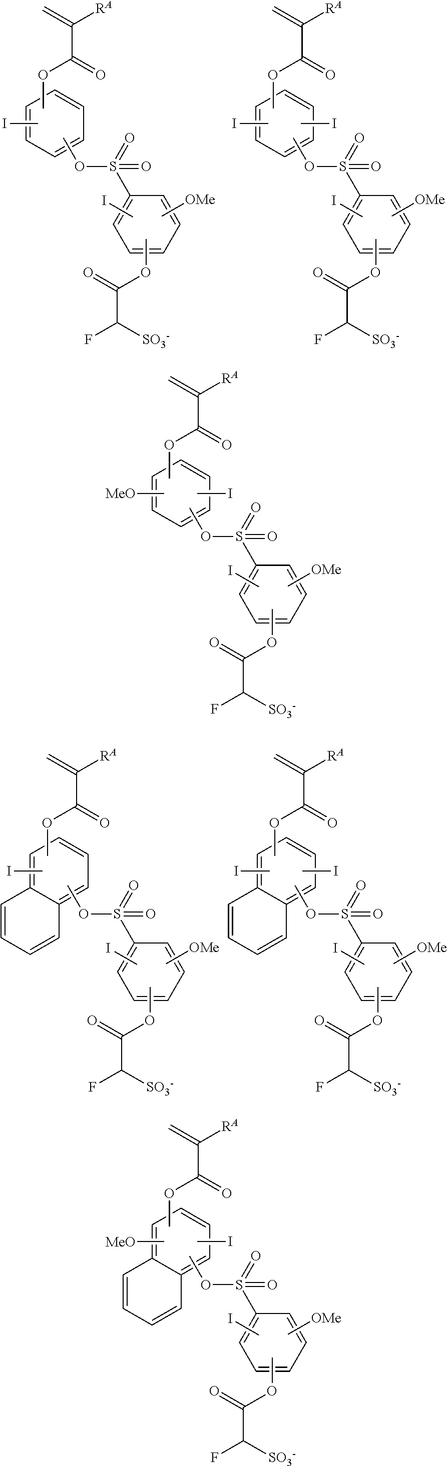

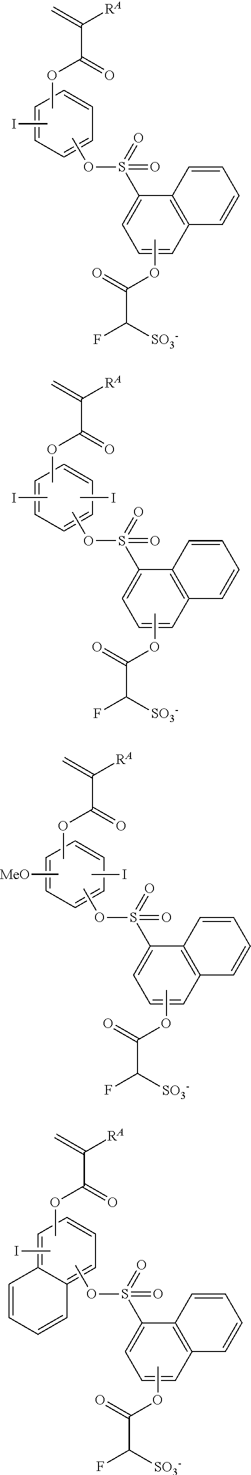

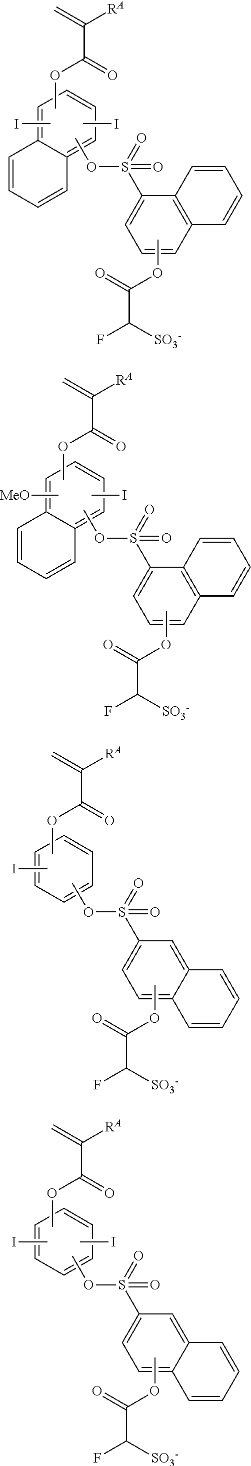

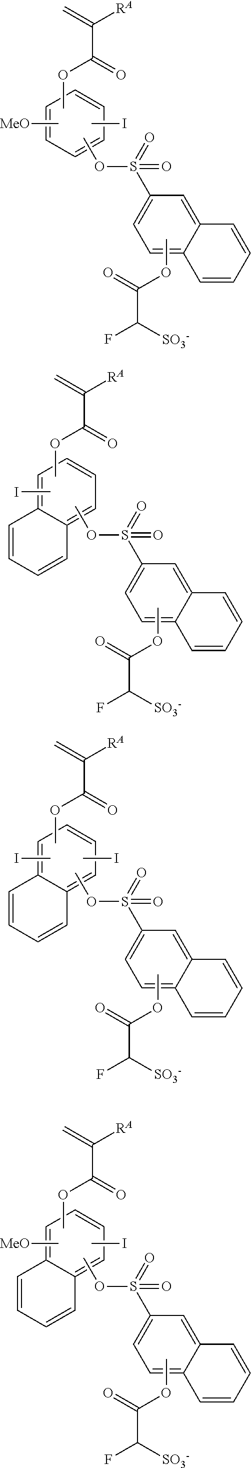

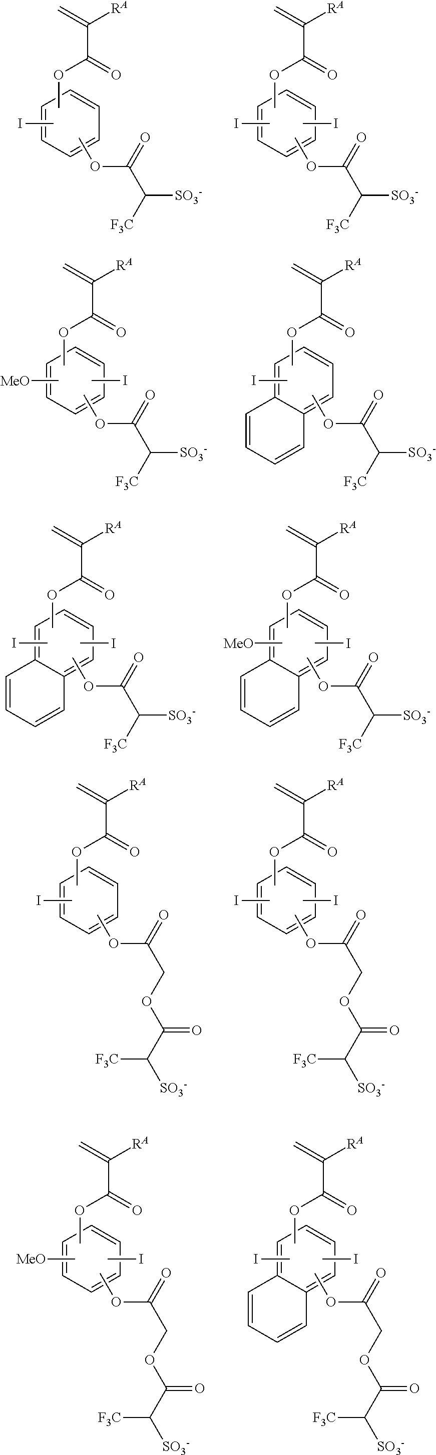

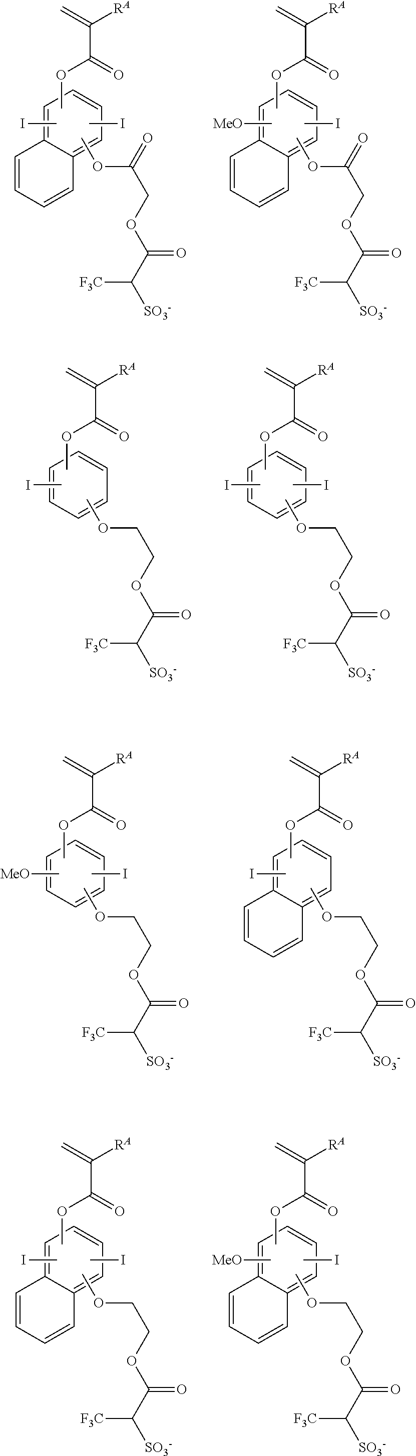

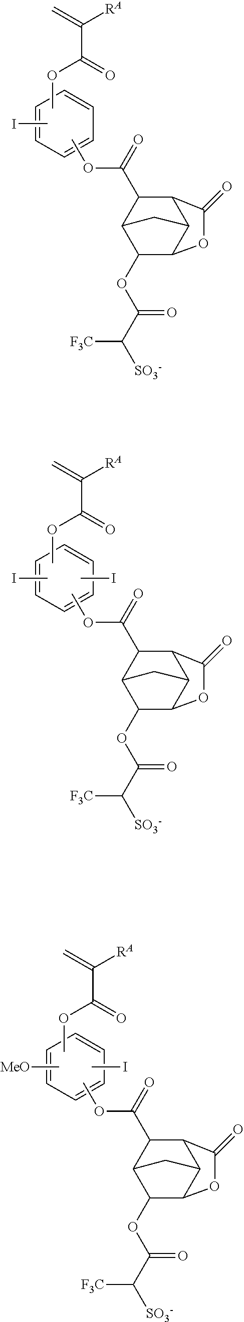

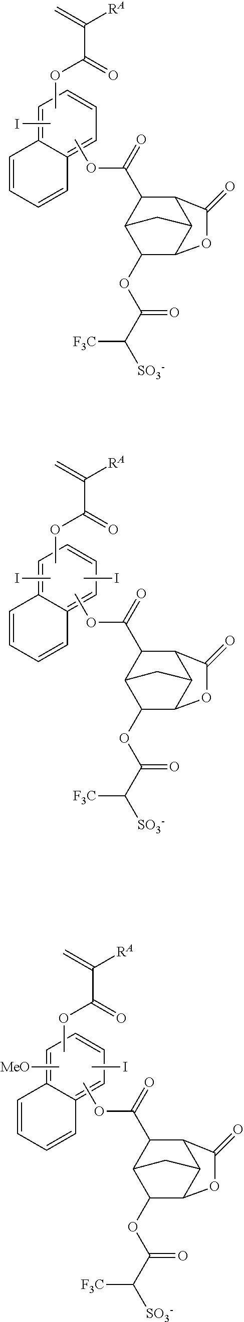

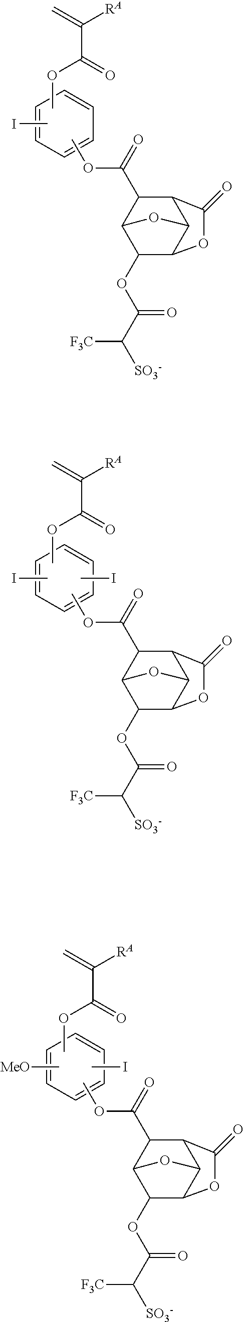

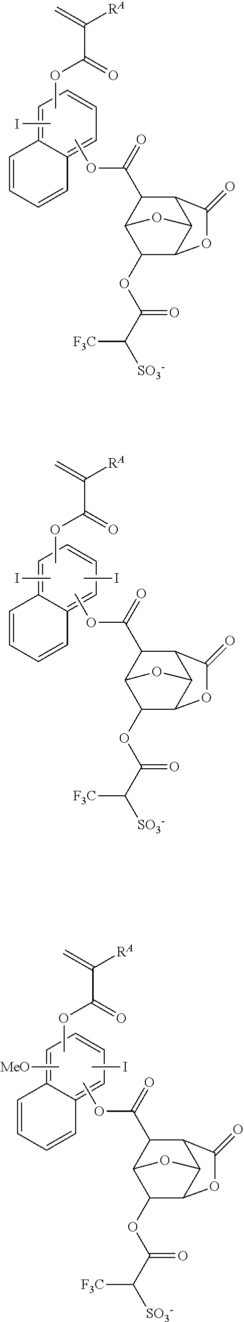

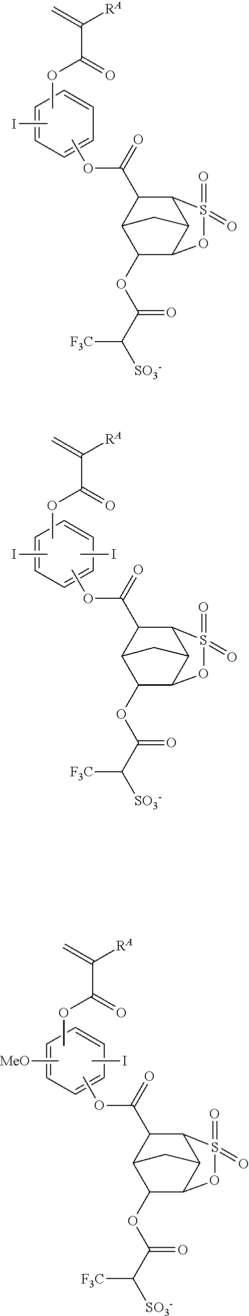

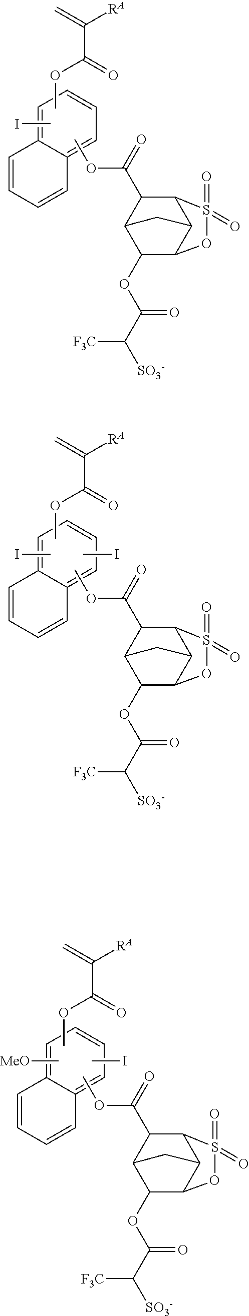

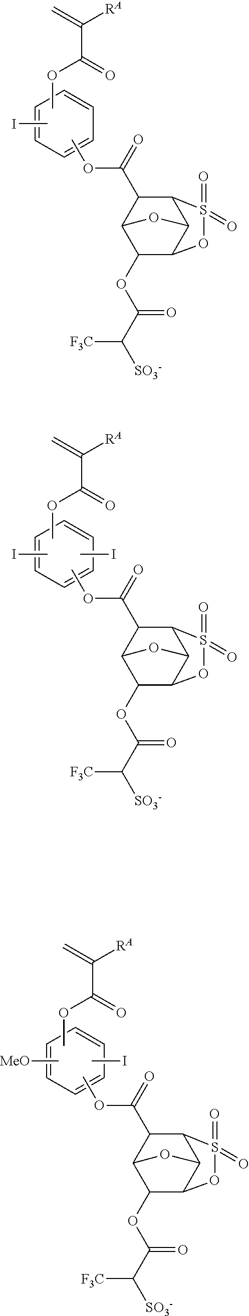

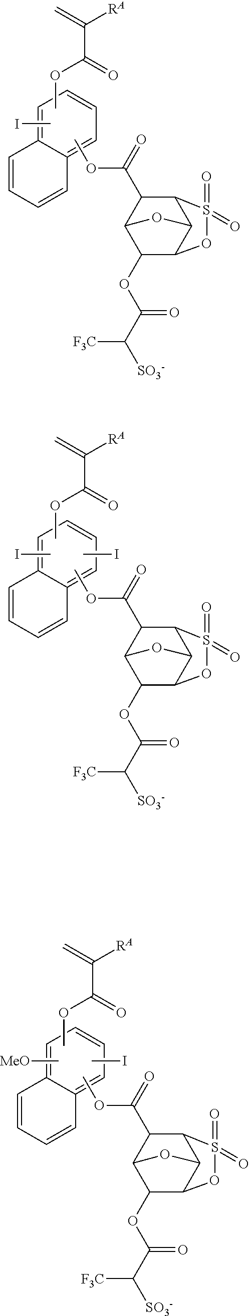

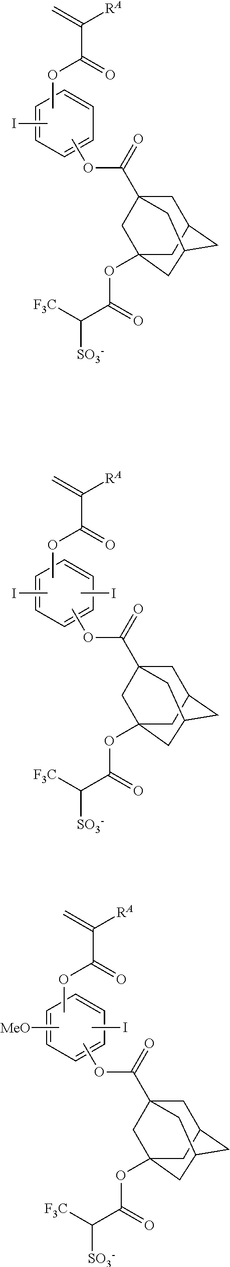

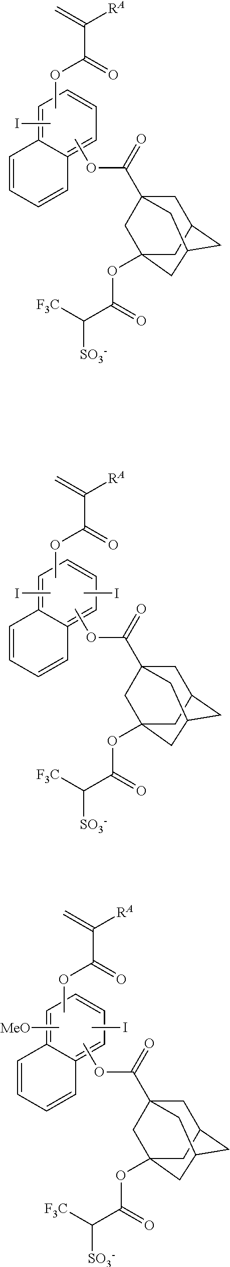

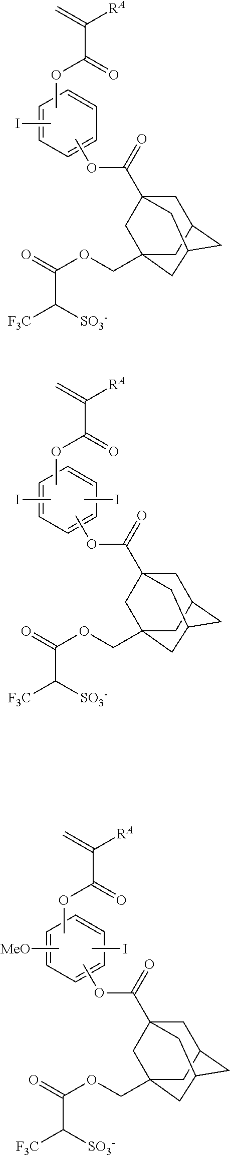

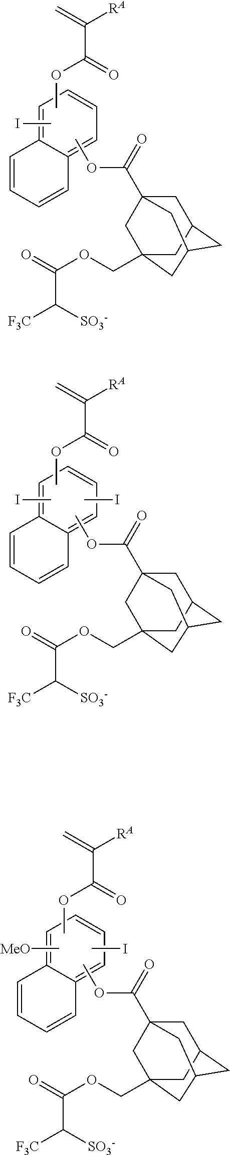

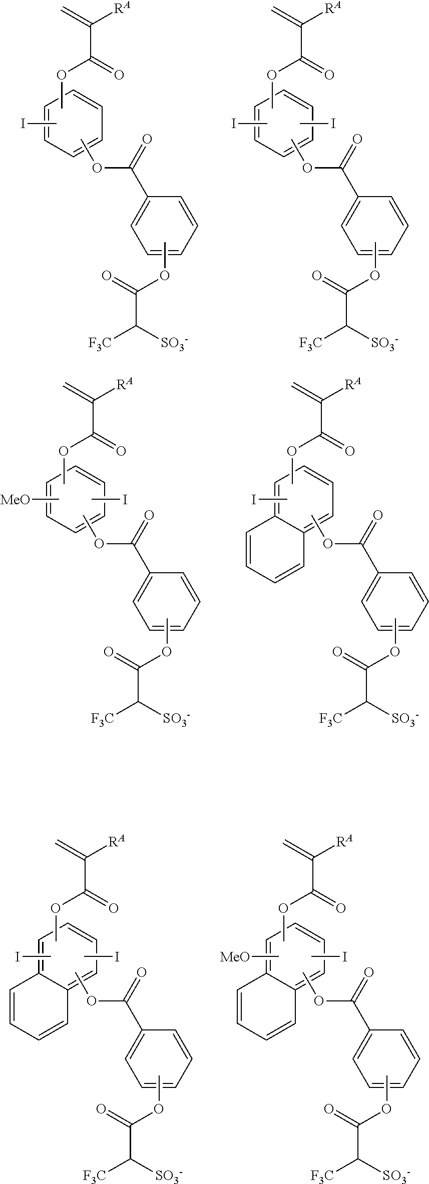

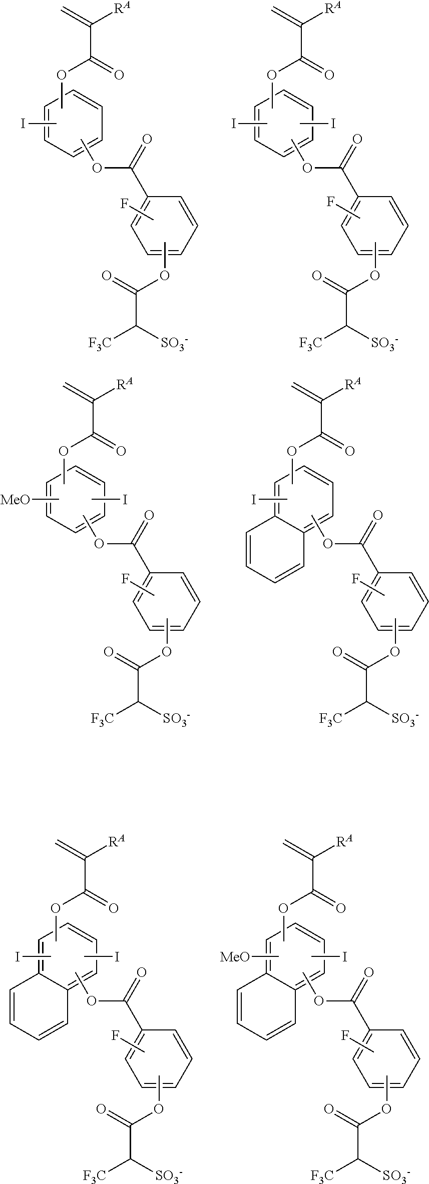

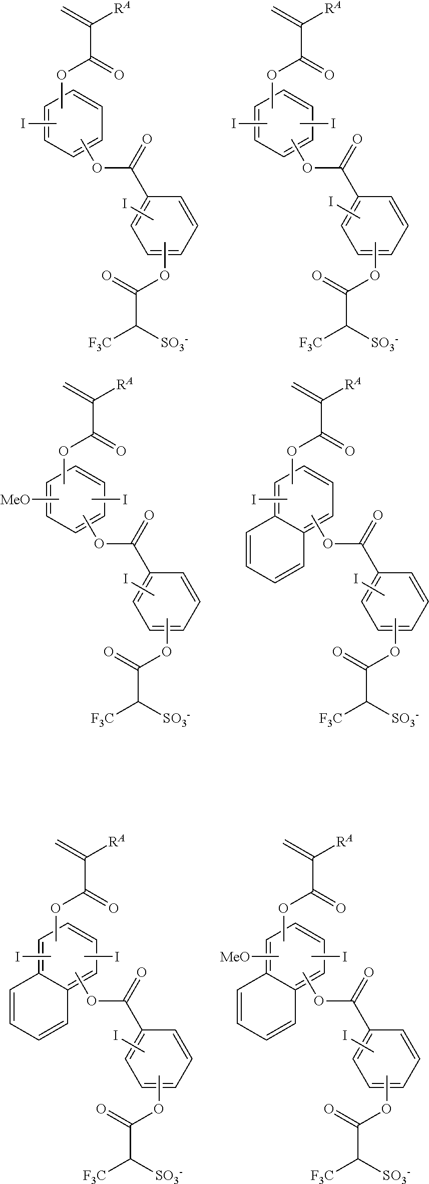

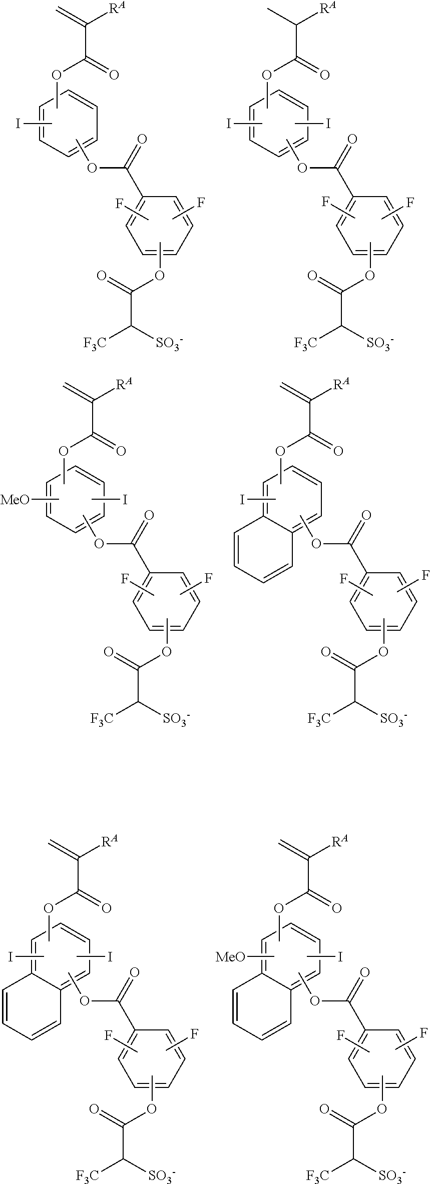

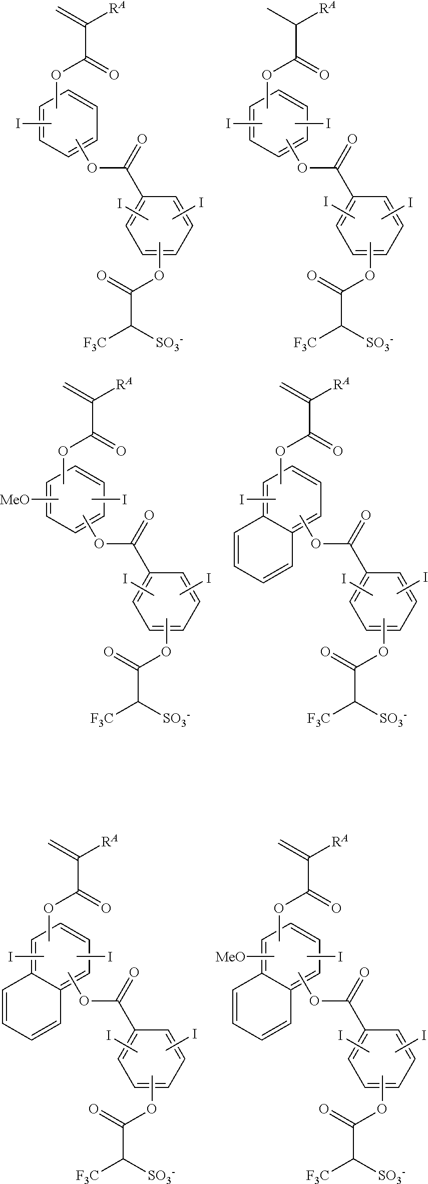

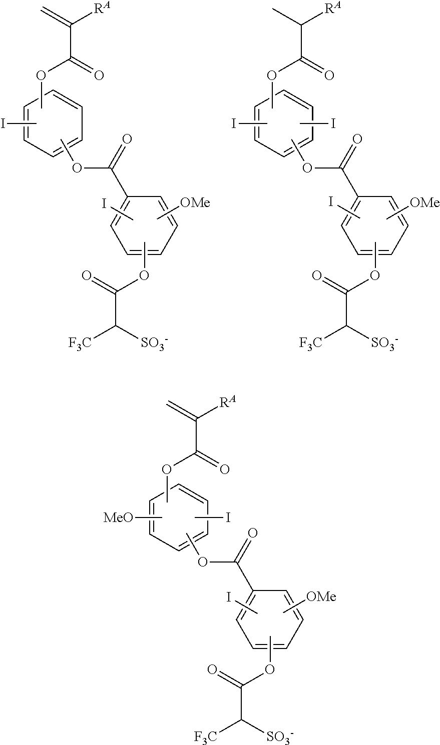

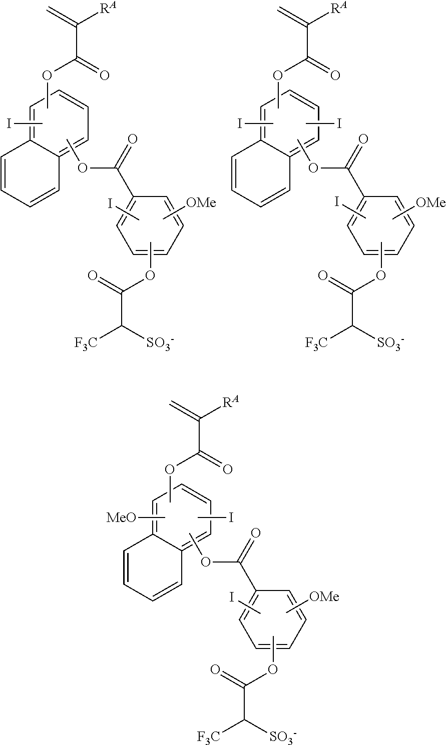

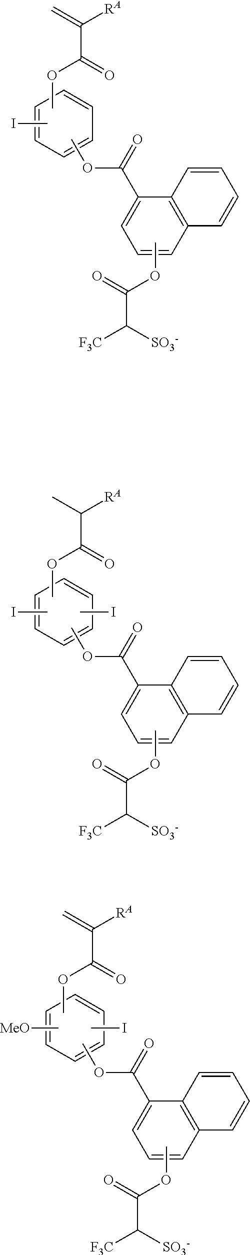

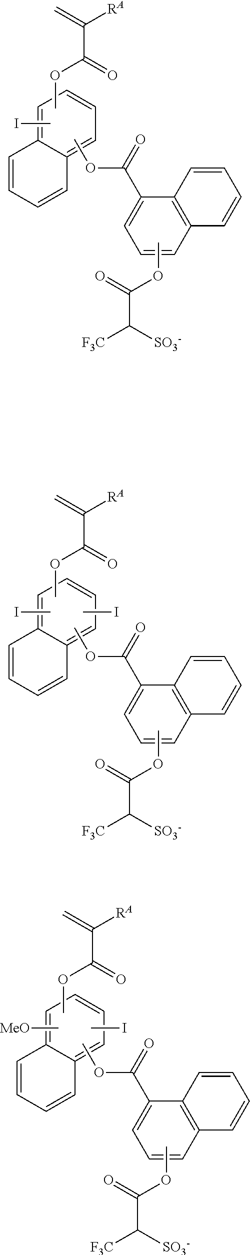

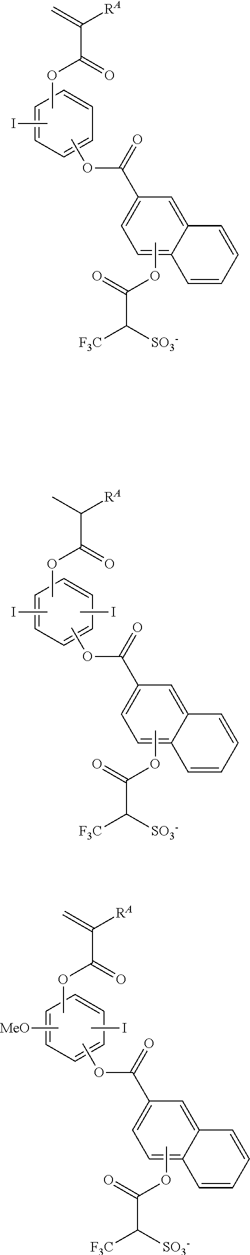

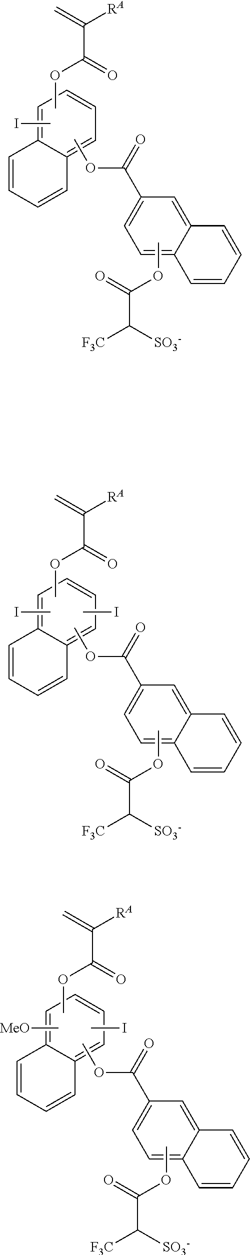

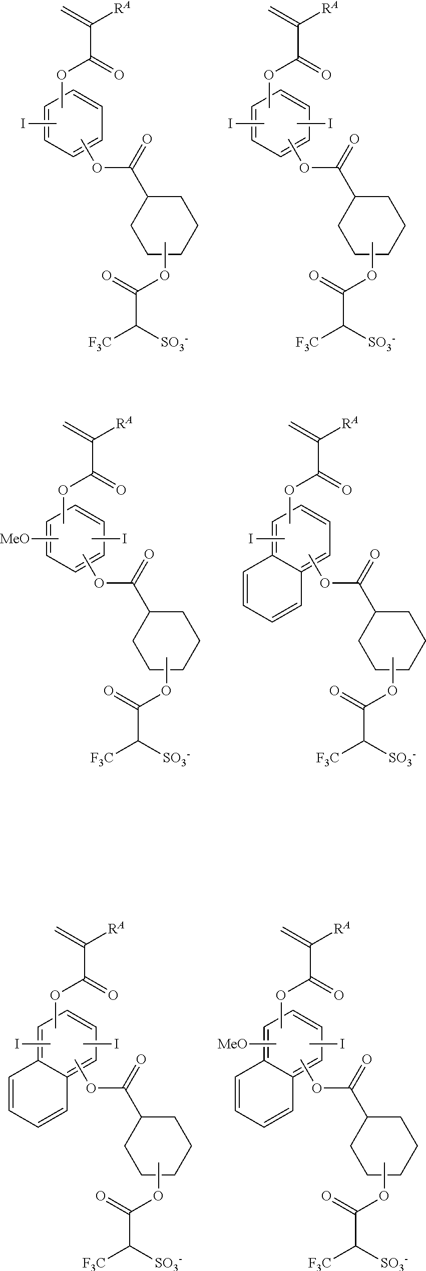

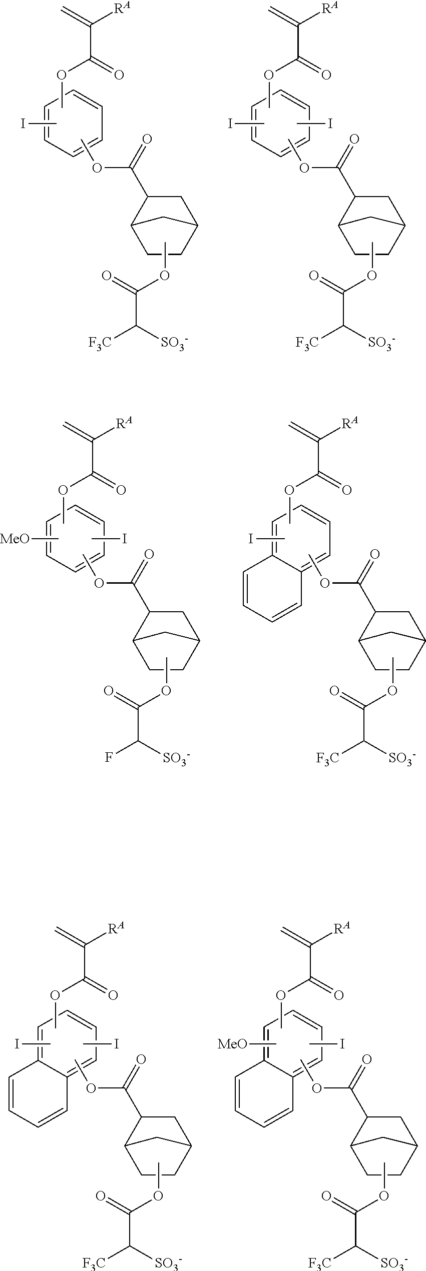

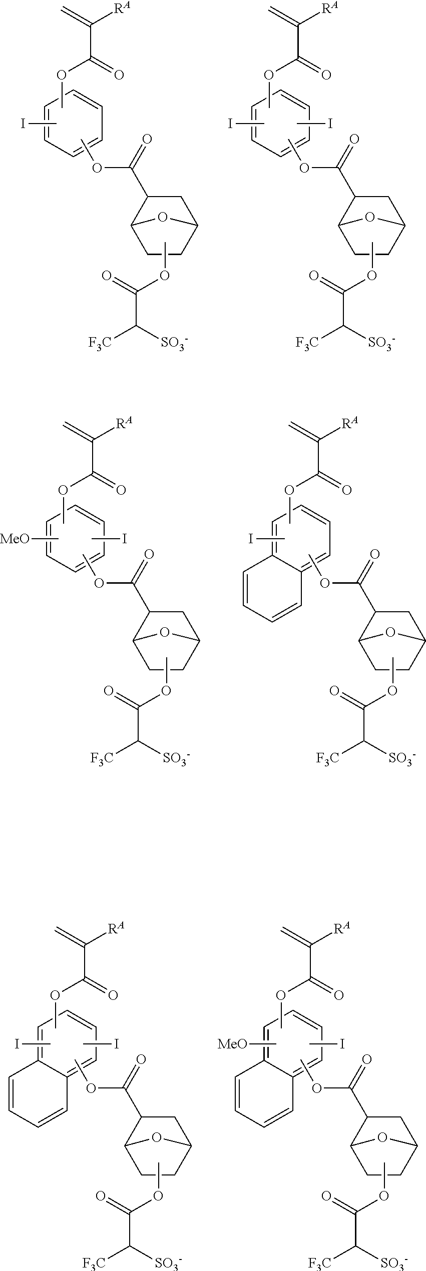

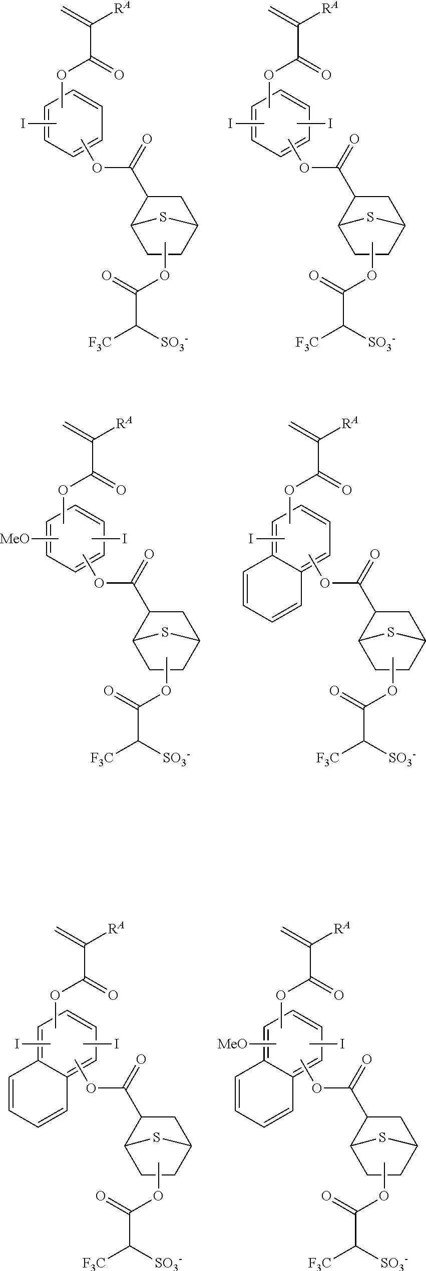

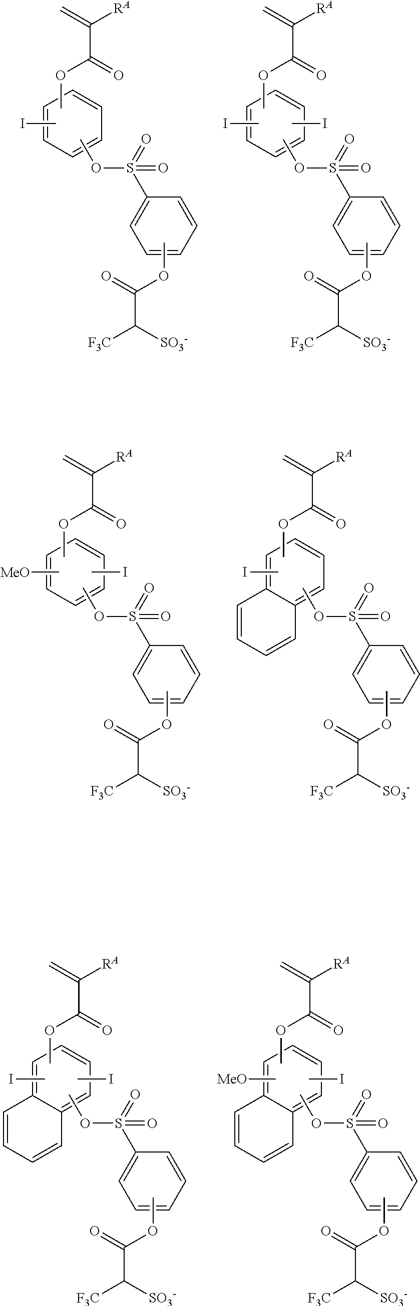

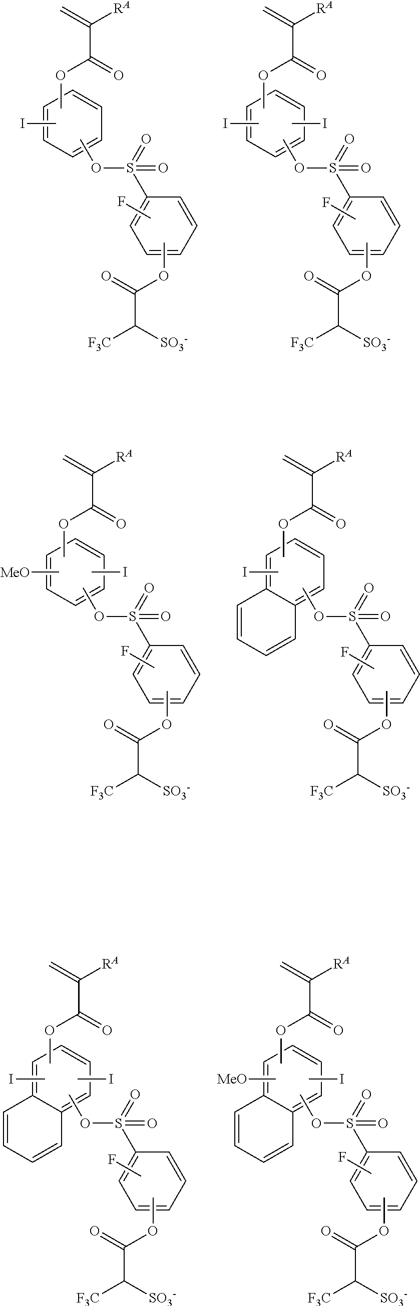

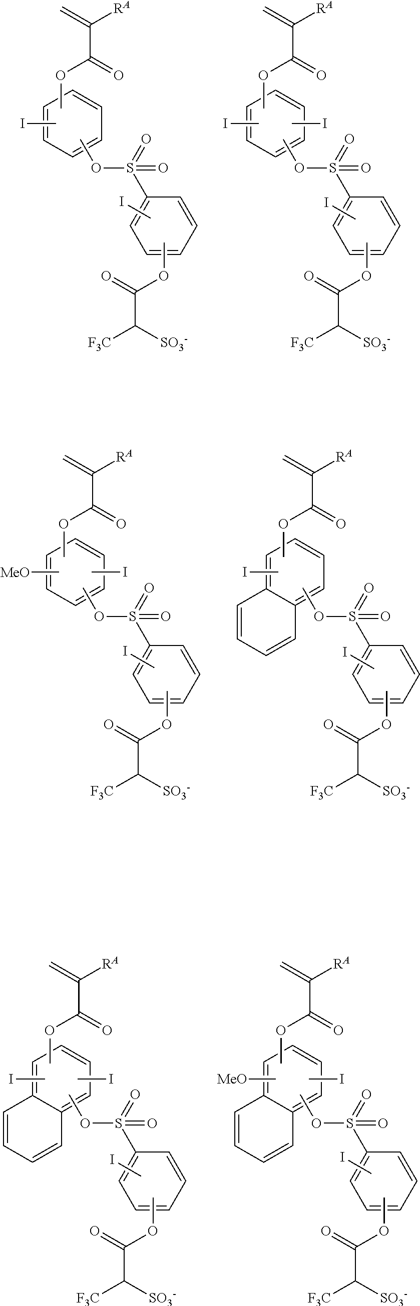

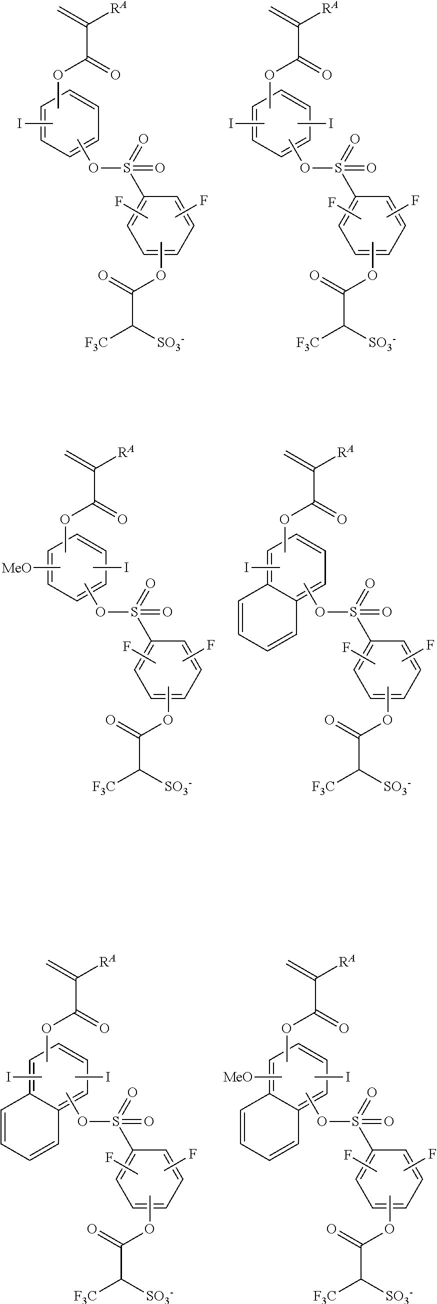

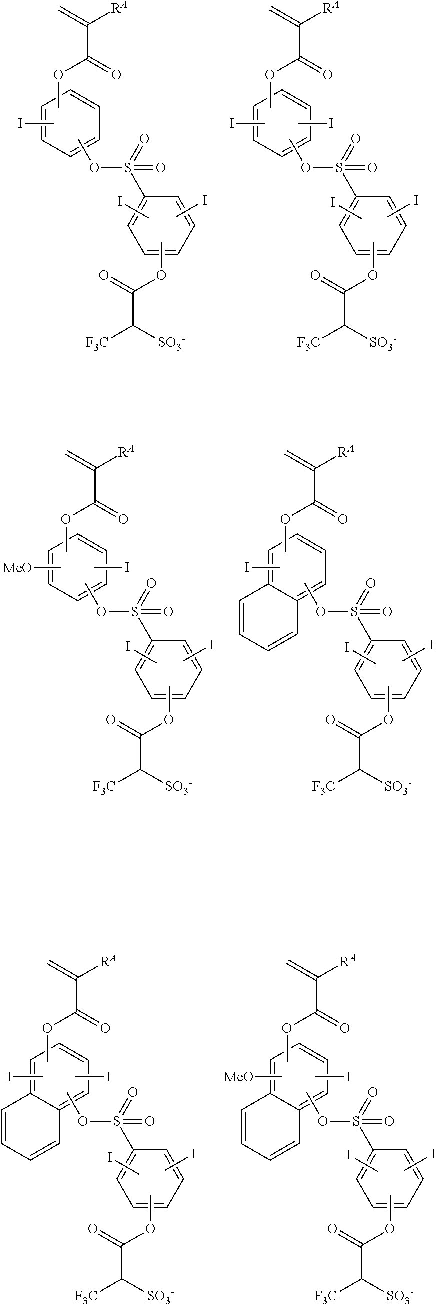

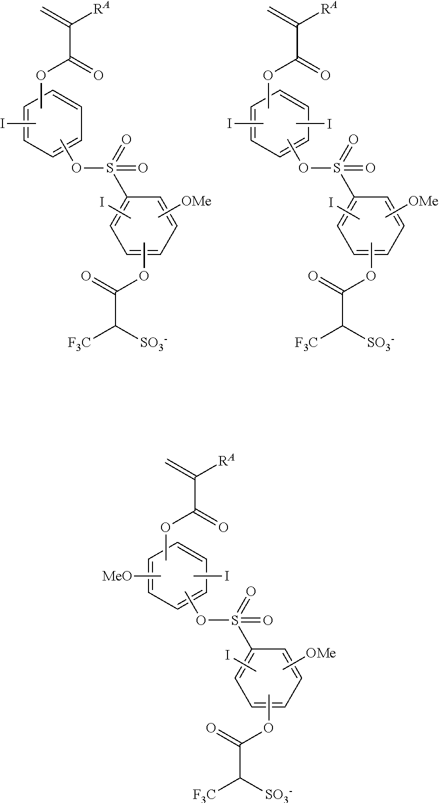

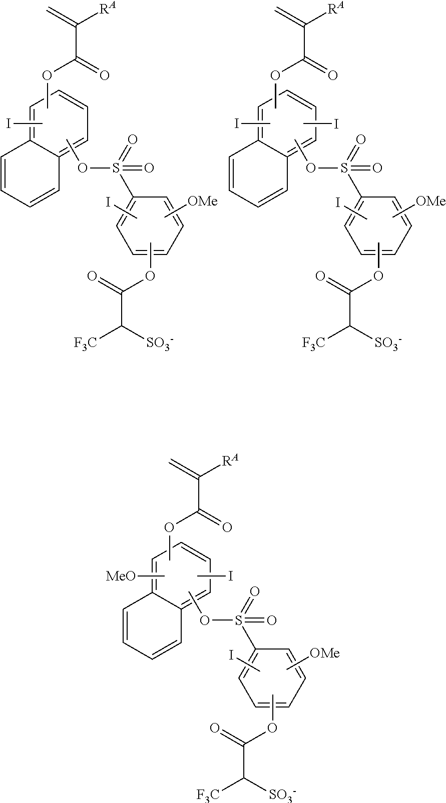

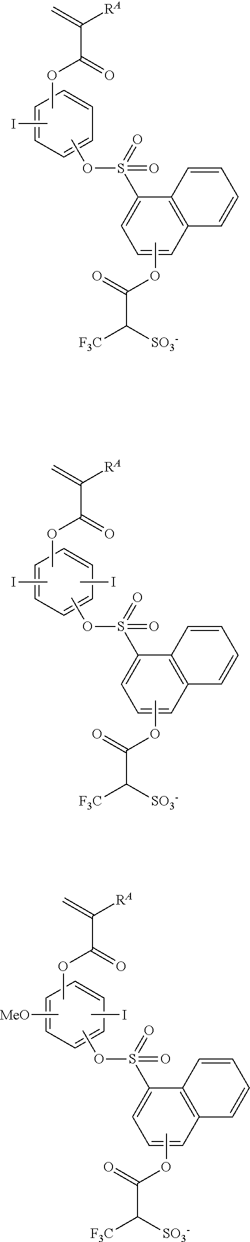

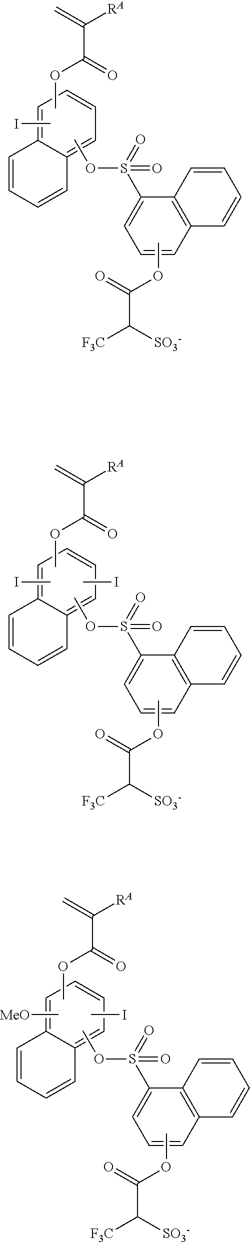

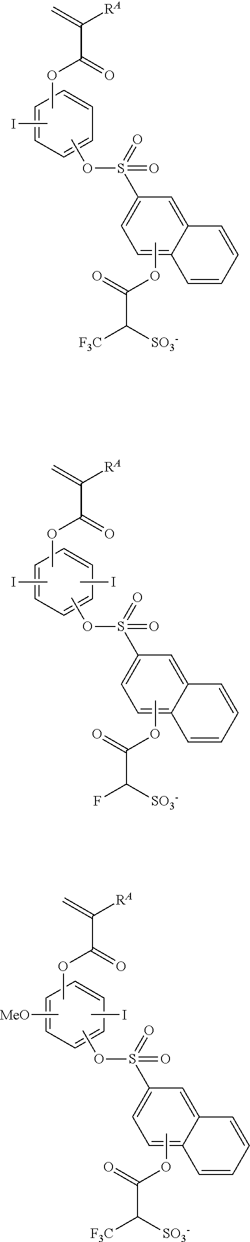

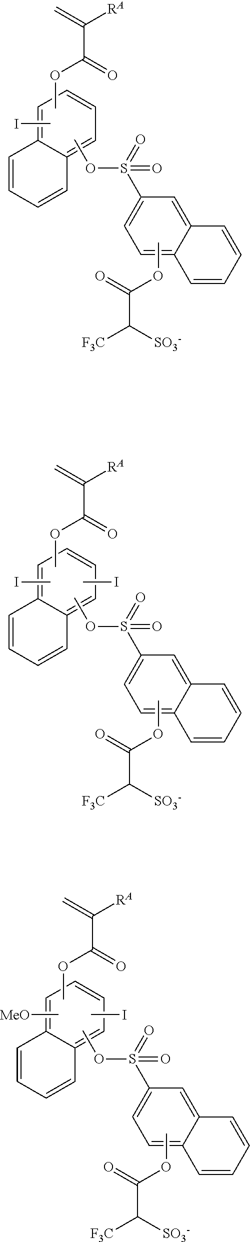

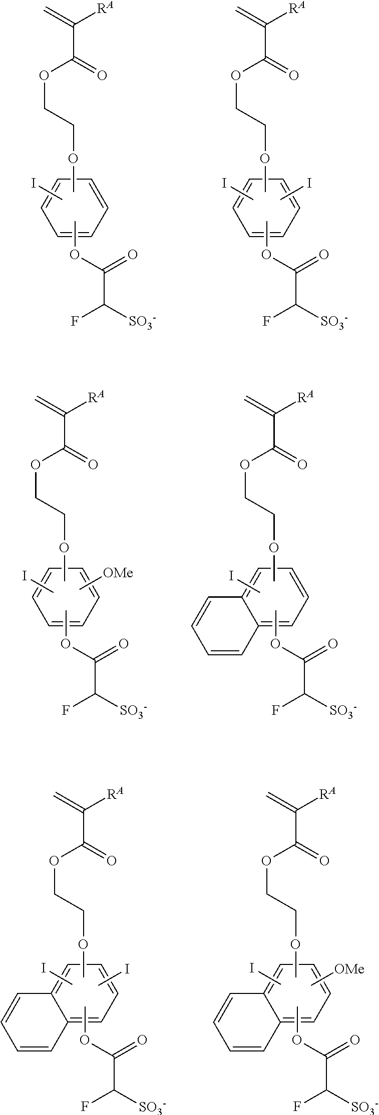

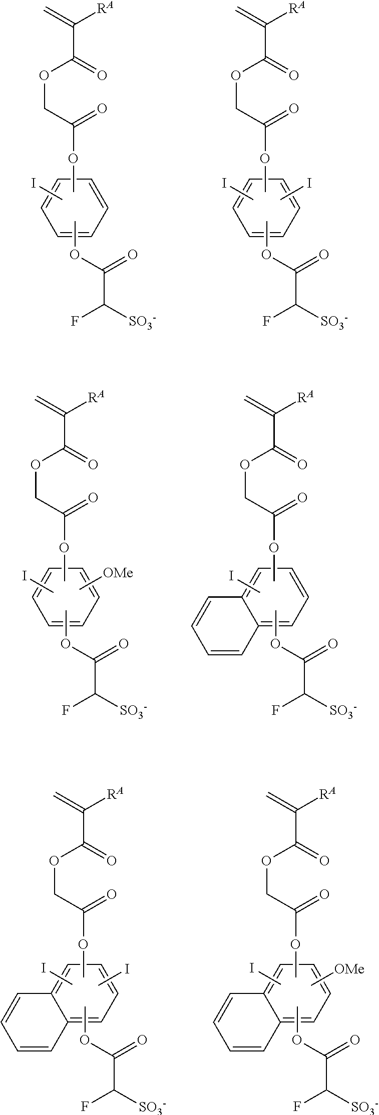

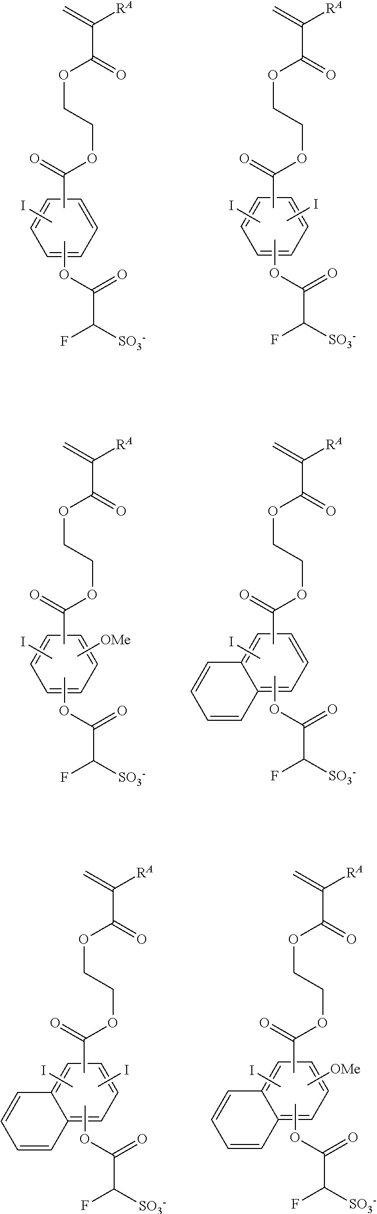

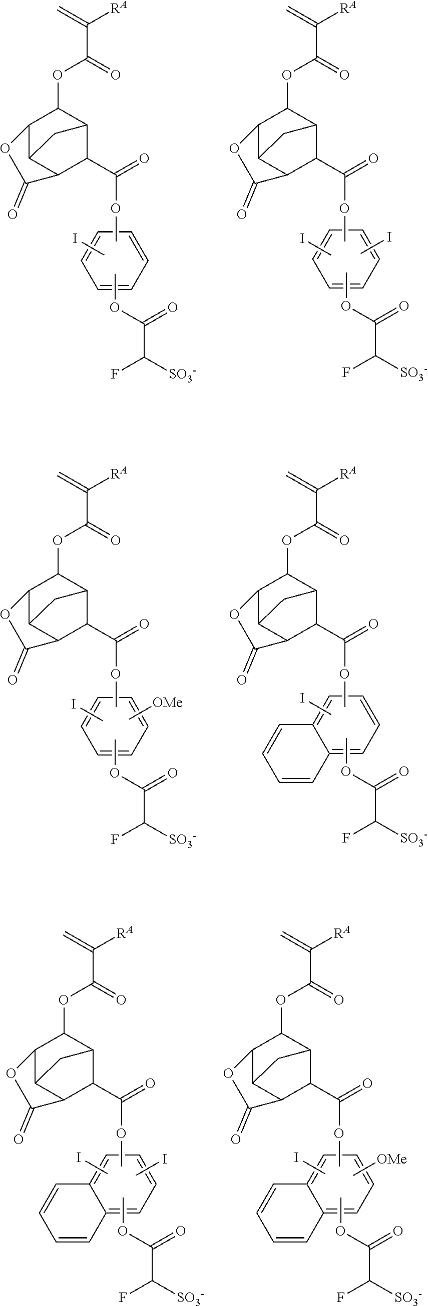

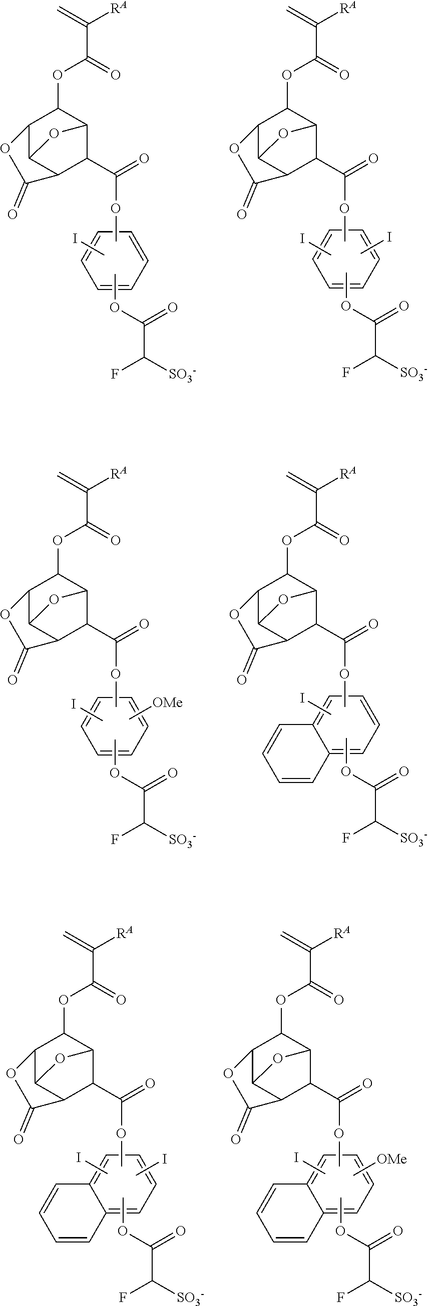

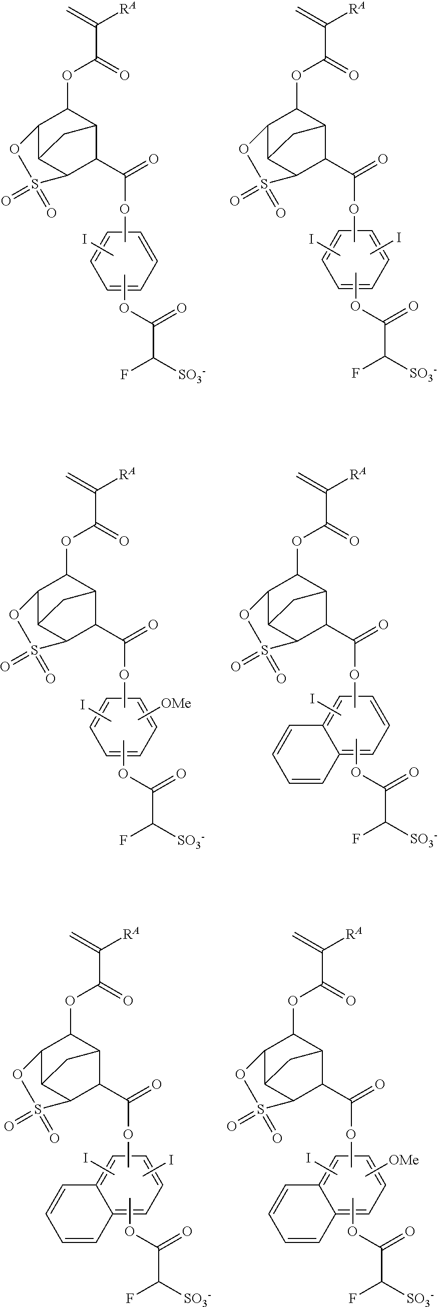

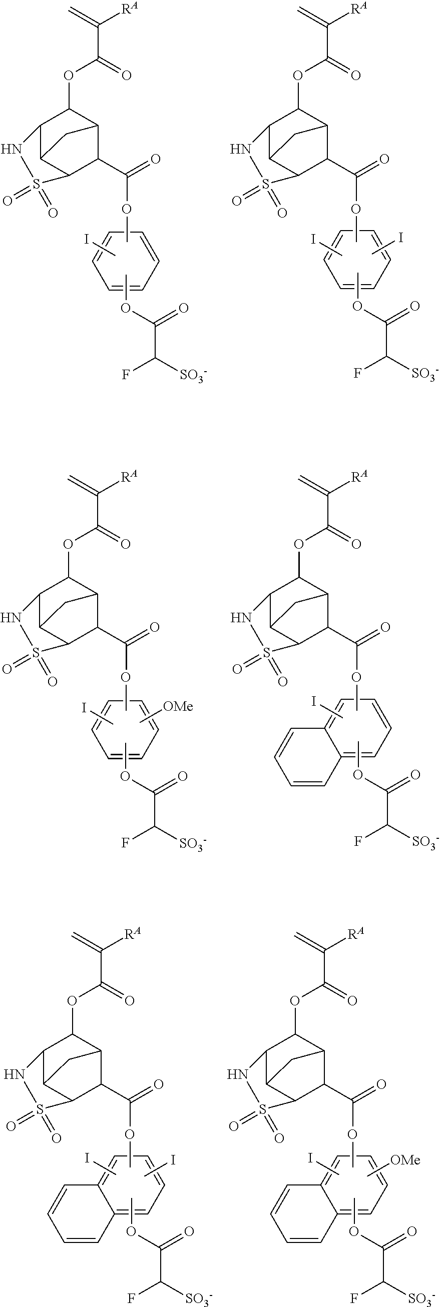

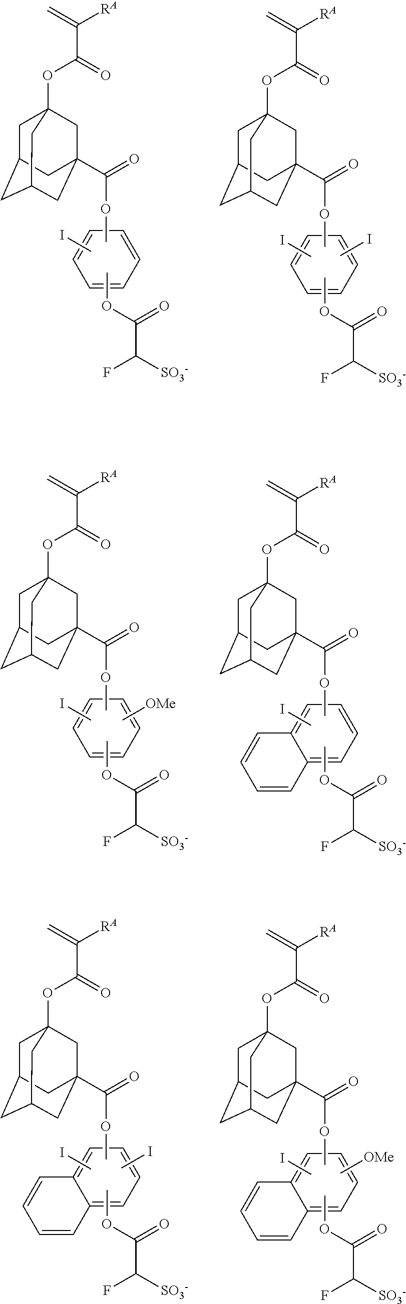

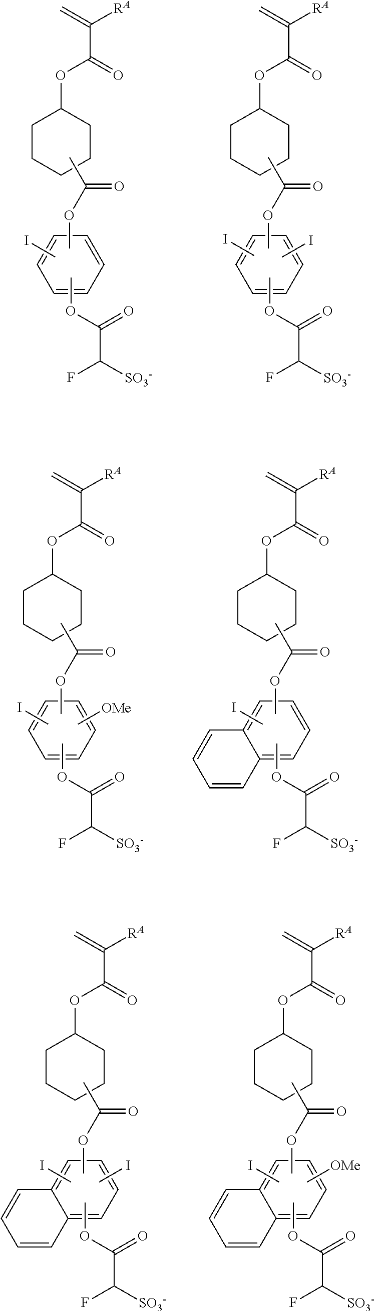

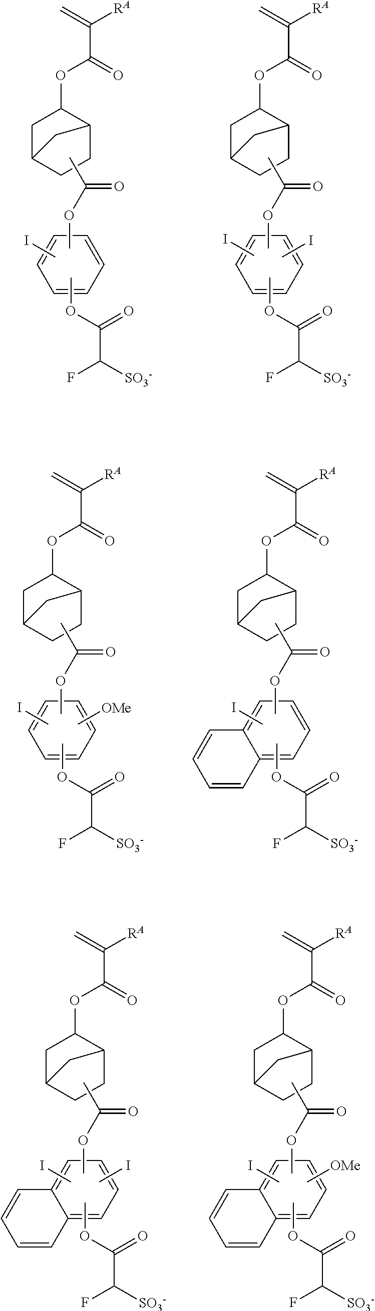

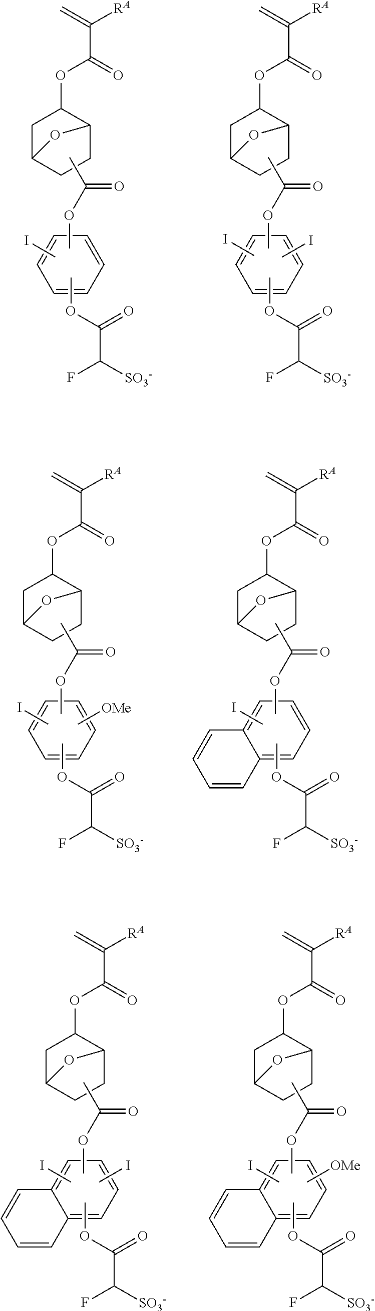

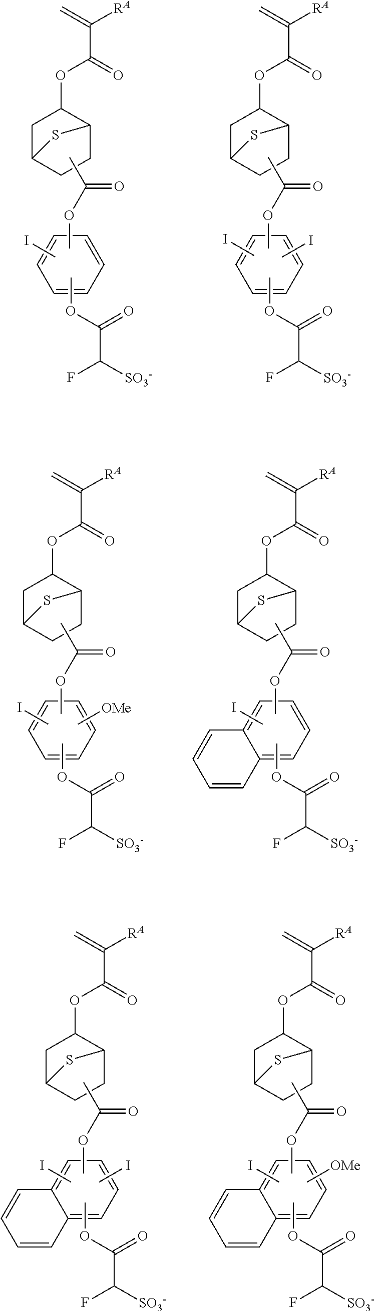

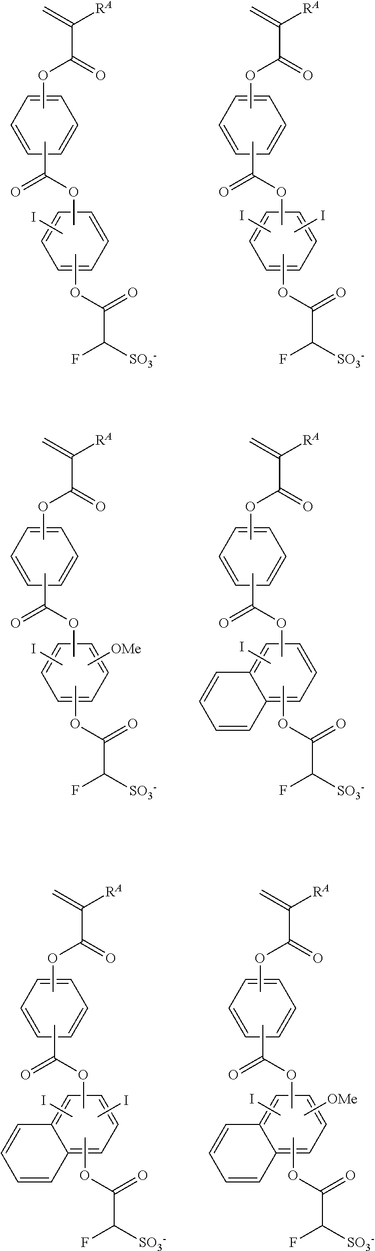

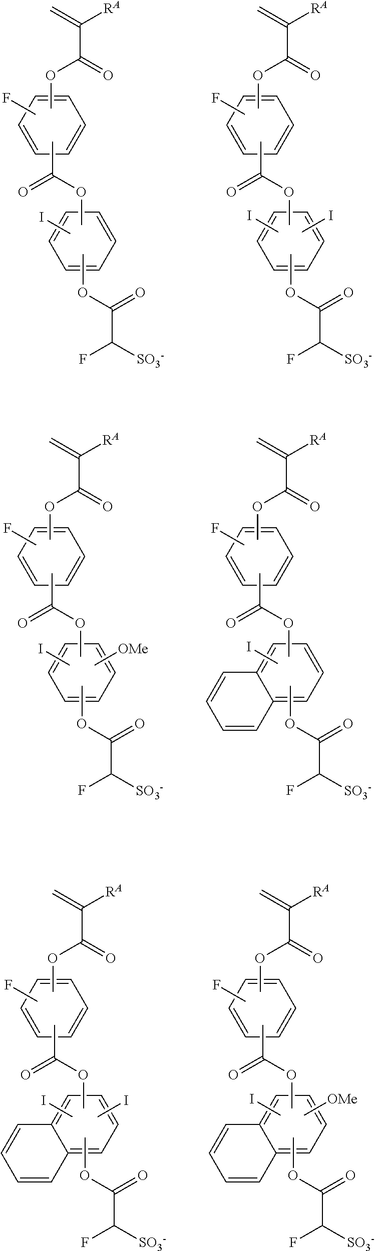

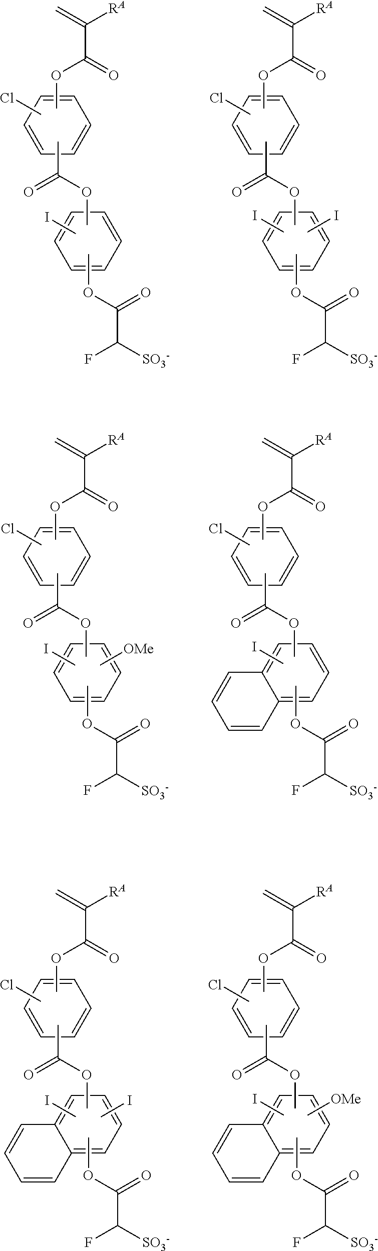

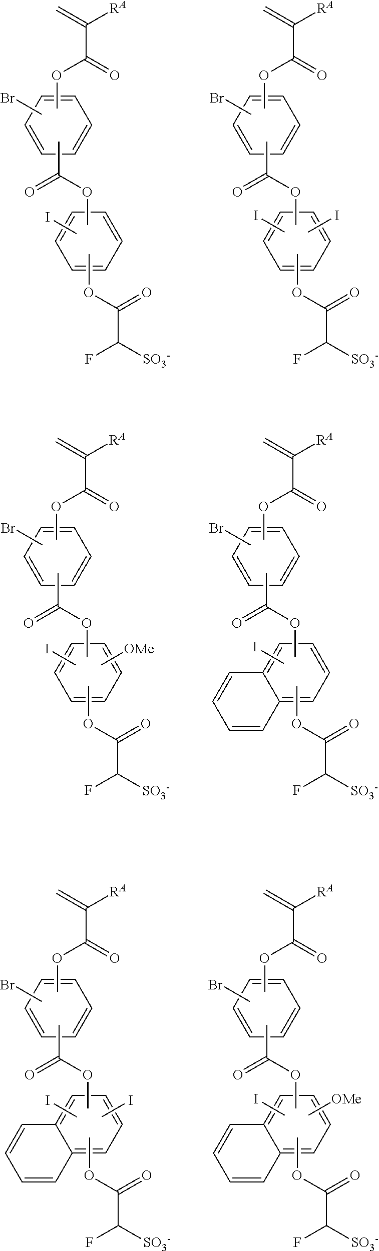

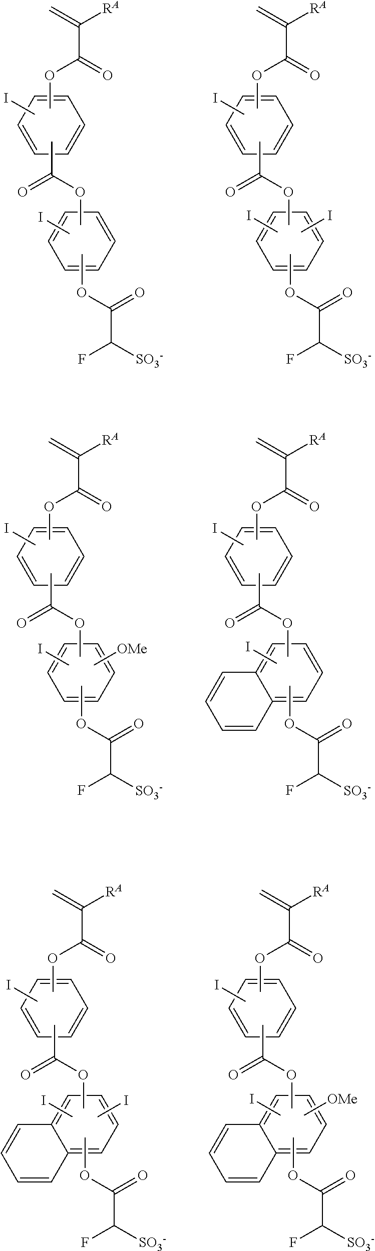

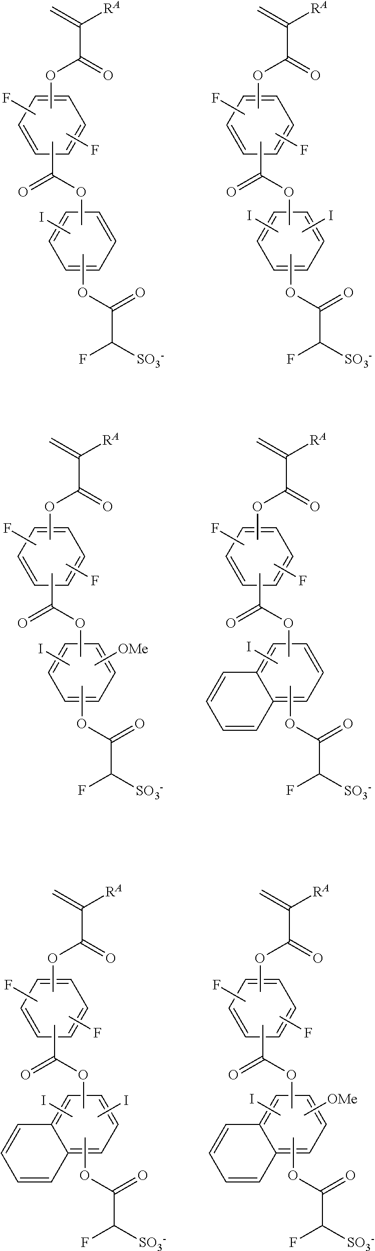

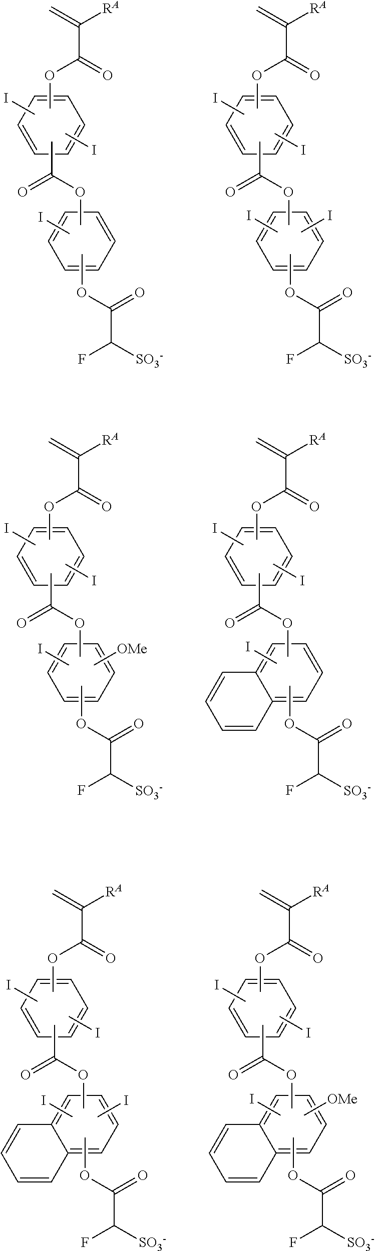

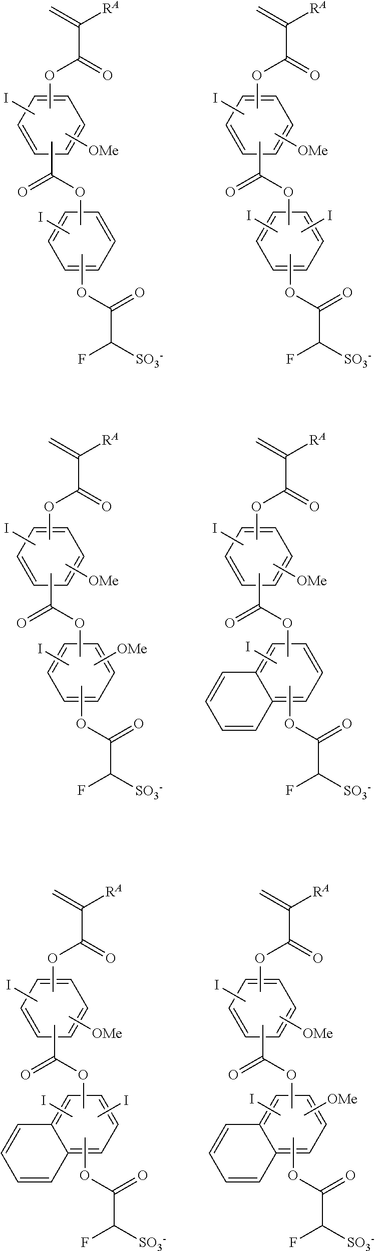

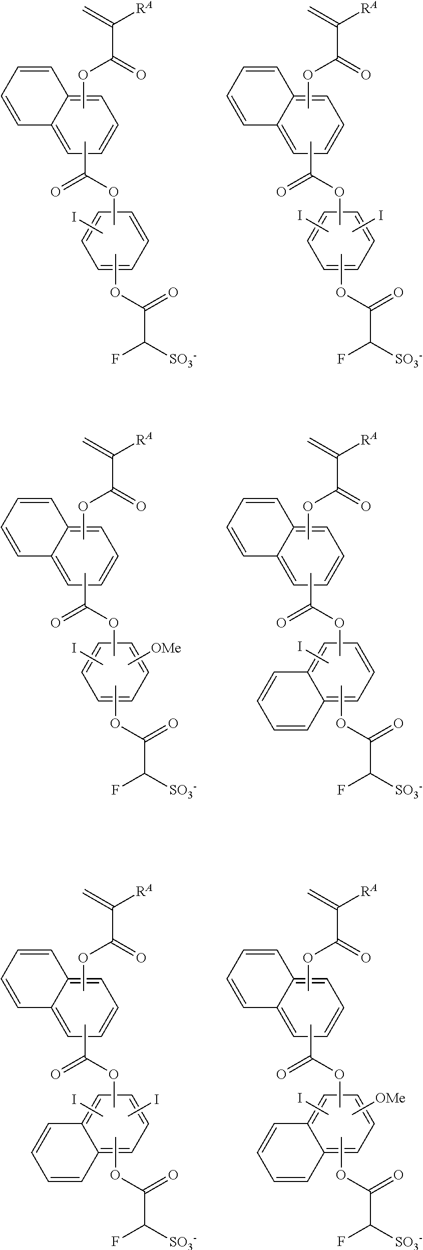

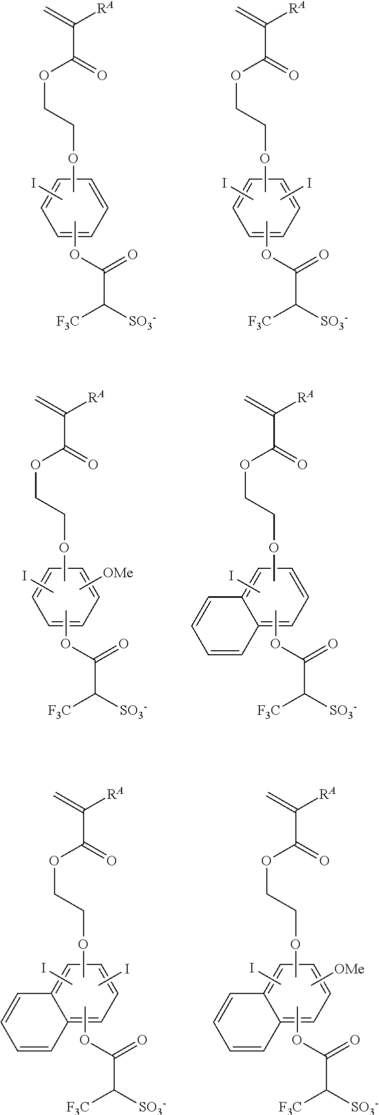

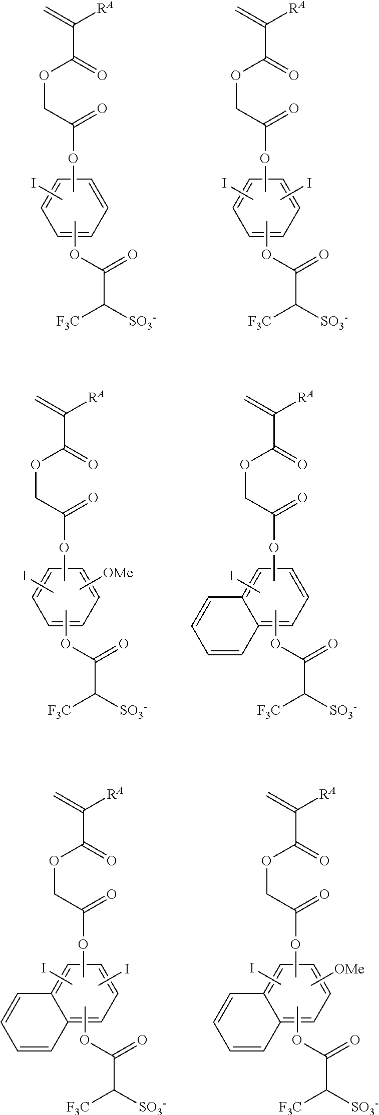

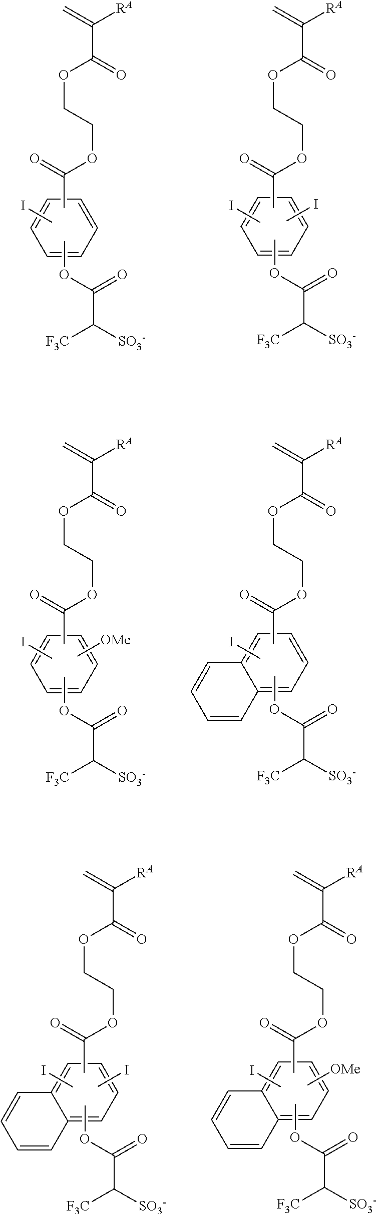

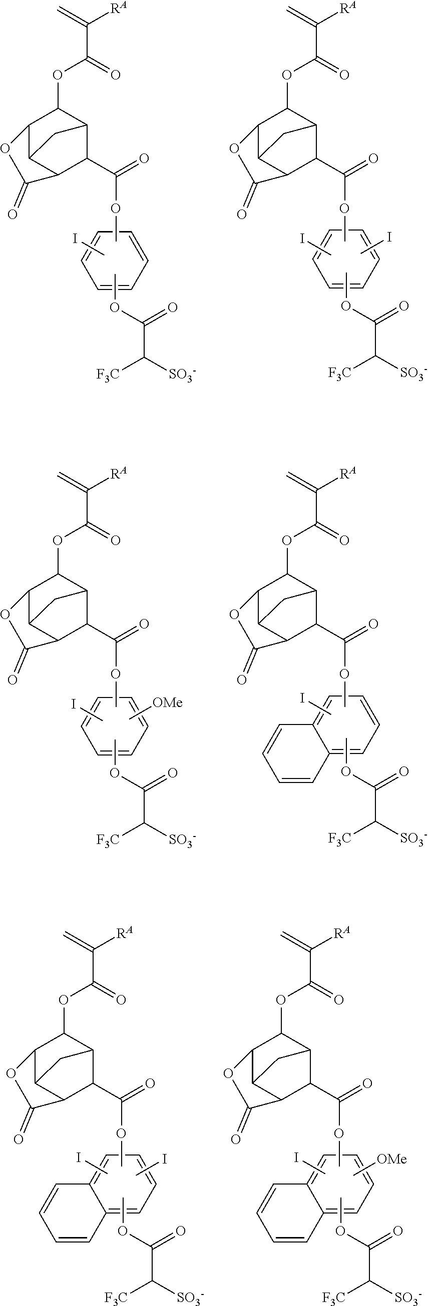

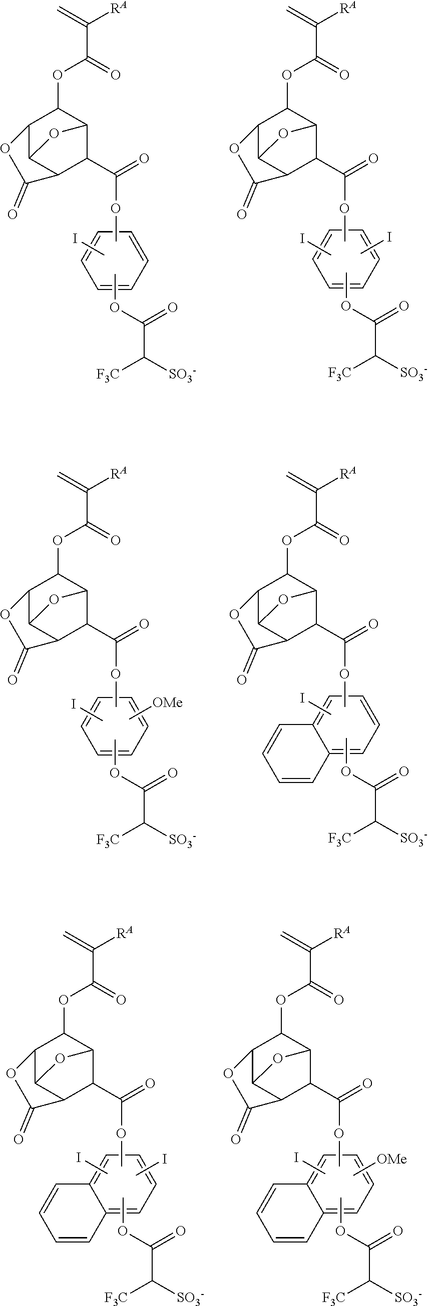

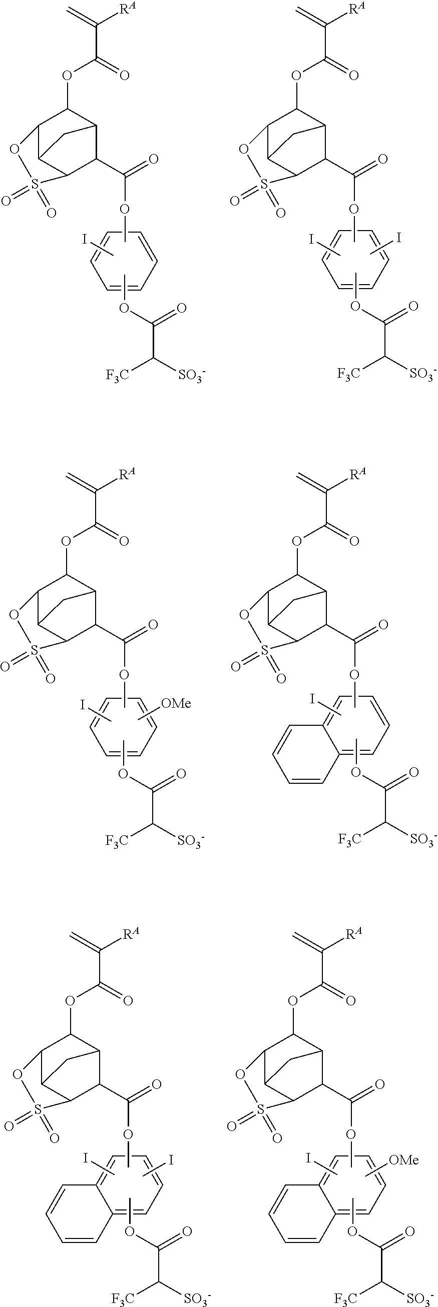

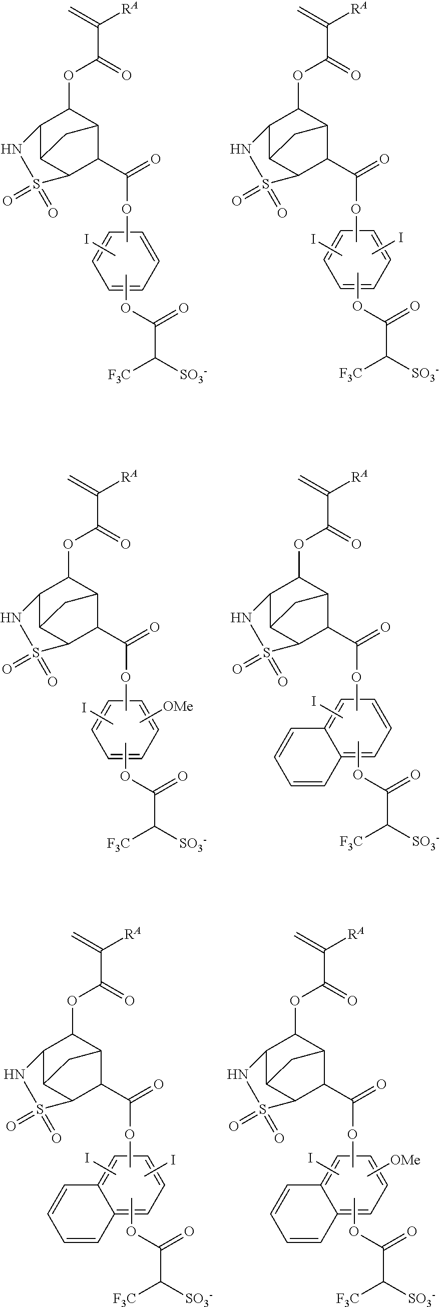

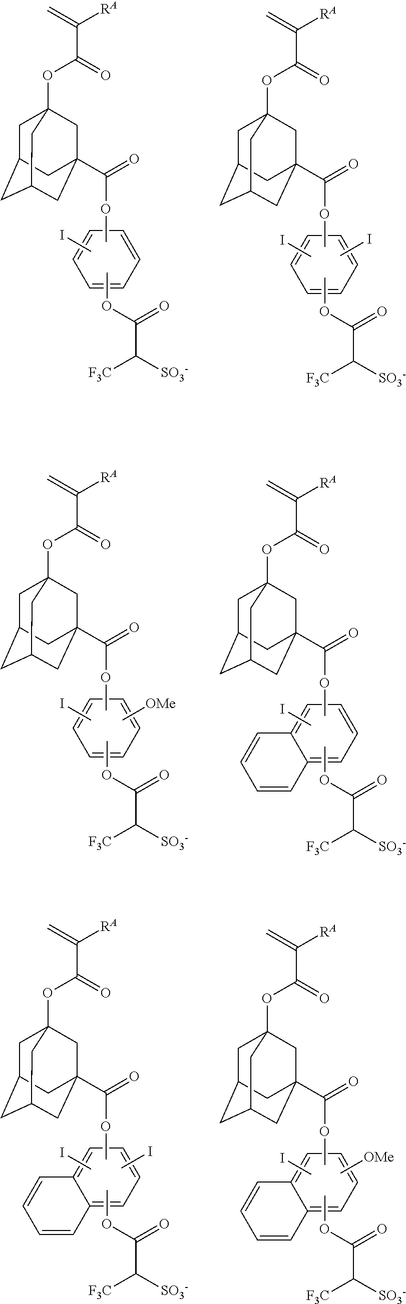

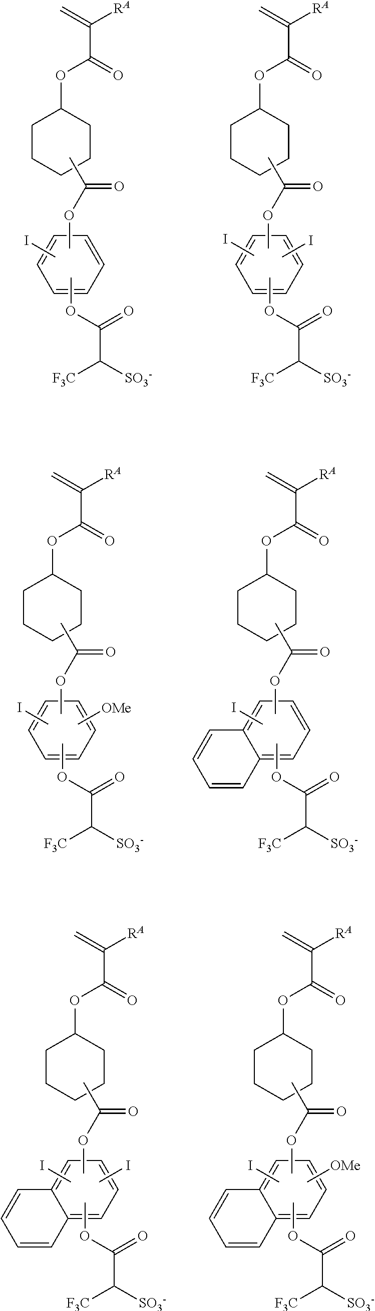

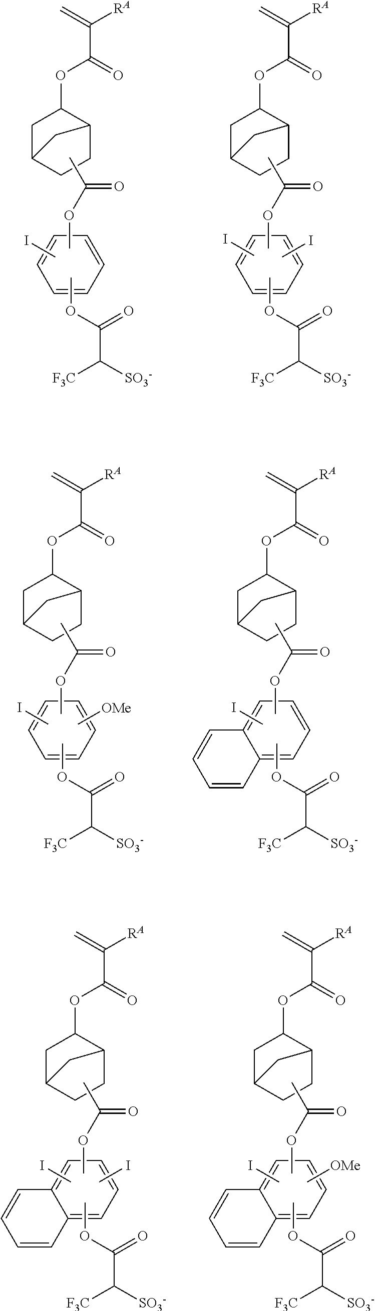

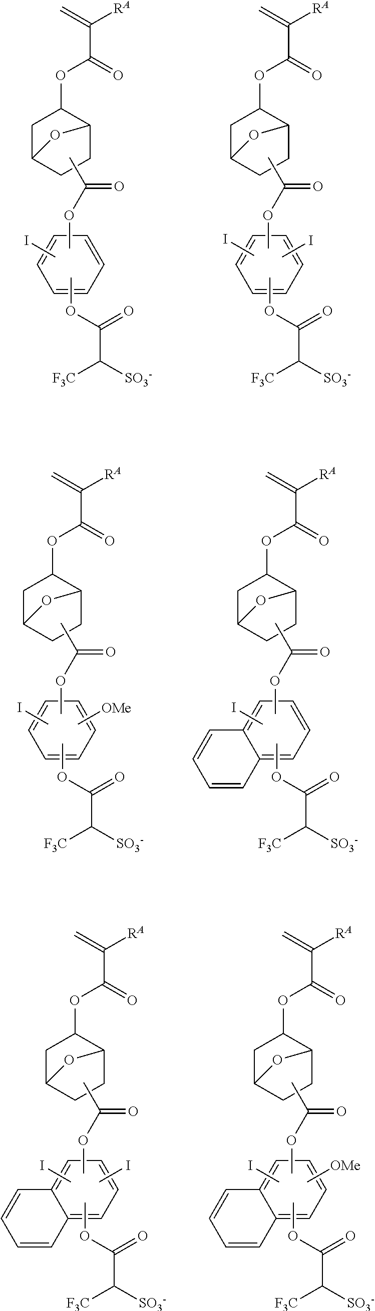

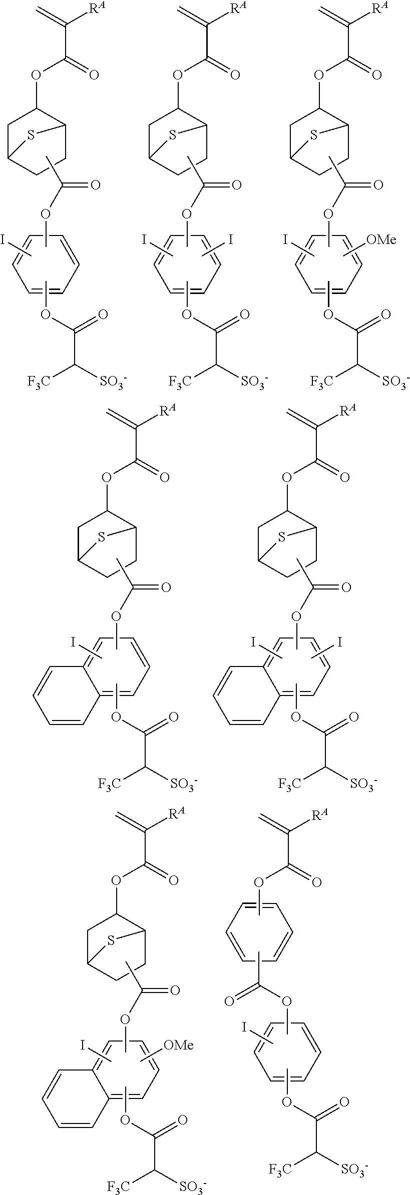

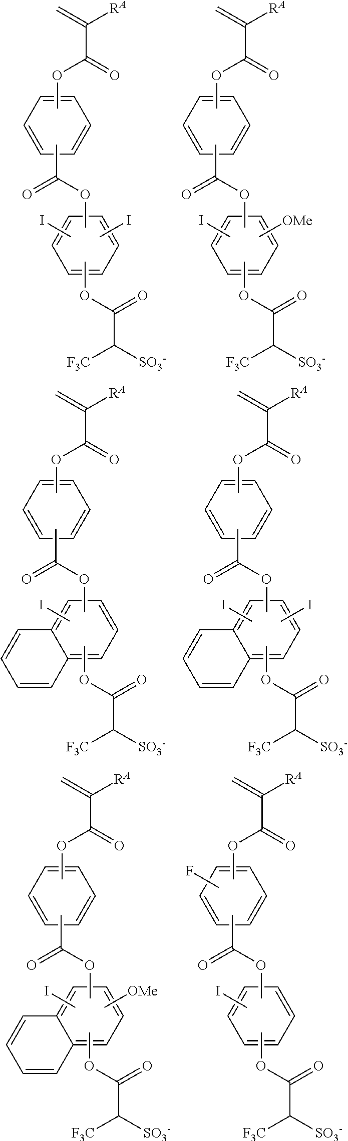

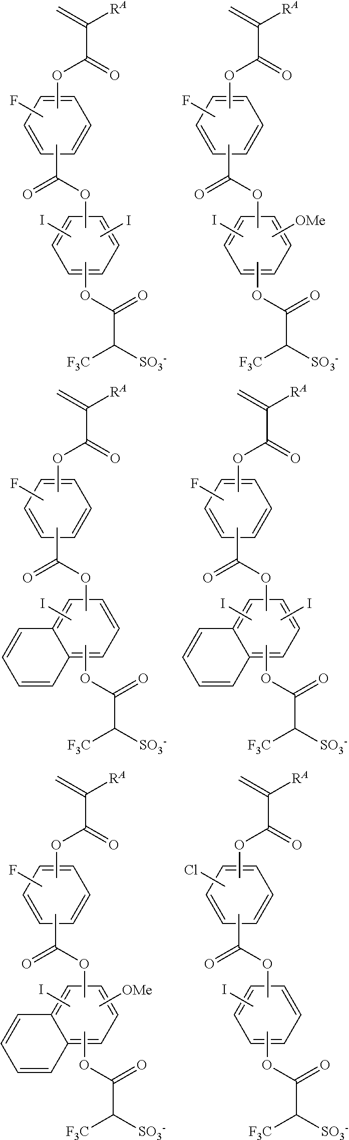

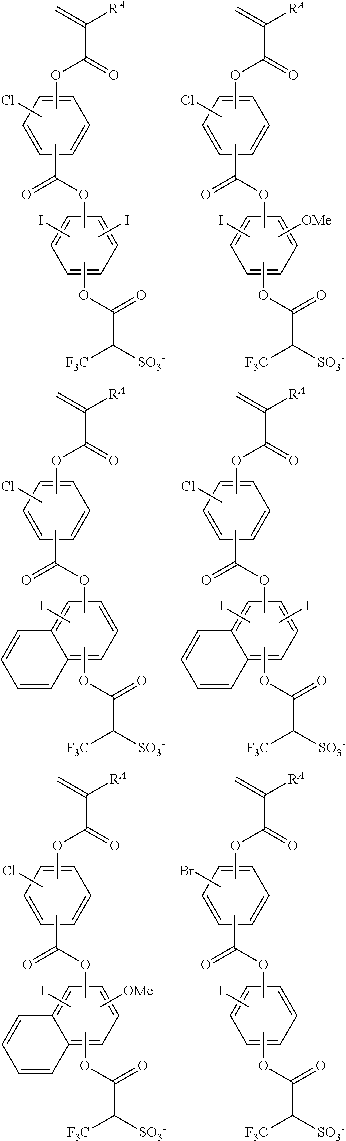

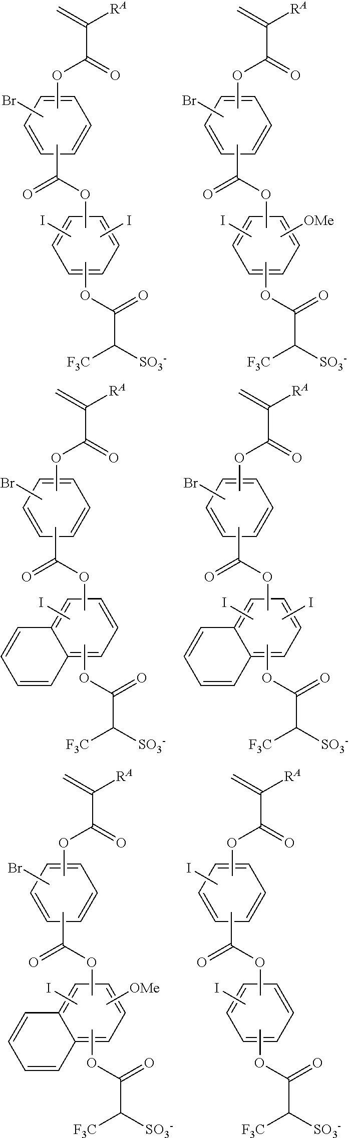

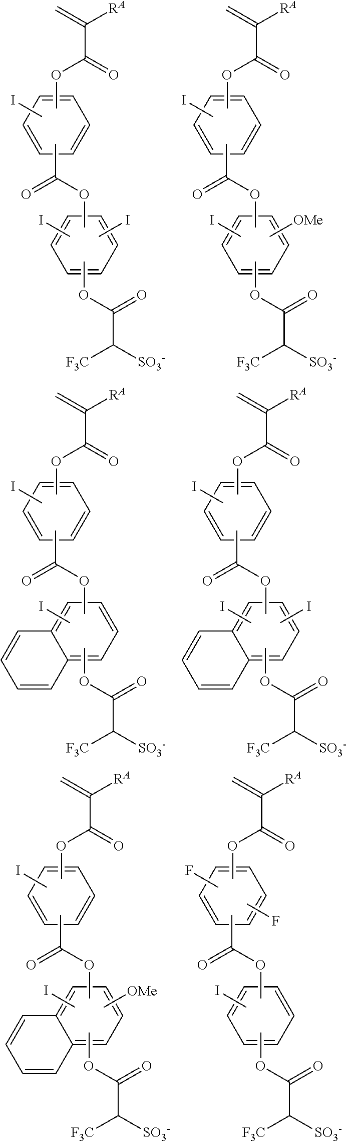

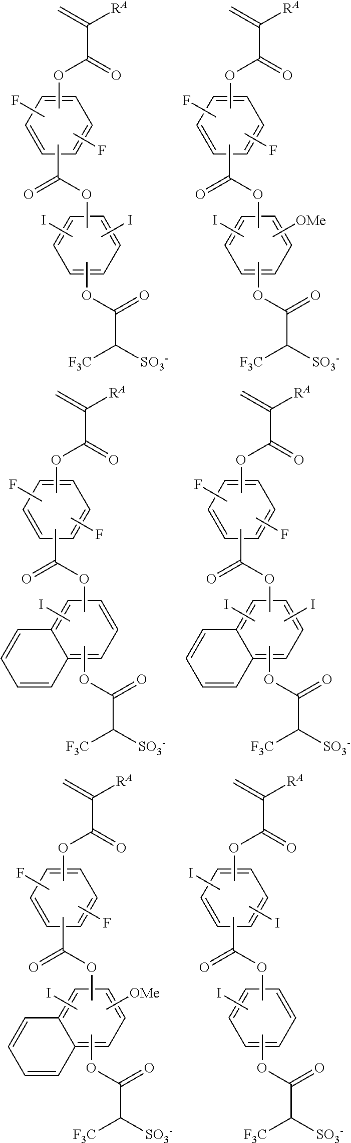

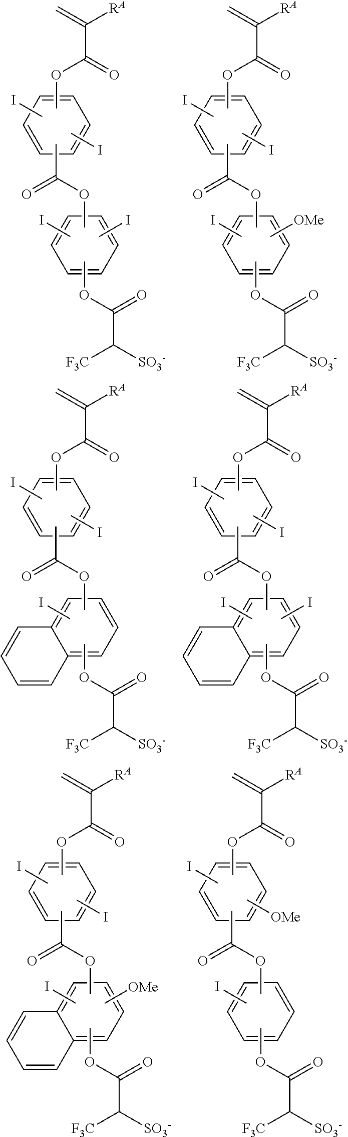

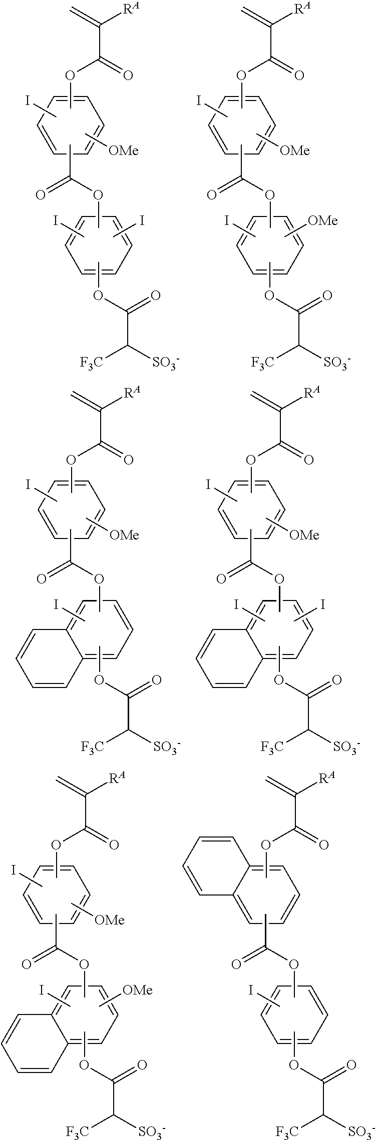

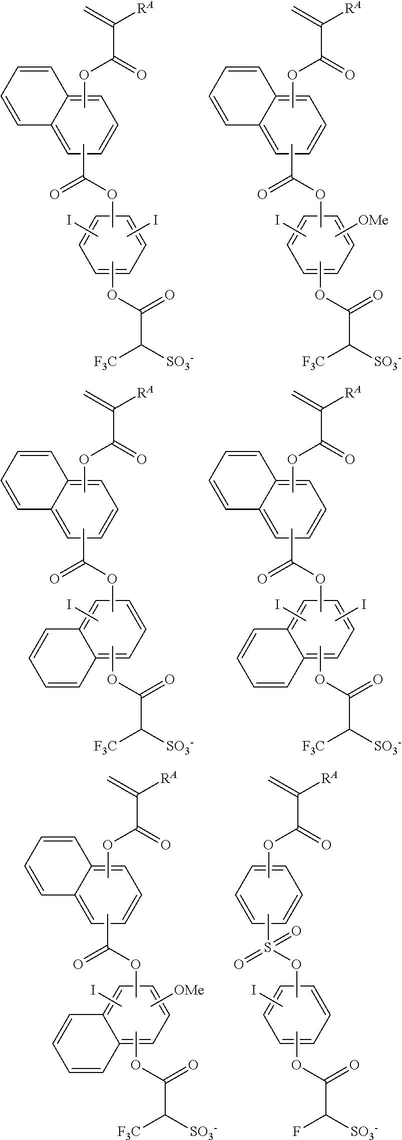

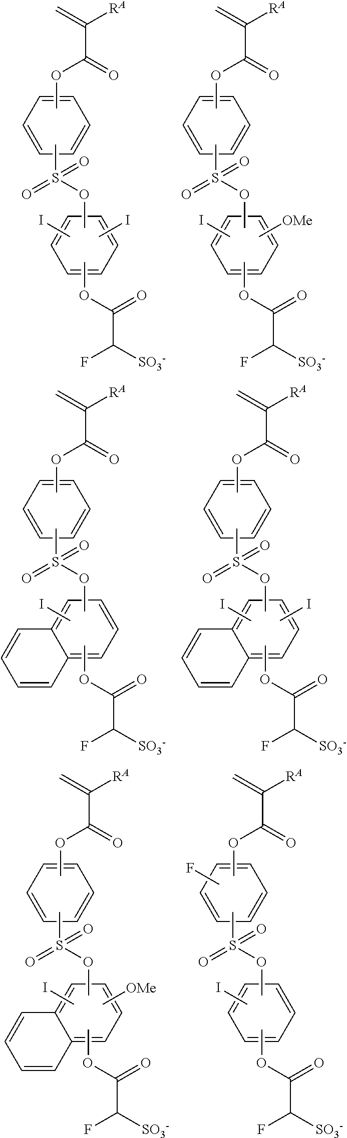

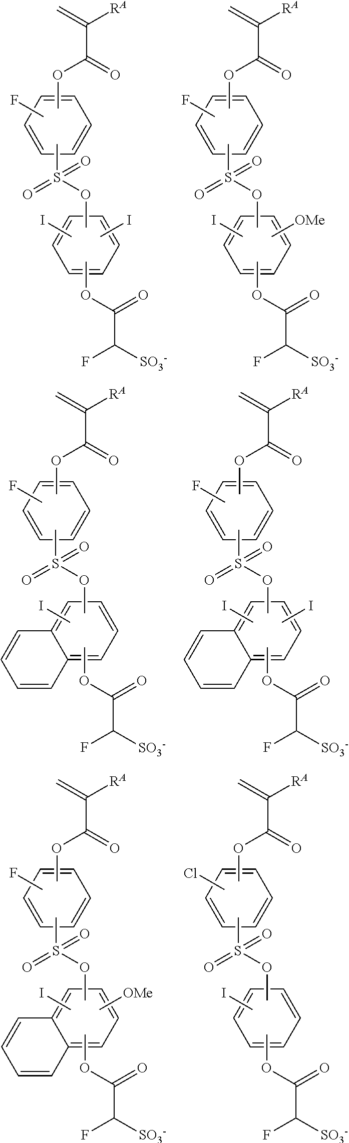

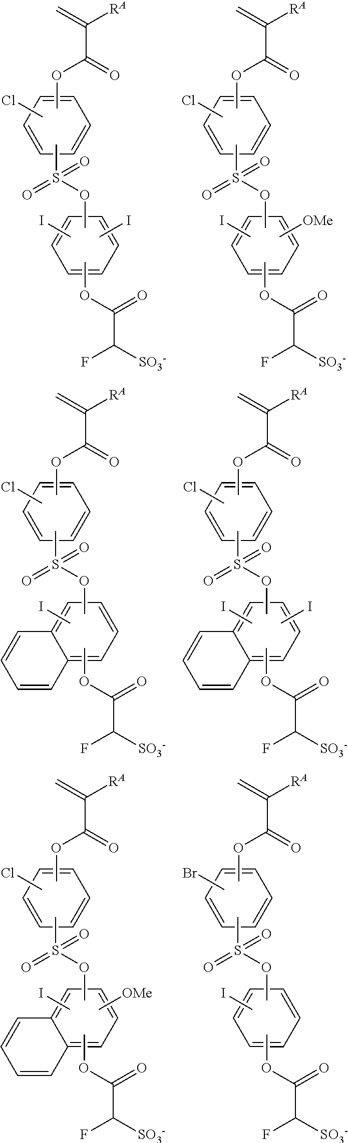

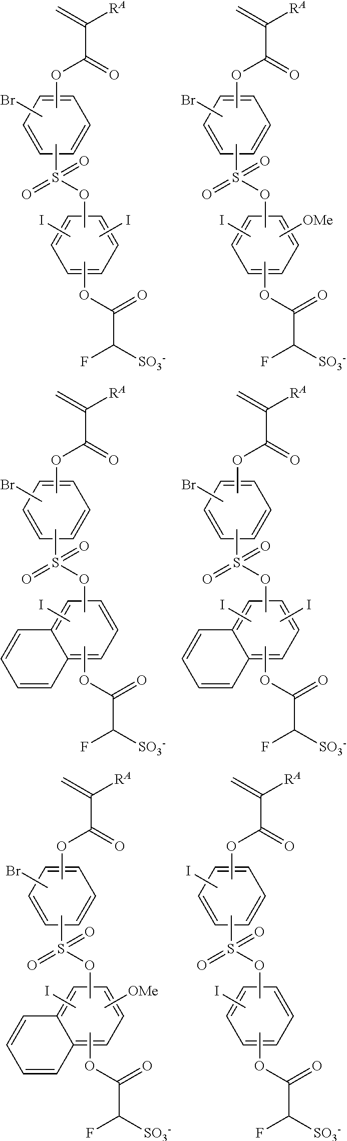

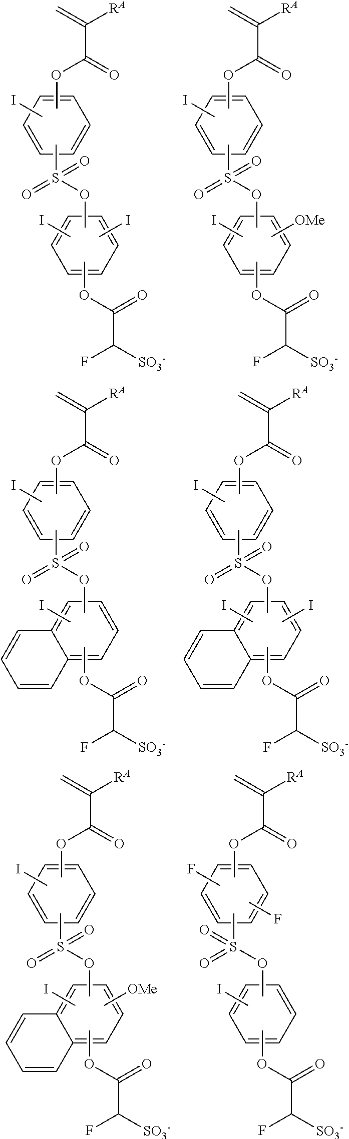

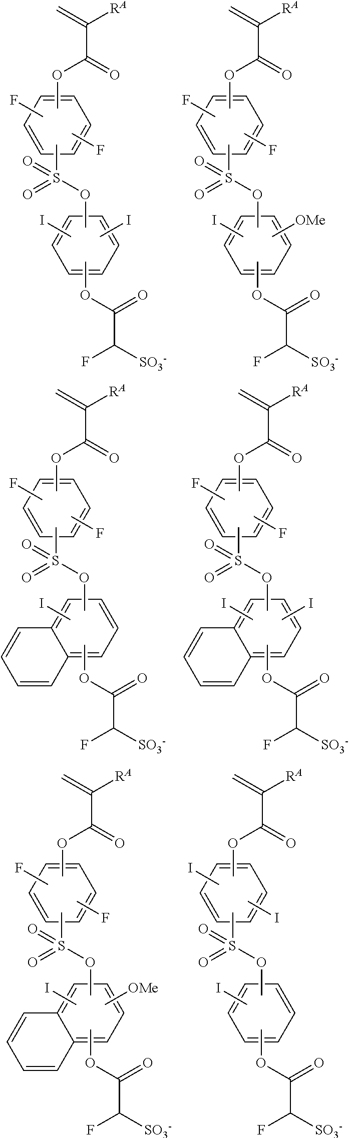

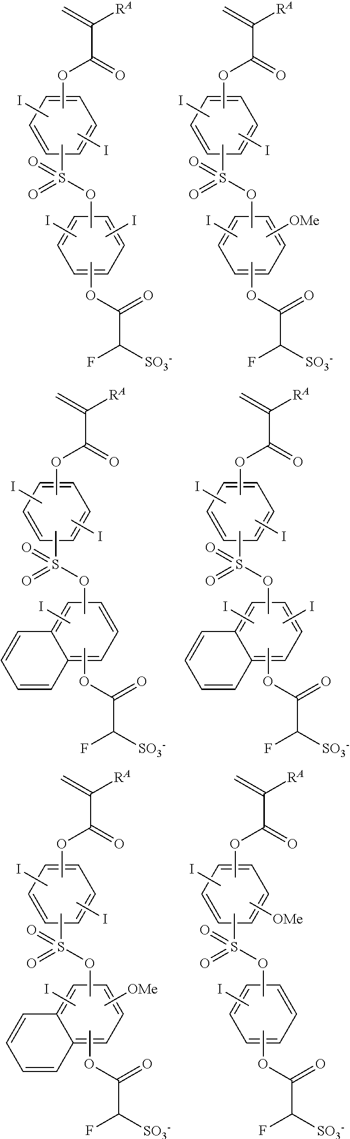

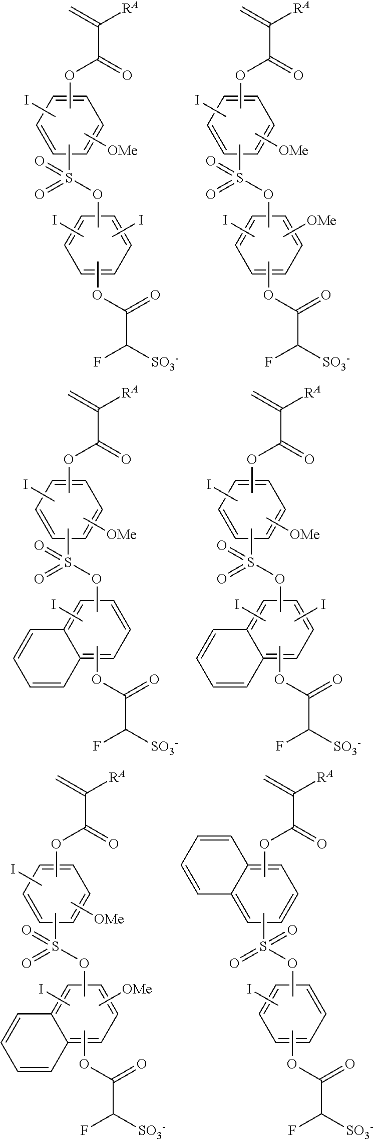

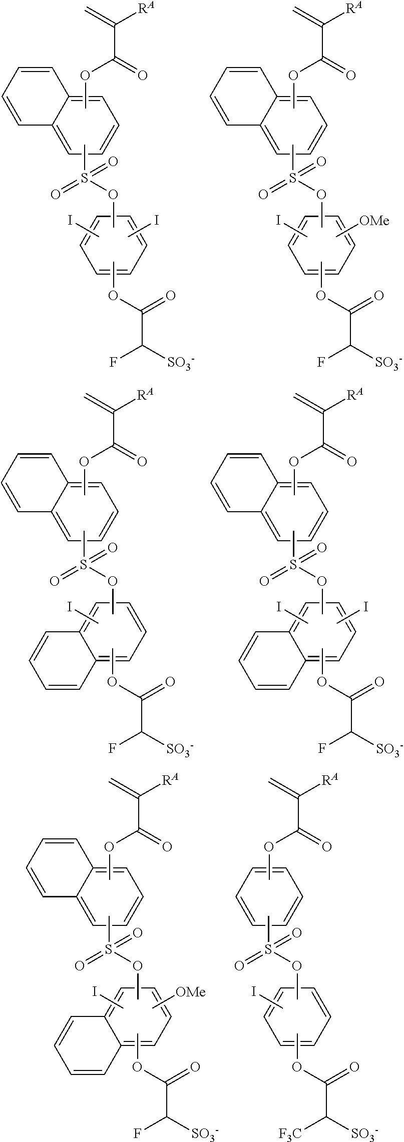

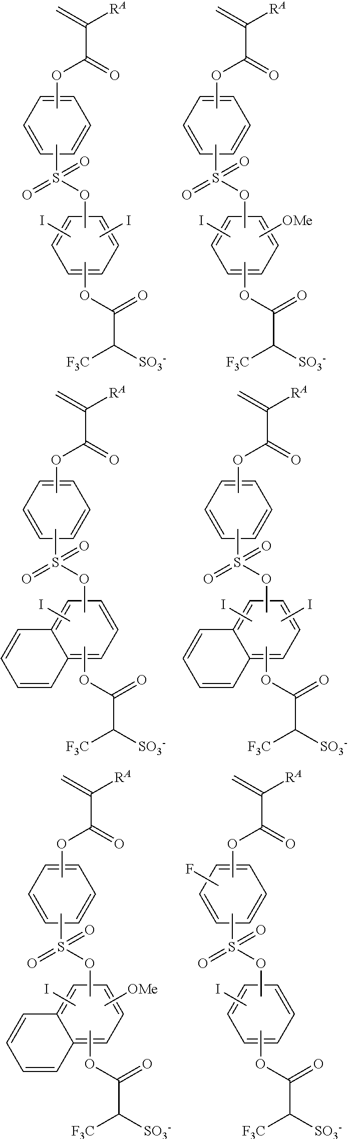

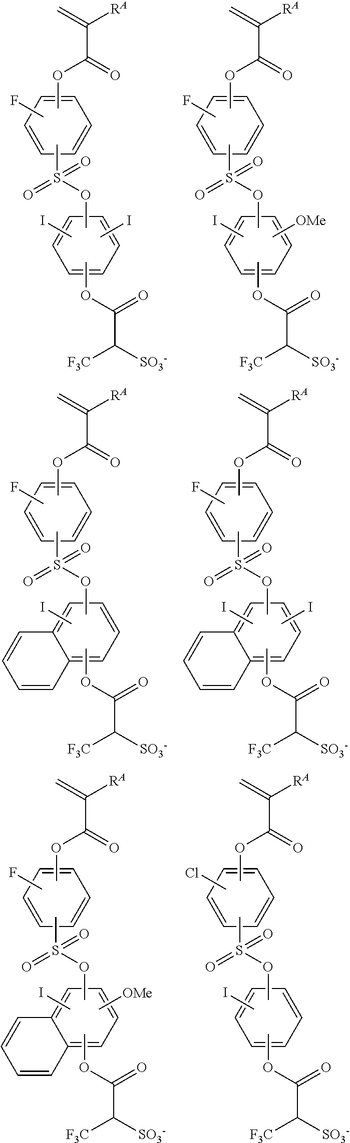

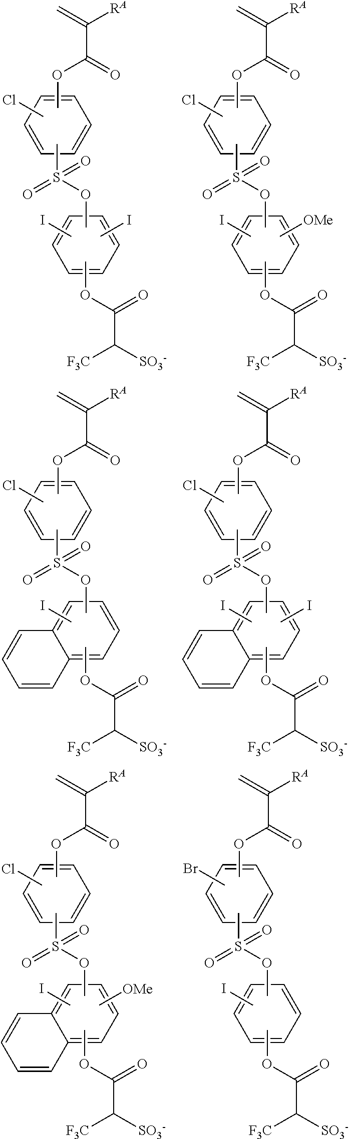

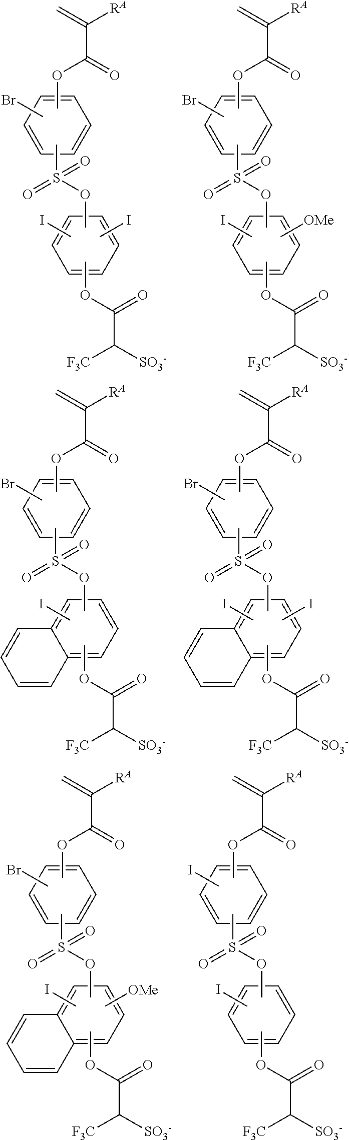

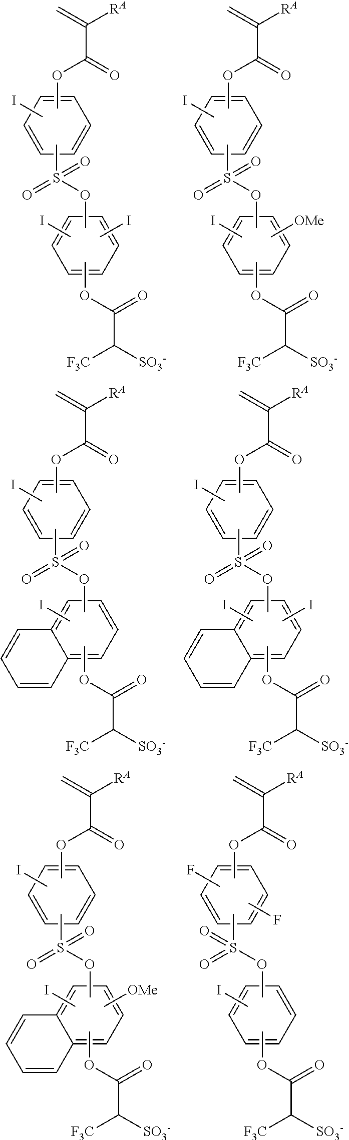

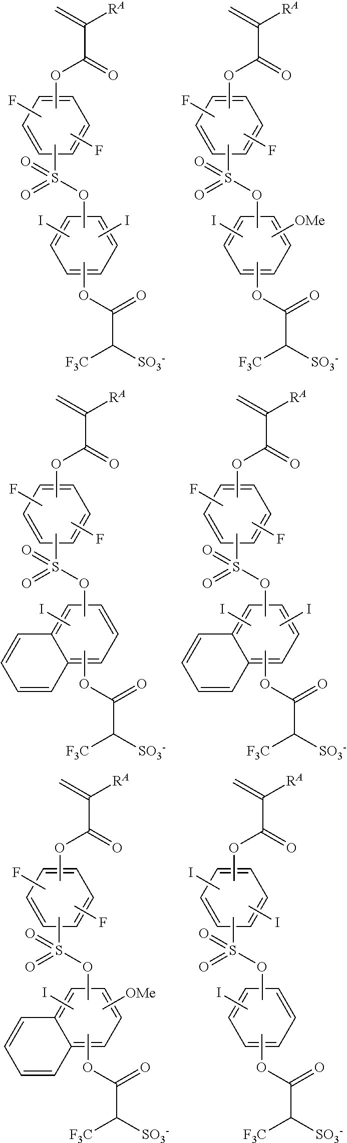

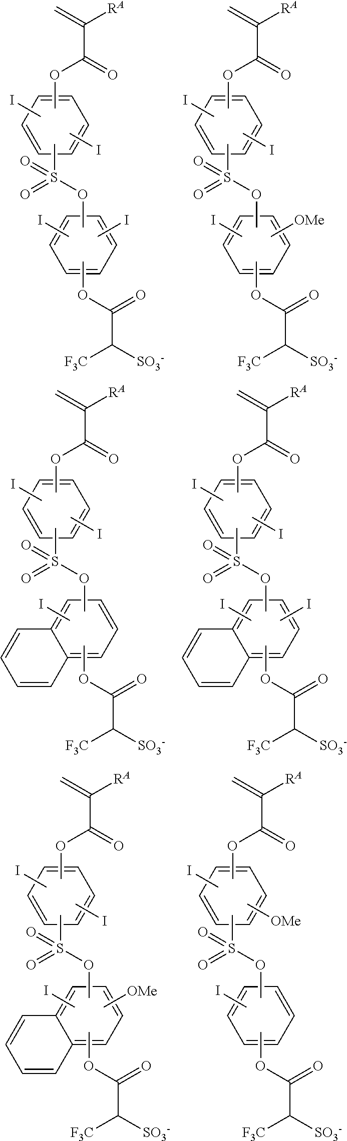

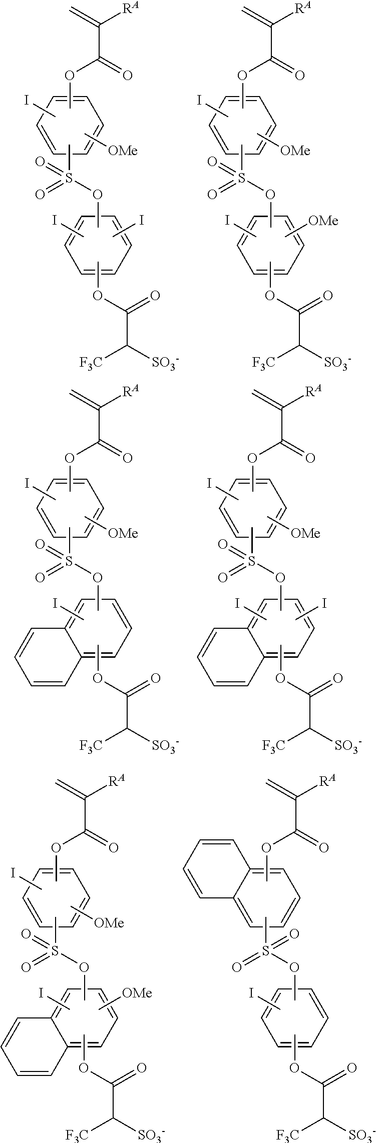

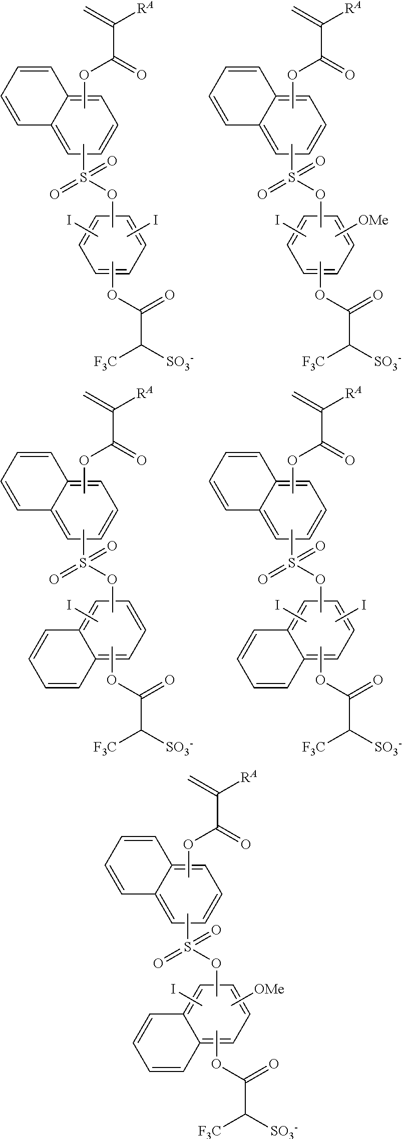

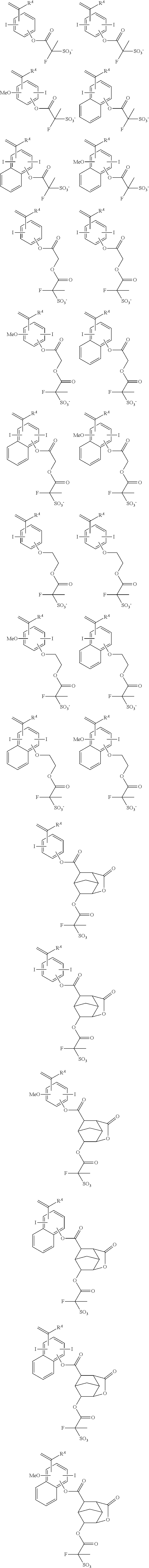

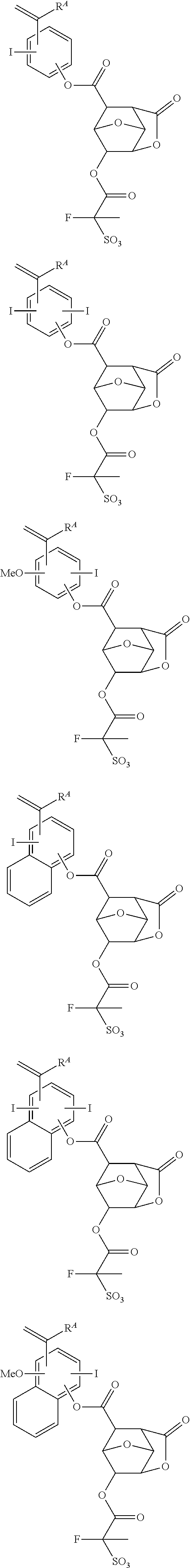

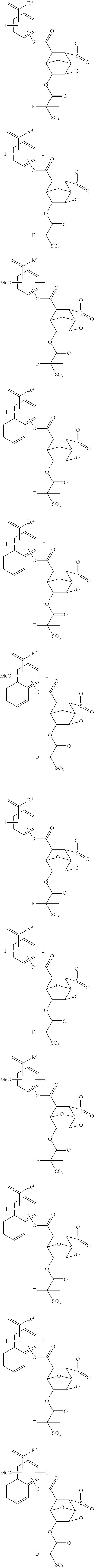

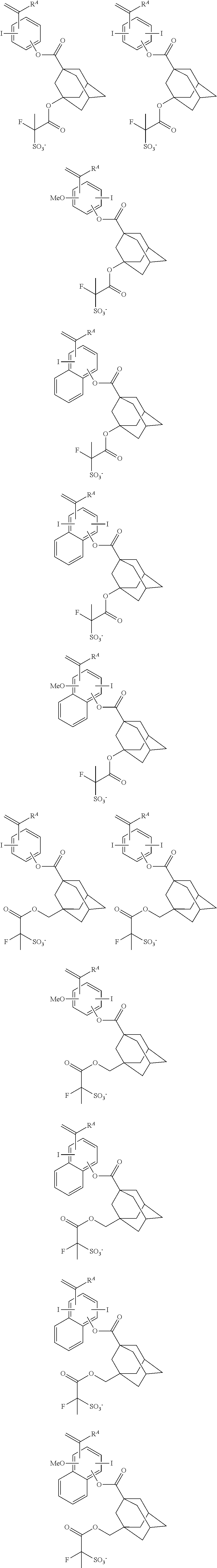

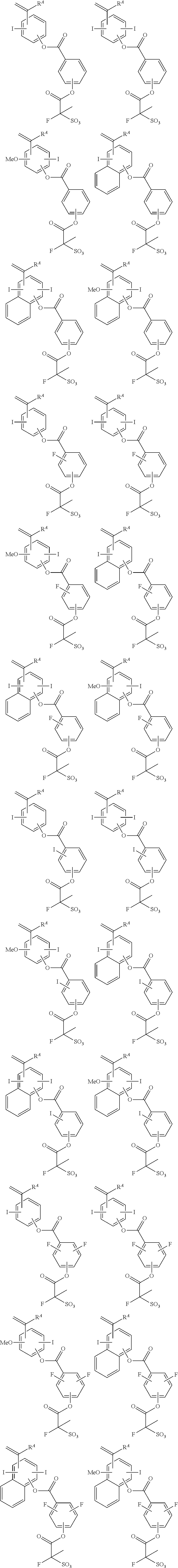

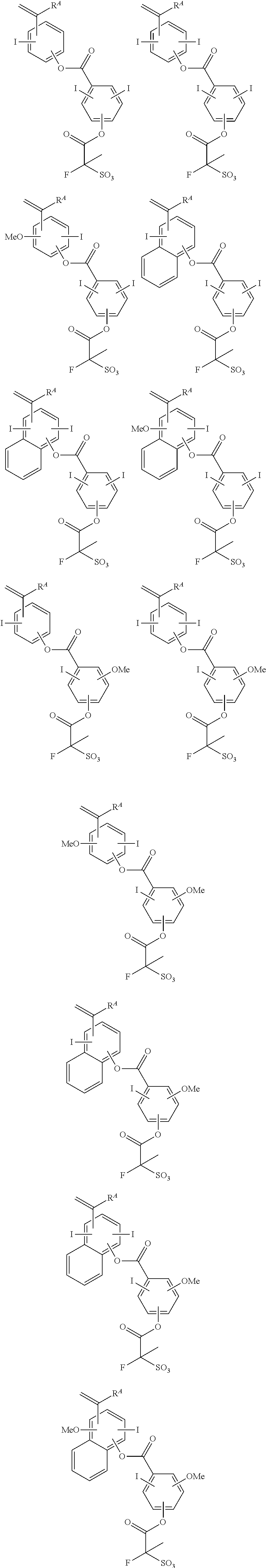

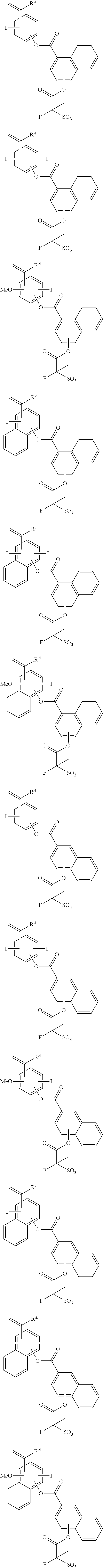

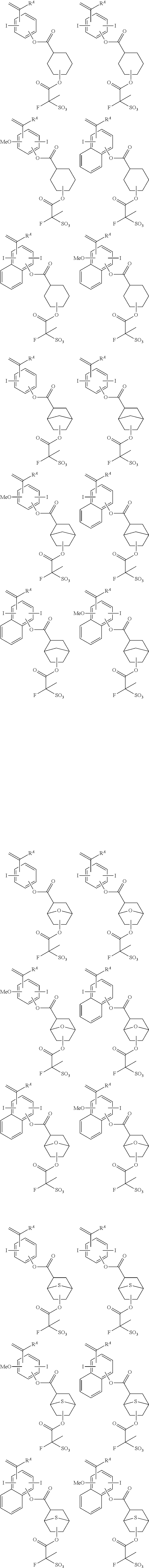

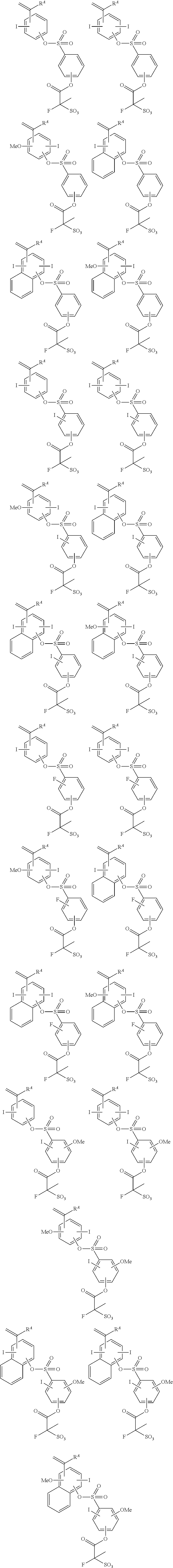

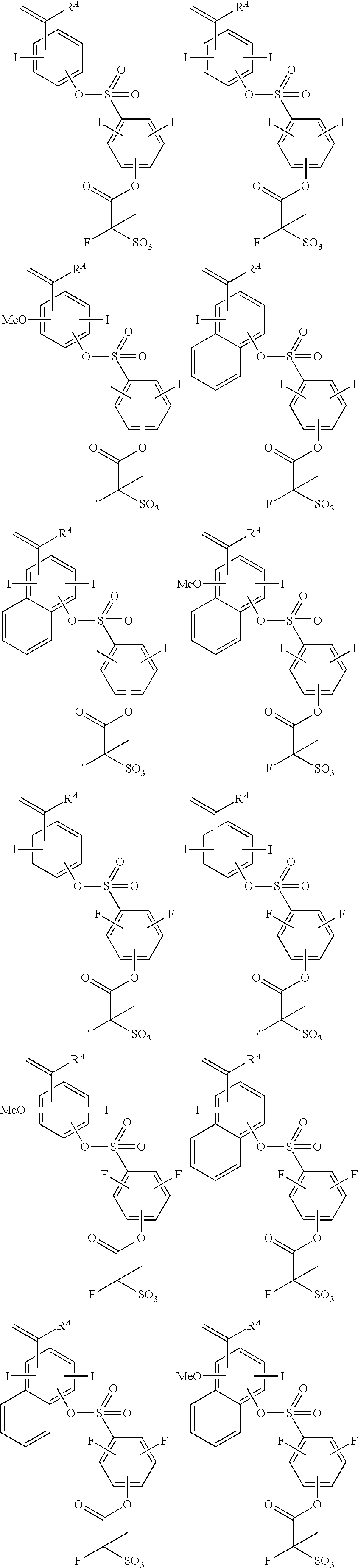

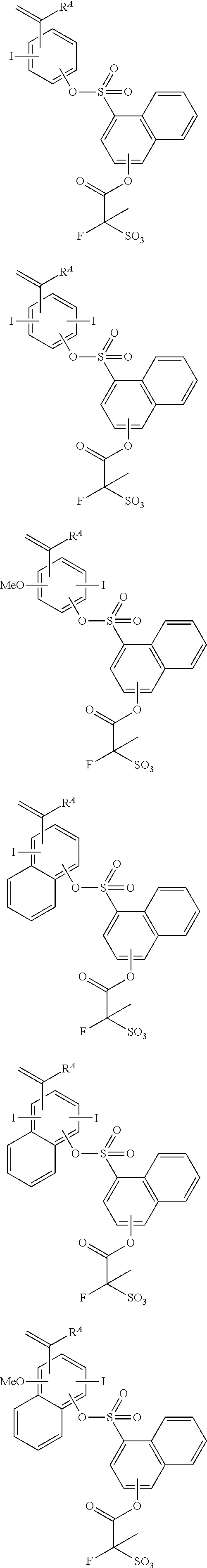

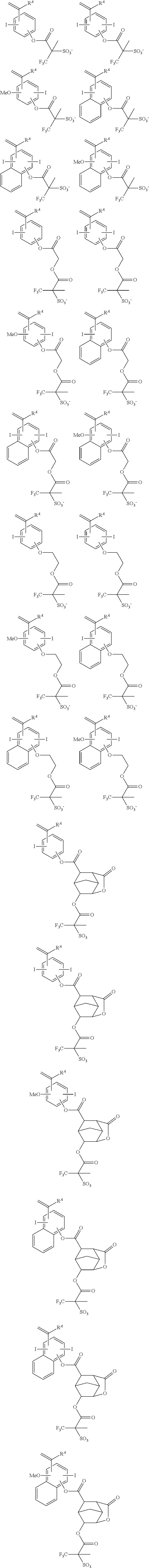

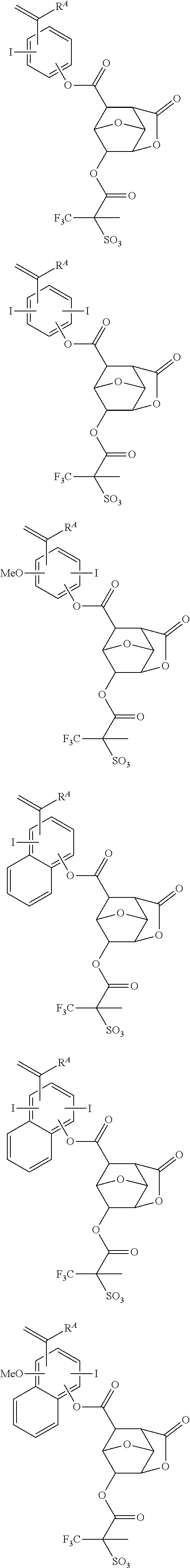

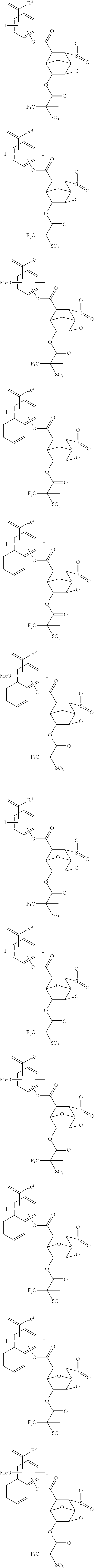

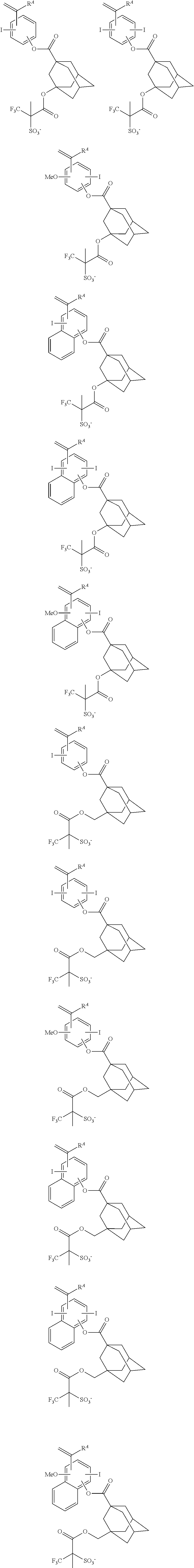

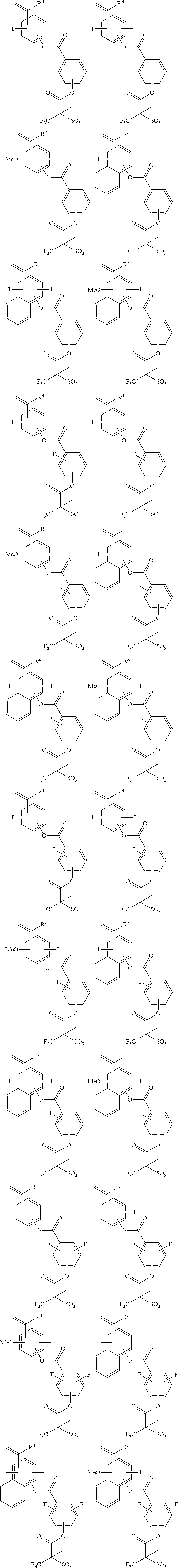

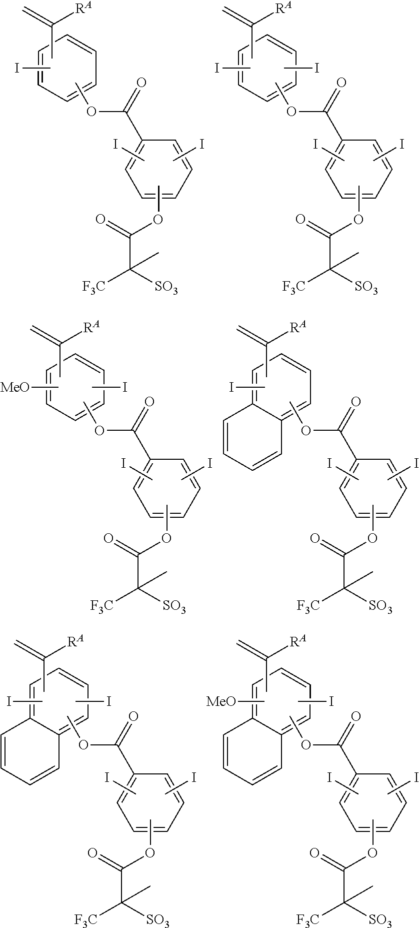

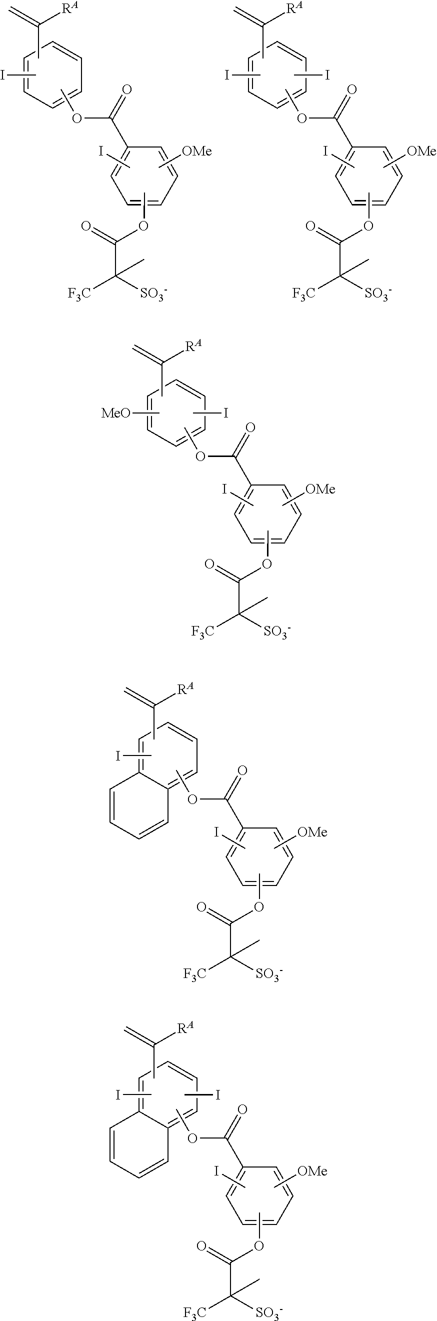

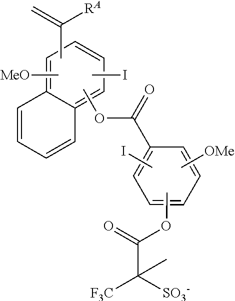

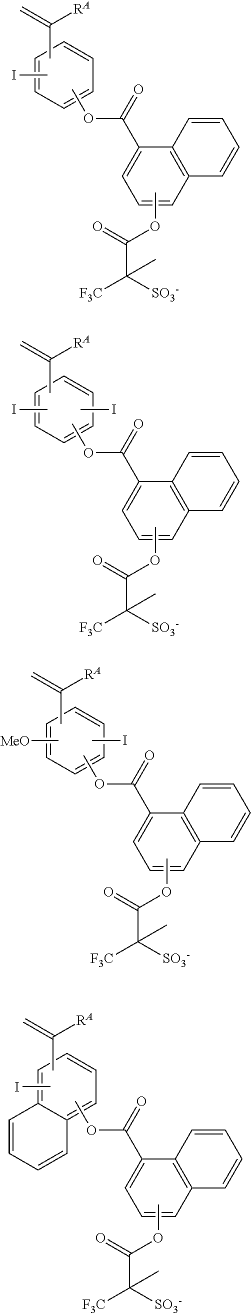

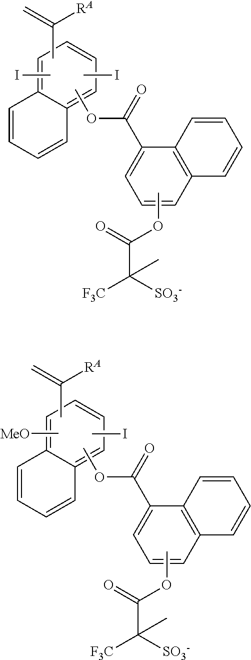

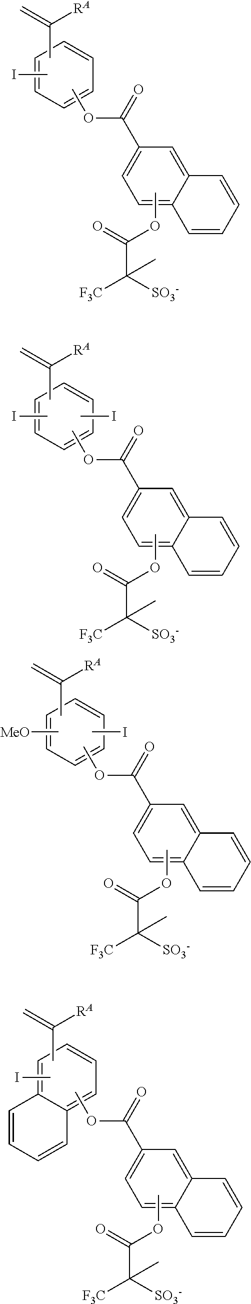

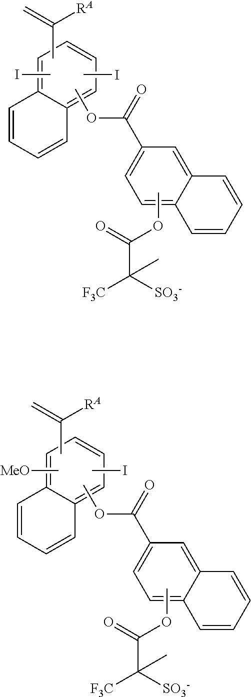

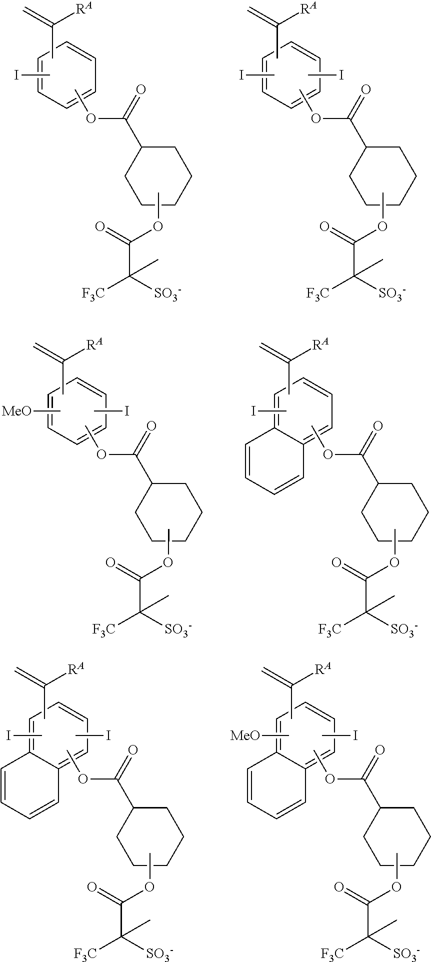

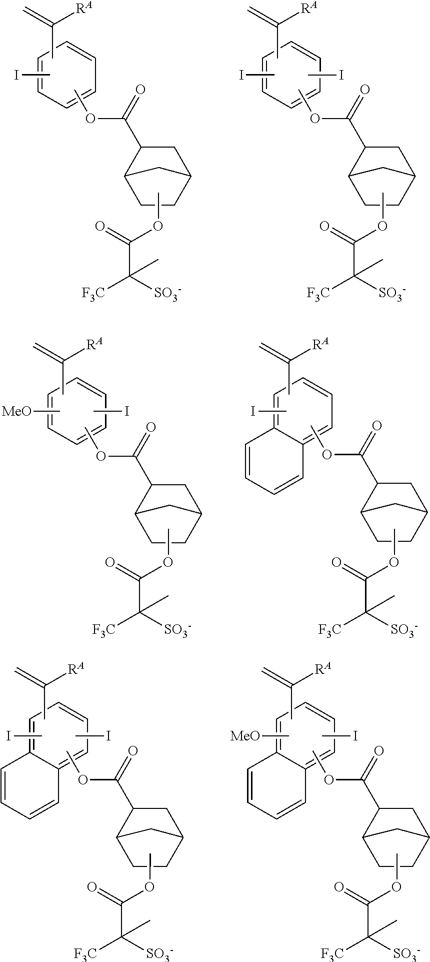

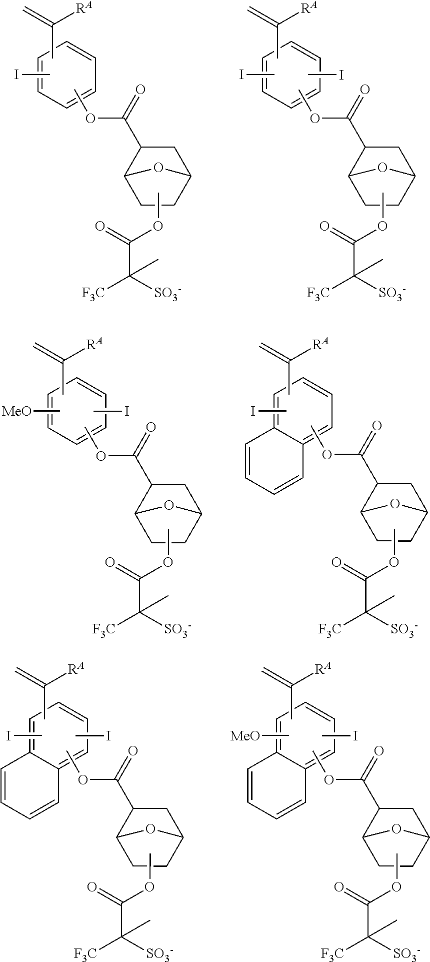

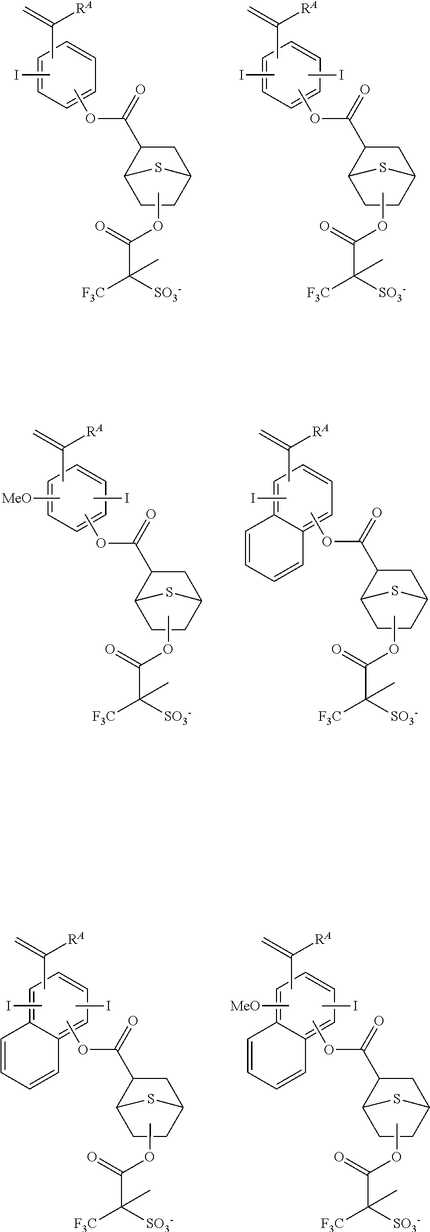

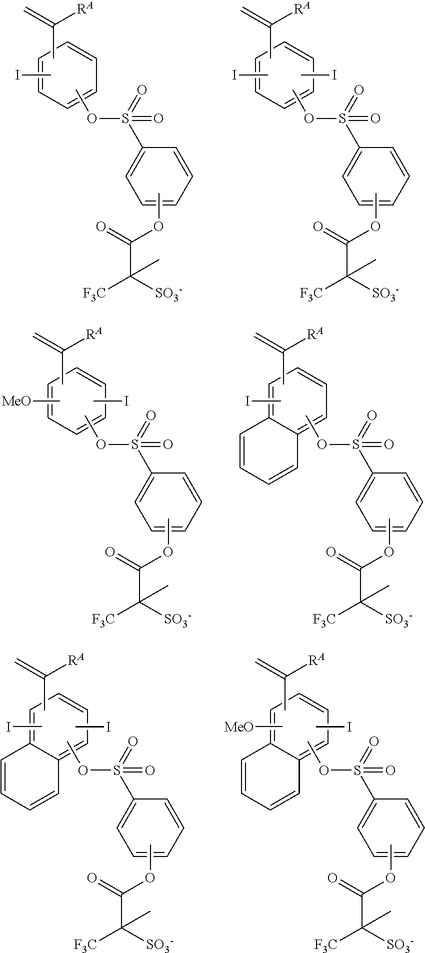

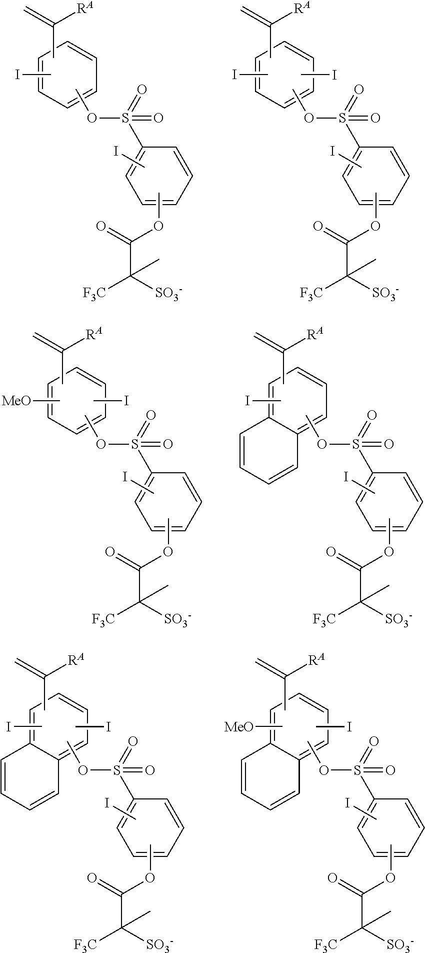

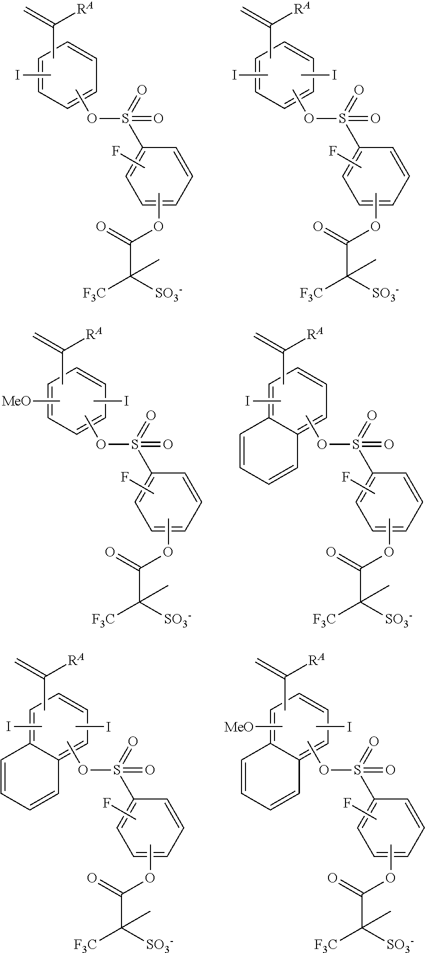

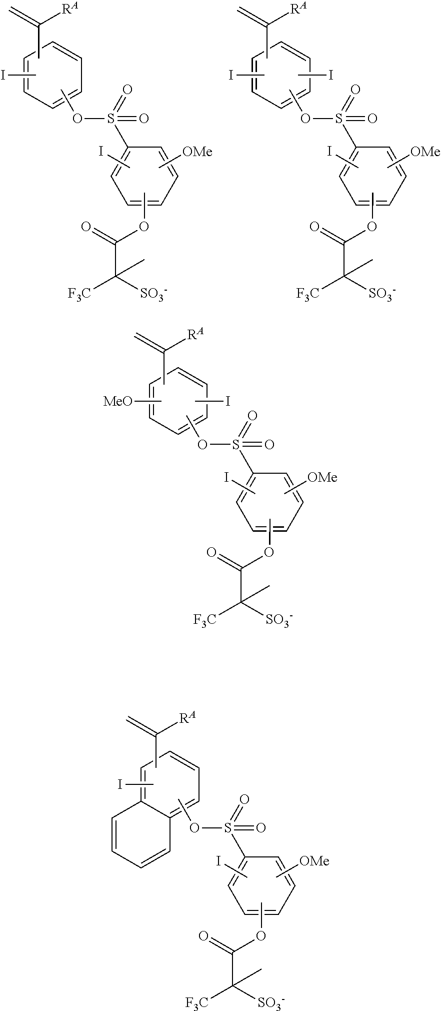

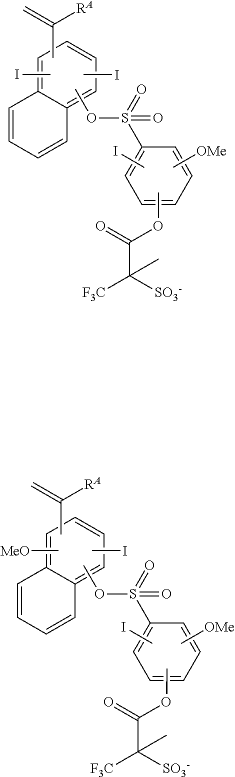

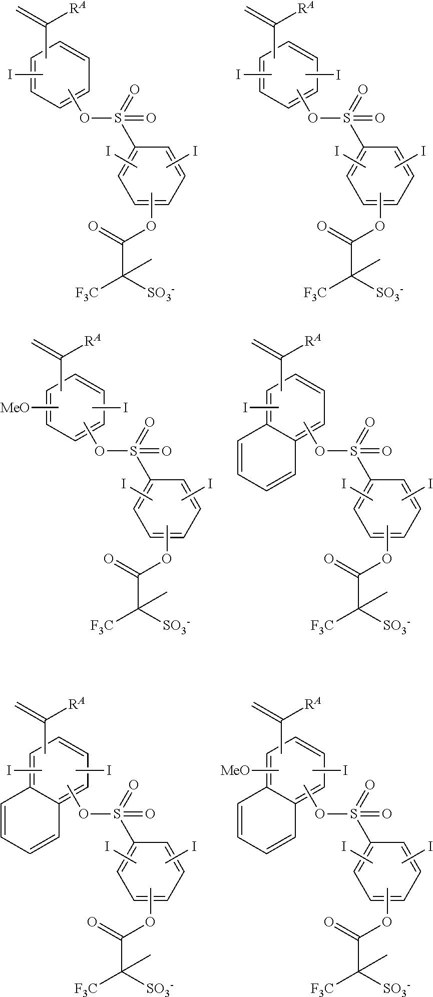

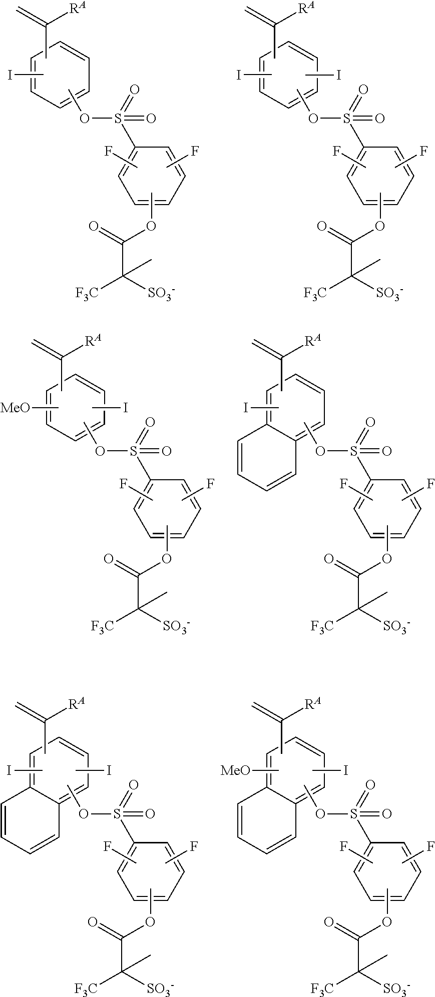

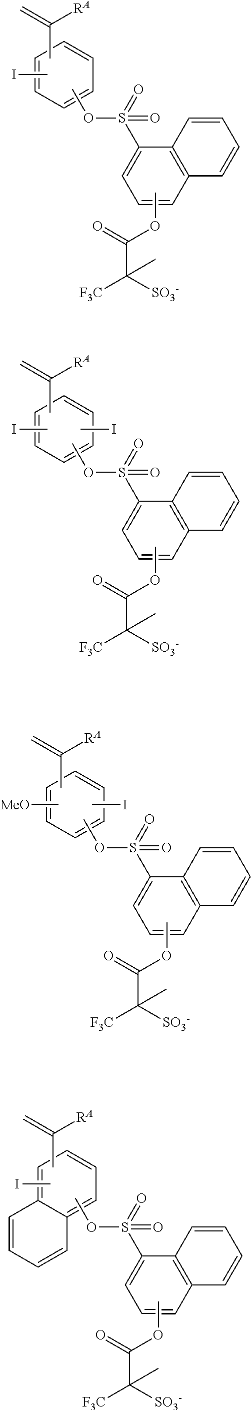

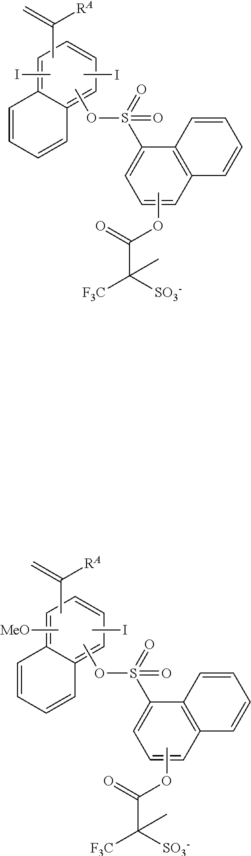

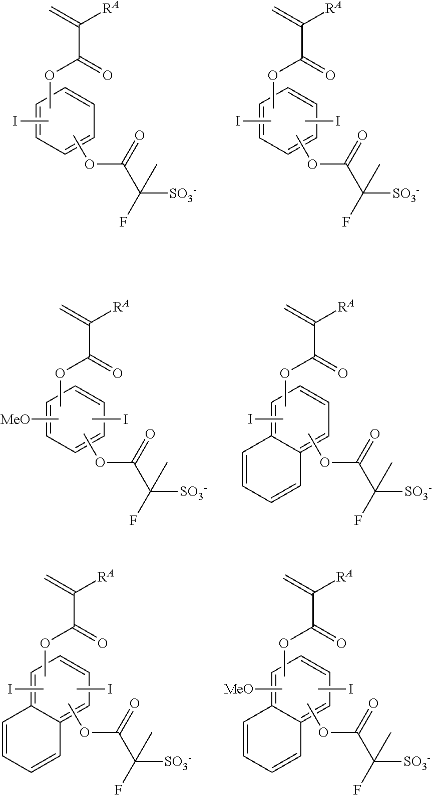

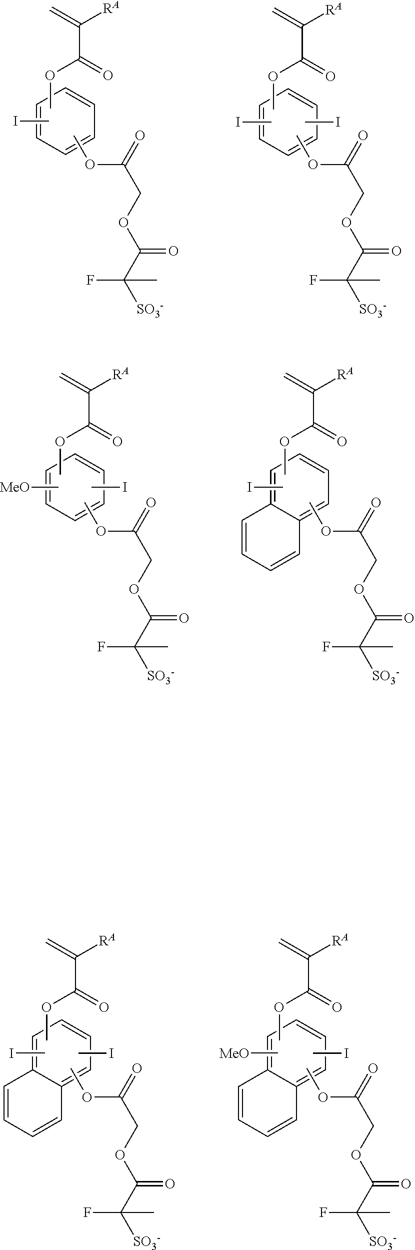

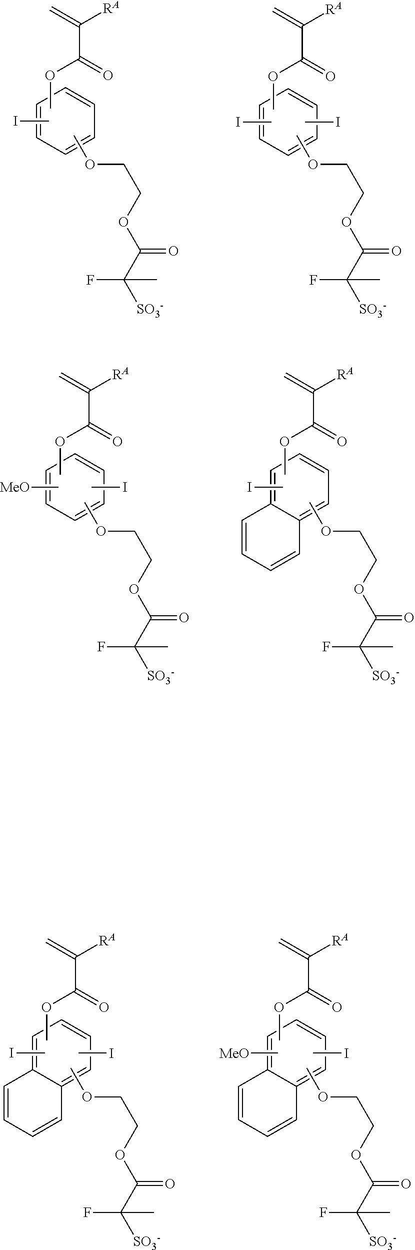

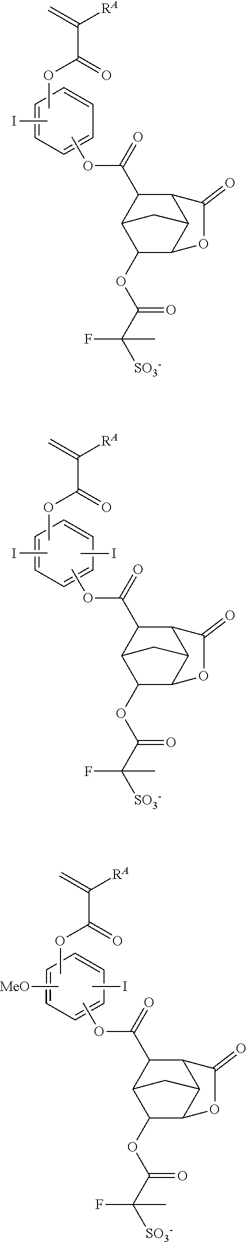

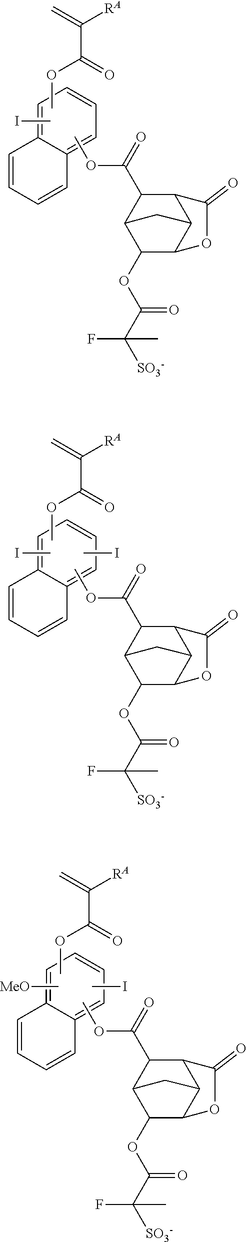

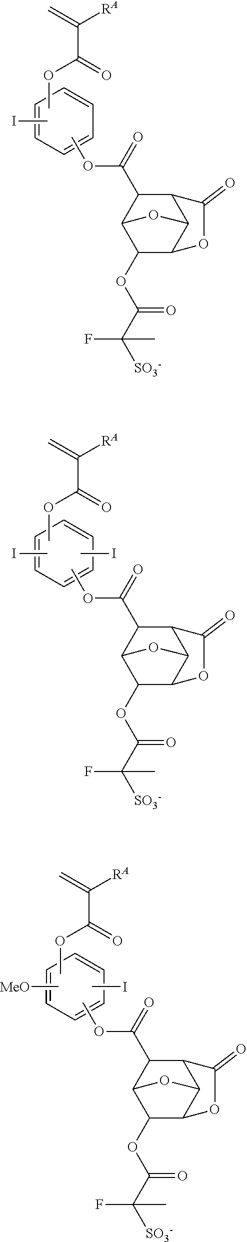

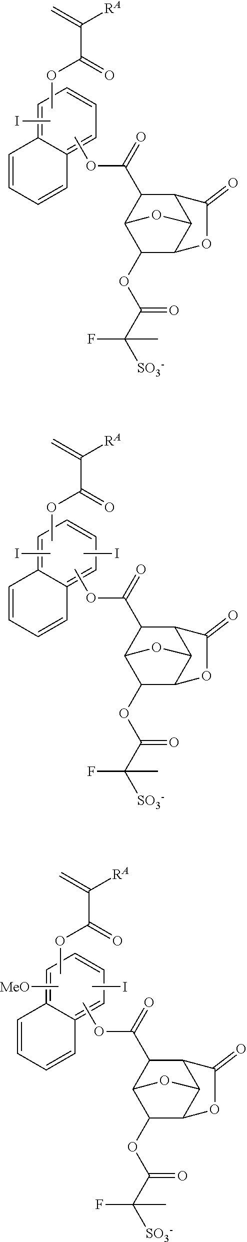

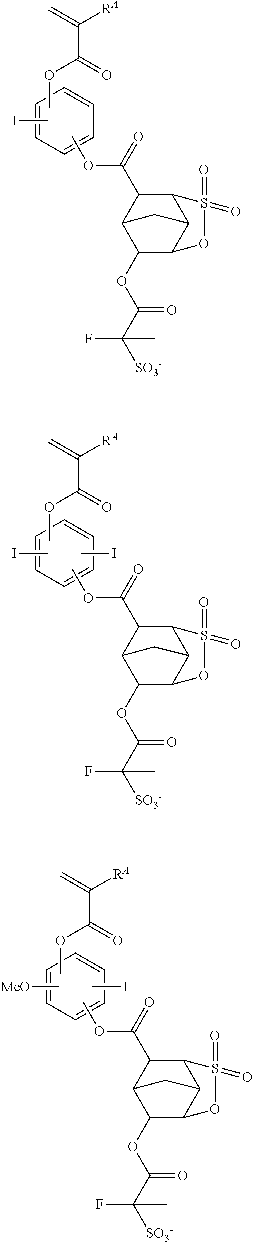

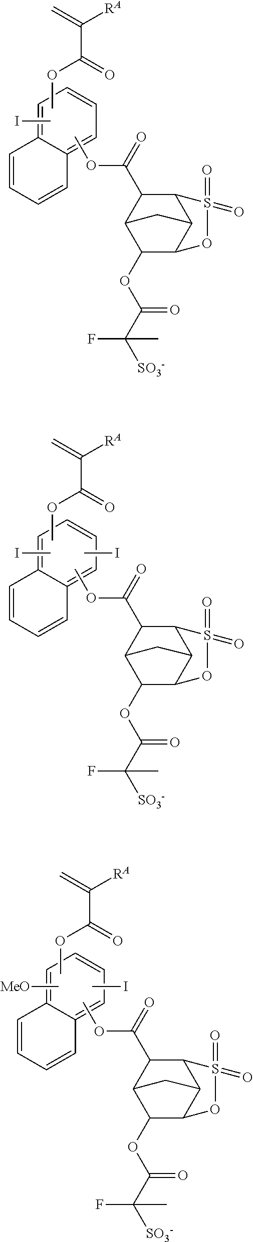

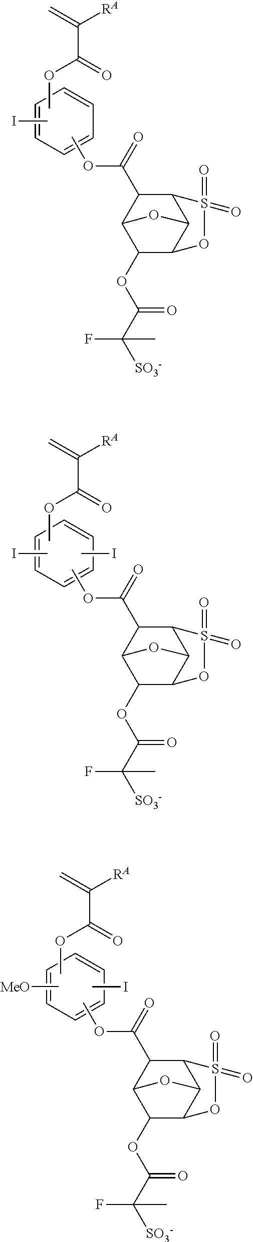

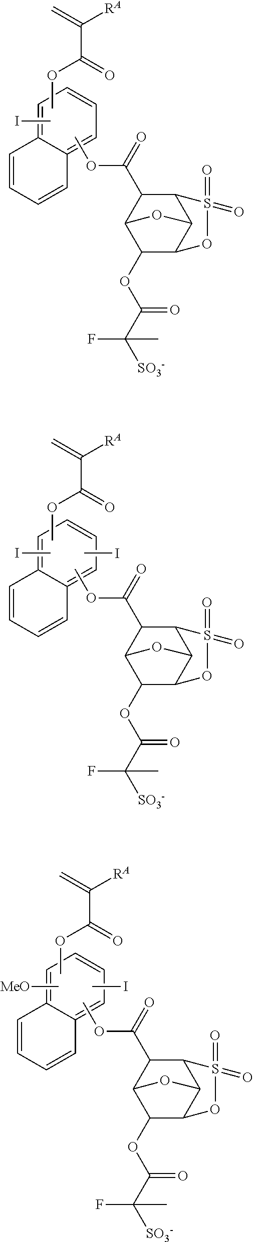

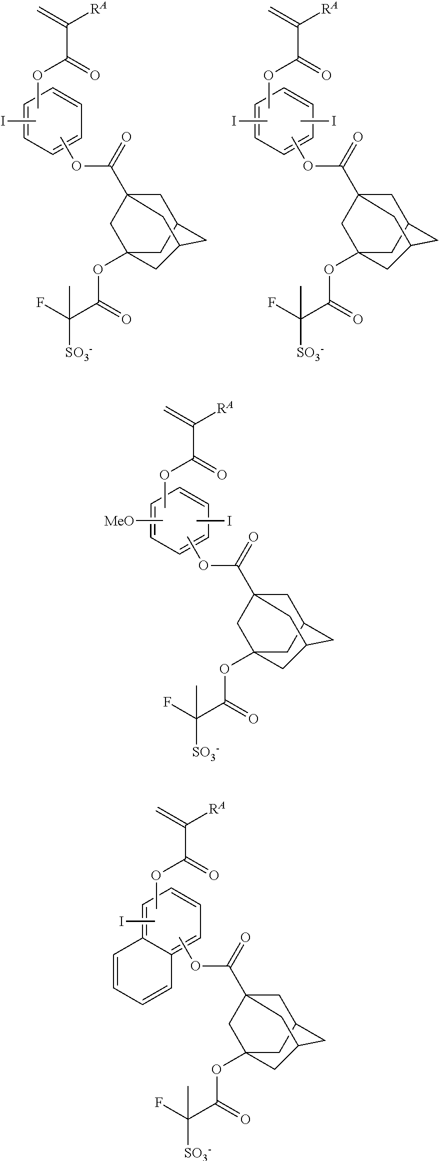

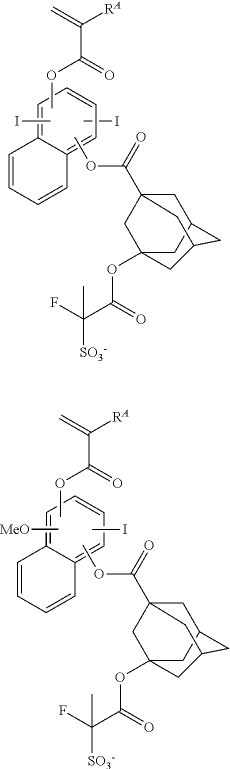

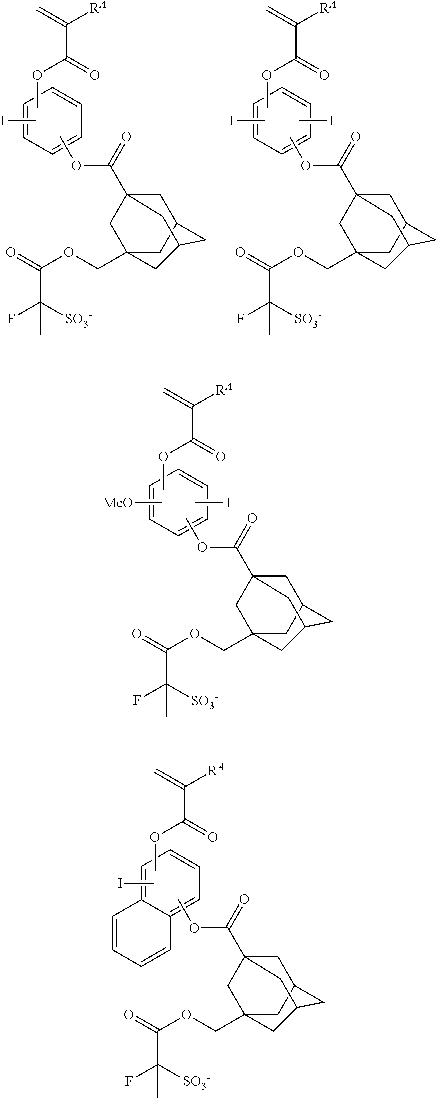

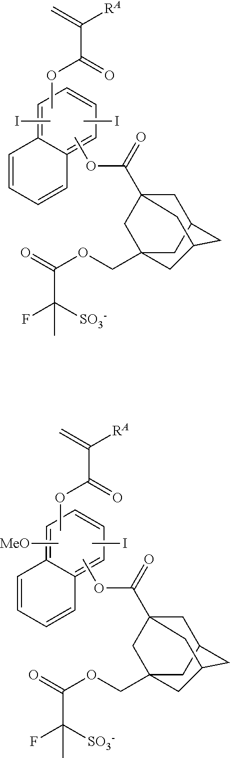

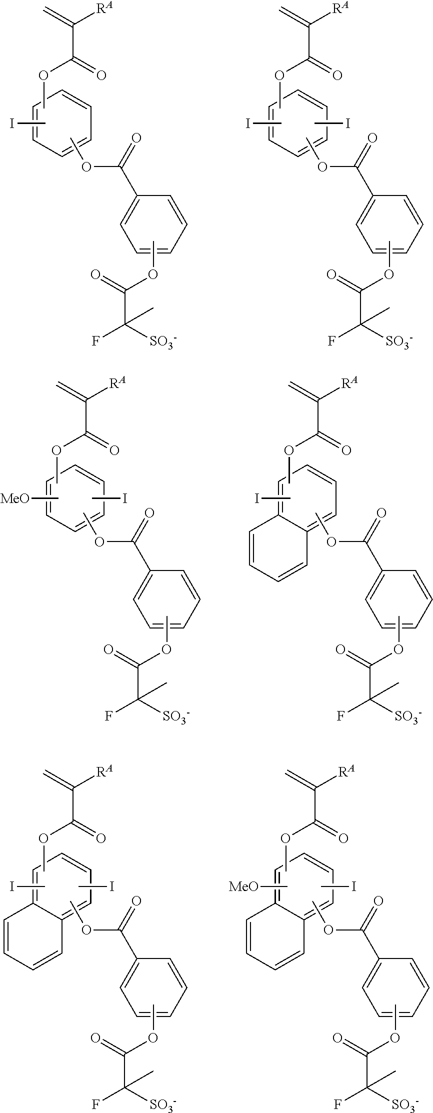

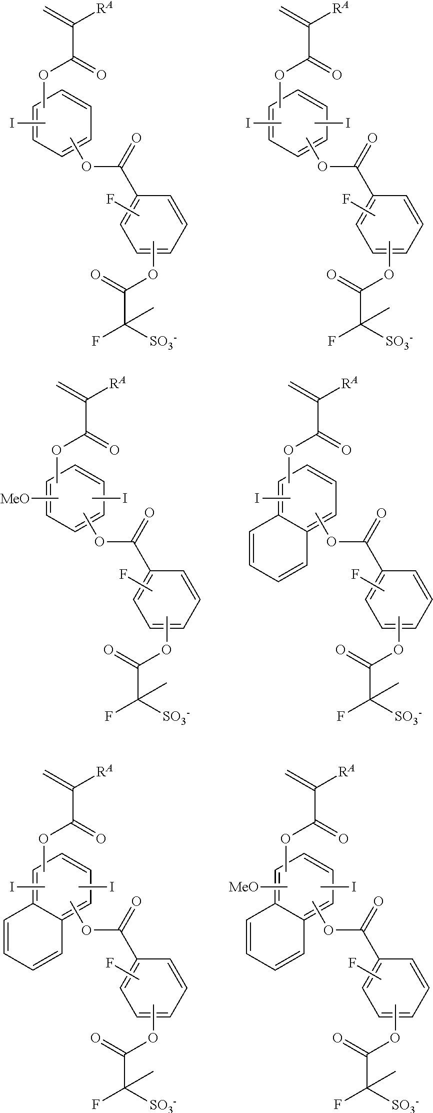

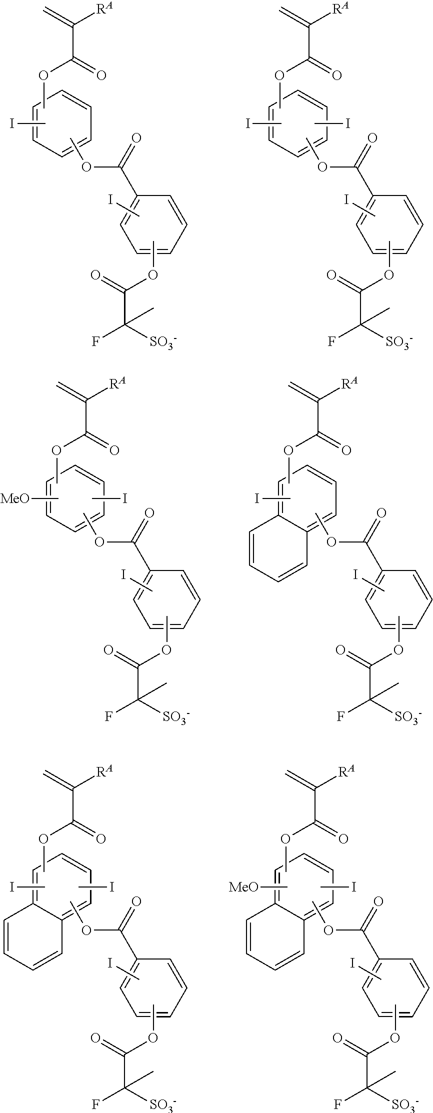

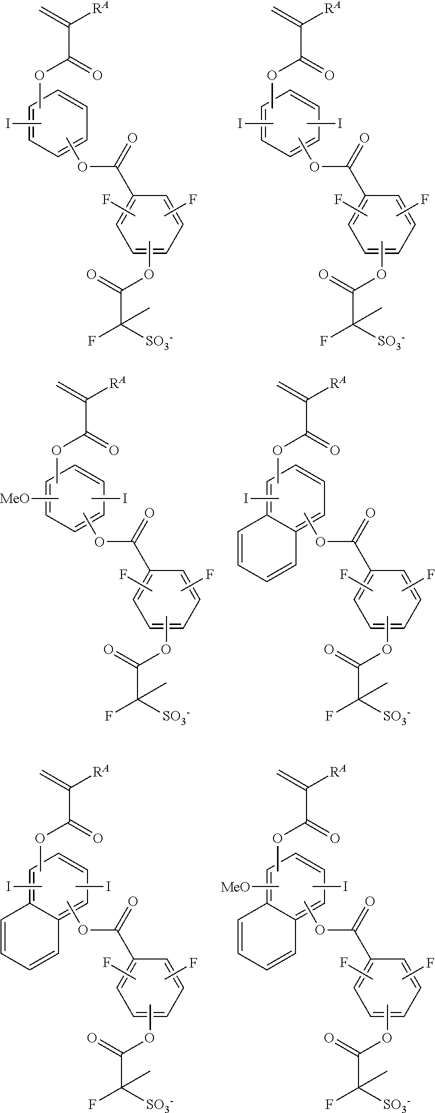

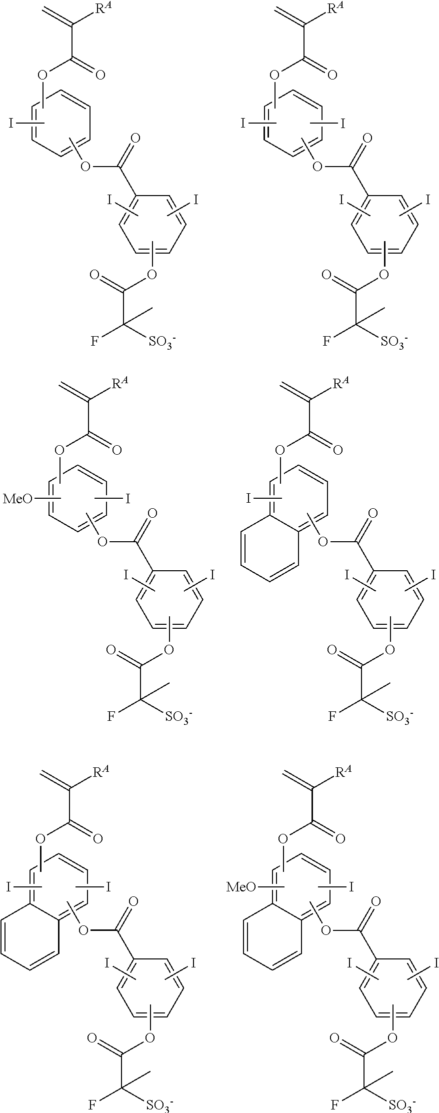

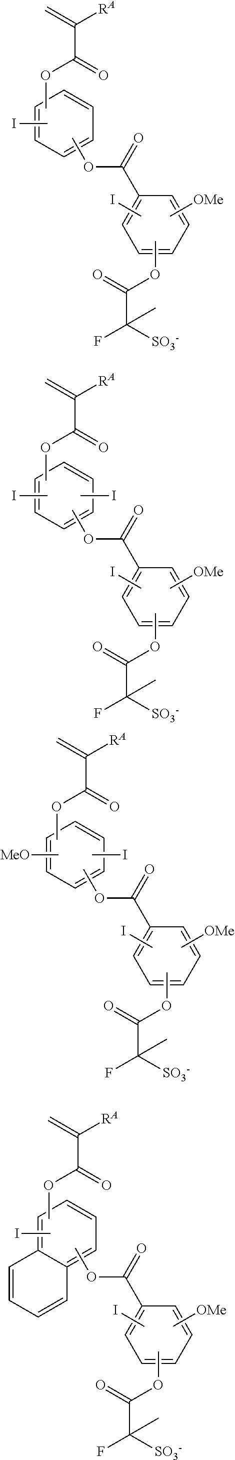

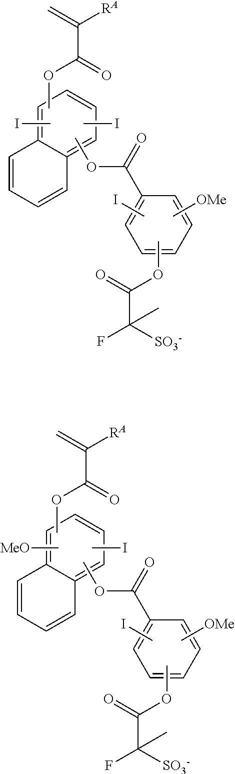

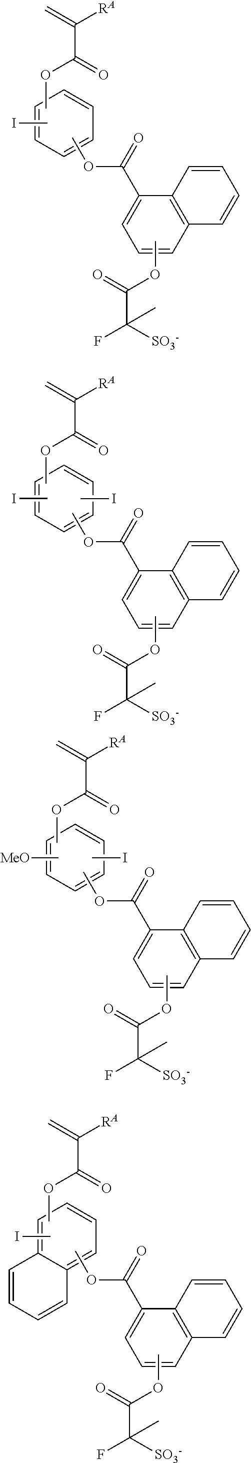

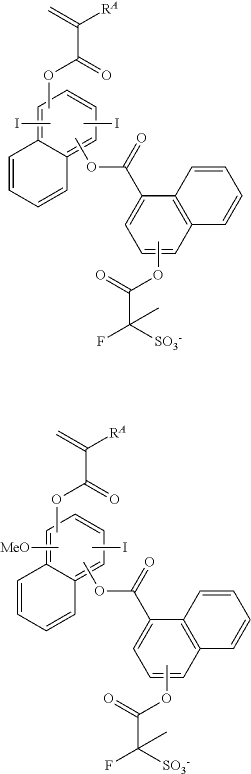

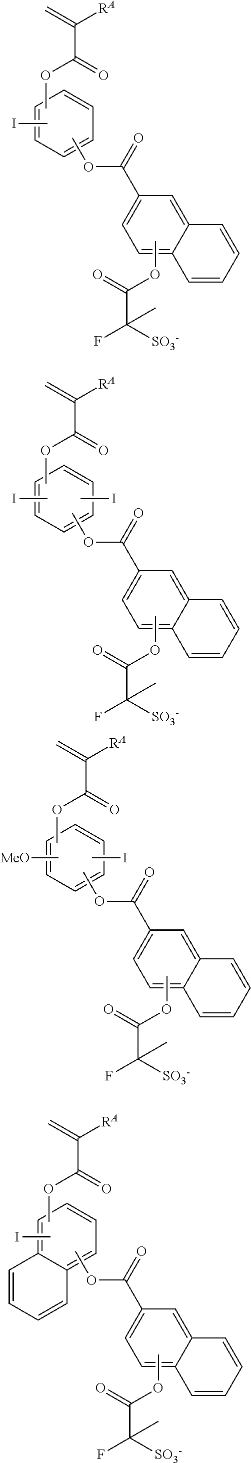

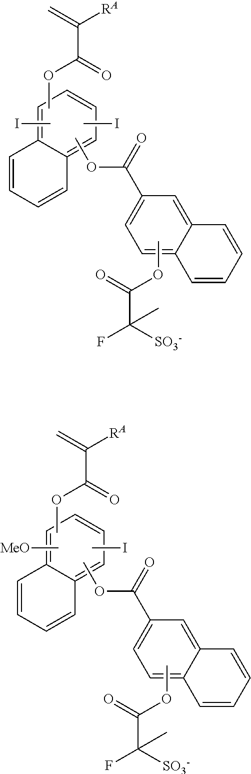

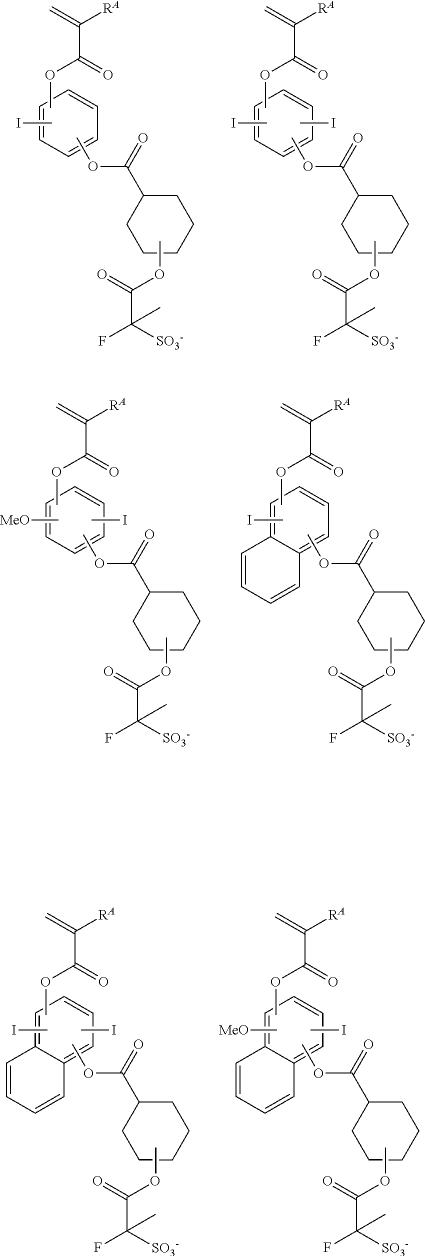

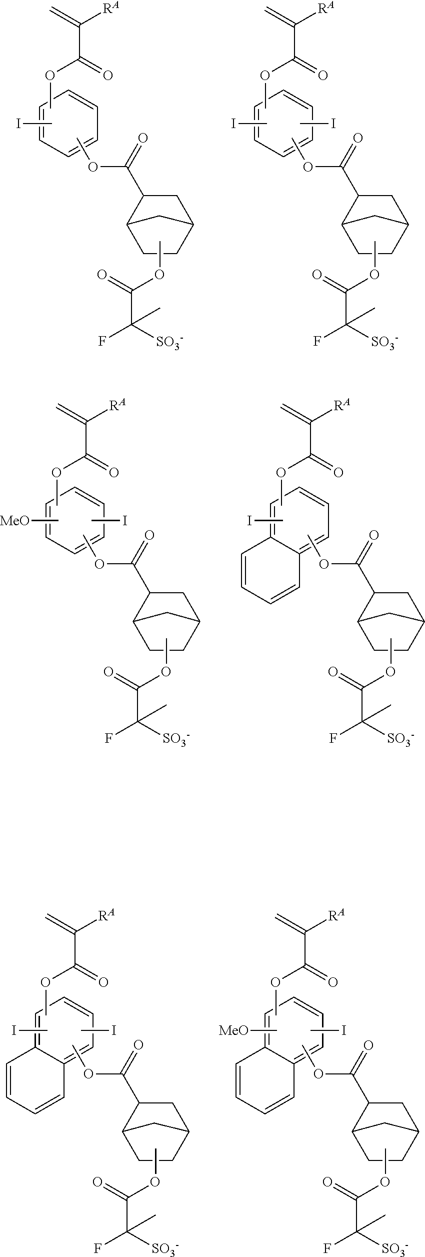

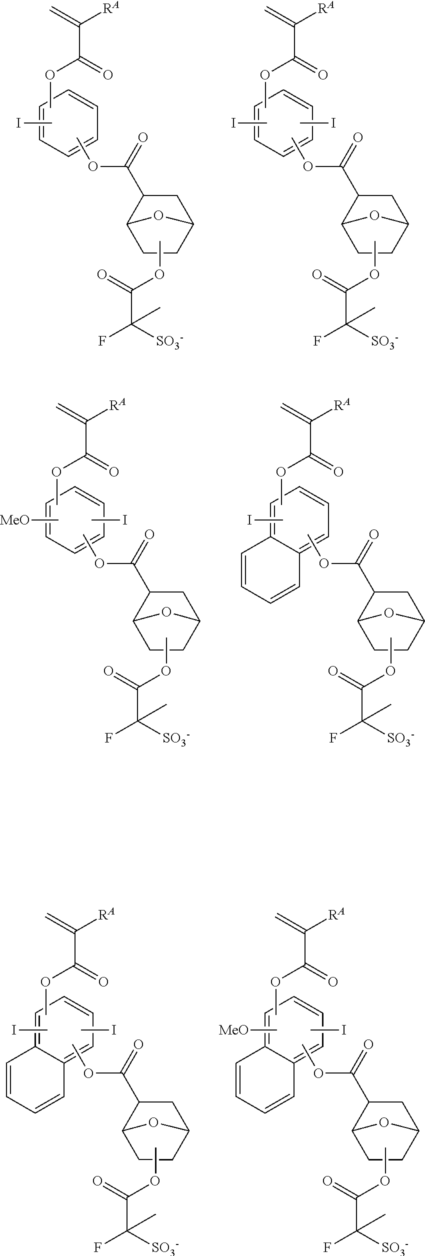

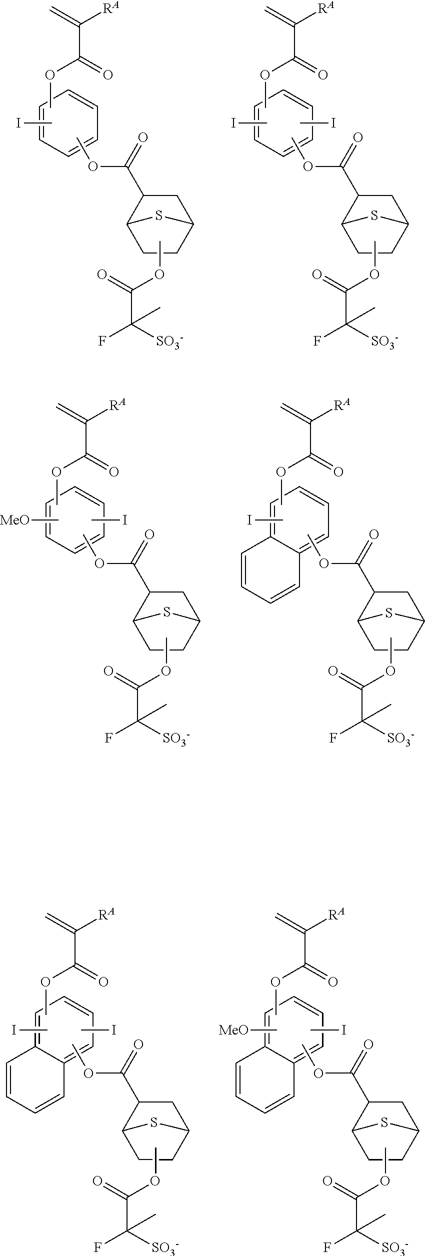

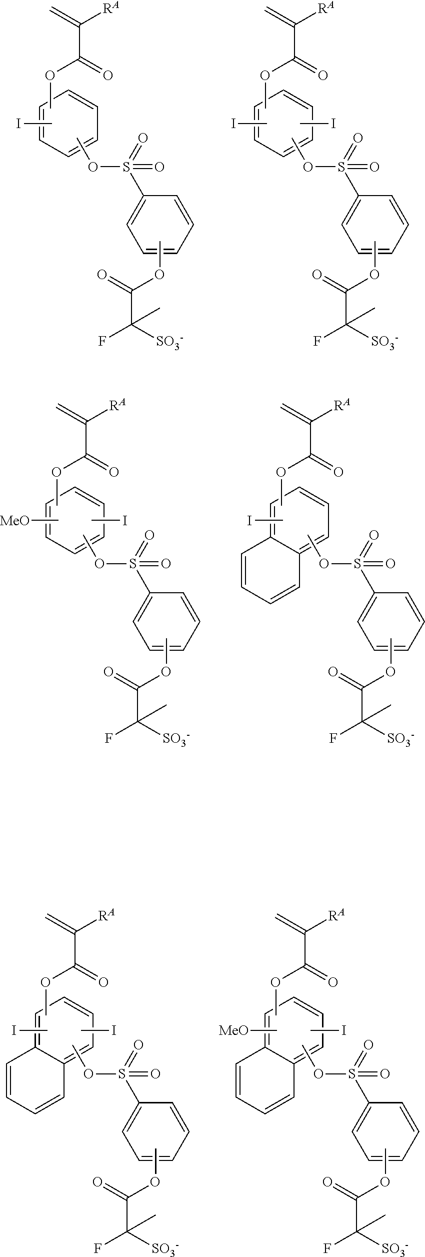

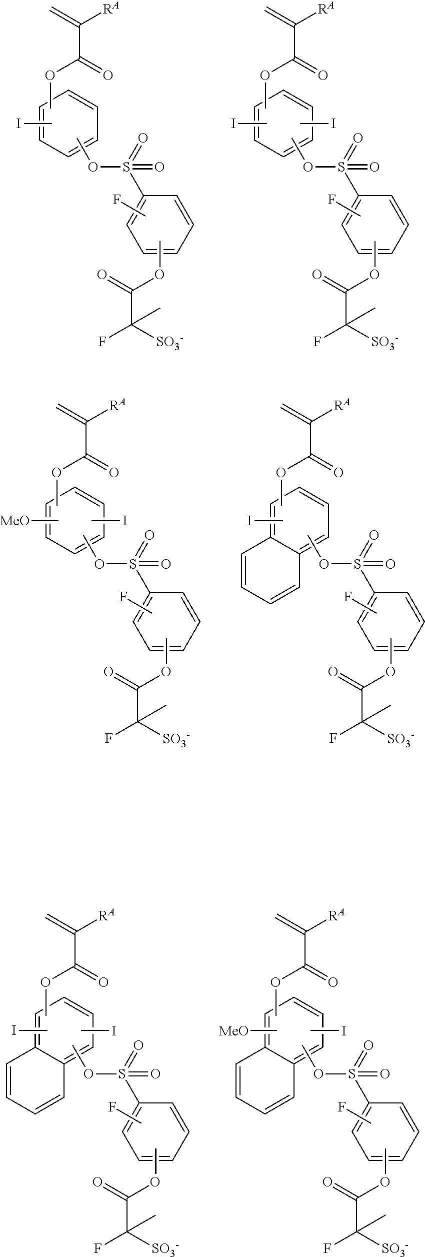

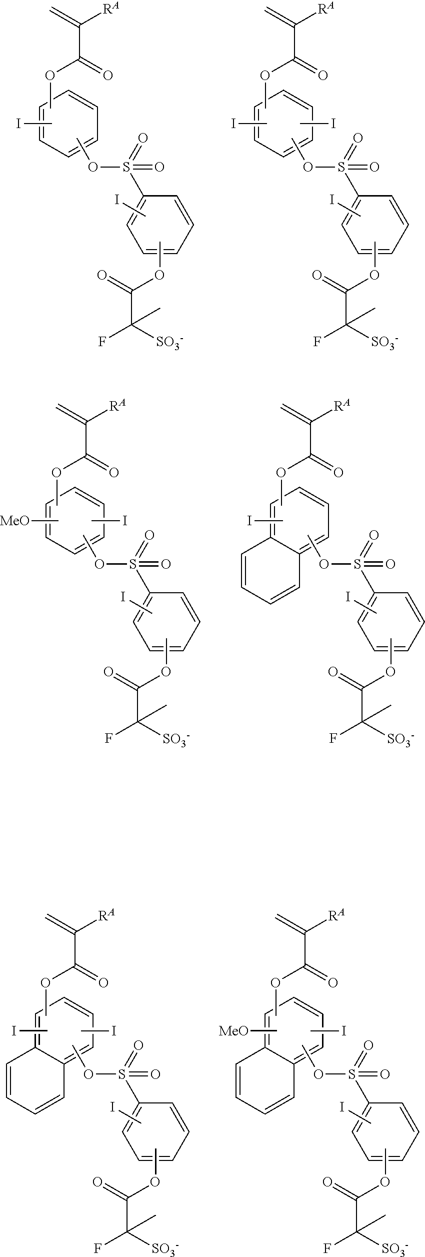

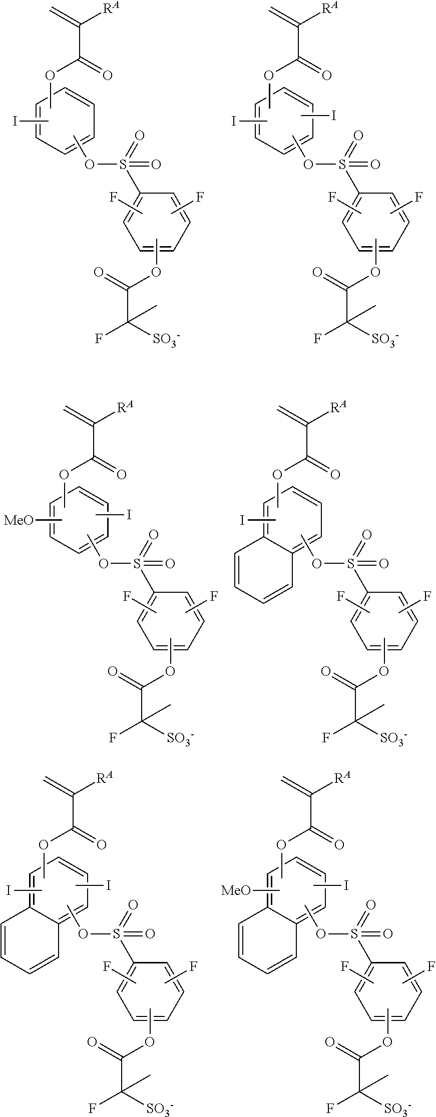

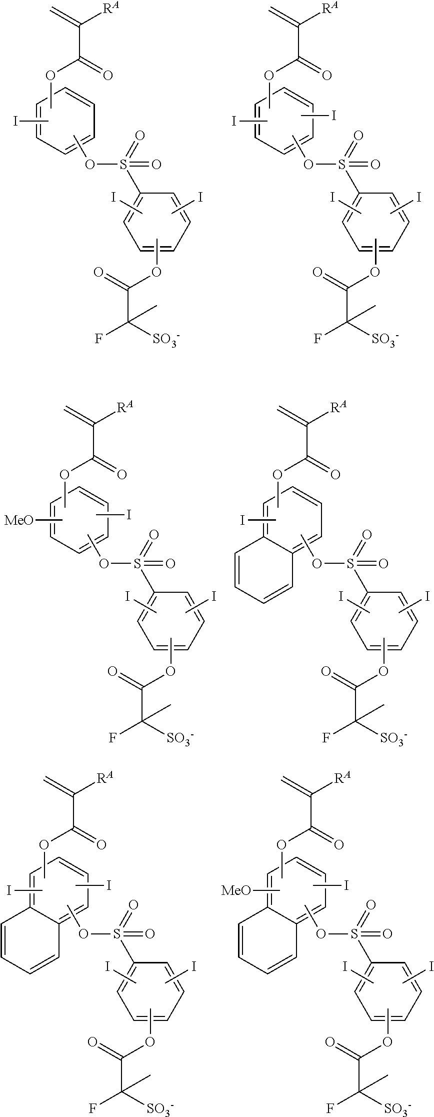

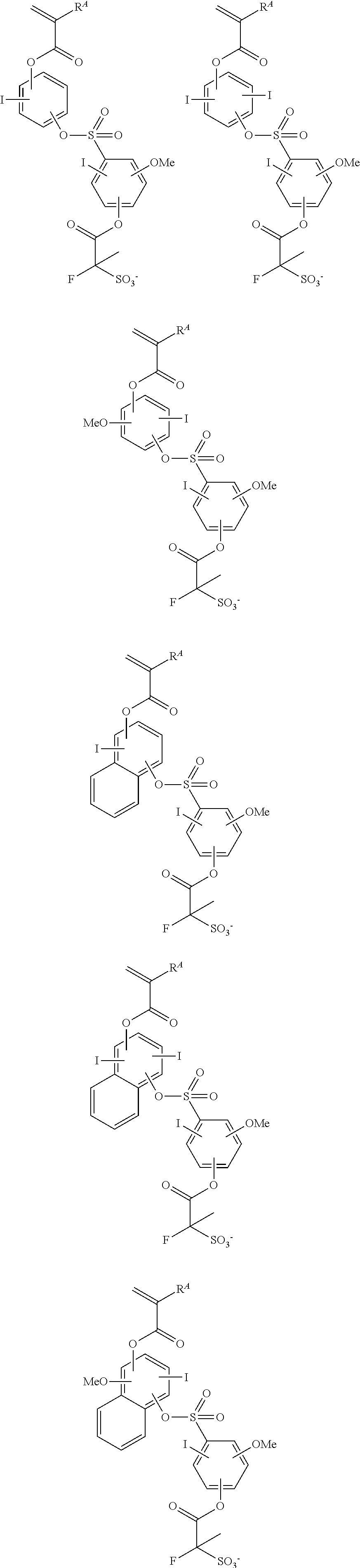

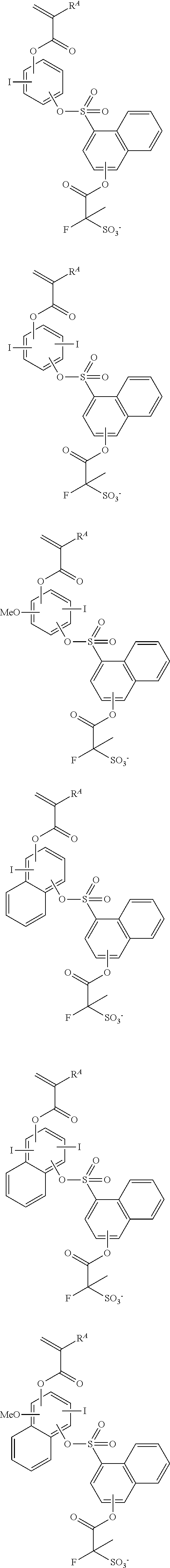

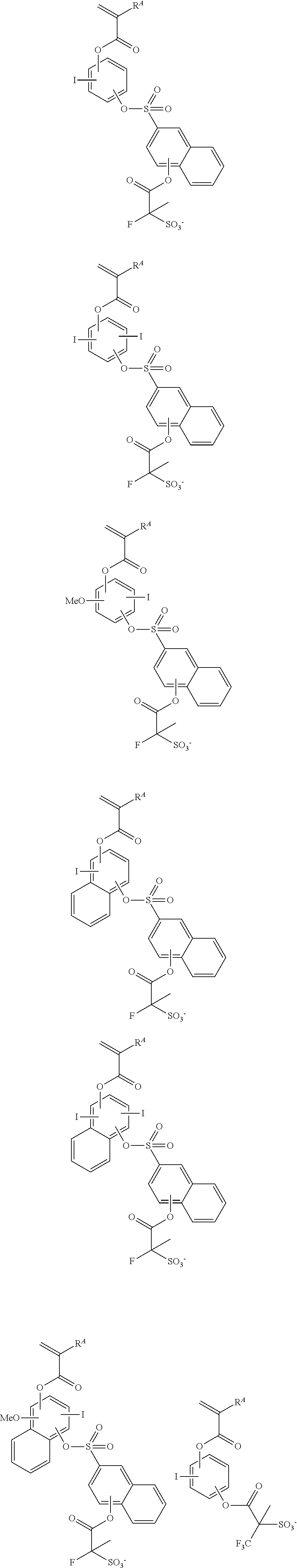

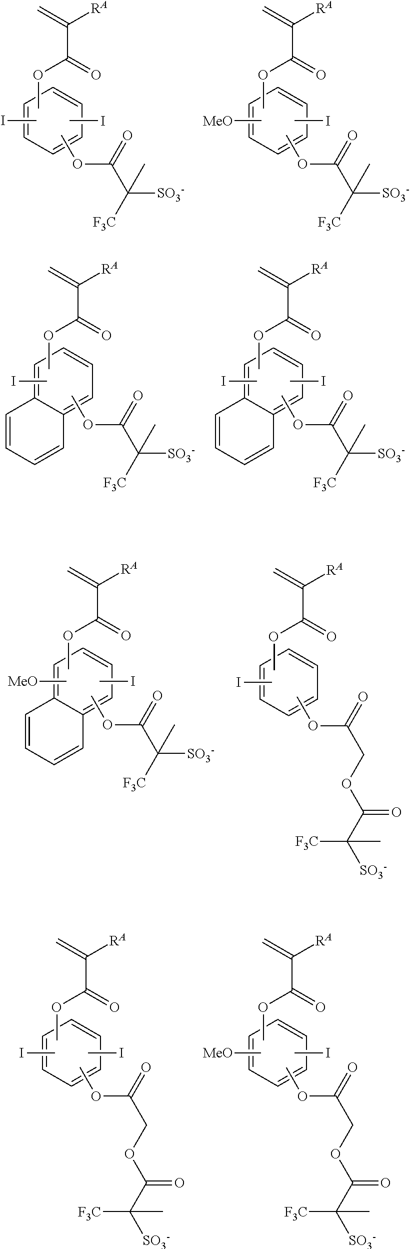

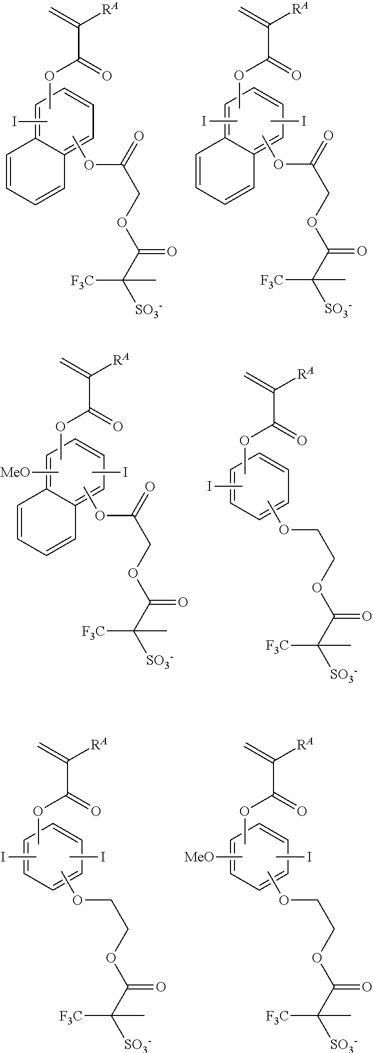

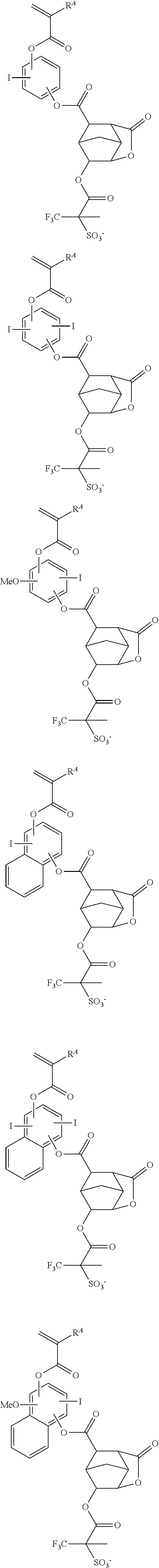

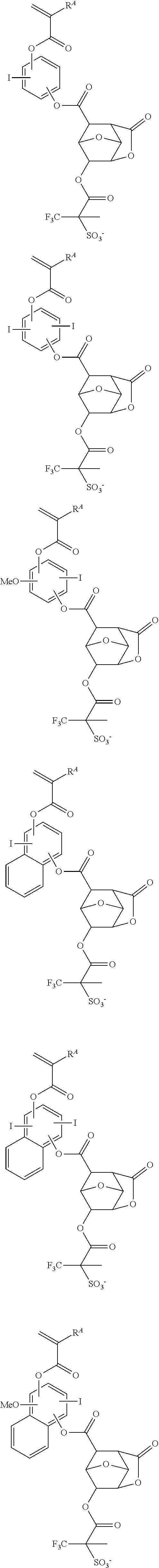

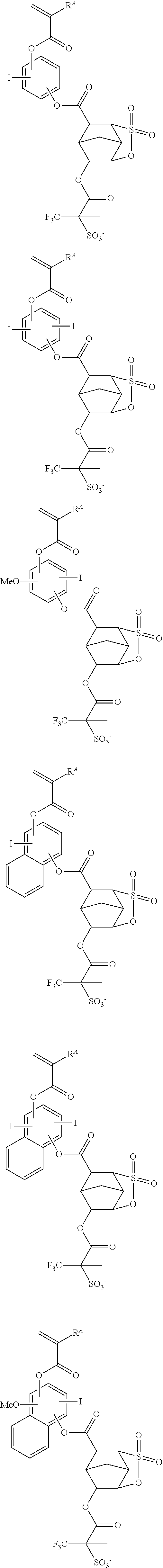

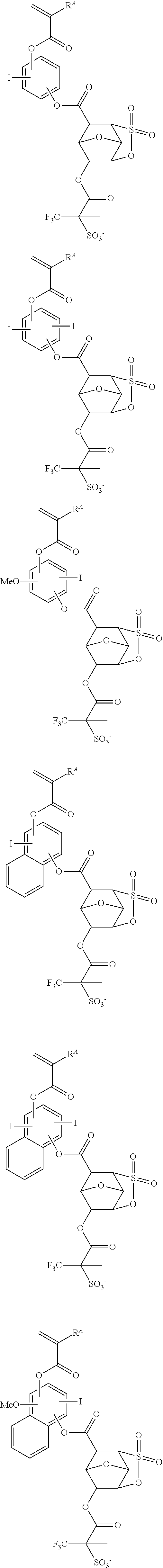

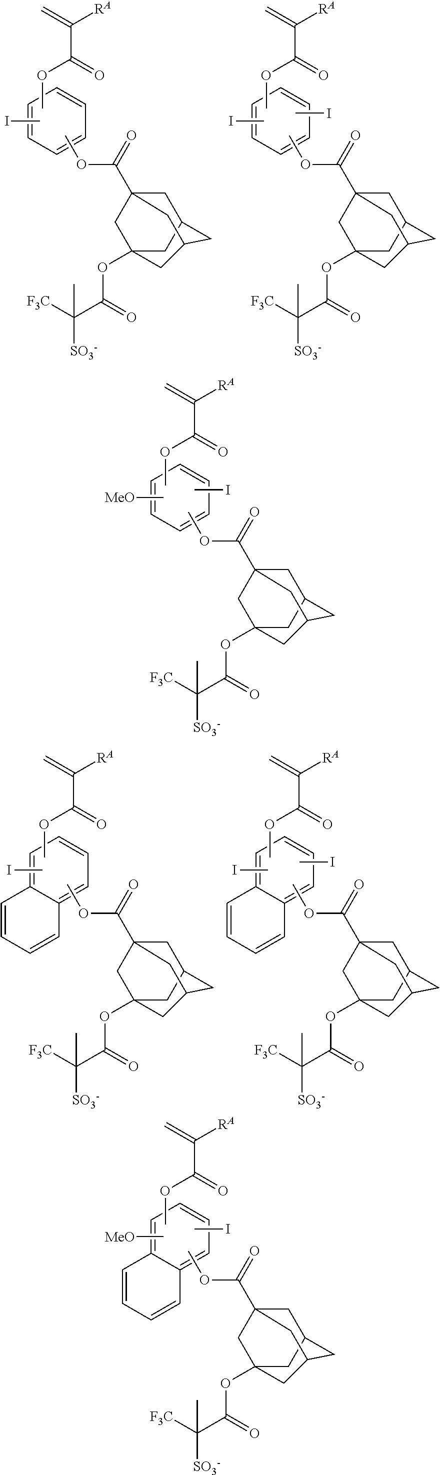

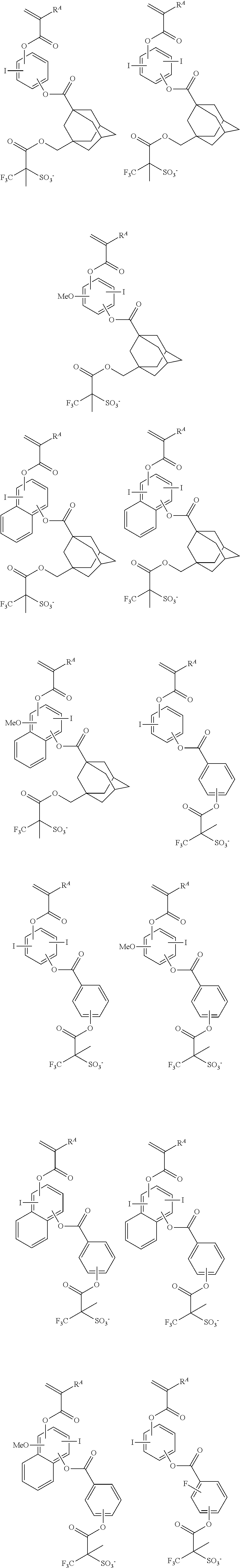

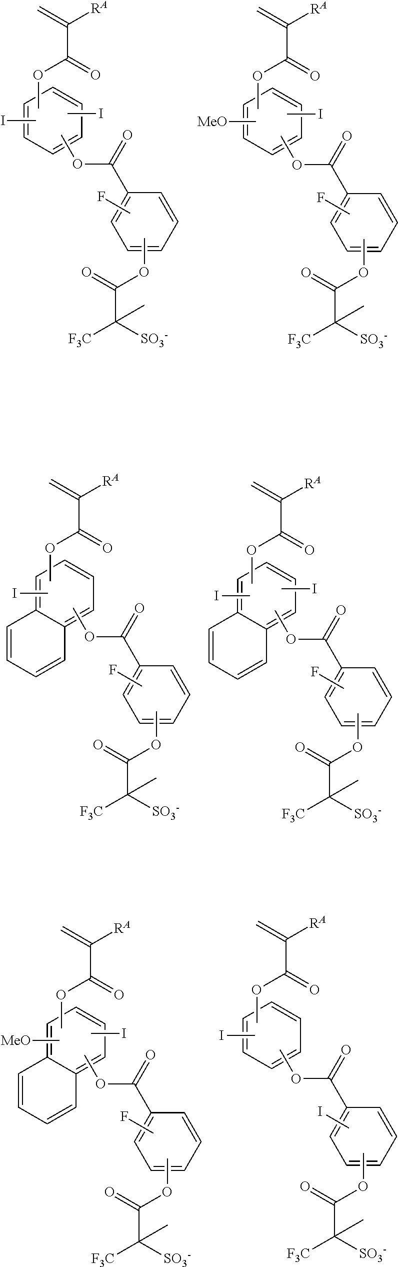

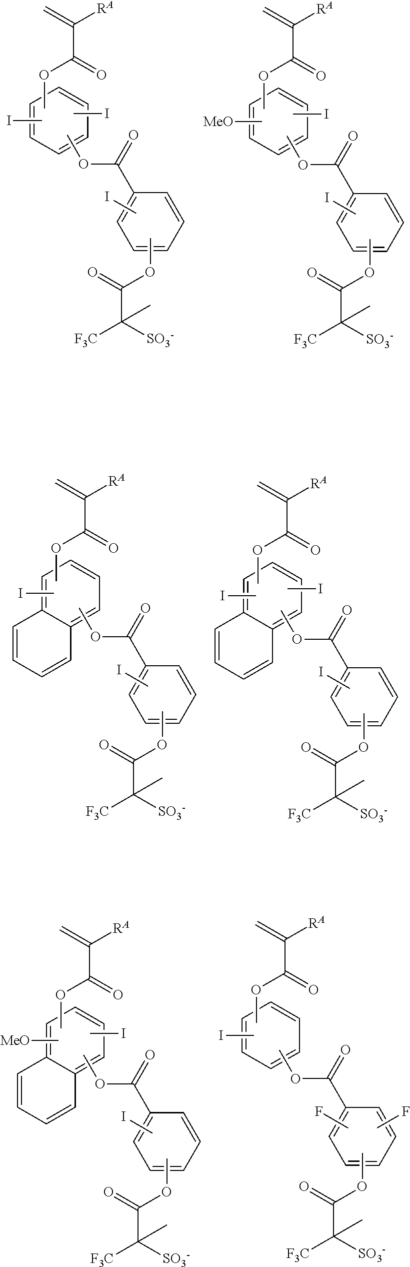

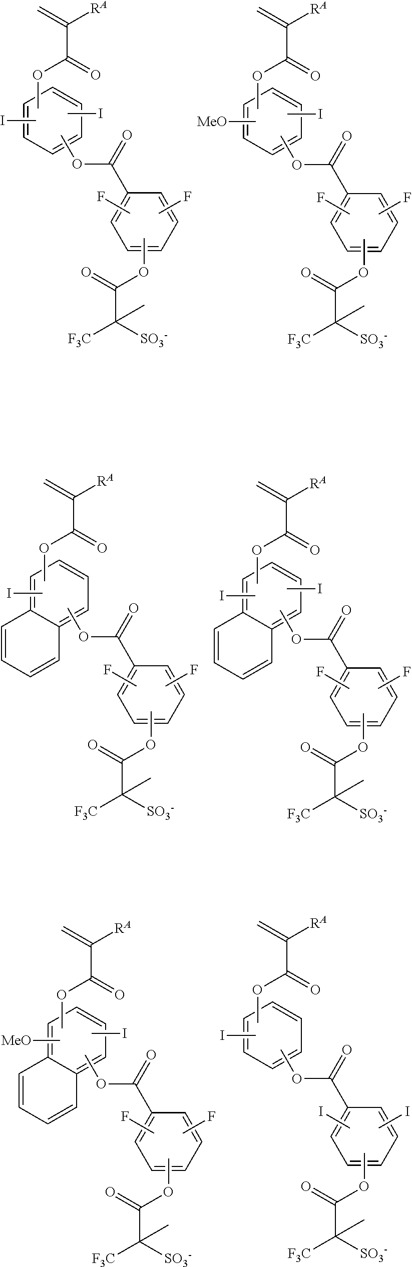

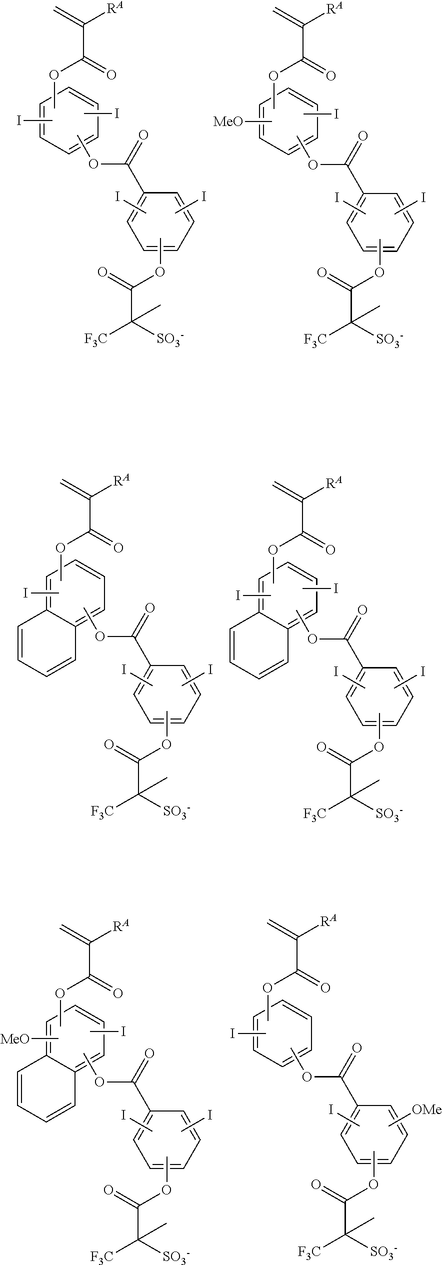

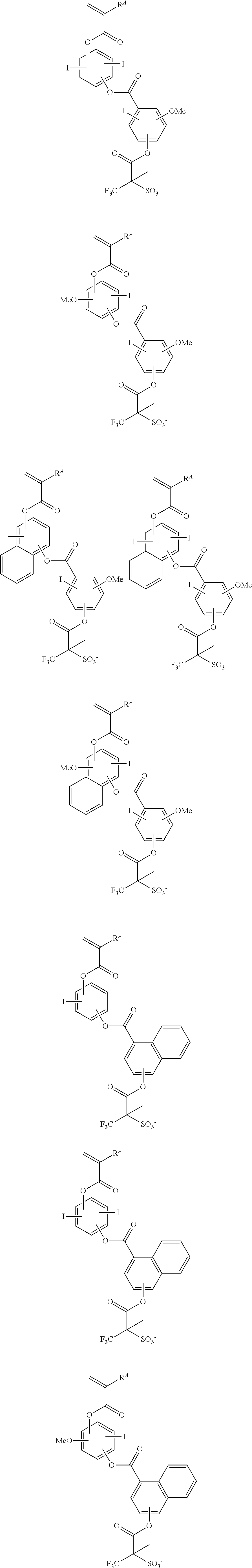

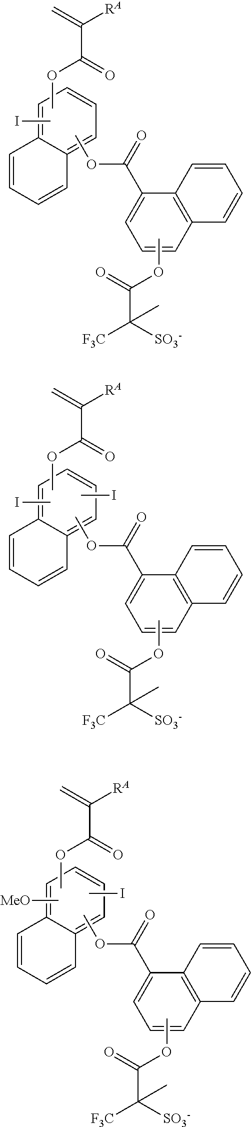

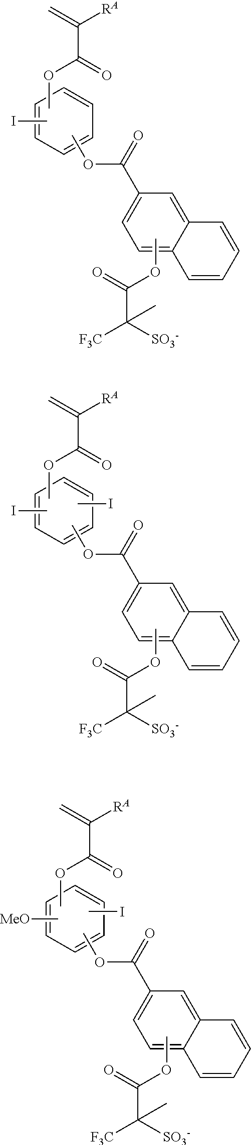

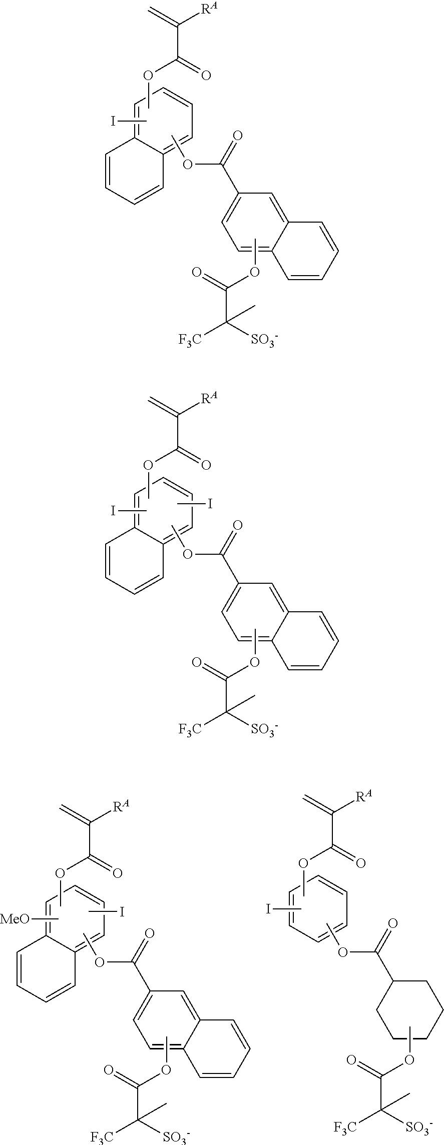

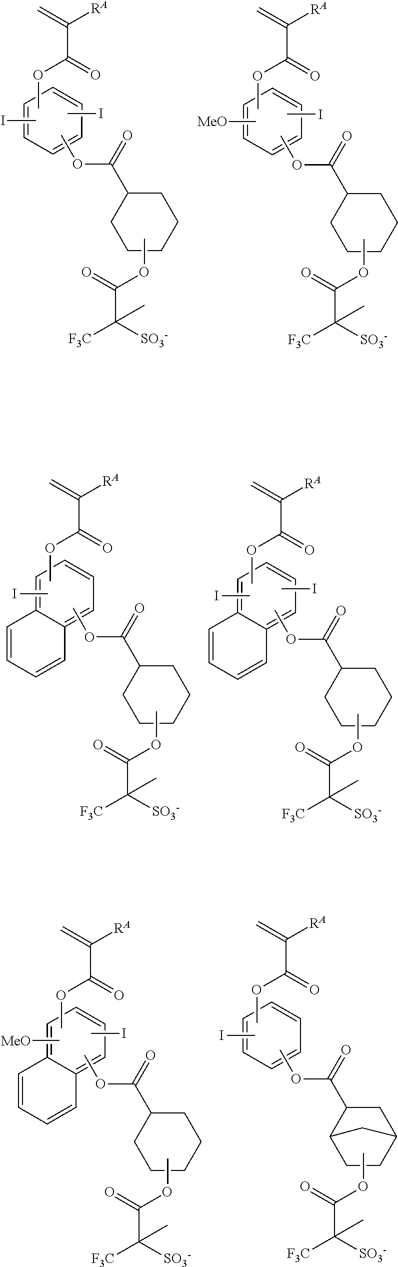

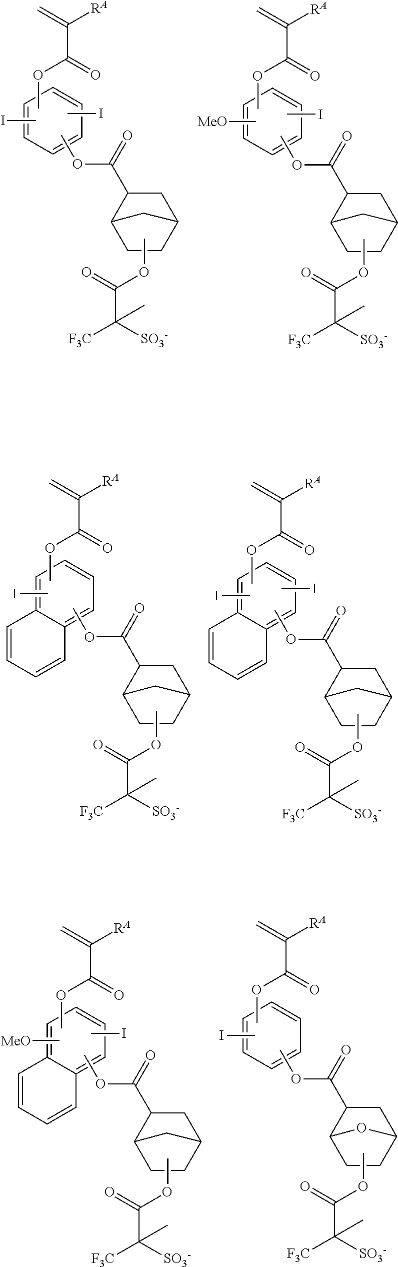

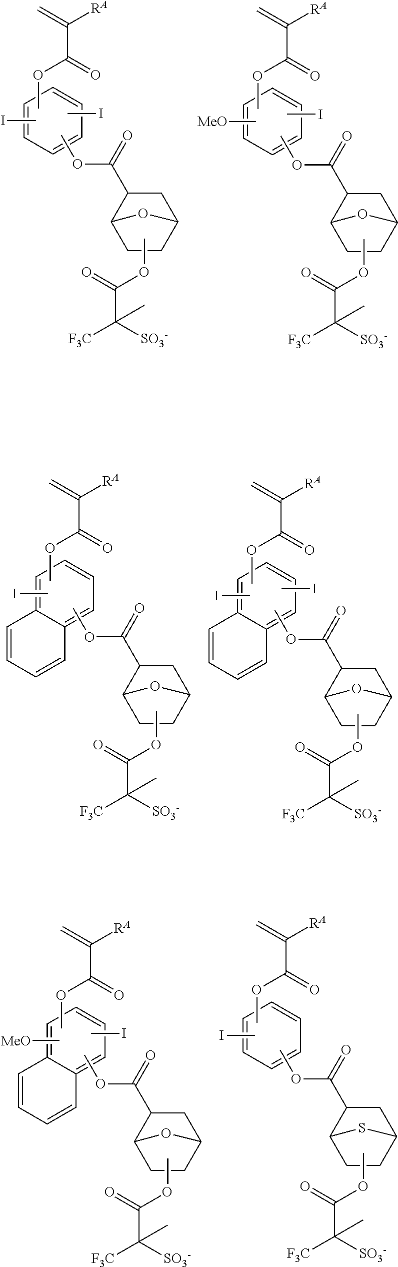

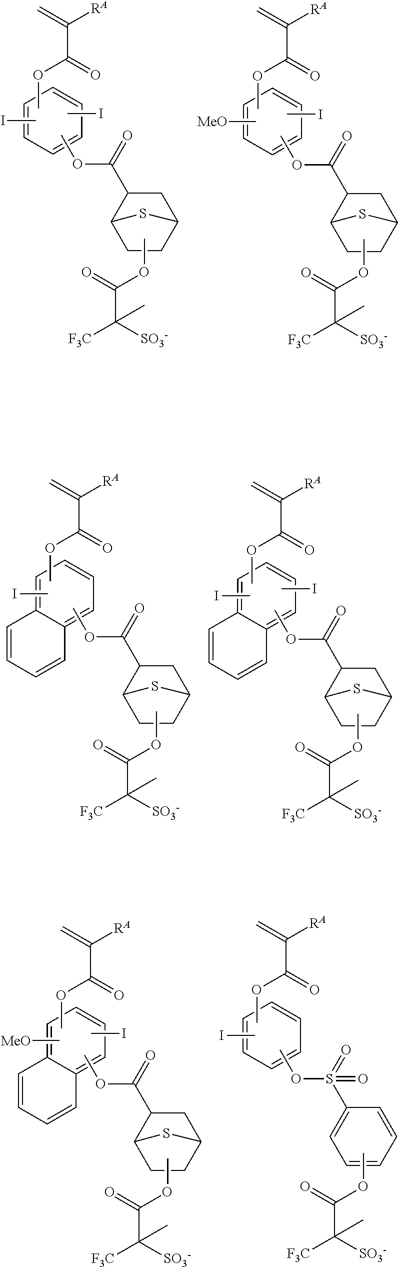

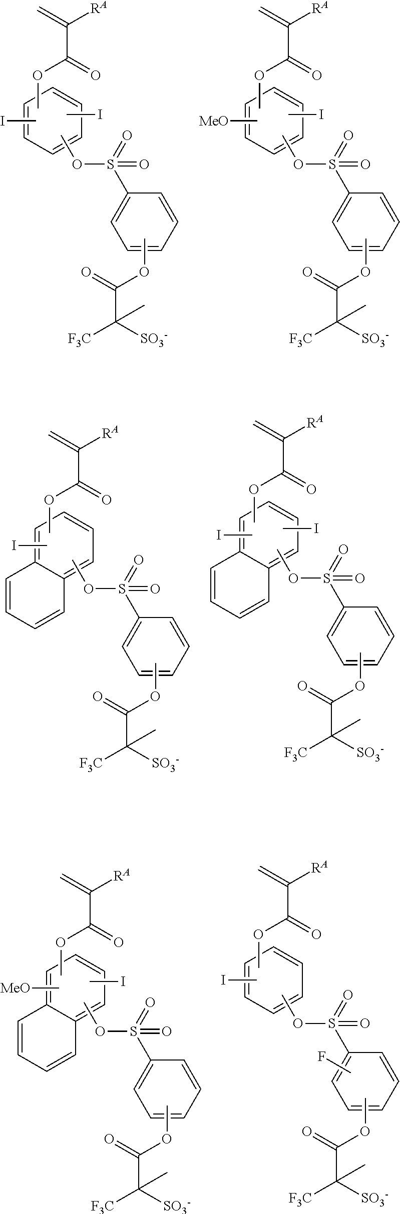

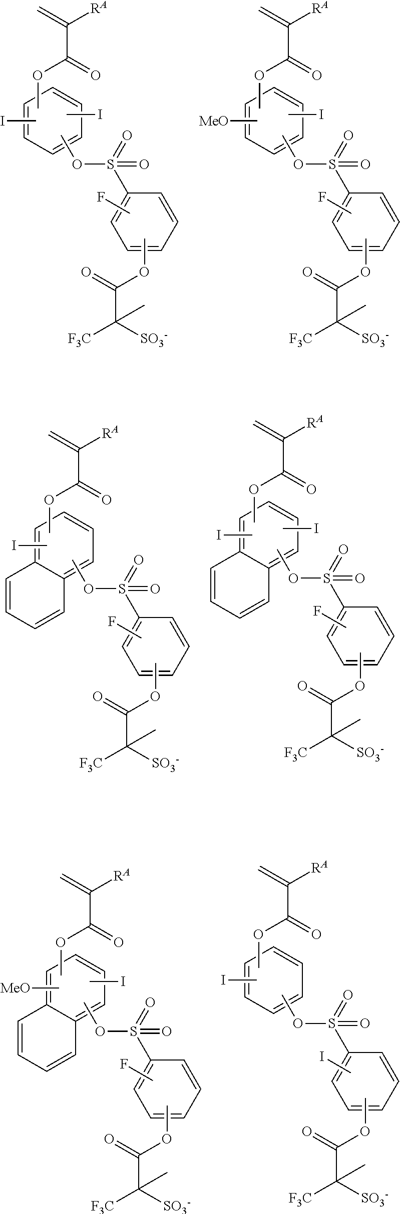

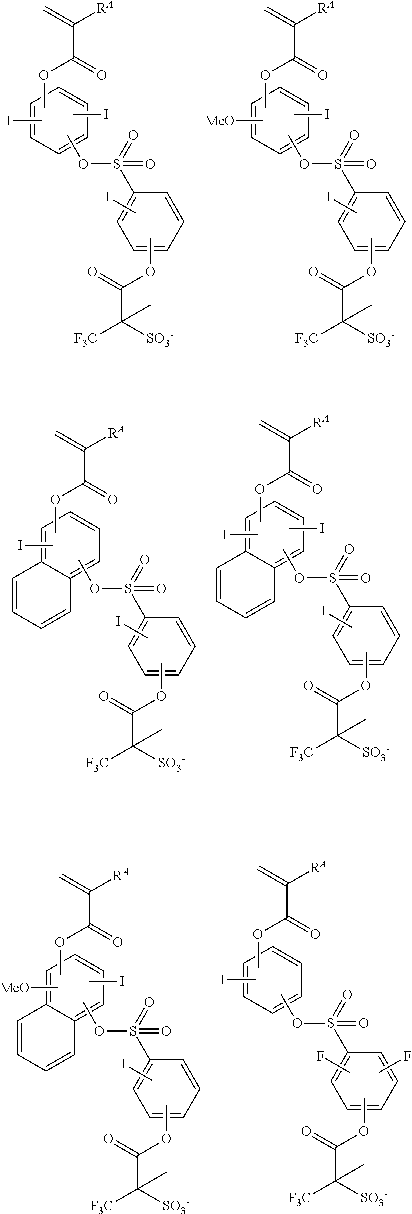

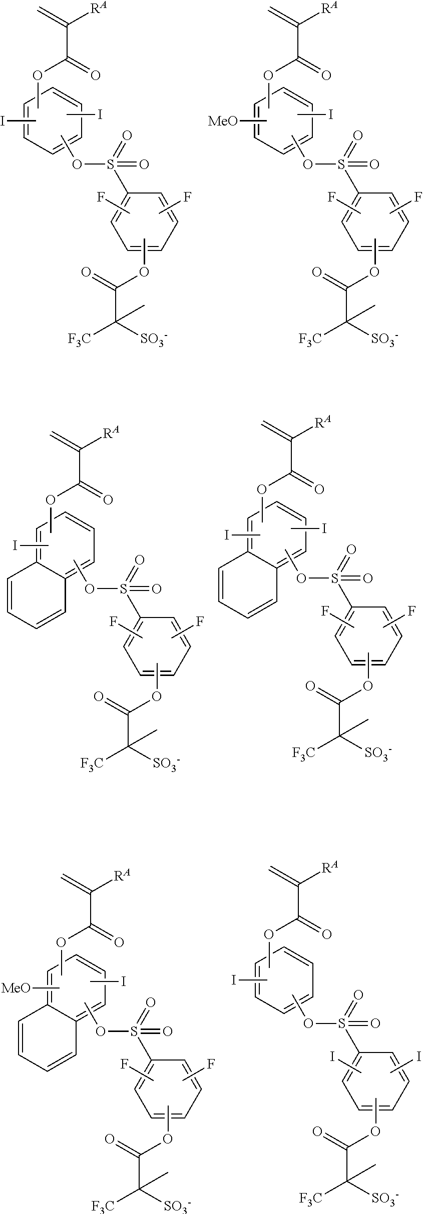

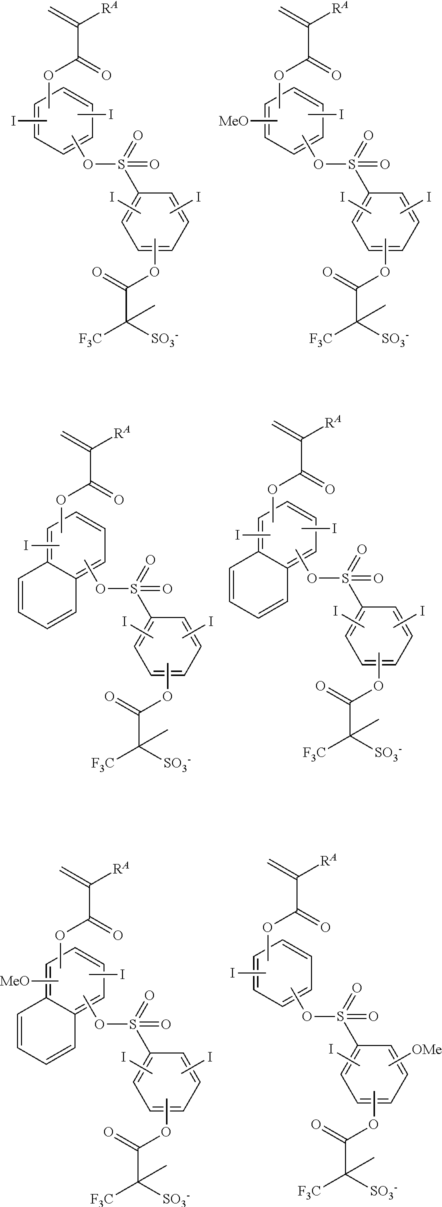

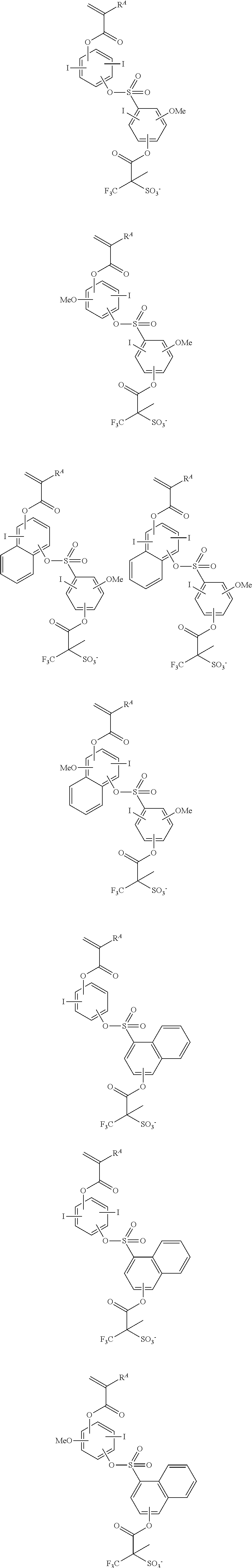

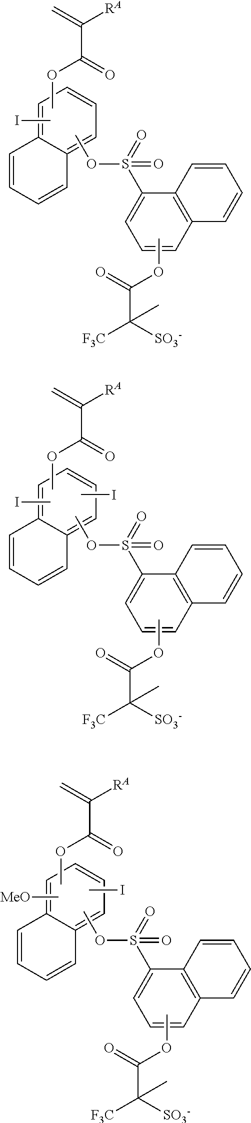

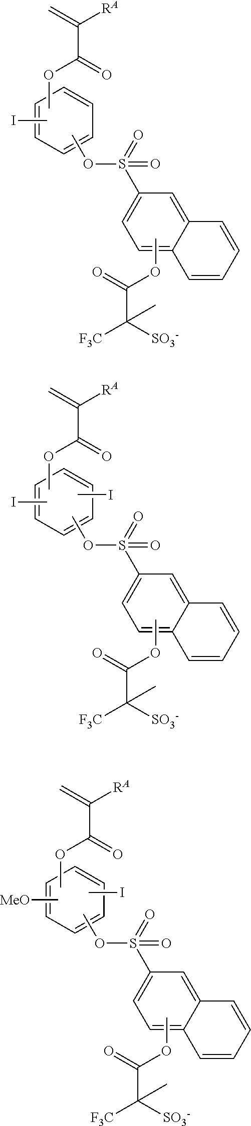

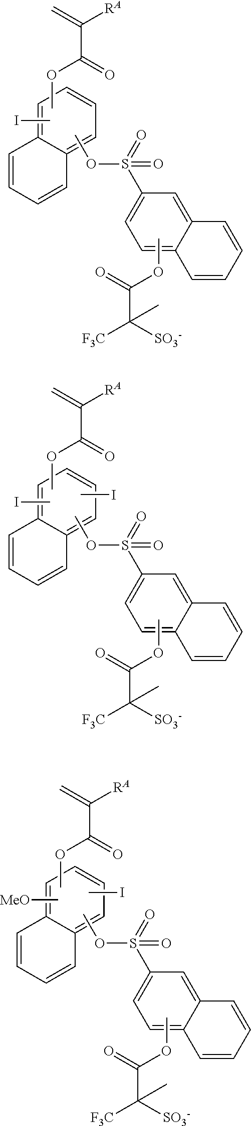

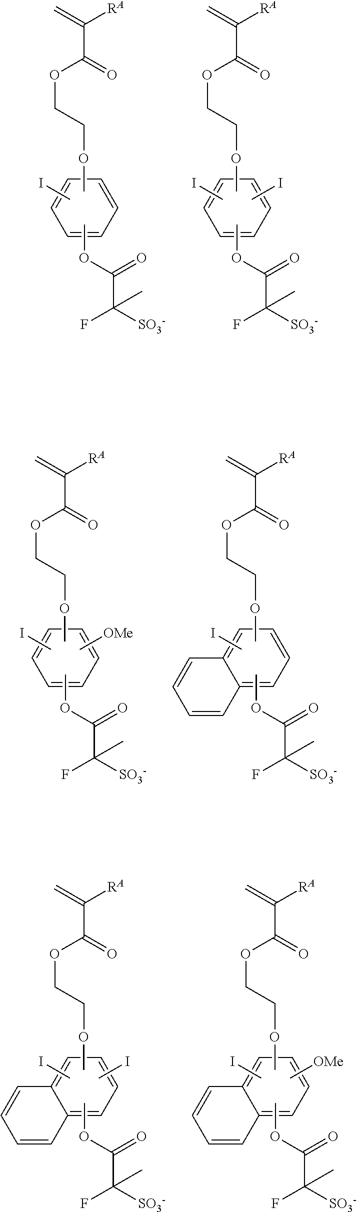

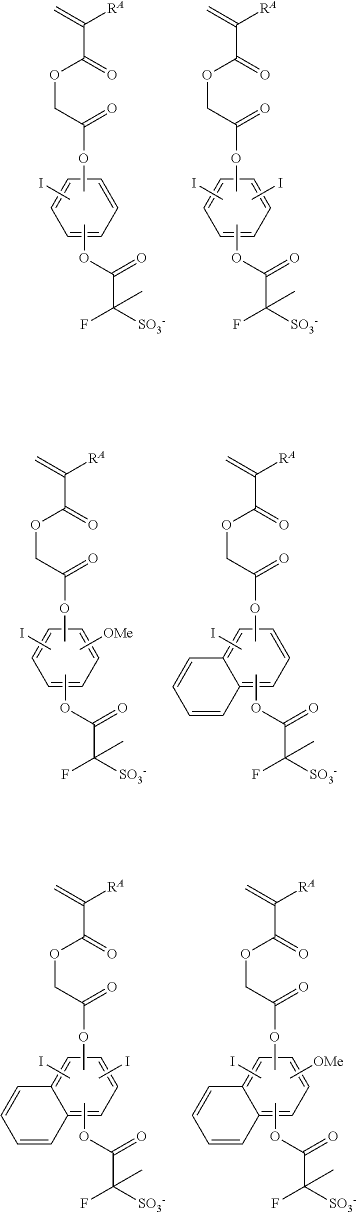

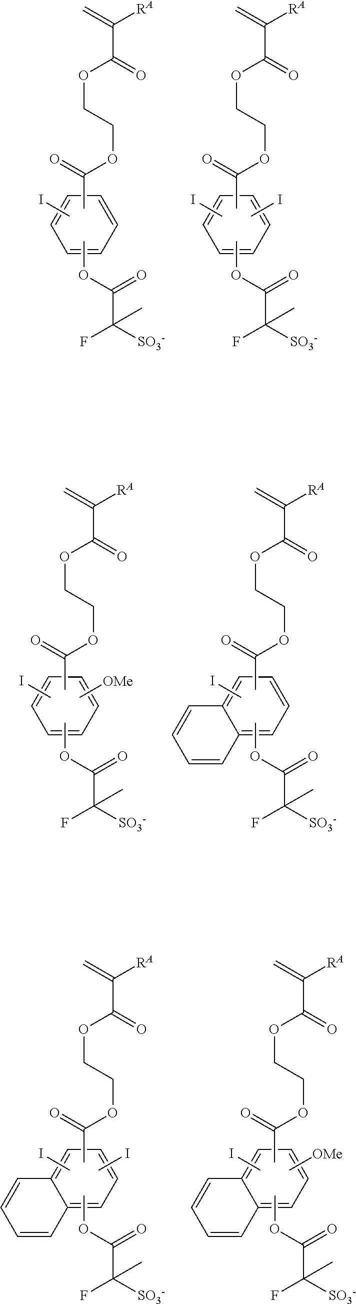

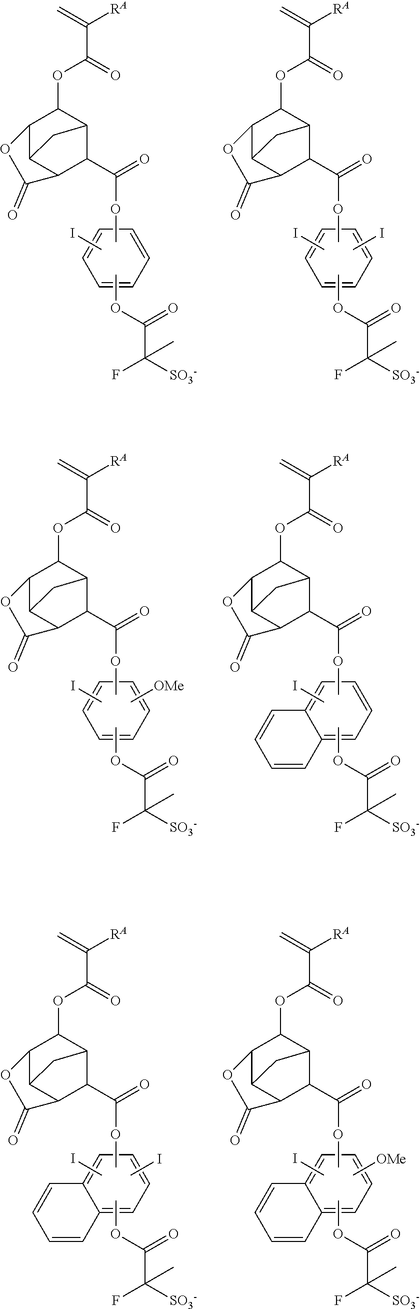

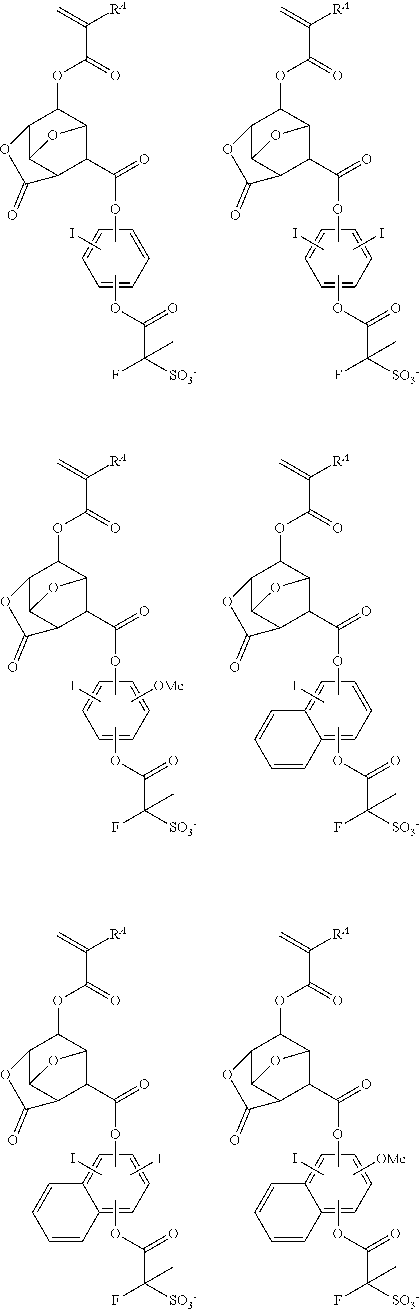

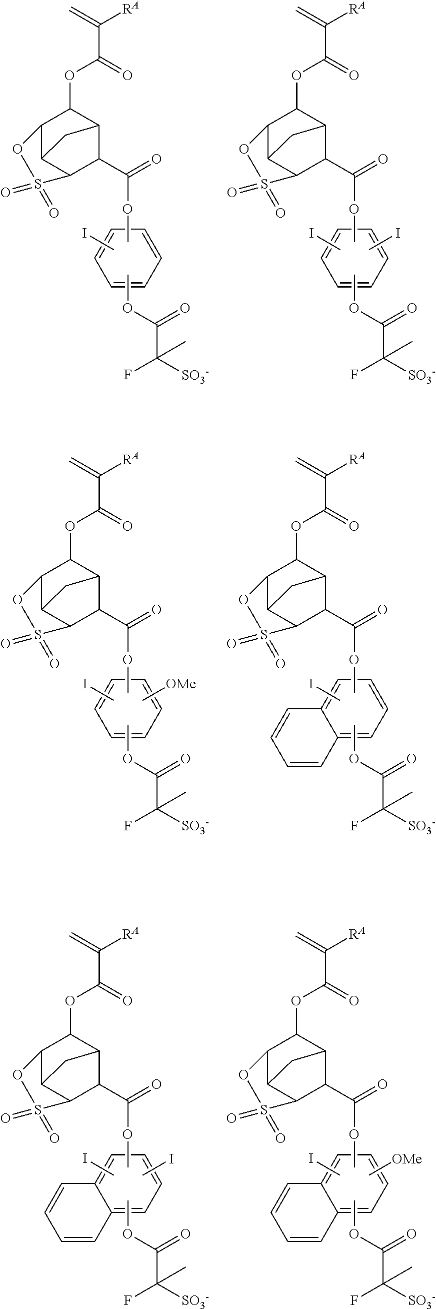

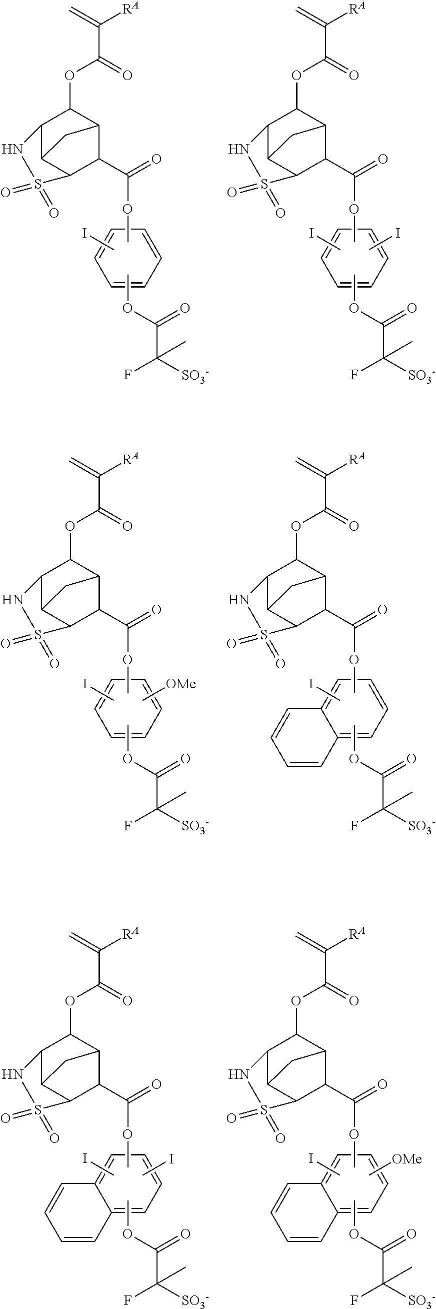

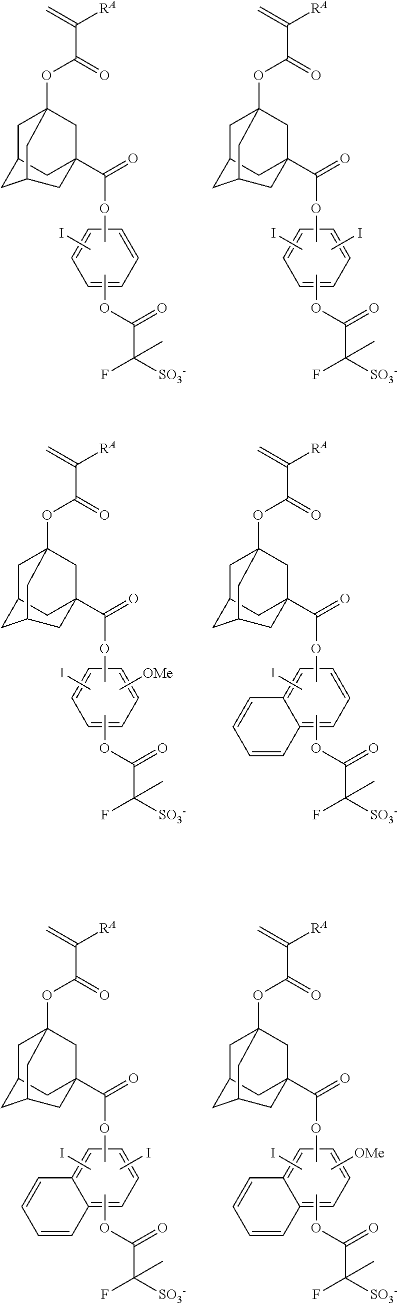

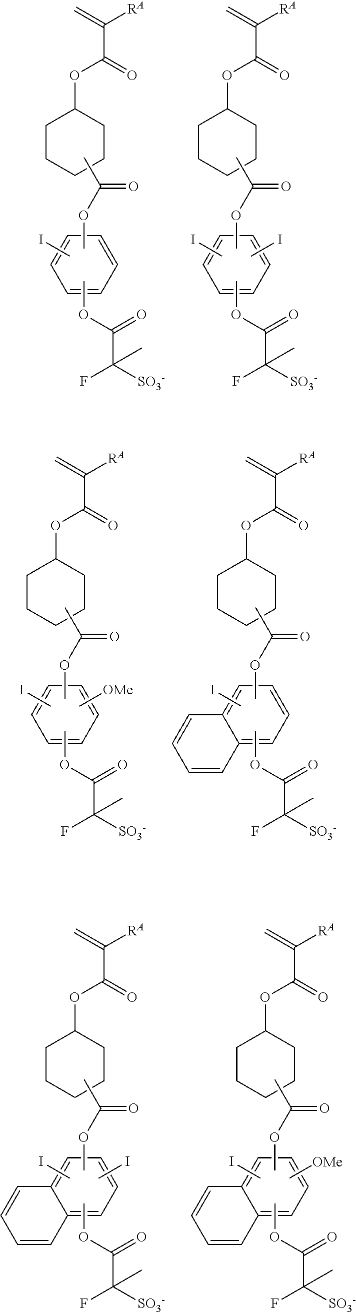

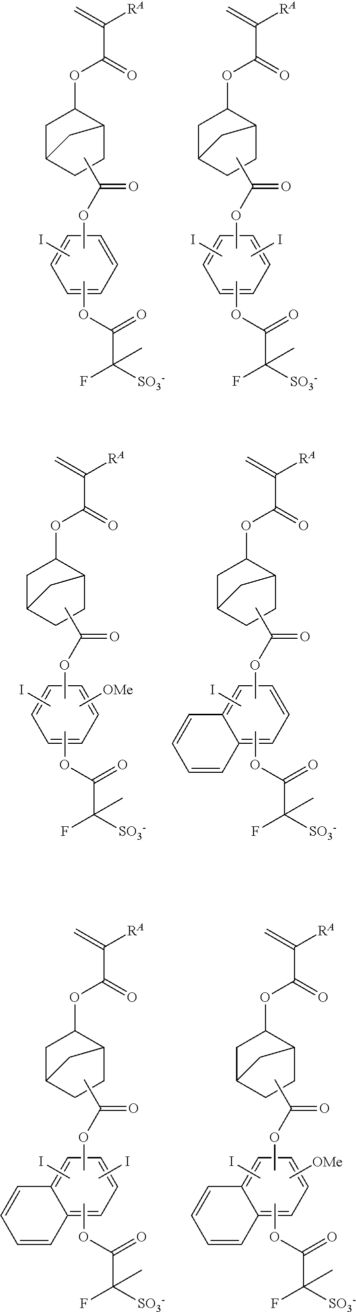

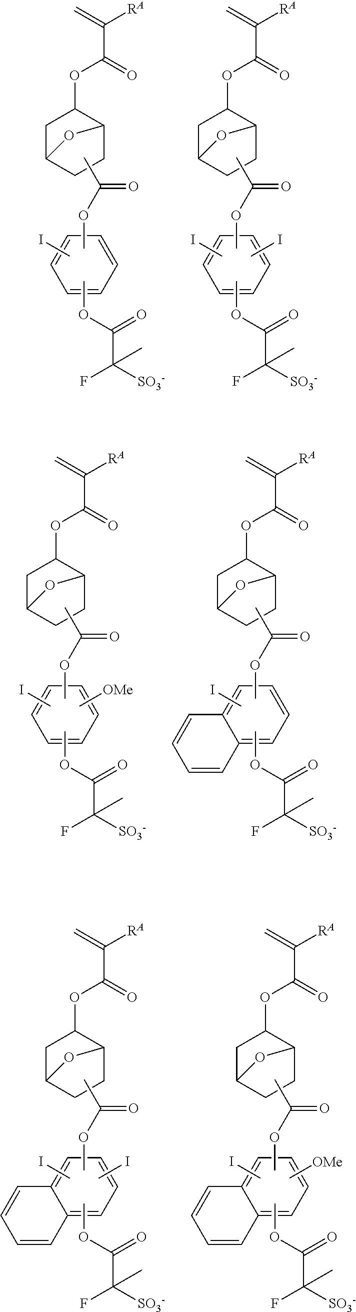

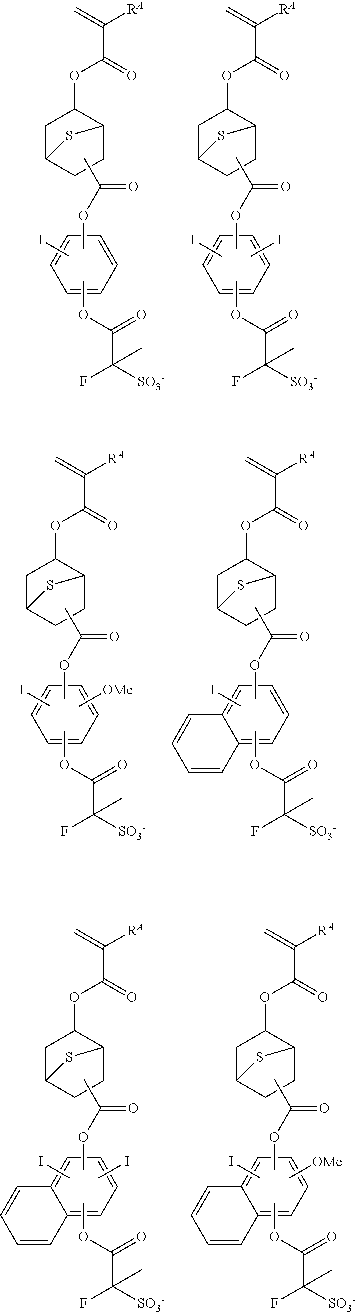

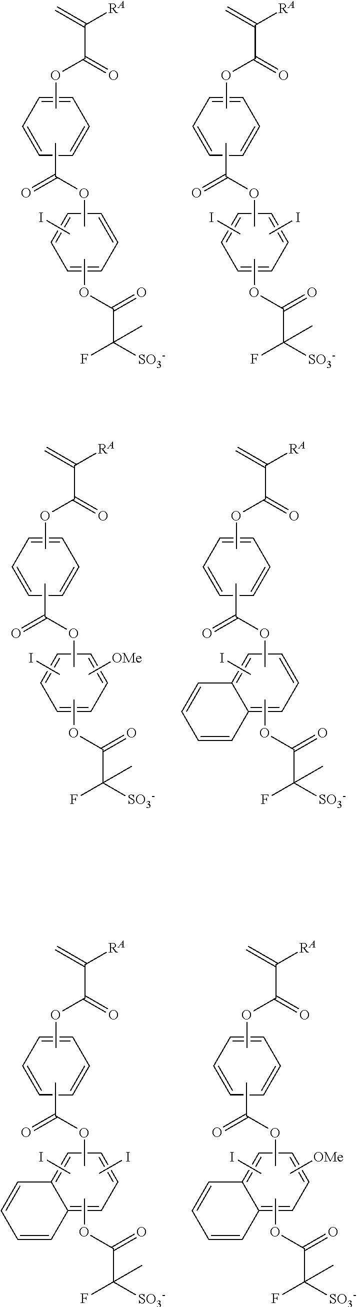

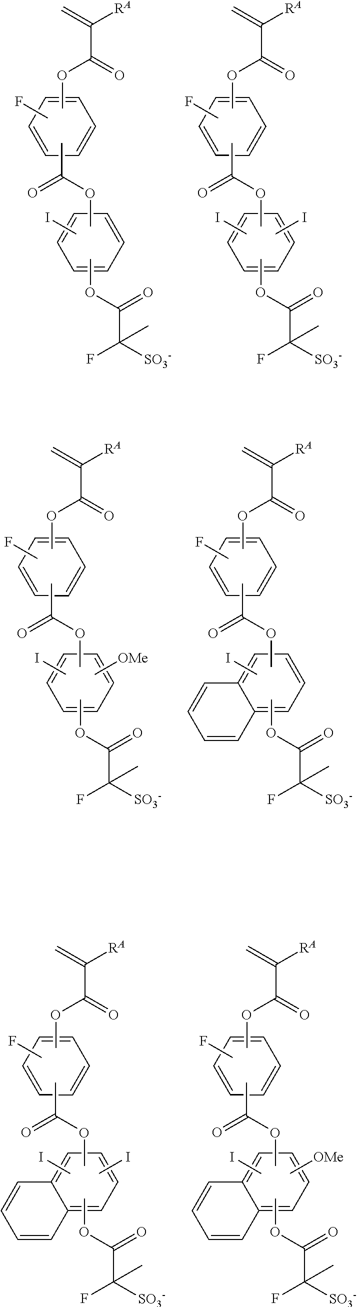

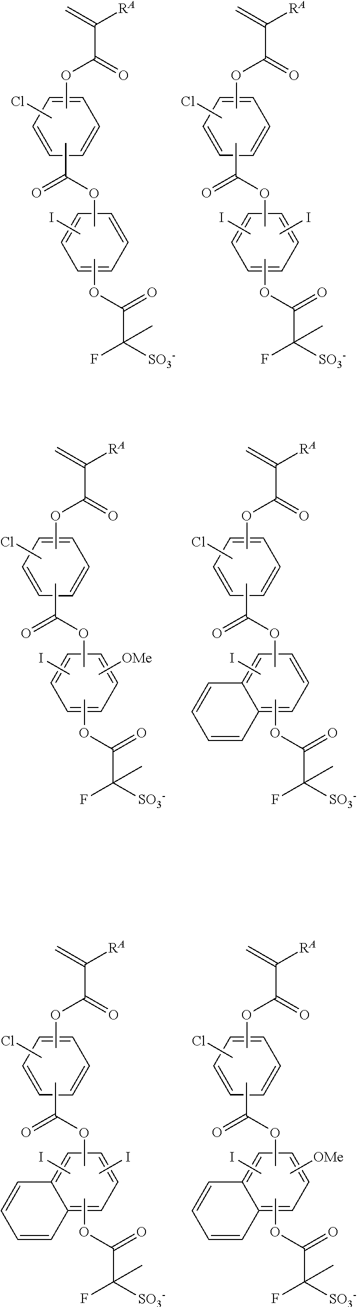

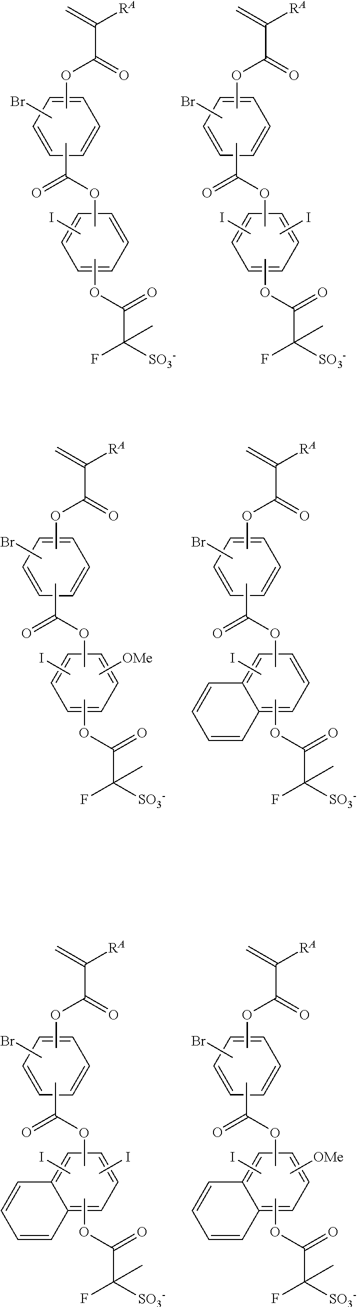

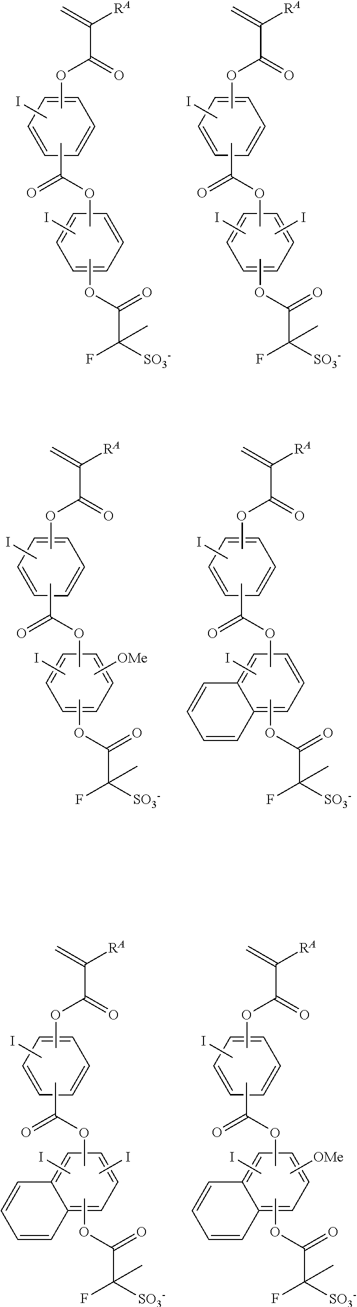

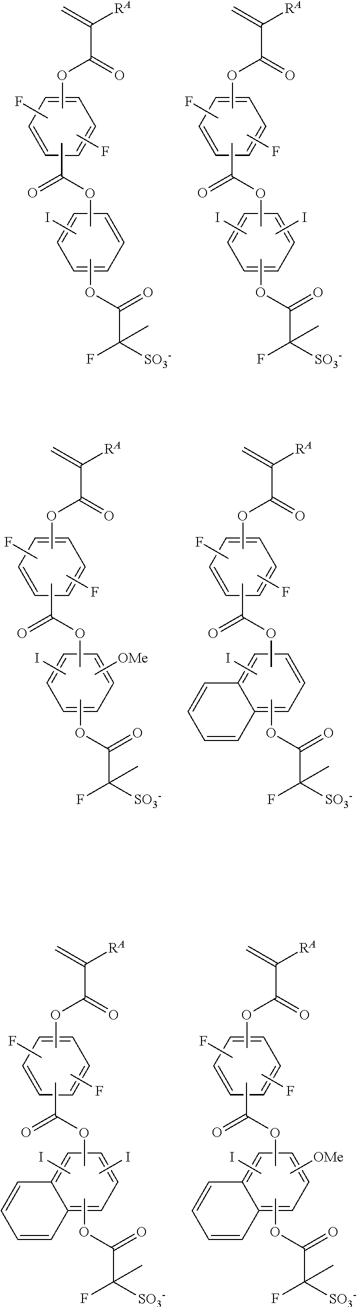

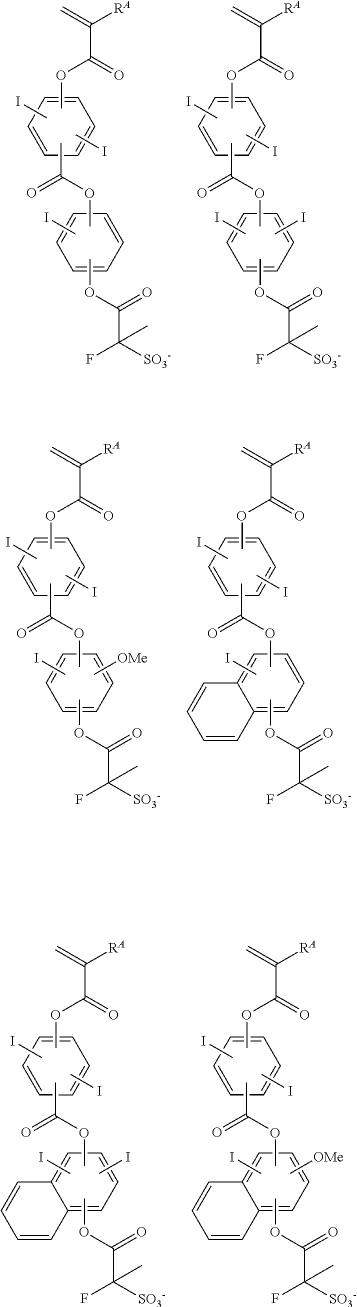

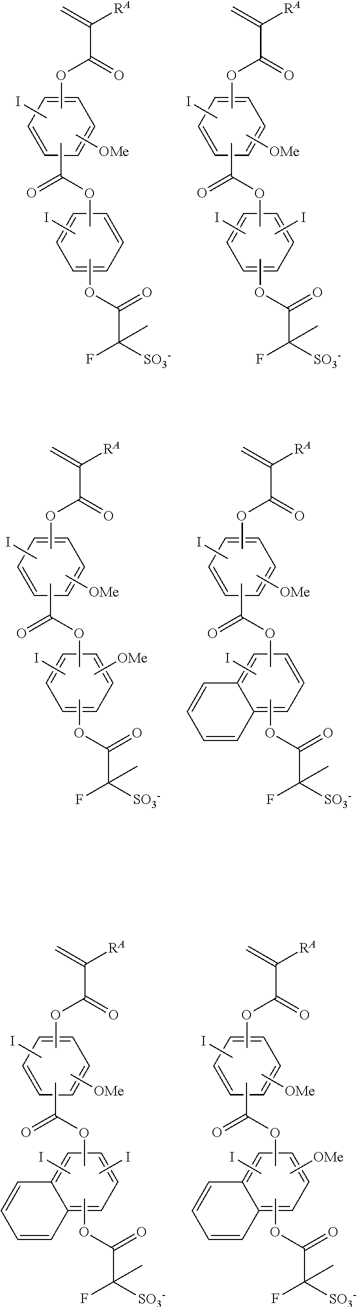

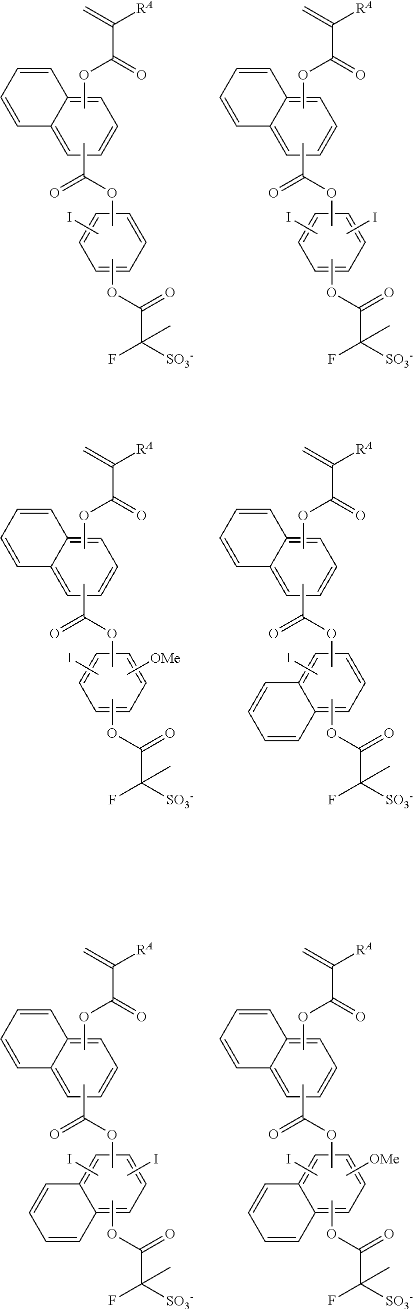

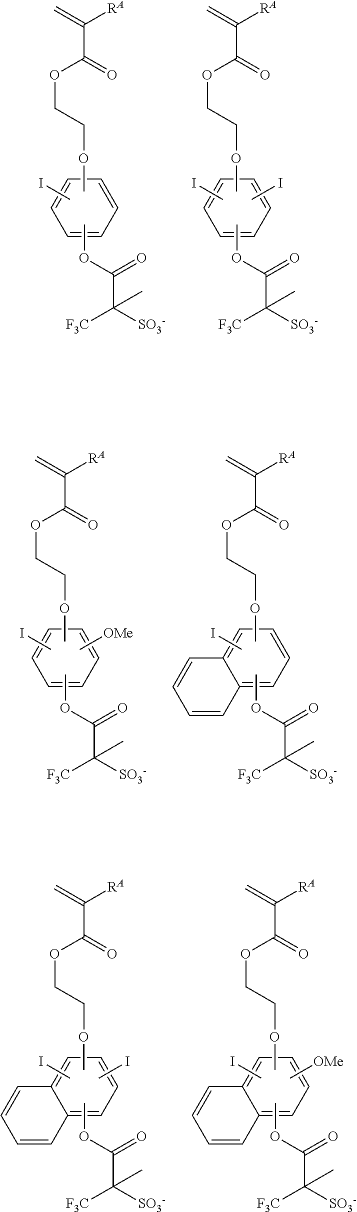

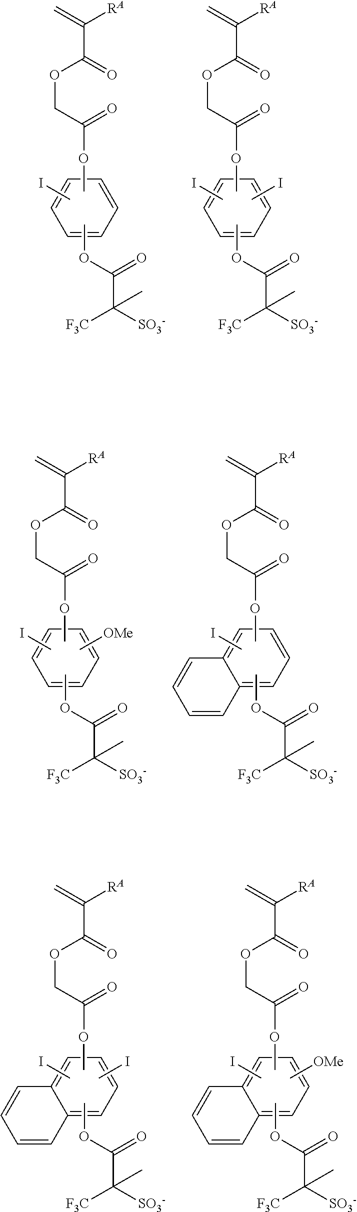

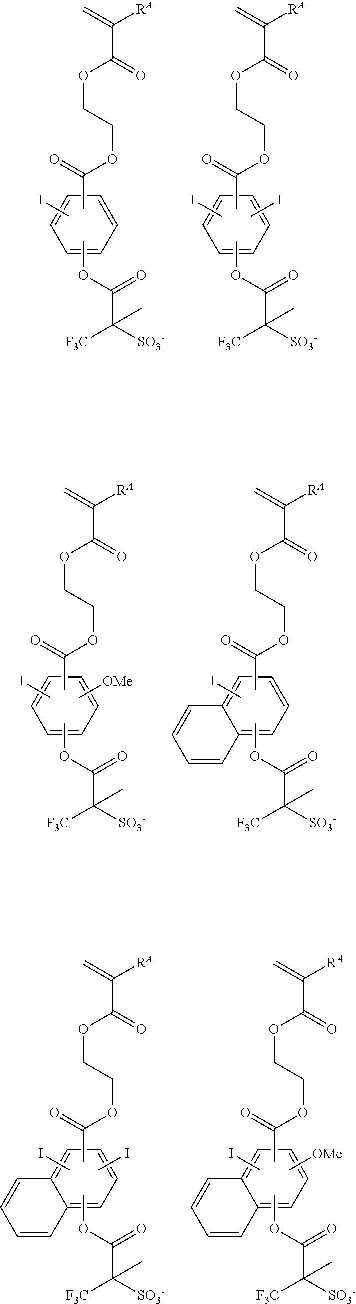

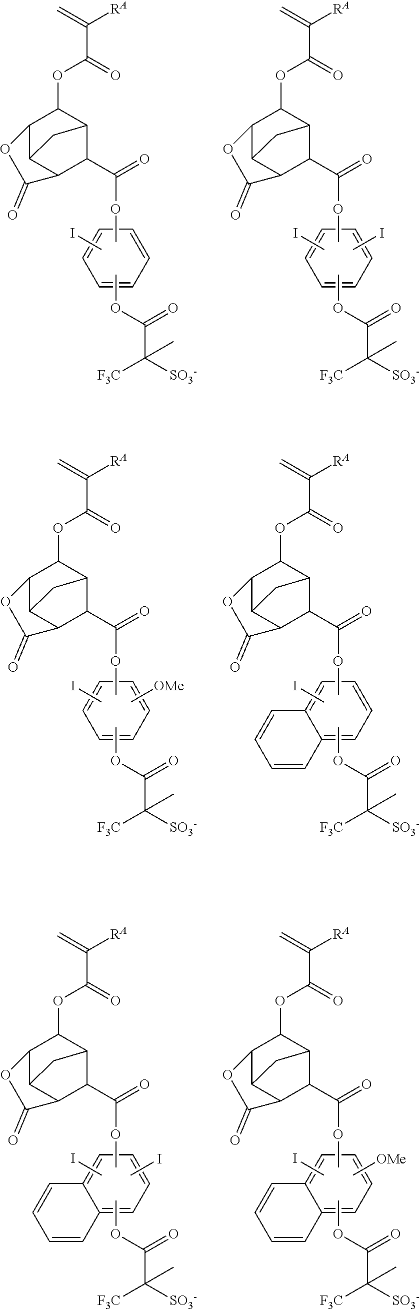

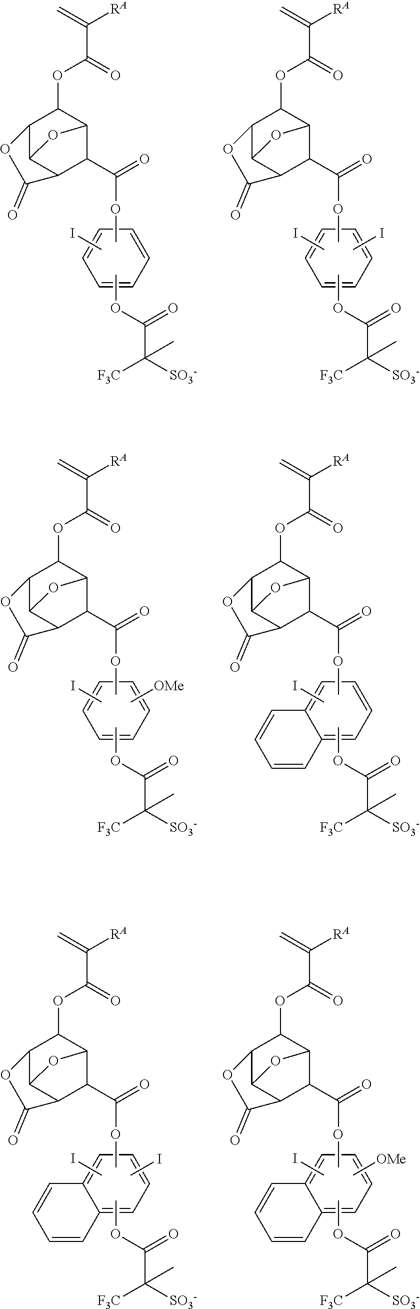

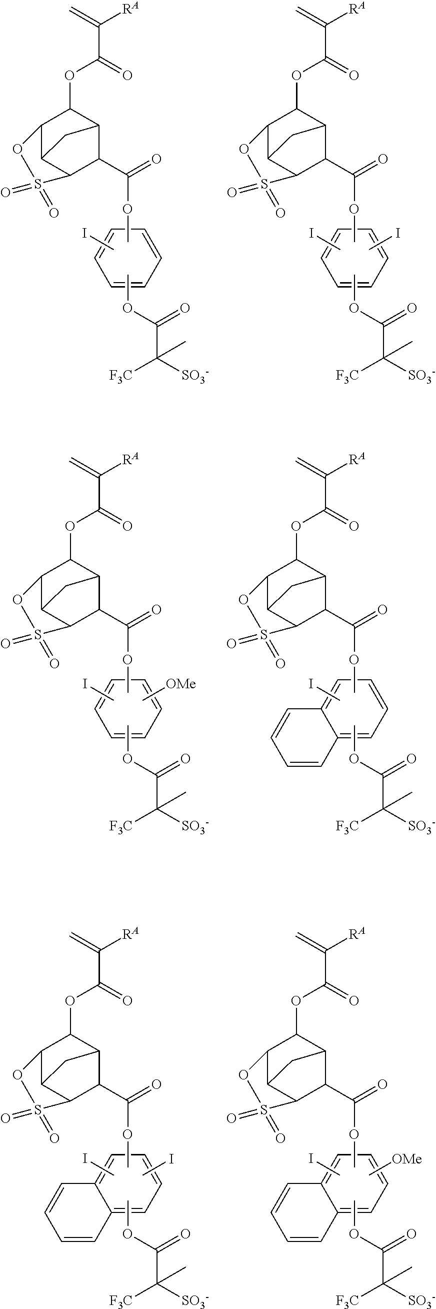

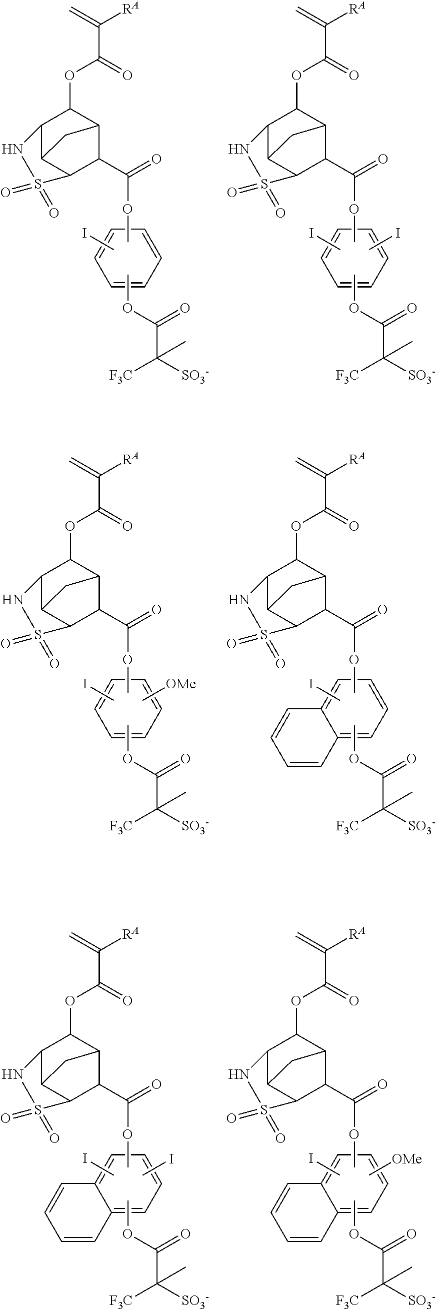

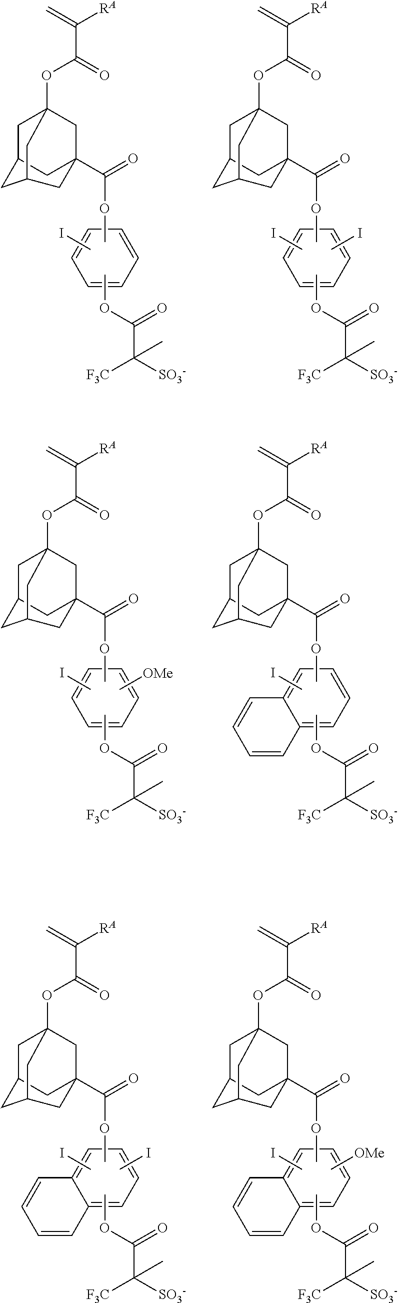

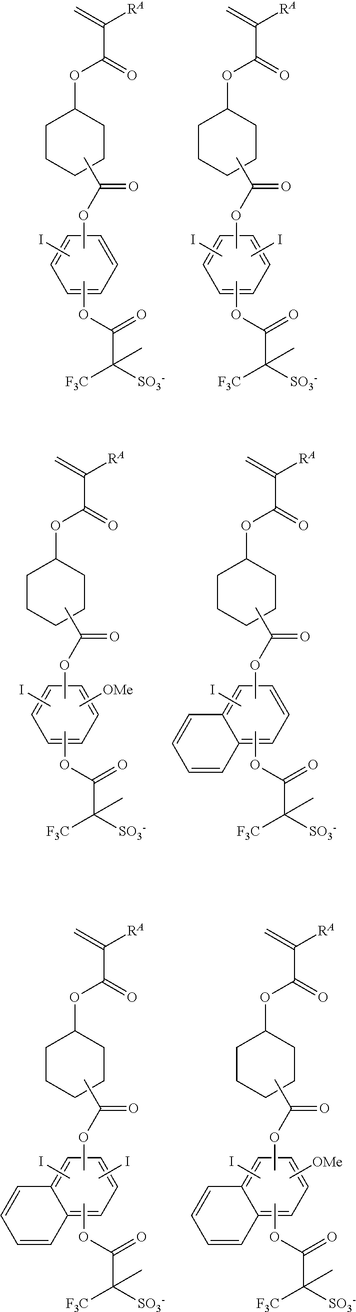

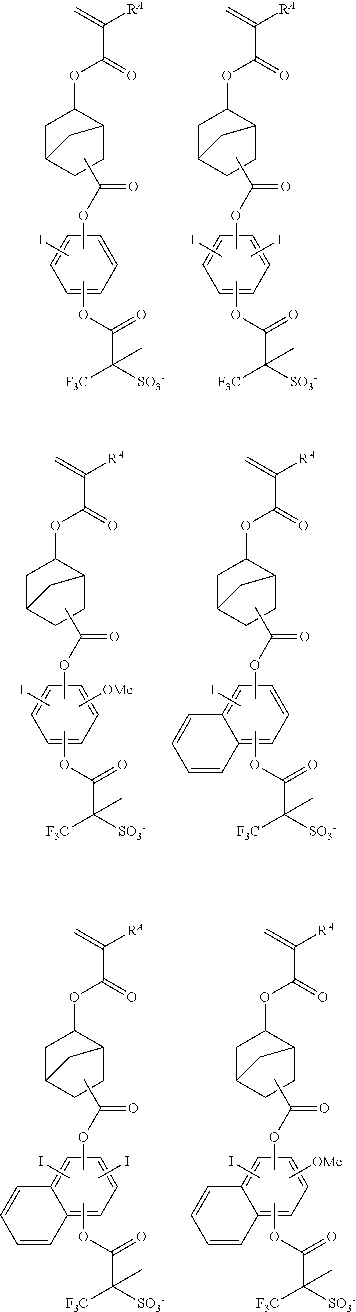

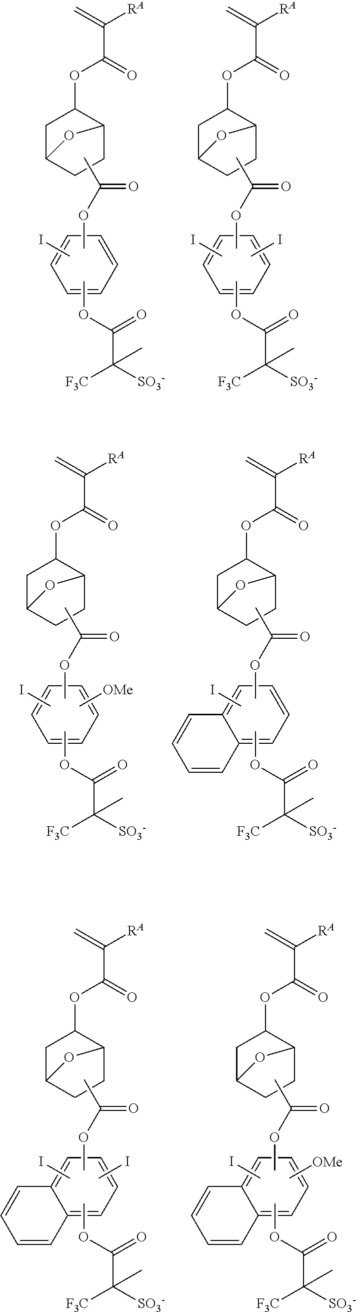

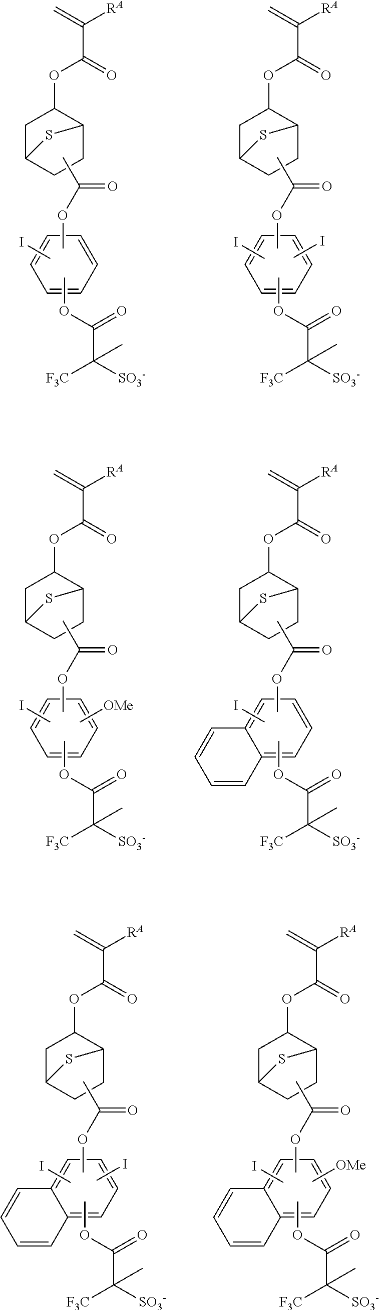

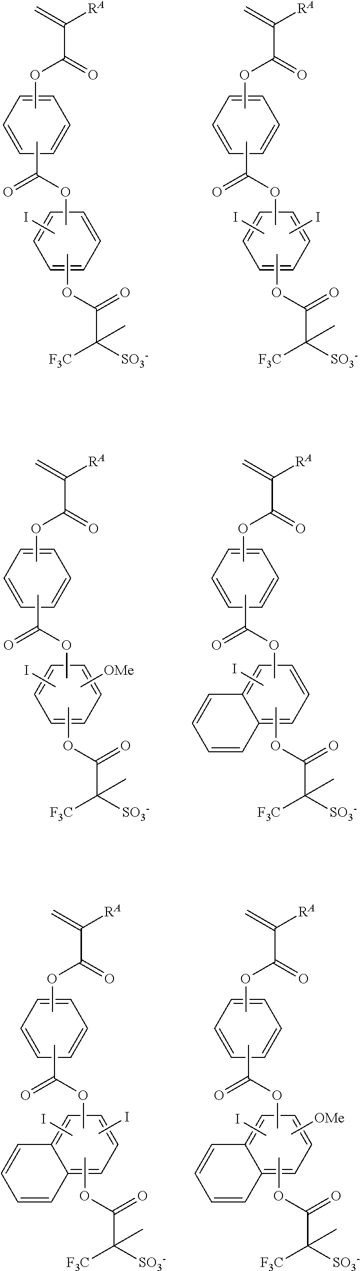

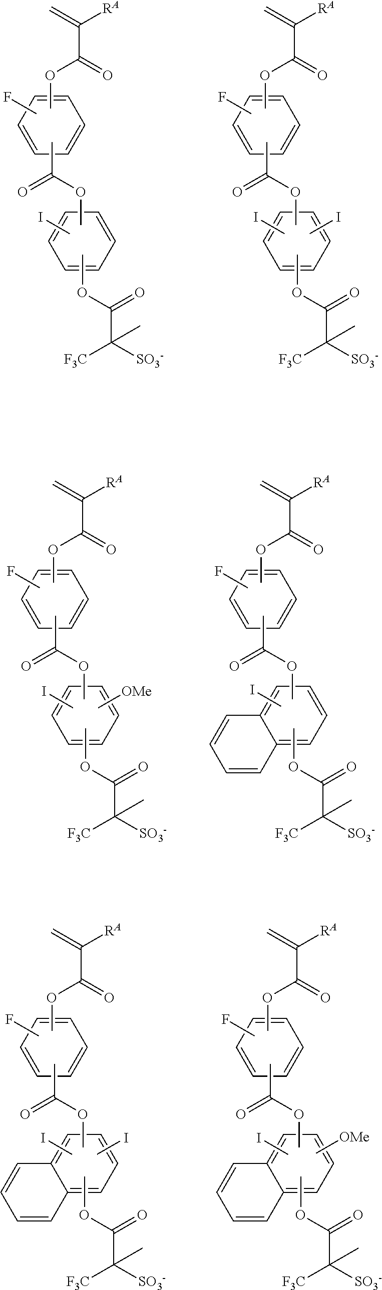

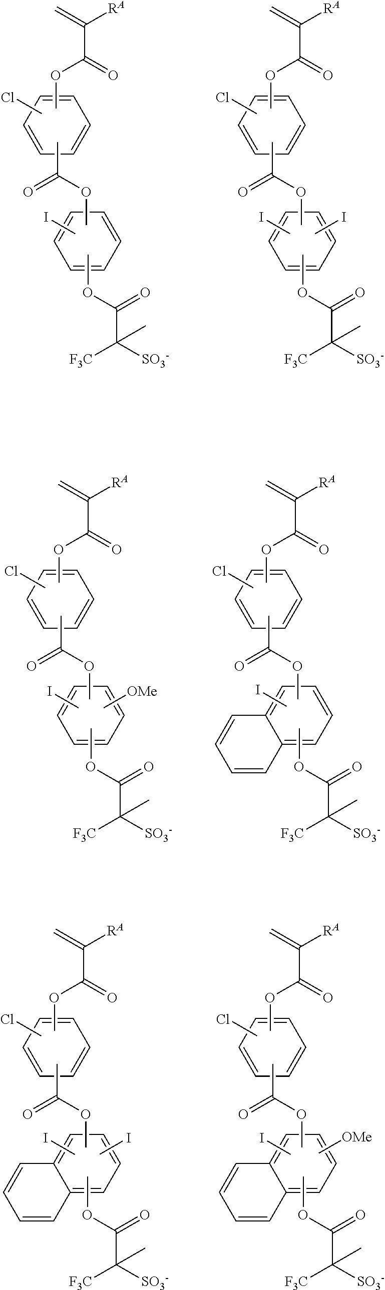

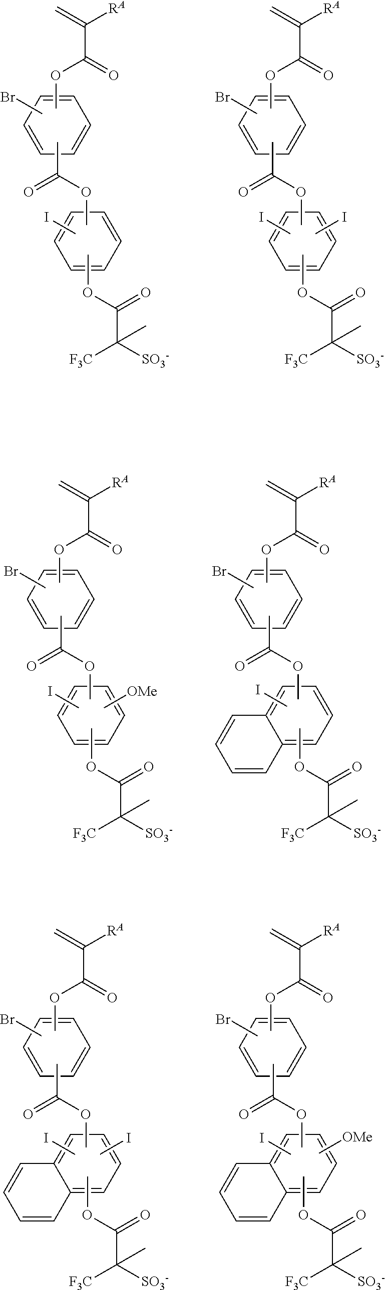

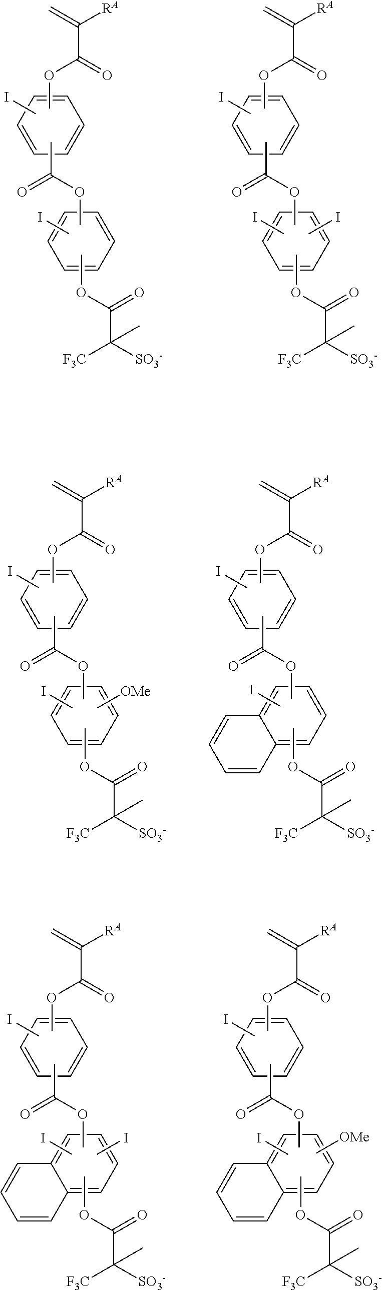

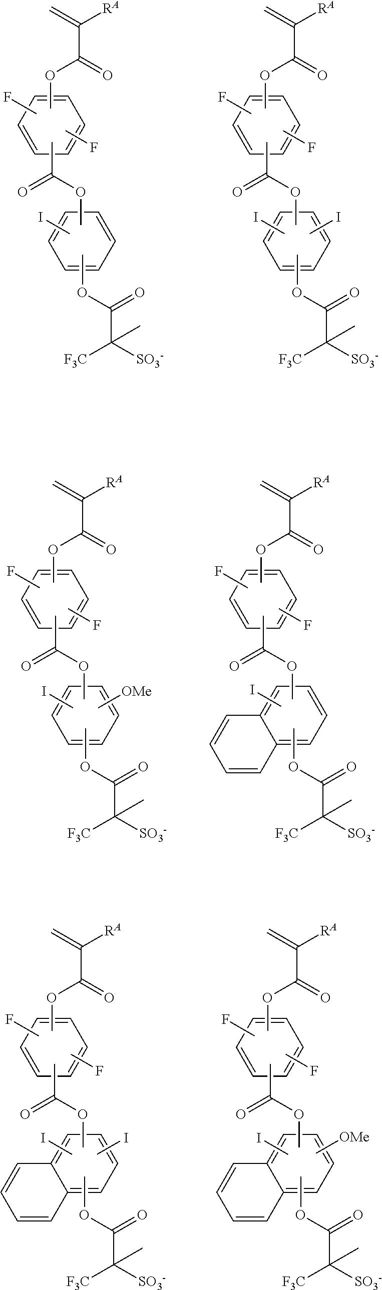

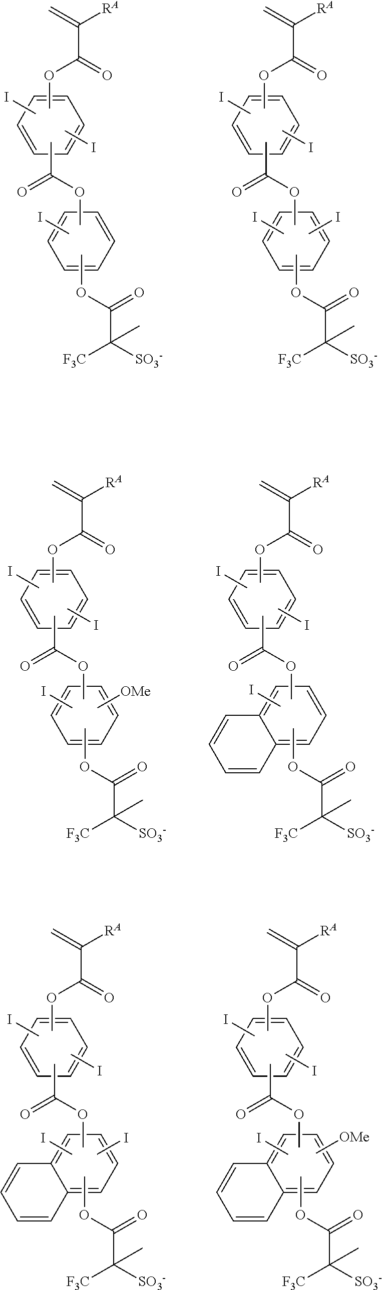

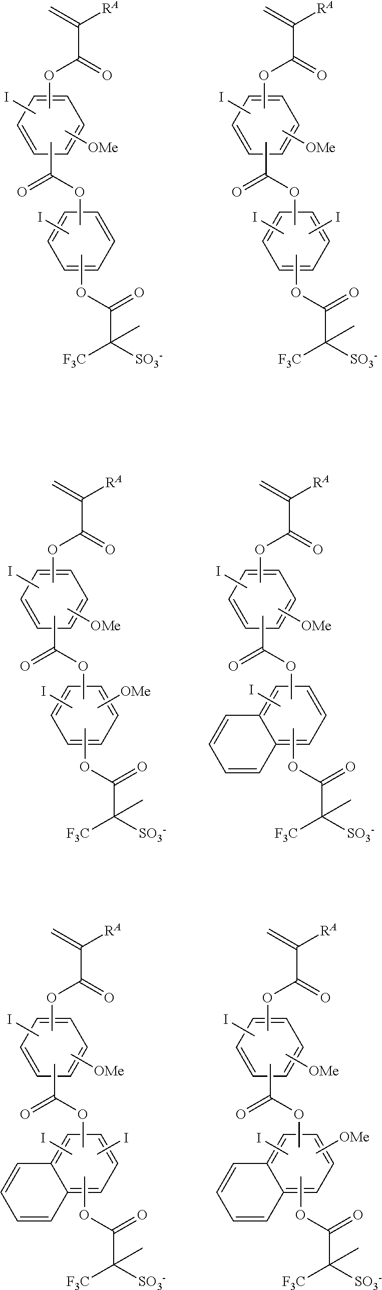

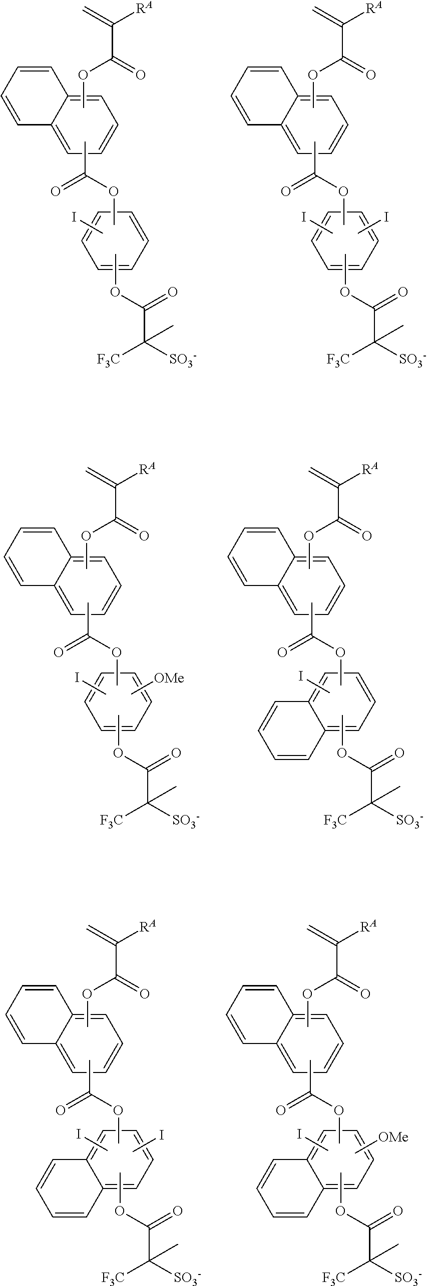

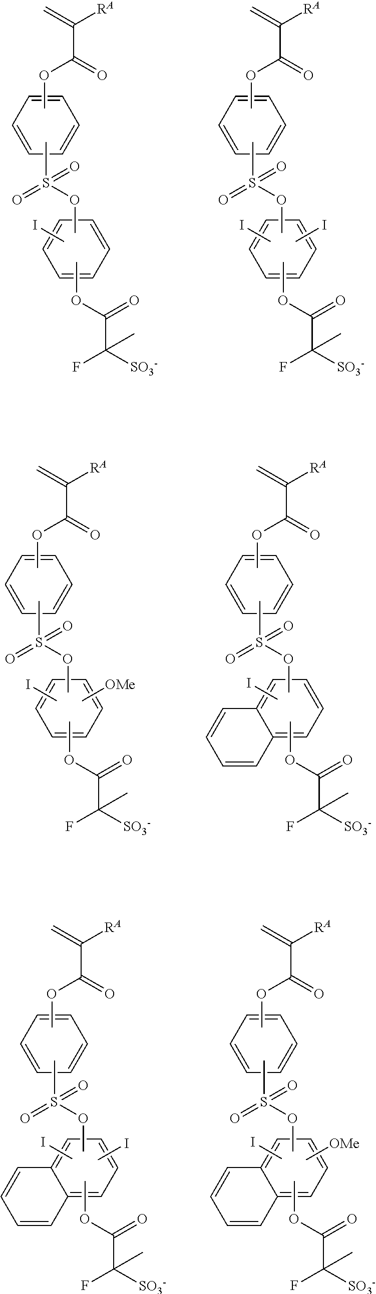

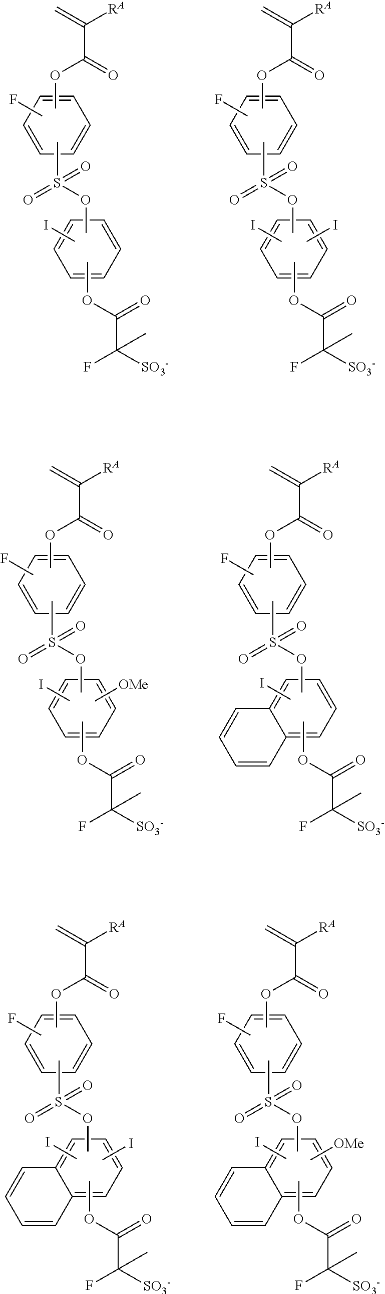

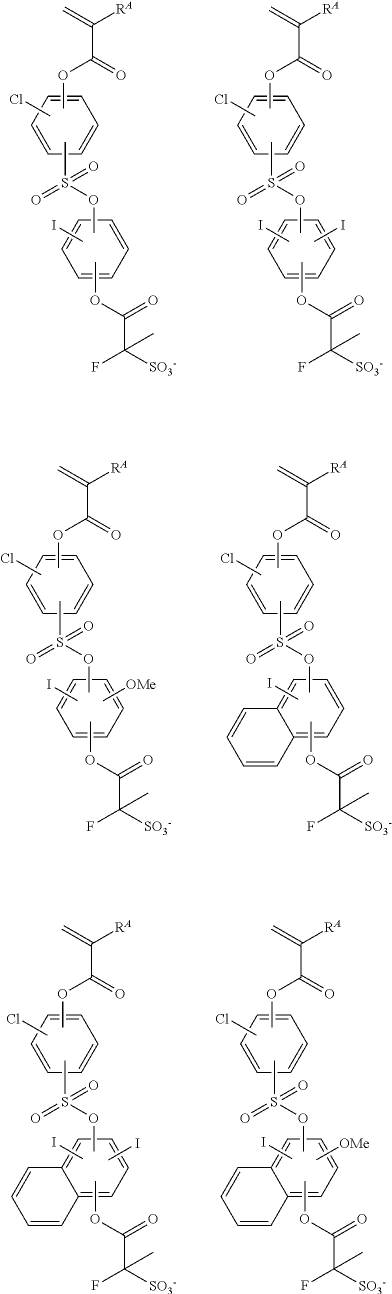

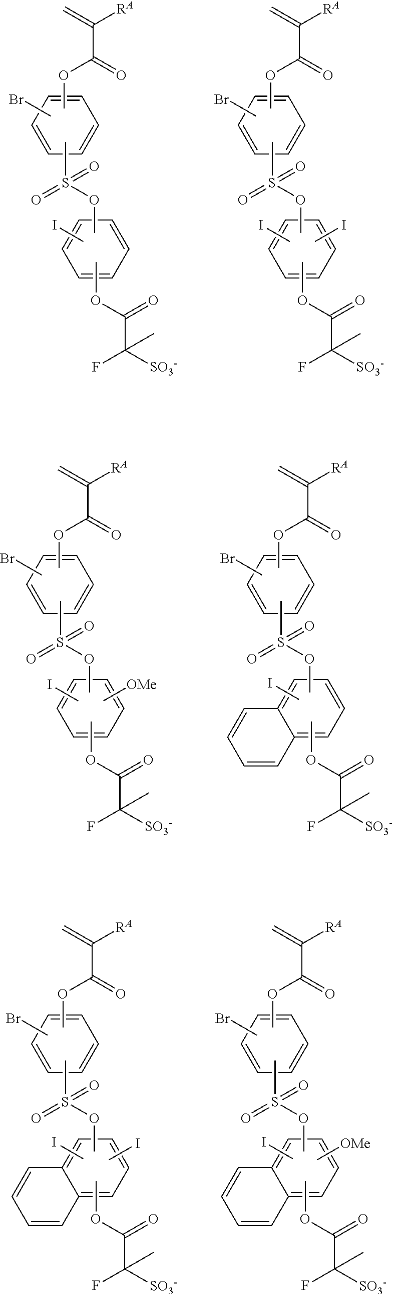

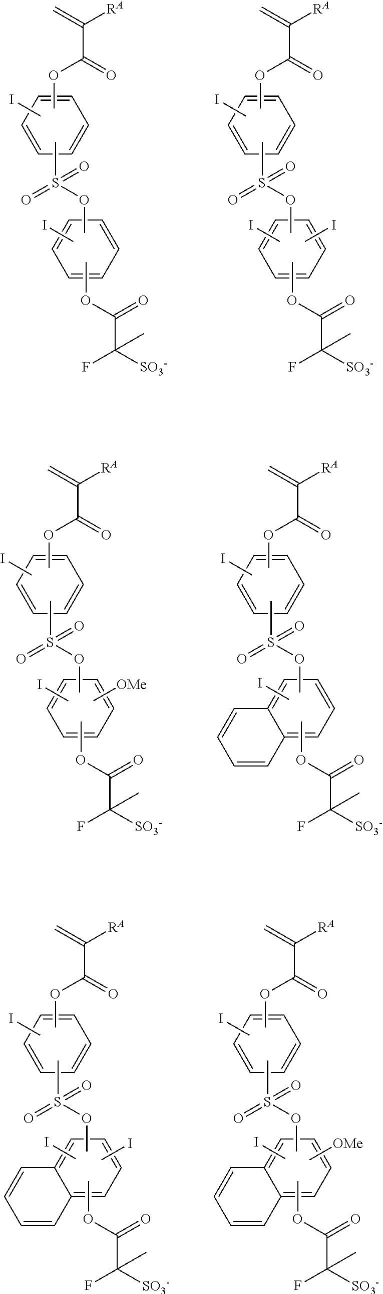

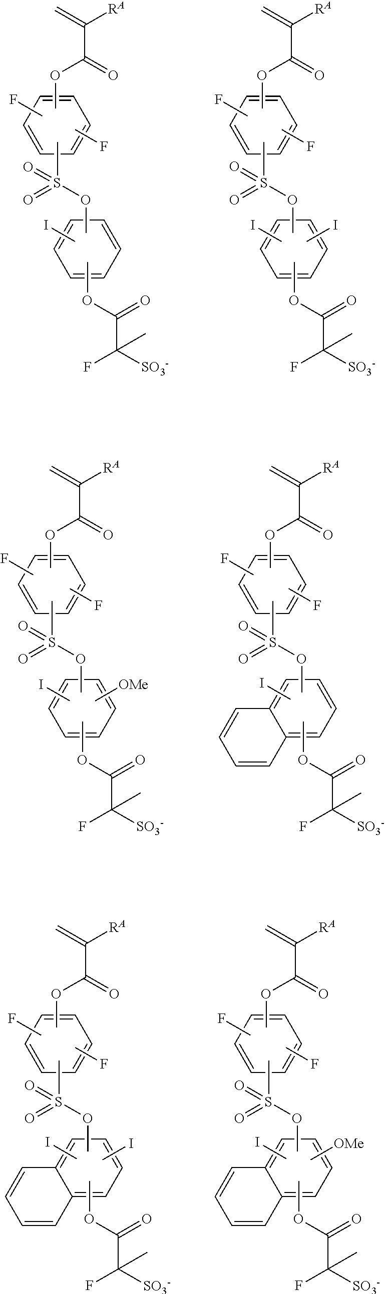

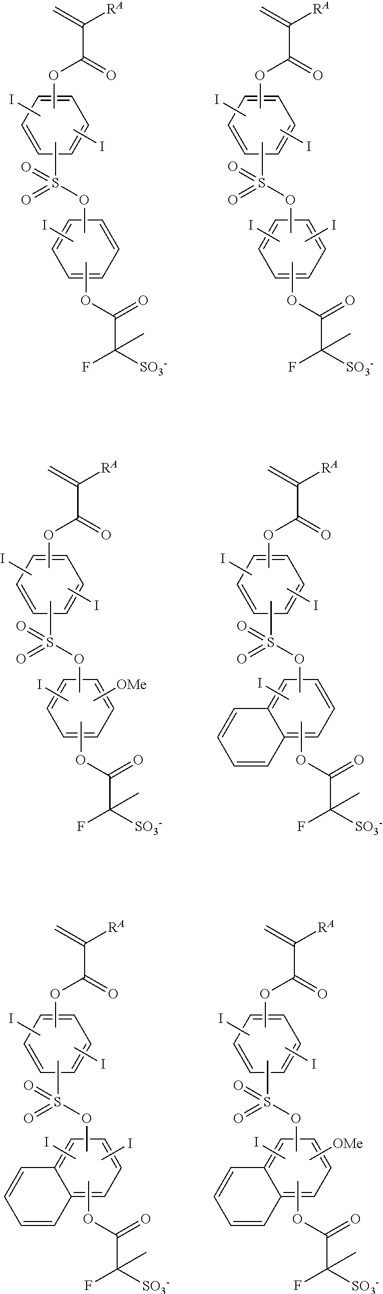

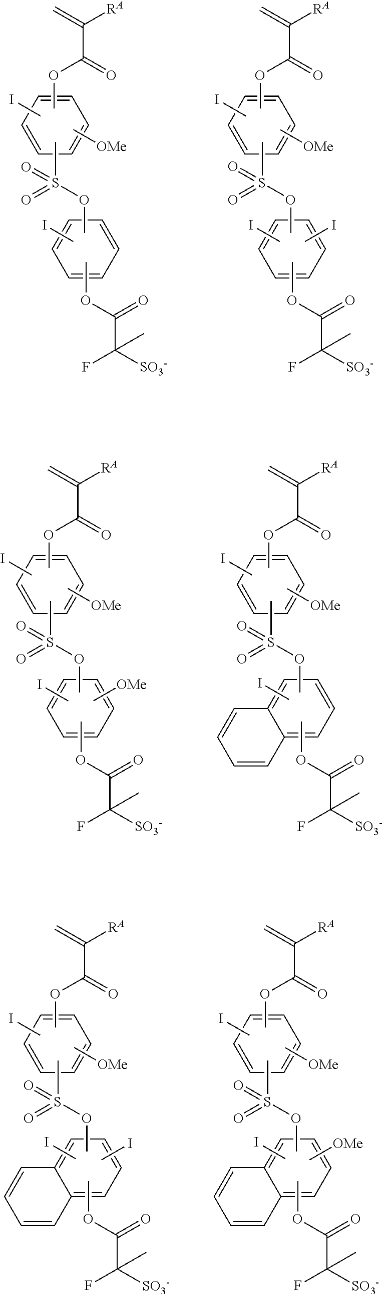

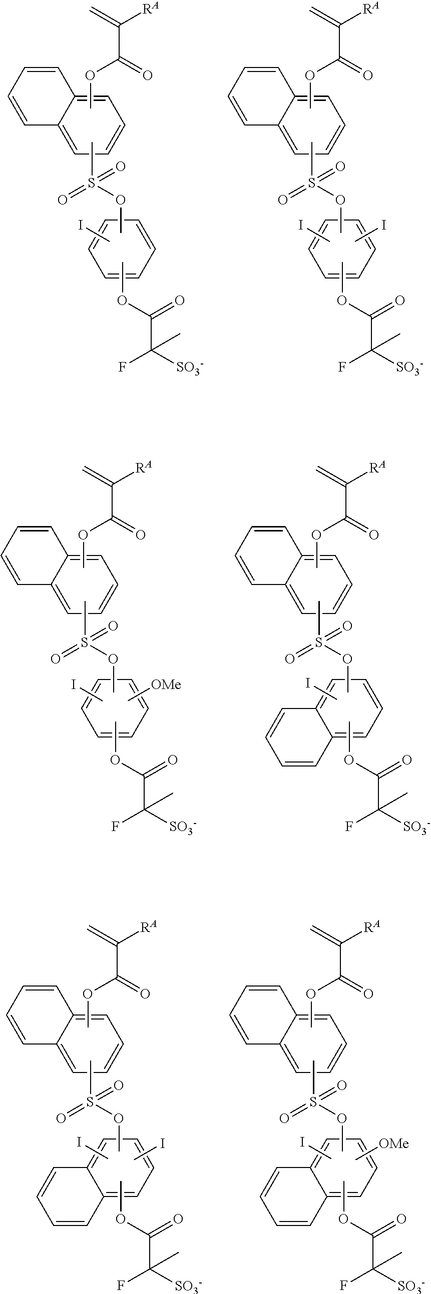

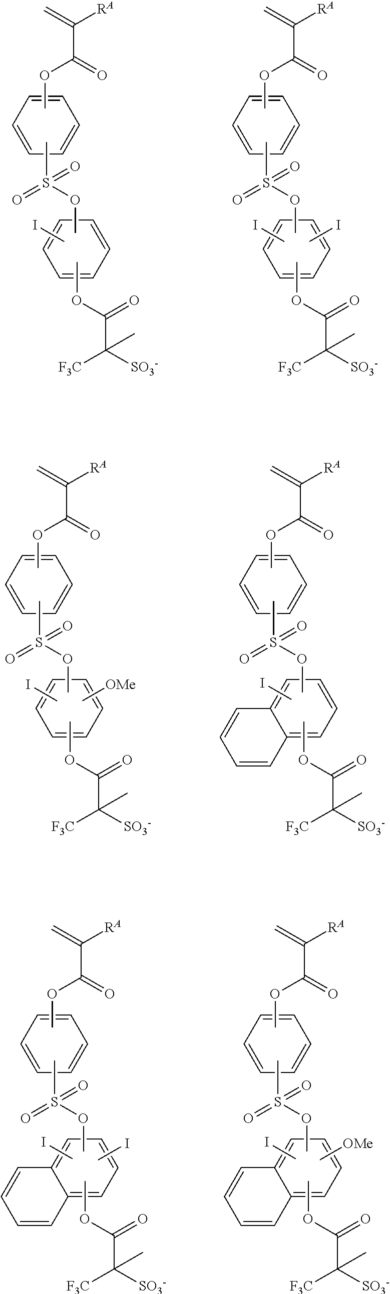

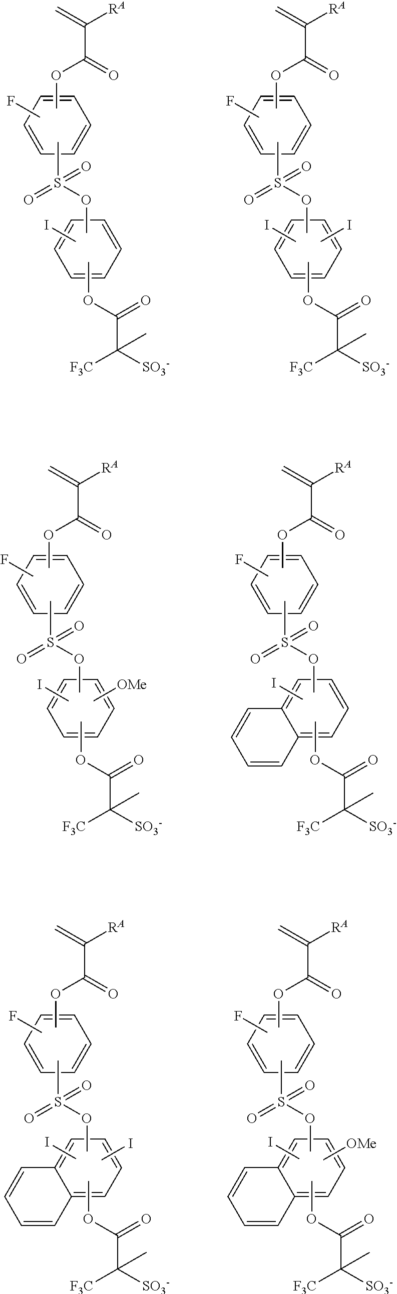

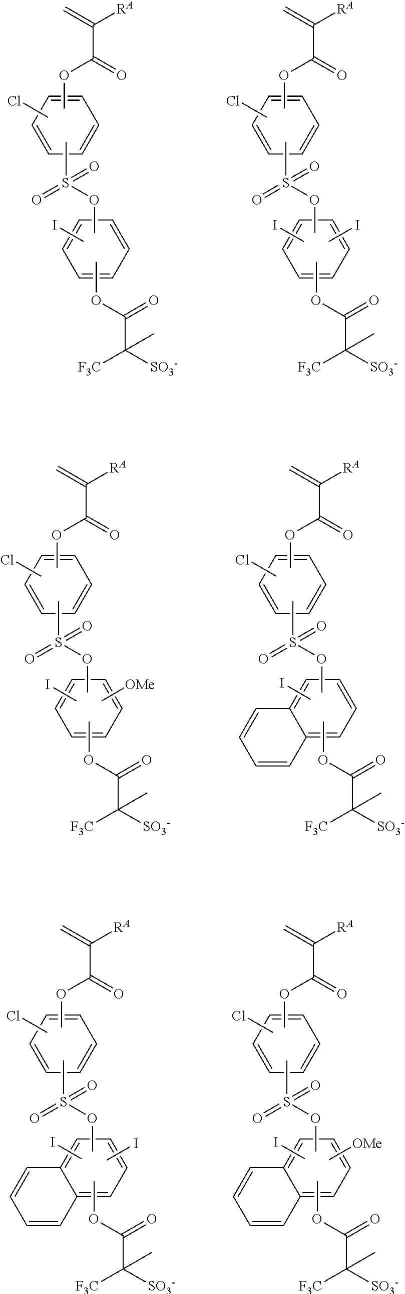

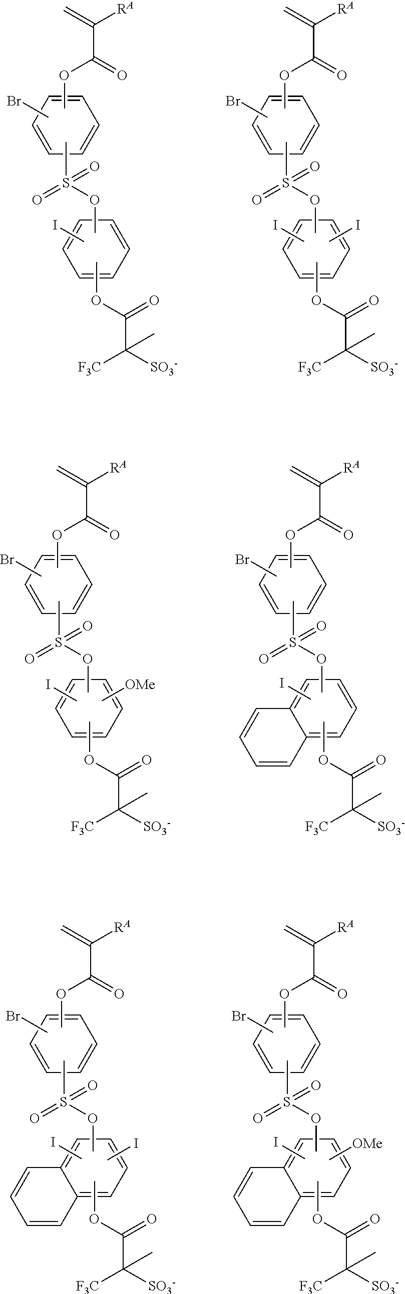

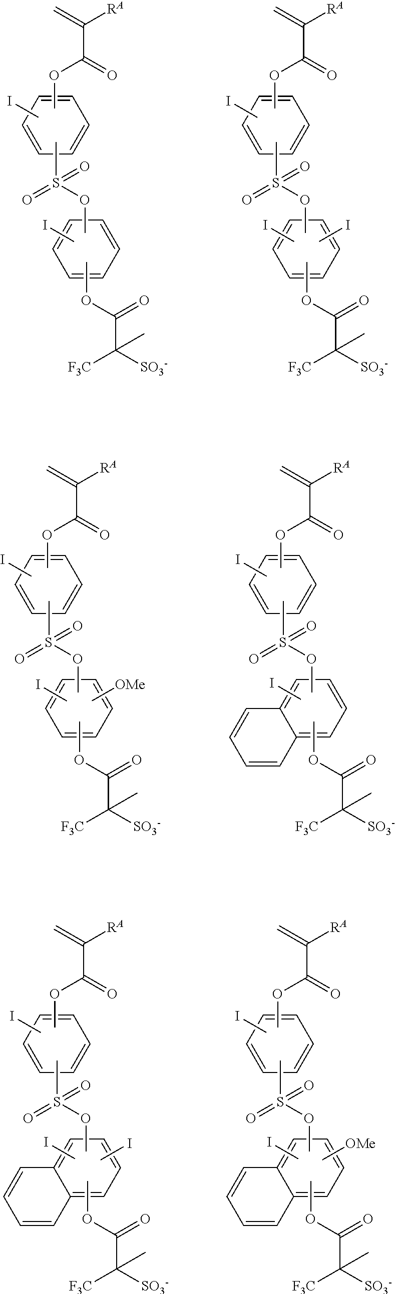

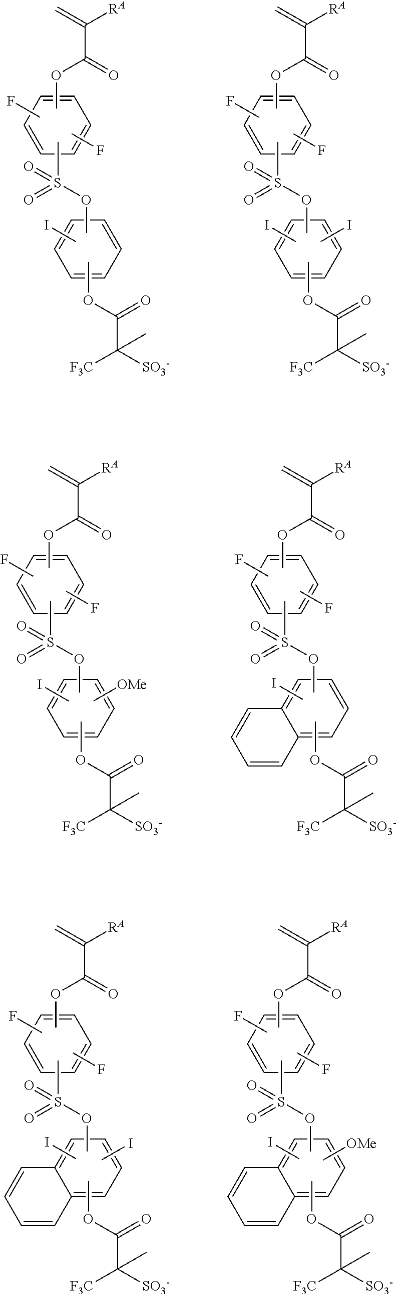

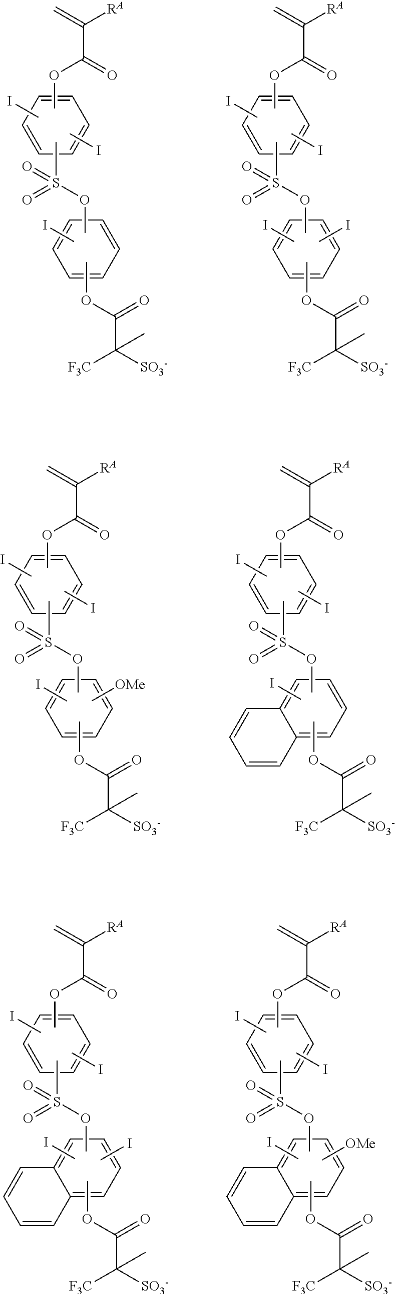

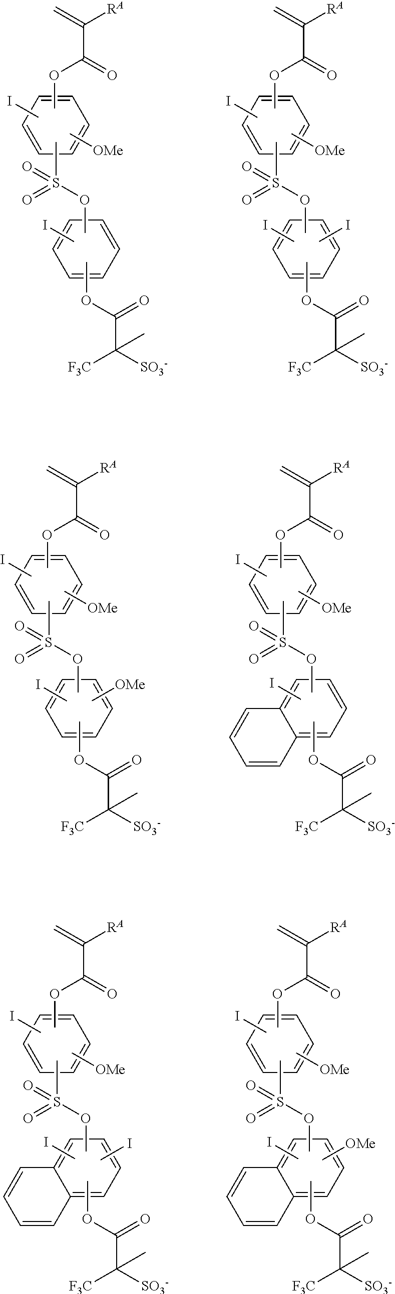

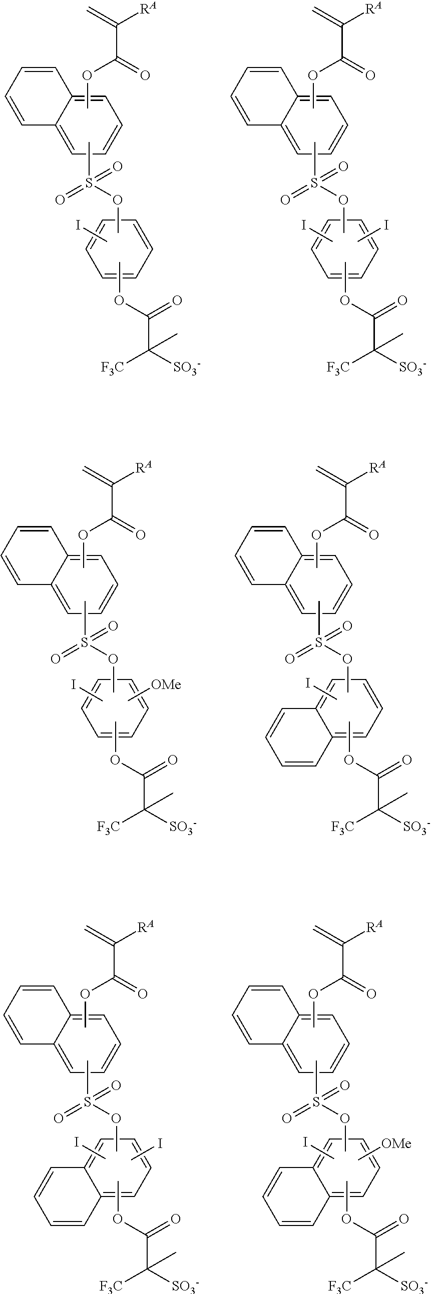

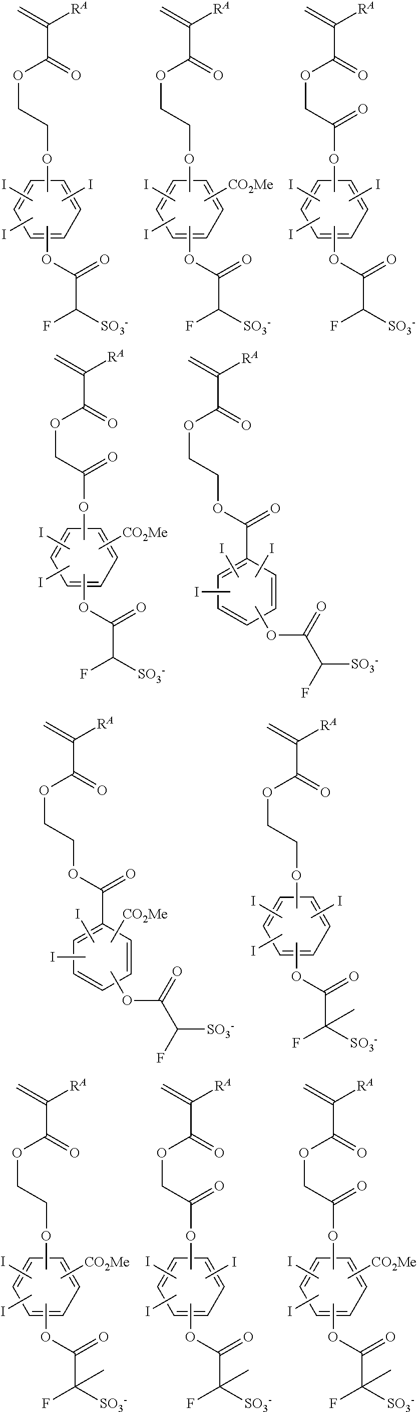

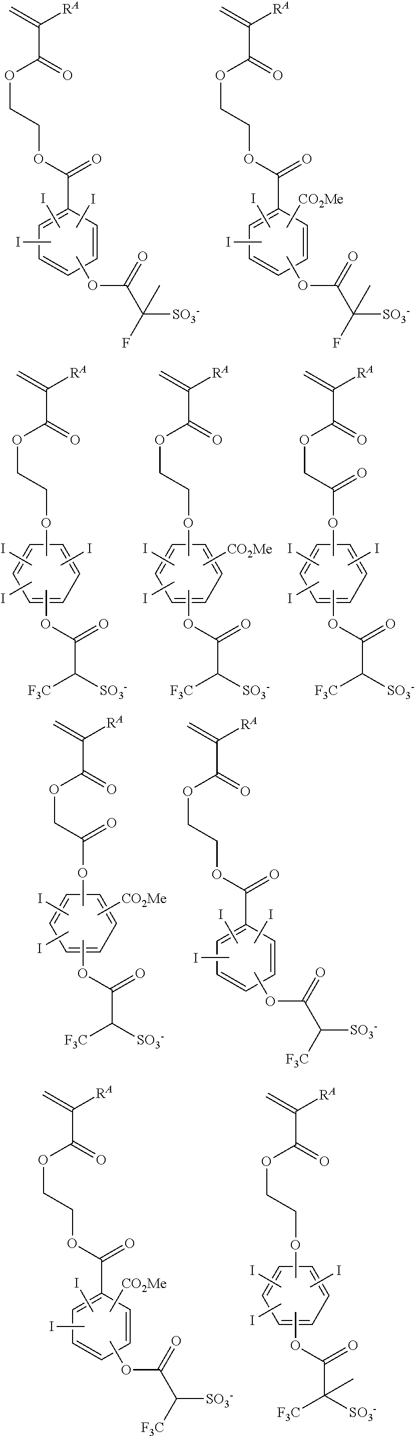

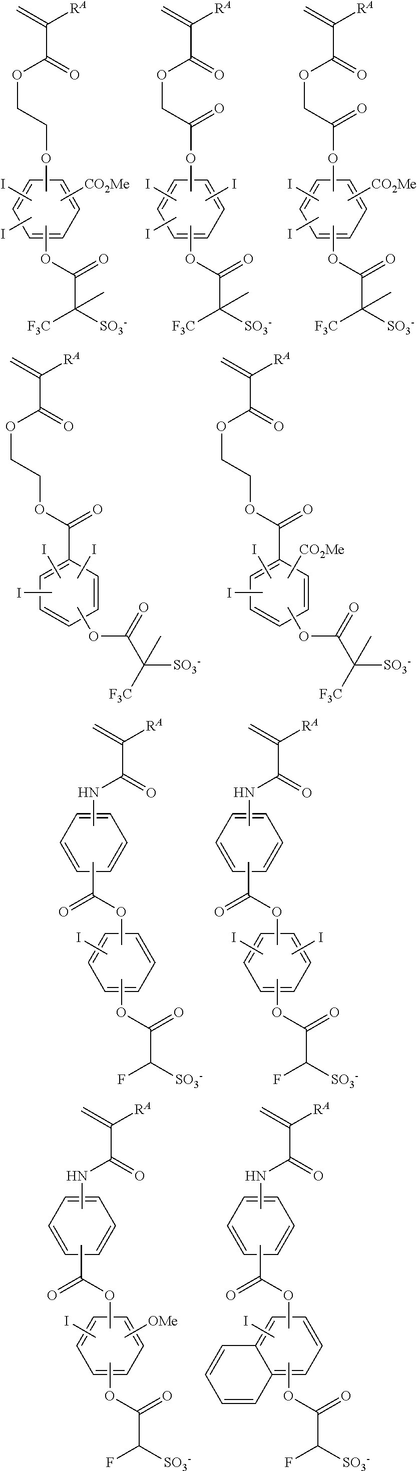

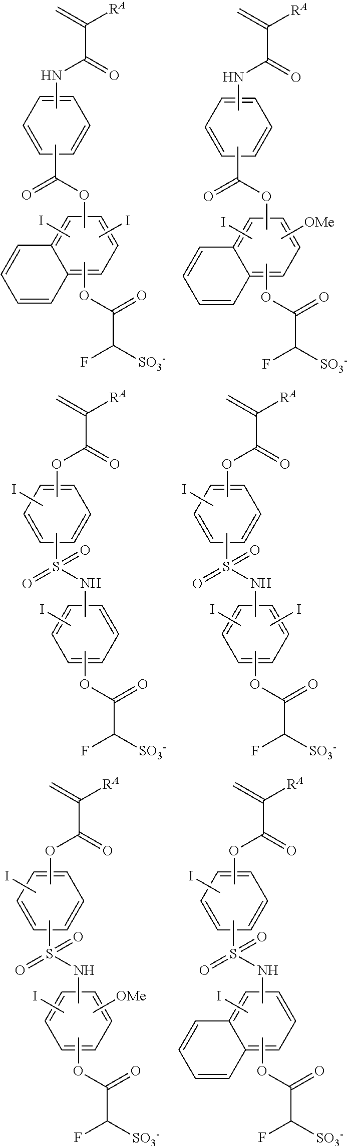

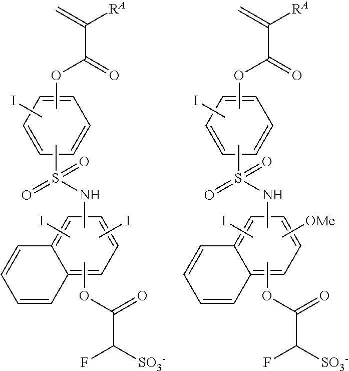

In the general formula (A), Z⁺ represents an onium cation. Z⁺ preferably represents a sulfonium cation represented by the following general formula (Z-1) or an iodonium cation represented by the following general formula (Z-2).

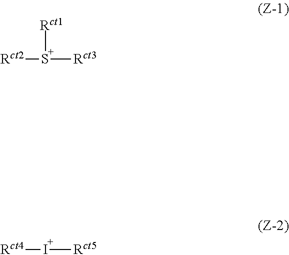

In the formulae, R^{ct1} to R^{ct5} each independently represent a halogen atom, or a hydrocarbyl group having 1 to 30 carbon atoms and optionally containing a heteroatom; and also, R^{ct1} and R^{ct2} may be bonded to each other to form a ring together with the sulfur atom to which they are bonded.

In the general formulae (Z-1) and (Z-2), R^{ct1} to R^{ct5} each independently represent a halogen atom, or a hydrocarbyl group having 1 to 30 carbon atoms and optionally containing a heteroatom.

Specific examples of the halogen atom represented by R^{ct1} to R^{ct5} include a fluorine atom, a chlorine atom, a bromine atom, and an iodine atom.

The hydrocarbyl group represented by R^{ct1} to R^{ct5} may be saturated or unsaturated, and may be linear, branched, or cyclic. Specific examples thereof include alkyl groups having 1 to 30 carbon atoms, such as methyl, ethyl, n-propyl, isopropyl, n-butyl, isobutyl, sec-butyl, and tert-butyl groups; cyclic saturated hydrocarbyl groups having 3 to 30 carbon atoms, such as cyclopropyl, cyclopentyl, cyclohexyl, cyclopropylmethyl, 4-methylcyclohexyl, cyclohexylmethyl, norbornyl, and adamantyl groups; alkenyl groups having 2 to 30 carbon atoms, such as vinyl, 1-propenyl, 2-propenyl, butenyl, and hexenyl groups; cyclic unsaturated hydrocarbyl groups having 3 to 30 carbon atoms, such as cyclohexenyl groups; aryl groups having 6 to 30 carbon atoms, such as phenyl, naphthyl, and thienyl groups; aralkyl groups having 7 to 30 carbon atoms, such as benzyl, 1-phenylethyl, and 2-phenylethyl groups; and groups obtained by combining these groups, with aryl groups being preferable. Furthermore, some or all of the hydrogen atoms of the hydrocarbyl groups may be substituted with a group containing a heteroatom such as an oxygen atom, a sulfur atom, a nitrogen atom, or a halogen atom, and some of the —CH₂— of the hydrocarbyl groups may be substituted with a group containing a heteroatom such as an oxygen atom, a sulfur atom, or a nitrogen atom, and as a result, they may contain a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group.

Furthermore, R^{ct1} and R^{ct2} may be bonded to each other to form a ring together with the sulfur atom to which they are bonded. In this case, specific examples of the ring structure include those represented by the following formulae.

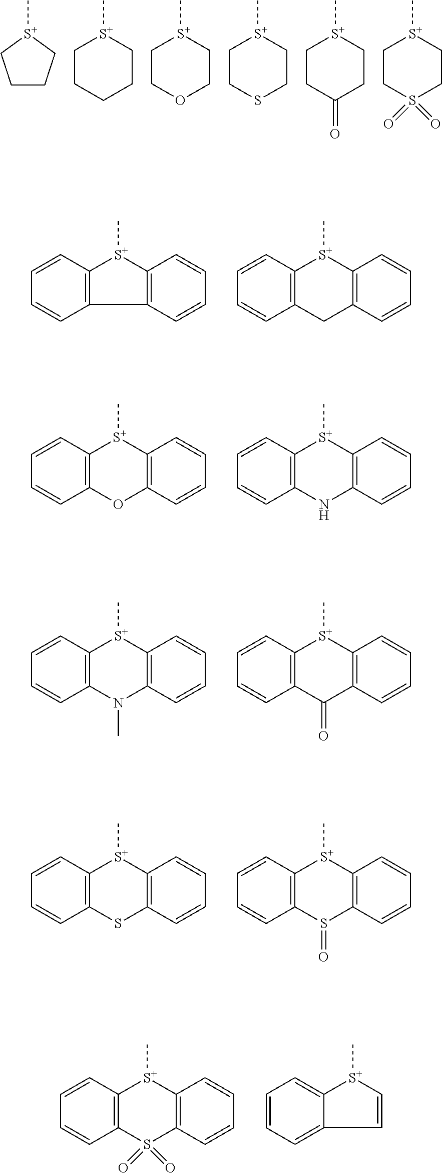

In the formulae, a broken line represents an attachment point to R^{ct3}.

Specific examples of the sulfonium cation represented by the general formula (Z-1) include those described in paragraphs [0102] to [0125] of JP2024-003744A, those described in paragraphs [0070] to [0085] of JP2023-169812A, those described in paragraphs [0044] to [0049] of WO2024/128017A1, and those described in paragraphs [0035] to [0046] of JP7491173B2, but are not limited thereto.

Specific examples of the iodonium cation represented by the general formula (Z-2) include those described in paragraph [0181] of JP2024-000259A, but are not limited thereto.

As the onium cation represented by Z⁺, a sulfonium cation represented by the following general formula (Z-3) is also preferable.

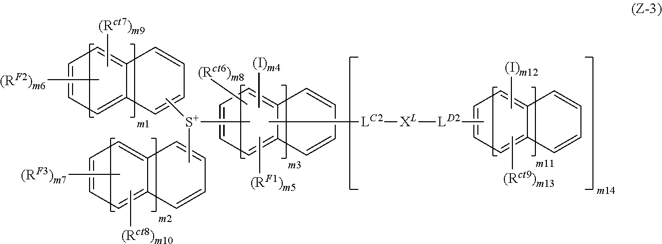

In the general formula (Z-3), “m1” represents 0 or 1. When “m1” is 0, it represents a benzene ring, and when “m1” is 1, it represents a naphthalene ring; however, from the viewpoint of solvent solubility, it is preferable that “m1” is 0 and represents a benzene ring. “m2” represents 0 or 1. When “m2” is 0, the structure is a benzene ring, and when “m2” is 1, it is a naphthalene ring; however, from the viewpoint of solvent solubility, it is preferable that “m2” is 0, giving a benzene ring. “m3” represents 0 or 1. When “m3” is 0, the structure is a benzene ring, and when “m3” is 1, it is a naphthalene ring; however, from the viewpoint of solvent solubility, it is preferable that “m3” is 0, giving a benzene ring.

In the general formula (Z-3), “m4” represents an integer from 0 to 4. The greater the number of iodine atoms in the cationic structure, the greater the absorption, particularly with respect to EUV, but there is a concern regarding precipitation in the resist composition due to poor solubility in solvents, so “m4” is preferably an integer from 0 to 3, and more preferably 0, 1, or 2.

In the general formula (Z-3), “m5” represents an integer from 0 to 4. From the viewpoint of raw material procurement, “m5” is preferably an integer from 0 to 3, and more preferably 0, 1, or 2. “m6” represents an integer from 0 to 6. From the viewpoint of raw material procurement, “m6” is preferably an integer from 0 to 3, and more preferably 0, 1, or 2. “m7” represents an integer from 0 to 6. From the viewpoint of raw material procurement, “m7” is preferably an integer from 0 to 3, and more preferably 0, 1, or 2.

In the general formula (Z-3), “m8” represents 0, 1, or 2. From the viewpoint of raw material procurement, “m8” is preferably 0 or 1. “m9” represents 0, 1, or 2. From the viewpoint of raw material procurement, “m9” is preferably 0 or 1. “m10” represents 0, 1, or 2. From the viewpoint of raw material procurement, “m10” is preferably 0 or 1.

In the general formula (Z-3), “m11” represents 0 or 1. When “m11” is 0, it represents a benzene ring, and when “m11” is 1, it represents a naphthalene ring; however, from the viewpoint of solvent solubility, it is preferable that “m11” is 0 and represents a benzene ring.

In the general formula (Z-3), “m12” represents an integer from 0 to 4. The greater the number of iodine atoms in the cationic structure, the greater the absorption, particularly with respect to EUV, but there is a concern regarding precipitation in the resist composition due to poor solubility in solvents, so “m12” is preferably an integer from 0 to 3, and more preferably 0, 1, or 2.

In the general formula (Z-3), “m13” represents 0, 1, or 2. From the viewpoint of raw material procurement, “m13” is preferably 0 or 1. “m14” represents 0, 1, or 2. From the viewpoint of synthesis, “m14” is preferably 0 or 1.

However, when “m1” is 0, 0≤m6+m9≤4, and when “m1” is 1, 0≤m6+m9≤6. When “m2” is 0, 0≤m7+m10≤4, and when “m2” is 1, 0≤m7+m10≤6. When “m3” is 0, 1≤m4+m5+m8+m14≤4, and when “m3” is 1, 1≤m4+m5+m8+m14≤6. When “m11” is 0, 0≤m12+m13≤4, and when “m11” is 1, 0≤m12+m13≤6. Furthermore, 1≤m4+m12.

In the general formula (Z-3), R^{F1} to R^{F3} each independently represent a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms. Of these, a trifluoromethyl group, a trifluoromethoxy group, or a trifluorothiomethoxy group is preferable. When “m5” is 2 or more, each R^{F1} may be the same as or different from each other, when “m6” is 2 or more, each R^{F2} may be the same as or different from each other, and when “m7” is 2 or more, each R^{F3} may be the same as or different from each other.

In the general formula (Z-3), R^{ct6} to R^{ct9} represent a halogen atom other than an iodine atom and a fluorine atom, a nitro group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom. The hydrocarbyl groups and the hydrocarbyl moieties of the hydrocarbyloxy and the hydrocarbylthio groups may be saturated or unsaturated, and may be linear, branched, or cyclic. Specific examples thereof include the same ones as those given as examples of the hydrocarbyl group represented by R¹ in the explanation of the general formula (A). Furthermore, some or all of the hydrogen atoms in the hydrocarbyl groups and the hydrocarbyl moieties of the hydrocarbyloxy and the hydrocarbylthio groups may be substituted with a group containing a heteroatom such as an oxygen atom, a sulfur atom, a nitrogen atom or a halogen atom, and some of the —CH₂— of the hydrocarbyl groups may be substituted with a group containing a heteroatom such as an oxygen atom, a sulfur atom, or a nitrogen atom, and as a result, they may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group.

Furthermore, when “m8” is 2, two R^{ct6} may be the same as or different from each other, and two R^{ct6} may be bonded to each other to form a ring together with the carbon atom to which they are bonded, when “m9” is 2, two R^{ct7} may be the same as or different from each other, and two R^{ct7} may be bonded to each other to form a ring together with the carbon atoms to which they are bonded, when “m10” is 2, two R^{ct8} may be the same as or different from each other, and two R^{ct8} may be bonded to each other to form a ring together with the carbon atoms to which they are bonded, and when “m13” is 2, two R^{ct9} may be the same as or different from each other, and two R^{ct9} may be bonded to each other to form a ring together with the carbon atoms to which they are bonded. Specific examples of the ring formed in this case include a cyclopropane ring, a cyclobutane ring, a cyclopentane ring, a cyclohexane ring, a norbornane ring, and an adamantane ring. Furthermore, some or all of the hydrogen atoms in the ring may be substituted with a group containing a heteroatom such as an oxygen atom, a sulfur atom, a nitrogen atom, or a halogen atom, and some of the —CH₂— in the ring may be substituted with a group containing a heteroatom such as an oxygen atom, a sulfur atom, or a nitrogen atom, and as a result, it may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group.

Furthermore, the aromatic rings directly bonded to S⁺ in the sulfonium cation represented by the general formula (Z-3) may be bonded to each other to form a ring together with S⁺. In this case, specific examples of the ring structure include those represented by the following formulae.

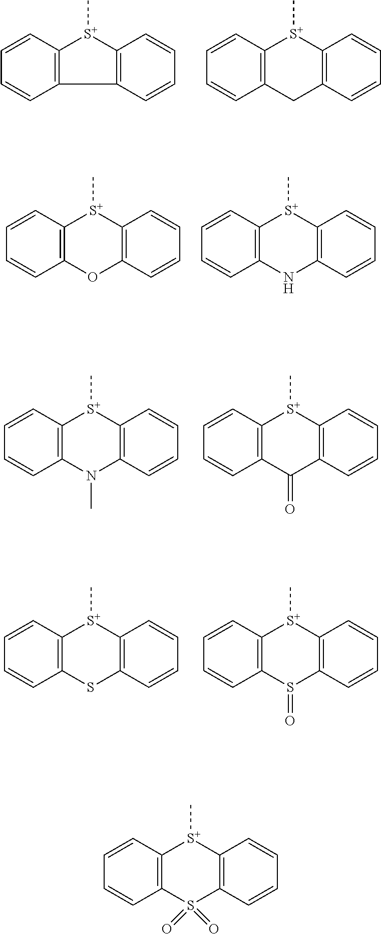

In the formulae, a broken line represents an attachment point.

In the general formula (Z-3), L^{C2} and L^{D2} each independently represent a single bond, an ether bond, an ester bond, an amide bond, a sulfonate ester bond, a sulfonate amide bond, a carbonate bond, or a carbamate bond. Of these, L^{C2} preferably represents a single bond, an ether bond, an ester bond, or a sulfonate ester bond, and more preferably an ester bond or a sulfonate ester bond. L^{D2} preferably represents a single bond, an ether bond, or an ester bond, and more preferably a single bond.

In the general formula (Z-3), X^{L} represents a single bond, or a hydrocarbylene group having 1 to 40 carbon atoms and optionally containing a heteroatom. The hydrocarbylene group may be linear, branched, or cyclic, and specific examples thereof include an alkanediyl group, a cyclic saturated hydrocarbylene group, and an arylene group. Specific examples of the heteroatom include an oxygen atom, a nitrogen atom, and a sulfur atom.

Specific examples of the hydrocarbylene group having 1 to 40 carbon atoms optionally containing a heteroatom represented by X^{L} include, but are not limited to, the same as those listed given for X^{L1} and X^{L2} in the general formula (A).

The sulfonium cation represented by the general formula (Z-3) is preferably one represented by the following general formula (Z-3-1).

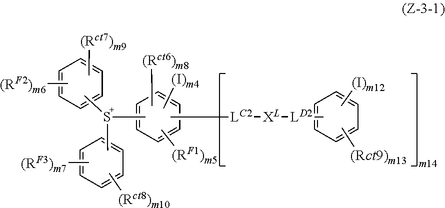

In the formula, “m4” to “m10”, “m12” to “m14”, R^{F1} to R^{F3}, R^{ct6} to R^{ct9}, L^{C2}, L^{D2}, and X^{L} are as defined above.

The sulfonium cation represented by the general formula (Z-3-1) is preferably one represented by the following general formula (Z-3-2).

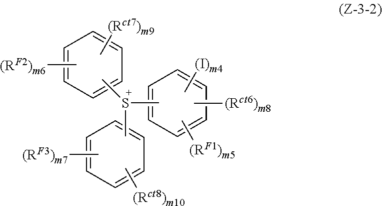

In the formula, “m4” to “m10”, R^{F1} to R^{F3}, and R^{ct6} to R^{ct8} are as defined above.

Specific examples of the sulfonium cation represented by the general formula (Z-3) include, but are not limited to, the following. In the following formulae, Me represents a methyl group.

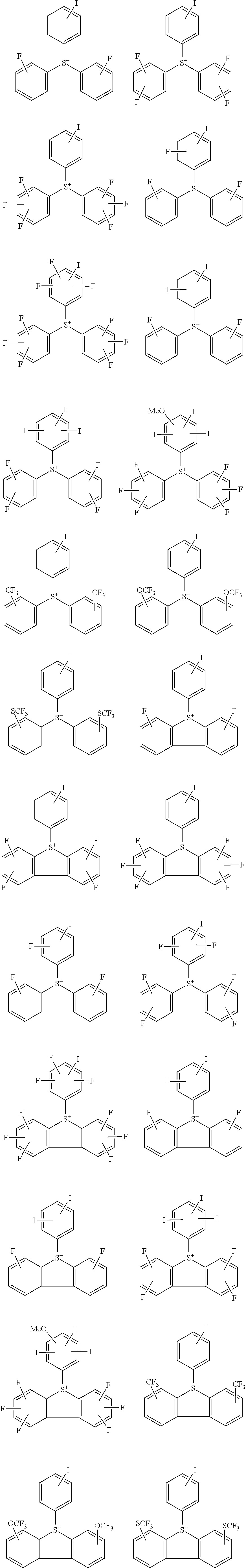

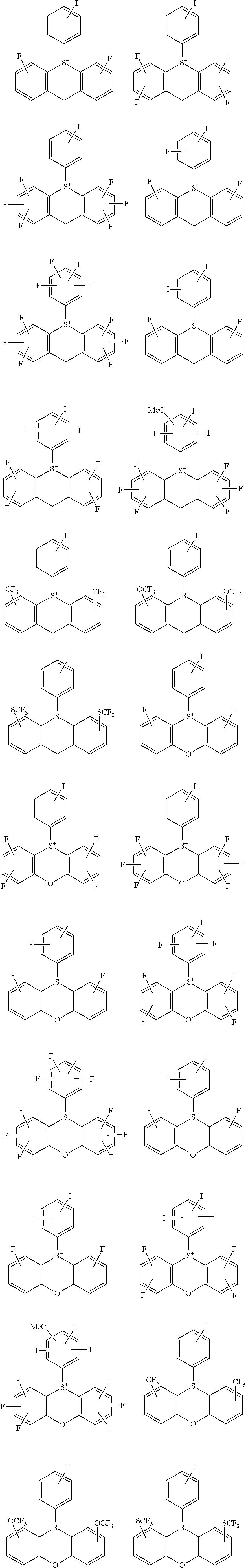

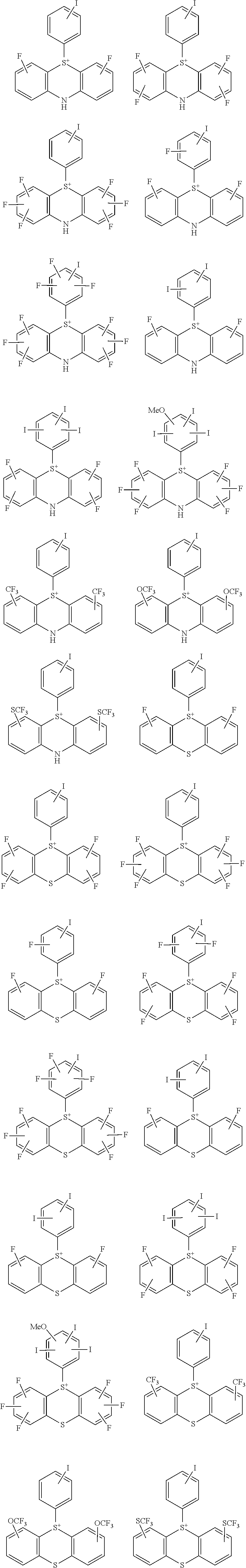

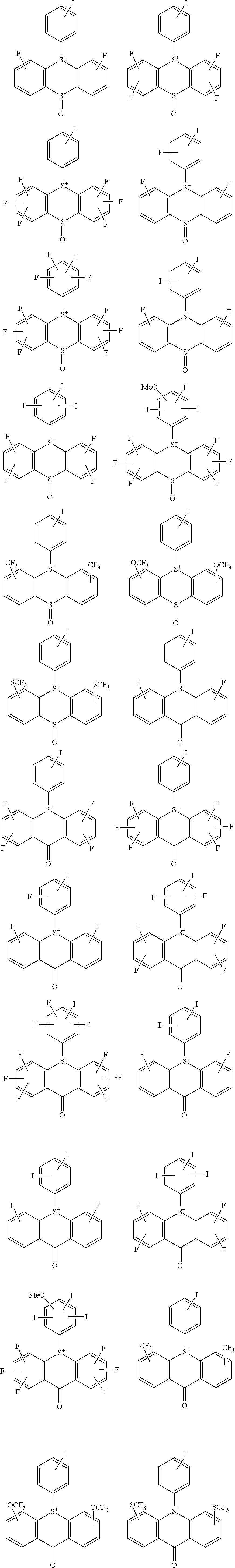

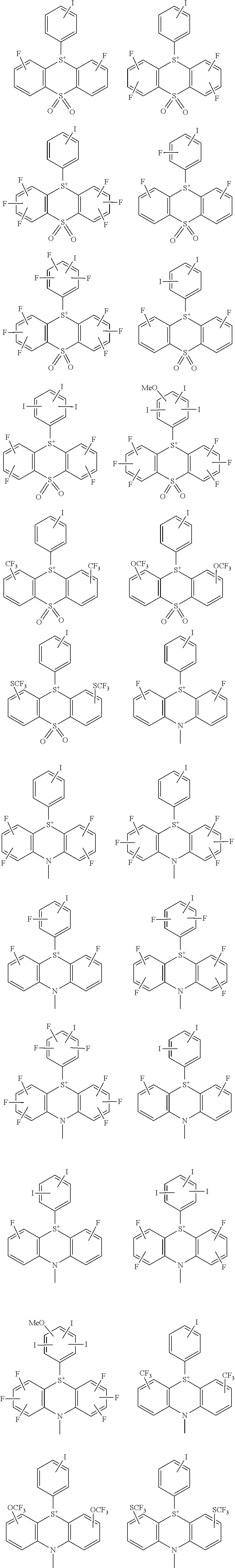

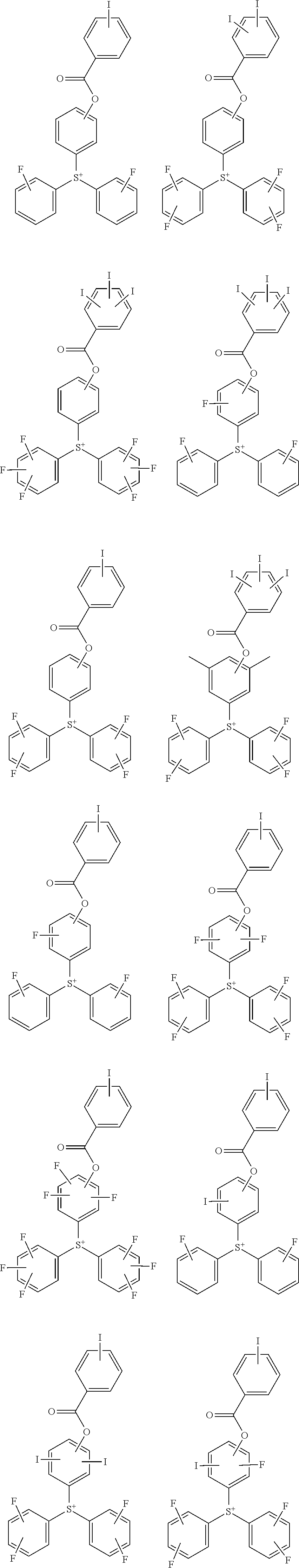

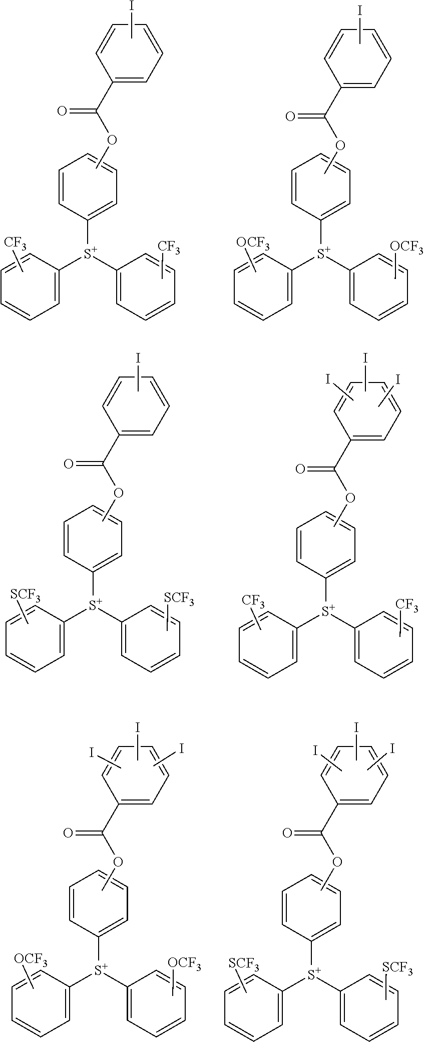

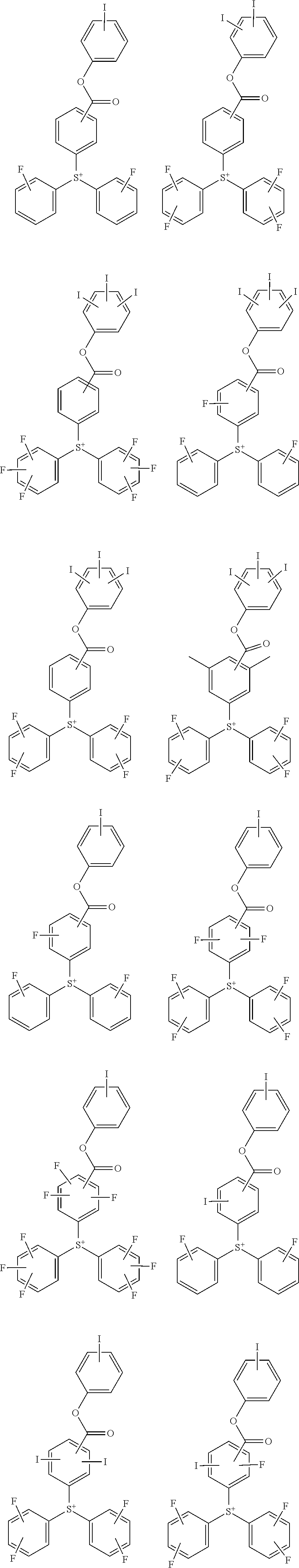

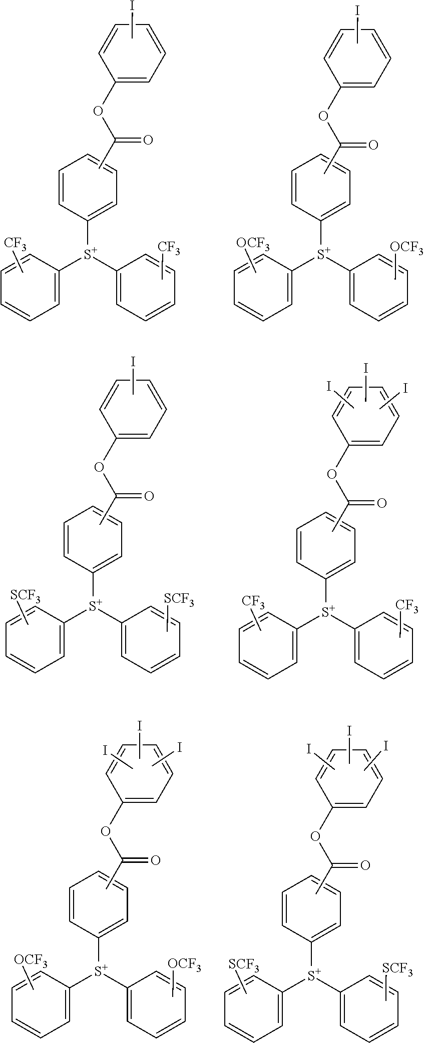

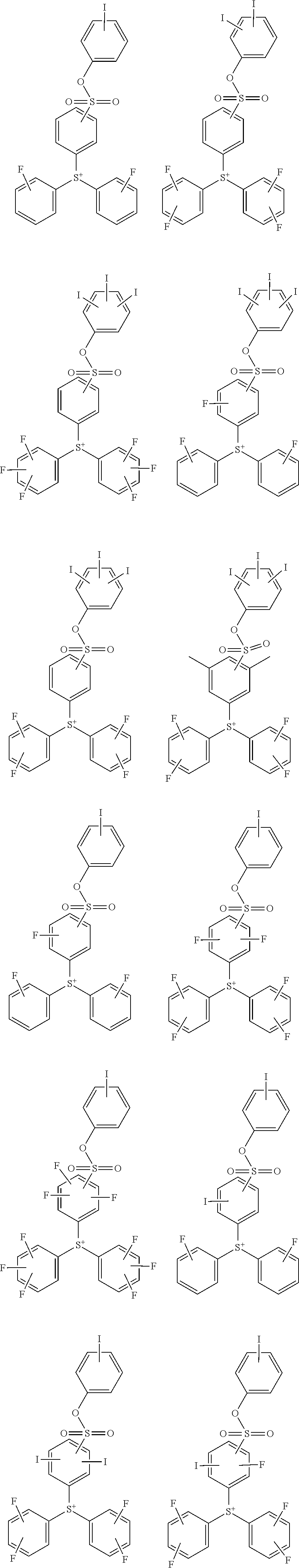

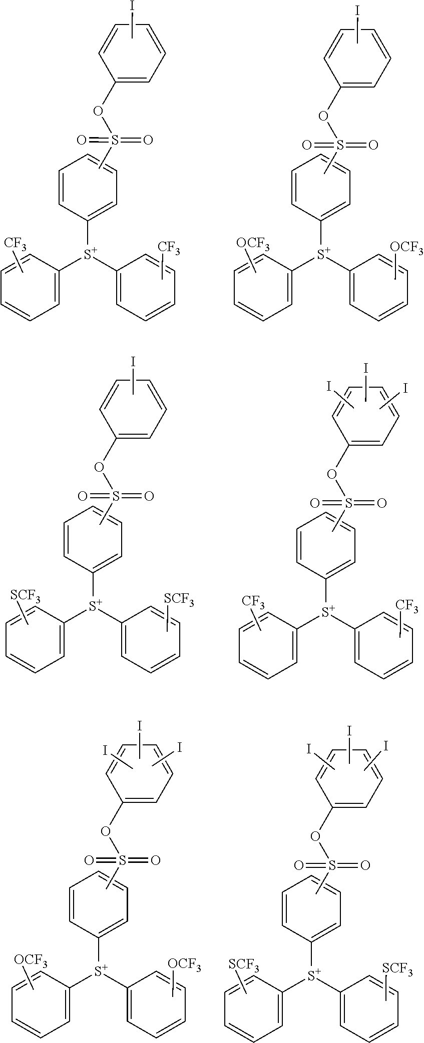

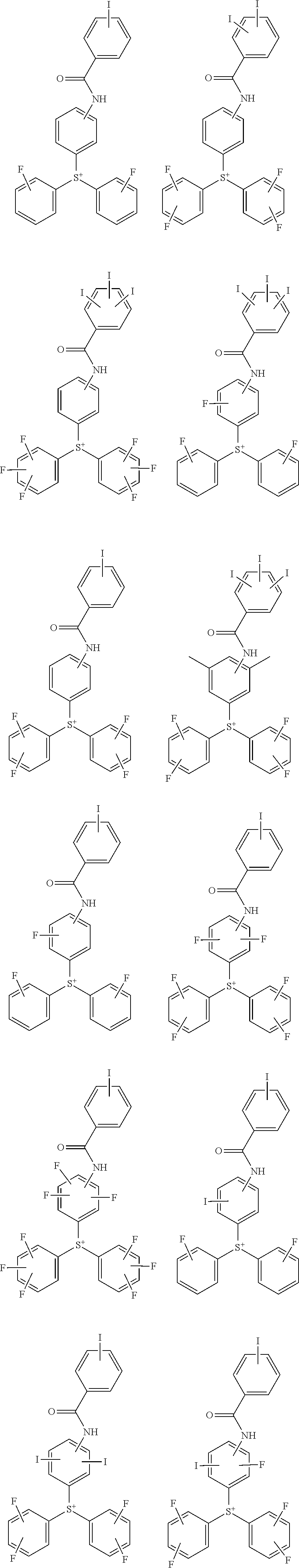

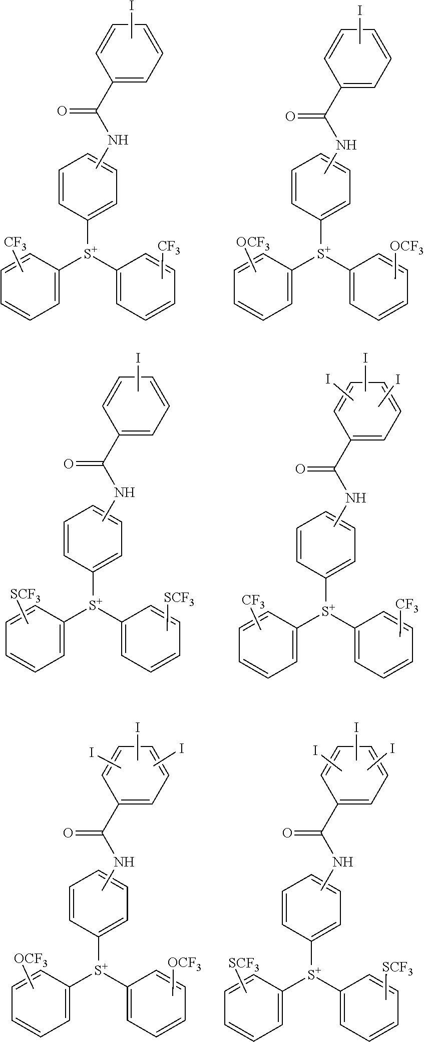

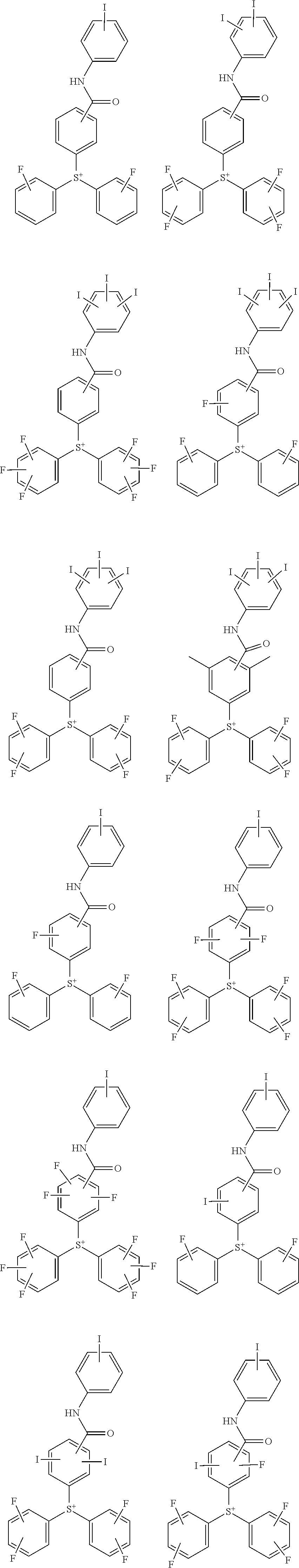

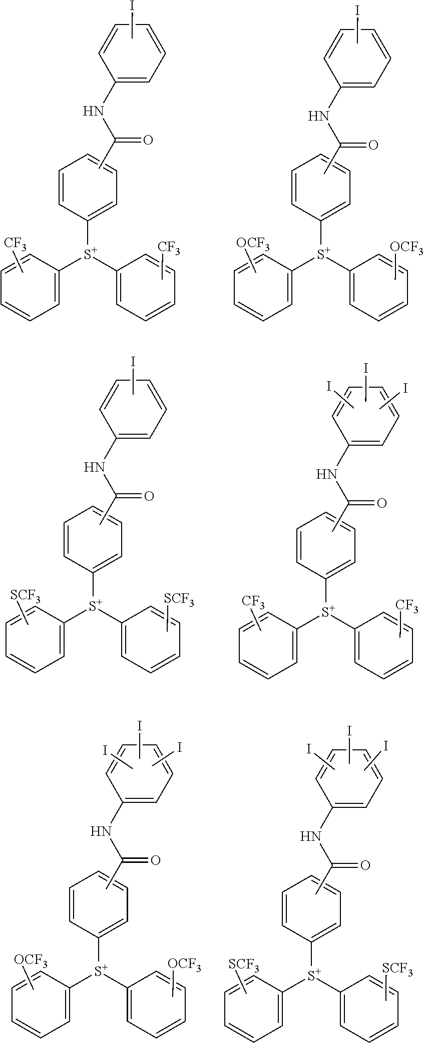

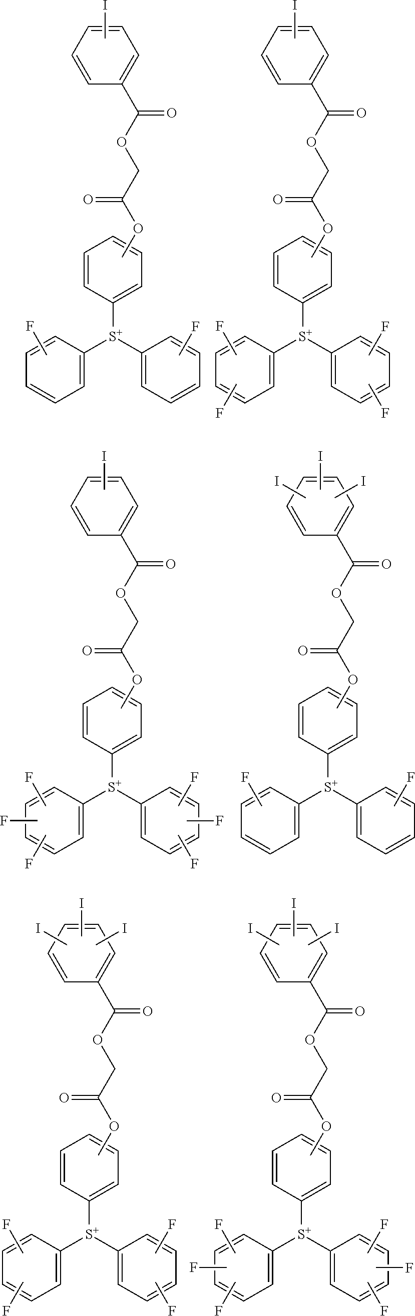

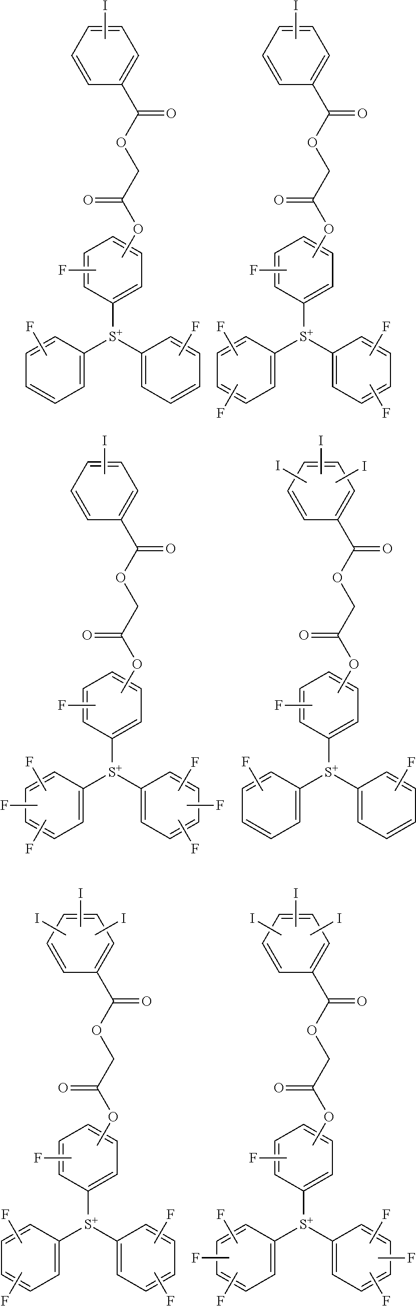

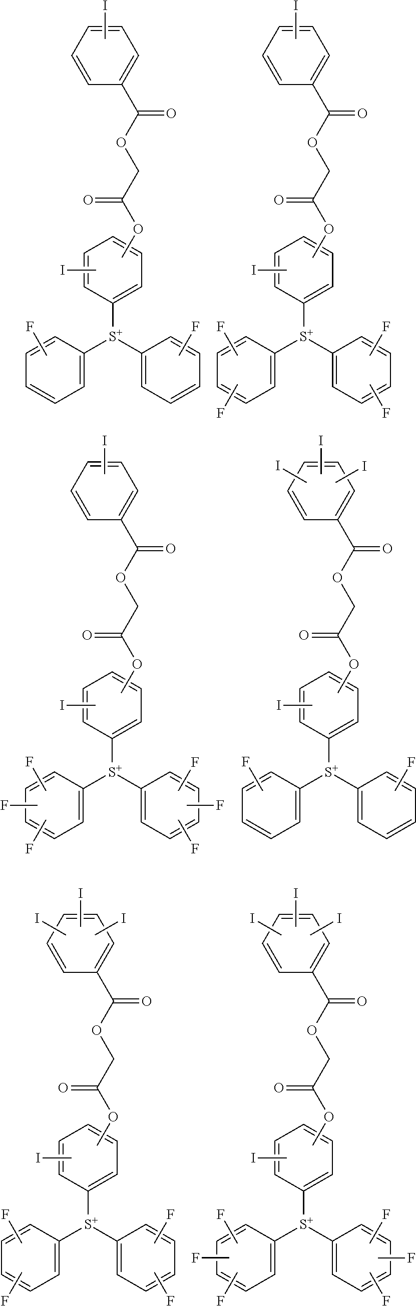

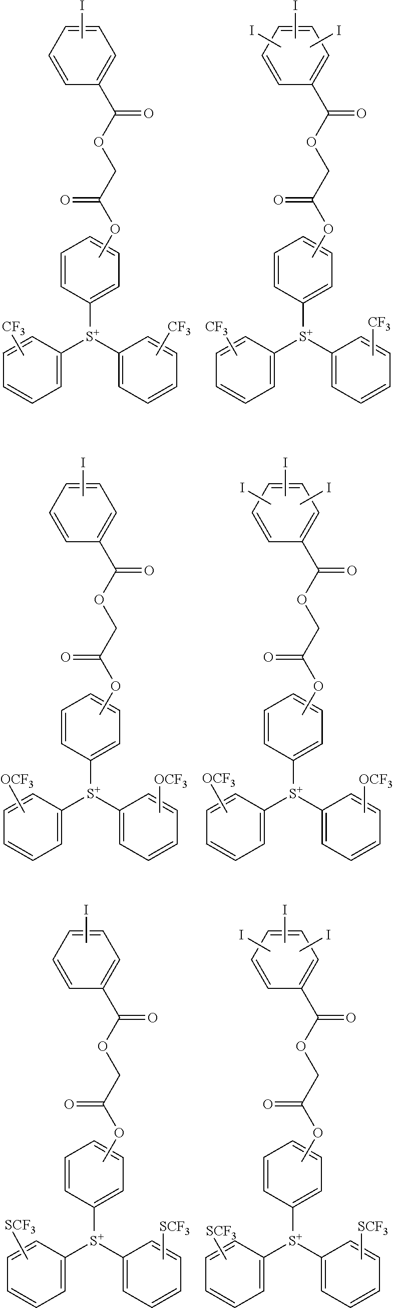

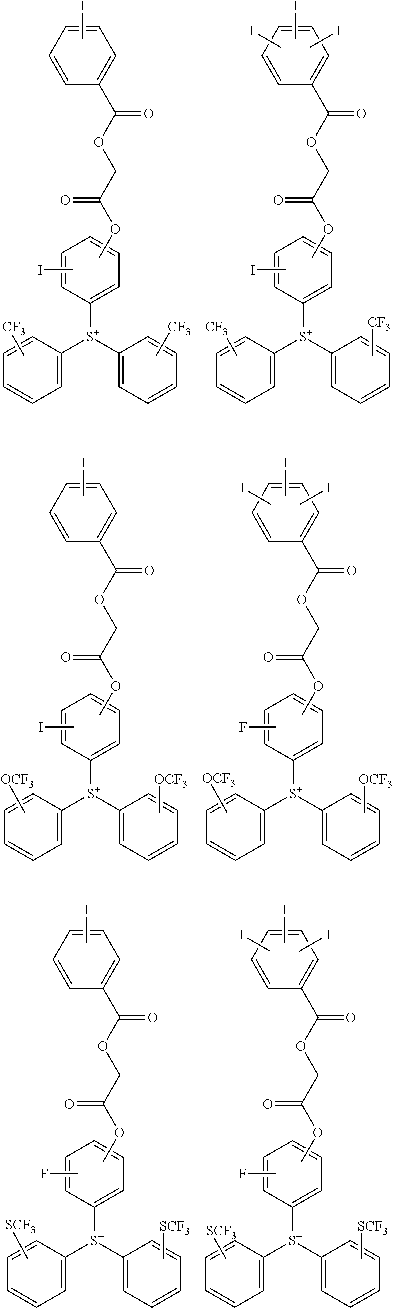

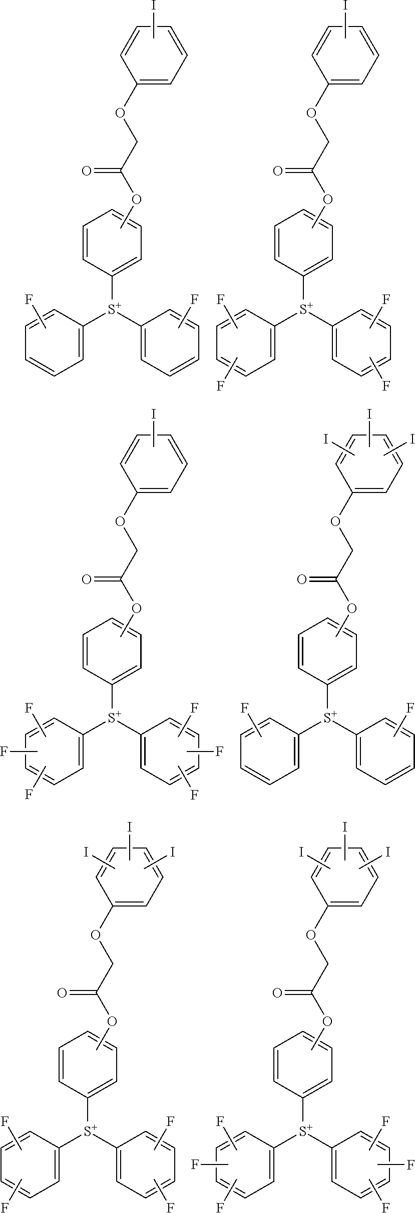

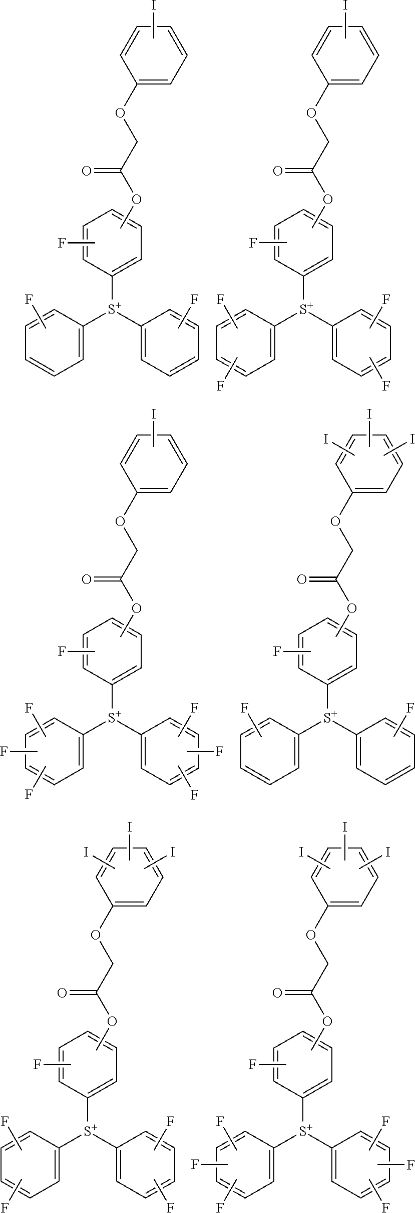

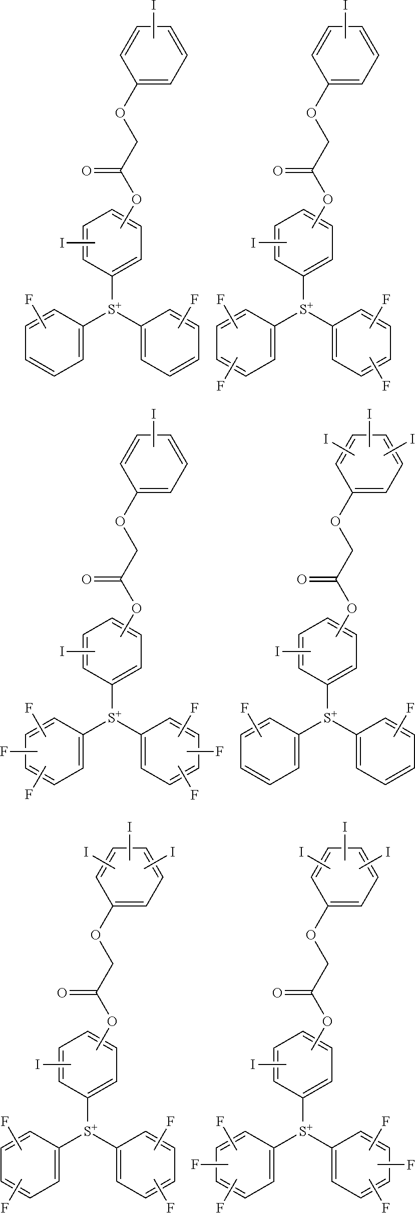

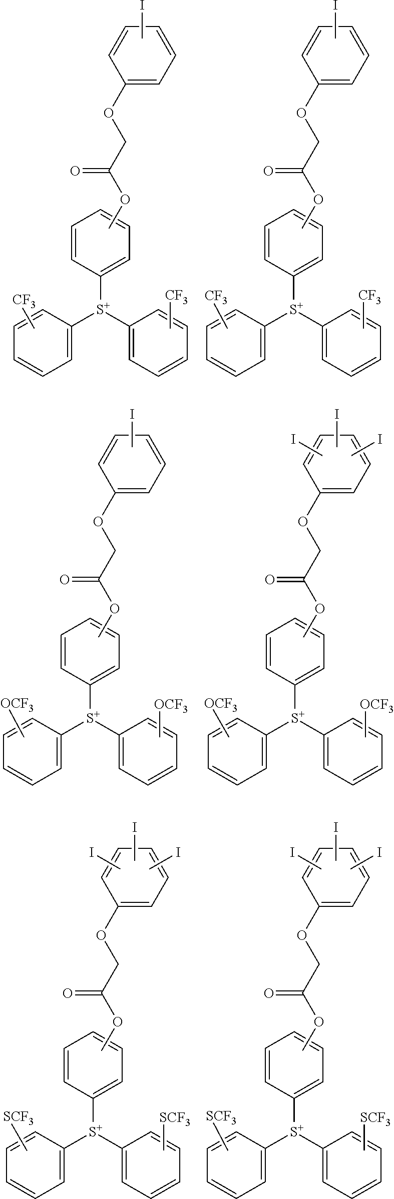

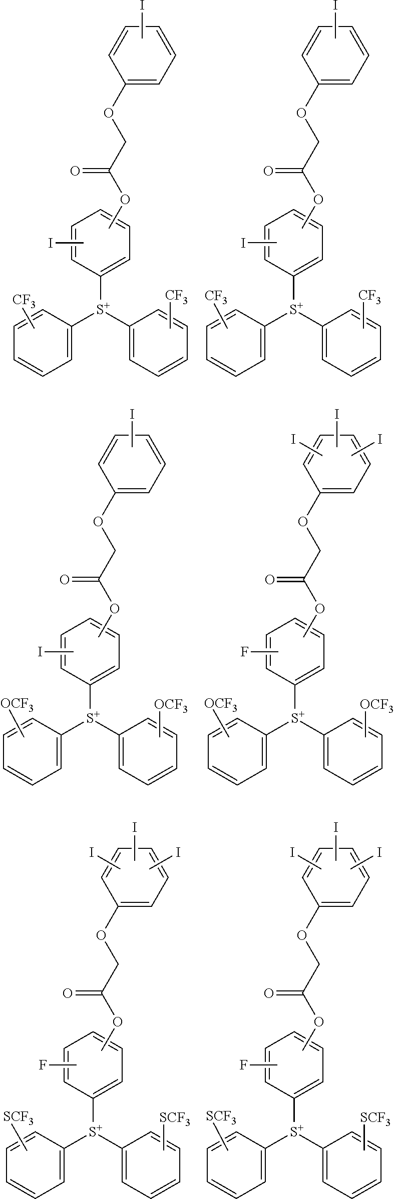

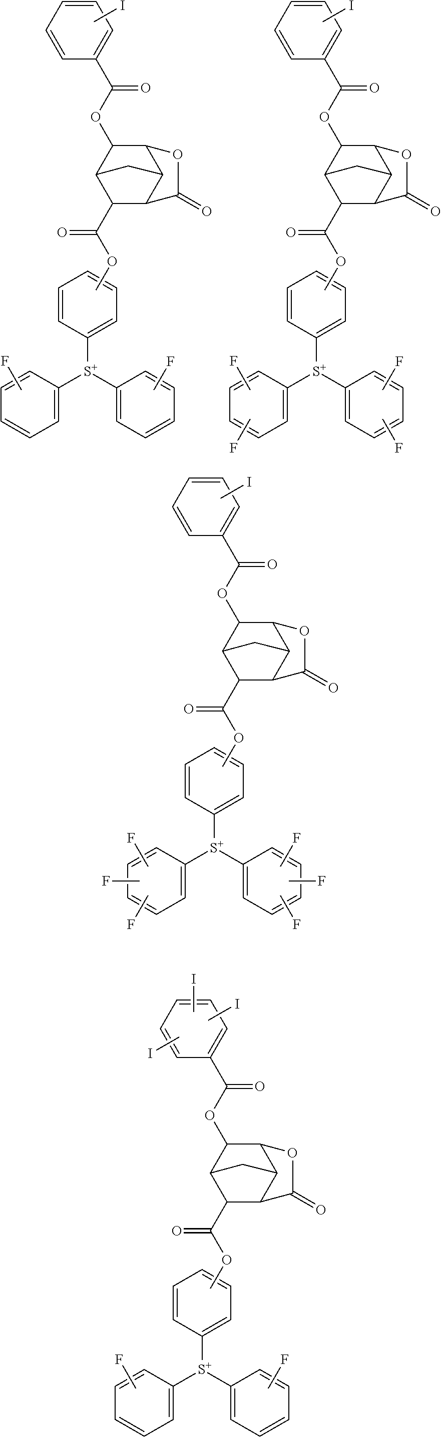

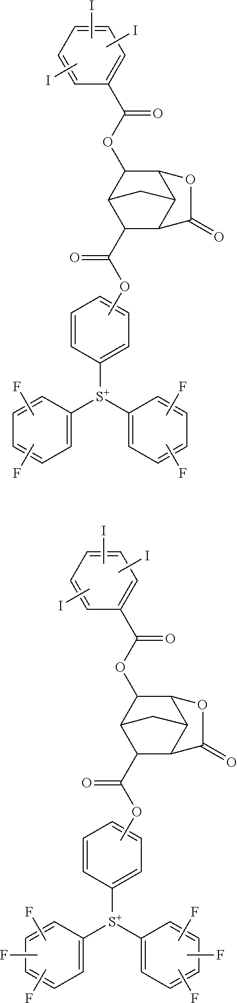

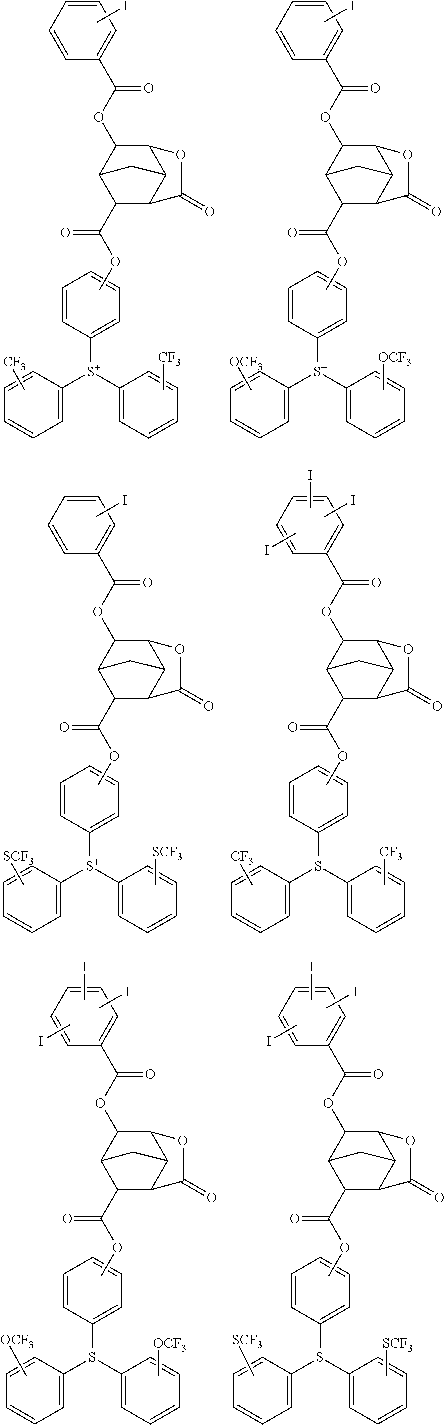

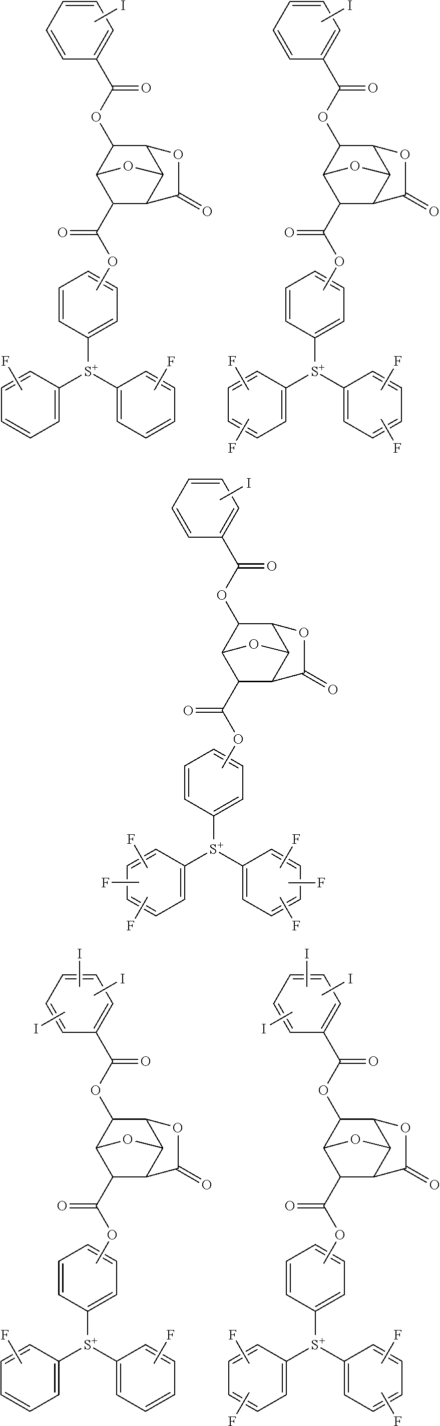

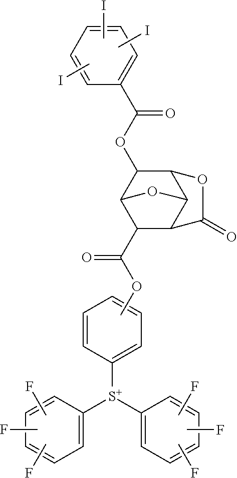

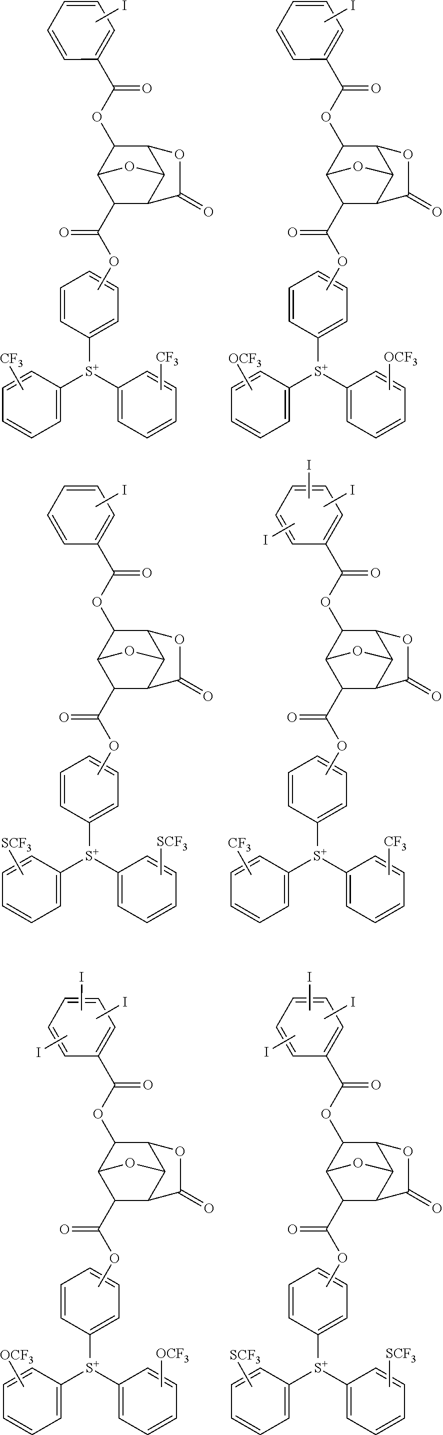

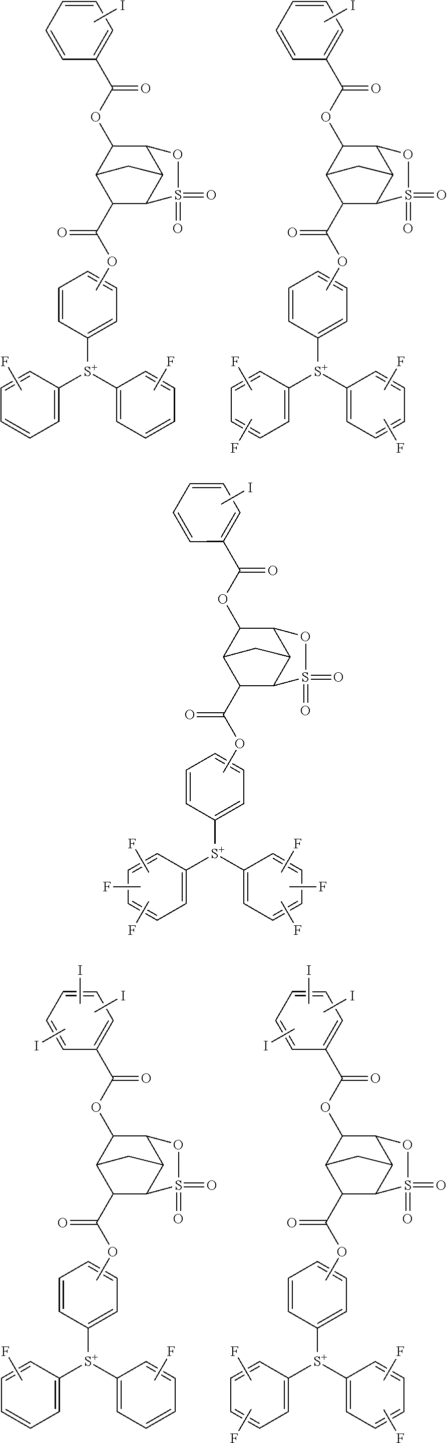

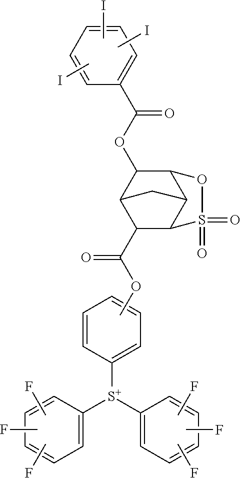

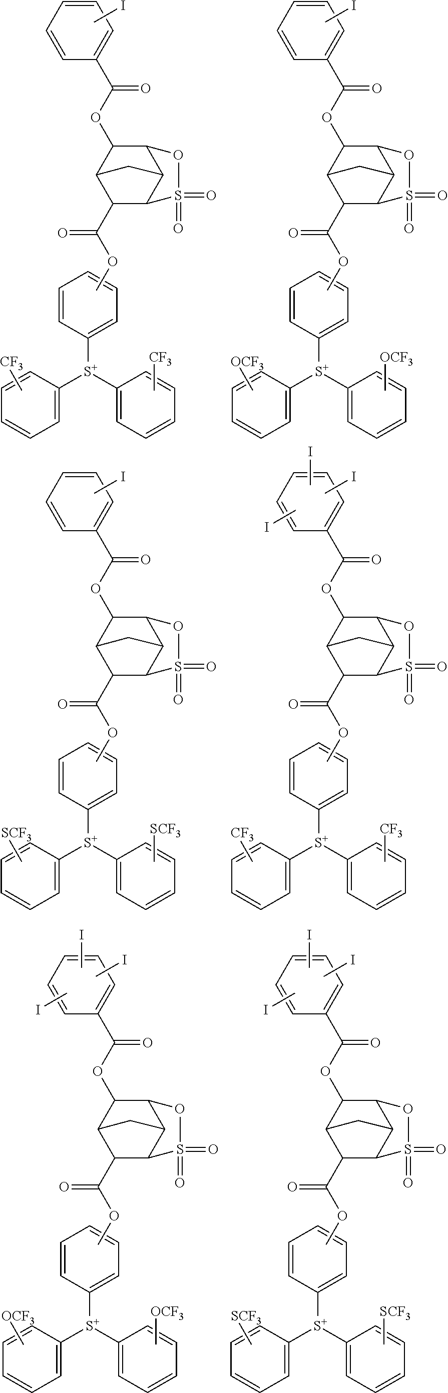

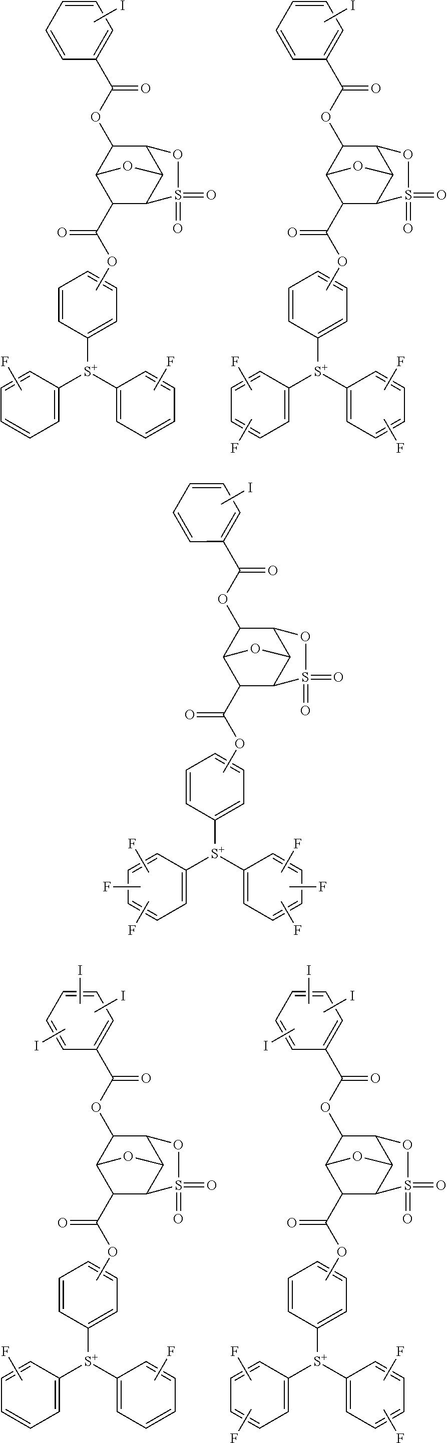

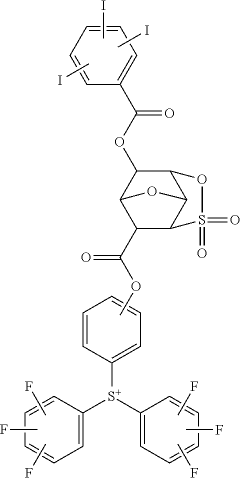

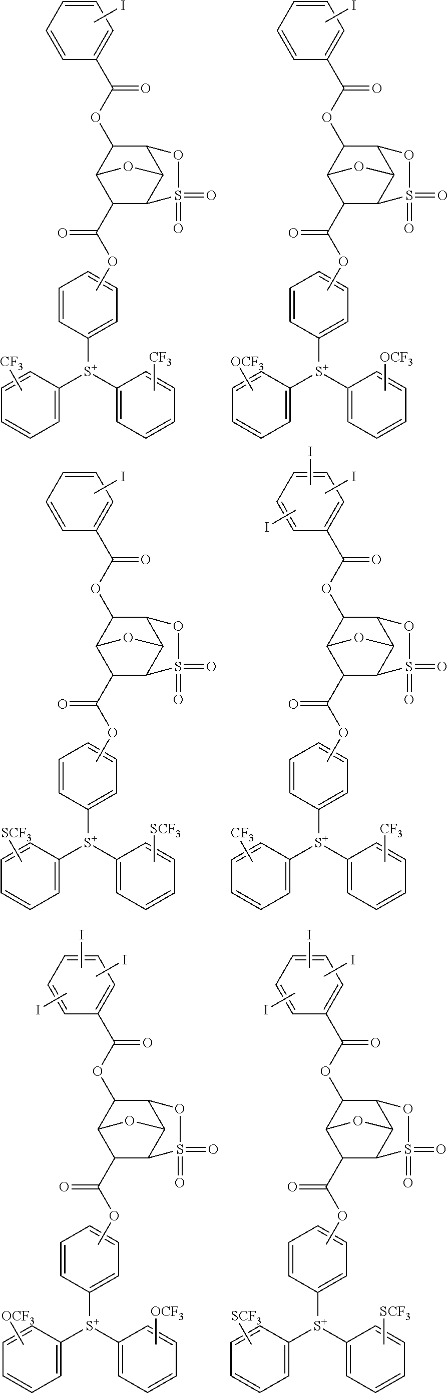

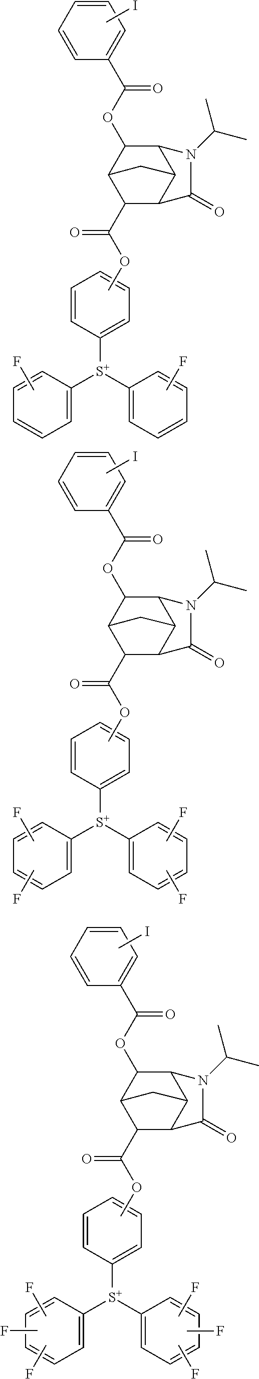

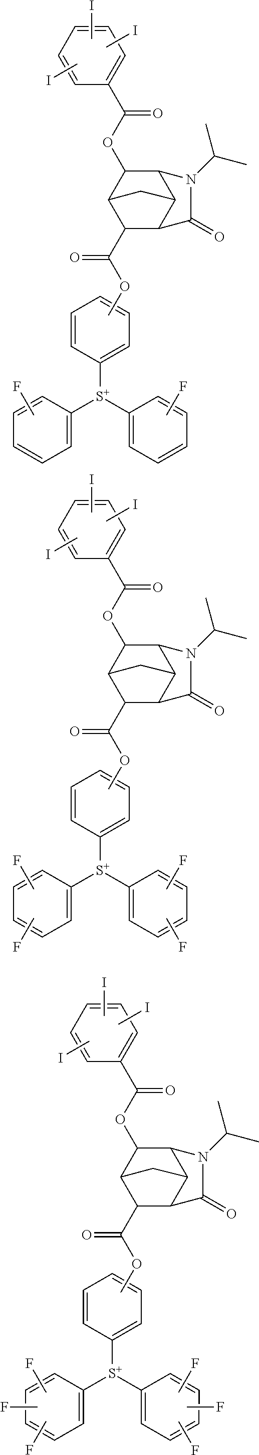

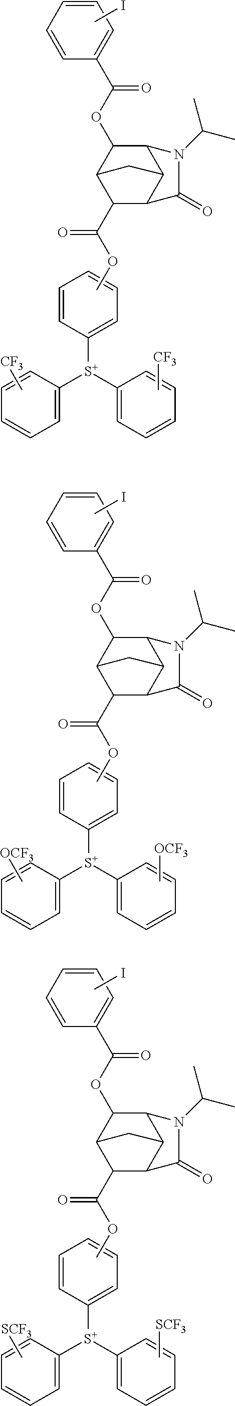

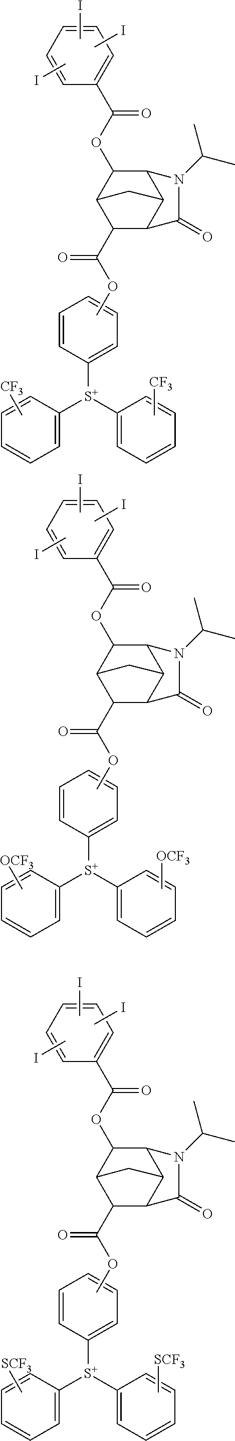

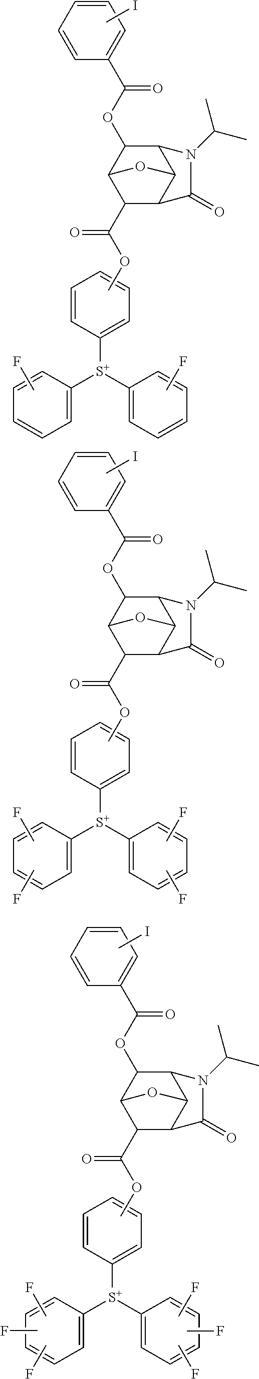

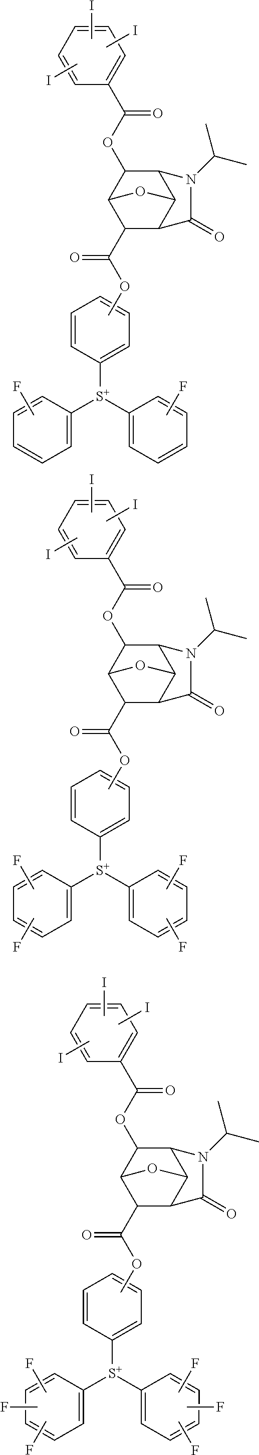

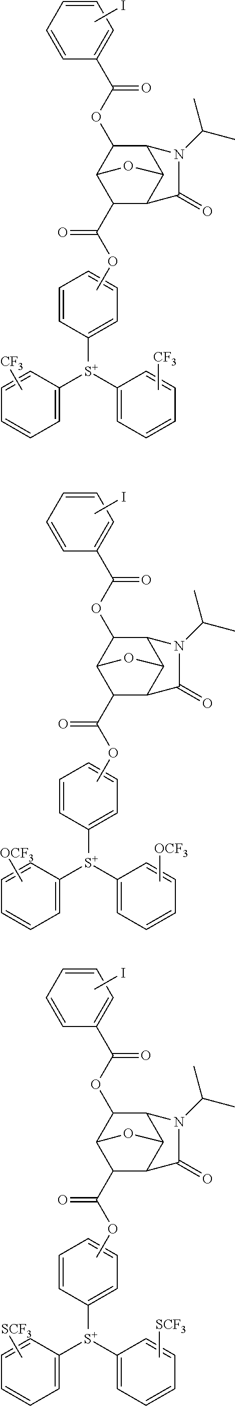

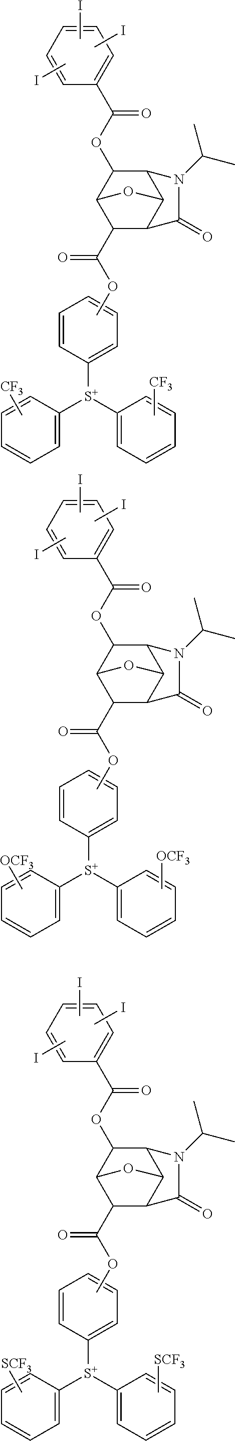

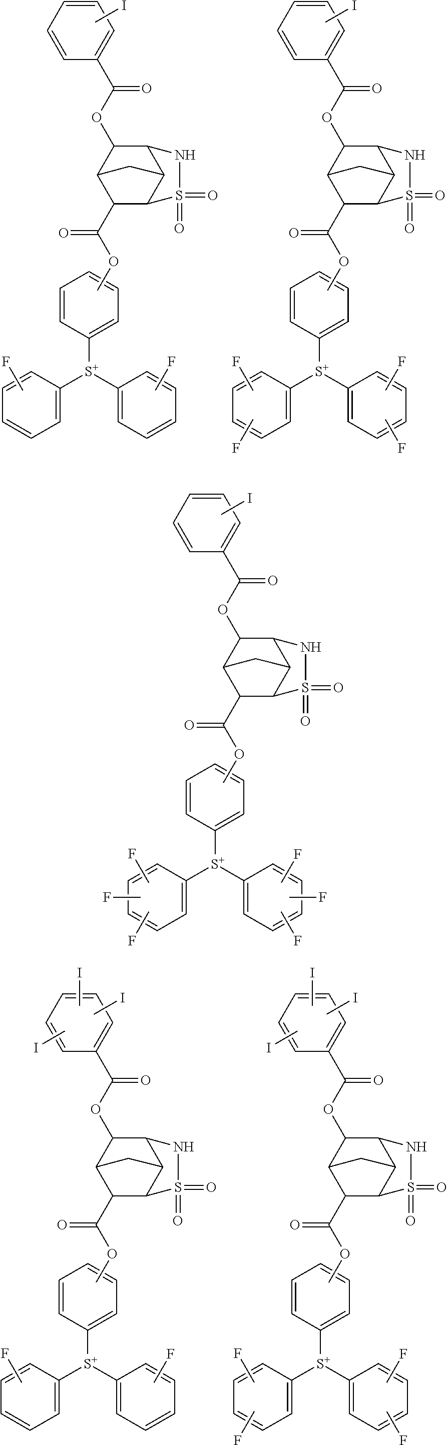

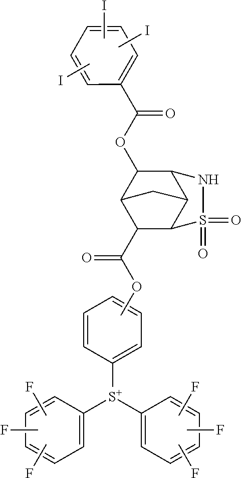

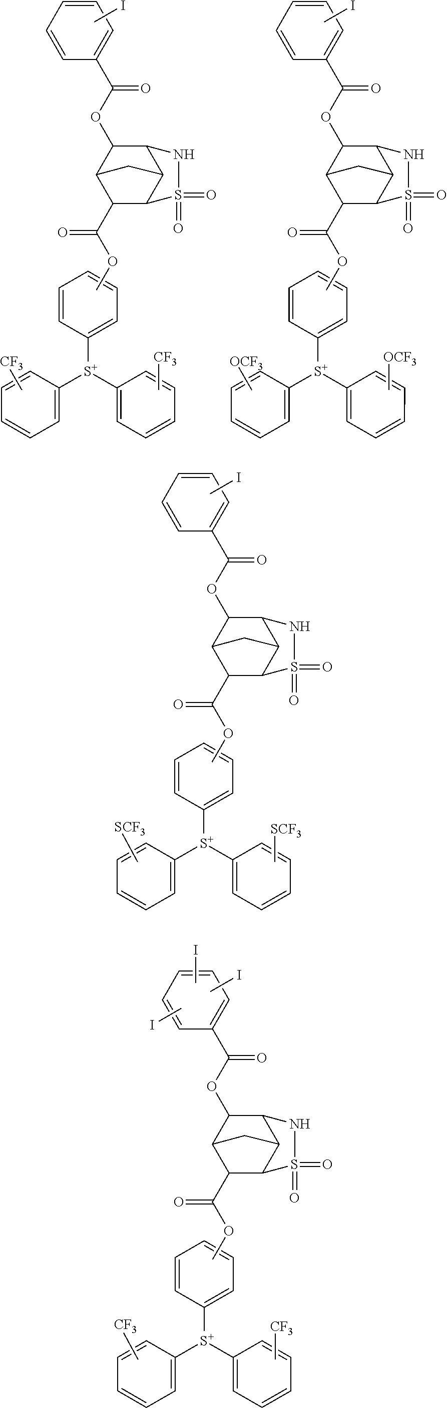

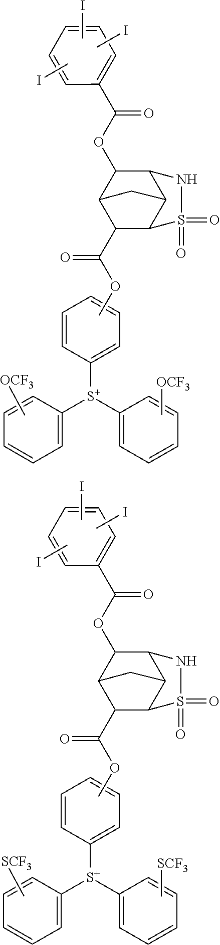

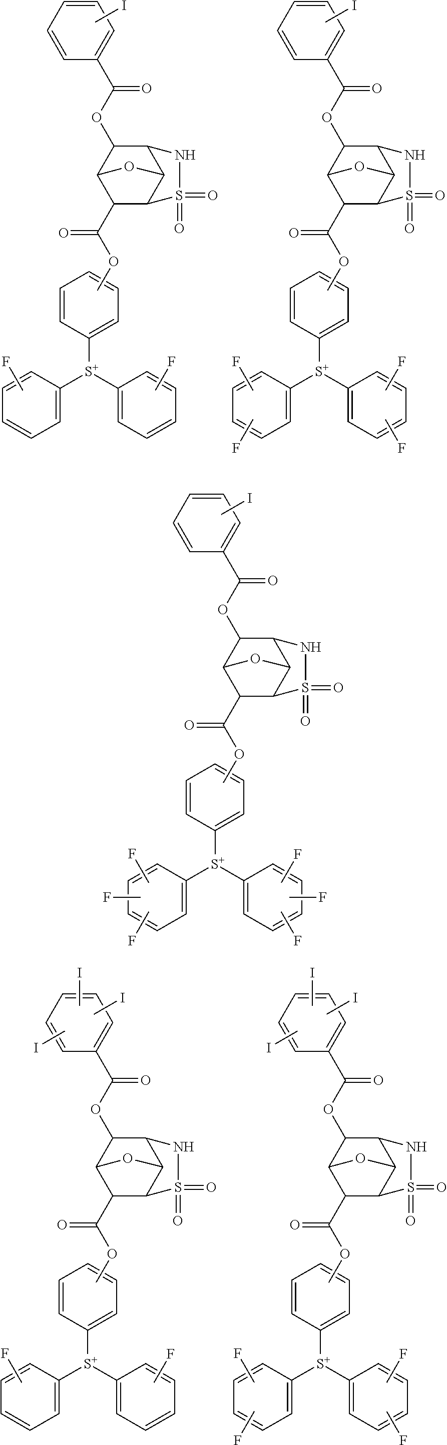

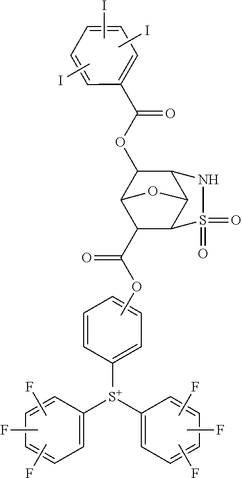

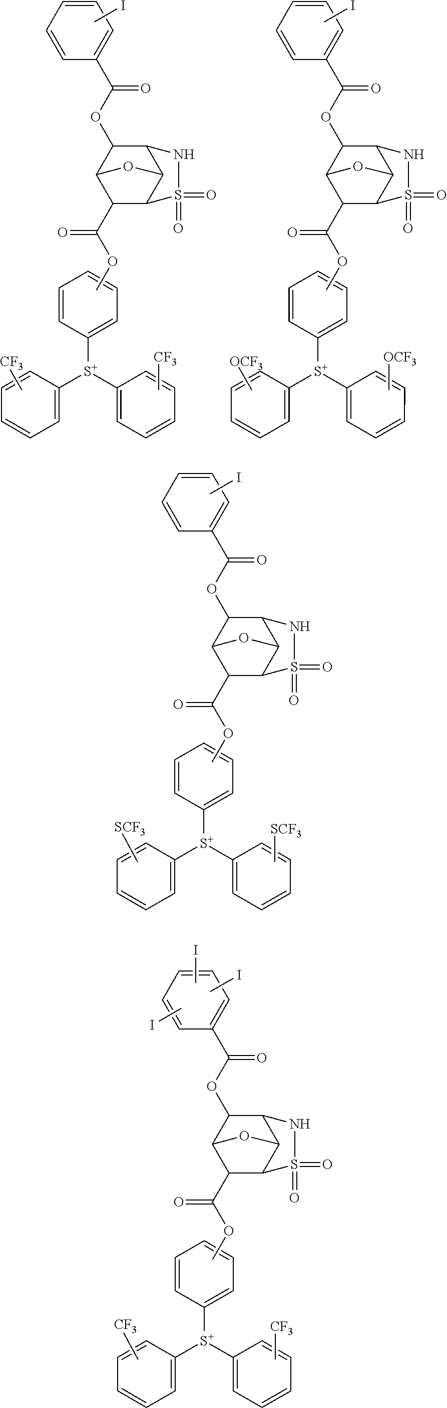

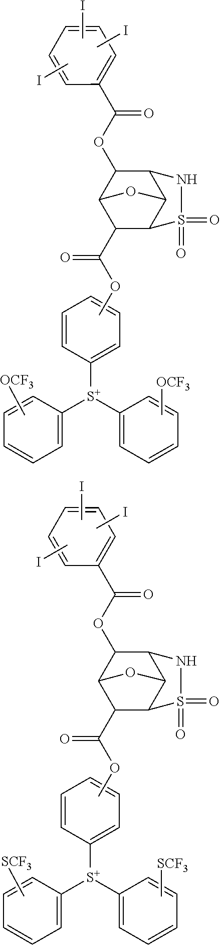

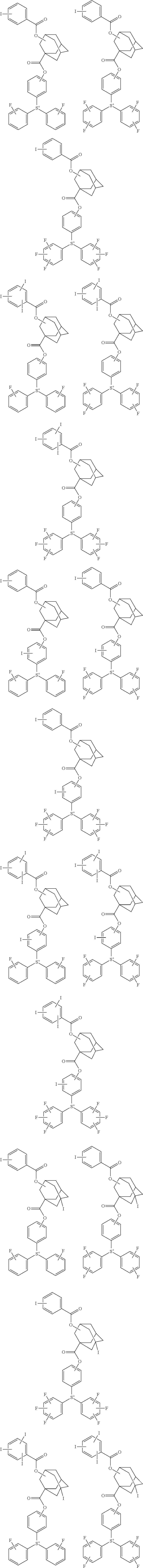

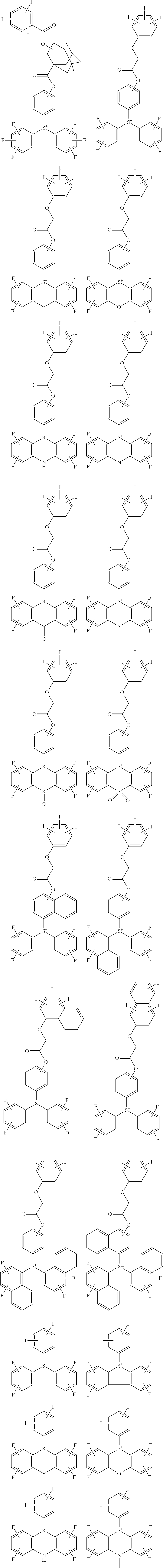

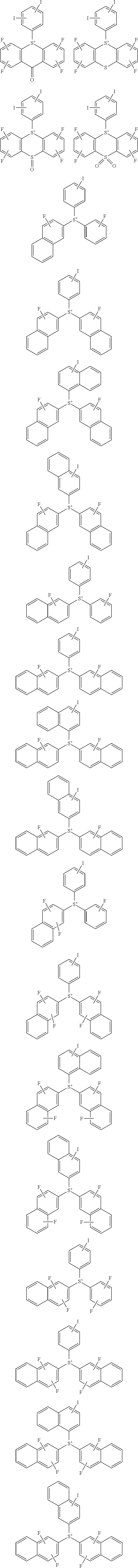

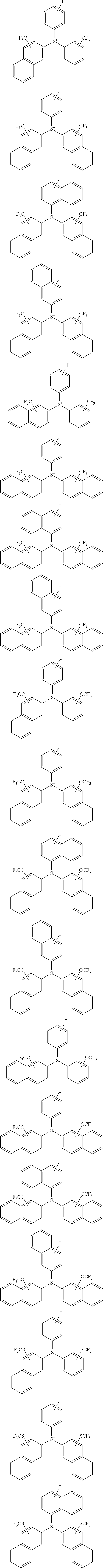

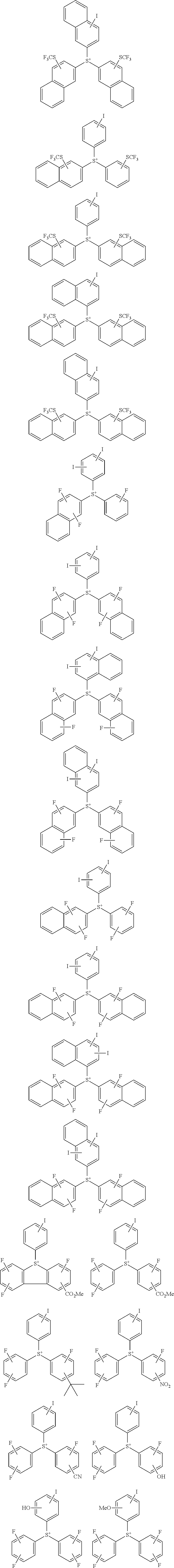

Specific examples of the onium salt type monomer of the present invention include any combination of the above-mentioned anions and cations.

The onium salt type monomer of the present invention can be synthesized by a known method. As an example, a method for manufacturing an onium salt type monomer represented by the following general formula (PAG-A-ex) will be described, but the synthesis method is not limited thereto.

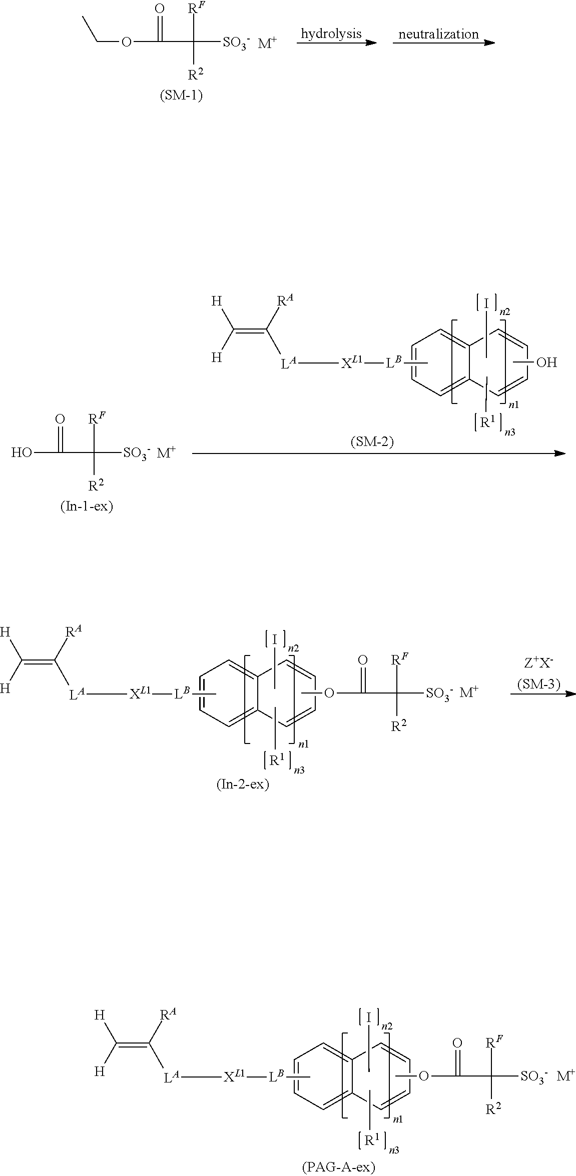

In the formulae, R^{A}, R¹, R², R^{F}, L^{A}, L^{B}, X^{L1}, “n1” to “n3”, and Z⁺ are as defined above; M⁺ is a counter cation; and X⁻ is a counter anion.

The first step is to obtain the intermediate In-1-ex through the hydrolysis and then neutralization reactions of the ester moiety of the raw material SM-1, which is commercially available or can obtained by a known synthetic method. The hydrolysis of the ester of the raw material SM-1 is preferably carried out under basic conditions, and the base used is preferably an alkali metal hydroxide such as sodium hydroxide or potassium hydroxide. The raw material SM-1 is dissolved in a solvent such as tetrahydrofuran or 1,4-dioxane, and a base is added to carry out the reaction. To ensure that the reaction proceeds smoothly, the reaction can be carried out with heating, etc., as necessary. The reaction time is usually about 4 to 12 hours, but from the viewpoint of yield, it is desirable to complete the reaction by monitoring it with silica gel thin layer chromatography (TLC). After checking the progress of the reaction, add a small excess of acid over the base used, to carry out a neutralization reaction. The acid used is preferably a strong acid, specific examples of which include hydrochloric acid, sulfuric acid, and nitric acid. After the neutralization reaction, the solvent is concentrated to obtain the intermediate In-1-ex, which can be purified by conventional methods such as chromatography and recrystallization, if necessary.

The second step is to obtain the intermediate In-2-ex through the reaction of the intermediate In-1-ex with the raw material SM-2. Various condensation reagents can be used to directly form an ester bond between the carboxy group of intermediate In-1-ex and the hydroxy group of the raw material SM-2. Condensation reagents that can be used include N,N′-dicyclohexylcarbodiimide, N,N′-diisopropylcarbodiimide, 1-[3-(dimethylamino)propyl]-3-ethylcarbodiimide, and 1-ethyl-3-(3-dimethylaminopropyl)carbodiimide hydrochloride; however, from the viewpoint of ease of removal of the urea compound that is generated as a by-product after the reaction, it is preferable to use 1-ethyl-3-(3-dimethylaminopropyl)carbodiimide hydrochloride. The reaction is carried out by dissolving the intermediate In-1-ex and the raw material SM-2 in a halogen-based solvent such as methylene chloride, and adding a condensation reagent. The reaction rate can be improved by adding 4-dimethylaminopyridine as a catalyst. The reaction time is usually about 12 to 24 hours, but from the viewpoint of yield, it is desirable to complete the reaction by monitoring it with silica gel thin layer chromatography (TLC). After the reaction has stopped, the by-product urea compound can be removed by filtration or washing with water if necessary, and the reaction liquid can then be subjected to normal aqueous work-up to obtain the intermediate In-2-ex. The obtained intermediate In-2-ex can be purified, if necessary, by conventional methods such as chromatography and recrystallization.

The third step is to exchange the intermediate In-2-ex with an onium salt (raw material SM-3) represented by Z⁺X⁻ to obtain an onium salt type monomer (PAG-A-ex). In addition, as X⁻, a chloride ion, a bromide ion, an iodide ion, or methyl sulfate anion are preferable because the exchange reaction will easily proceed quantitatively. The reaction time is usually about 4 to 12 hours, but from the viewpoint of yield, it is desirable to complete the reaction by monitoring it with TLC. The onium salt type monomer (PAG-A-ex) can be obtained from the reaction mixture by normal aqueous work-up. If necessary, it can be purified by conventional methods such as chromatography and recrystallization.

In the above scheme, the ion exchange in the third step can be easily carried out by a known method; for example, JP2007-145797A can be referred to.

The above-mentioned manufacturing method is merely an example, and the manufacturing method of the onium salt type monomer of the present invention is not limited thereto.

The characteristic structural features of the onium salt type monomer of the present invention include that it has an aromatic ring having an iodine atom and an α-fluoro (or fluoroalkyl) sulfonate anion structure. Regarding iodine atoms, particularly in EUV lithography with a wavelength of 13.5 nm, the absorption of EUV by iodine atoms is very large, so secondary electrons are generated from the iodine atoms during exposure. The onium salt type monomer of the present invention has a polymerizable group in the anion moiety, and therefore the polymer of the present invention obtained by using a monomeric photo-acid generator containing this becomes an anion-bound type photo-acid generator in which the anion side is bonded to the polymer main chain. In other words, because the acid is generated by bonding to the main chain of the polymer, the diffusion of the generated acid can be suppressed. In particular, polymerizable groups consisting of styrene or vinylnaphthalene structures are also more rigid than polymerizable groups such as methacrylate esters, and they improve the glass transition temperature (Tg) of the polymer. It is believed that the aromatic rings within or between polymers interact with each other (n-n stacking effect) to arrange the polymers in a regular pattern, which provides resistance to pattern collapse in the developer even during fine pattern formation. Furthermore, the presence of aromatic rings directly linked to the main chain provides excellent etching resistance in the etching process after fine pattern formation. On the other hand, in particular, the α-fluoro (or fluoroalkyl) sulfonate anion preferably has a carbonyl group having electron-withdrawing properties at the R-position from the sulfo group, which can ensure sufficient acidity for deprotecting the acid-labile group of the acid-labile group unit. In the case of conventional α,α-difluoroalkanesulfonate anion structures, the acid strength is too strong, causing excessive deprotection reactions of acid-labile units and resulting in a deterioration of lithographic performance. The α-fluoro (or fluoroalkyl) sulfonate anion of the onium salt type monomer of the present invention generates a sulfonic acid having a weaker acidity than the above-mentioned α,α-difluoroalkanesulfonate anion, and therefore, as the structure of the acid-labile group unit to be copolymerized, it is preferable to select one that has a tertiary ester or tertiary ether structure and has a relatively low activation energy for the deprotection reaction driven by the generation of an allyl cation or benzyl cation. When an acid-labile group that generates a simple tertiary carbocation is used, it is preferably a tertiary ester or tertiary ether having a cyclic structure. In this way, by combining a unit that generates an acid with a relatively low acidity with an acid-labile unit that has a relatively low activation energy for the deprotection reaction, the deprotection reaction occurs smoothly only in the exposed areas, preventing a decrease in resolution due to blurring of acid diffusion while maintaining good sensitivity, and making it possible to improve LWR and CDU. Therefore, the polymer of the present invention is particularly suitable as a material for a chemically amplified positive resist composition.

### [Monomeric Photo-Acid Generator]

The present invention provides a monomeric photo-acid generator consisting of the above-described onium salt type monomer.

### [Polymer]

The polymer of the present invention contains a repeating unit (hereinafter also referred to as “repeating unit-A”) derived from a monomeric photo-acid generator consisting of an onium salt type monomer represented by the general formula (A). That is, the present invention provides a polymer that contains a repeating unit derived from the monomeric photo-acid generator described above.

It is preferable that the polymer further contains at least one repeating unit selected from a repeating unit represented by the following general formula (a1) (hereinafter also referred to as “repeating unit-a1”) and a repeating unit represented by the following general formula (a2) (hereinafter also referred to as “repeating unit-a2”).

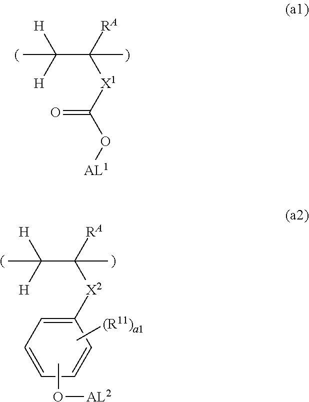

In the formulae, “a1” is an integer from 0 to 4;
R^{A} each independently represents a hydrogen atom, a fluorine atom, a methyl group, or a trifluoromethyl group;
X¹ represents a single bond, a phenylene group, a naphthylene group, or *—C(═O)—O—X¹¹—, and the phenylene group or naphthylene group may be substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom; X¹¹ represents a saturated hydrocarbylene group having 1 to 10 carbon atoms, a phenylene group, or a naphthylene group, and the saturated hydrocarbylene group may contain a hydroxy group, an ether bond, an ester bond, or a lactone ring;
X² represents a single bond or *—C(═O)—O—;
“*” represents an attachment point to a main chain carbon atom;
R²¹ represents a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; when “a1” is 2 or more, each R²¹ may be the same as or different from each other; and
AL¹ and AL² each independently represent an acid-labile group.

In the general formulae (a1) and (a2), R^{A} each independently represents a hydrogen atom, a fluorine atom, a methyl group, or a trifluoromethyl group.

In the general formula (a1), X¹ represents a single bond, a phenylene group, a naphthylene group, or *—C(═O)—O—X¹¹—, and the phenylene group or naphthylene group may be substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom. X¹¹ represents a saturated hydrocarbylene group having 1 to 10 carbon atoms, a phenylene group, or a naphthylene group, and the saturated hydrocarbylene group may contain a hydroxy group, an ether bond, an ester bond, or a lactone ring. “*” represents an attachment point to a main chain carbon atom.

In the general formula (a2), X² represents a single bond or *—C(═O)—O—. “*” represents an attachment point to a main chain carbon atom. R²¹ represents a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom. “a1” represents an integer from 0 to 4, and is preferably 0 or 1. when “a1” is 2 or more, each R²¹ may be the same as or different from each other.

In the above general formulae (a1) and (a2), AL¹ and AL² each independently represent an acid-labile group. Specific examples of the acid-labile group include those described in JP2013-080033A and JP2013-083821A.

Typical specific examples of the acid-labile group include those represented by the following general formulae (AL-1) to (AL-3).

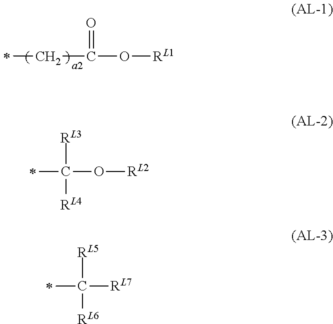

In the formulae, “*” represents an attachment point.

In the general formulae (AL-1) and (AL-2), R^{L1} and R^{L2} each independently represent a hydrocarbyl group having 1 to 40 carbon atoms, and optionally containing a heteroatom such as an oxygen atom, a sulfur atom, a nitrogen atom, or a fluorine atom. The hydrocarbyl group may be saturated or unsaturated, and may be linear, branched, or cyclic. The hydrocarbyl group preferably has 1 to 20 carbon atoms.

In the general formula (AL-1), “a2” represents an integer from 0 to 10, and preferably an integer from 1 to 5.

In the general formula (AL-2), R^{L3} and R^{L4} each independently represent a hydrogen atom or a hydrocarbyl group having 1 to 20 carbon atoms, and optionally containing a heteroatom such as an oxygen atom, a sulfur atom, a nitrogen atom, or a fluorine atom. The hydrocarbyl group may be saturated or unsaturated, and may be linear, branched, or cyclic. The hydrocarbyl group preferably has 1 to 20 carbon atoms. Furthermore, any two of R^{L2}, R^{L3}, and R^{L4} may be bonded to each other to form a ring having 3 to 20 carbon atoms together with the carbon atom or the carbon atom and oxygen atom to which they are bonded. The ring is preferably a ring having 4 to 16 carbon atoms, and particularly preferably an alicyclic ring.

In the general formula (AL-3), R^{L5}, R^{L6}, and R^{L7} each independently represent a hydrocarbyl group having 1 to 20 carbon atoms, and optionally containing a heteroatom such as an oxygen atom, a sulfur atom, a nitrogen atom, or a fluorine atom. The hydrocarbyl group may be saturated or unsaturated, and may be linear, branched, or cyclic. The hydrocarbyl group preferably has 1 to 20 carbon atoms. Furthermore, any two of R^{L5}, R^{L6}, and R^{L7} may be bonded to each other to form a ring having 3 to 20 carbon atoms together with the carbon atom to which they are bonded. The ring is preferably a ring having 4 to 16 carbon atoms, and particularly preferably an alicyclic ring.

Other structures of the acid-labile group include those described in paragraphs [0064] to [0068] of JP2023-123222A and those described in paragraphs [0013] to [0014] of JP7492842B2. These reactions are driven by the generation of conjugated olefins or acrylate derivatives after an acid elimination reaction.

Specific examples of the repeating unit-a1 include, but are not limited to, the following. In the following formulae, R^{A} and AL¹ are as defined above.

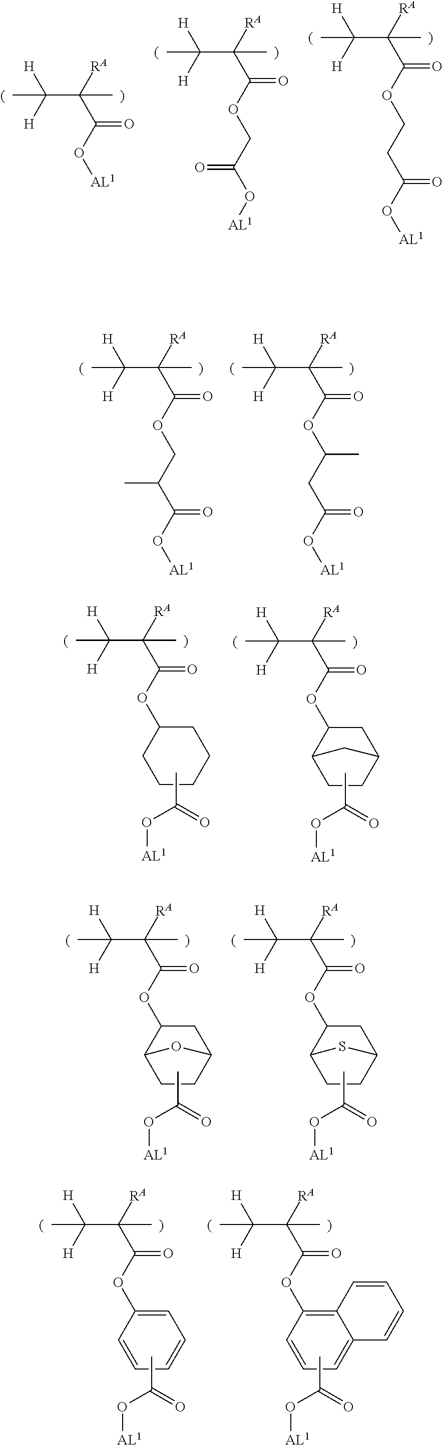

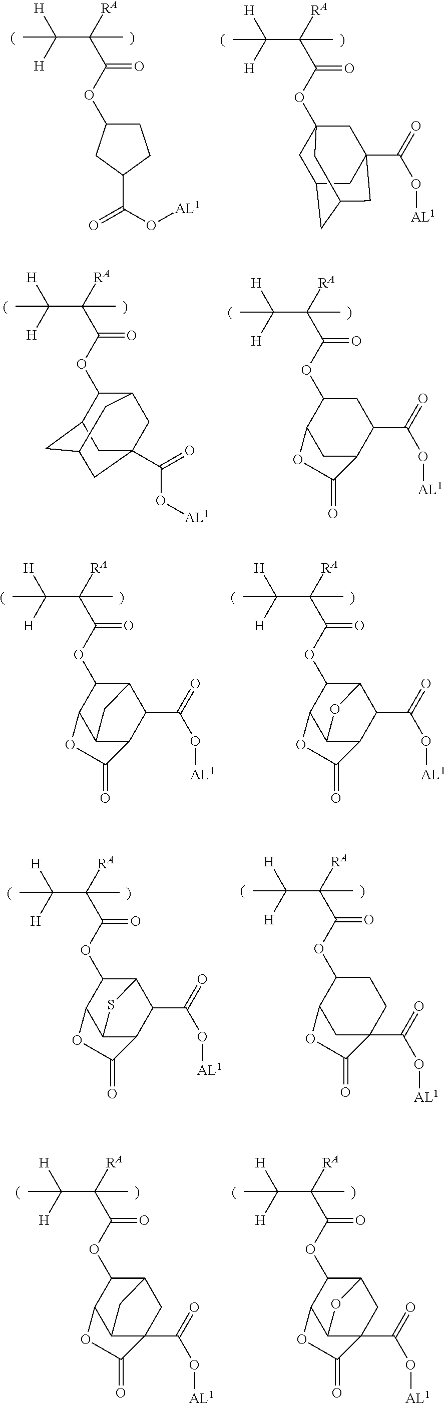

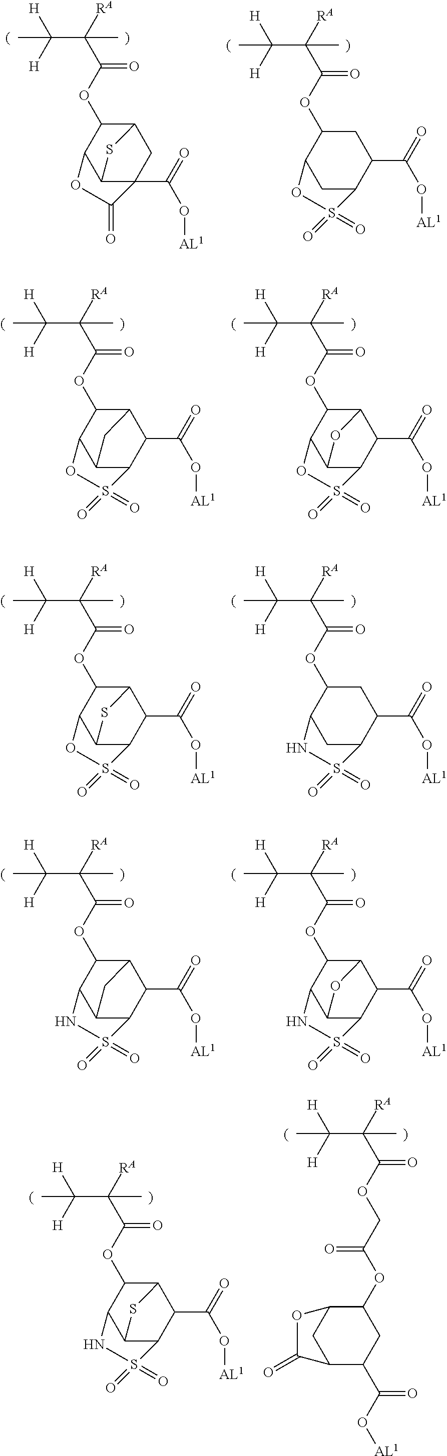

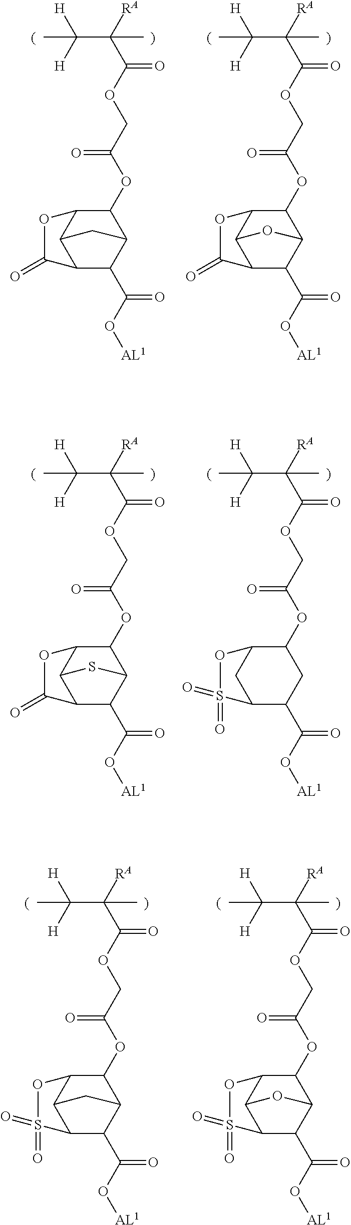

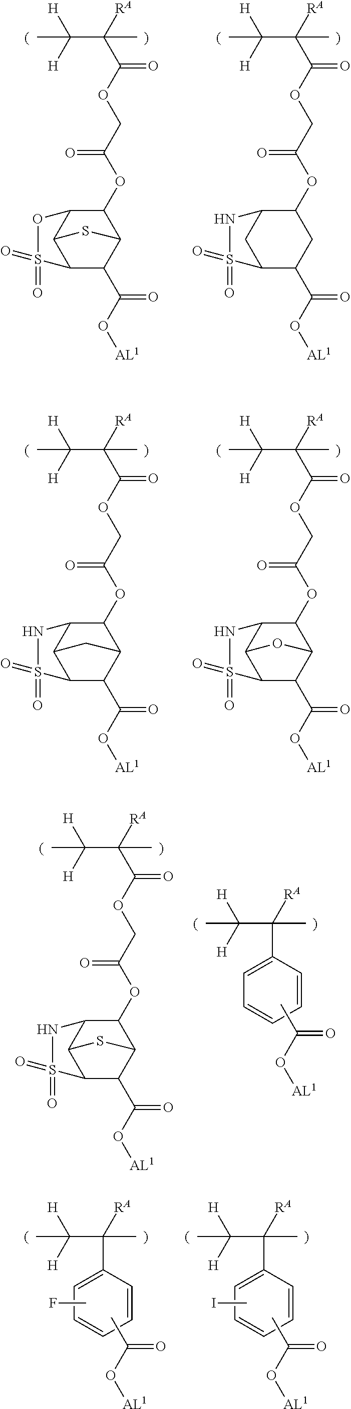

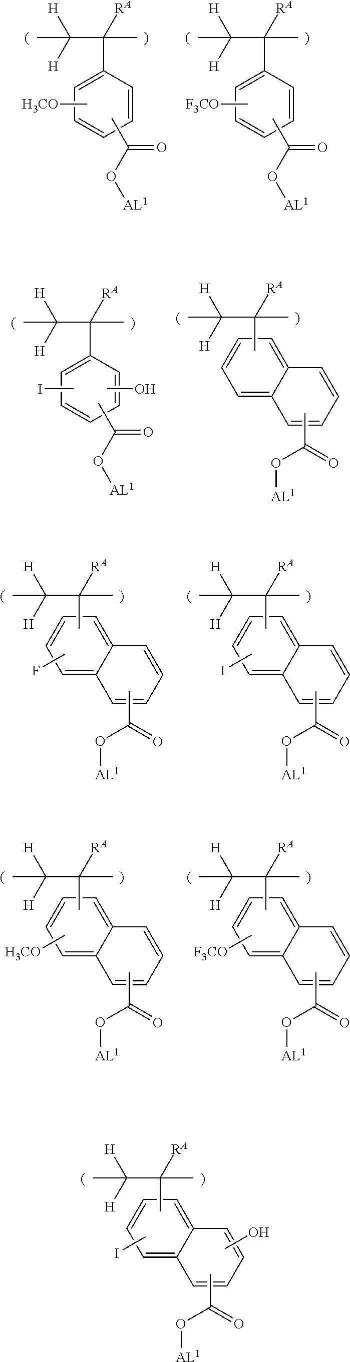

Specific examples of the repeating unit-a2 include, but are not limited to, the following. In the following formulae, R^{A} and AL² are as defined above.

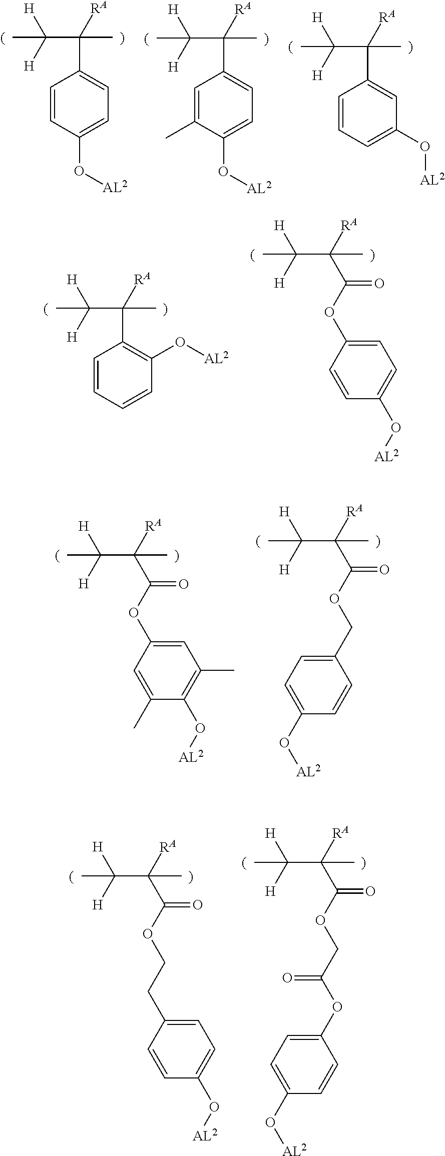

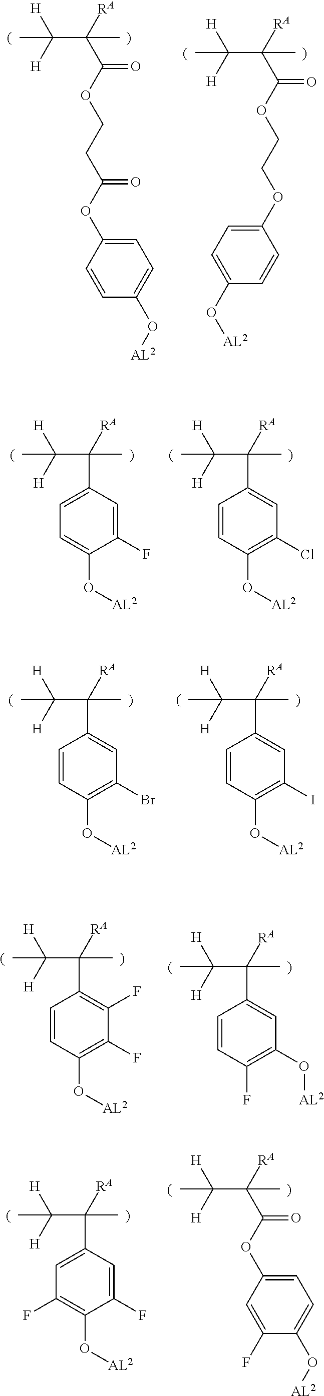

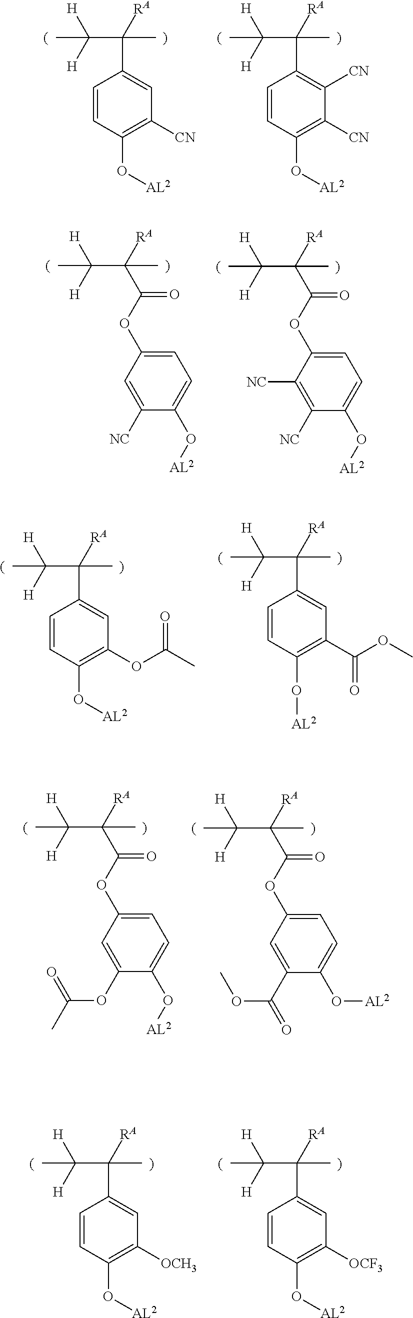

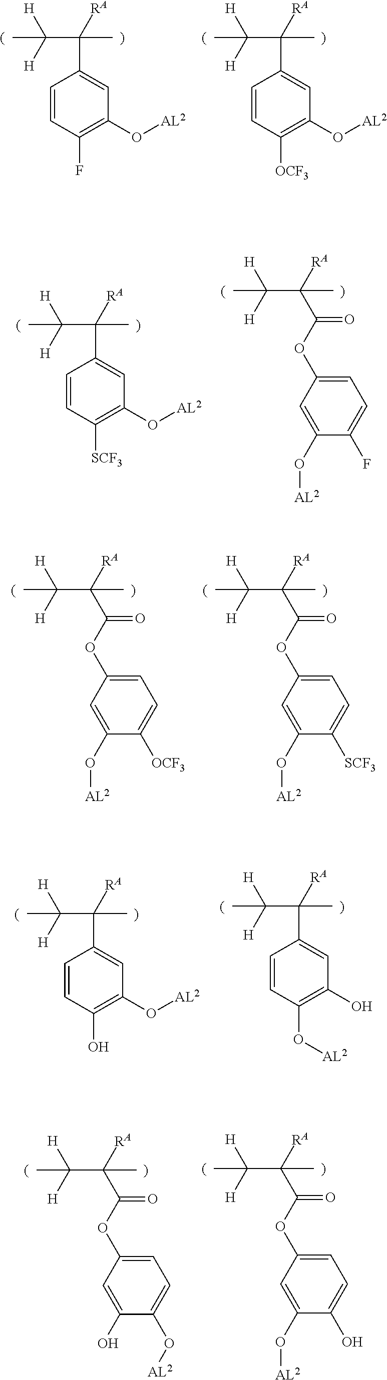

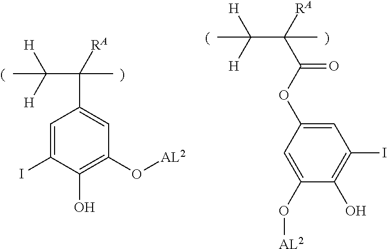

The polymer may contain a repeating unit represented by the following general formula (a3) (hereinafter also referred to as “repeating unit-a3”).

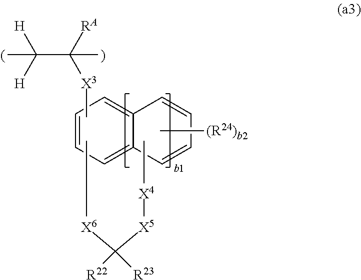

In the formula, “b1” represents 0 or 1; “b2” represents an integer from 0 to 3 when “b1” is 0, and represents an integer from 0 to 5 when “b1” is 1;
R^{A} represents a hydrogen atom, a fluorine atom, a methyl group, or a trifluoromethyl group;
X³ represents a single bond, *—C(═O)—O—, or *—C(═O)—NH—; “*” represents an attachment point to a main chain carbon atom;
X⁴ represents a single bond, an aliphatic hydrocarbylene group having 1 to 4 carbon atoms, a carbonyl group, a sulfonyl group, or a group obtained by combining these;
X⁵ and X⁶ each independently represent an oxygen atom or a sulfur atom; provided that X⁴ and X⁶ are bonded to adjacent carbon atoms of an aromatic ring;
R²² and R²³ each independently represent a hydrogen atom, or a hydrocarbyl group having 1 to 20 carbon atoms and optionally containing a heteroatom; also, R²² and R²³ may be bonded to each other to form a ring together with the carbon atom to which they are bonded;
R²⁴ represents a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, a hydrocarbylthio group having 1 to 20 carbon atoms and optionally containing a heteroatom, or —N(R^{24A}) (R^{24B}); R^{24A} and R^{24B} each independently represent a hydrogen atom or a hydrocarbyl group having 1 to 6 carbon atoms; and when “b2” is 2 or more, each R²⁴ may be the same as or different from each other, and a plurality of R²⁴ may be bonded to each other to form a ring together with the carbon atoms of the aromatic ring to which they are bonded.

In the general formula (a3), “b1” represents 0 or 1. When “b1” is 0, the structure is a benzene ring, and when “b1” is 1, it is a naphthalene ring; however, from the viewpoint of solvent solubility, it is preferable that “b1” is 0, giving a benzene ring. “b2” represents an integer from 0 to 3 when “b1” is 0, and represents an integer from 0 to 5 when “b1” is 1. From the viewpoint of raw material procurement, “b2” is preferably 0, 1, 2, or 3, and more preferably 0, 1, or 2.

In the general formula (a3), R^{A} represents a hydrogen atom, a fluorine atom, a methyl group, or a trifluoromethyl group. Among these, a hydrogen atom or a methyl group is preferable, and a hydrogen atom is further preferable.

In the general formula (a3), X³ is a single bond, *—C(═O)—O—, or *—C(═O)—NH—. “*” represents an attachment point to a main chain carbon atom. Among these, a single bond, or —C(═O)—O— is preferable, and a single bond is further preferable.

In the general formula (a3), X⁴ represents a single bond, an aliphatic hydrocarbylene group having 1 to 4 carbon atoms, a carbonyl group, a sulfonyl group, or a group obtained by combining these. Among these, from the viewpoint of raw material procurement, a single bond, a carbonyl group, or a sulfonyl group is preferable, and from the viewpoint of a polar group generated after the reaction, a single bond, or a carbonyl group is more preferable.

In the general formula (a3), X⁵ and X⁶ each independently represent an oxygen atom or a sulfur atom. However, X⁴ and X⁶ are bonded to adjacent carbon atoms of an aromatic ring. X⁵ and X⁶ may be the same as or different from each other, but from the viewpoint of reactivity, both X⁵ and X⁶ are preferably oxygen atoms.

In the general formula (a3), R²² and R²³ each independently represent a hydrogen atom, or a hydrocarbyl group having 1 to 20 carbon atoms and optionally containing a heteroatom. The hydrocarbyl group may be saturated or unsaturated, and may be linear, branched, or cyclic. Specific examples thereof include alkyl groups having 1 to 20 carbon atoms, such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl groups; cyclic saturated hydrocarbyl groups having 3 to 20 carbon atoms, such as cyclopropyl, cyclopentyl, cyclohexyl, cyclopropylmethyl, 4-methylcyclohexyl, cyclohexylmethyl, norbornyl, and adamantyl groups; alkenyl groups having 2 to 20 carbon atoms, such as vinyl, 1-propenyl, 2-propenyl, butenyl, and hexenyl groups; cyclic unsaturated hydrocarbyl groups having 3 to 20 carbon atoms, such as cyclohexenyl groups; aryl groups having 6 to 20 carbon atoms, such as phenyl and naphthyl groups; aralkyl groups having 7 to 20 carbon atoms, such as benzyl, 1-phenylethyl, and 2-phenylethyl groups; and groups obtained by combining these groups. Furthermore, some or all of the hydrogen atoms of the hydrocarbyl groups may be substituted with a group containing a heteroatom such as an oxygen atom, a sulfur atom, a nitrogen atom, or a halogen atom, and some of the —CH₂— of the hydrocarbyl groups may be substituted with a group containing a heteroatom such as an oxygen atom, a sulfur atom, or a nitrogen atom, and as a result, they may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group.

Furthermore, R²² and R²³ may be bonded to each other to form a ring together with the carbon atom to which they are bonded. Specific examples of the ring formed in this case include a cyclopropane ring, a cyclobutane ring, a cyclopentane ring, a cyclohexane ring, a norbornane ring, and an adamantane ring. Furthermore, some or all of the hydrogen atoms in the ring may be substituted with a group containing a heteroatom such as an oxygen atom, a sulfur atom, a nitrogen atom, or a halogen atom, and some of the —CH₂— in the ring may be substituted with a group containing a heteroatom such as an oxygen atom, a sulfur atom, or a nitrogen atom, and as a result, it may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group.

In the general formula (a3), R²⁴ represents a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, a hydrocarbylthio group having 1 to 20 carbon atoms and optionally containing a heteroatom, or —N(R^{24A}) (R^{24B}) R^{24A} and R^{24B} each independently represent a hydrogen atom or a hydrocarbyl group having 1 to 6 carbon atoms. The halogen atom is preferably a fluorine atom, a chlorine atom, a bromine atom, or an iodine atom, and more preferably a fluorine atom or an iodine atom. The hydrocarbyl groups and the hydrocarbyl moieties of the hydrocarbyloxy, hydrocarbyloxycarbonyl, and hydrocarbylthio groups may be saturated or unsaturated, and may be linear, branched, or cyclic. Specific examples thereof include the same ones as those given as examples of the hydrocarbyl group represented by R²² and R²³. Furthermore, some or all of the hydrogen atoms of the hydrocarbyl groups may be substituted with a group containing a heteroatom such as an oxygen atom, a sulfur atom, a nitrogen atom, or a halogen atom, and some of the —CH₂— of the hydrocarbyl groups may be substituted with a group containing a heteroatom such as an oxygen atom, a sulfur atom, or a nitrogen atom, and as a result, they may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group. When “b2” is 2 or more, each R²⁴ may be the same as or different from each other.

Furthermore, when “b2” is 2 or more, a plurality of R²⁴ may be bonded to each other to form a ring together with the carbon atoms of the aromatic ring to which they are bonded. Specific examples of the ring formed in this case include a cyclopropane ring, a cyclobutane ring, a cyclopentane ring, a cyclohexane ring, a norbornane ring, and an adamantane ring. Furthermore, some or all of the hydrogen atoms in the ring may be substituted with a group containing a heteroatom such as an oxygen atom, a sulfur atom, a nitrogen atom, or a halogen atom, and some of the —CH₂— in the ring may be substituted with a group containing a heteroatom such as an oxygen atom, a sulfur atom, or a nitrogen atom, and as a result, it may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group.

That is, it is preferable that the polymer further contains at least one repeating unit selected from the group consisting of a repeating unit represented by the general formula (a1), a repeating unit represented by the general formula (a2), and a repeating unit represented by the general formula (a3).

Specific examples of the repeating unit-a3 include, but are not limited to, the following. In the following formulae, R^{A} is as defined above, and Me represents a methyl group. Furthermore, the bonding positions of the various substituents on the aromatic ring may be interchanged.

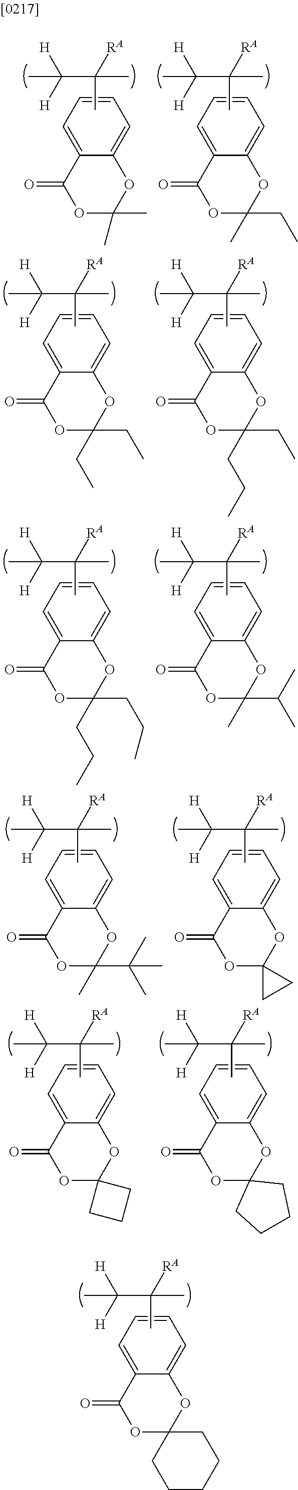

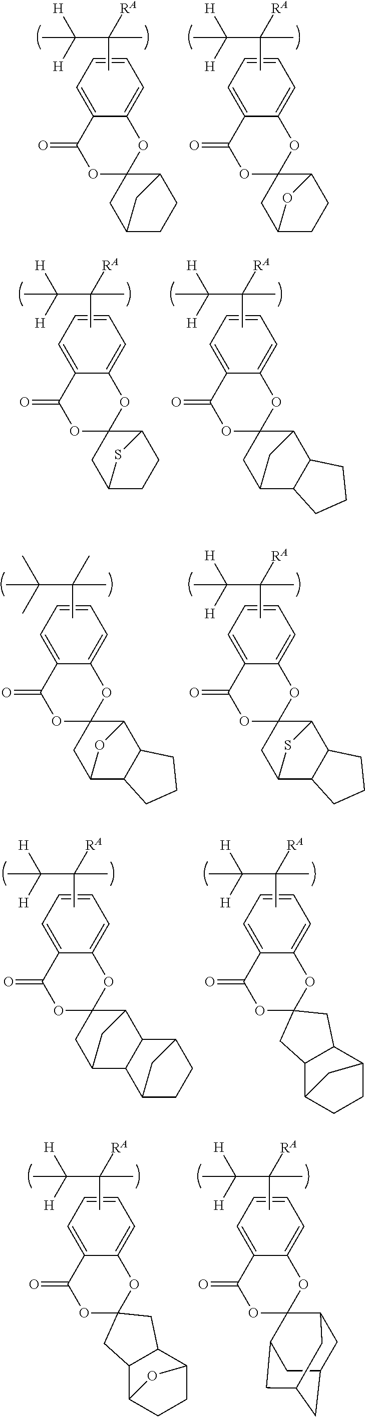

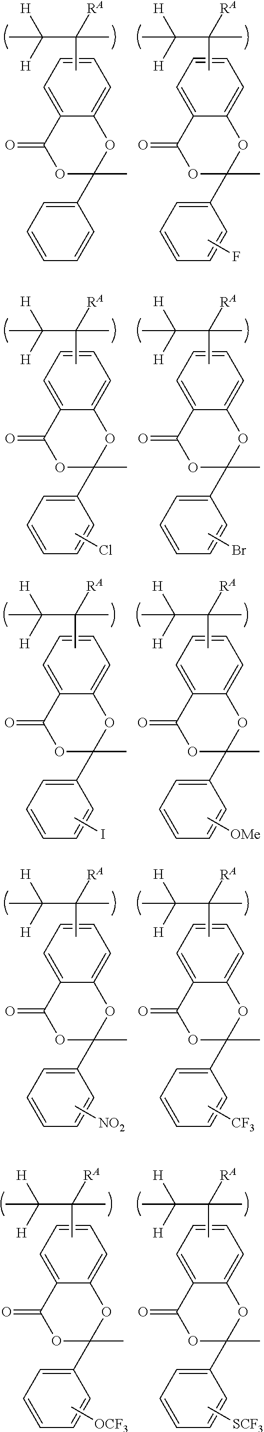

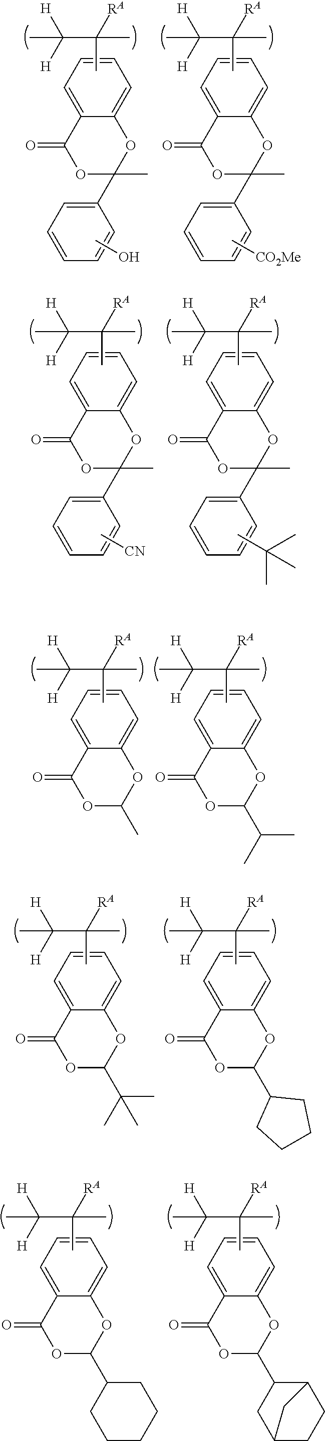

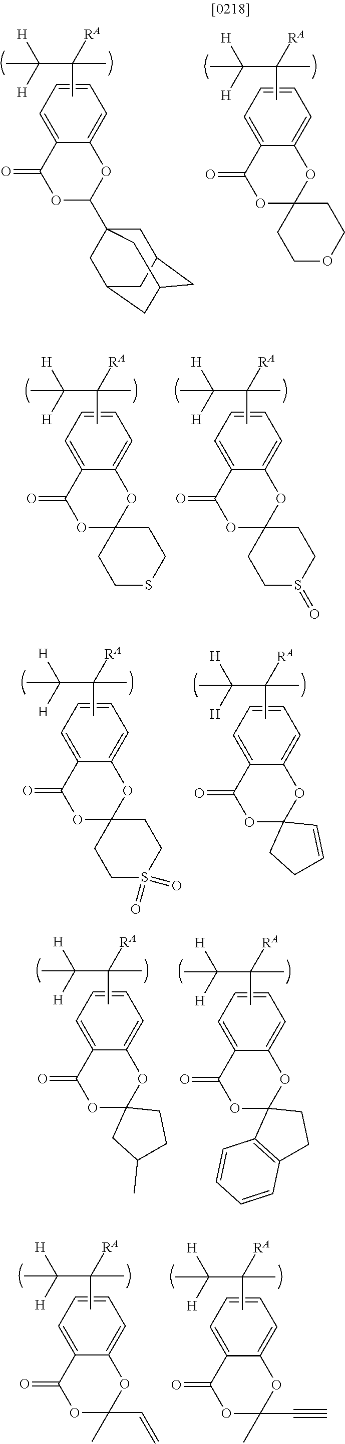

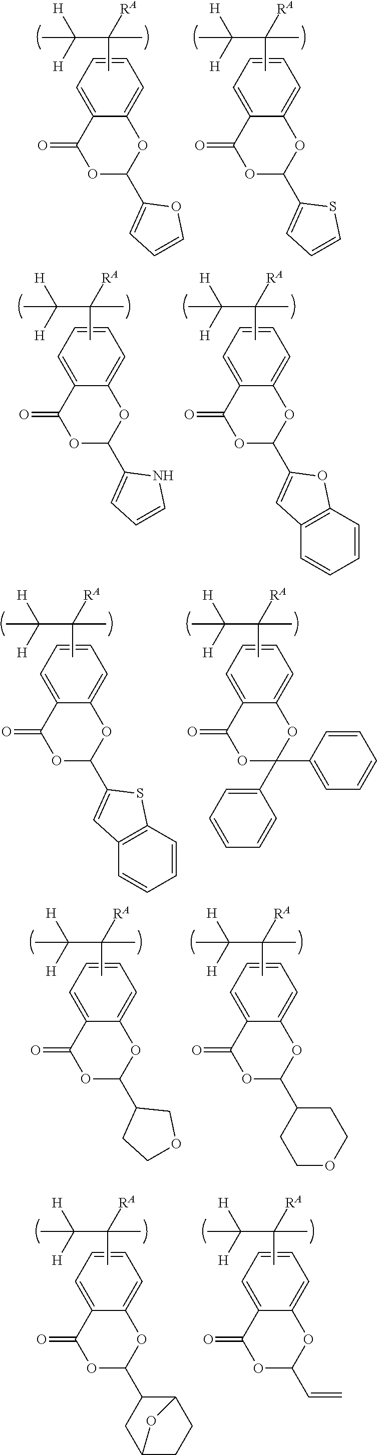

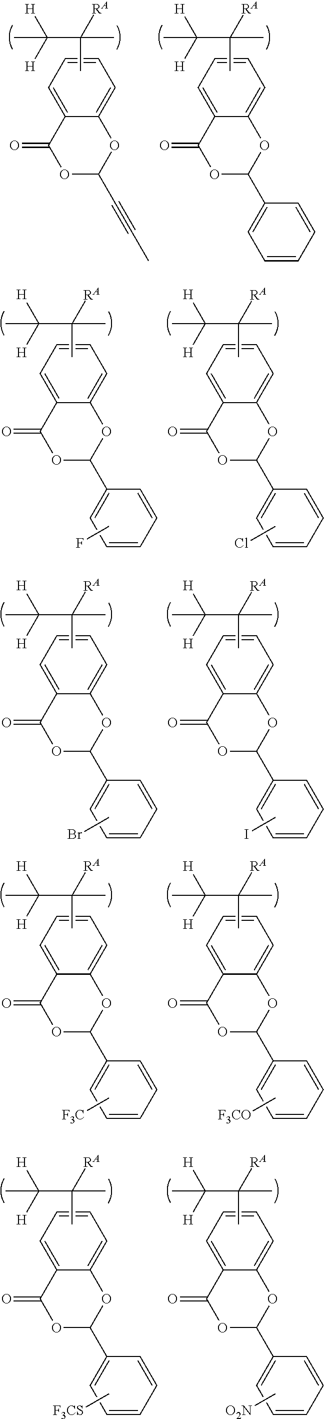

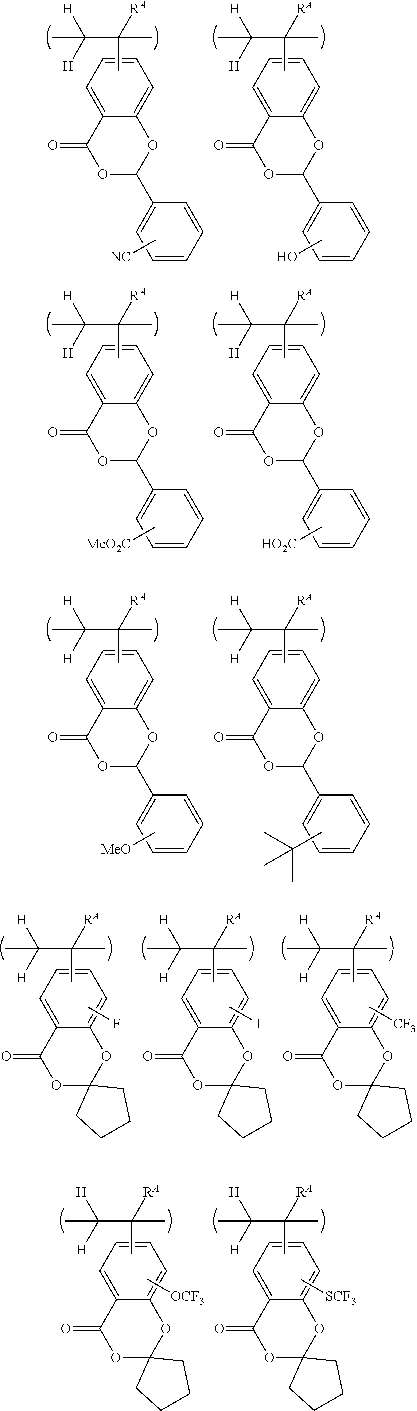

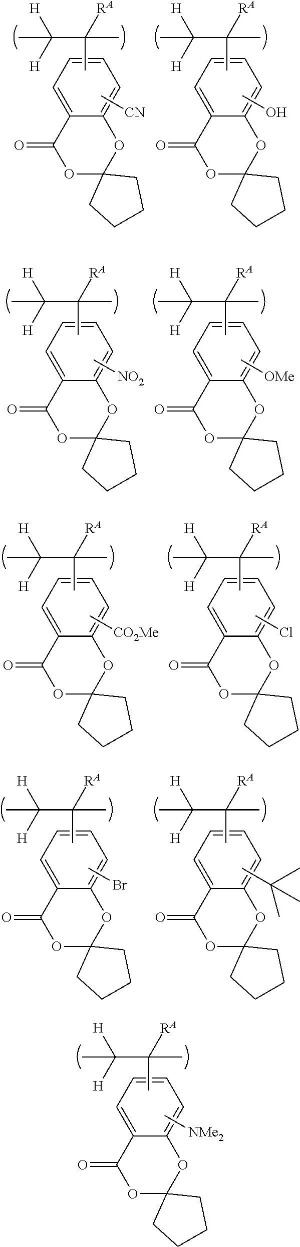

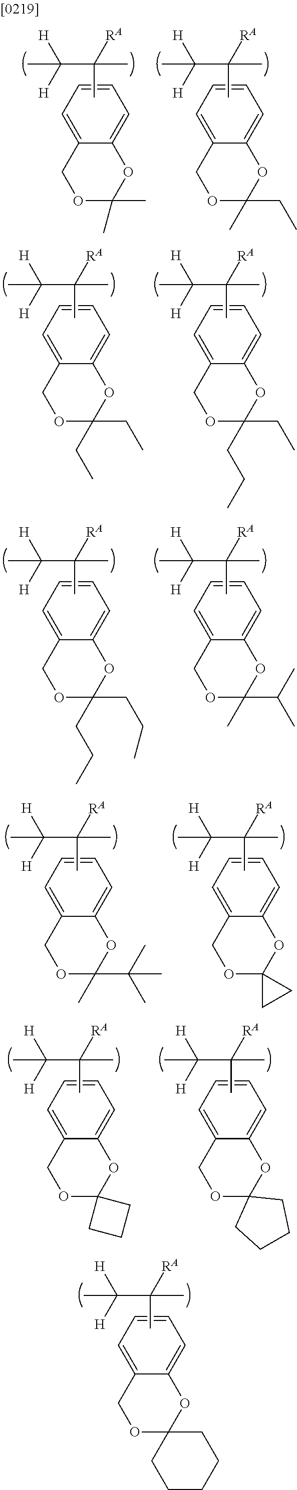

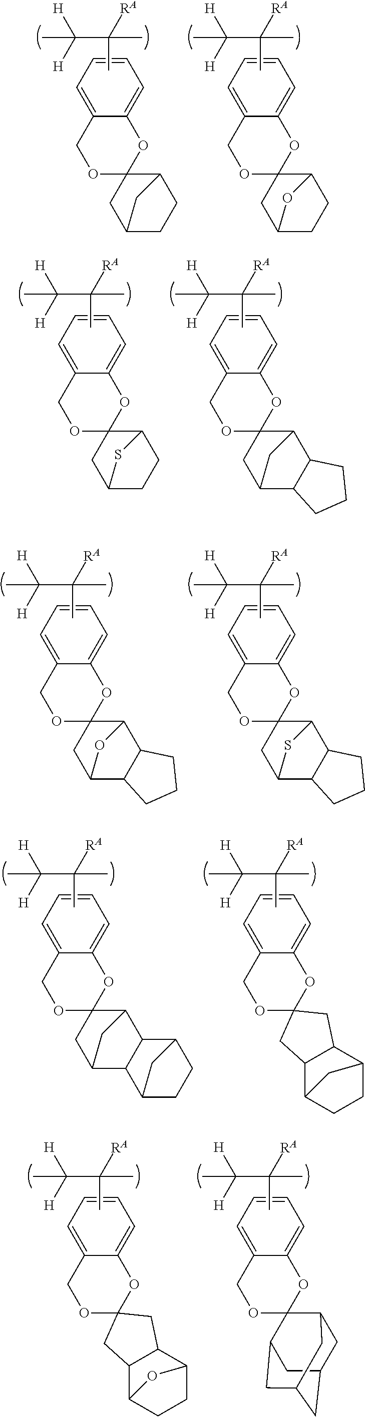

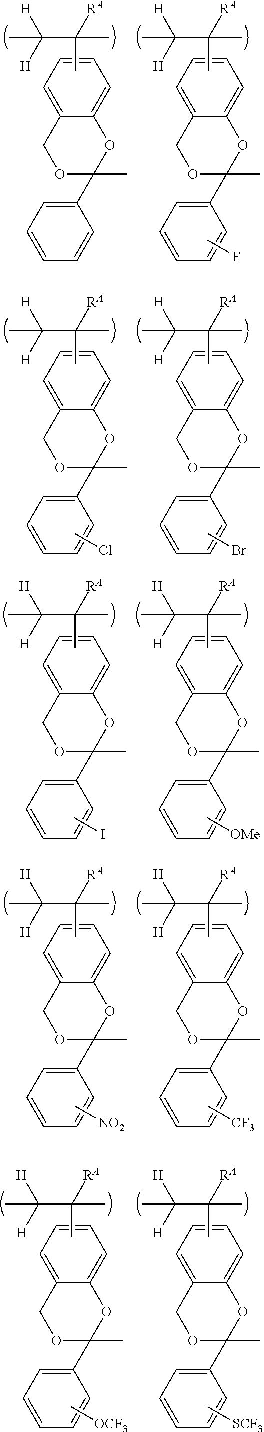

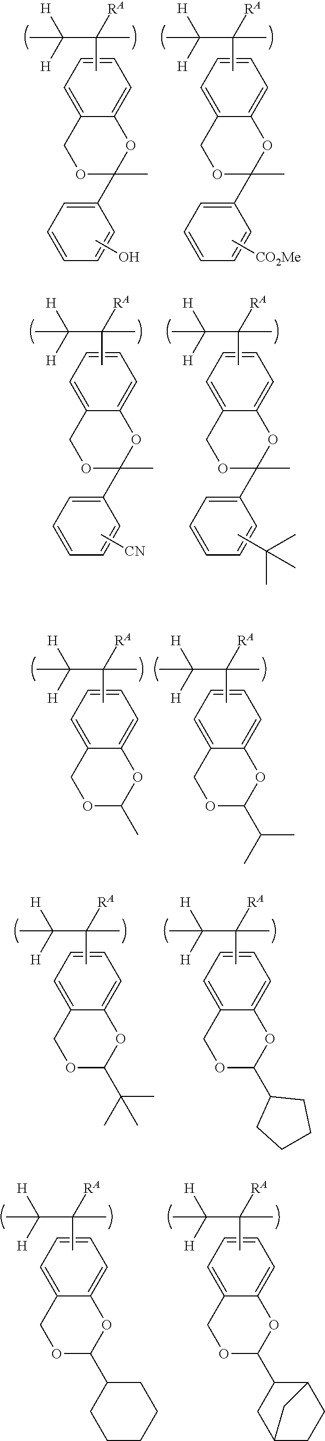

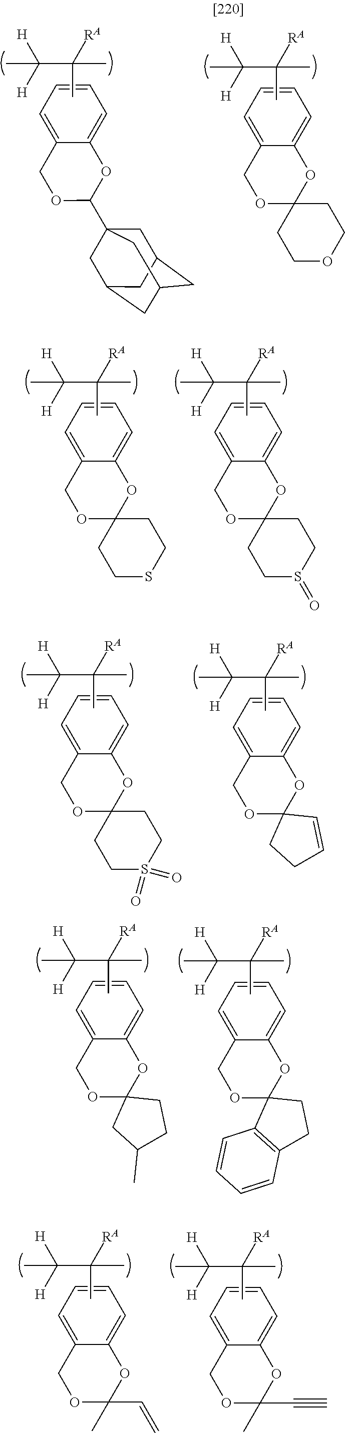

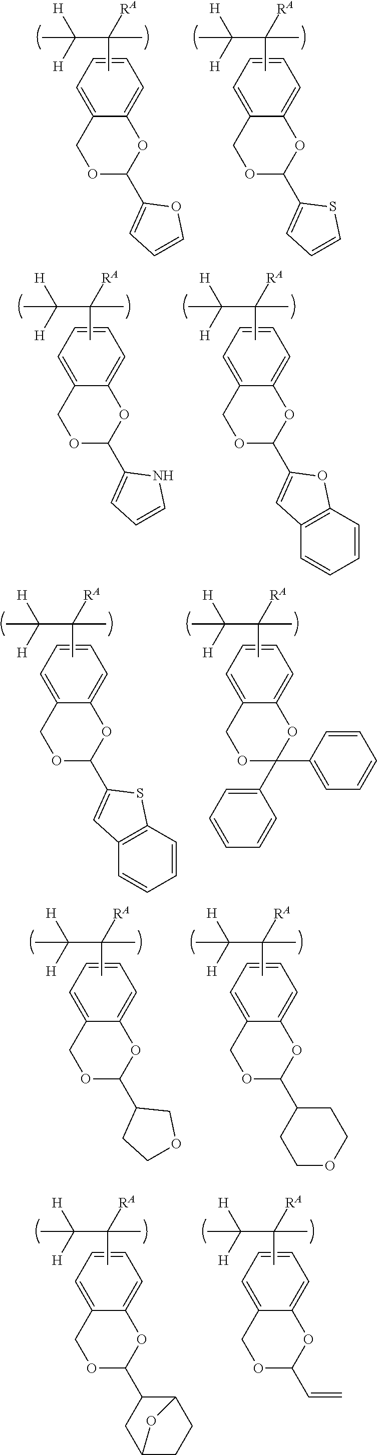

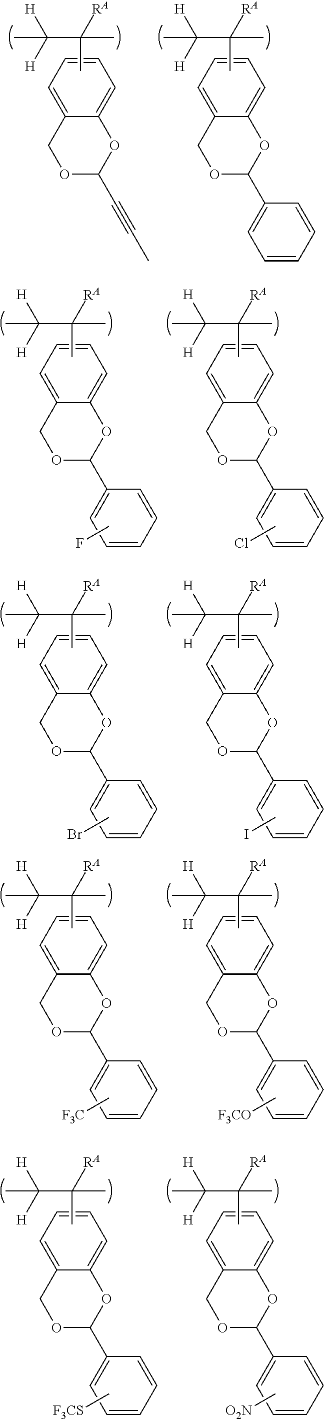

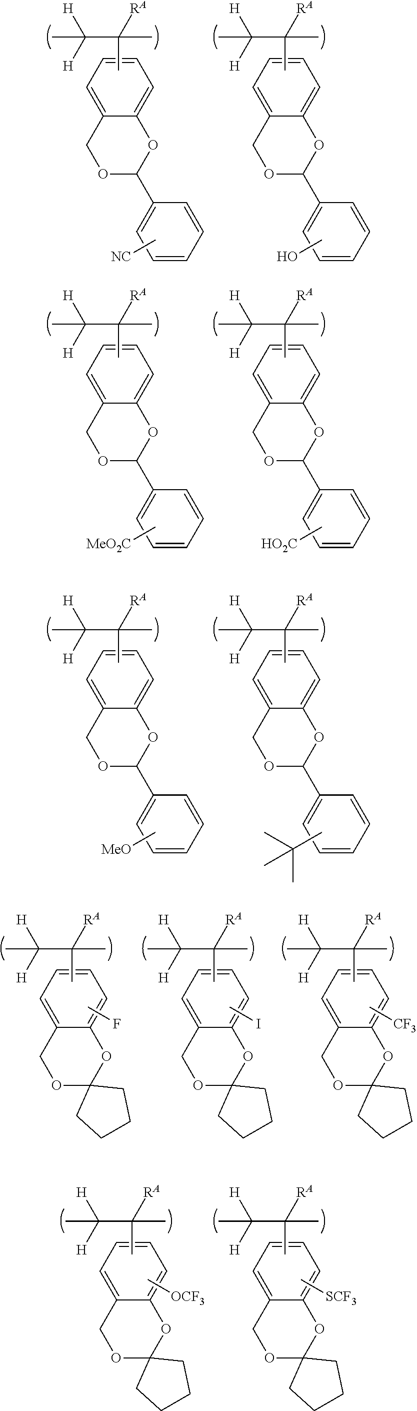

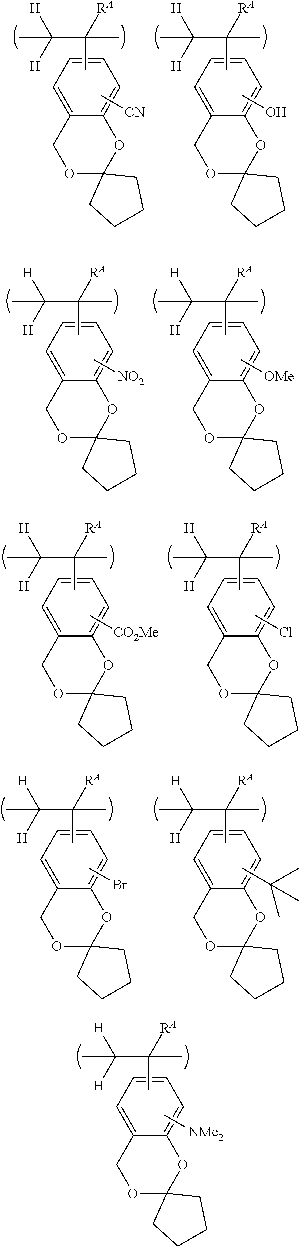

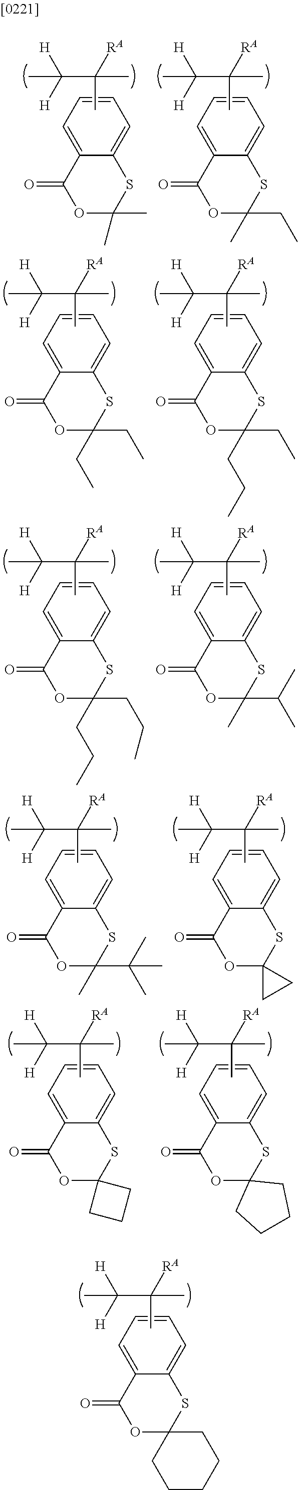

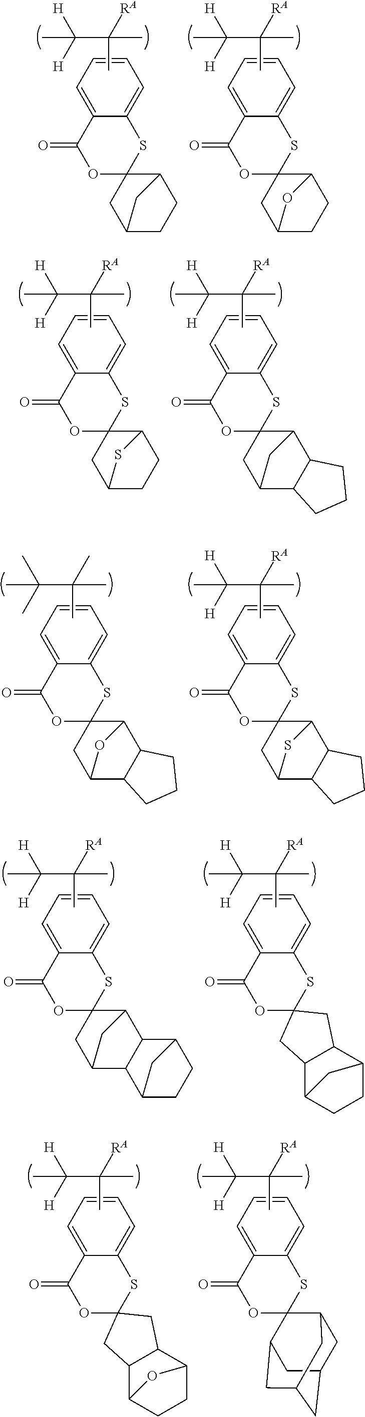

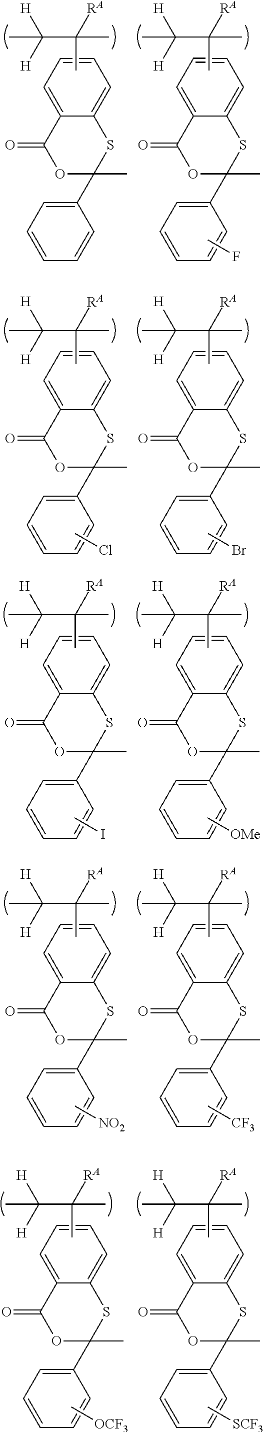

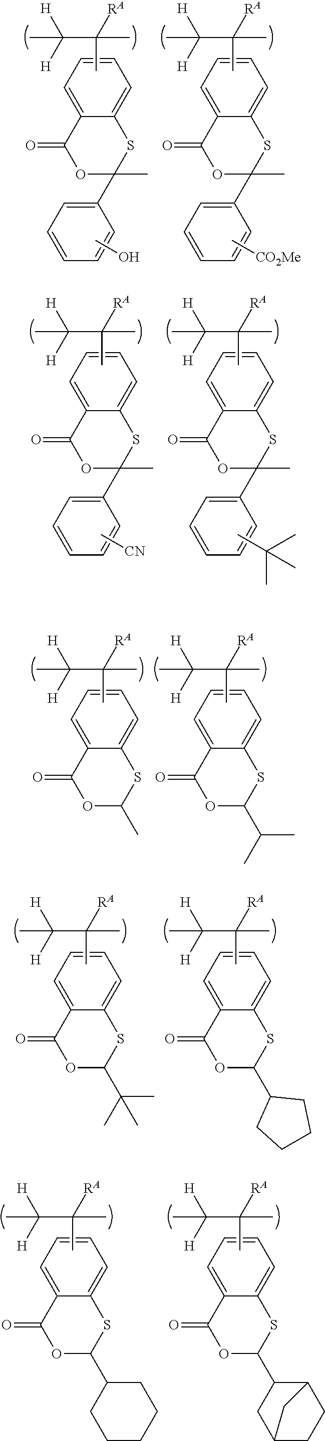

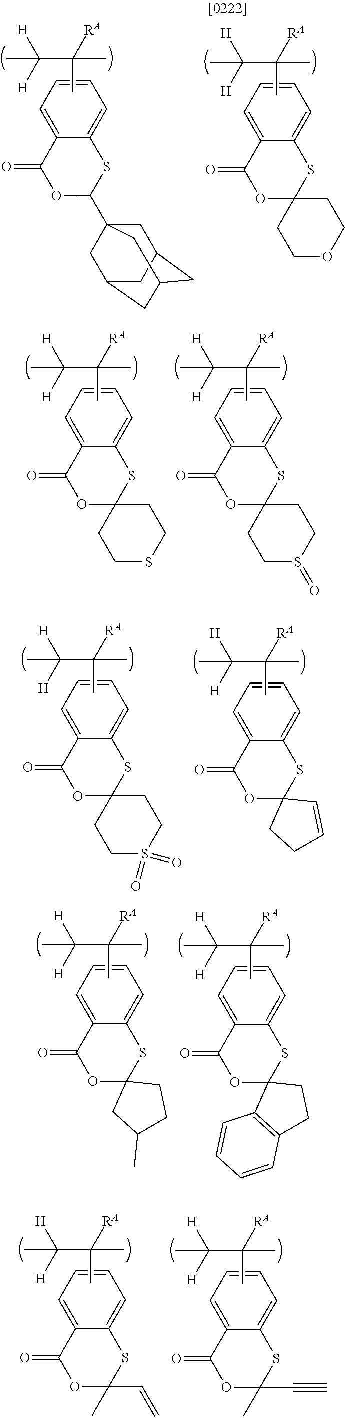

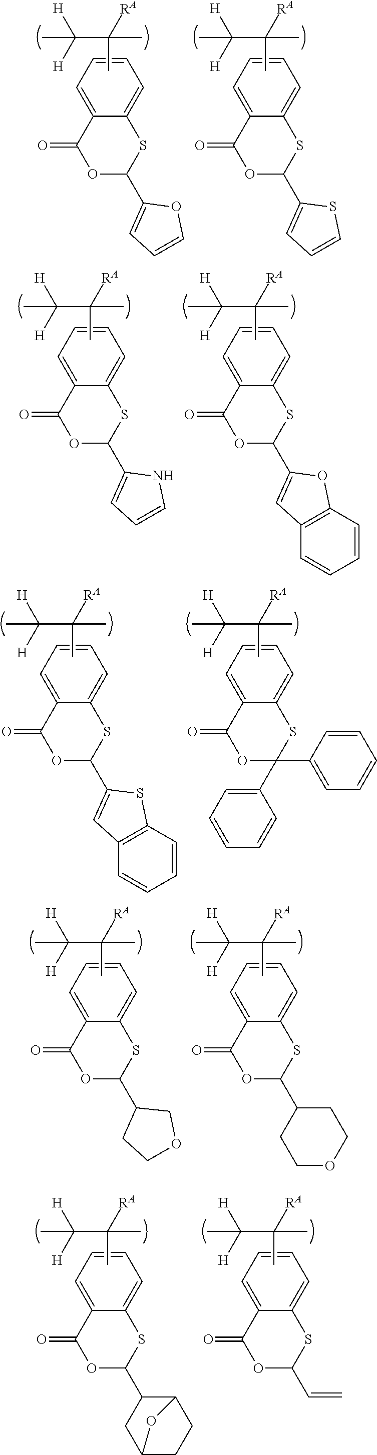

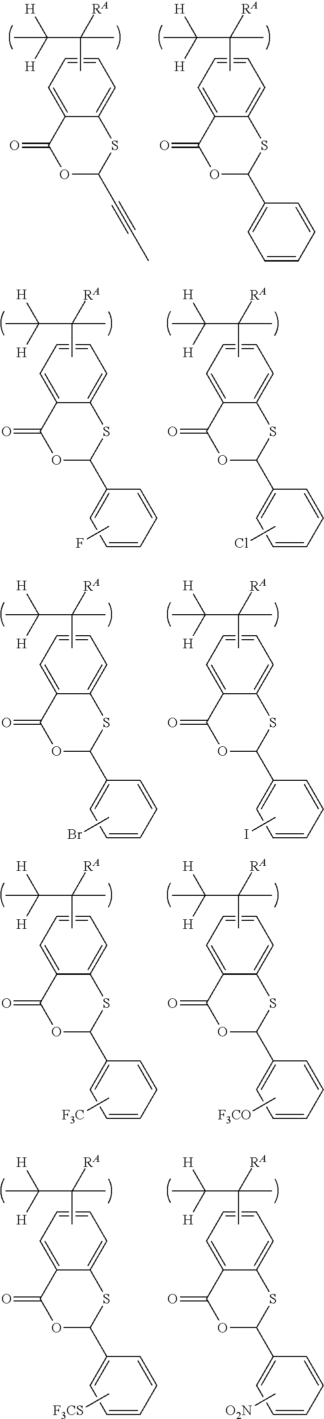

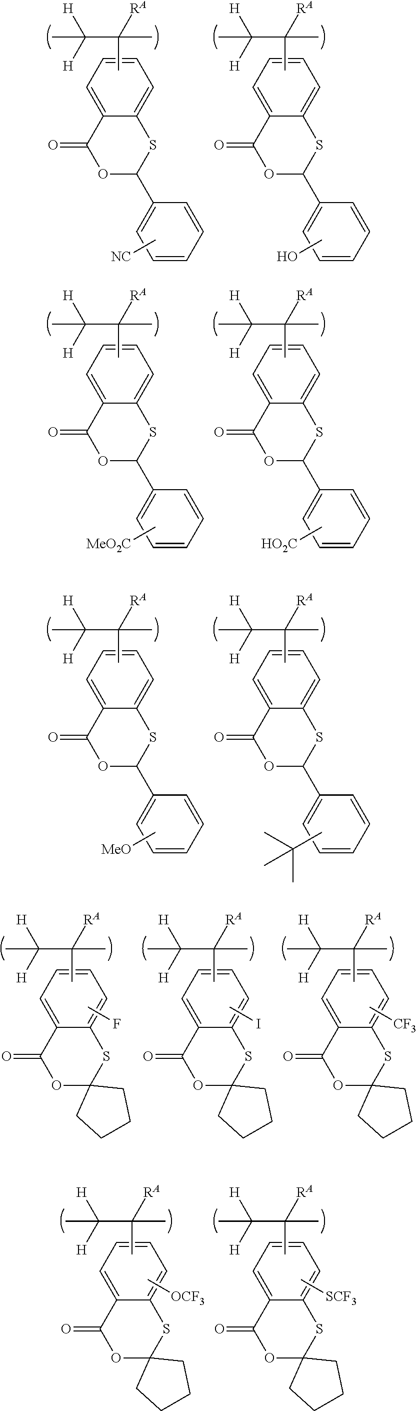

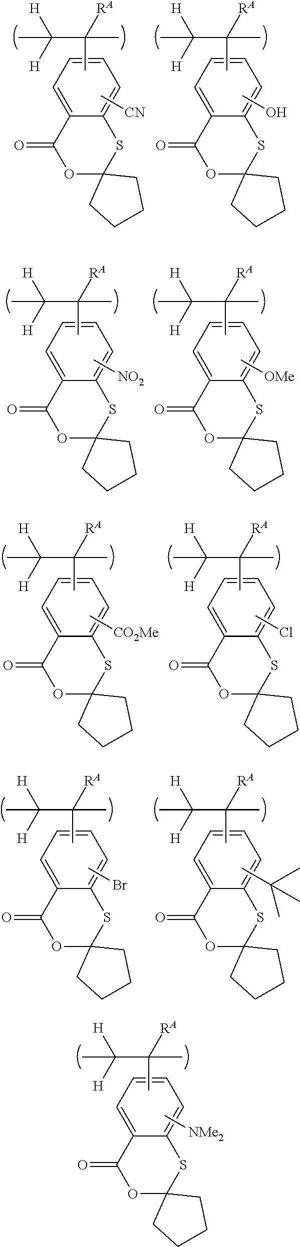

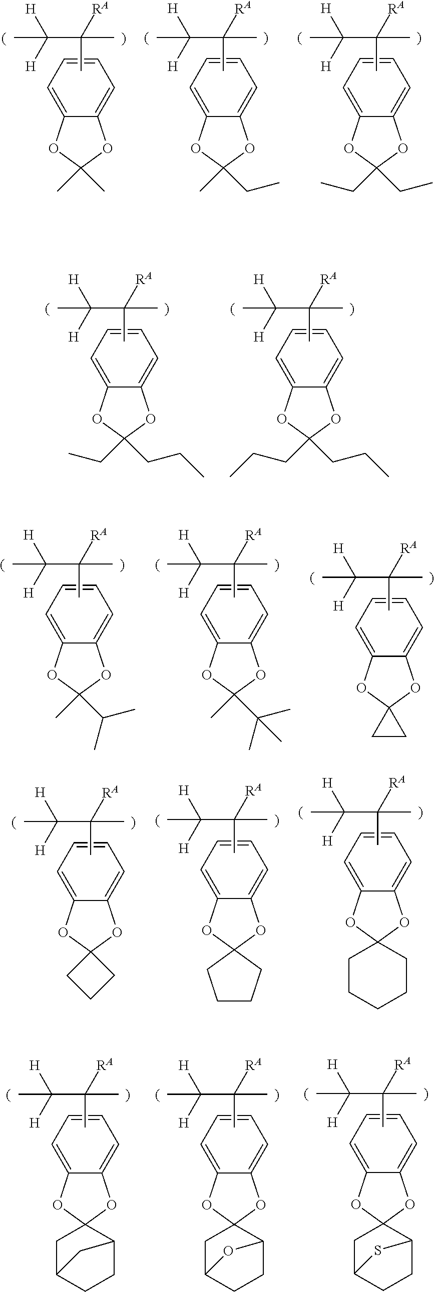

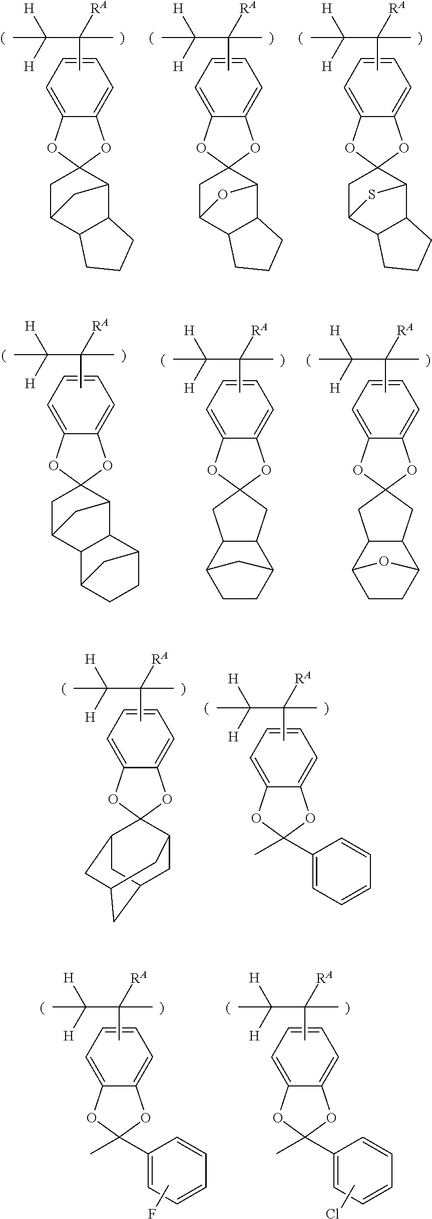

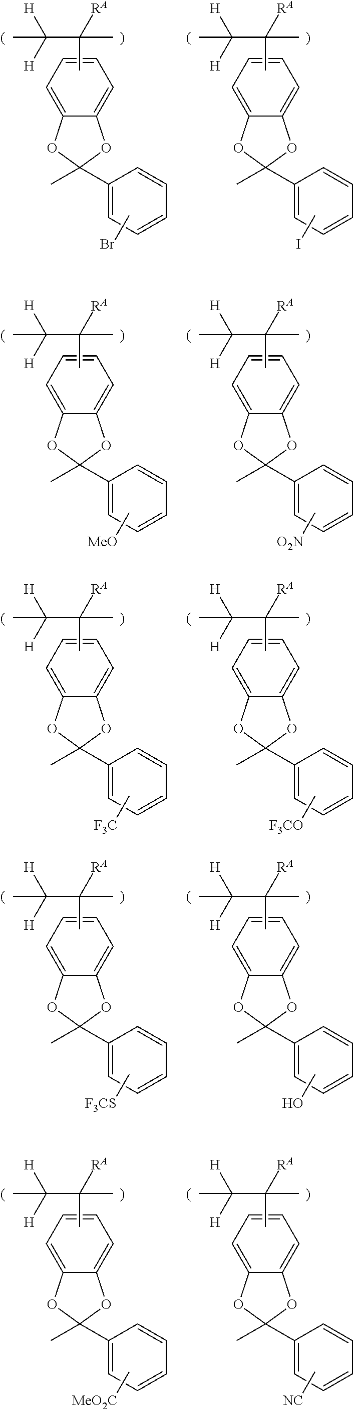

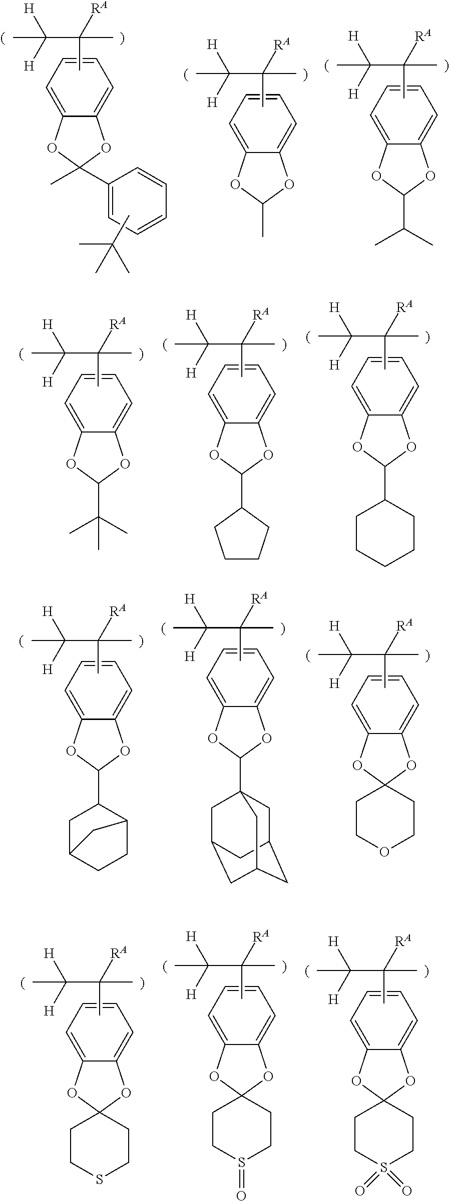

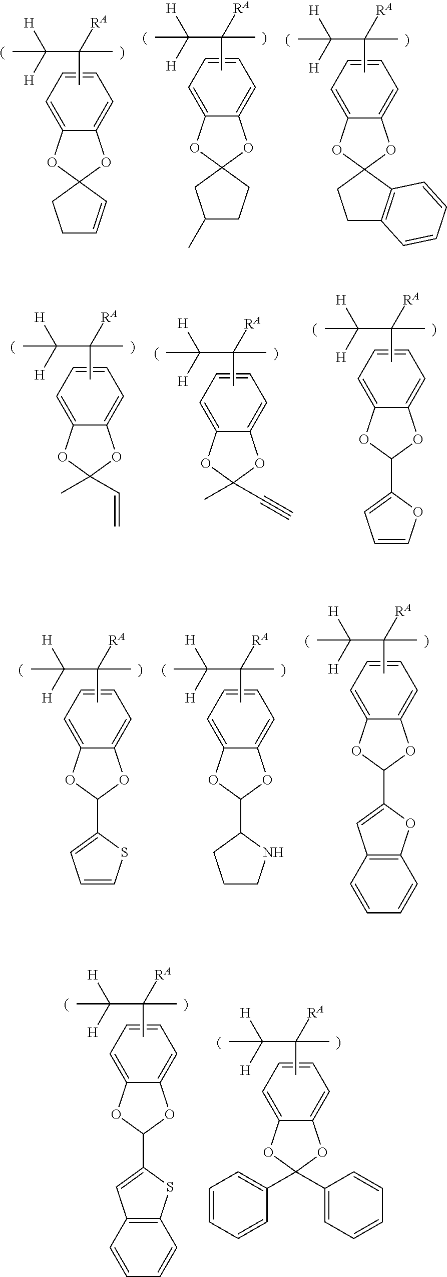

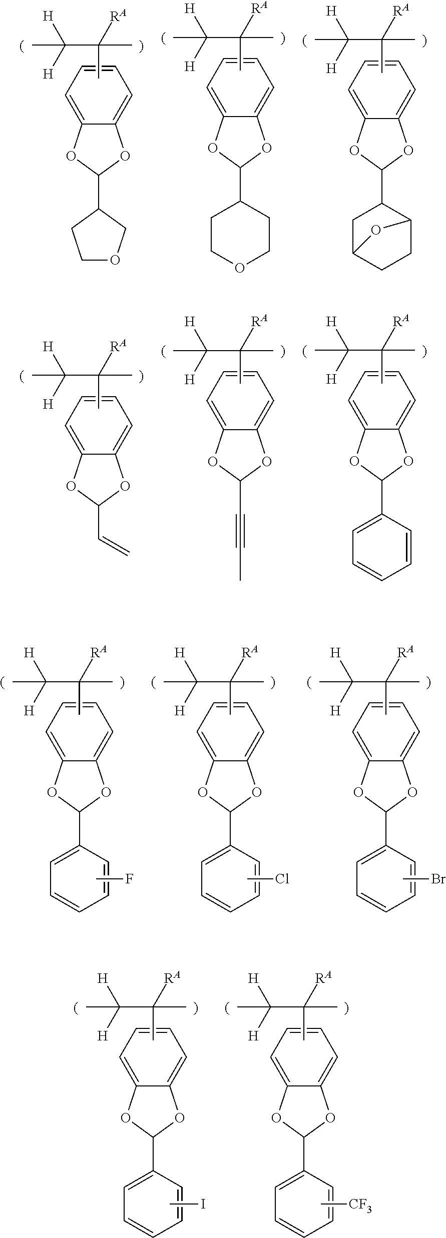

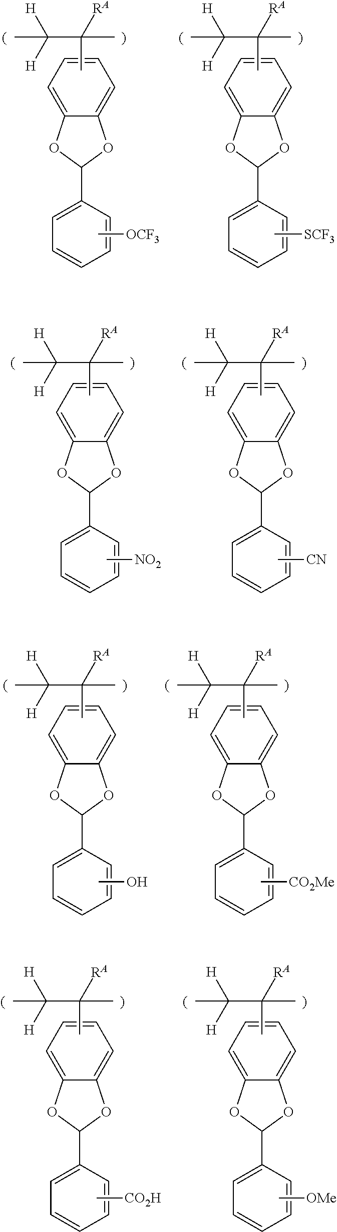

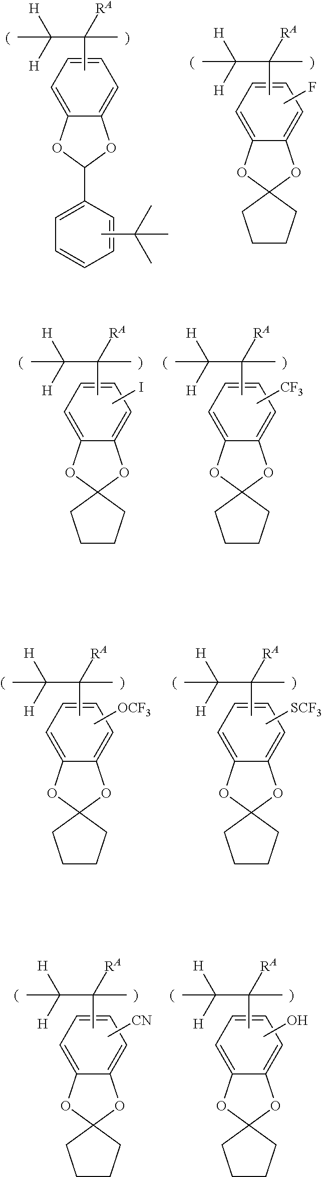

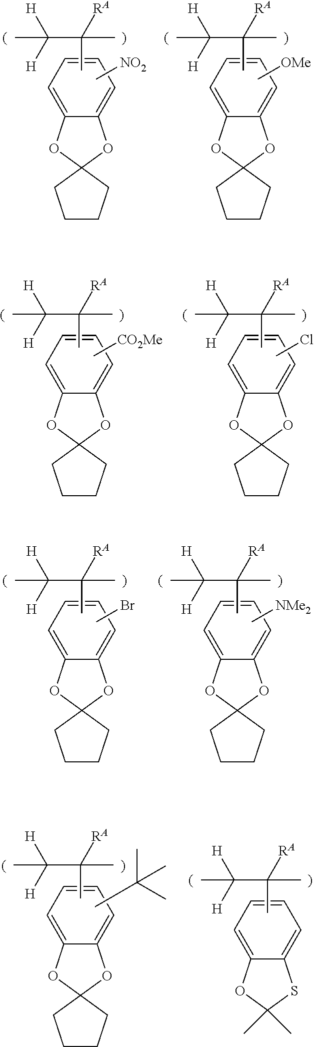

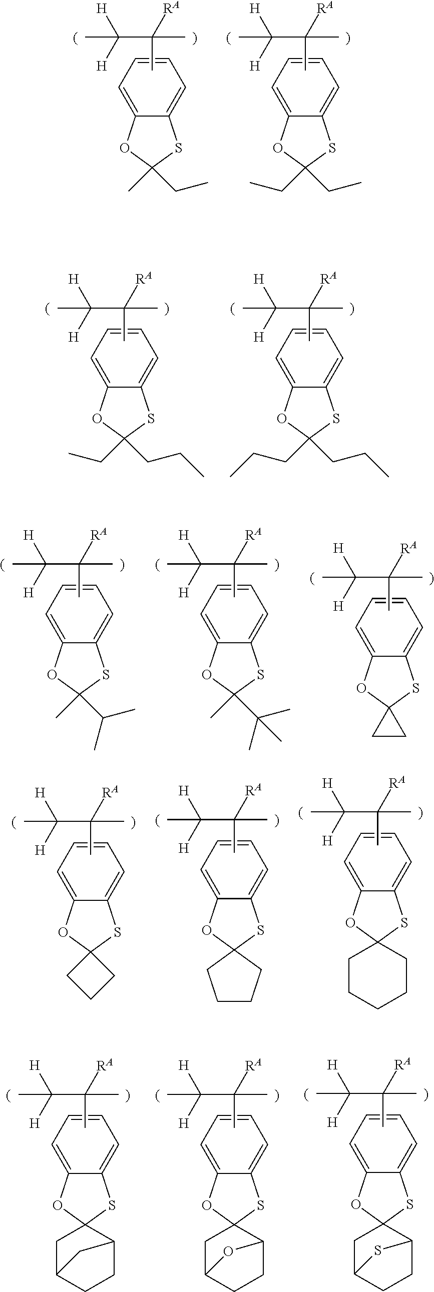

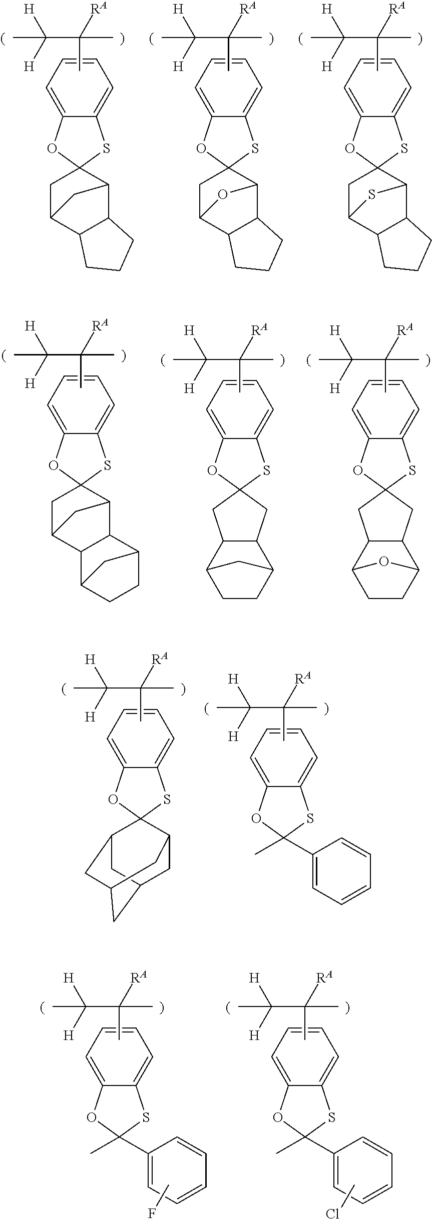

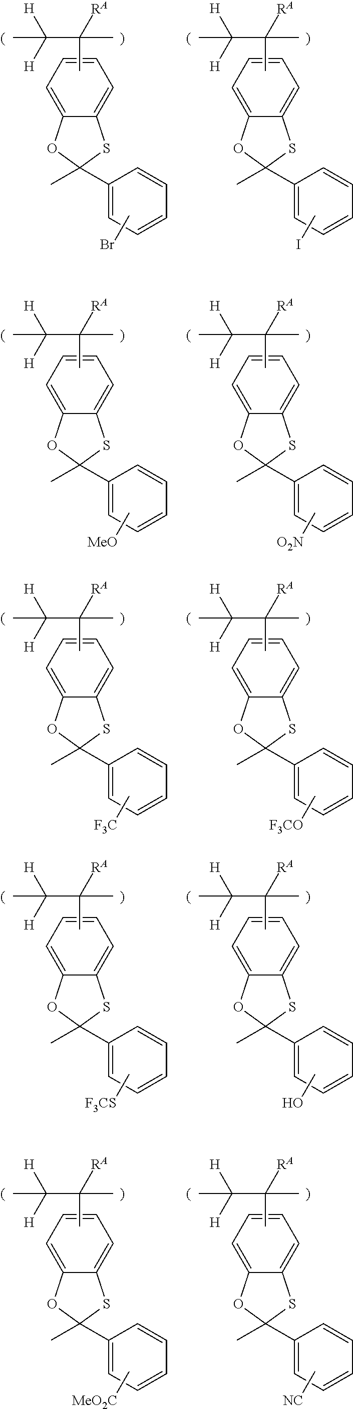

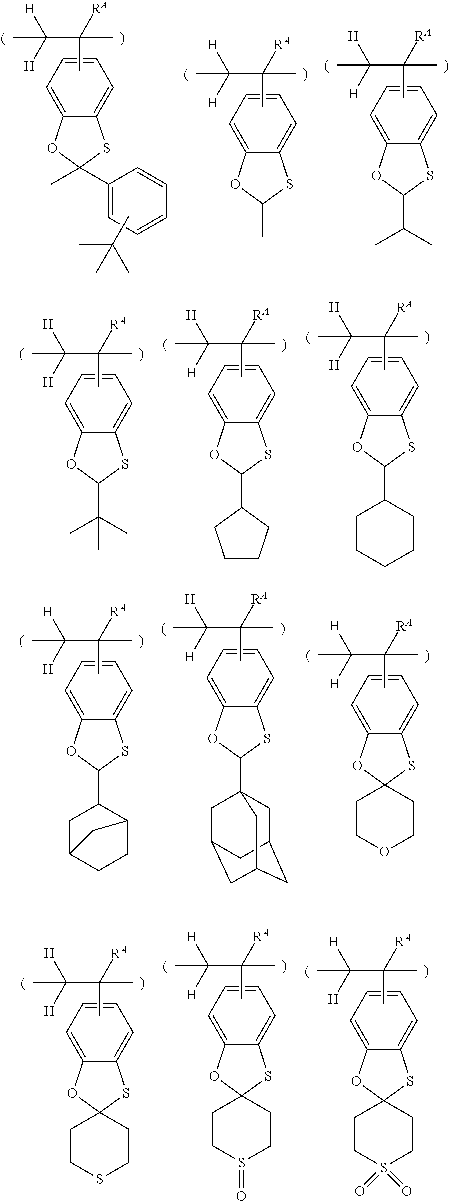

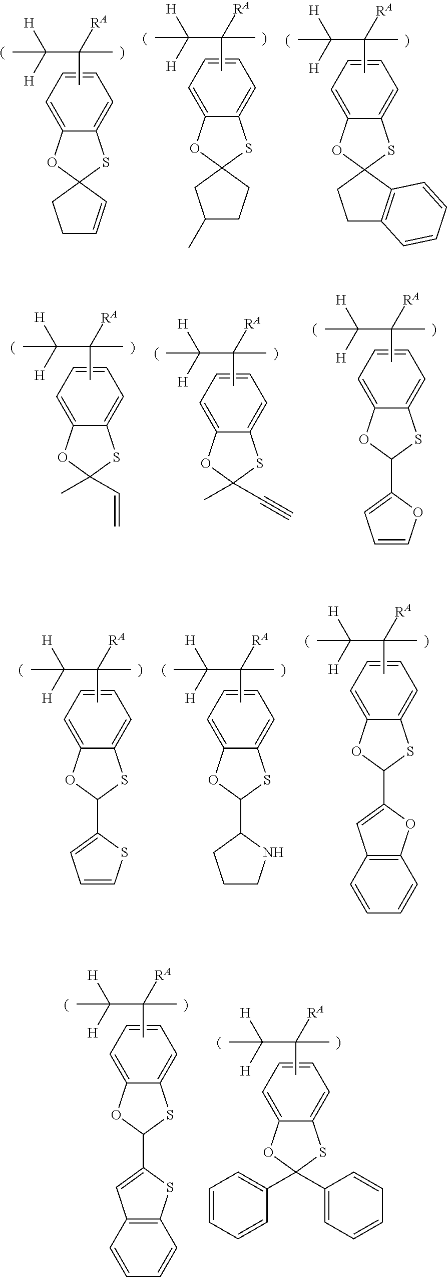

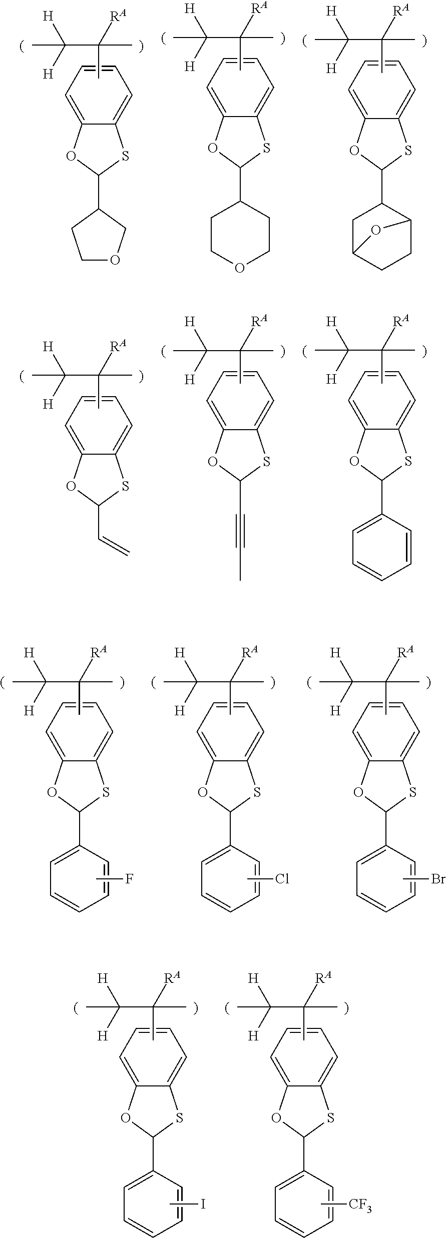

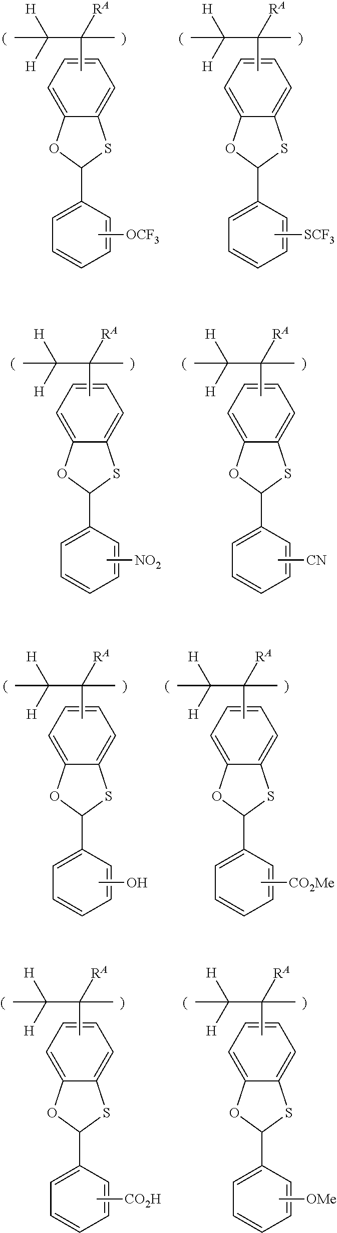

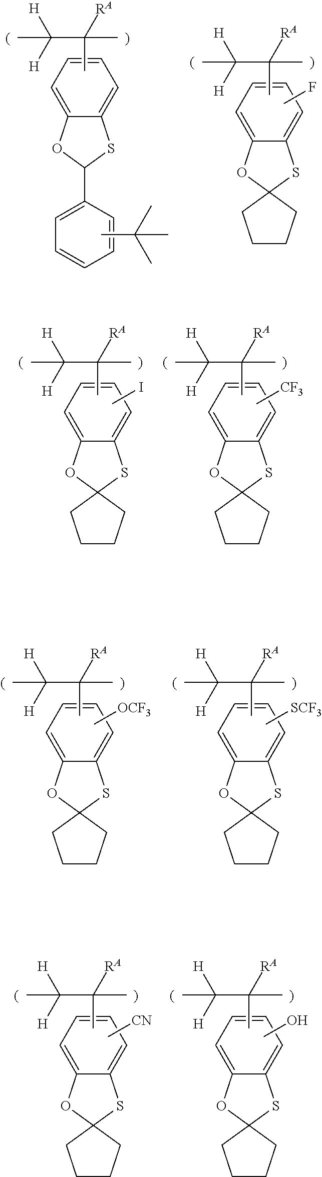

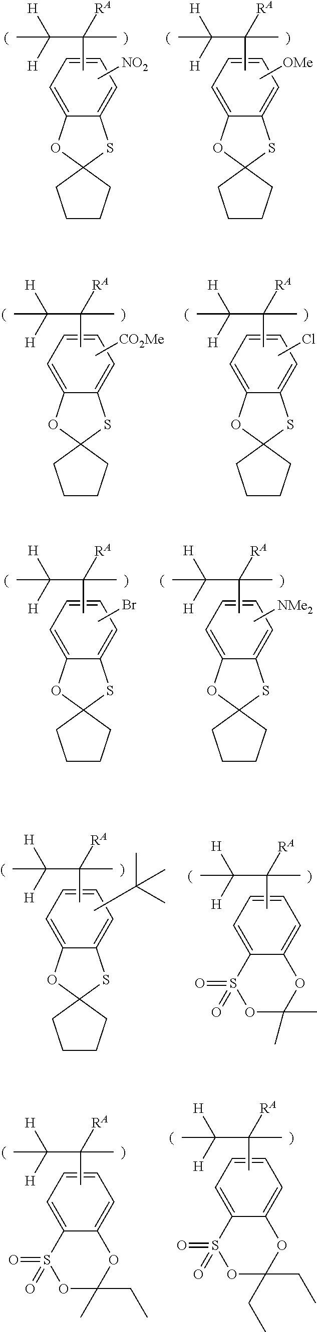

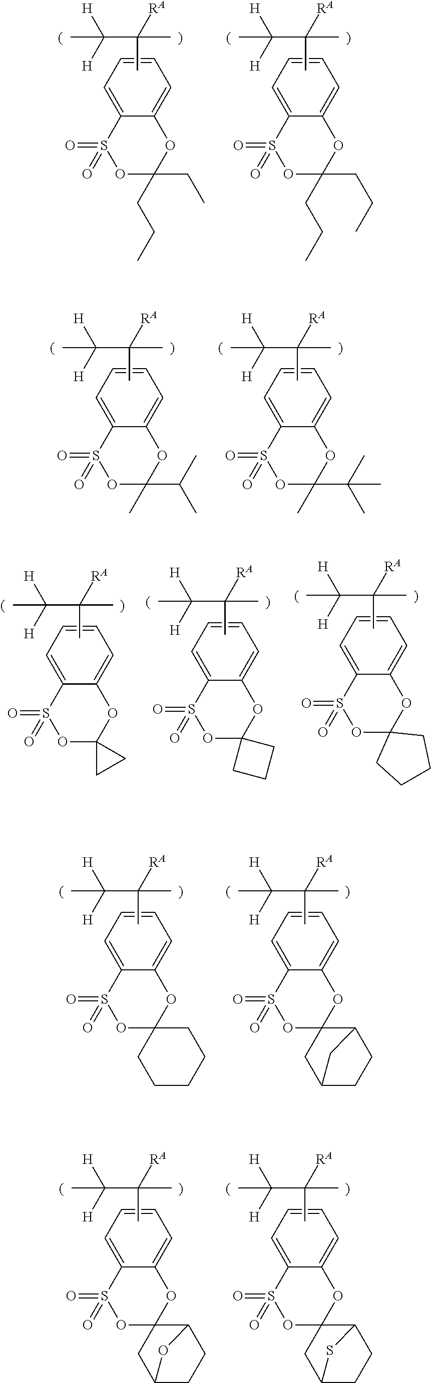

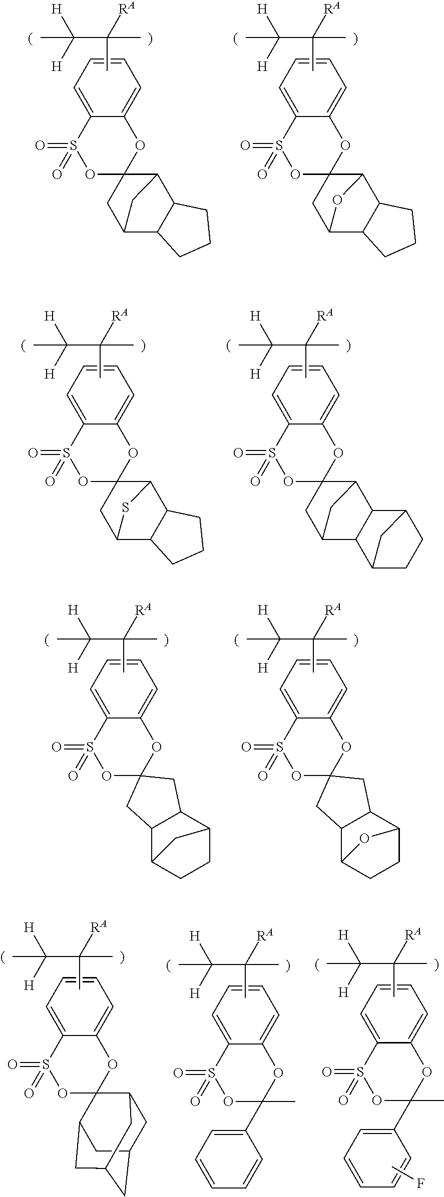

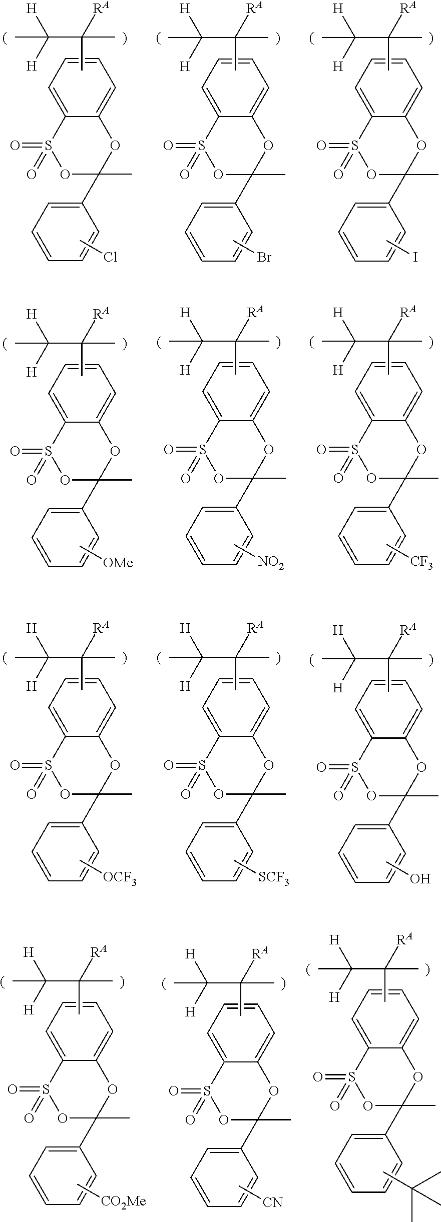

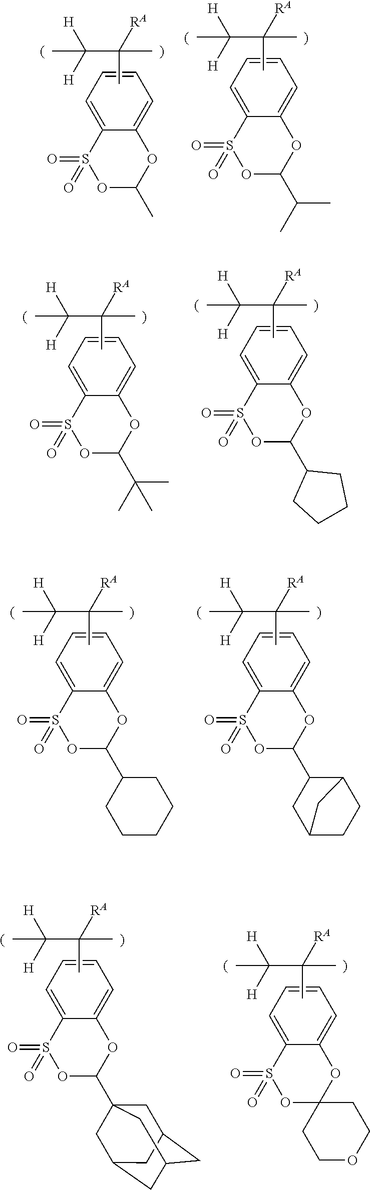

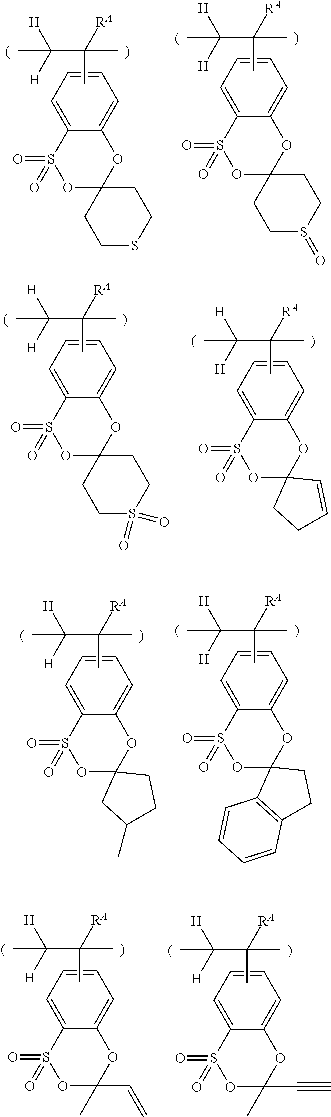

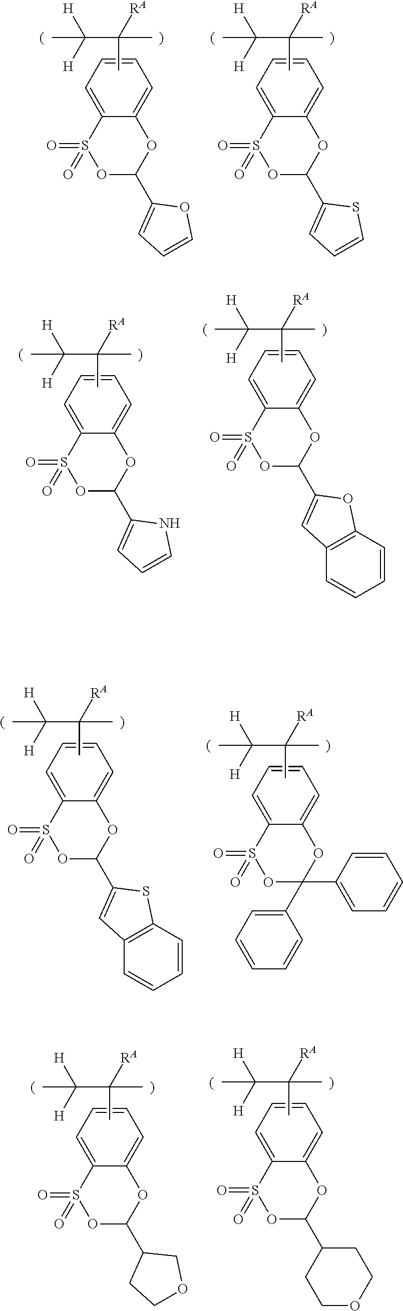

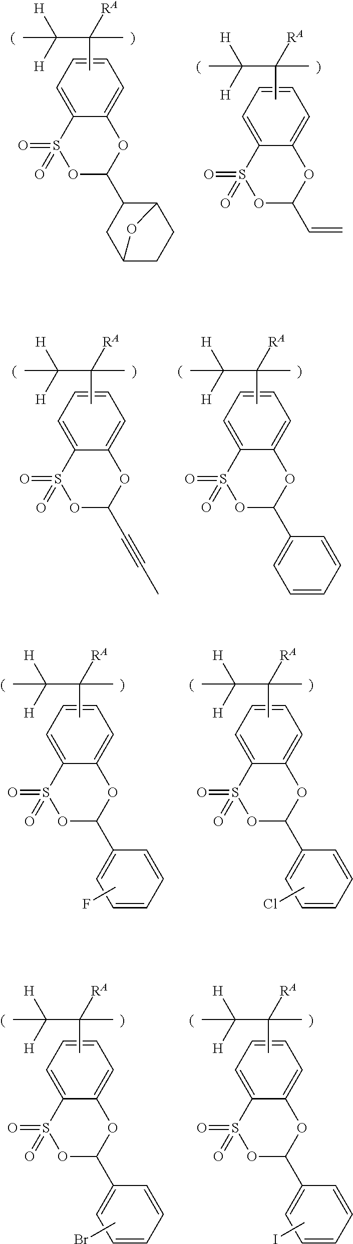

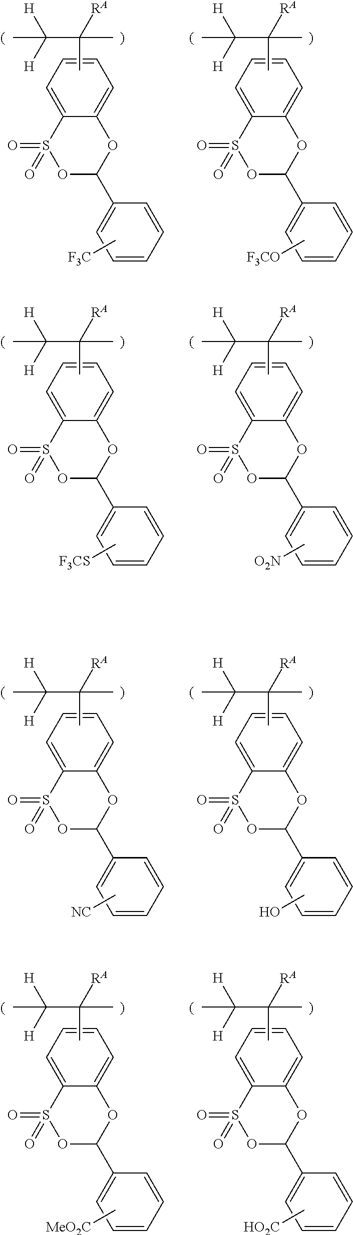

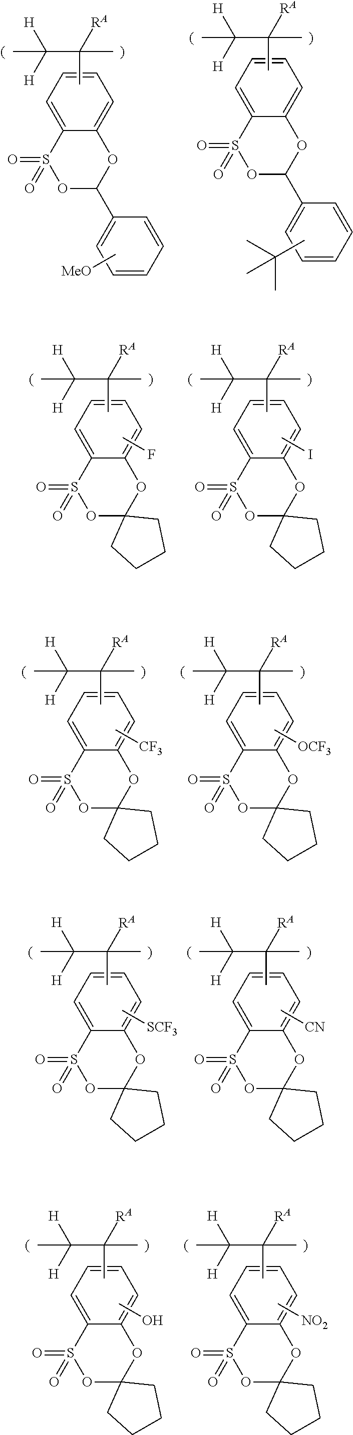

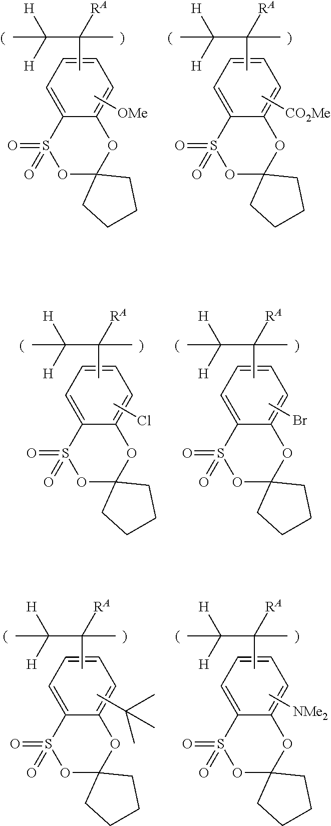

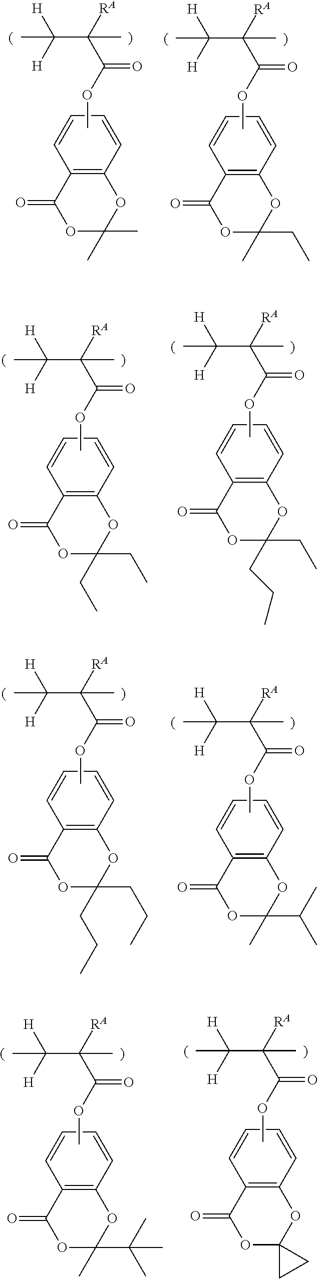

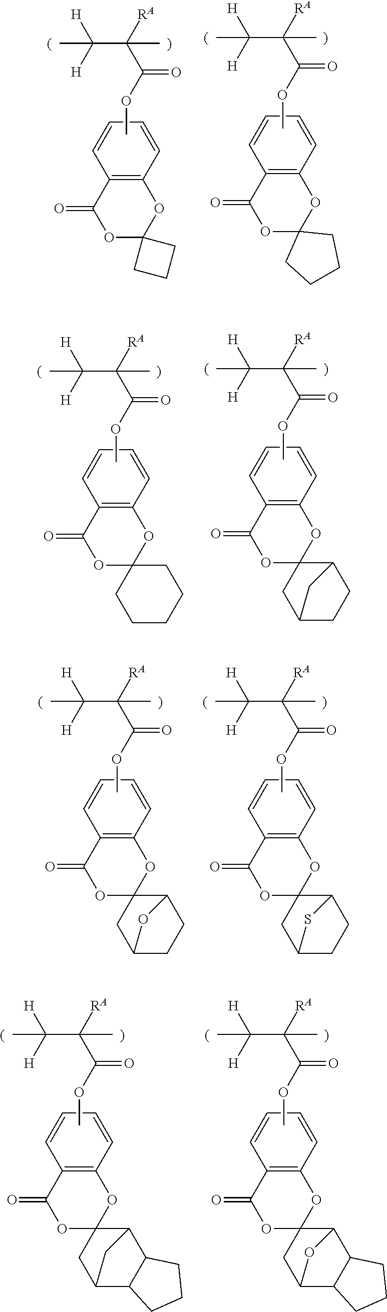

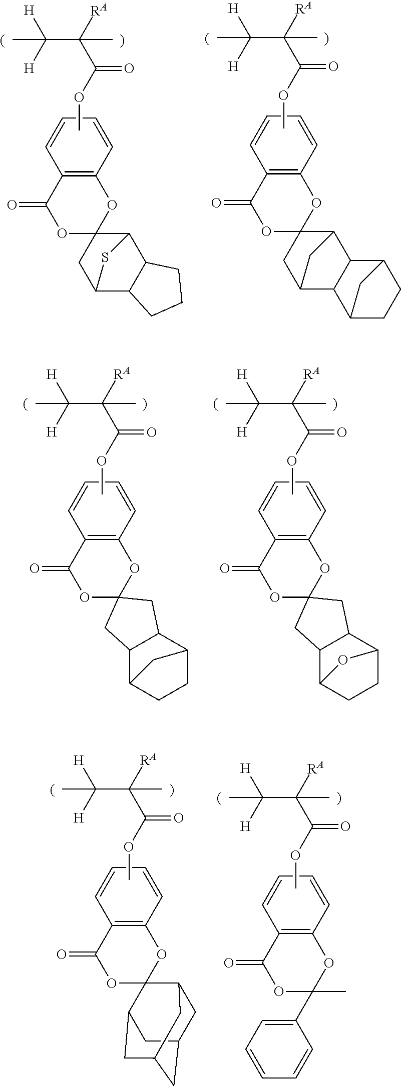

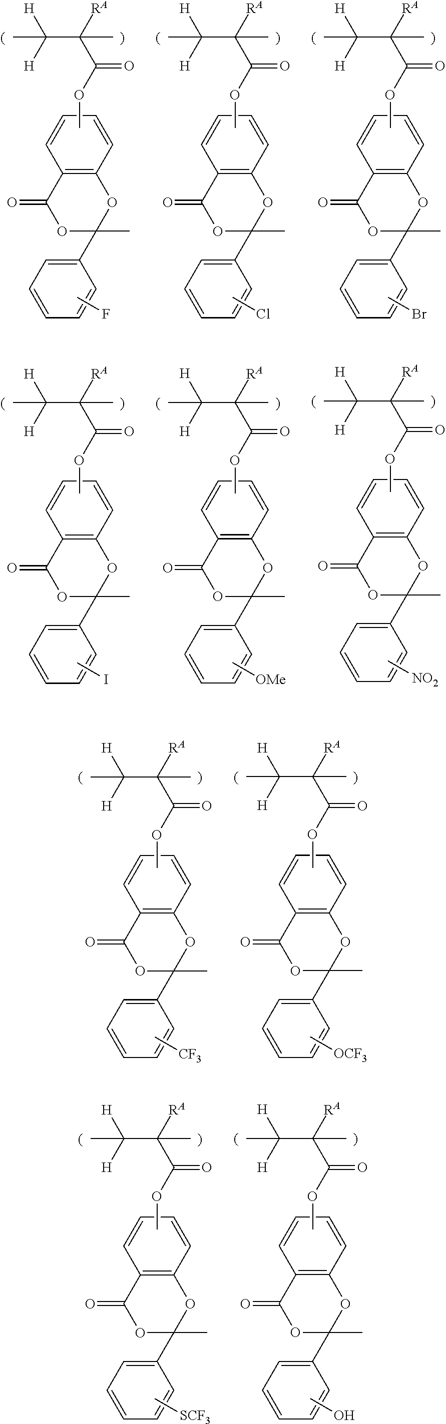

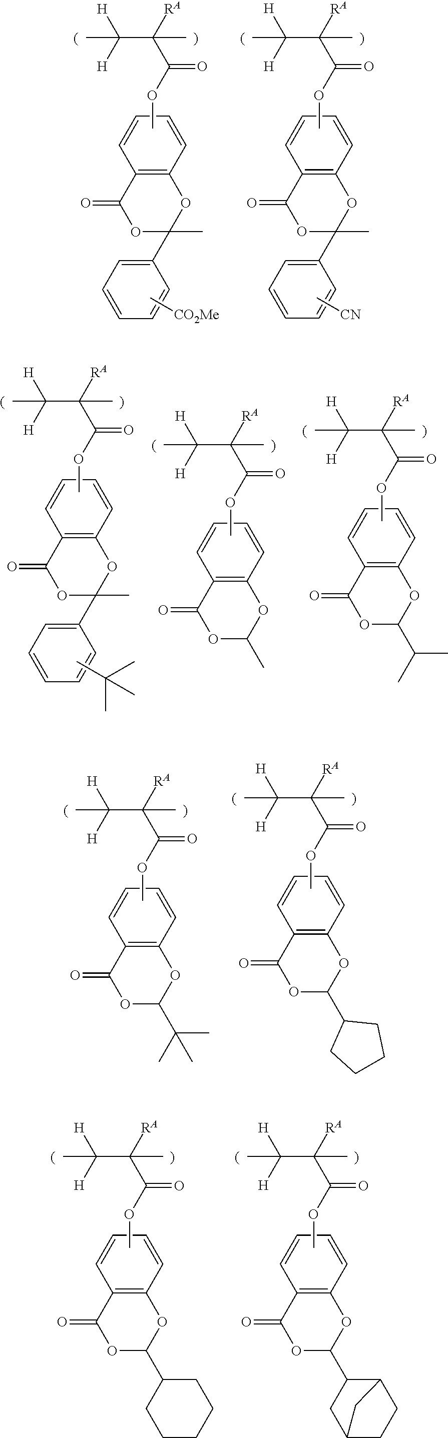

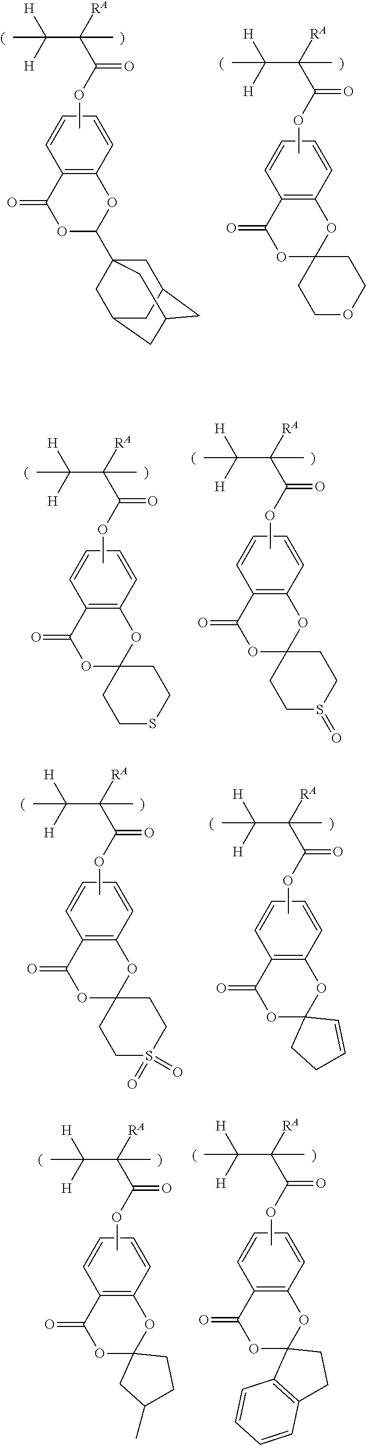

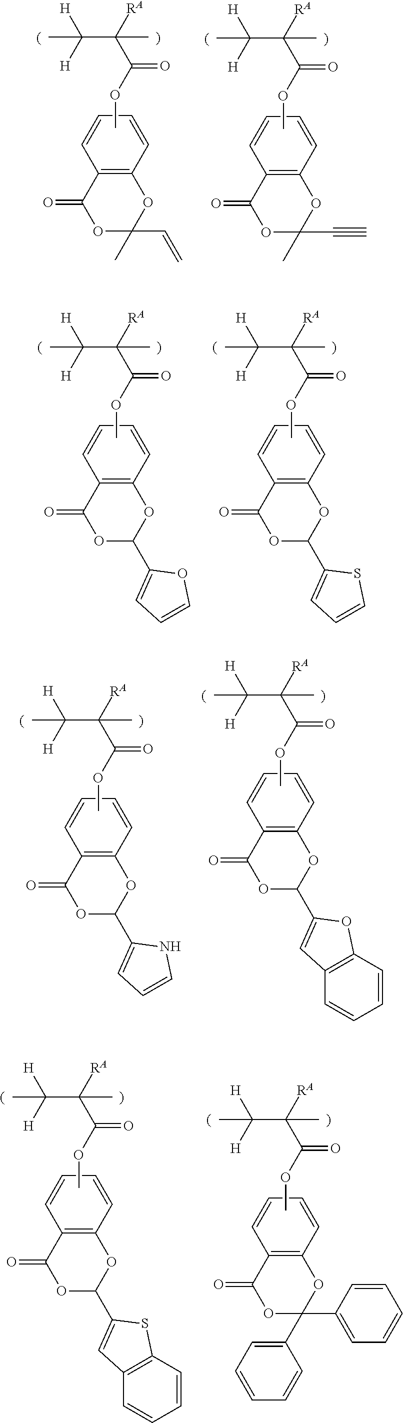

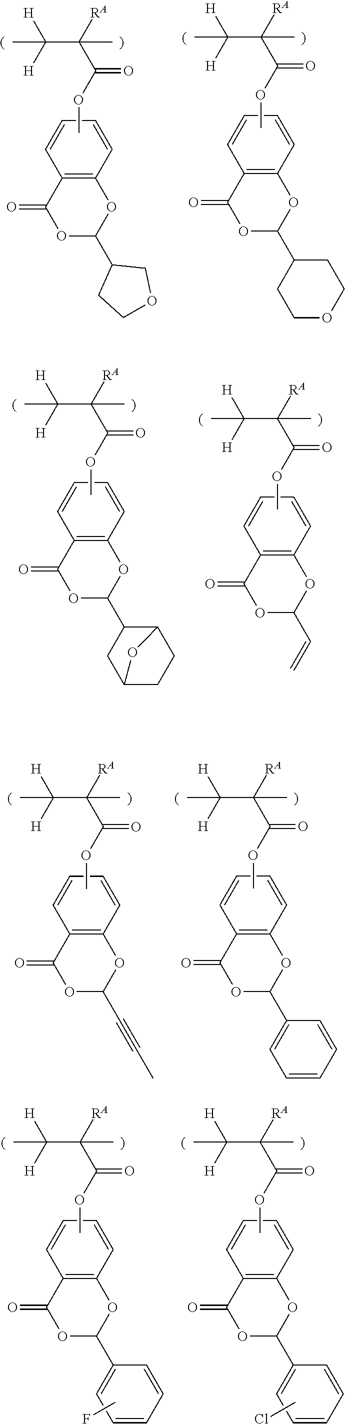

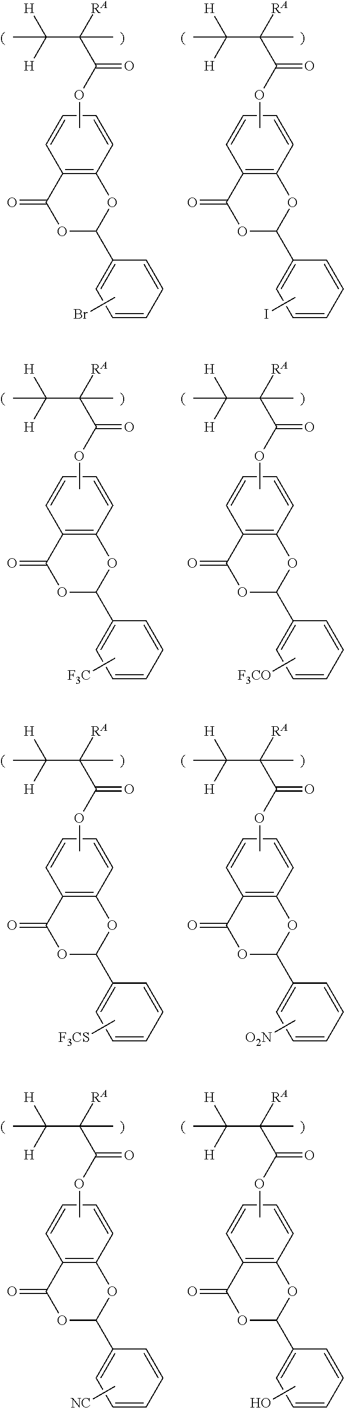

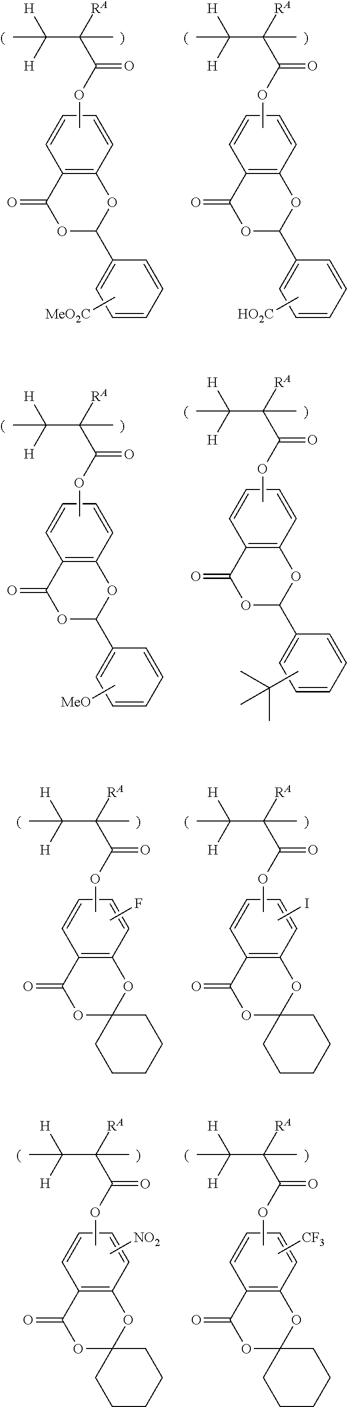

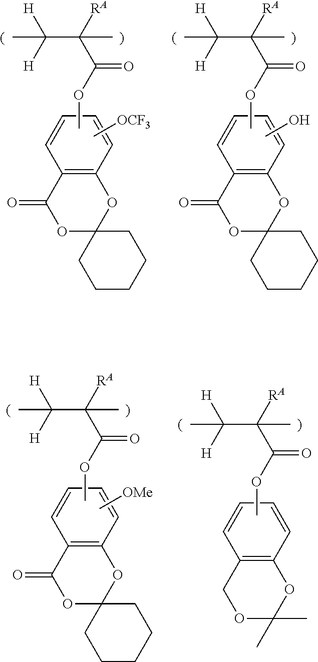

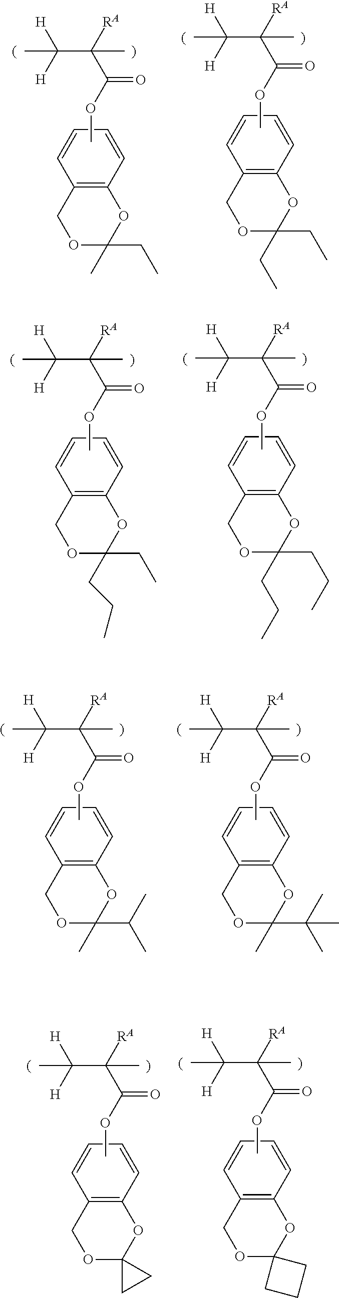

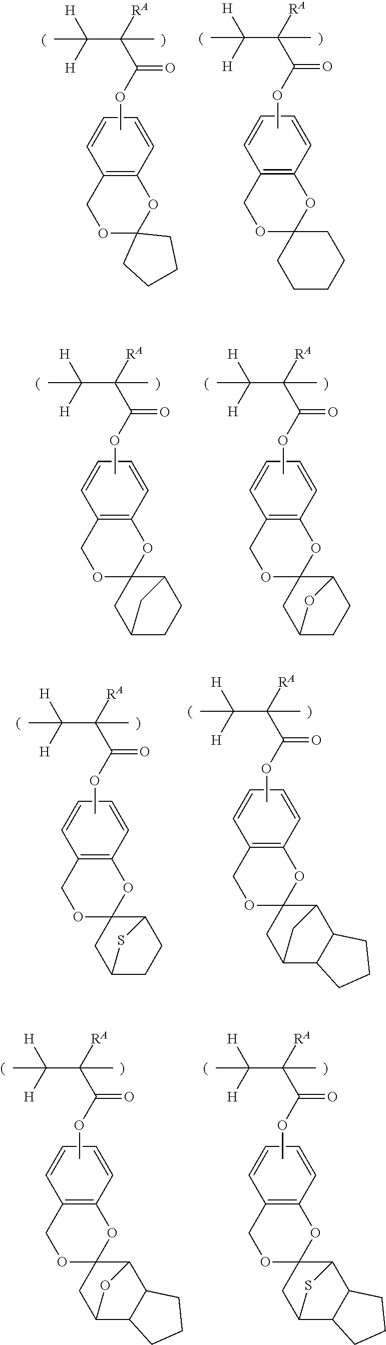

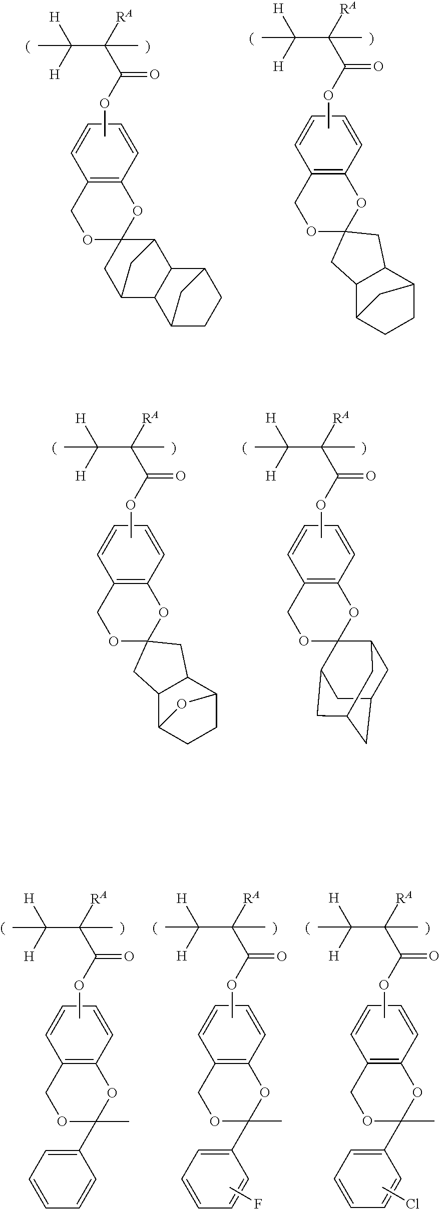

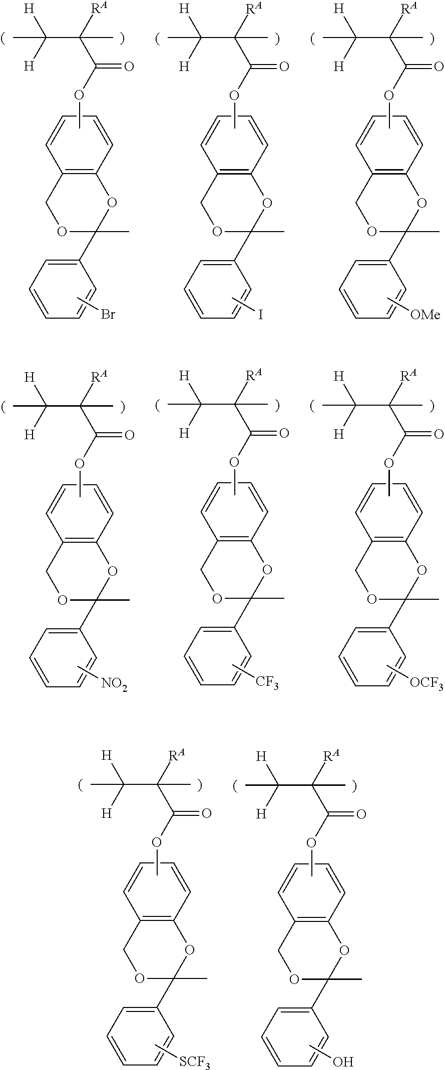

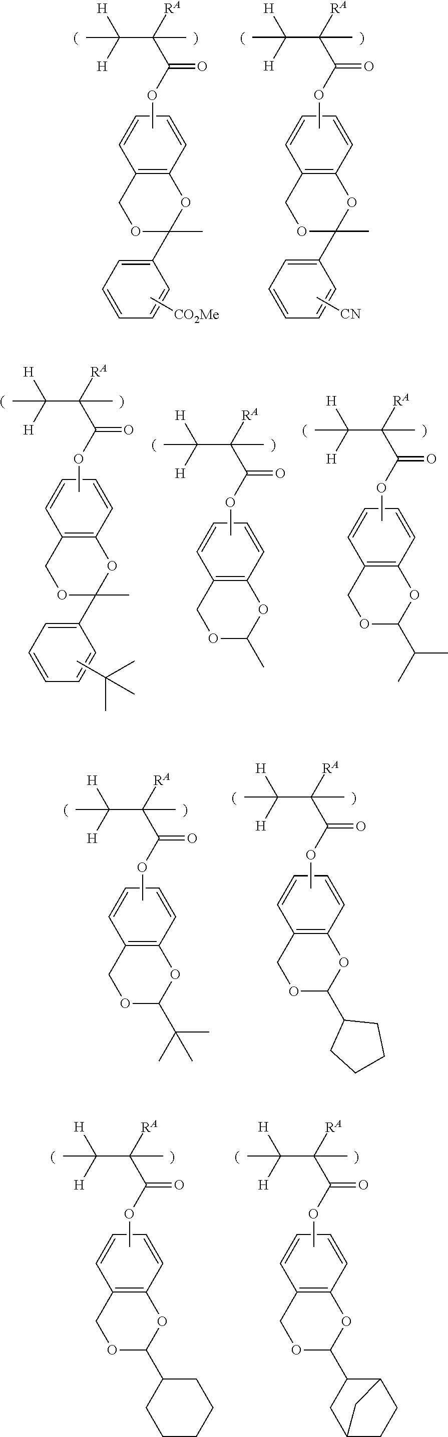

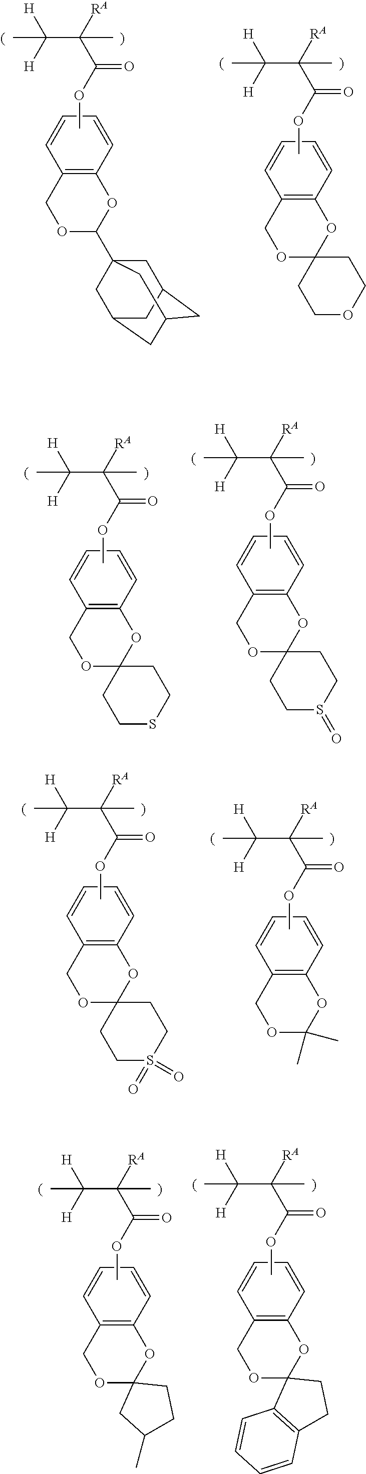

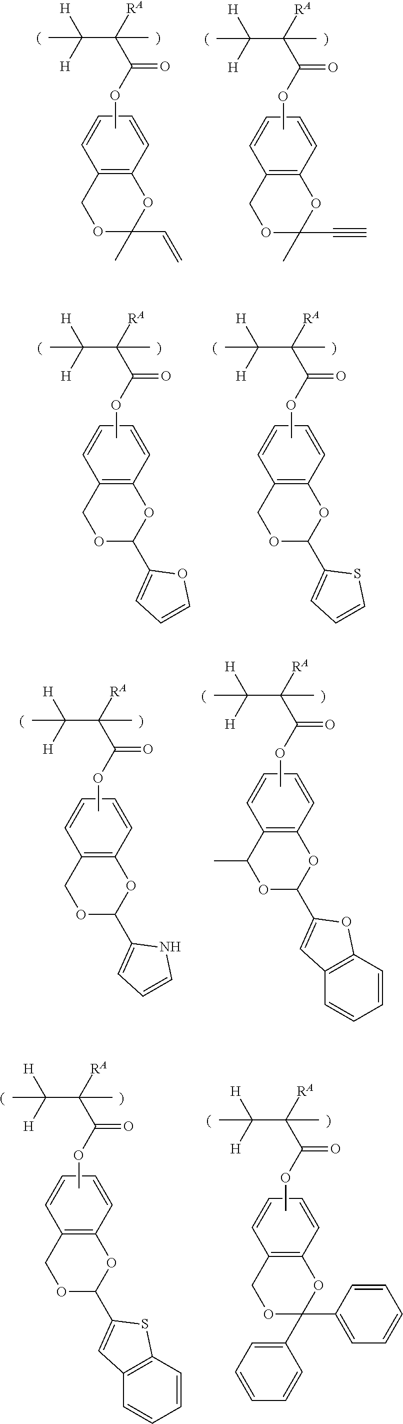

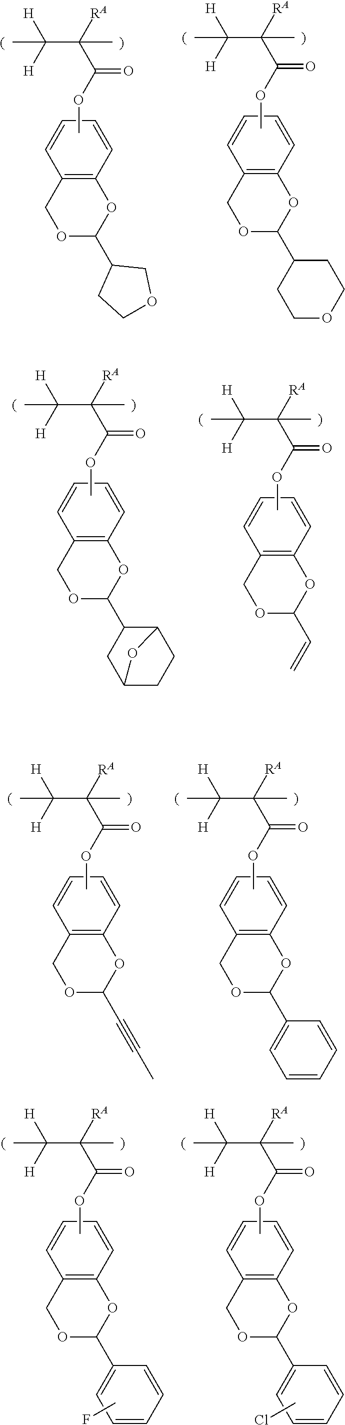

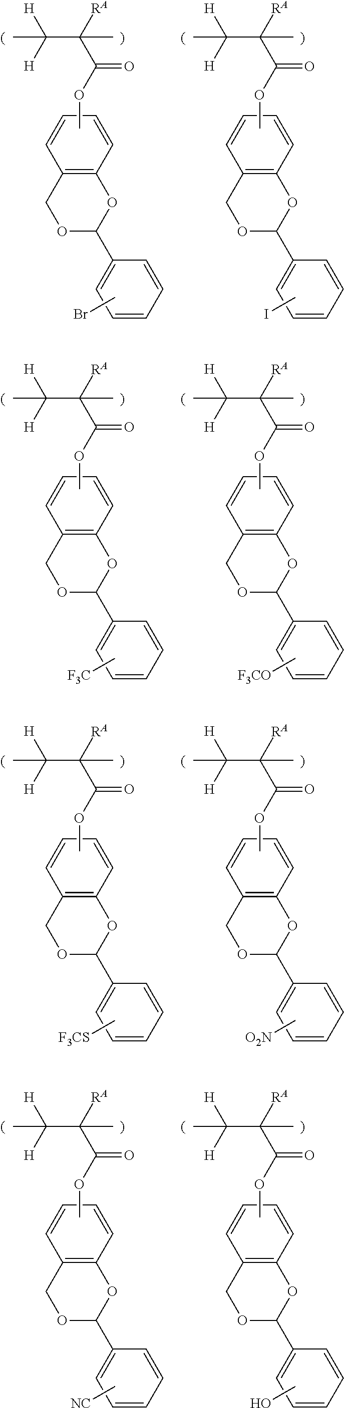

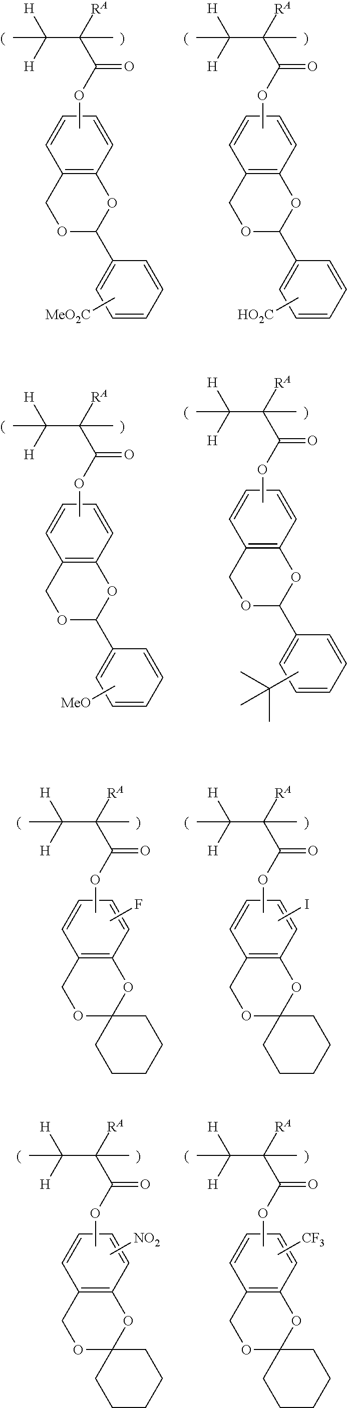

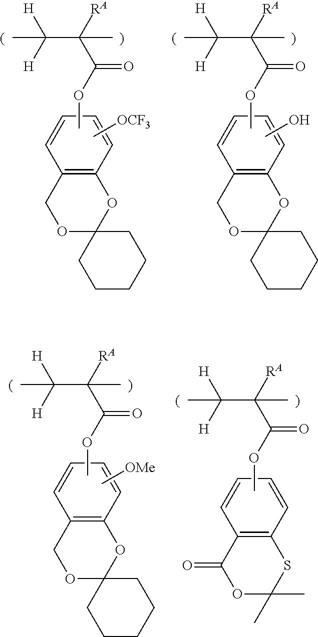

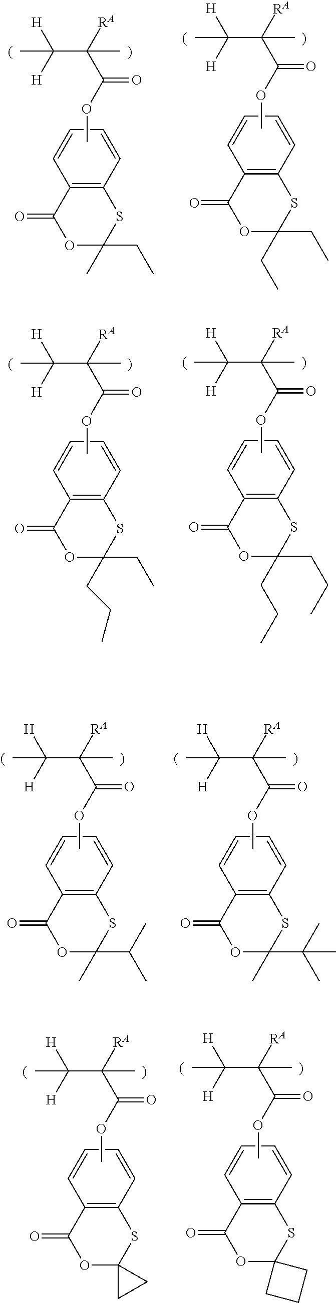

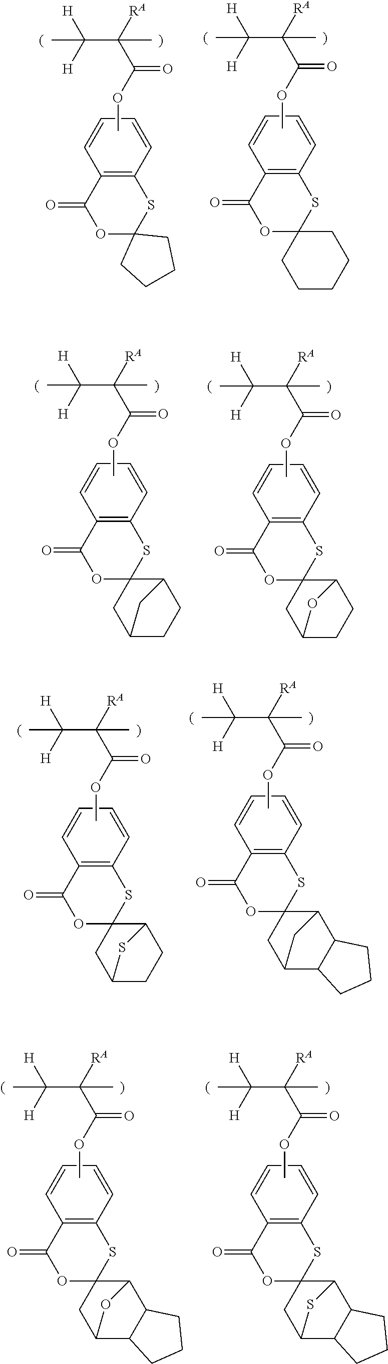

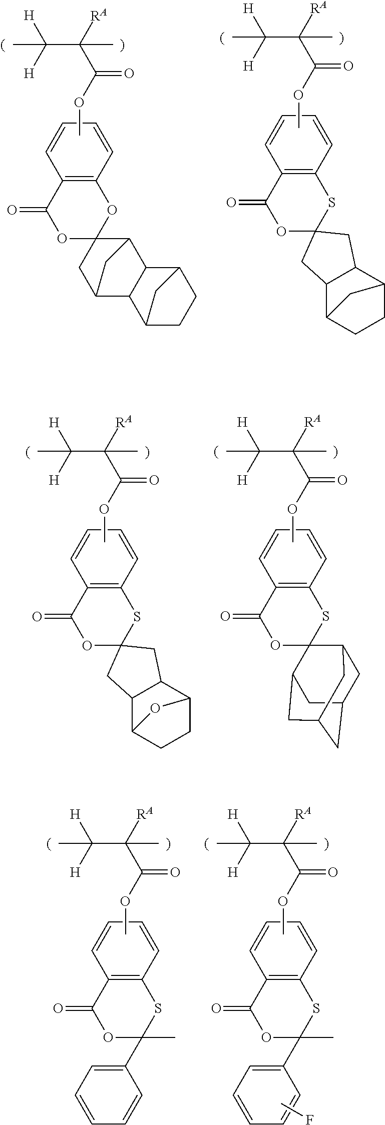

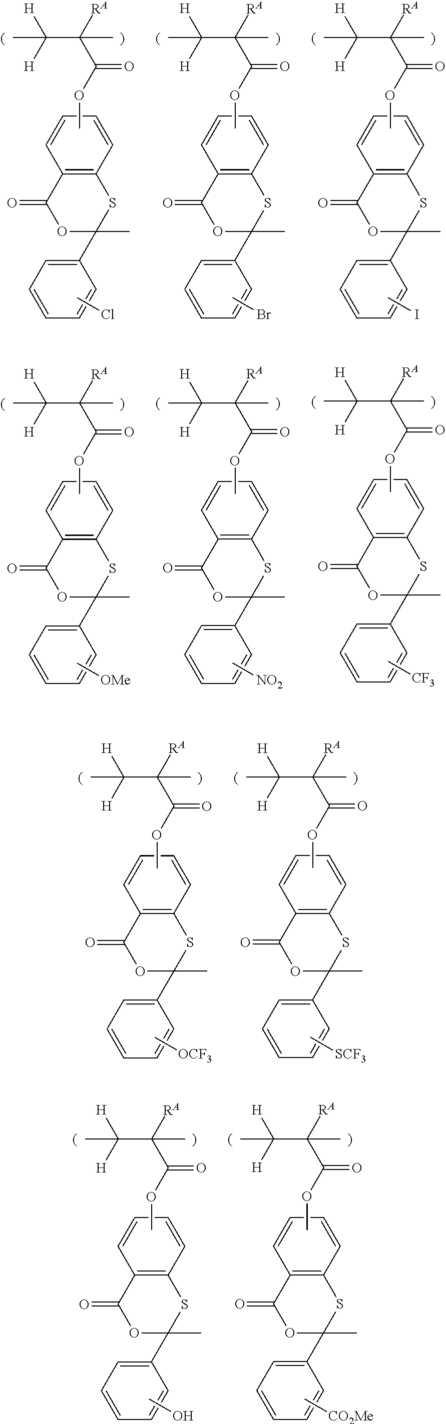

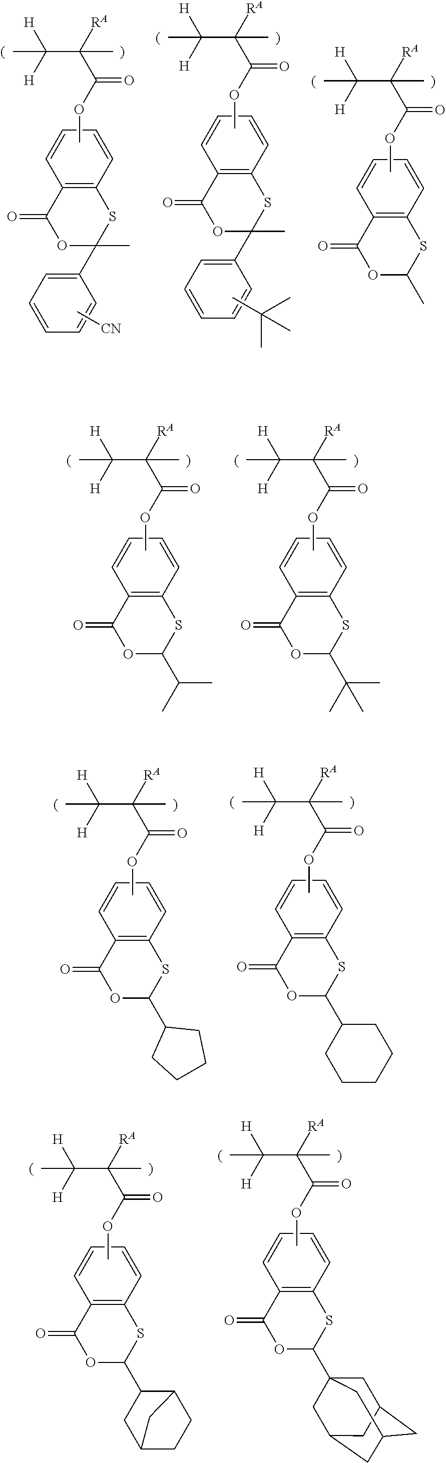

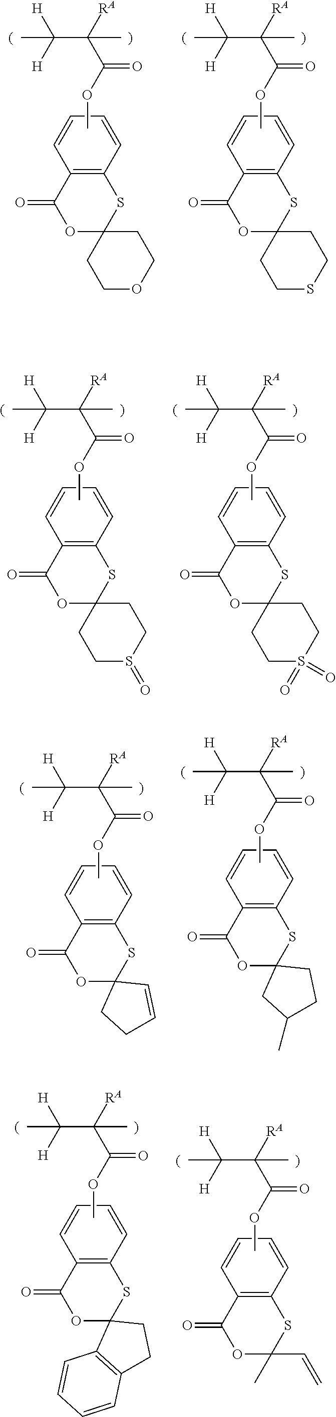

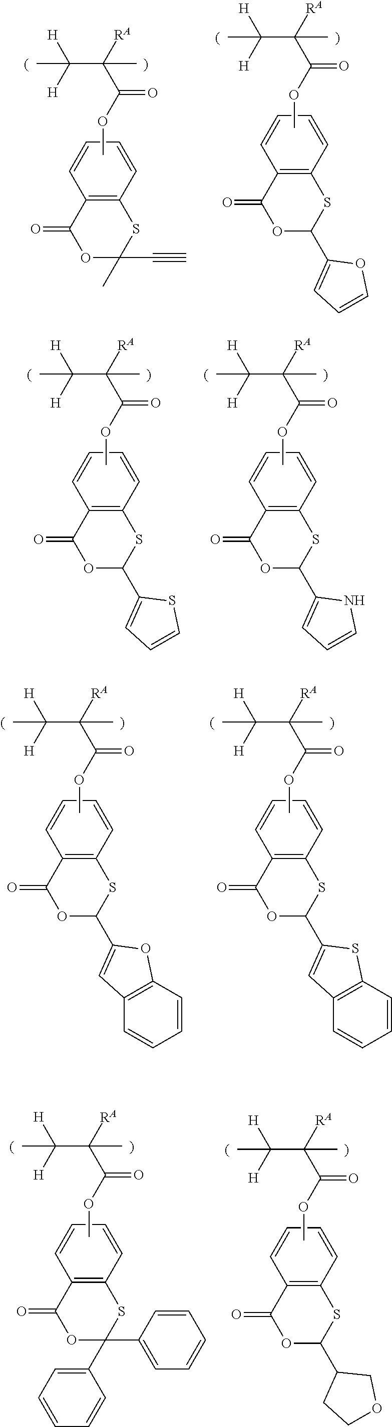

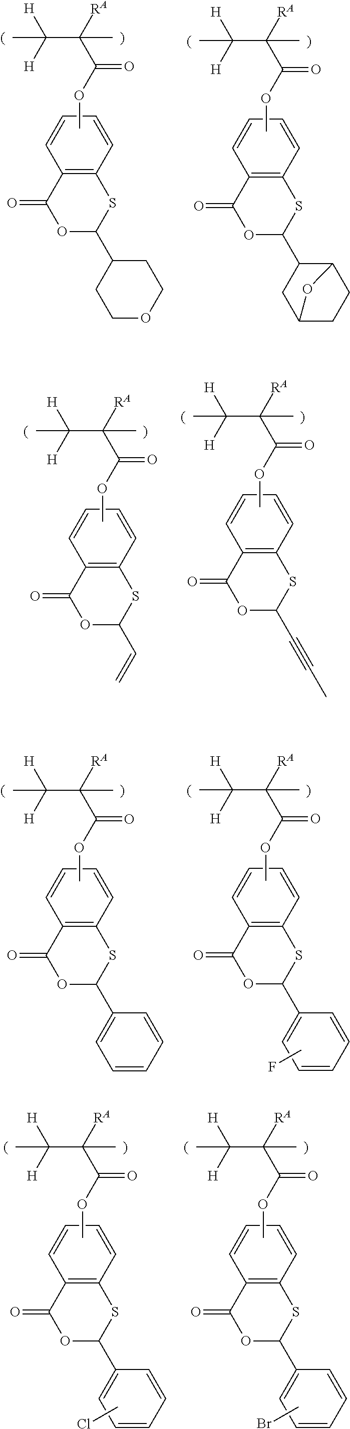

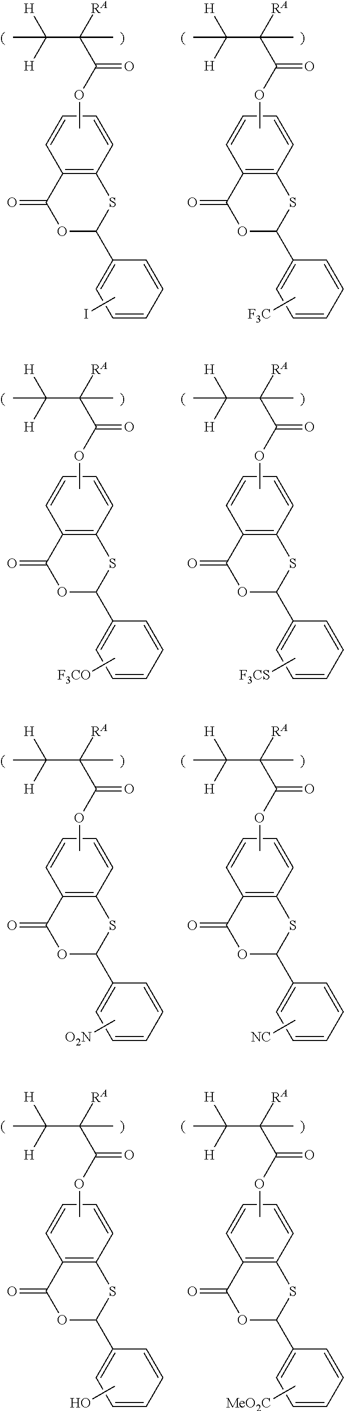

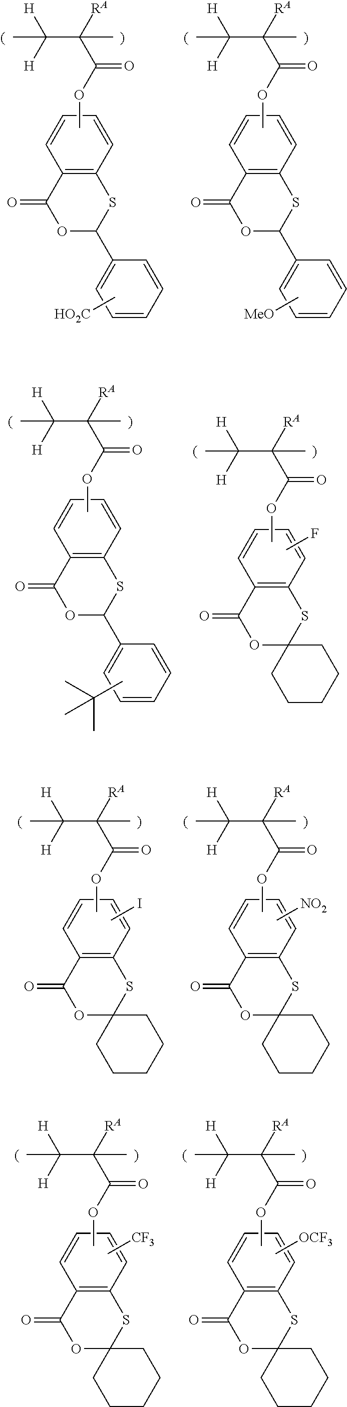

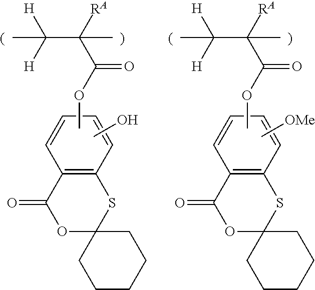

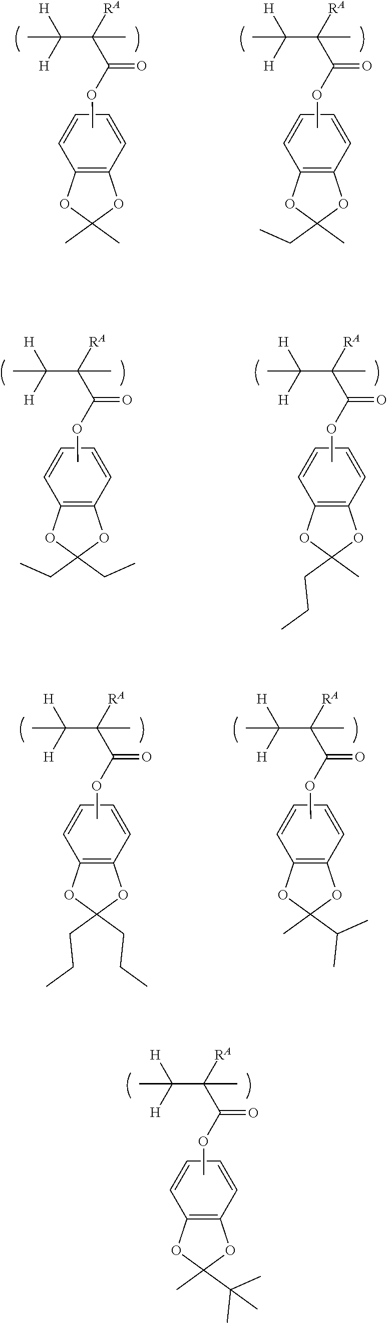

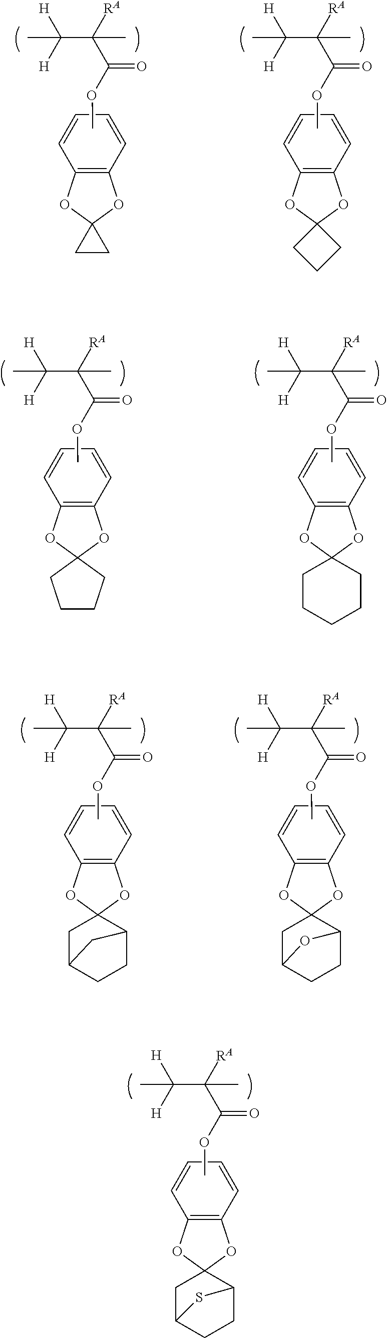

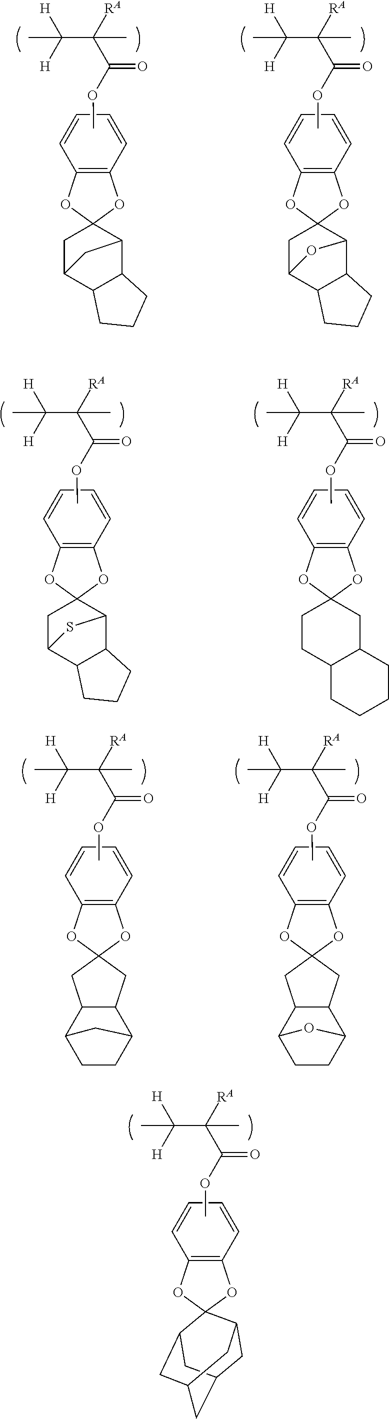

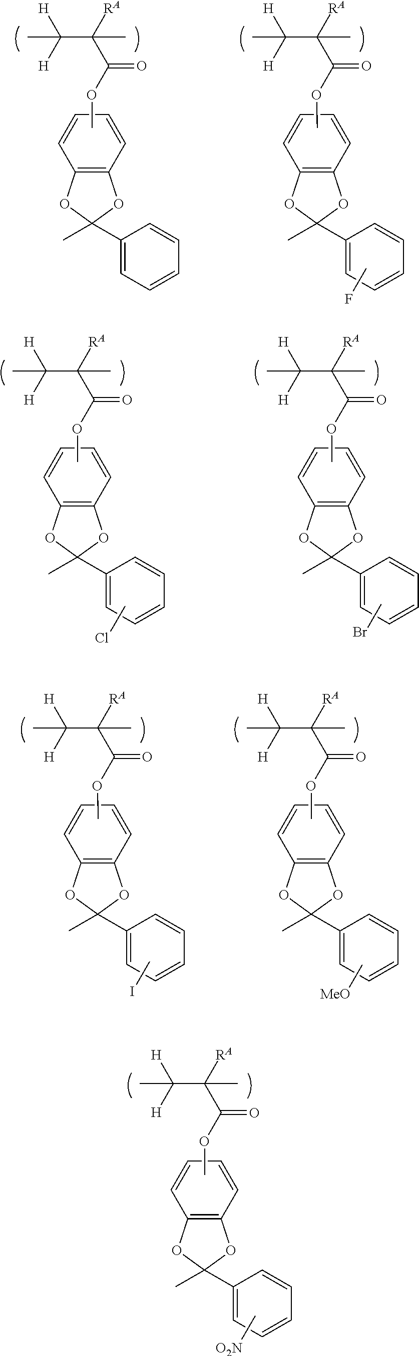

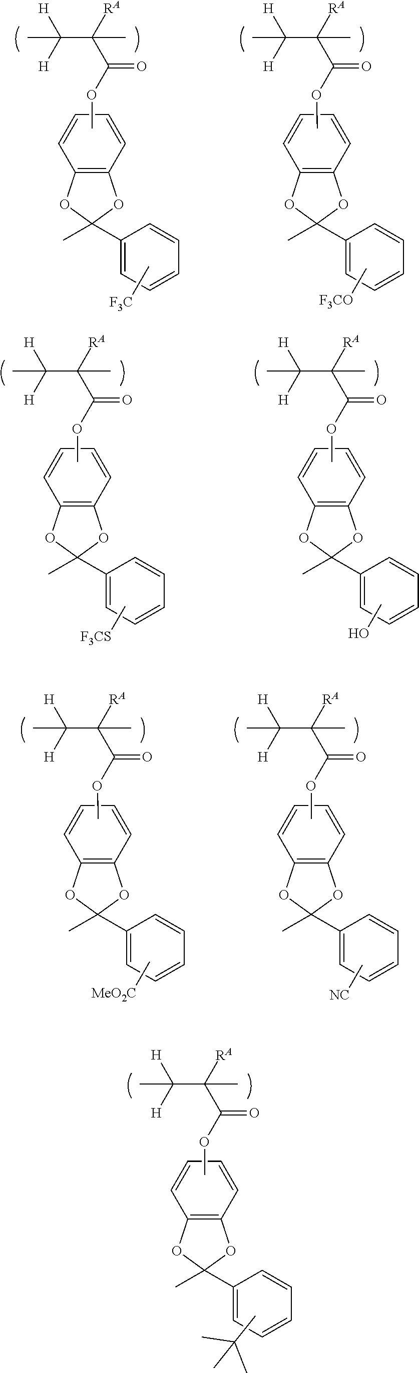

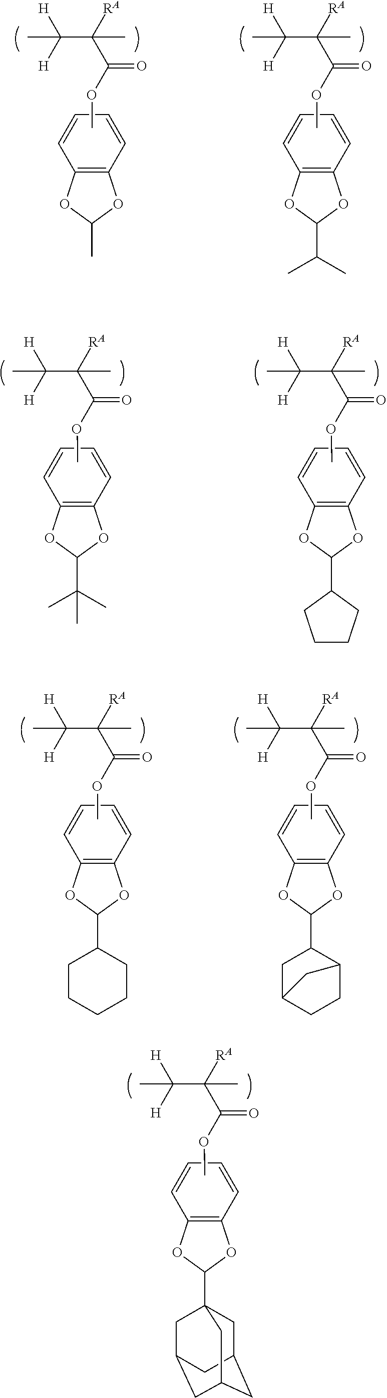

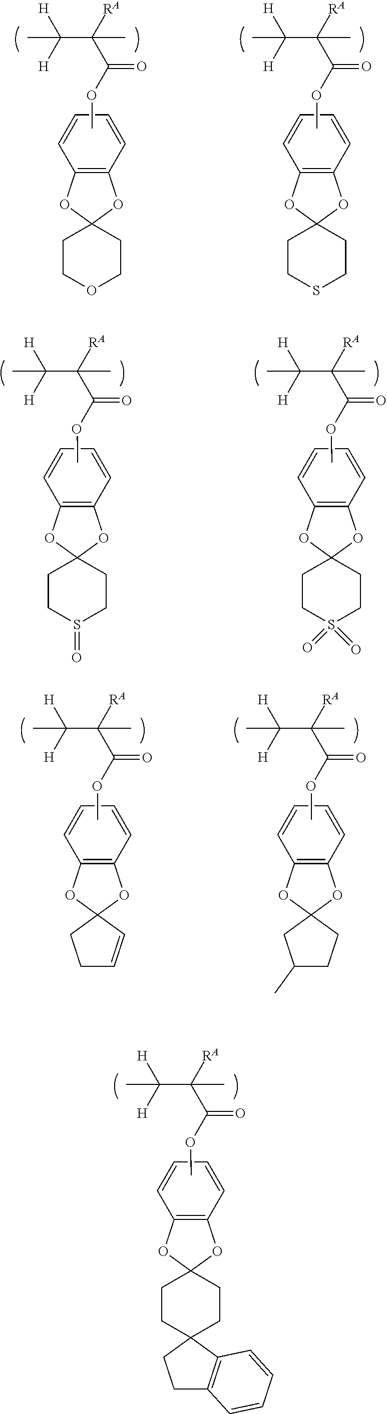

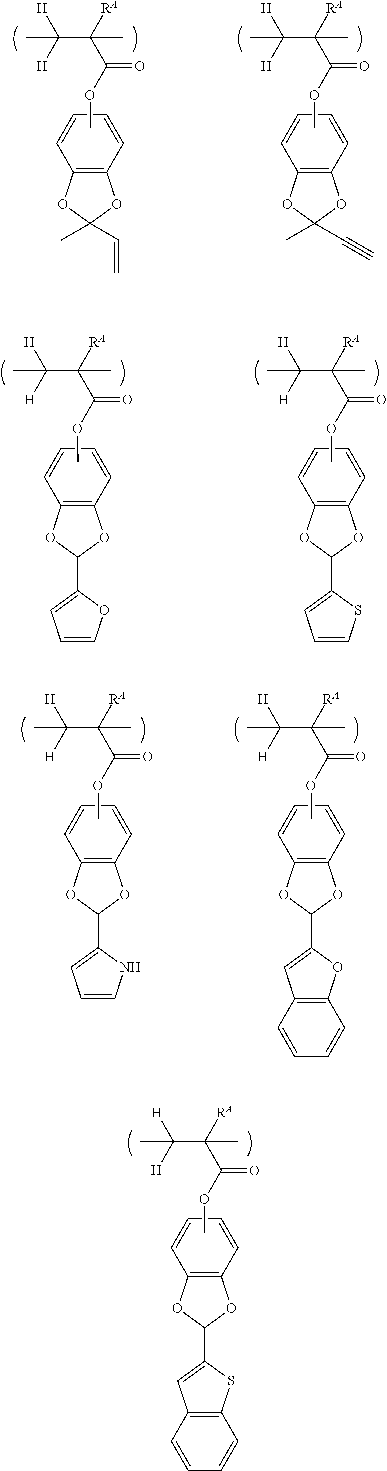

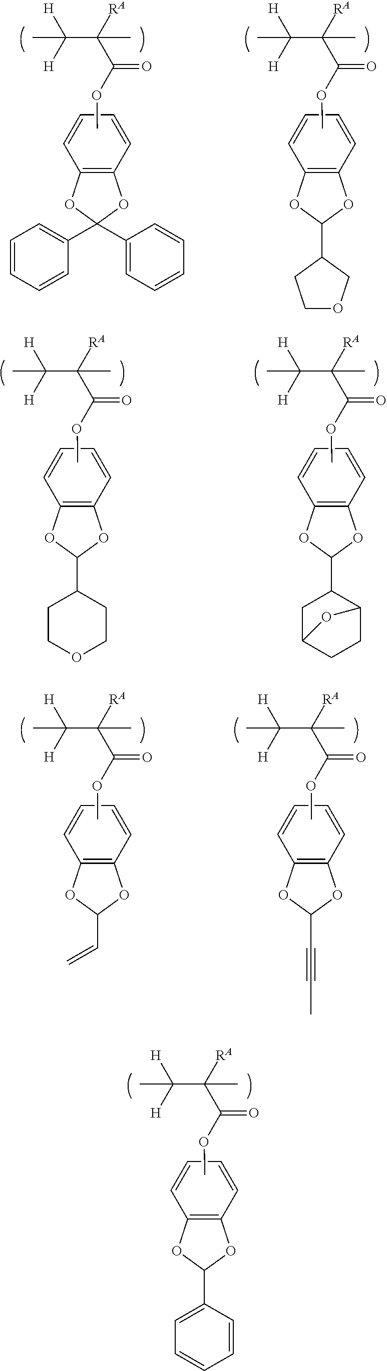

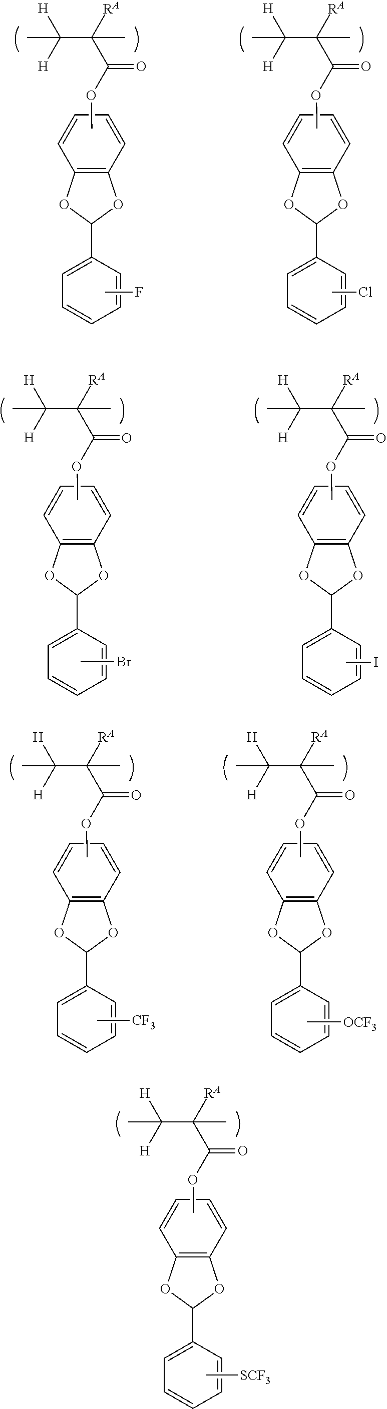

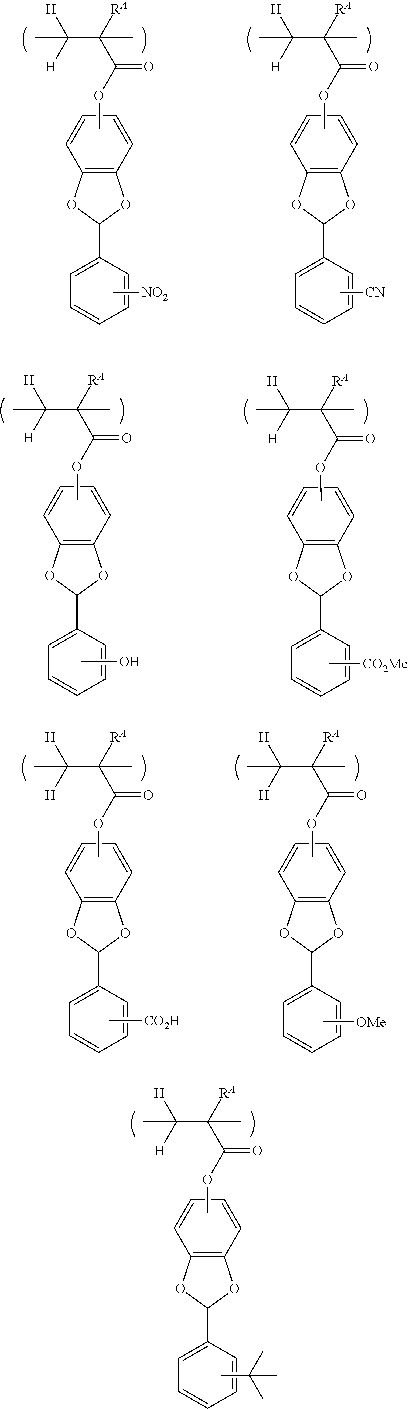

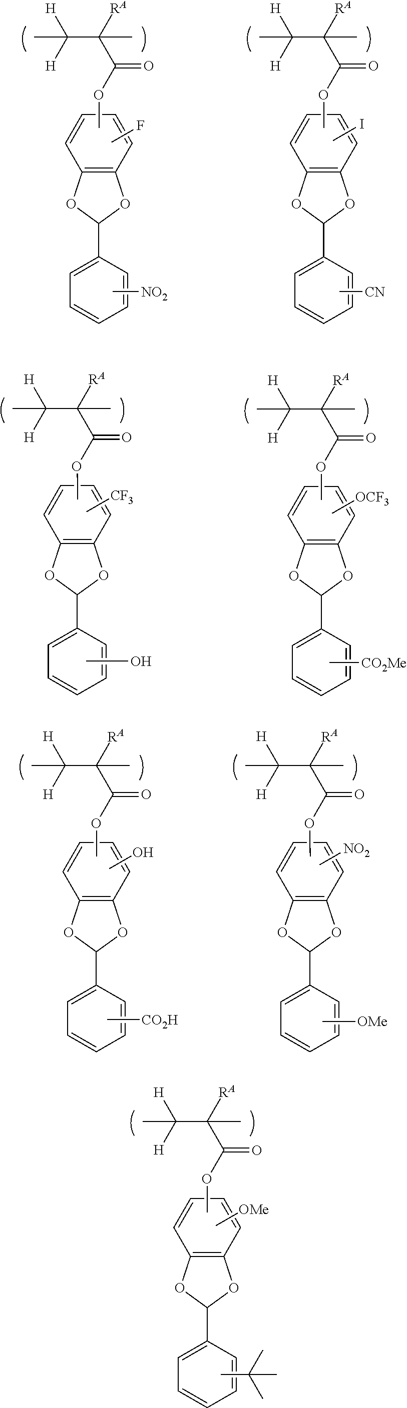

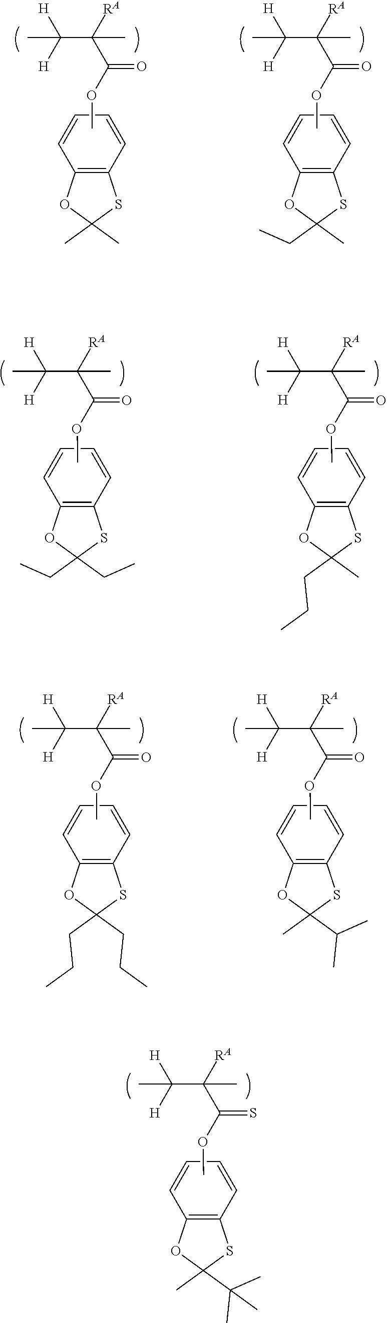

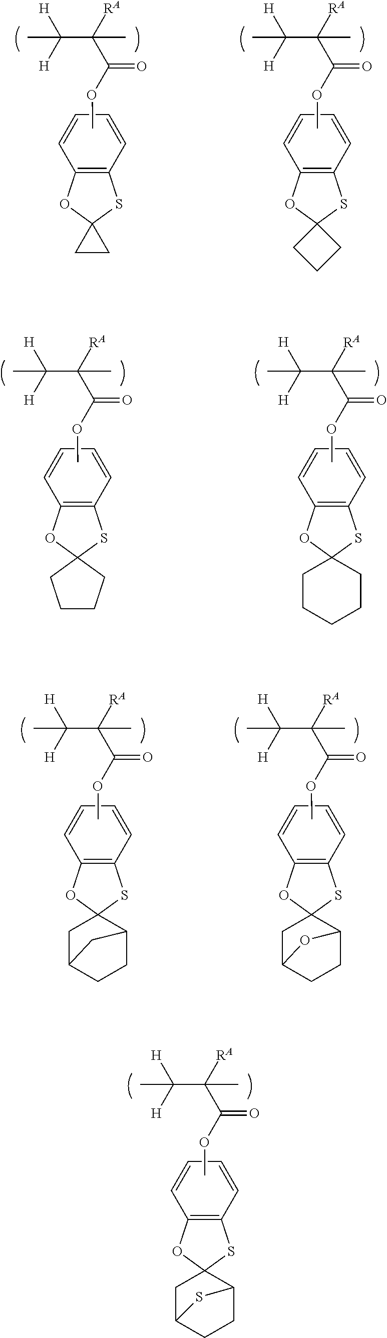

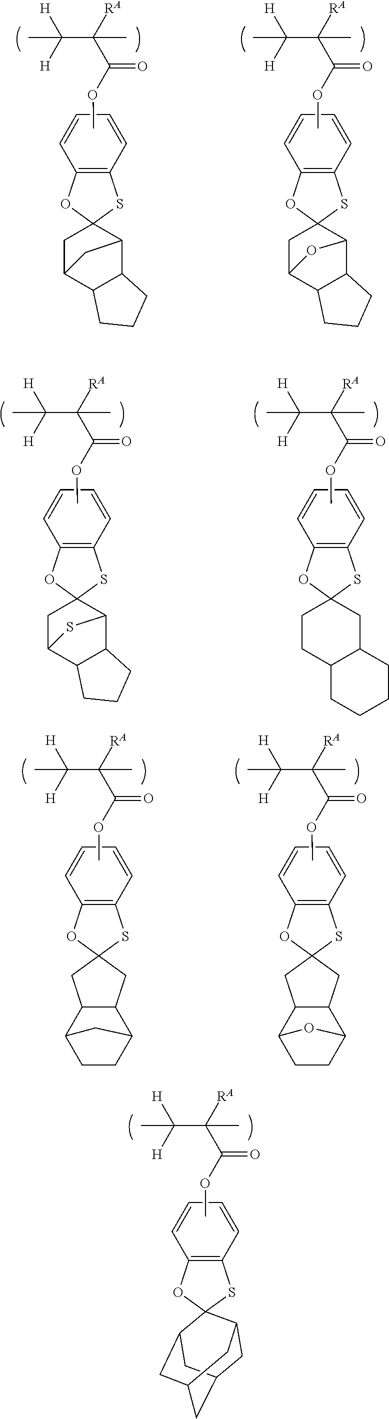

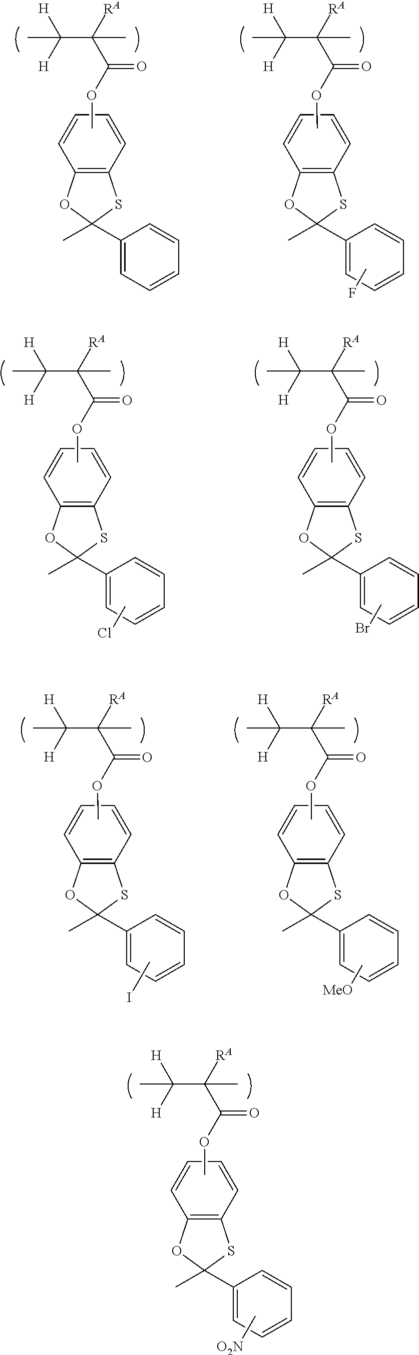

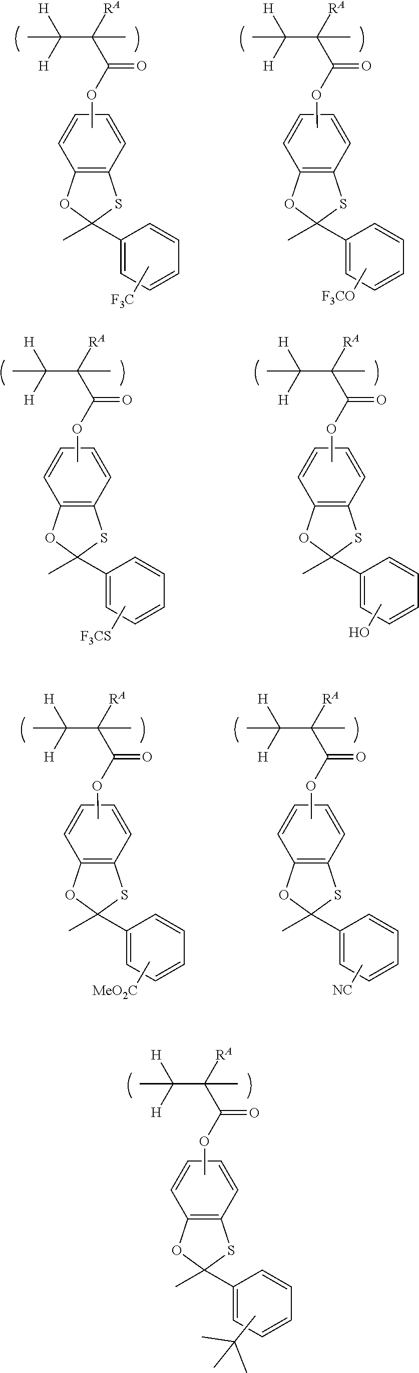

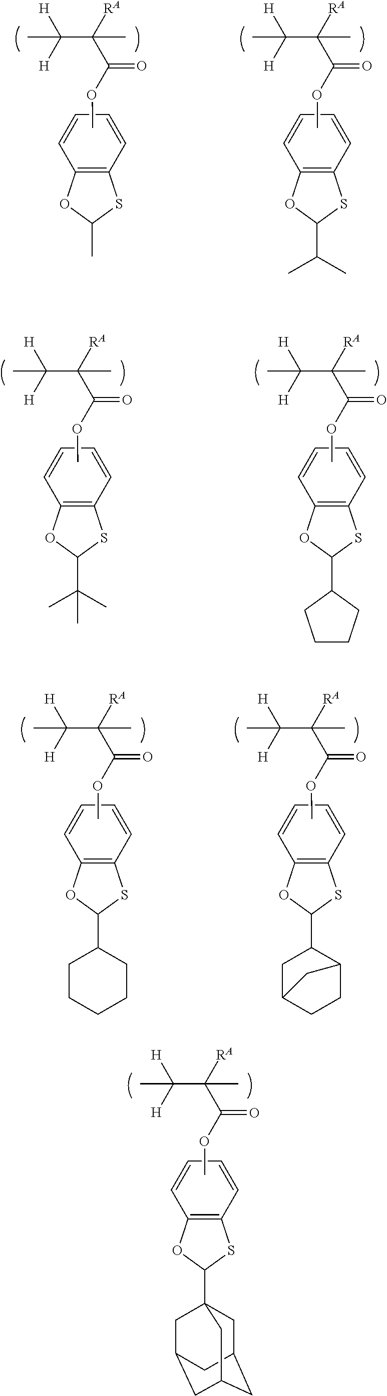

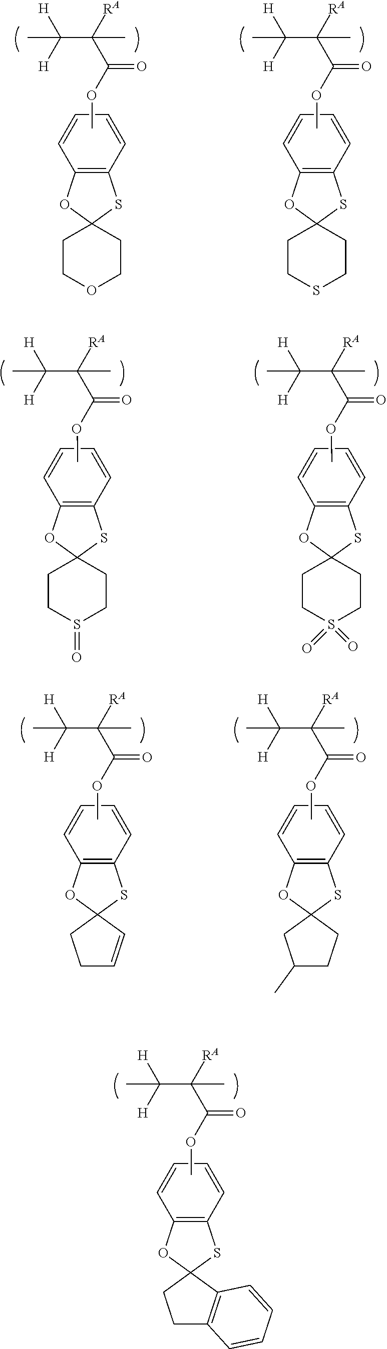

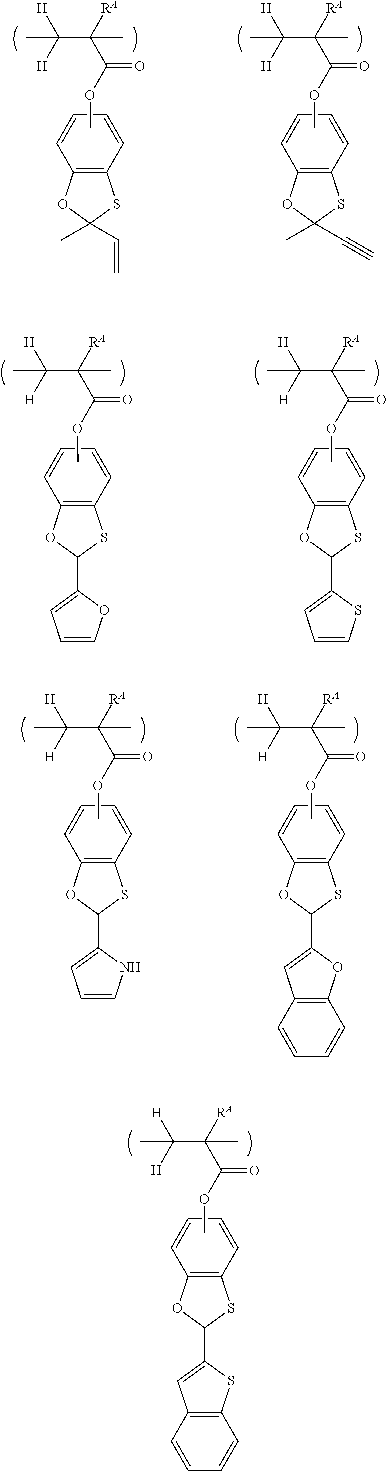

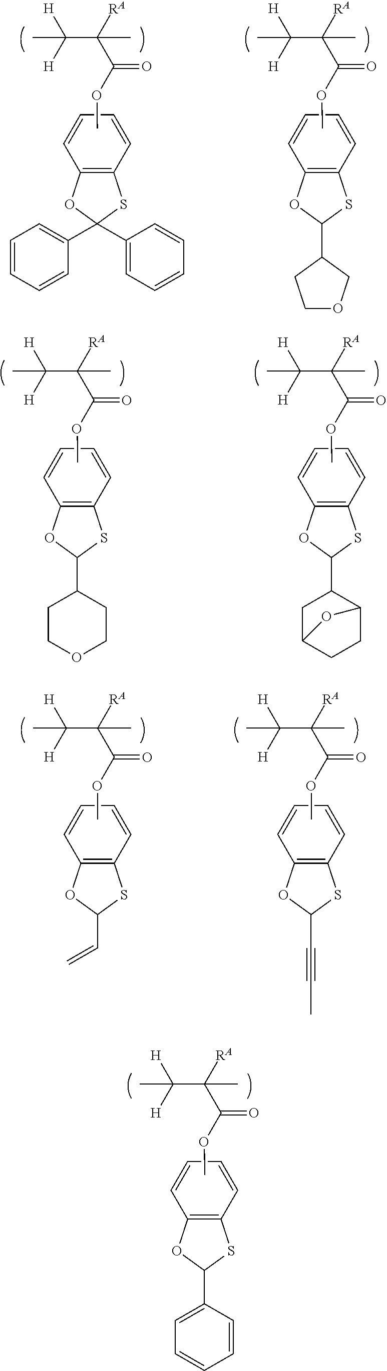

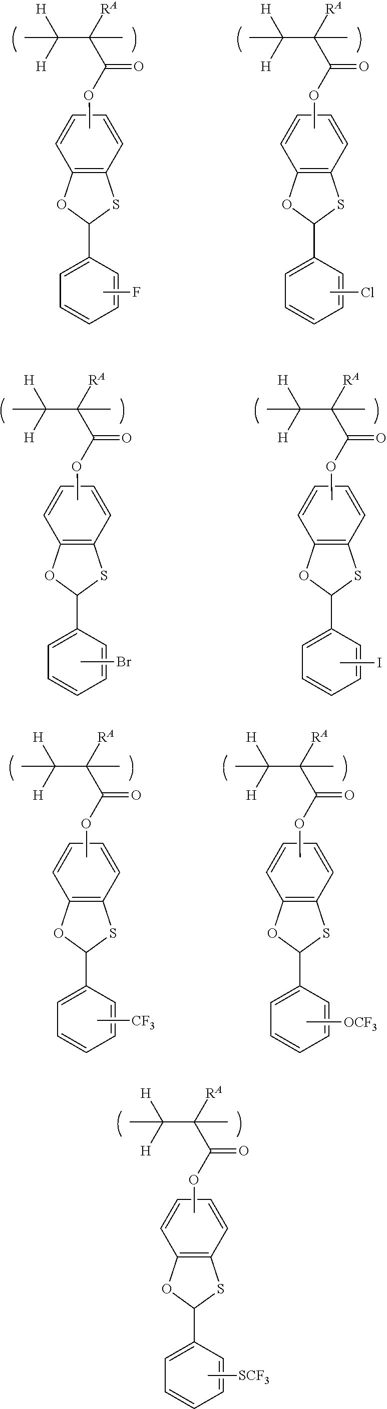

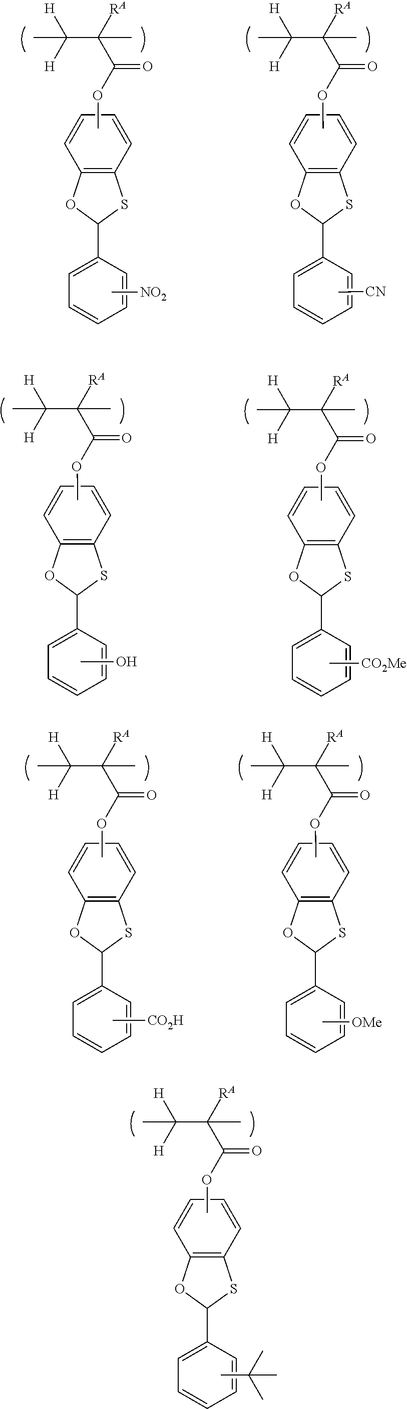

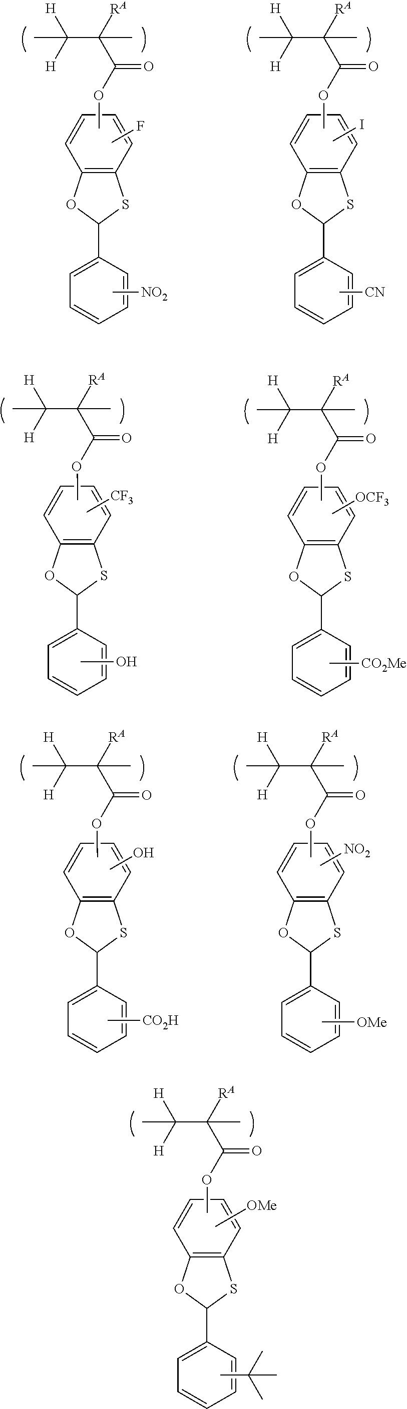

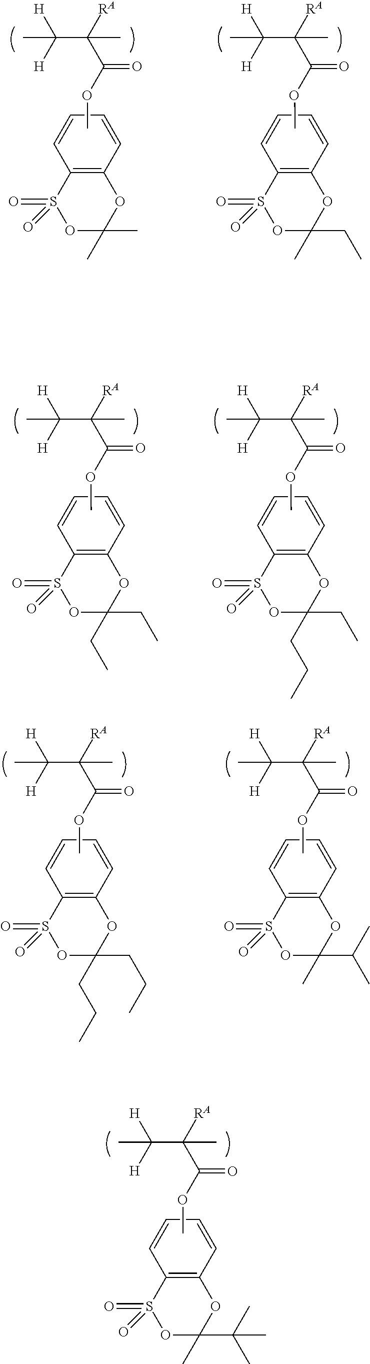

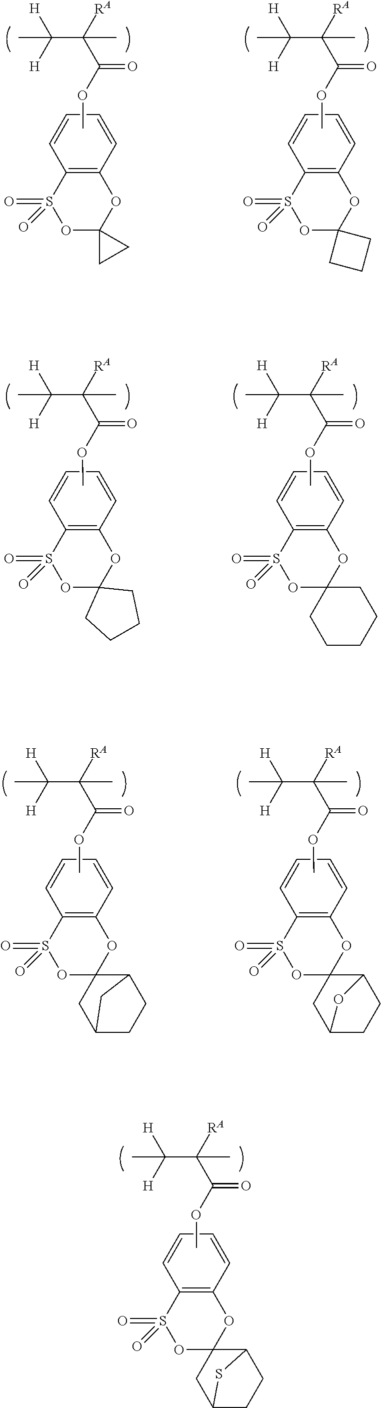

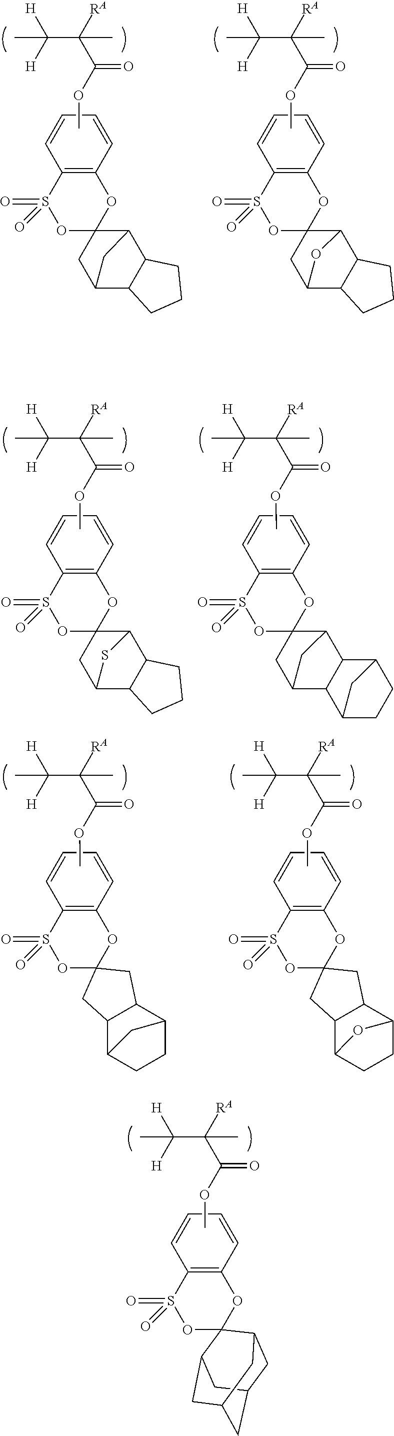

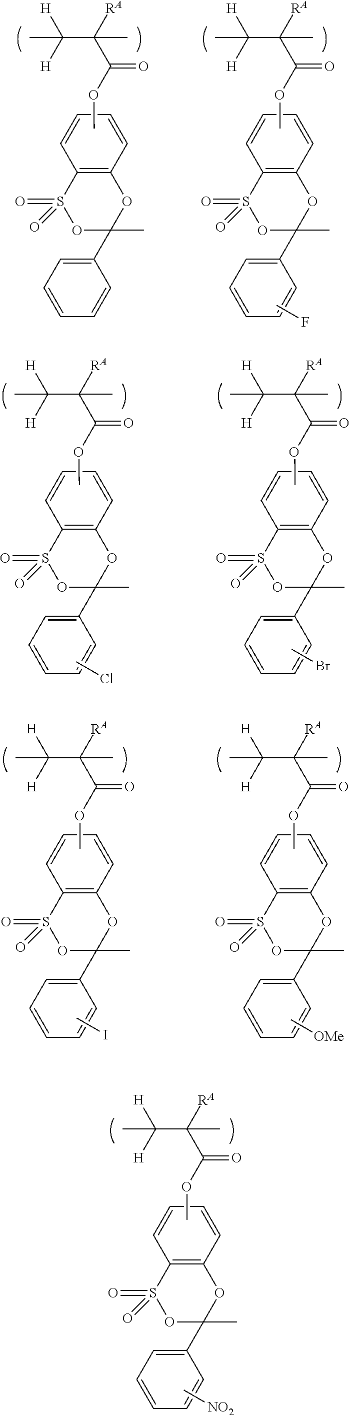

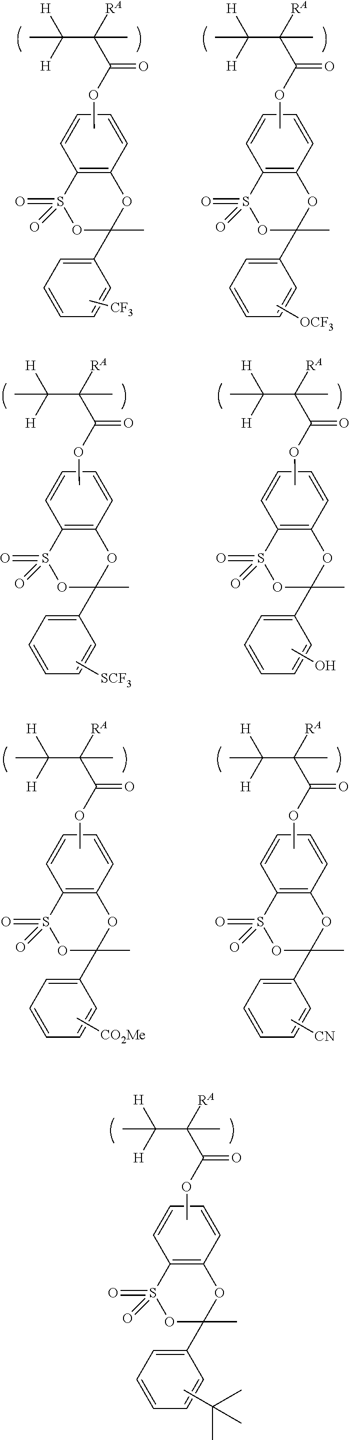

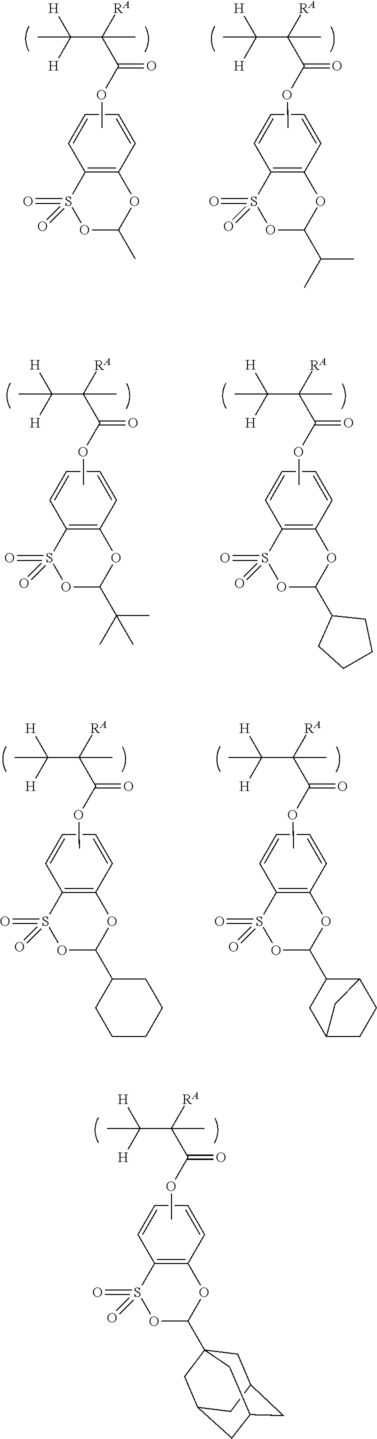

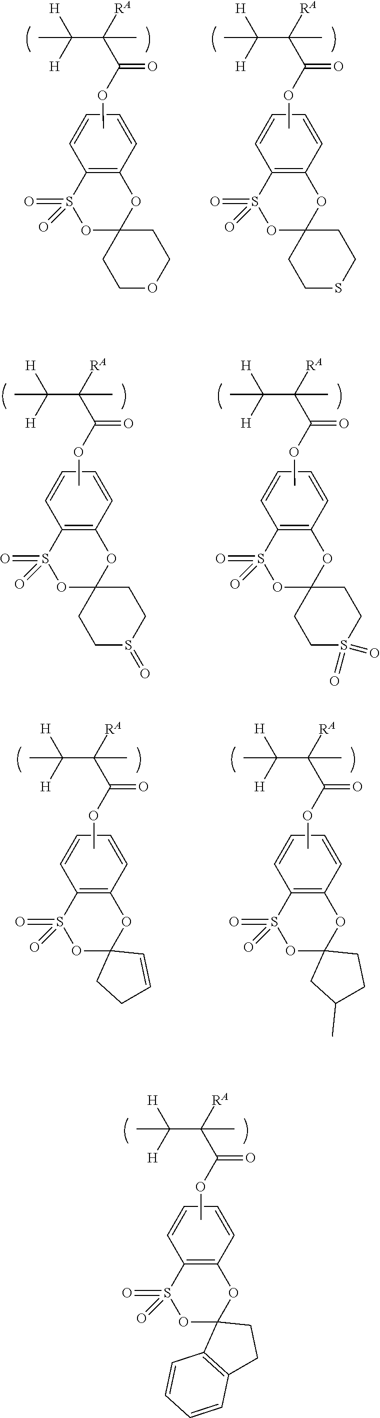

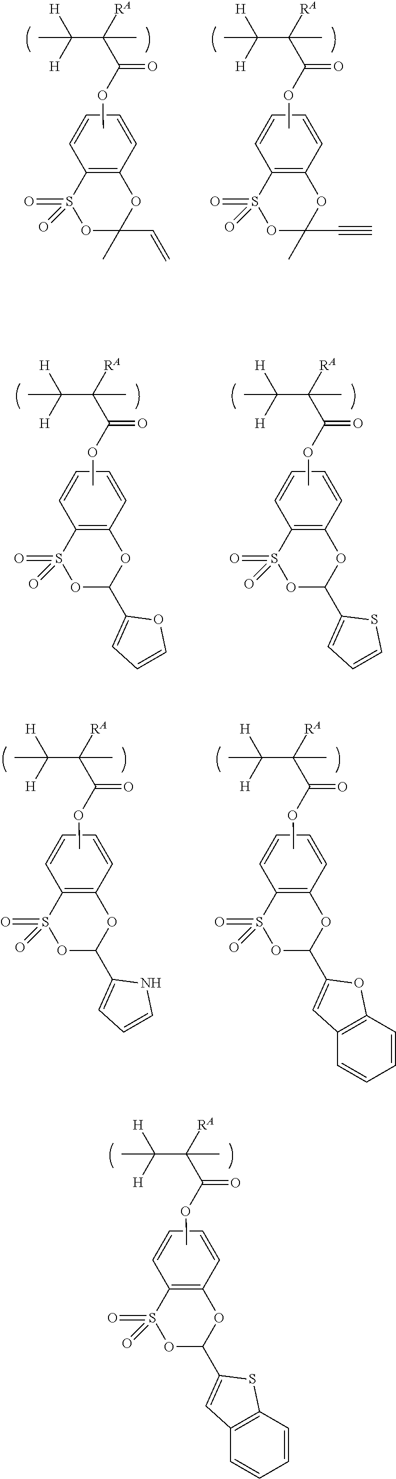

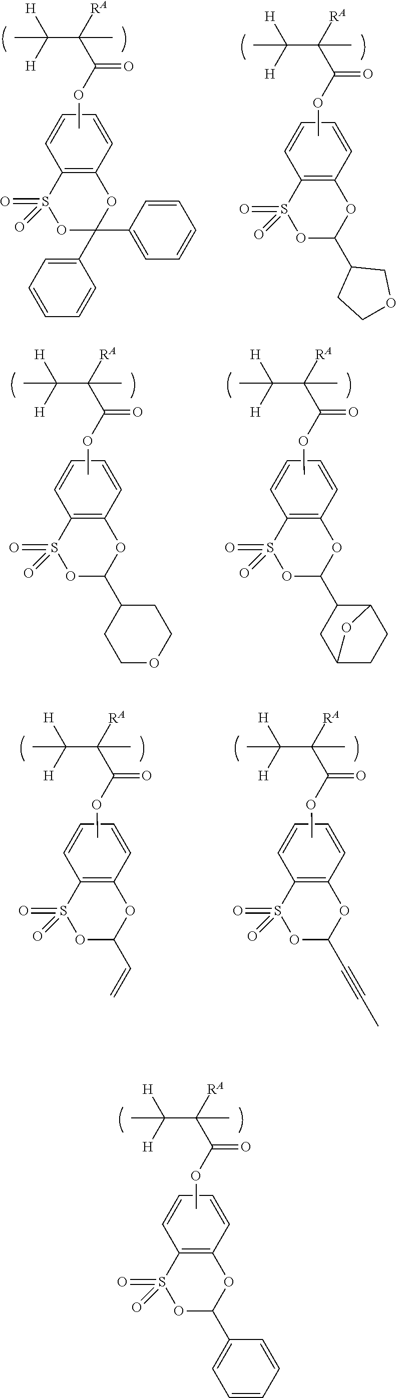

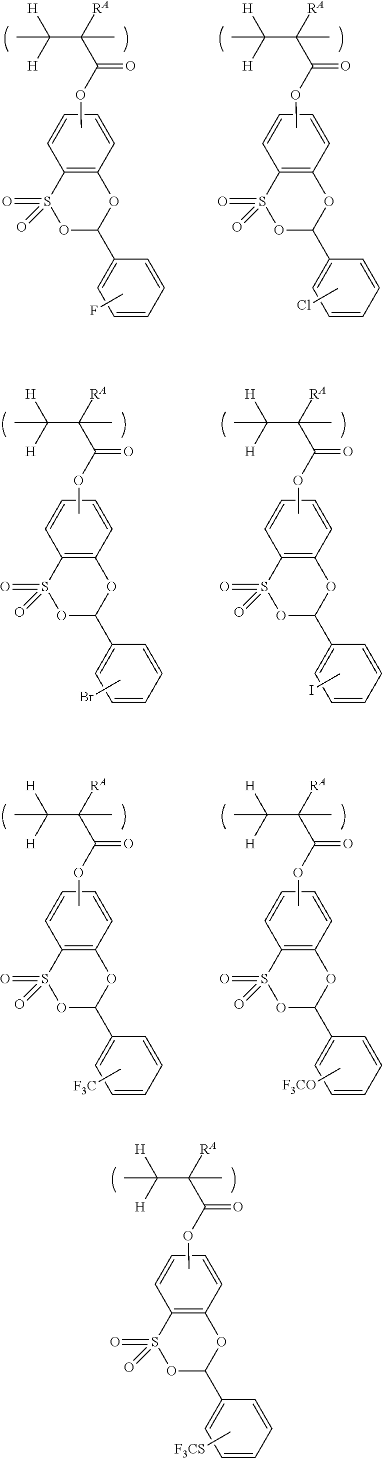

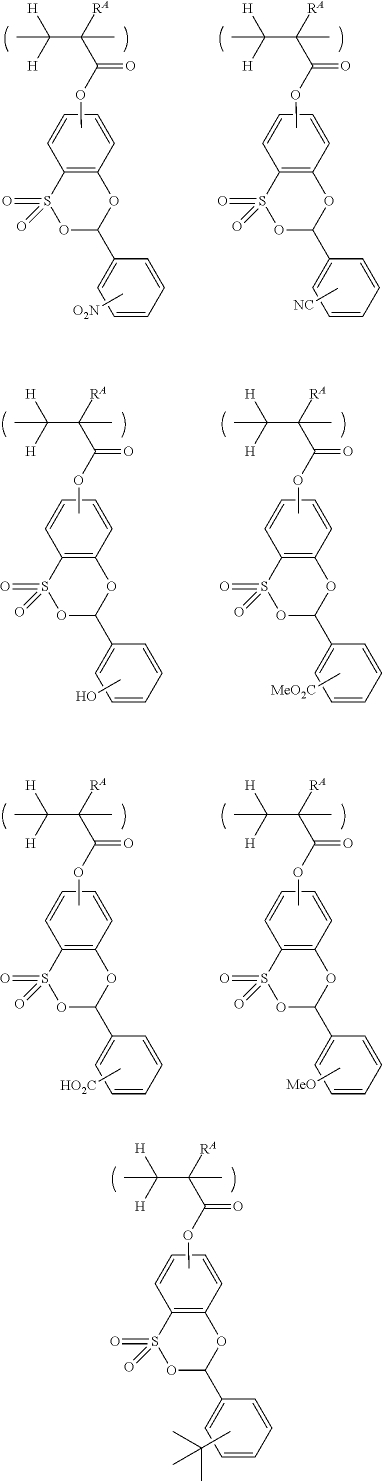

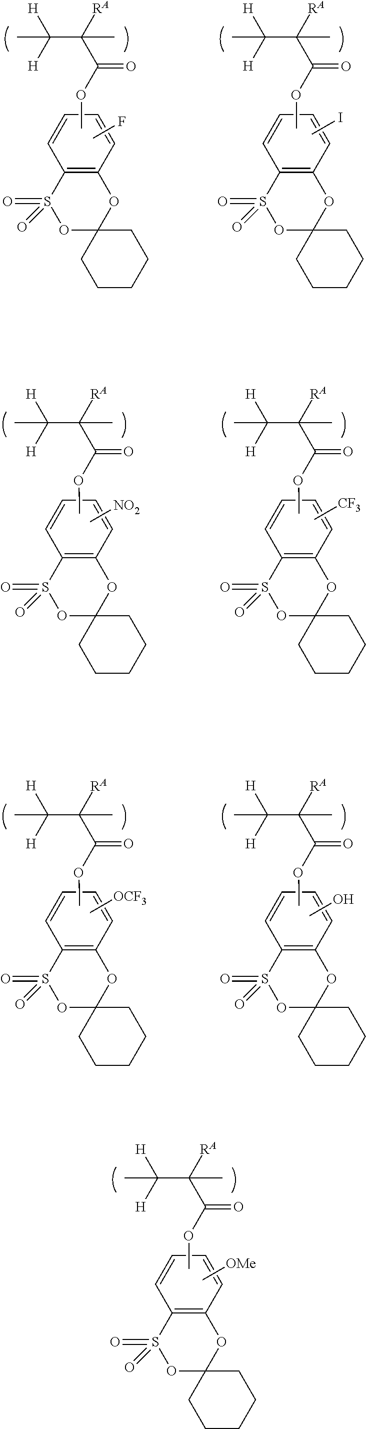

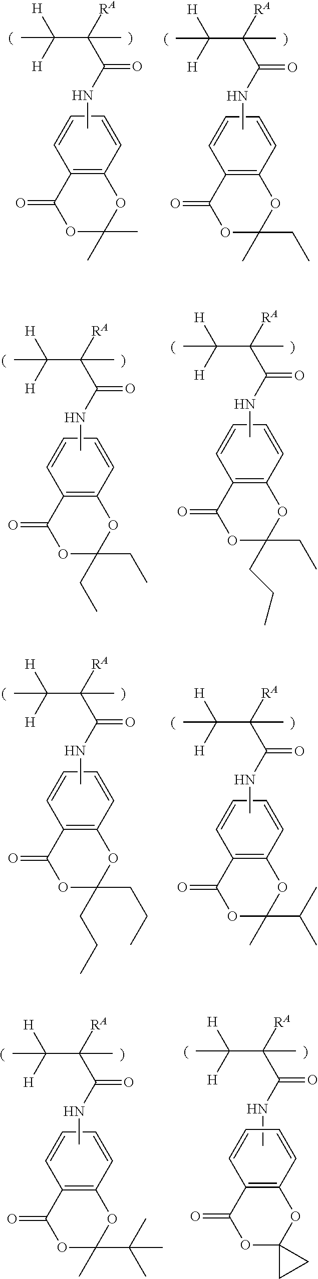

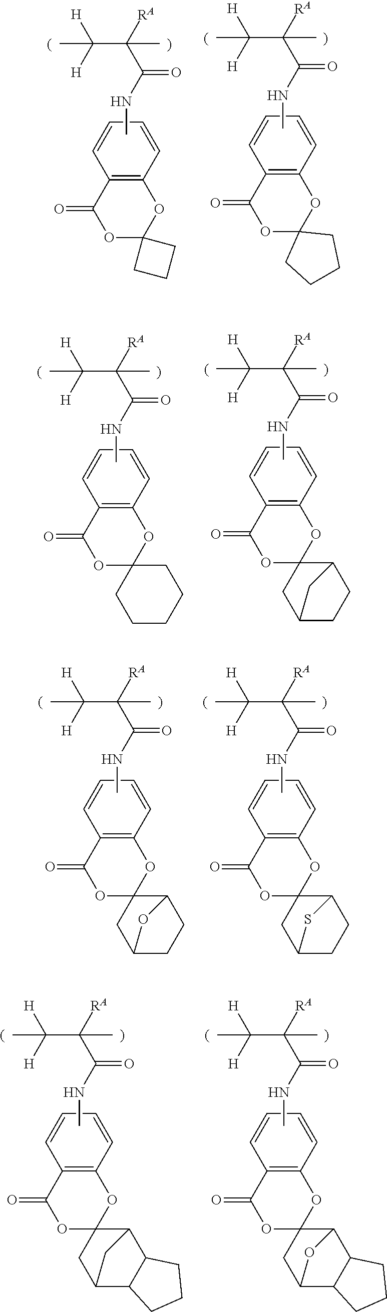

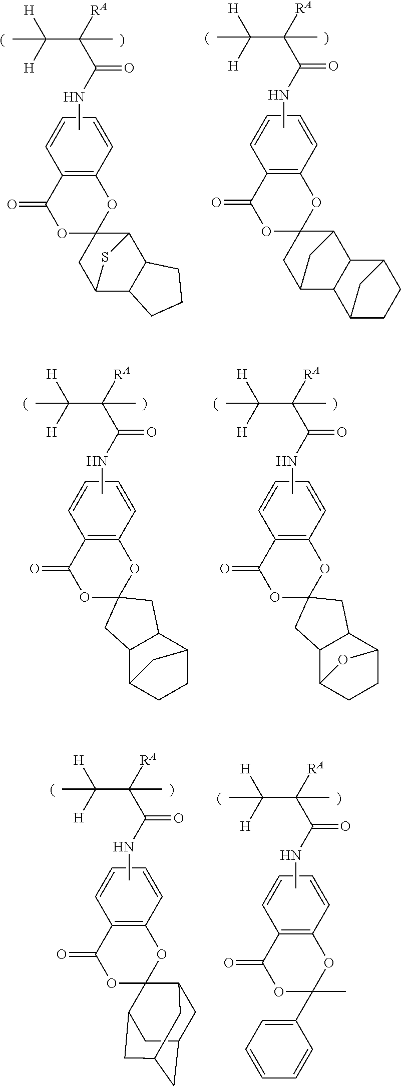

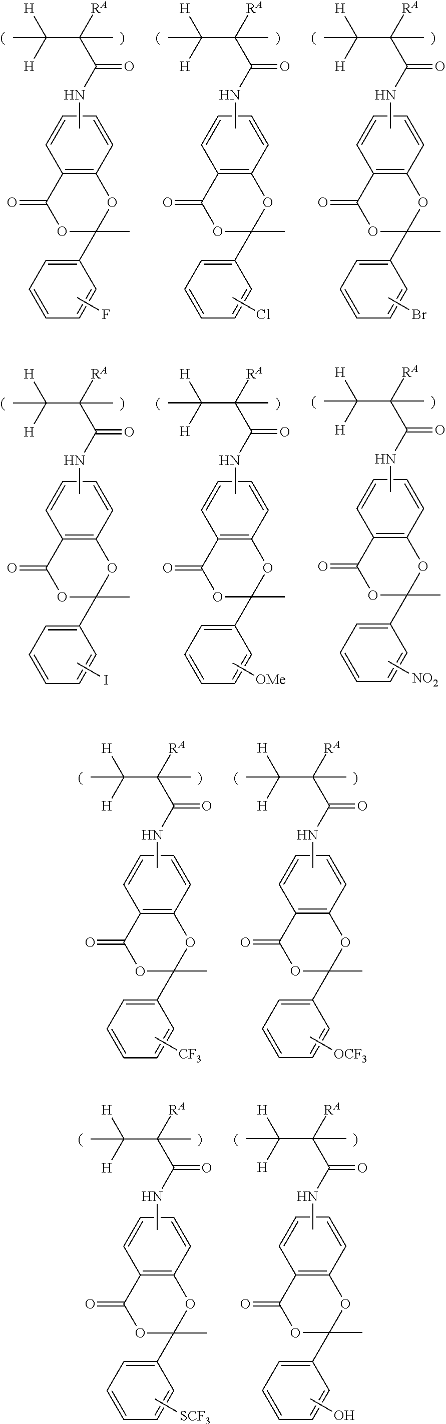

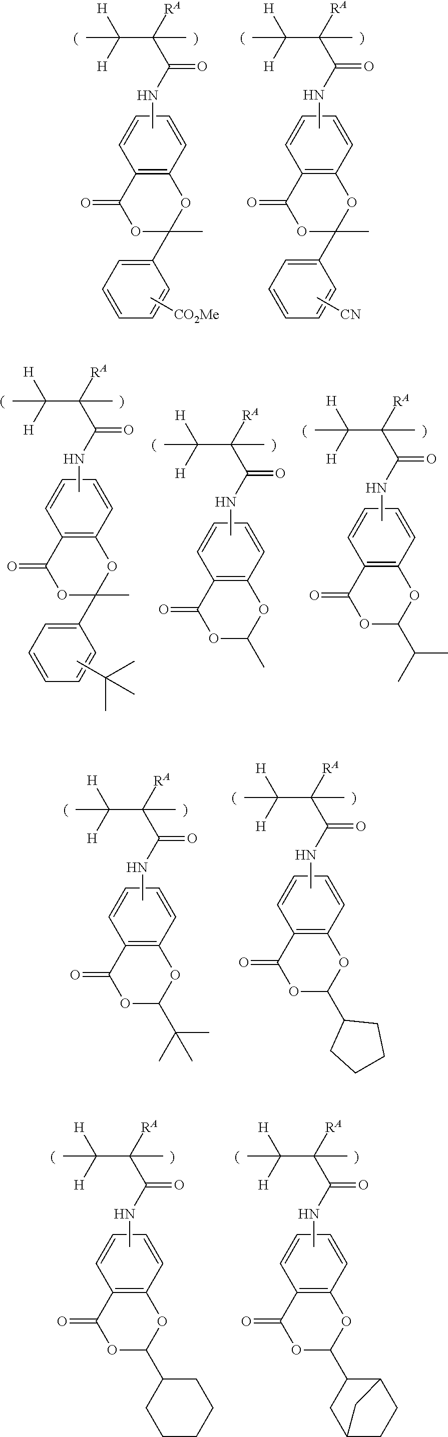

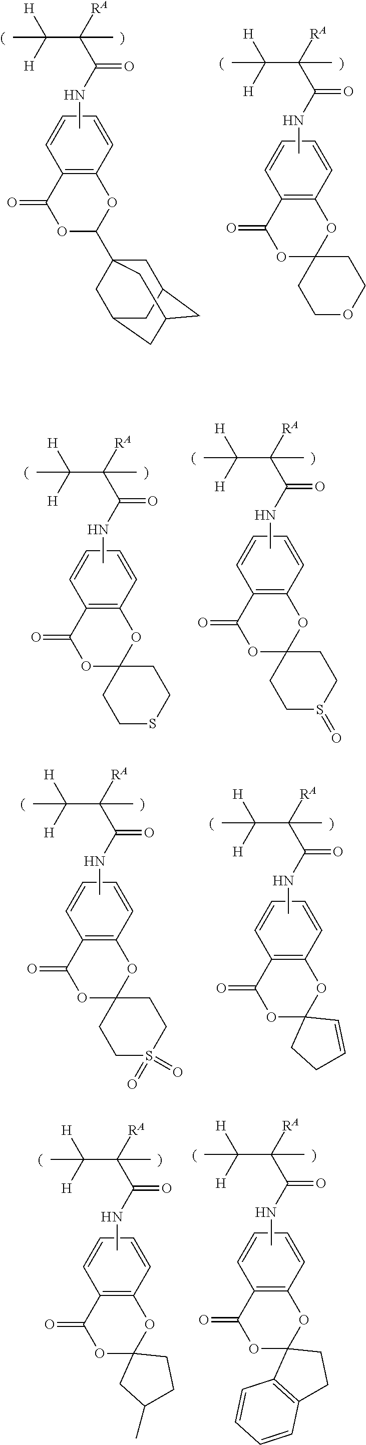

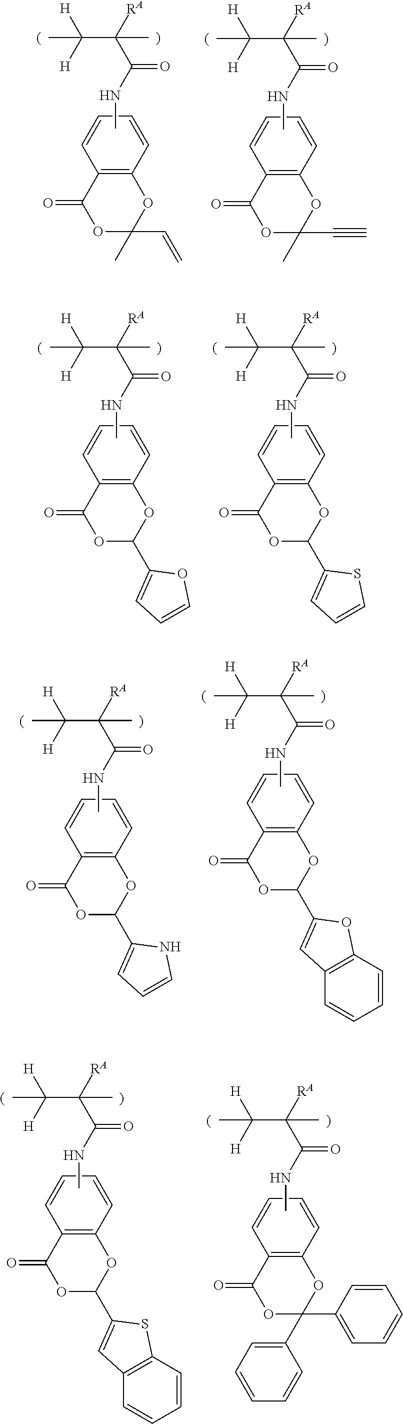

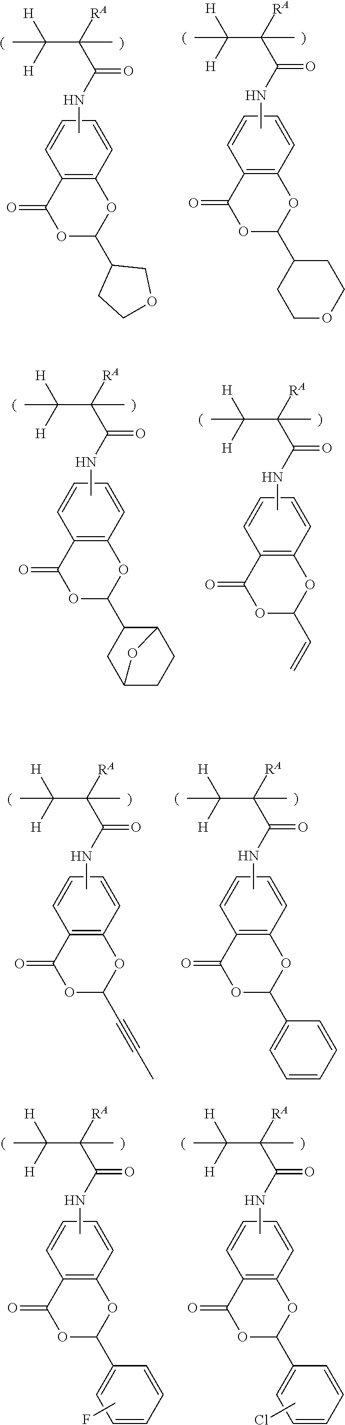

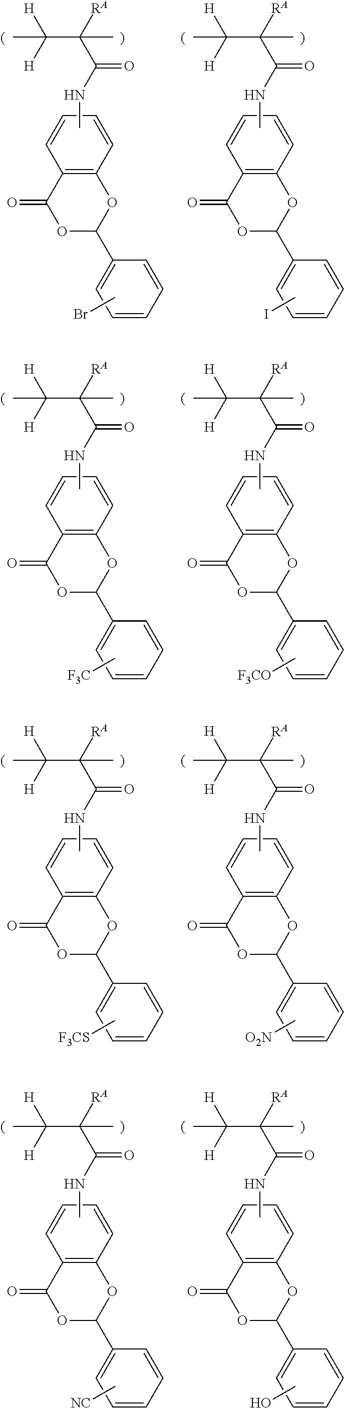

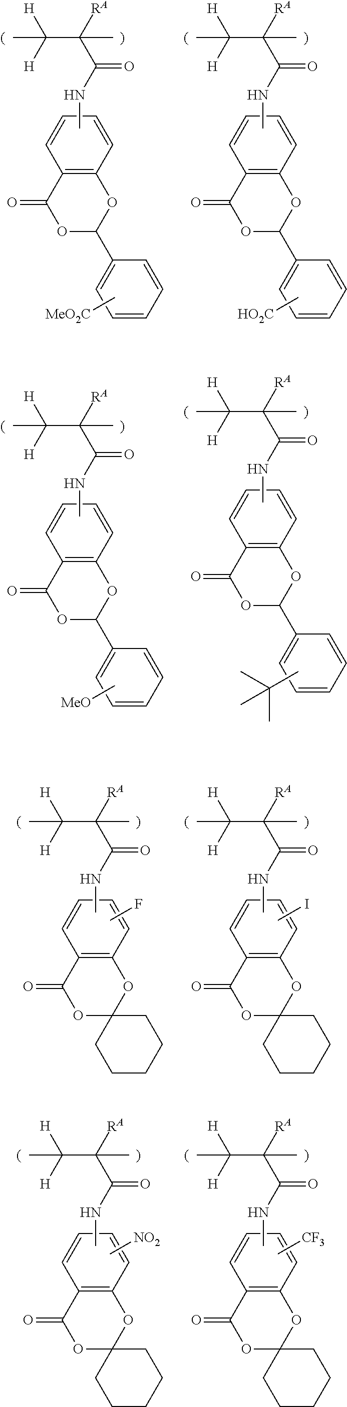

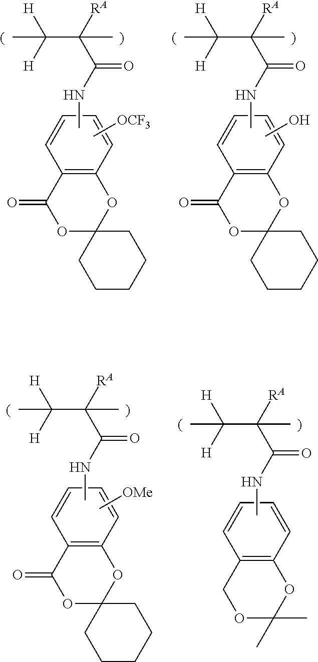

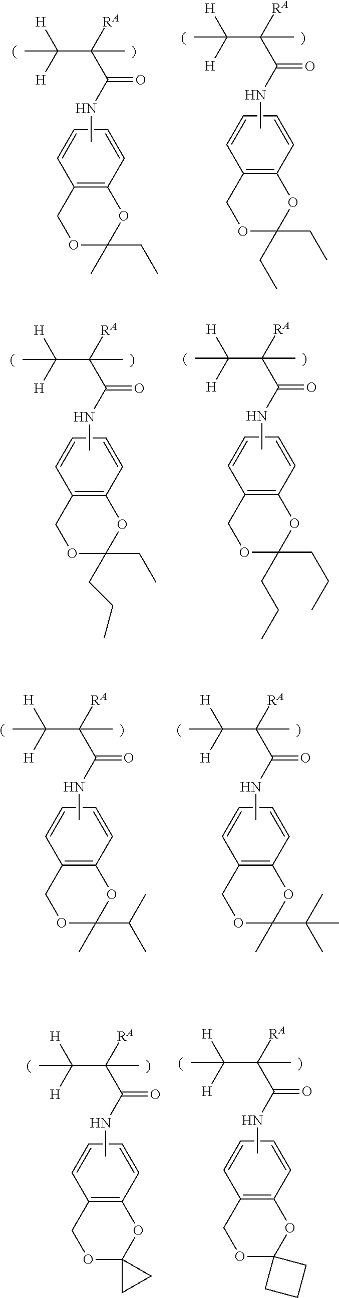

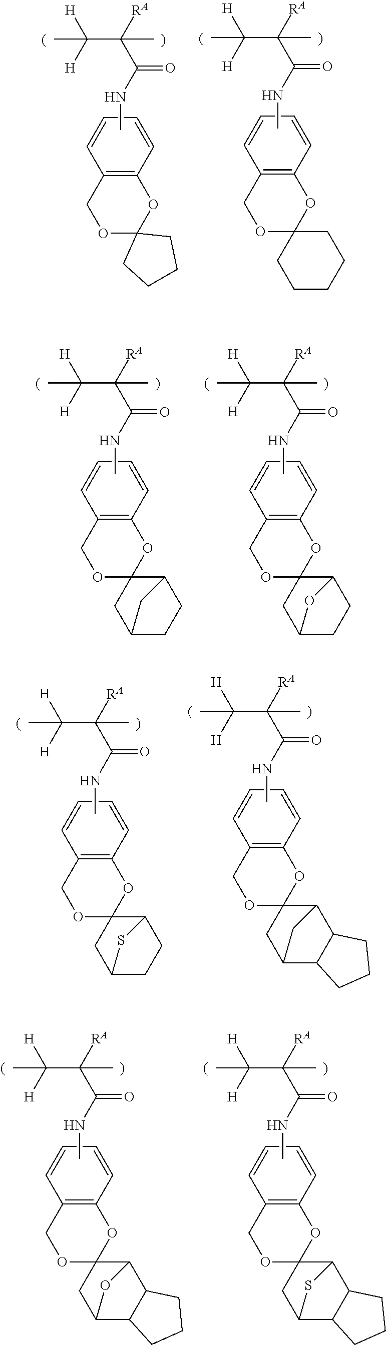

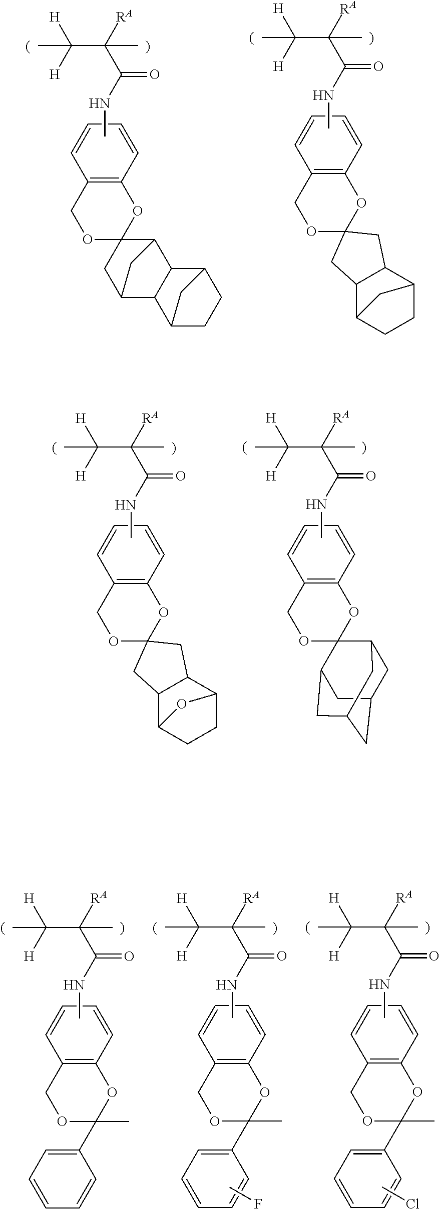

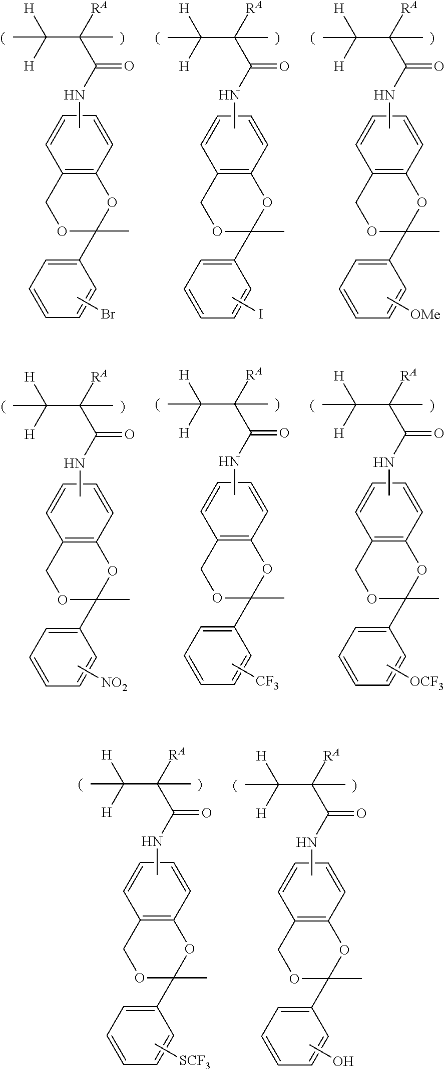

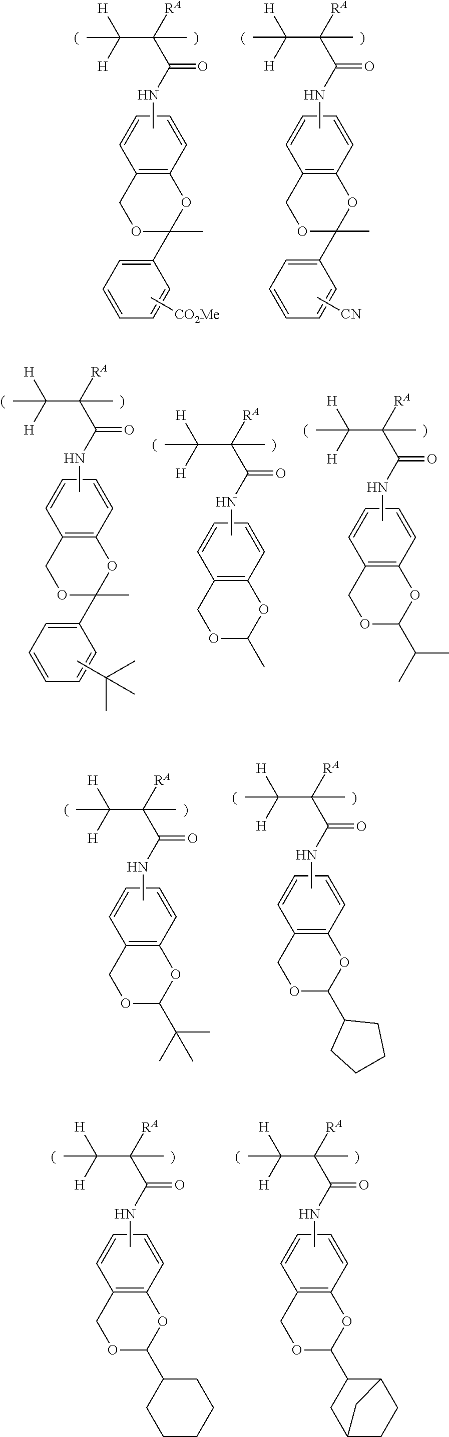

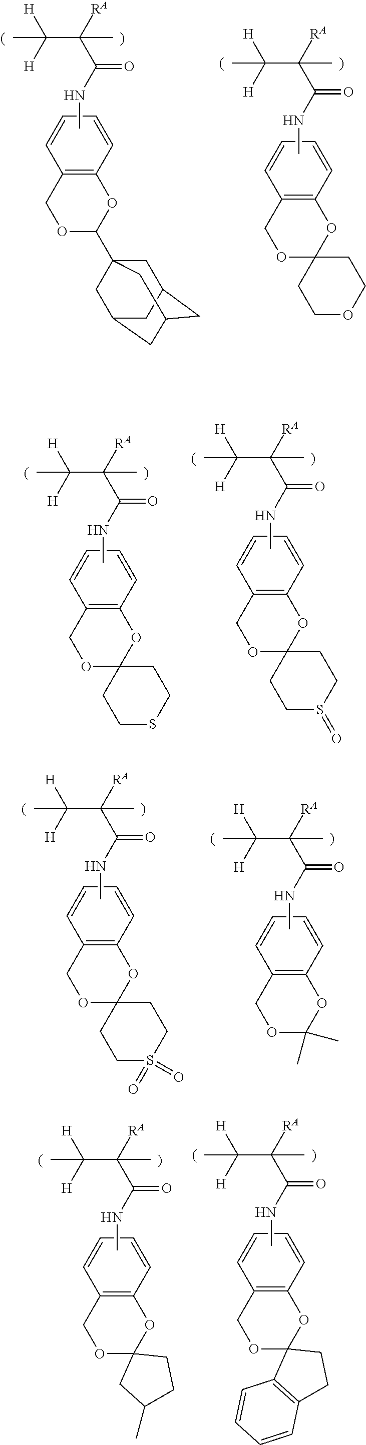

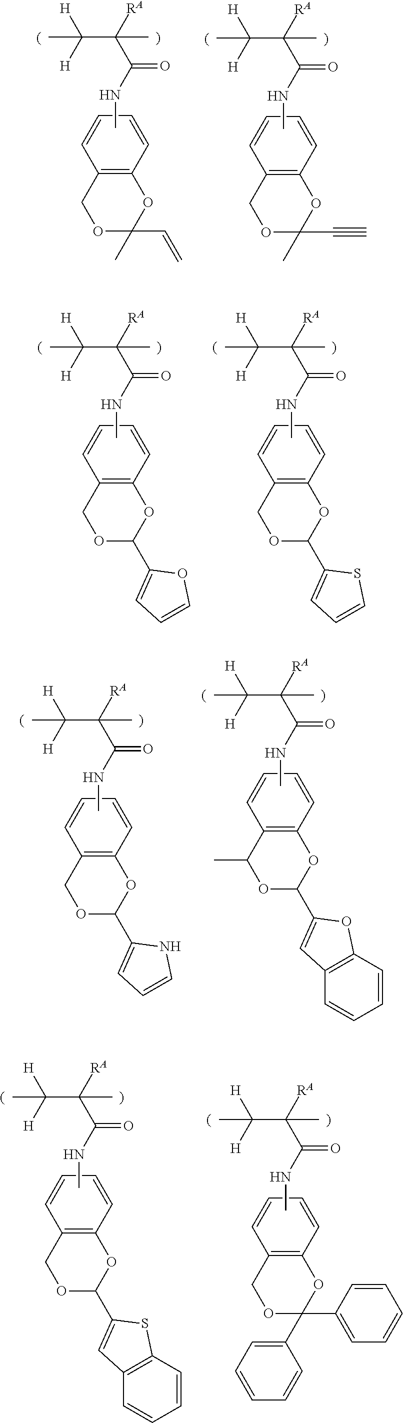

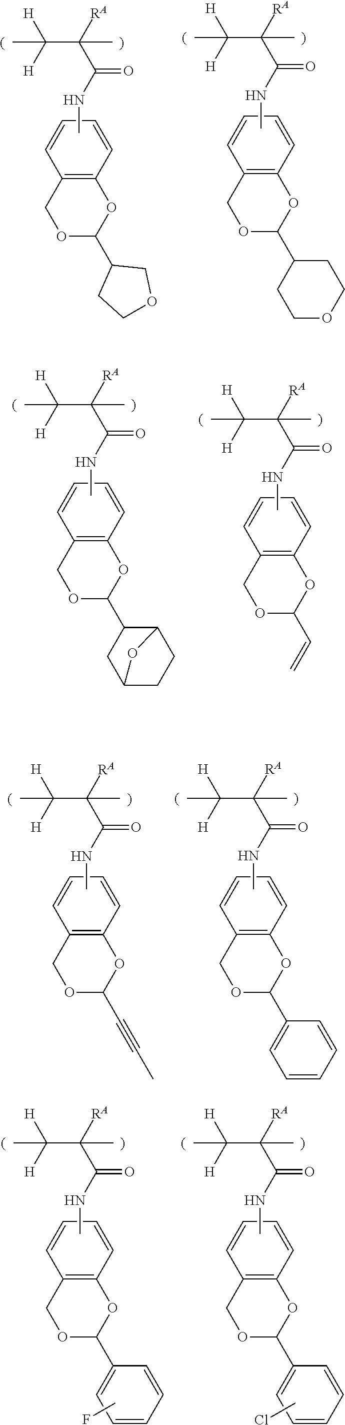

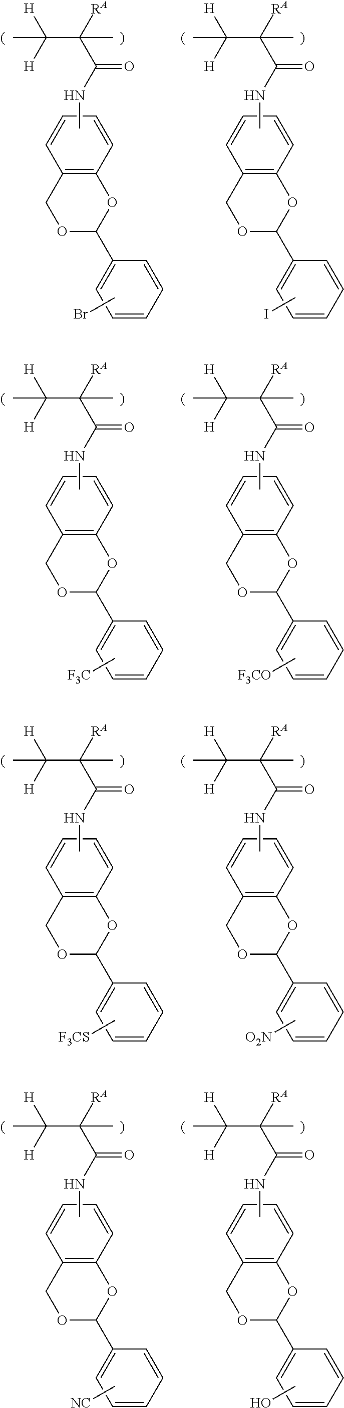

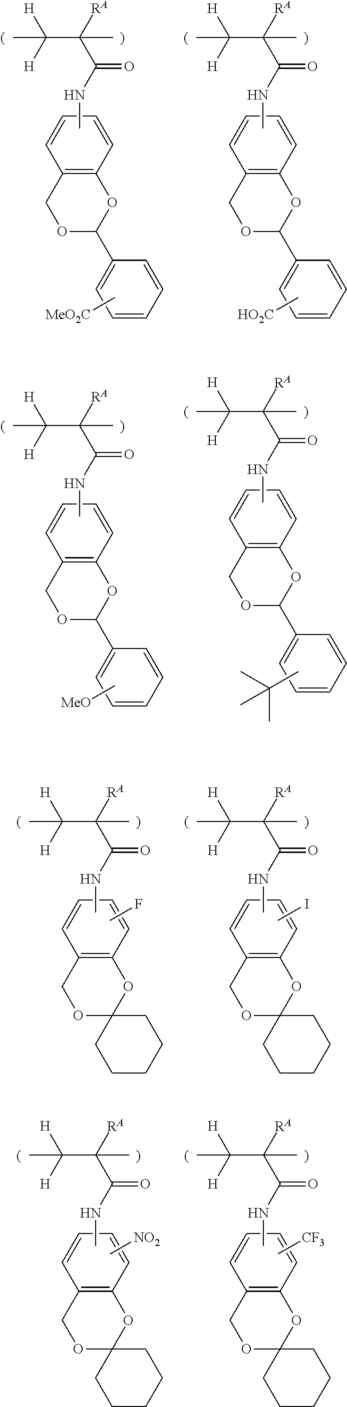

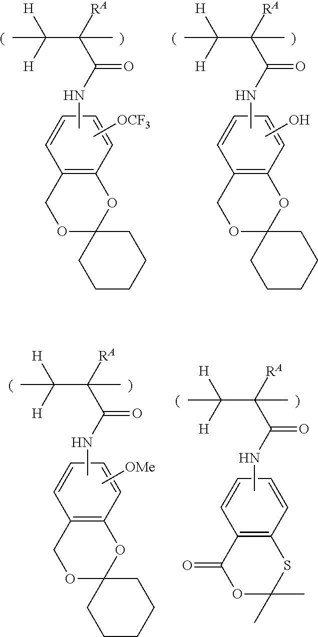

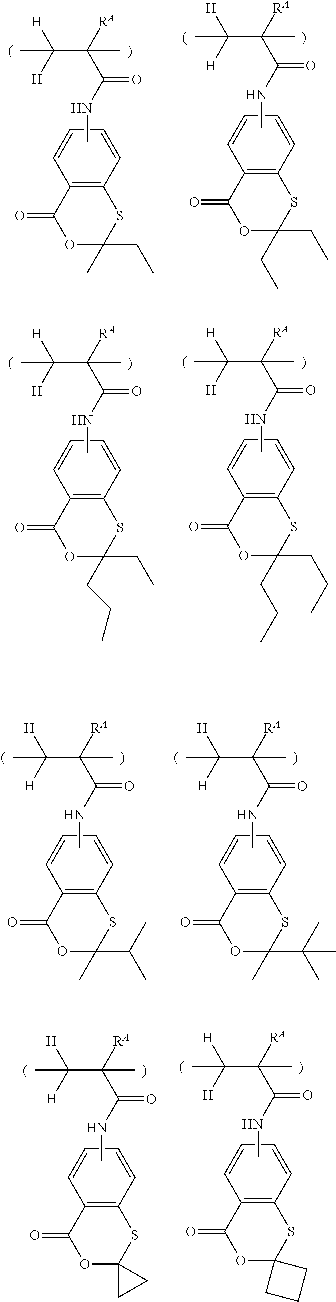

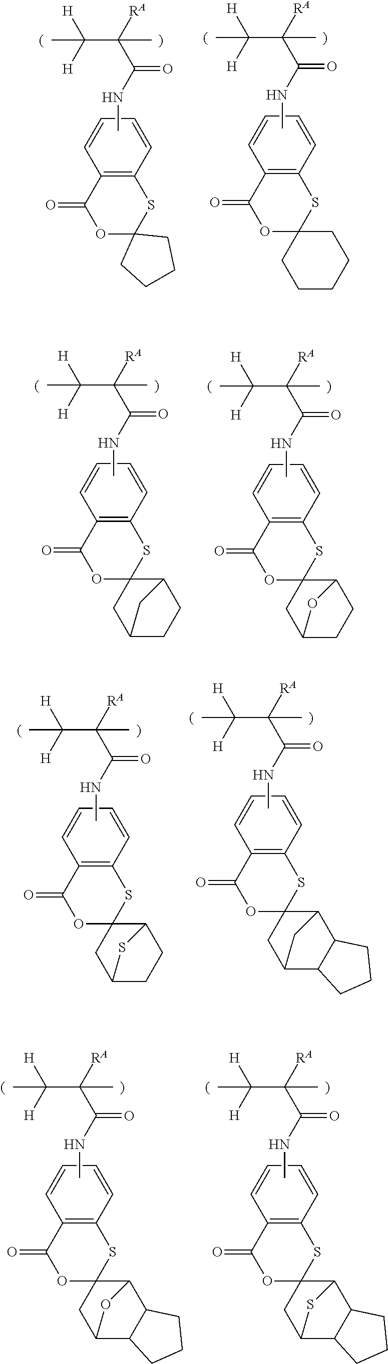

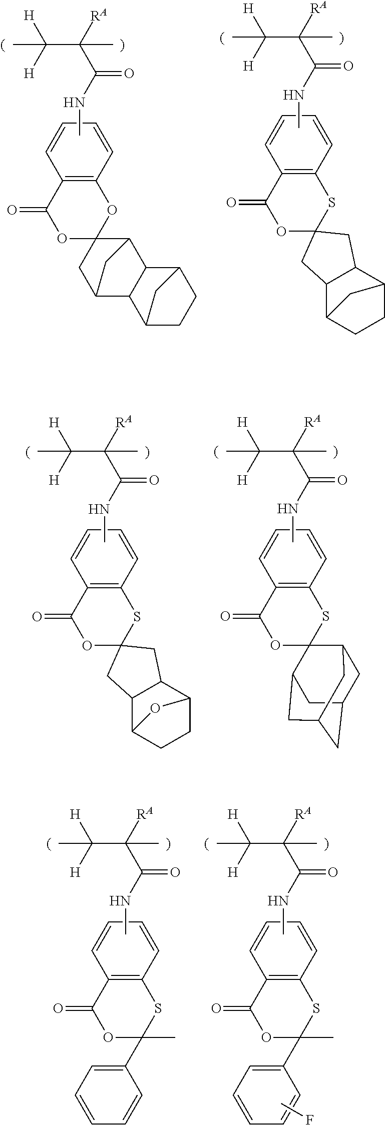

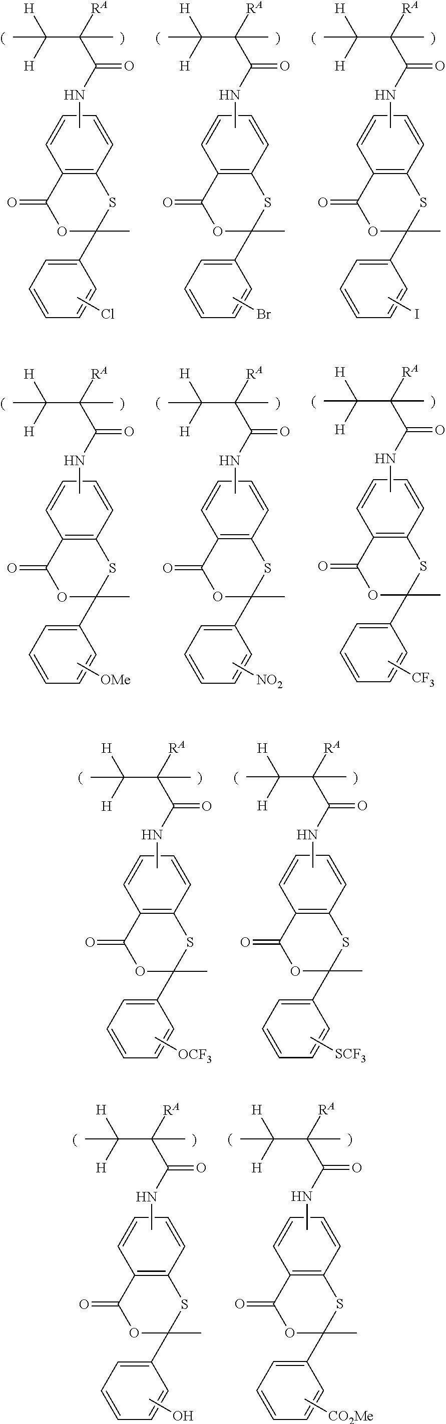

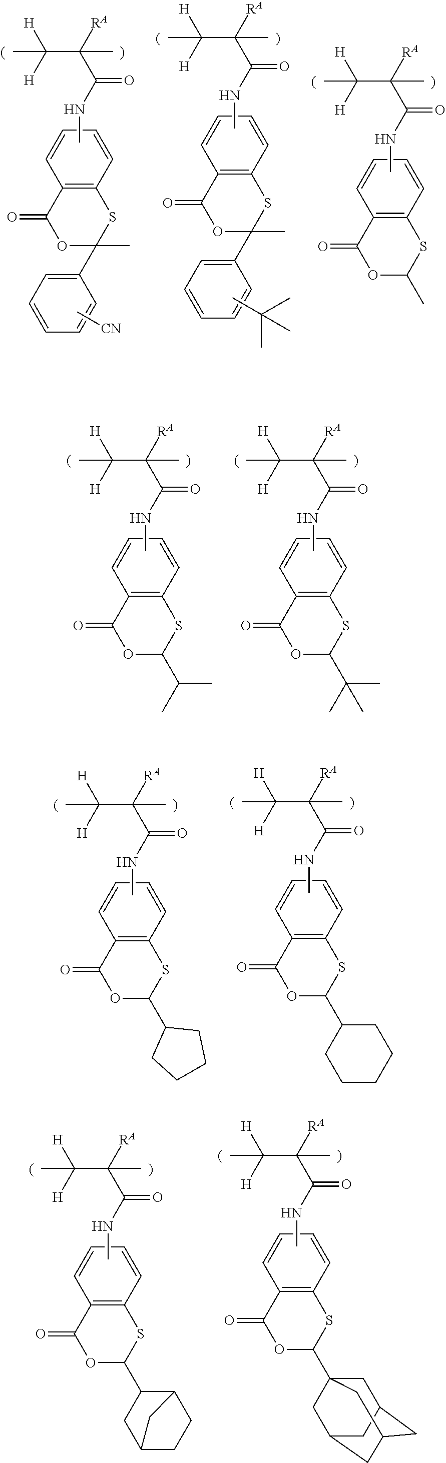

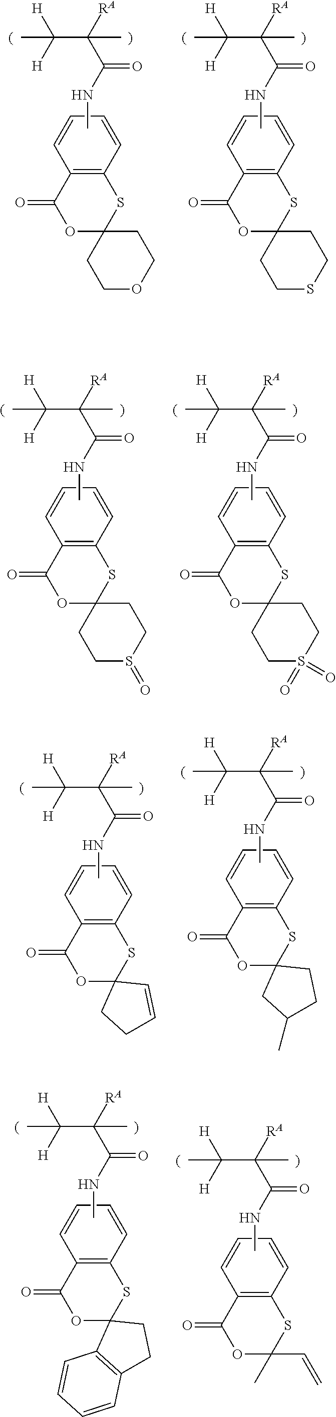

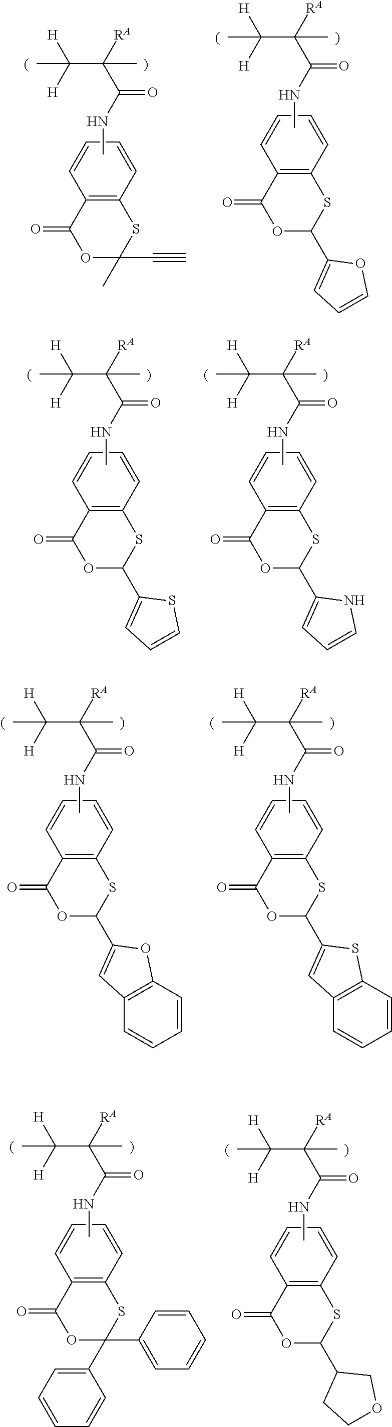

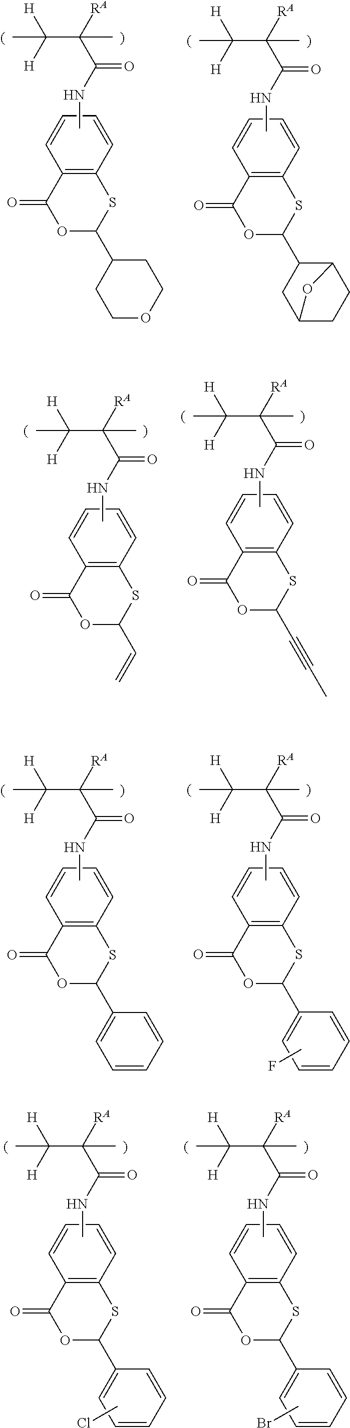

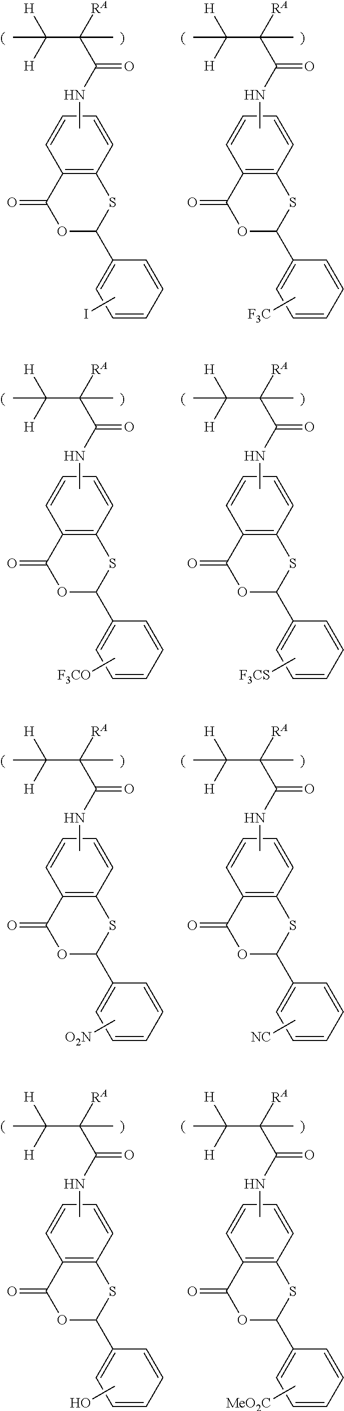

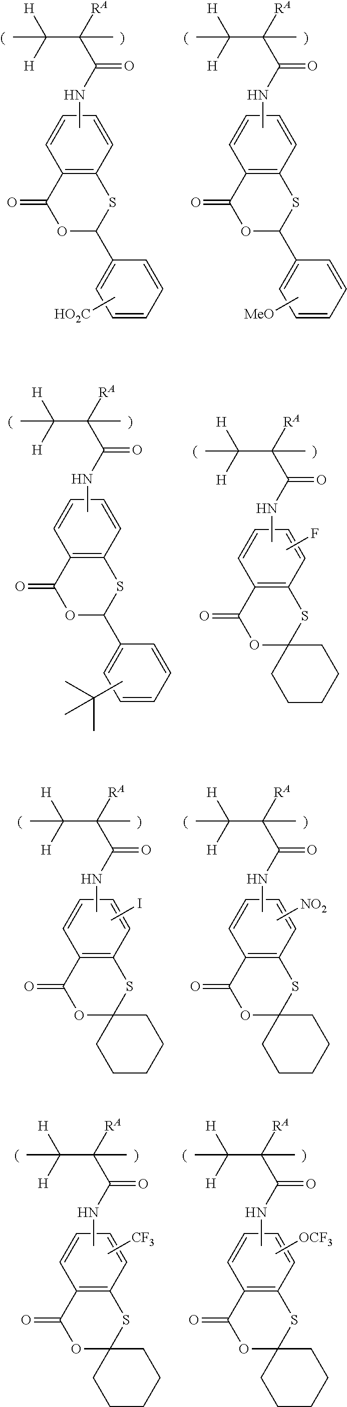

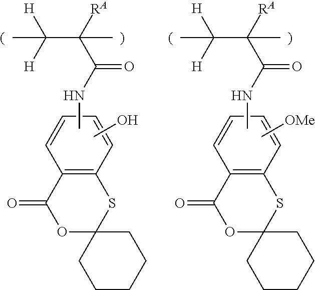

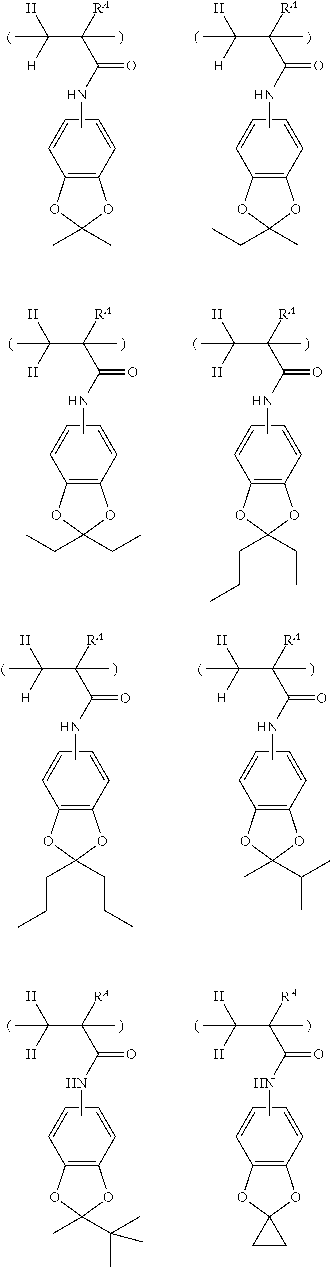

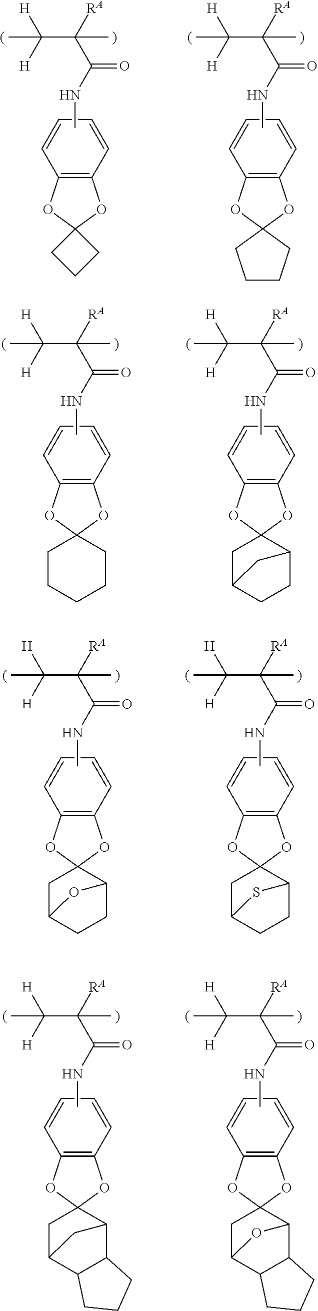

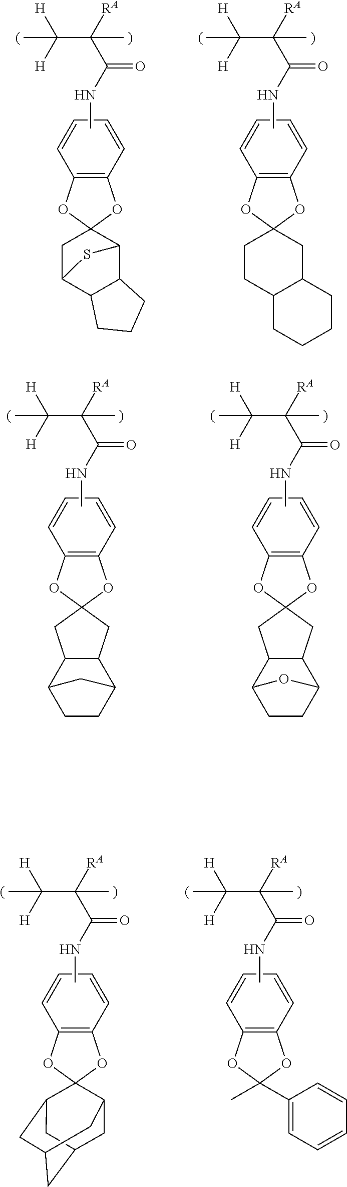

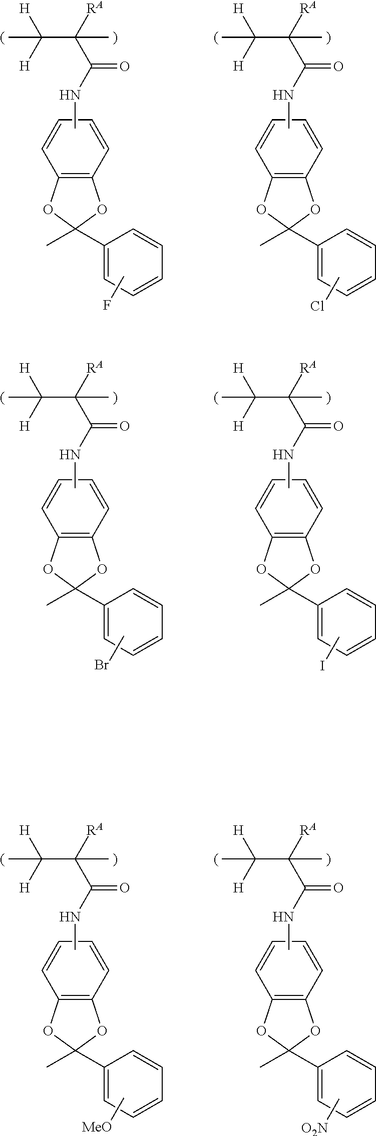

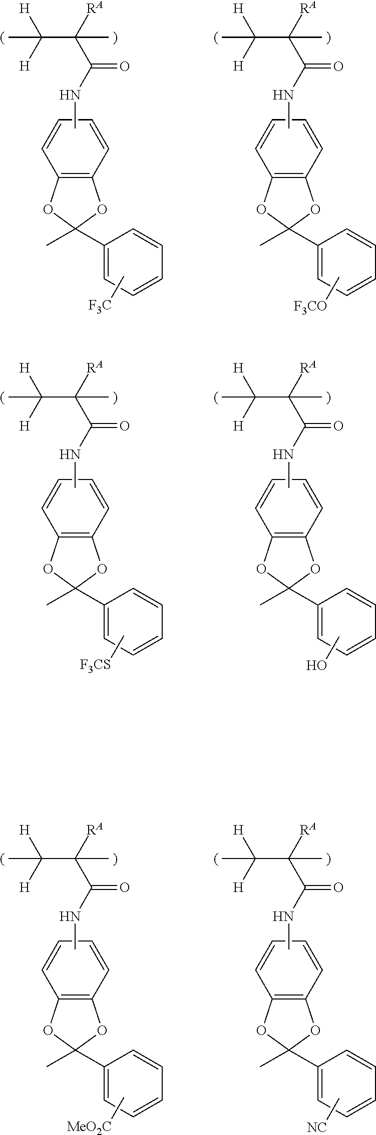

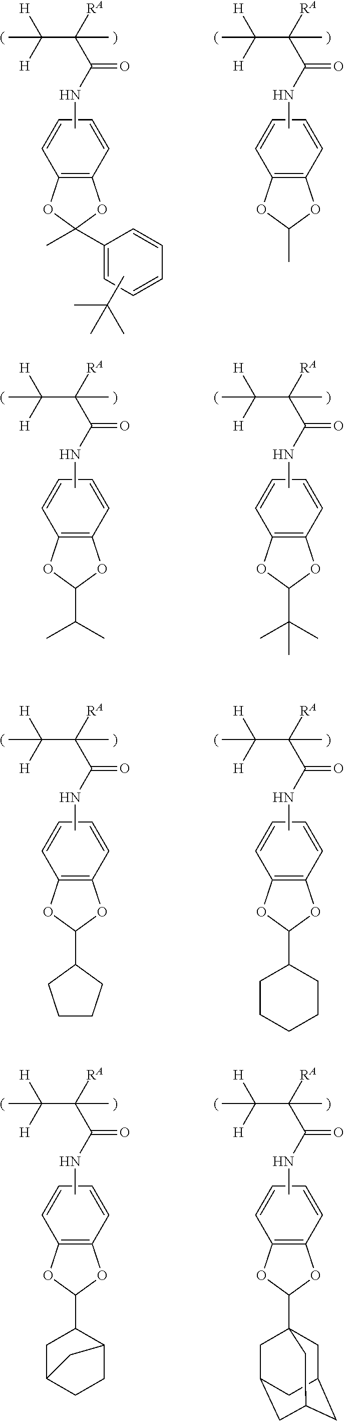

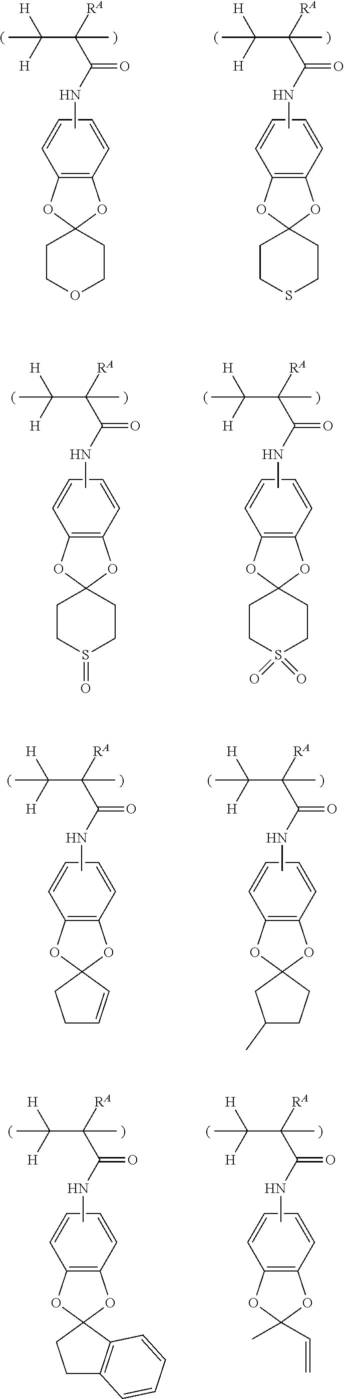

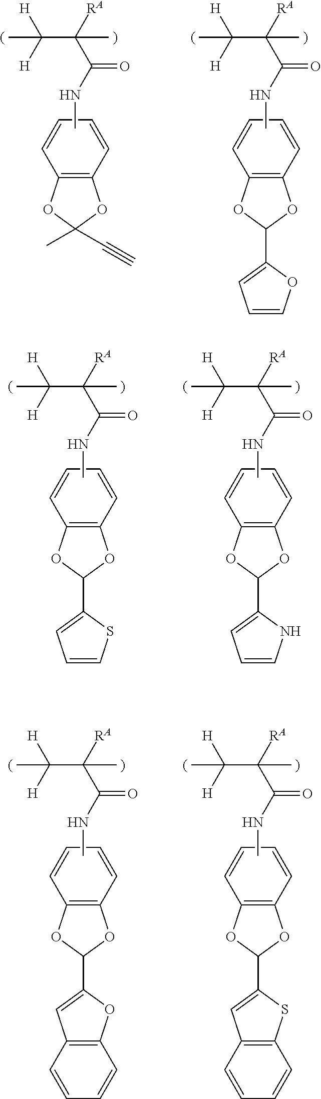

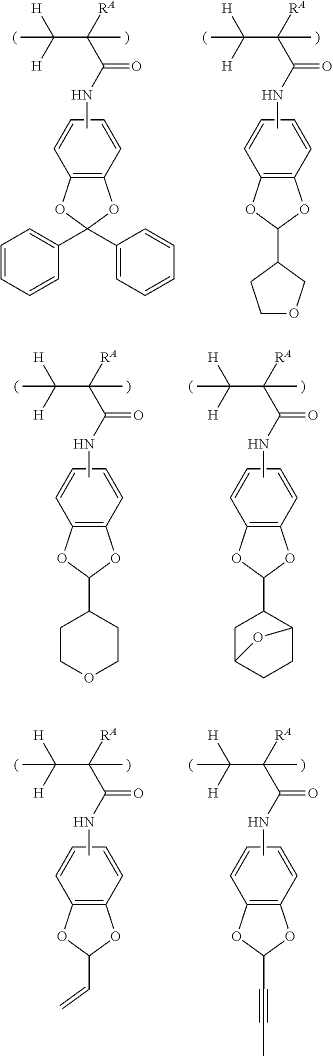

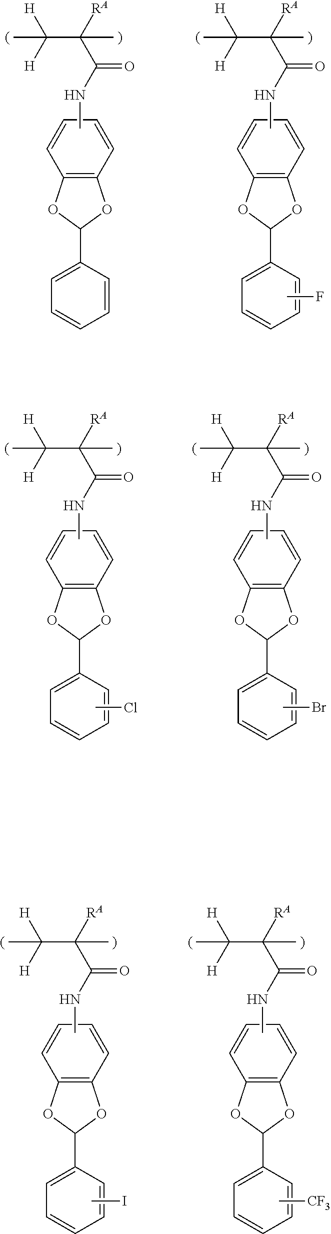

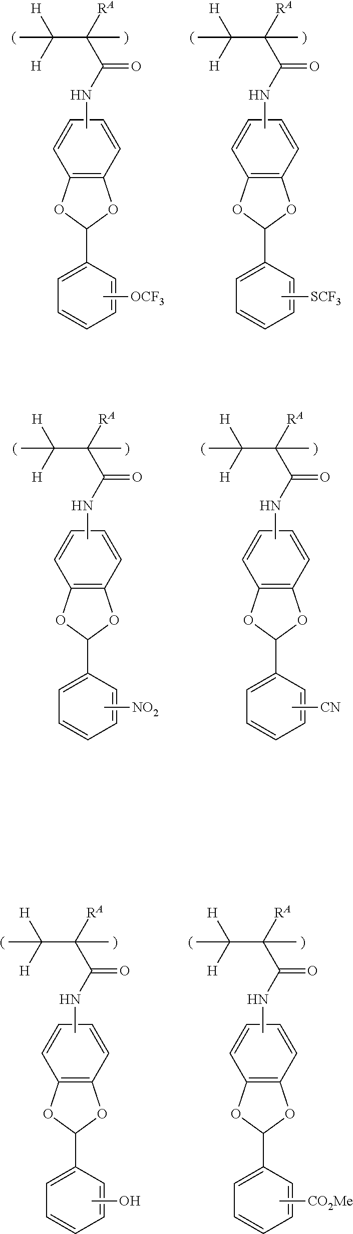

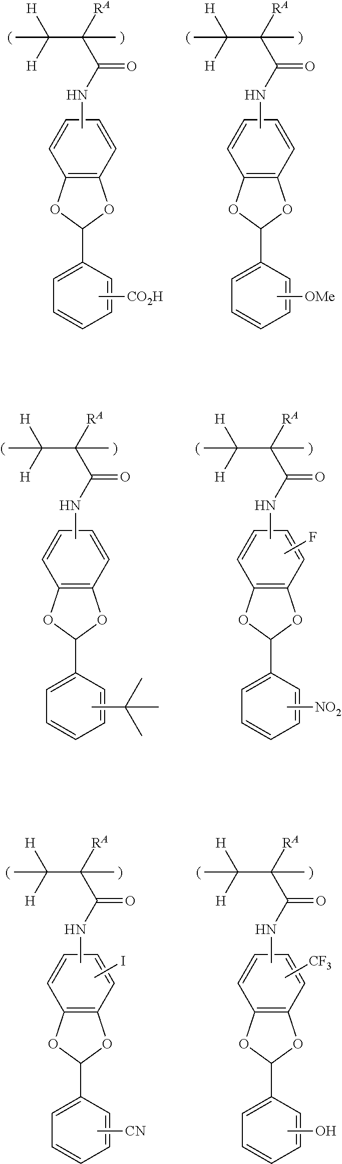

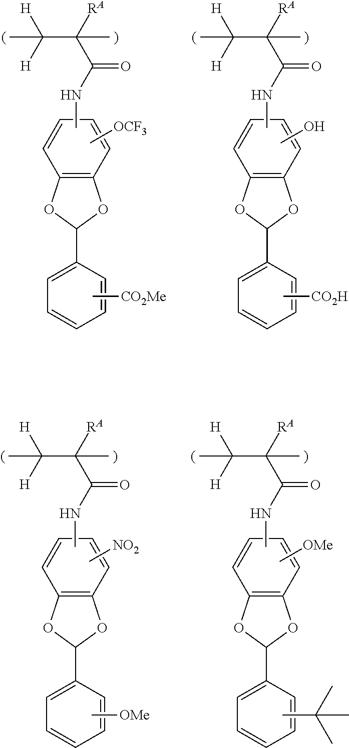

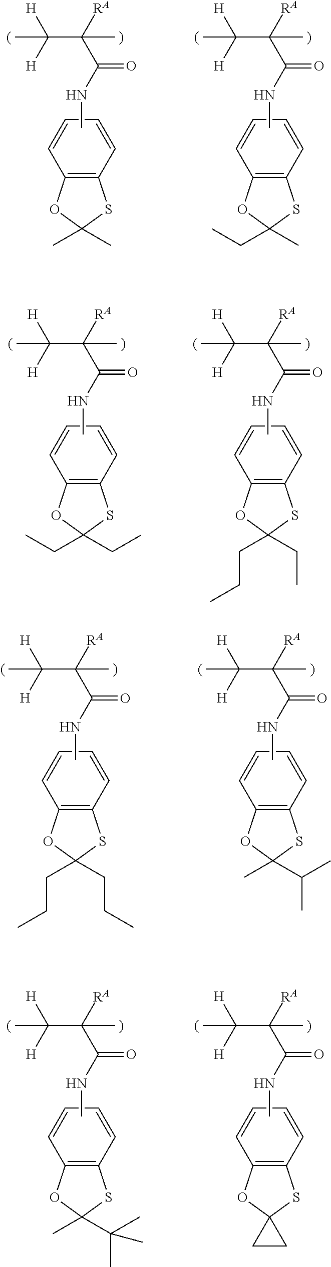

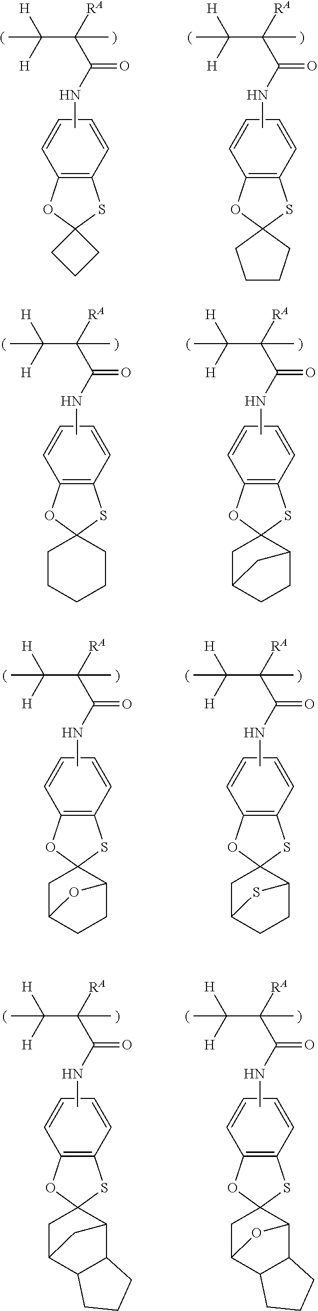

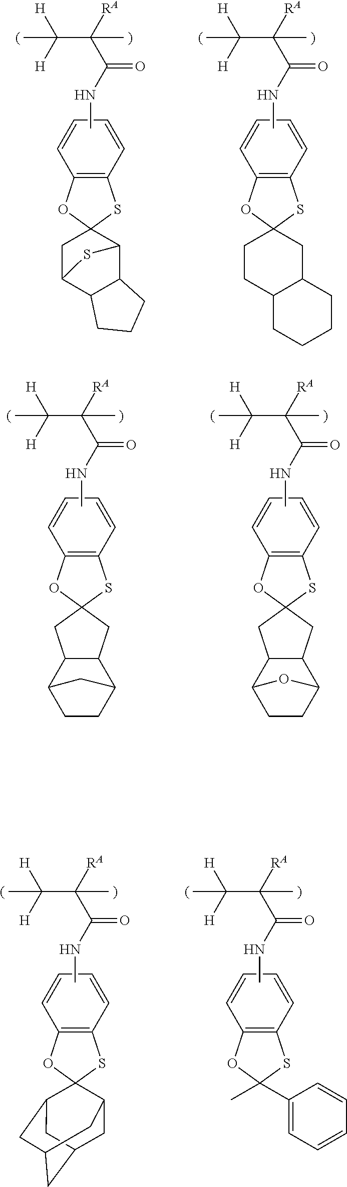

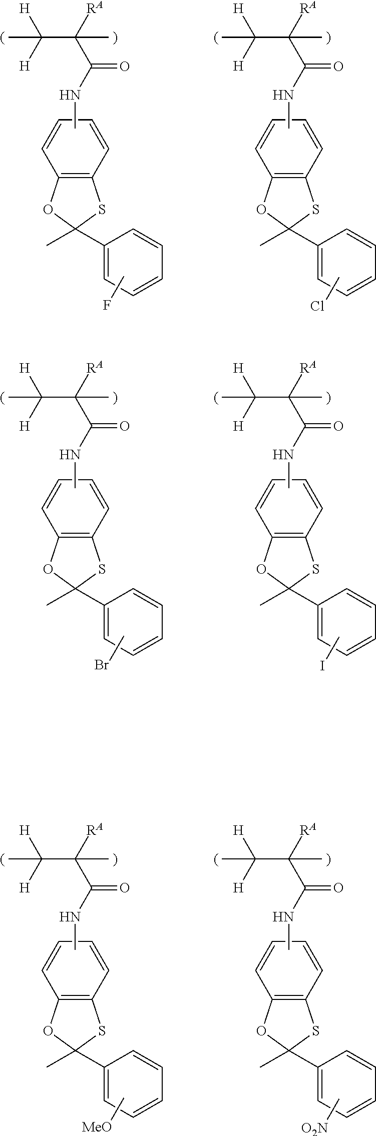

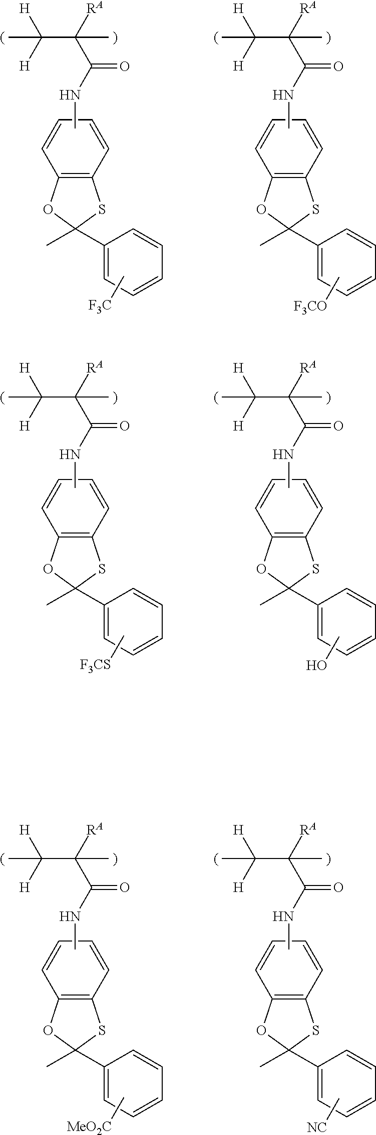

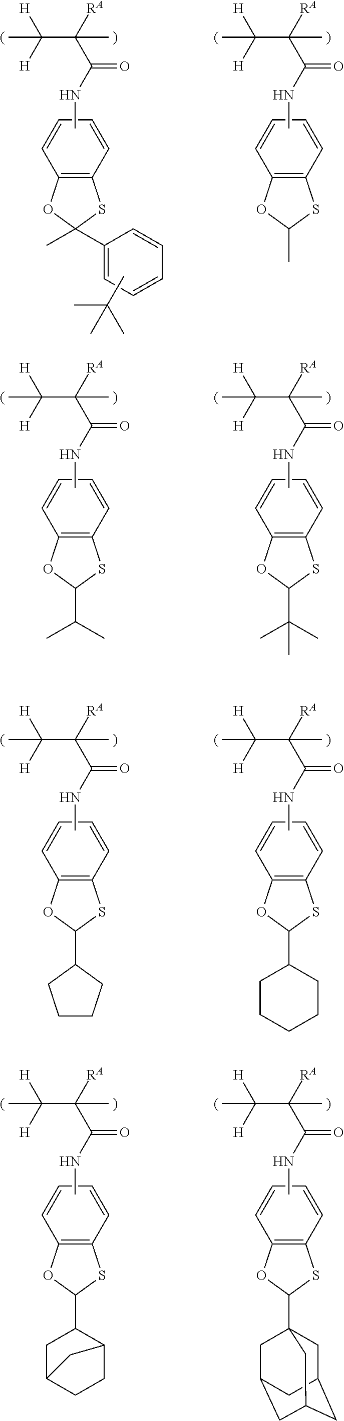

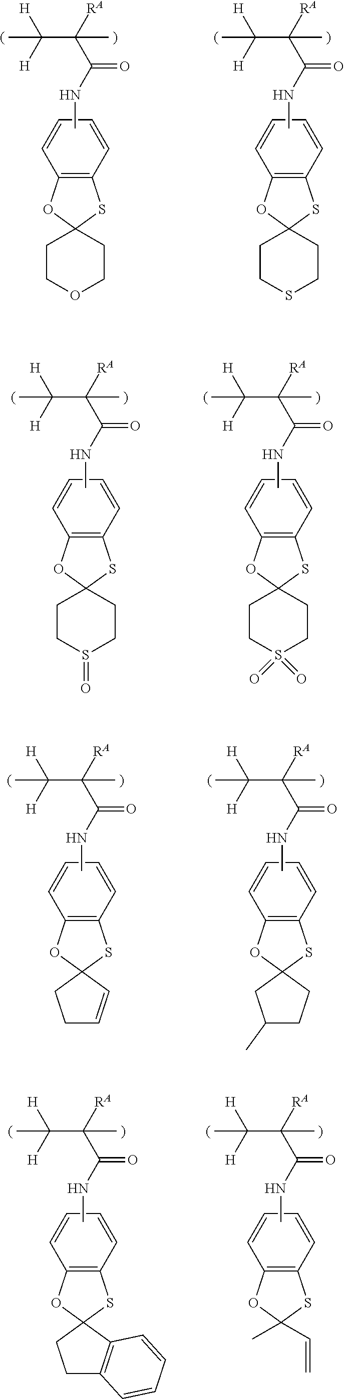

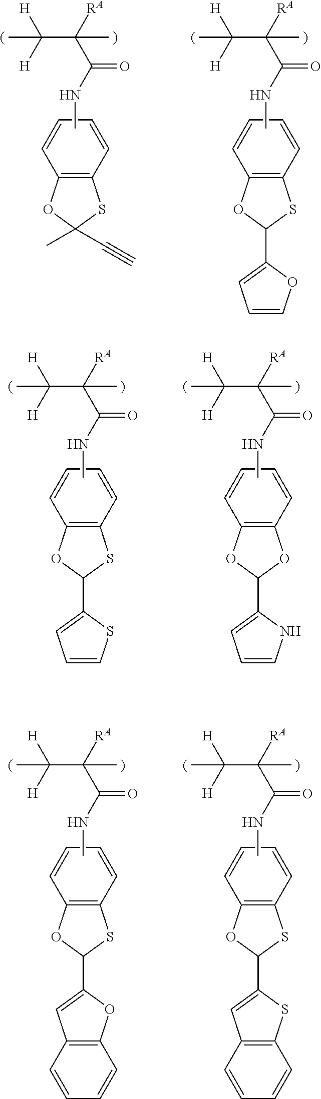

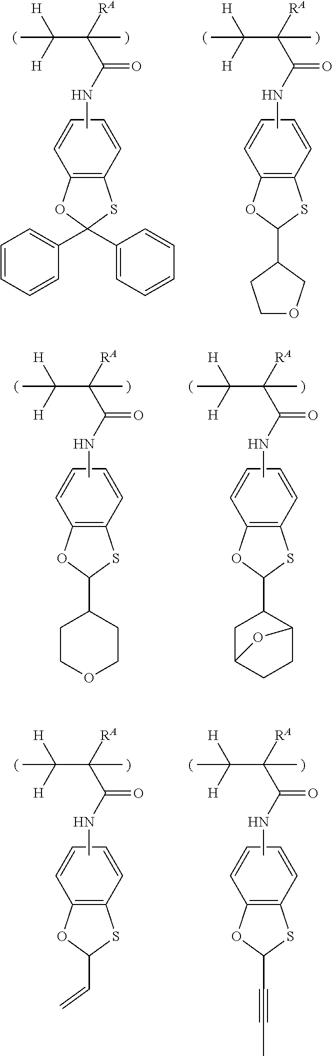

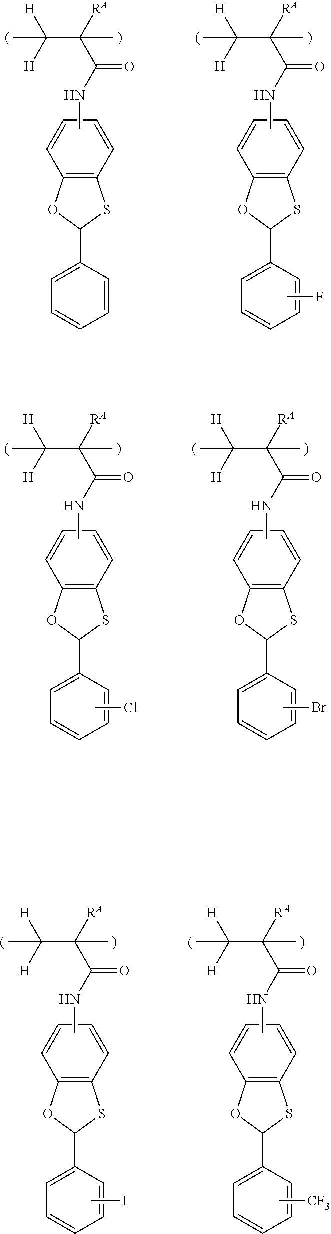

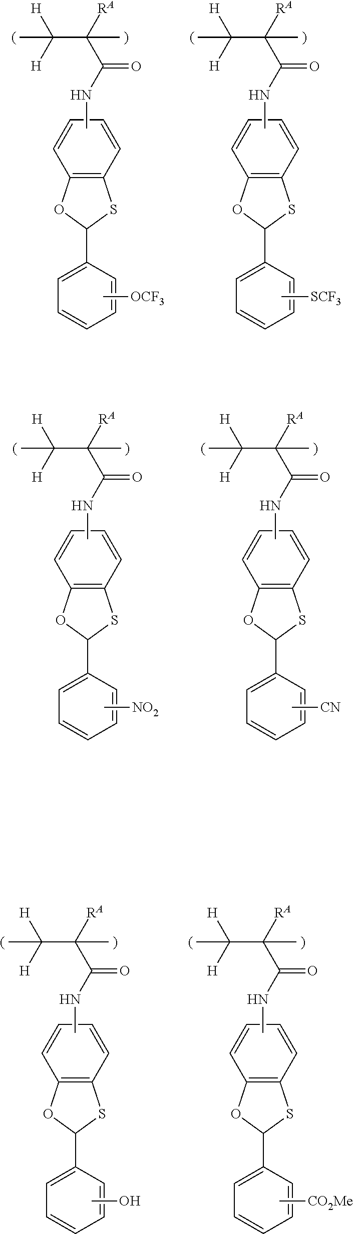

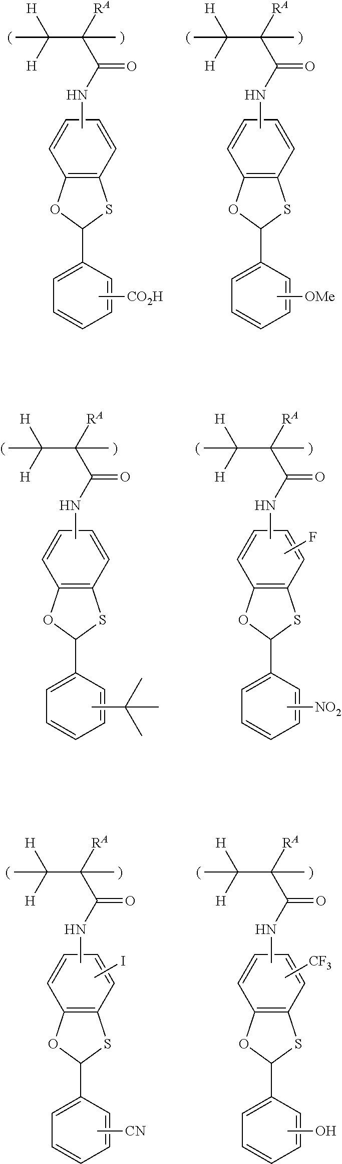

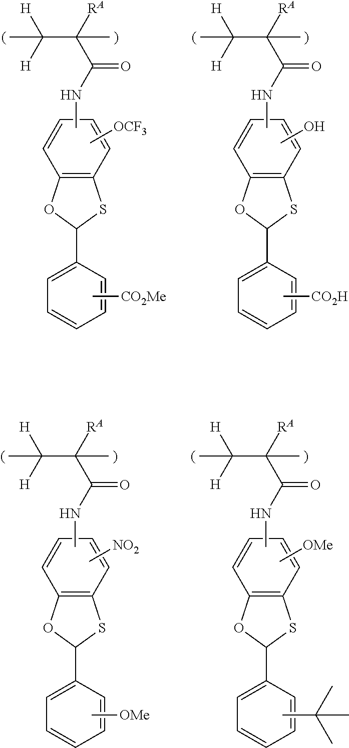

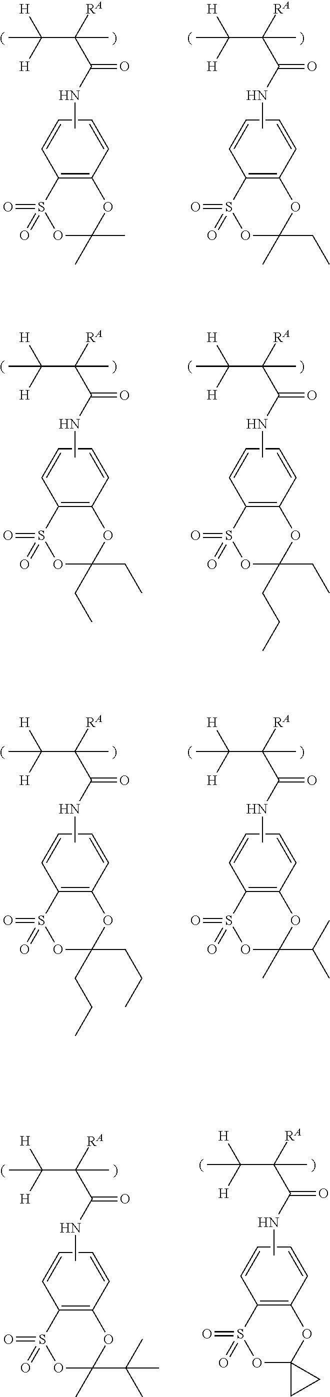

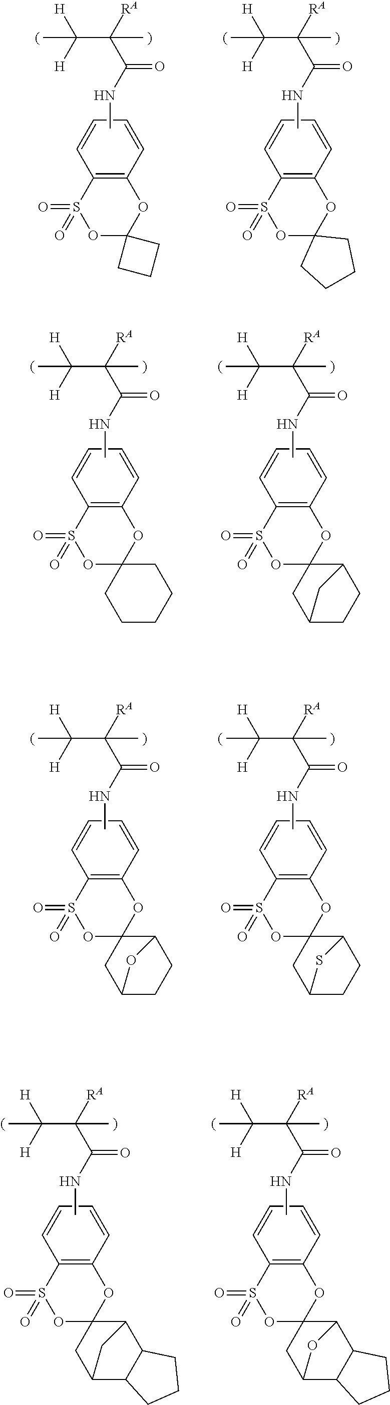

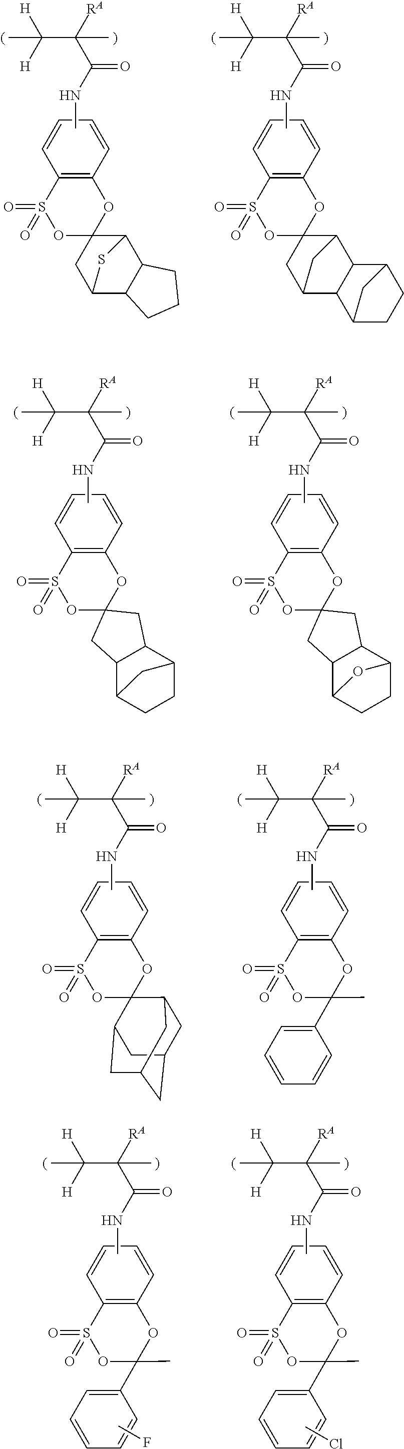

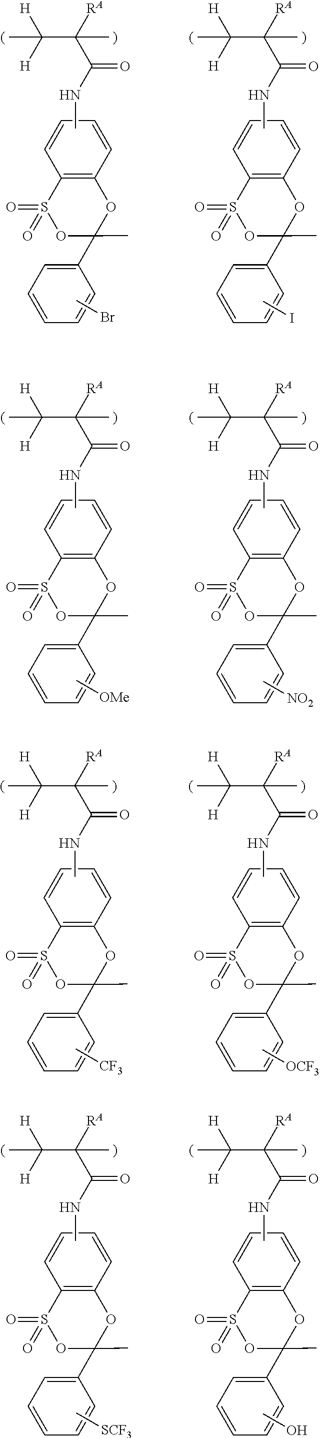

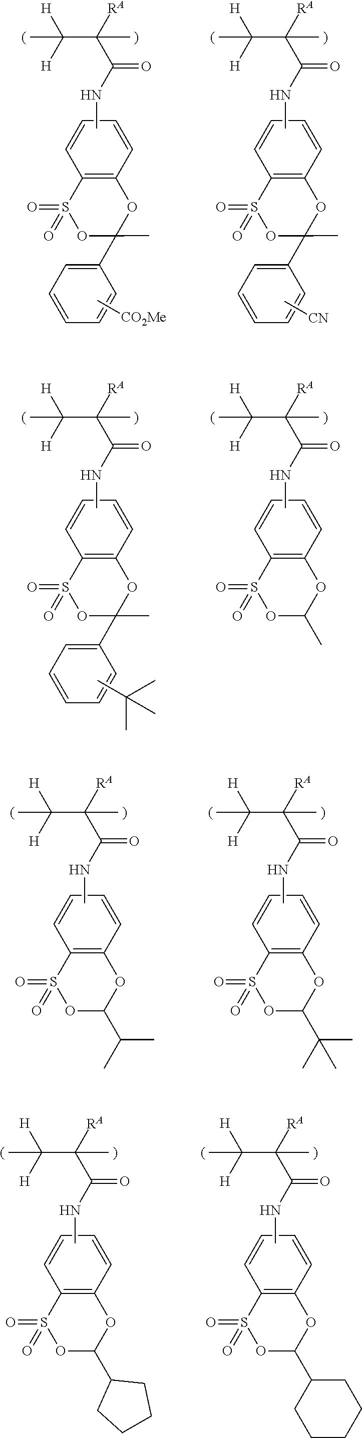

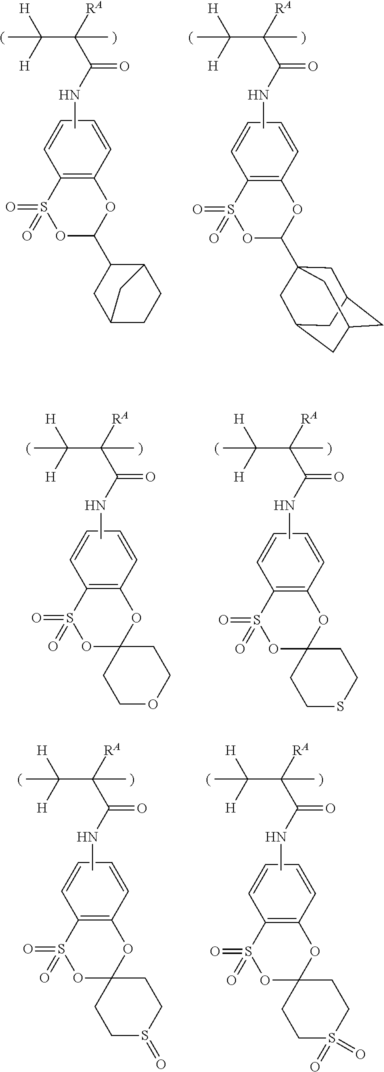

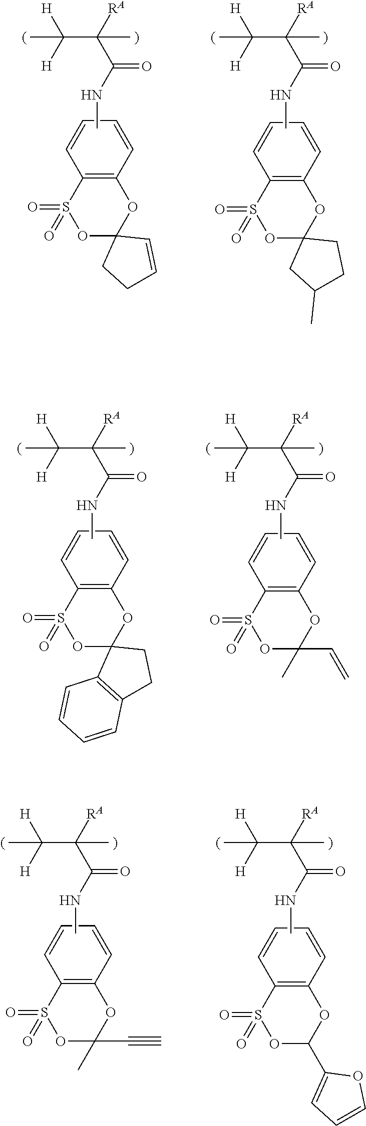

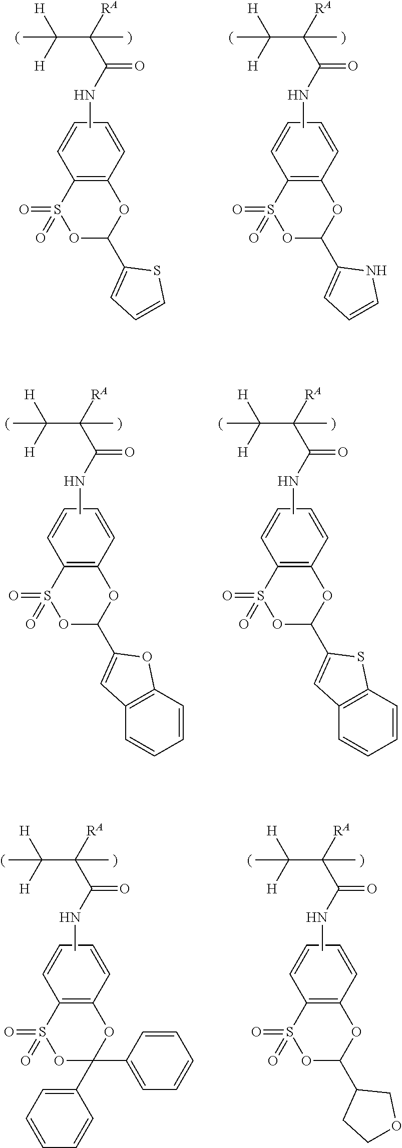

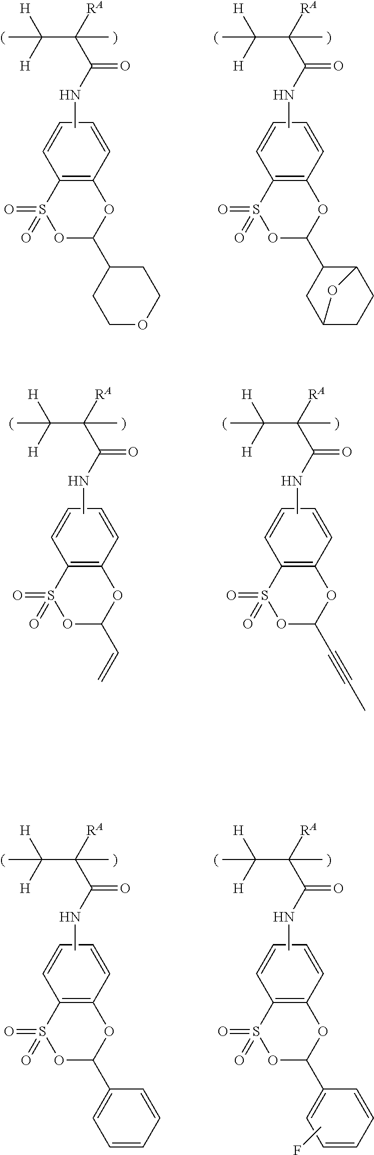

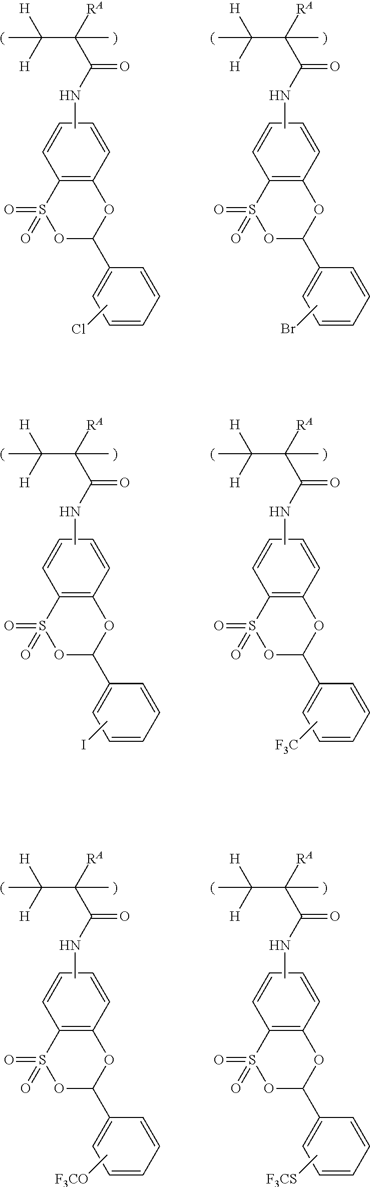

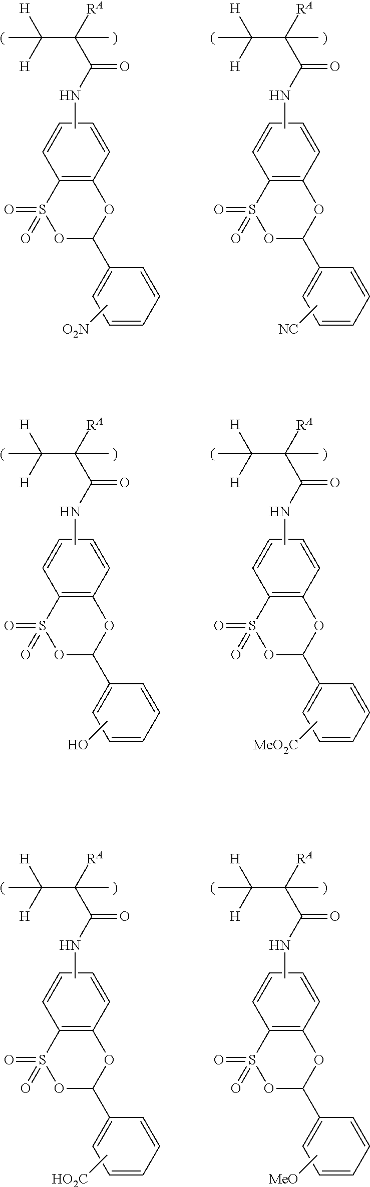

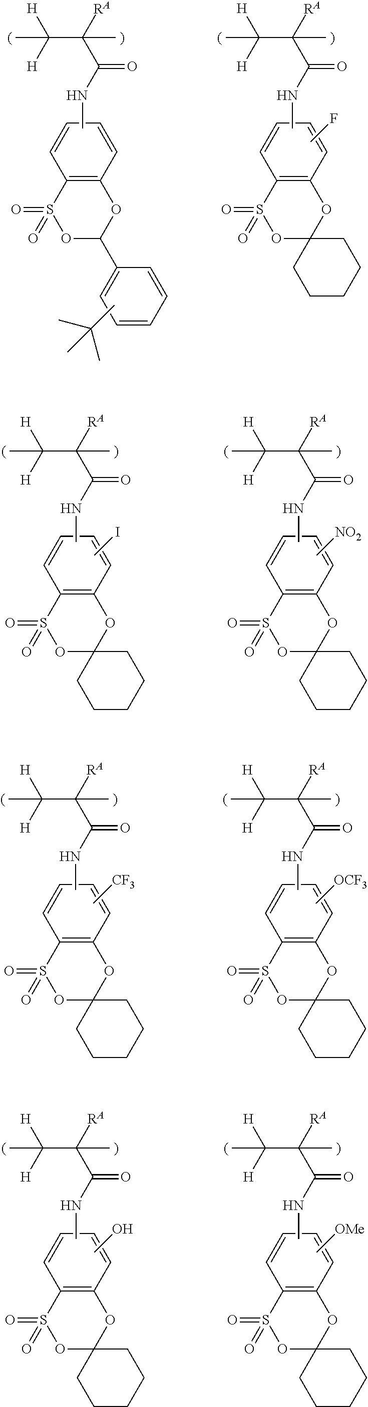

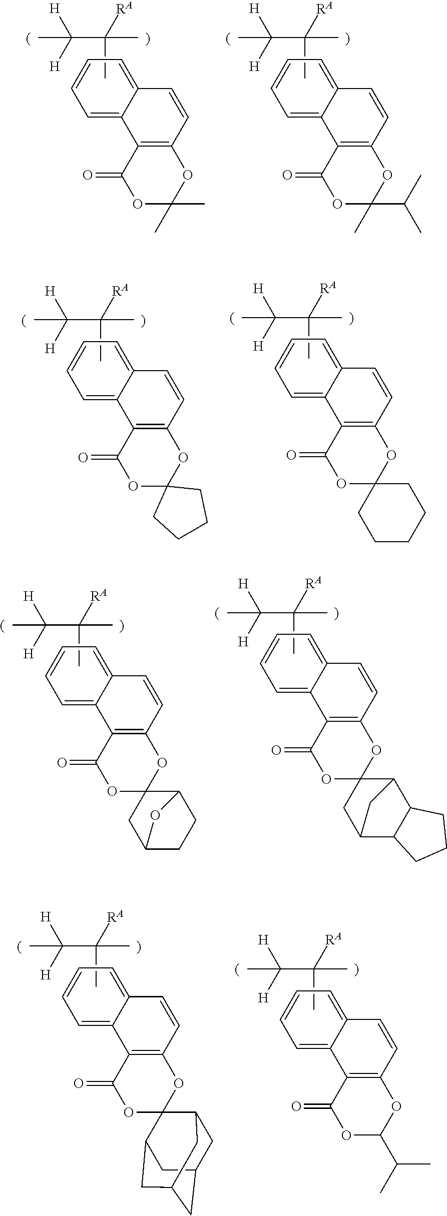

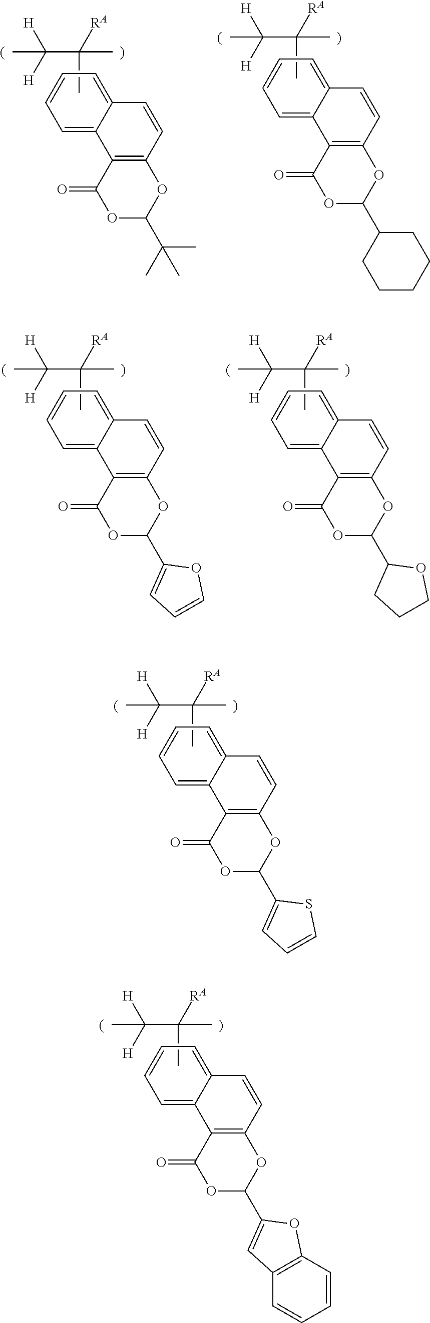

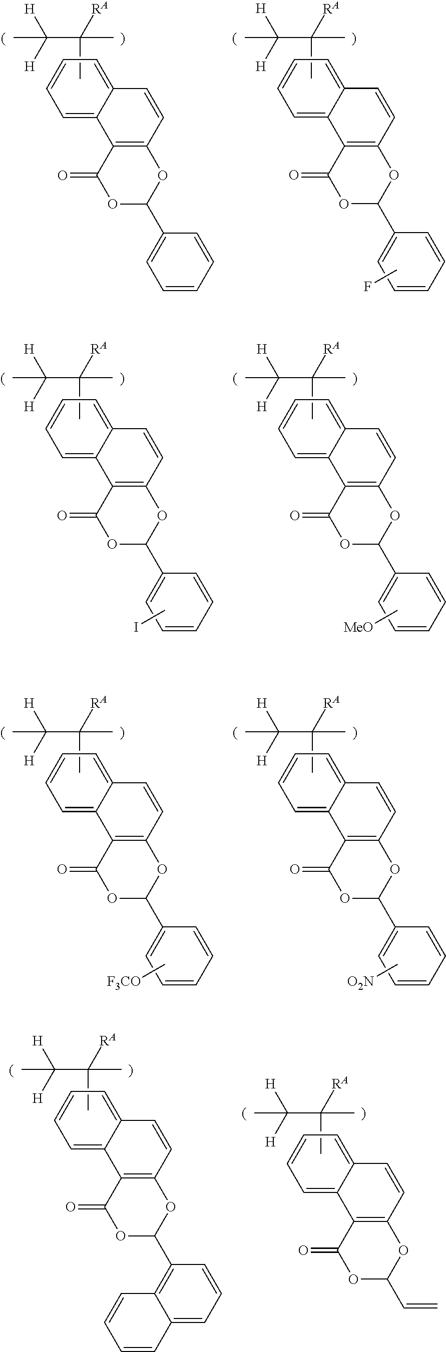

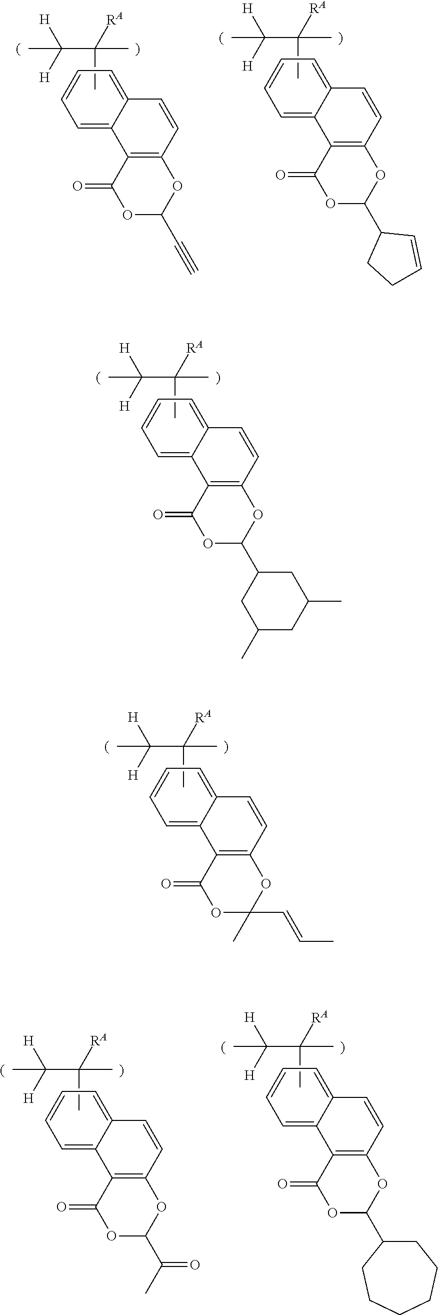

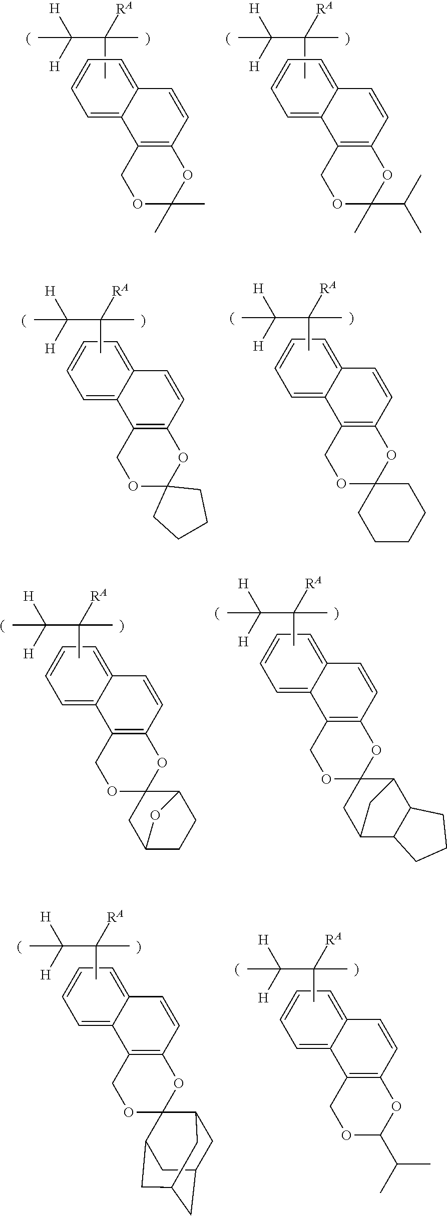

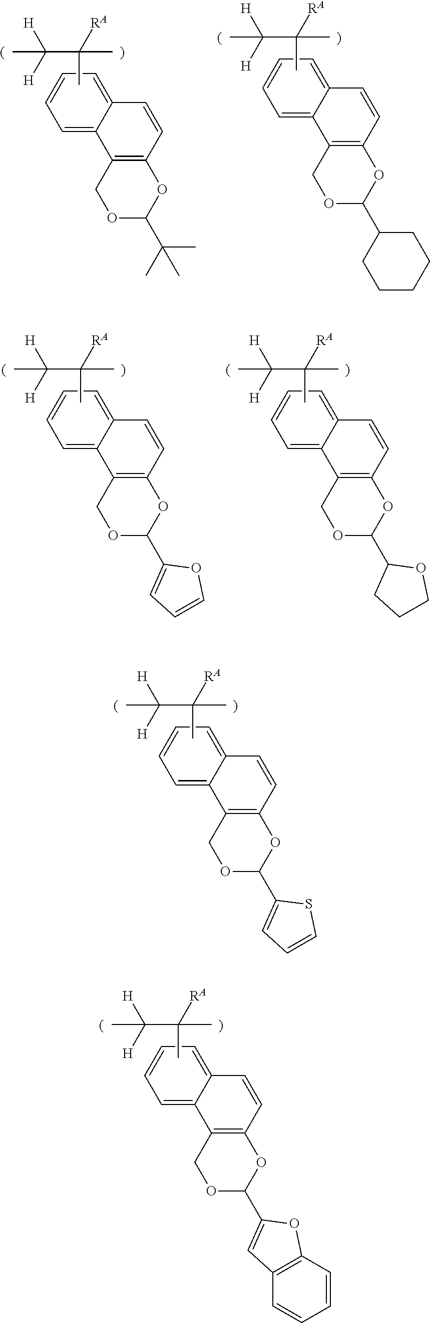

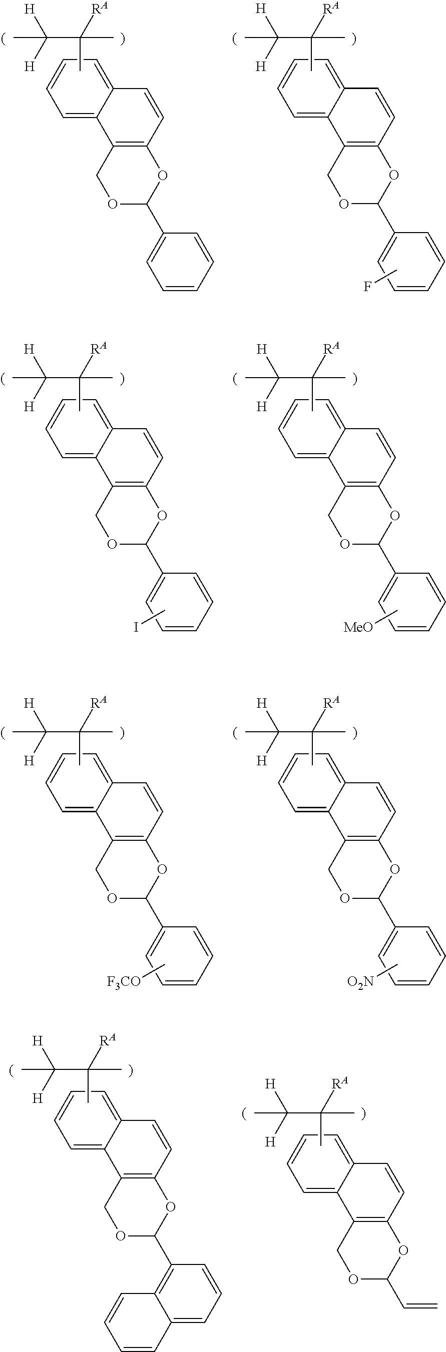

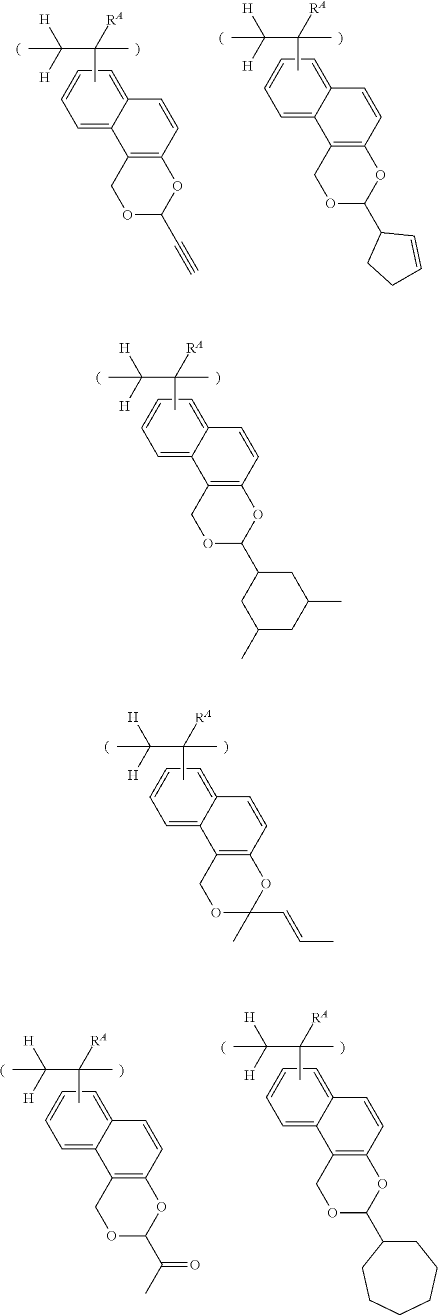

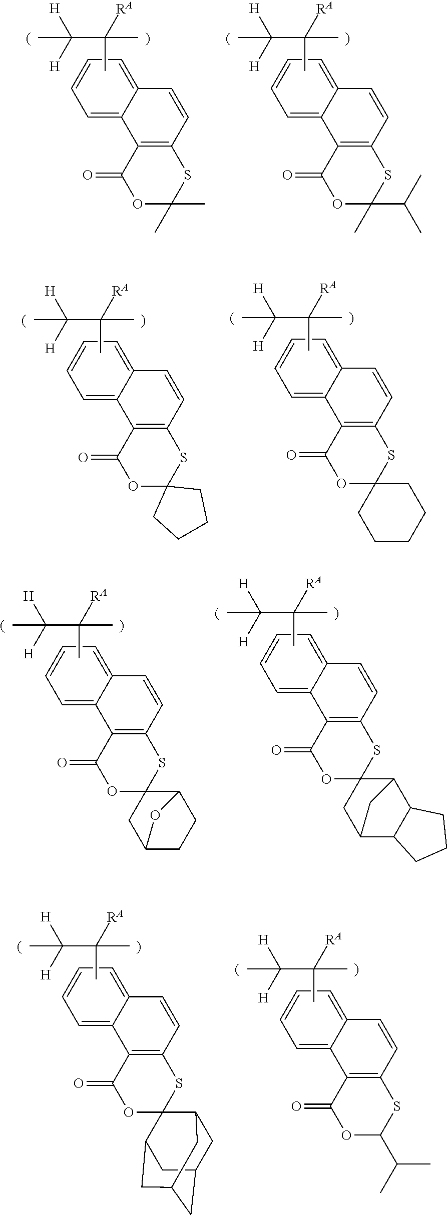

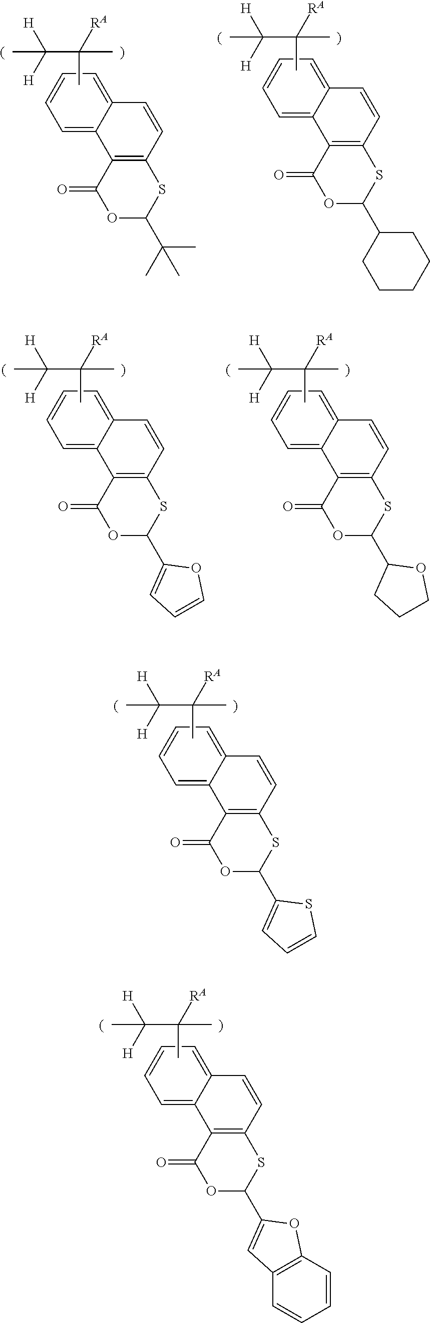

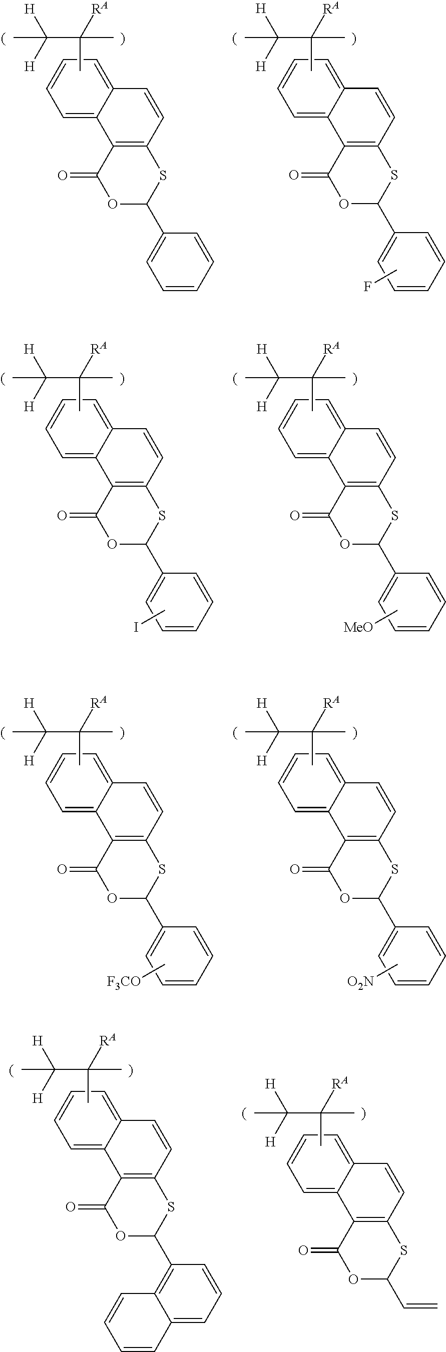

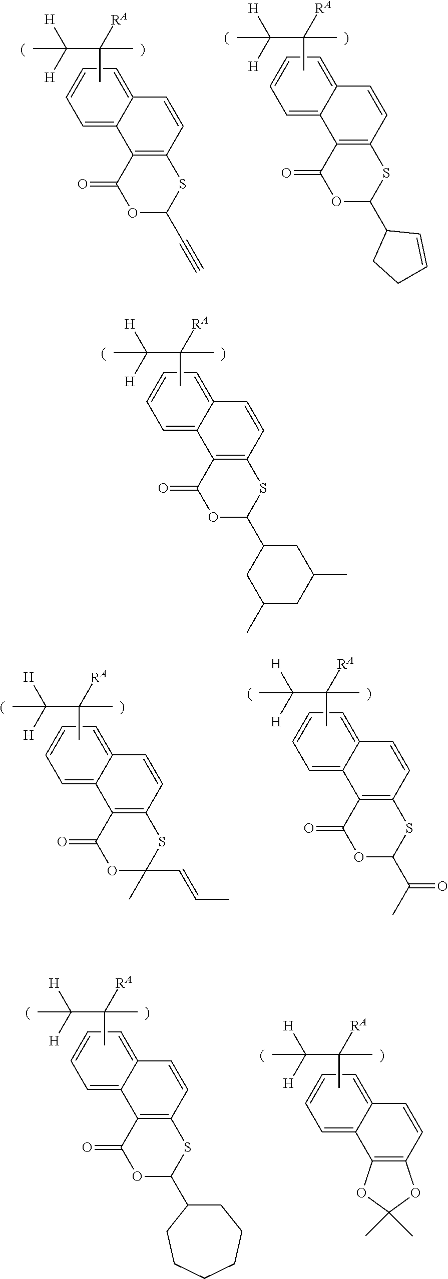

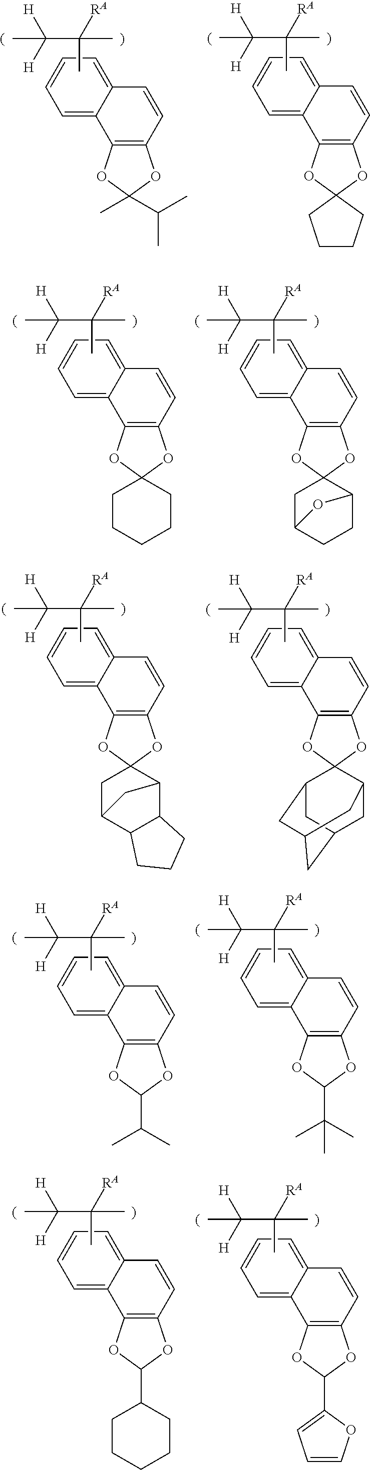

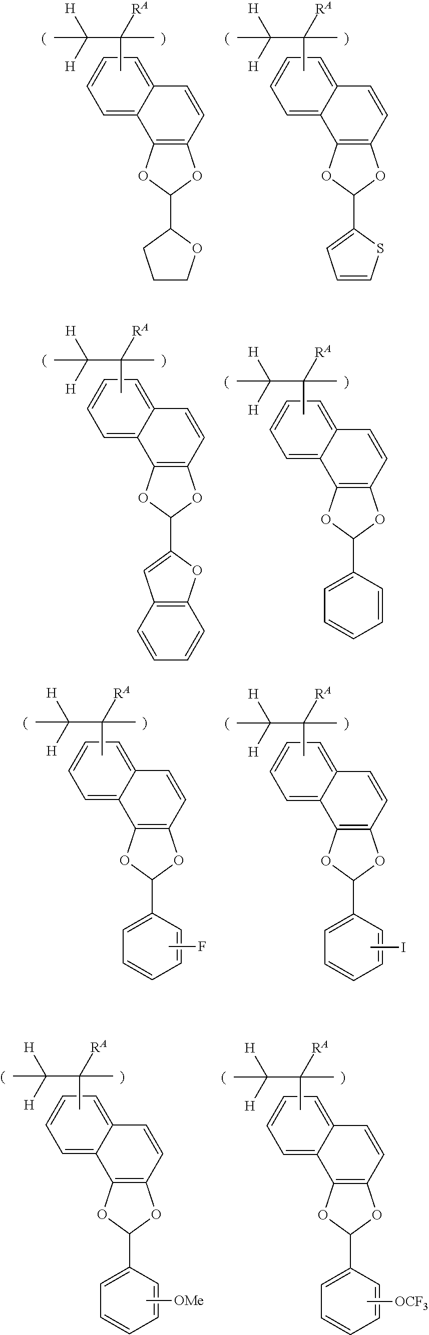

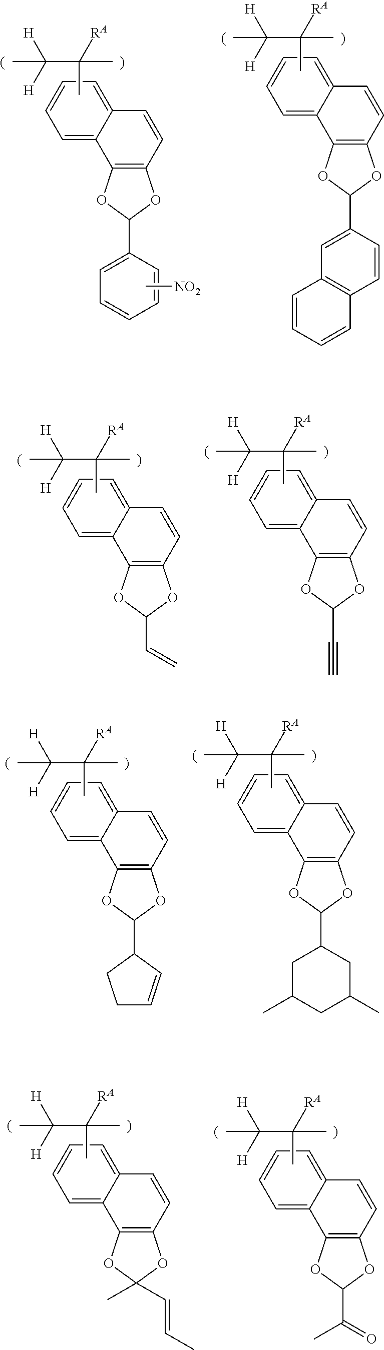

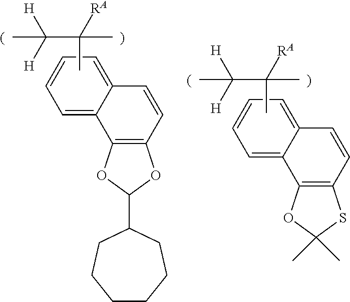

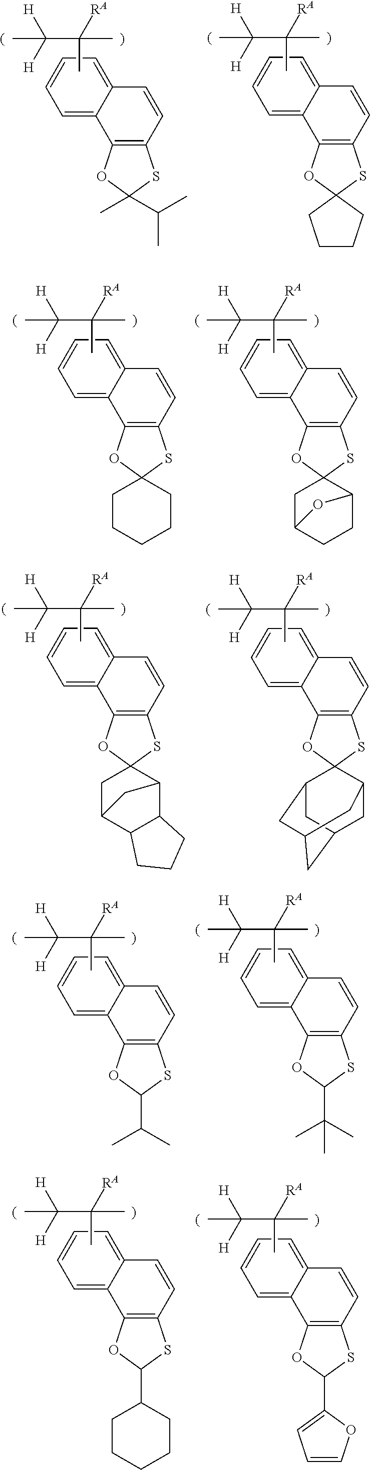

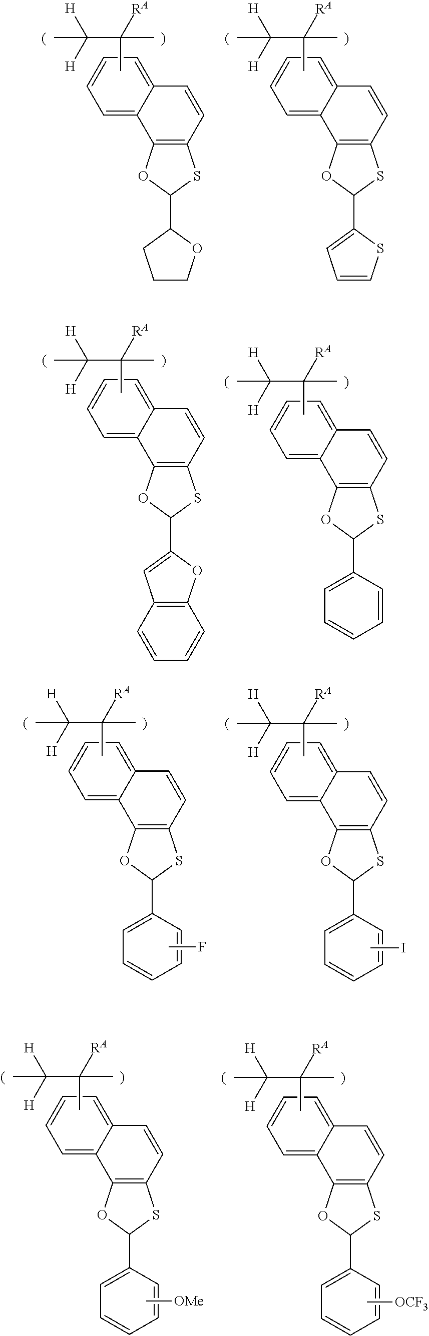

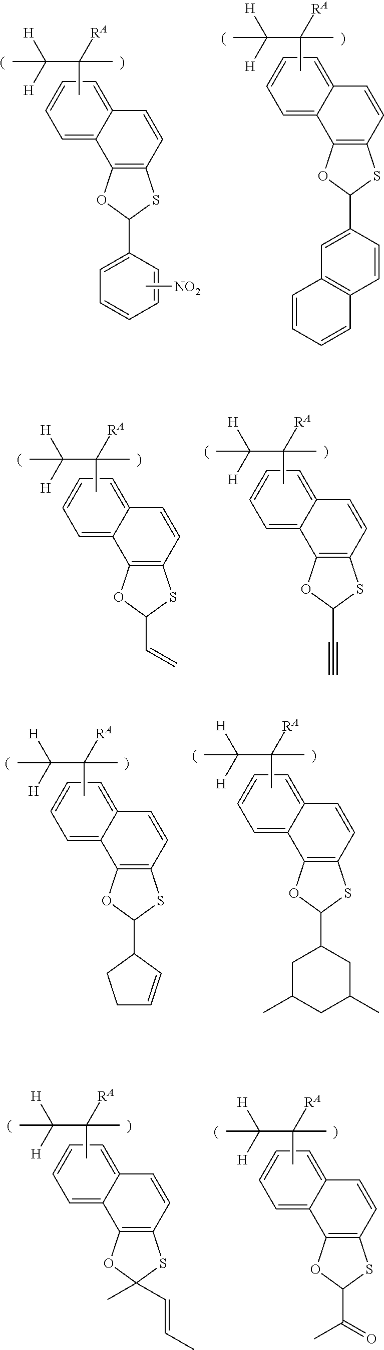

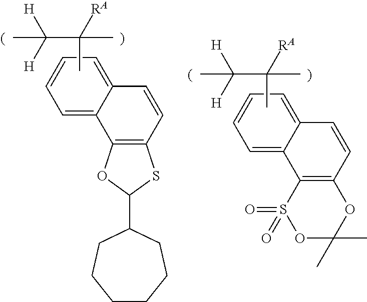

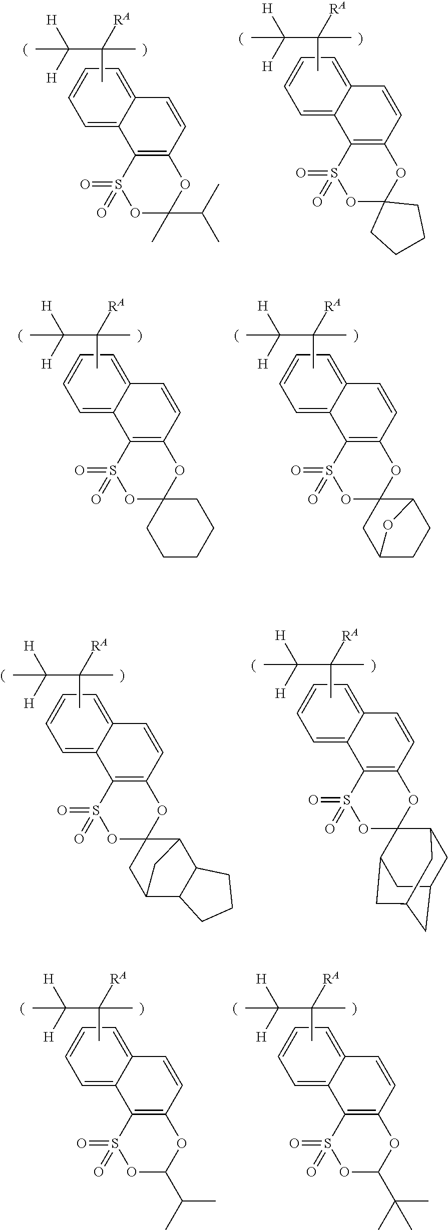

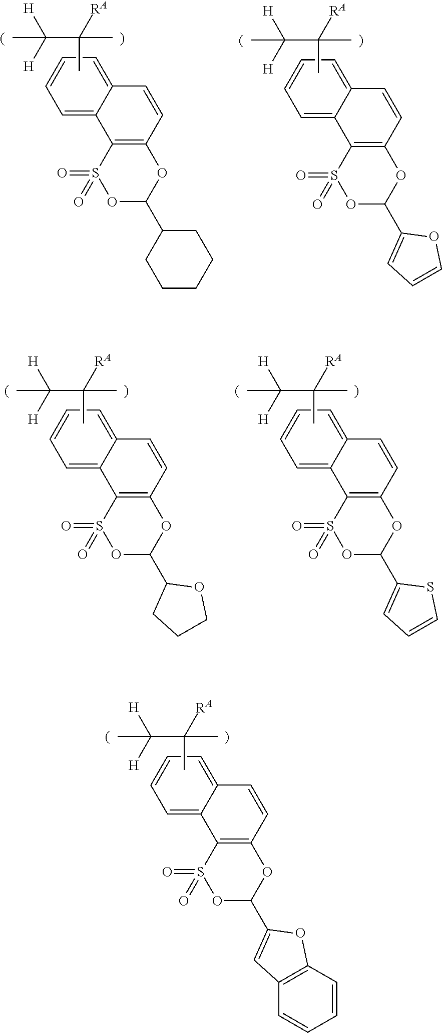

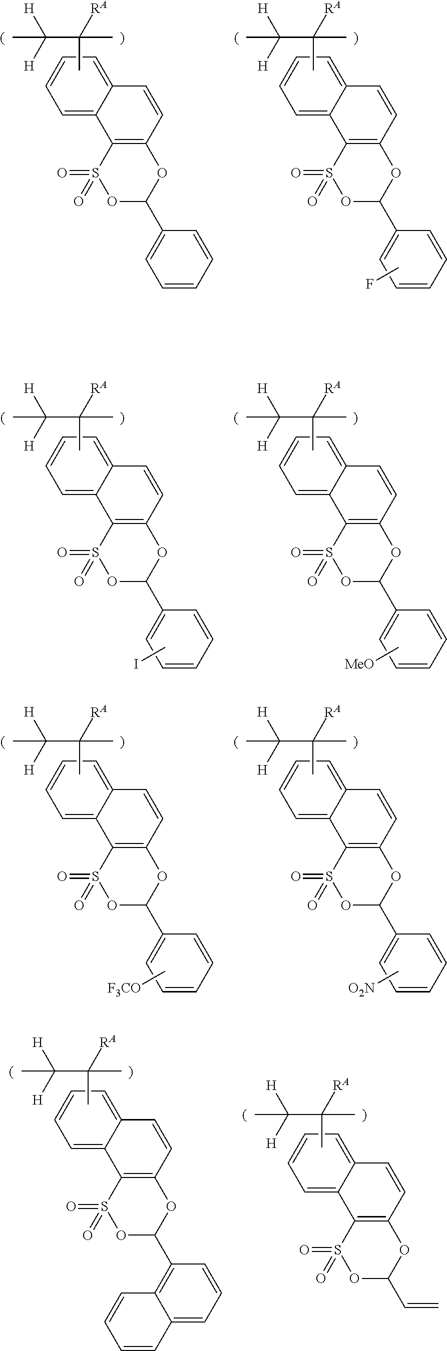

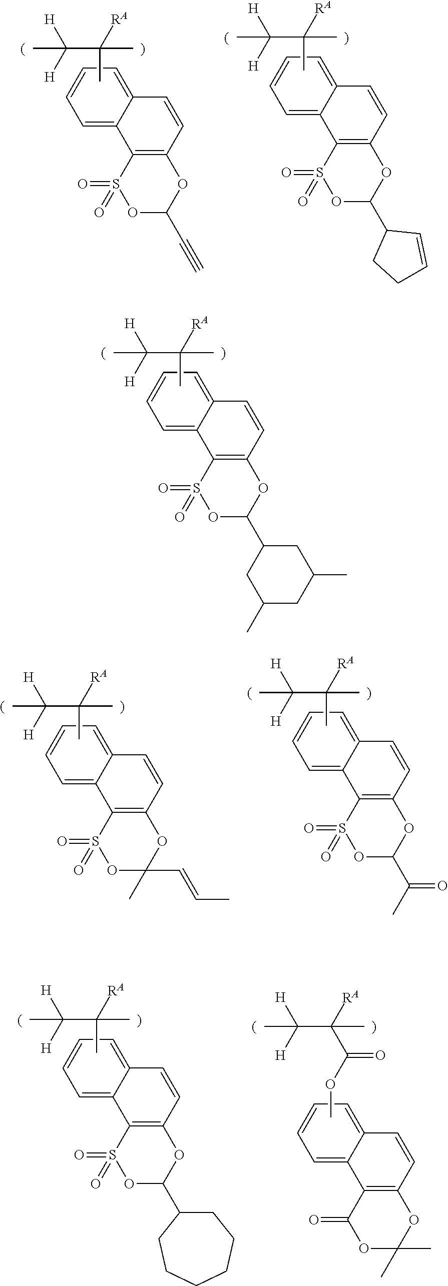

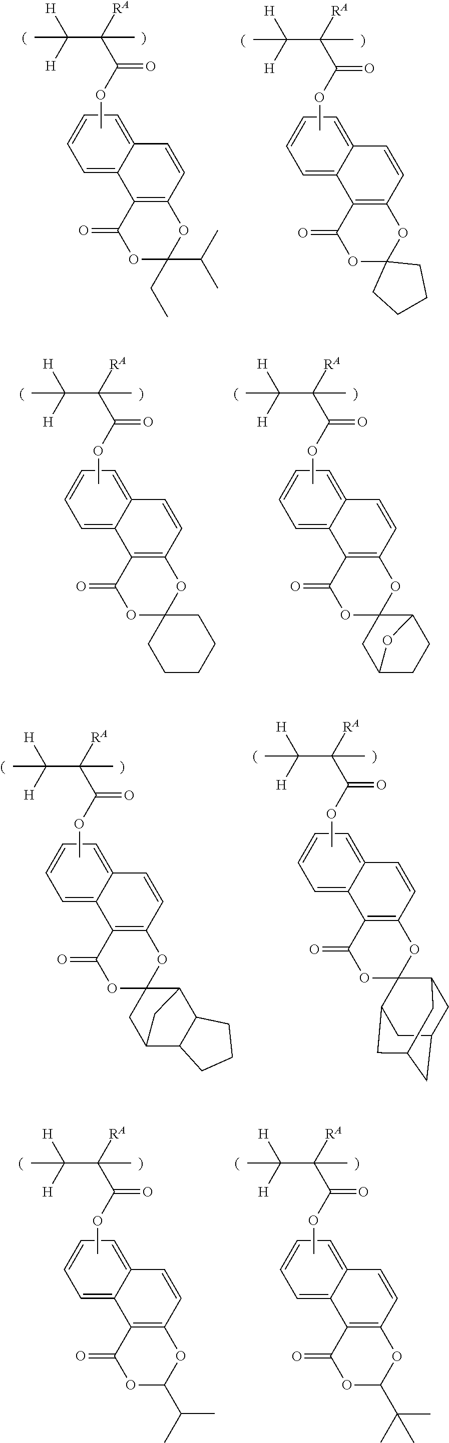

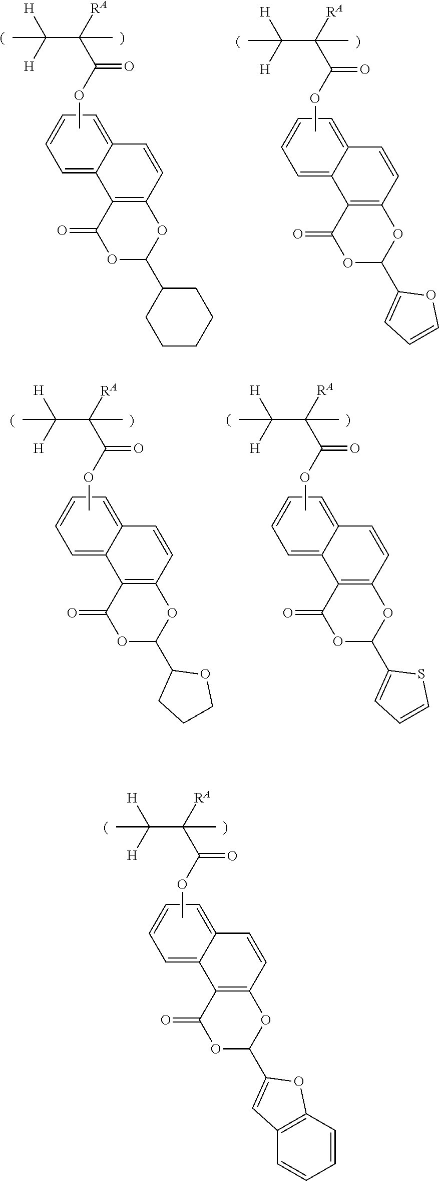

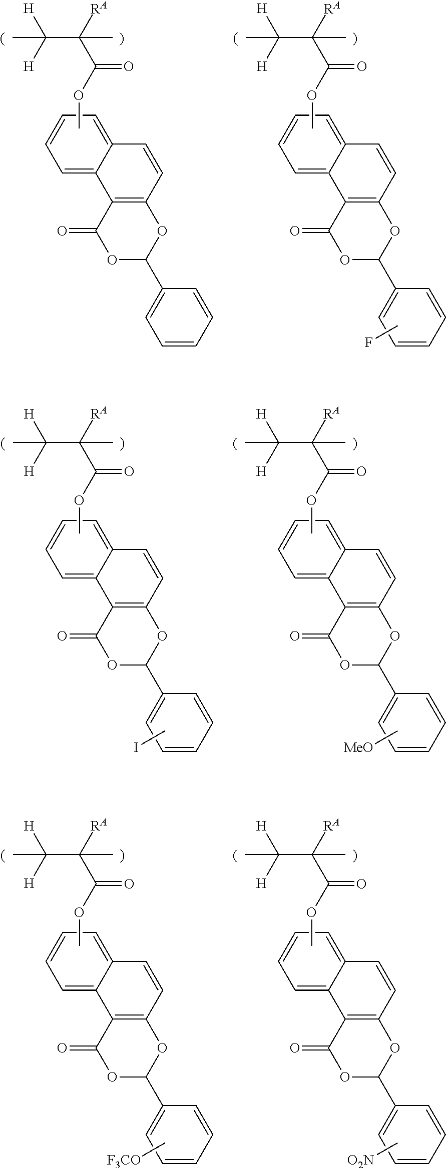

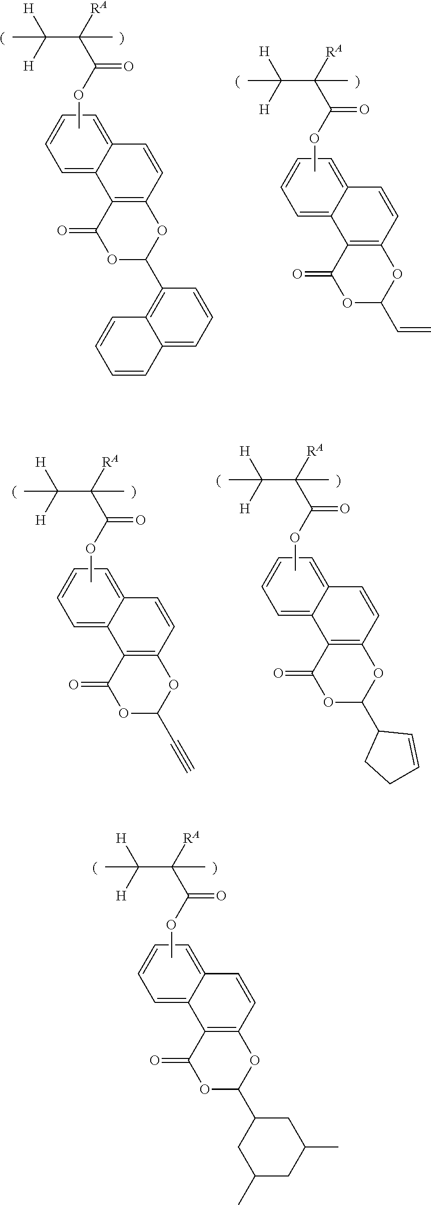

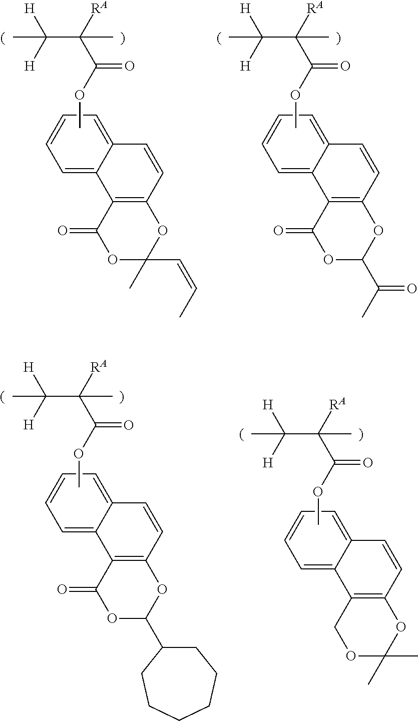

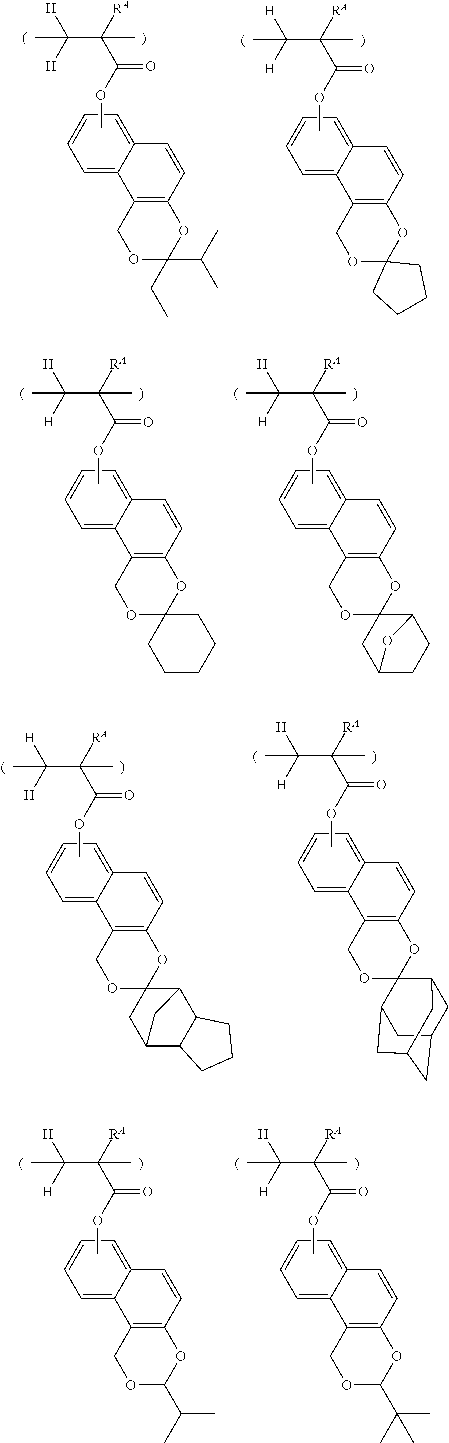

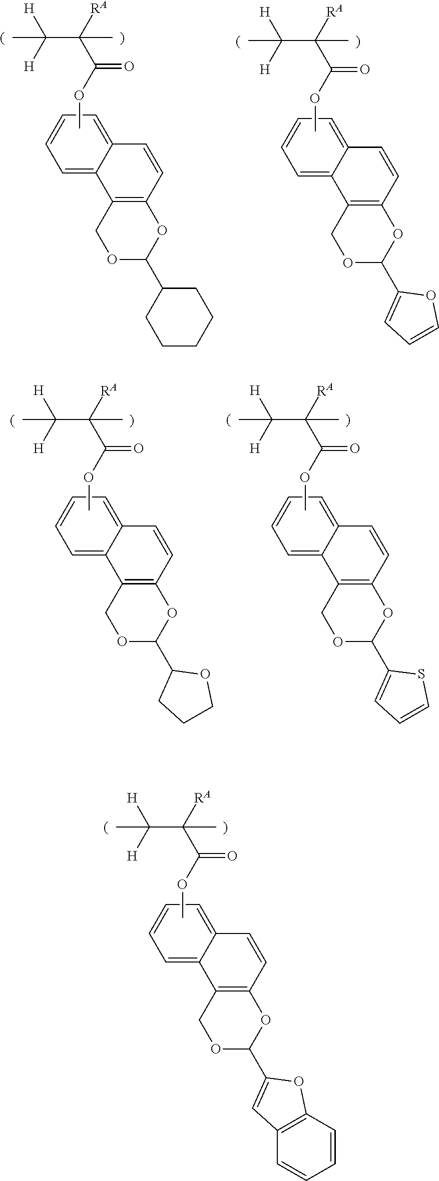

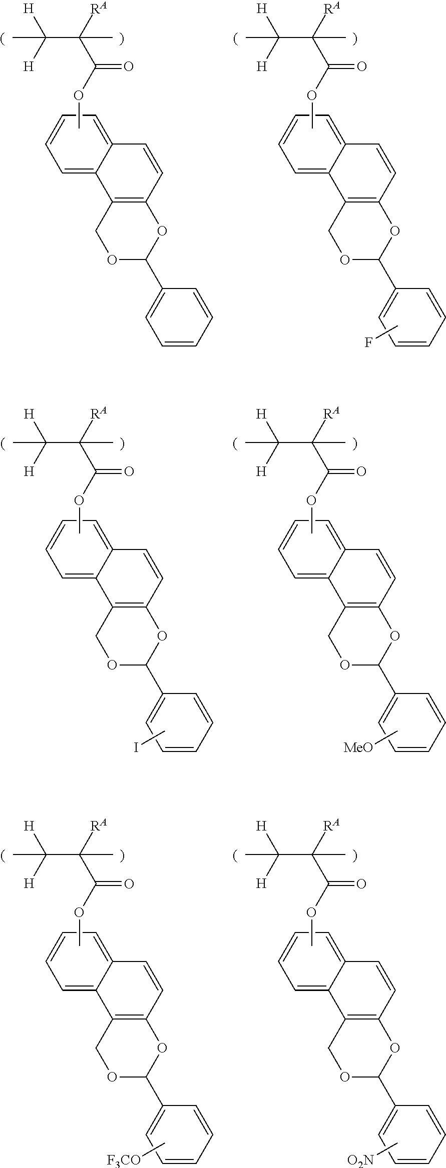

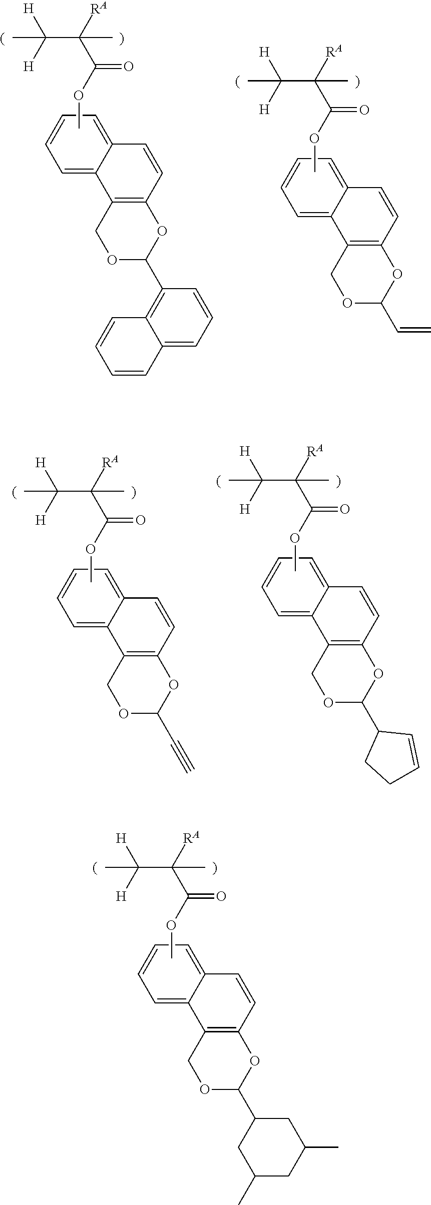

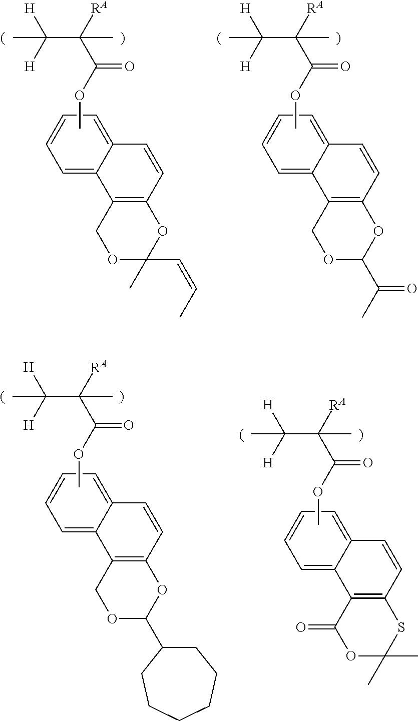

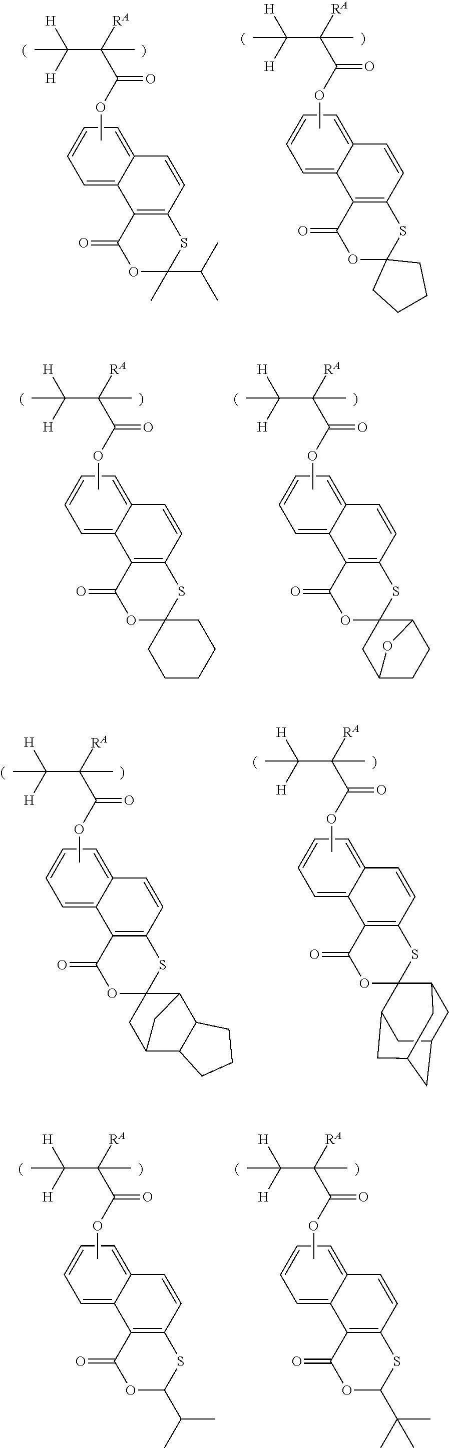

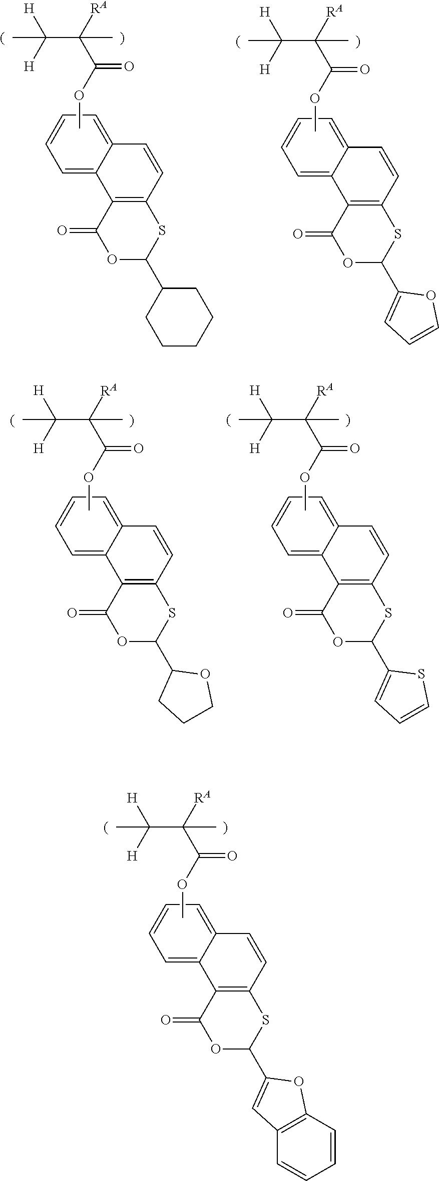

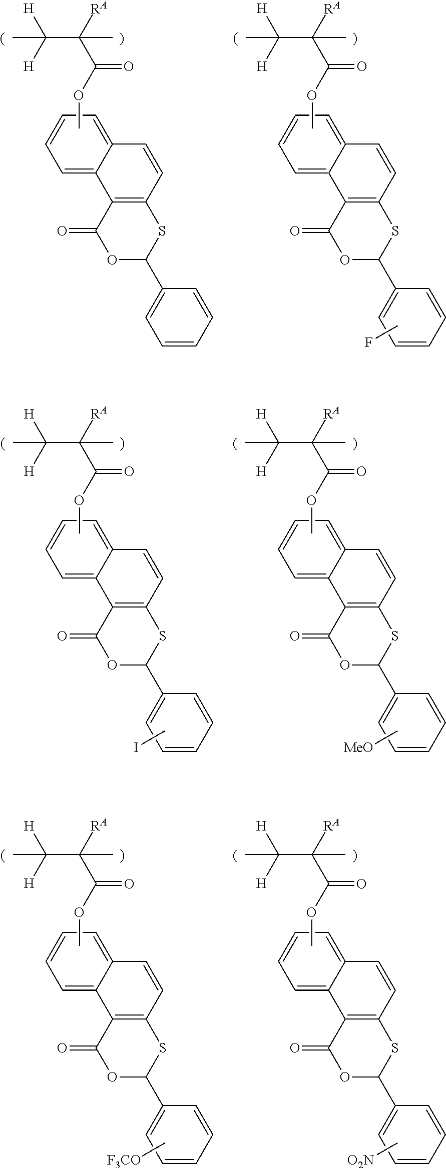

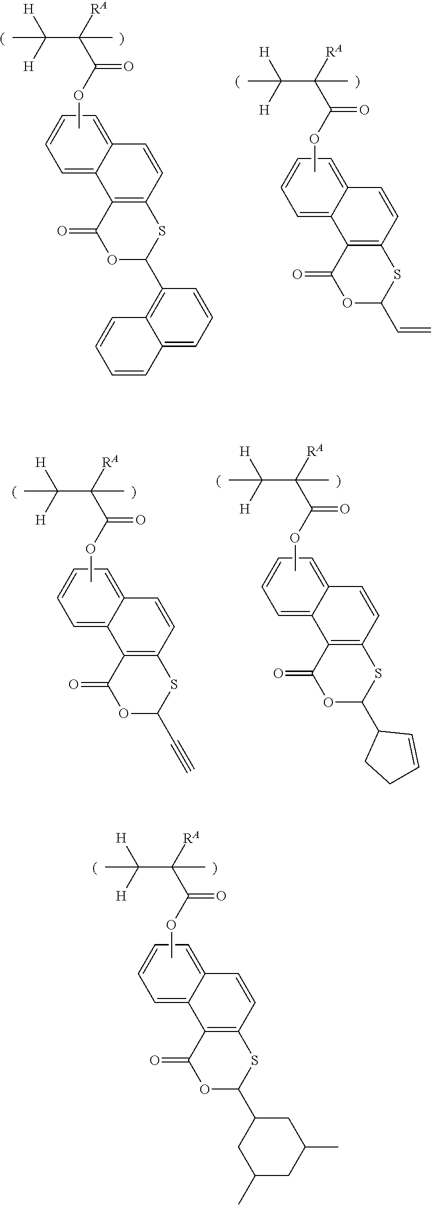

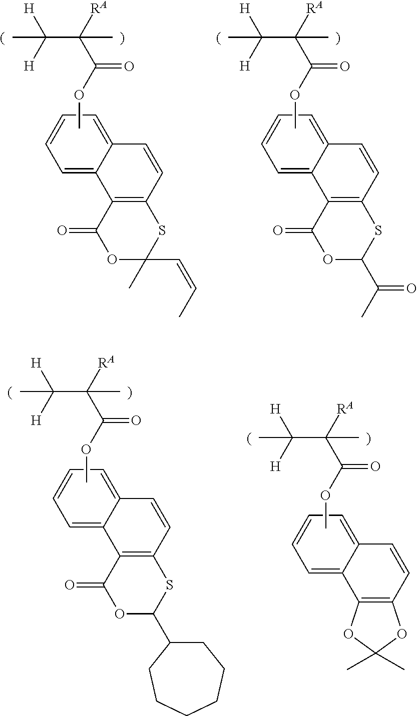

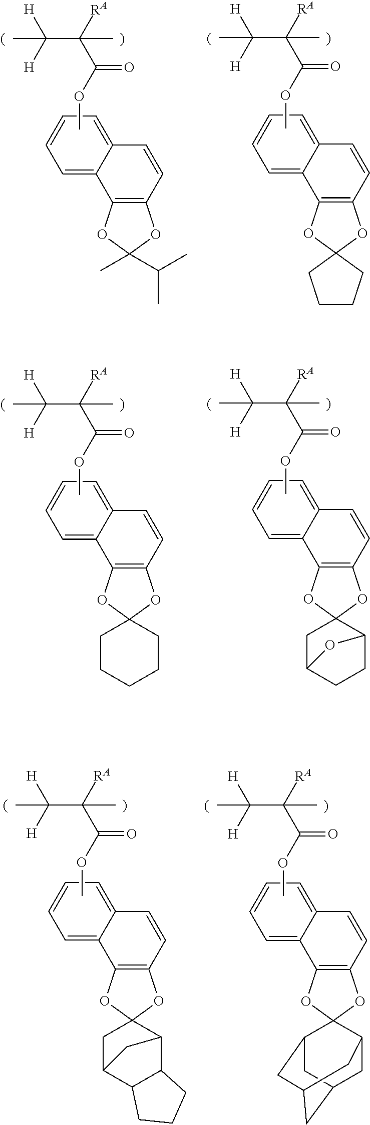

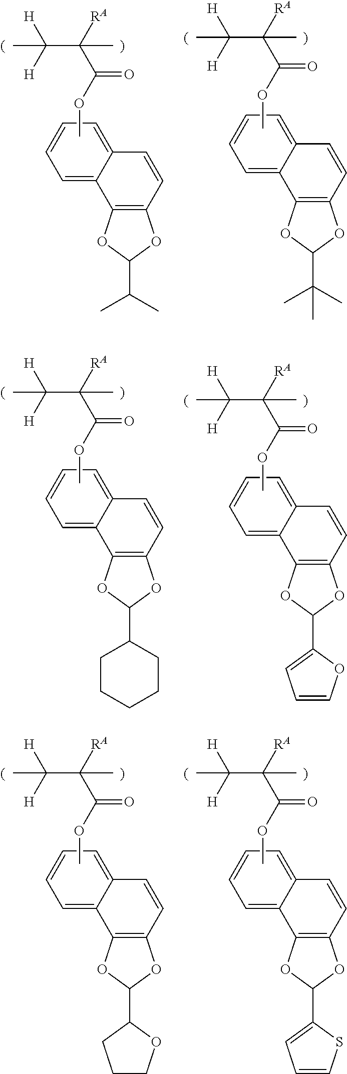

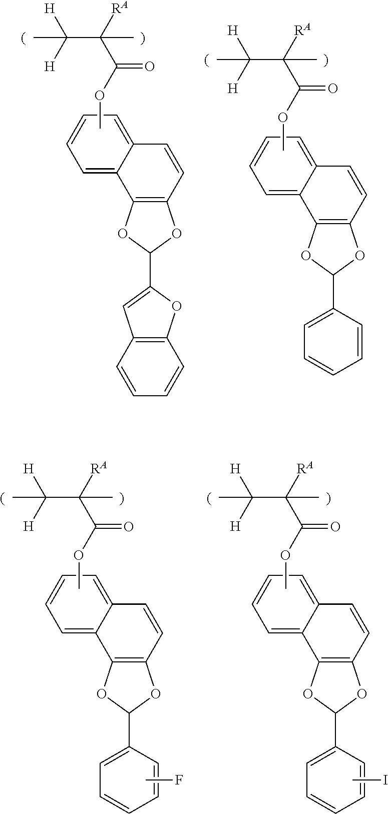

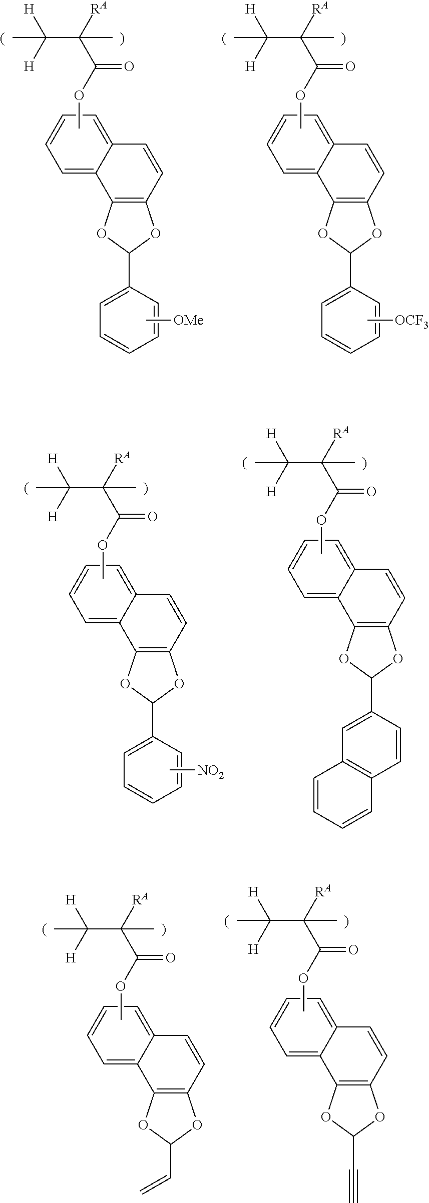

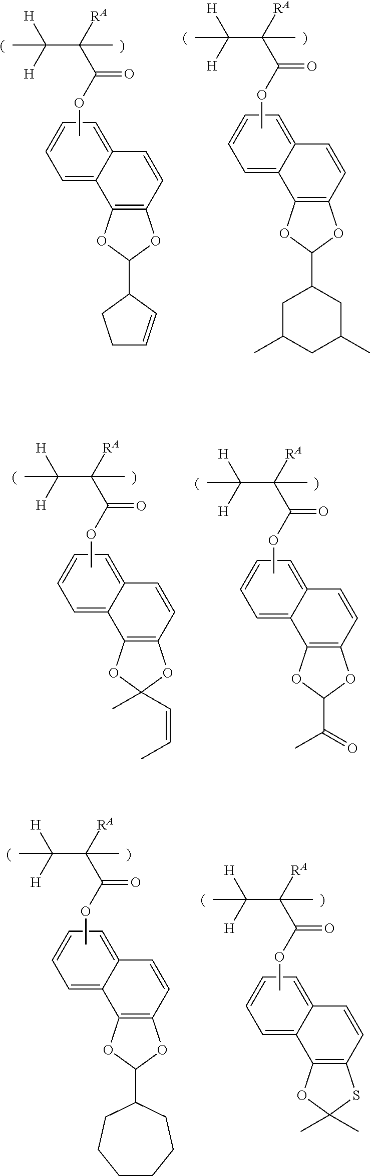

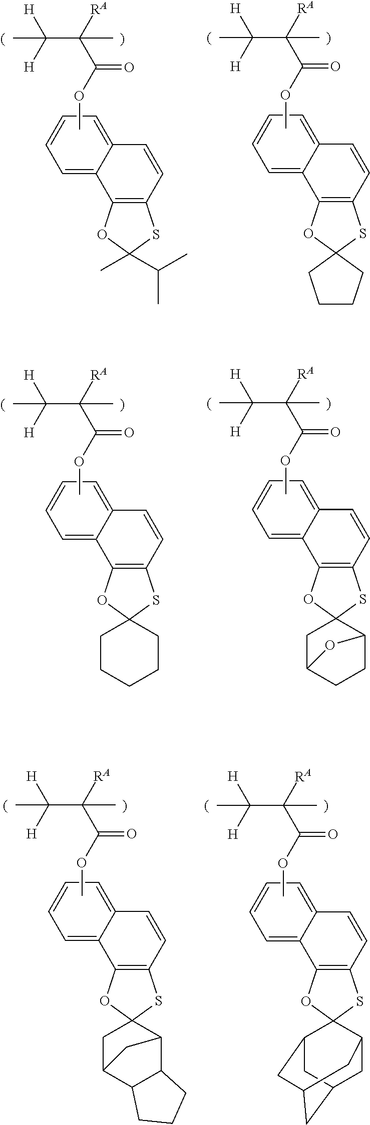

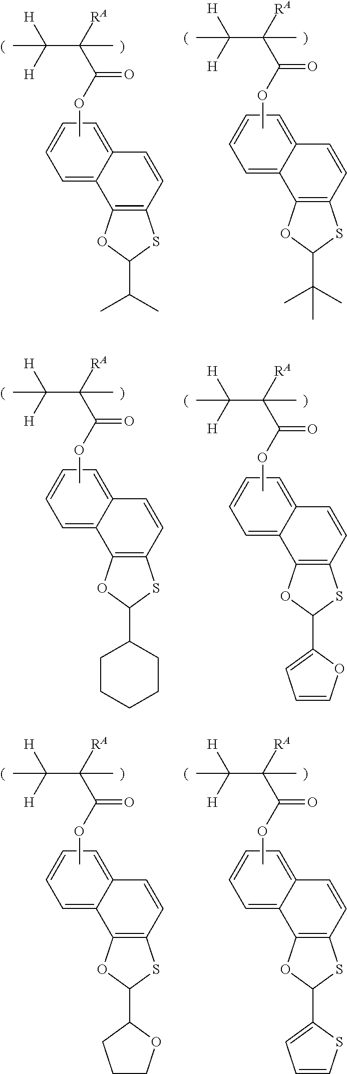

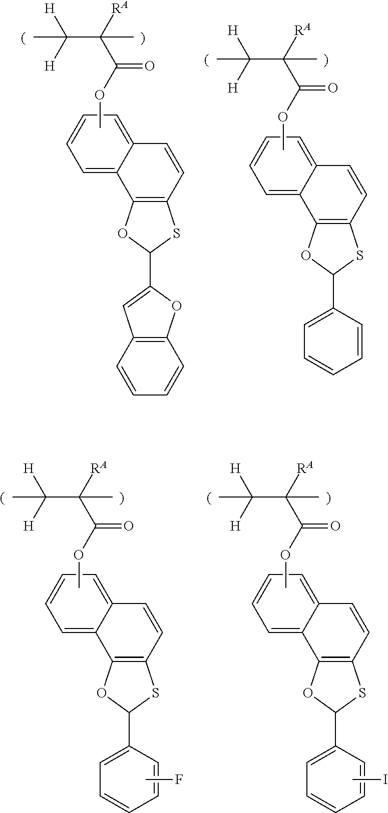

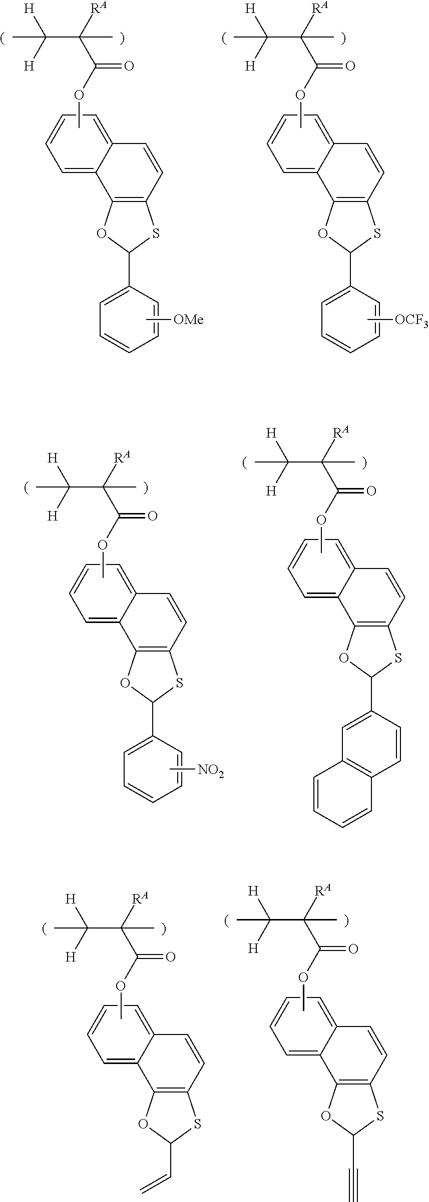

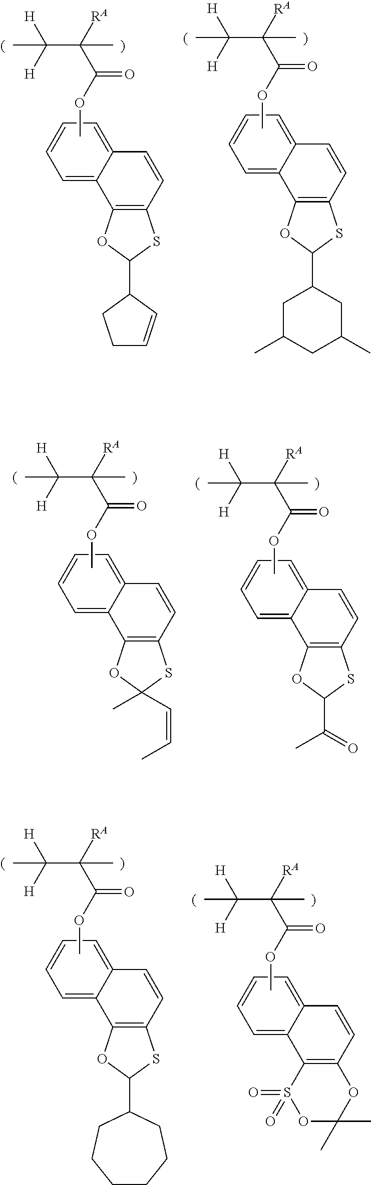

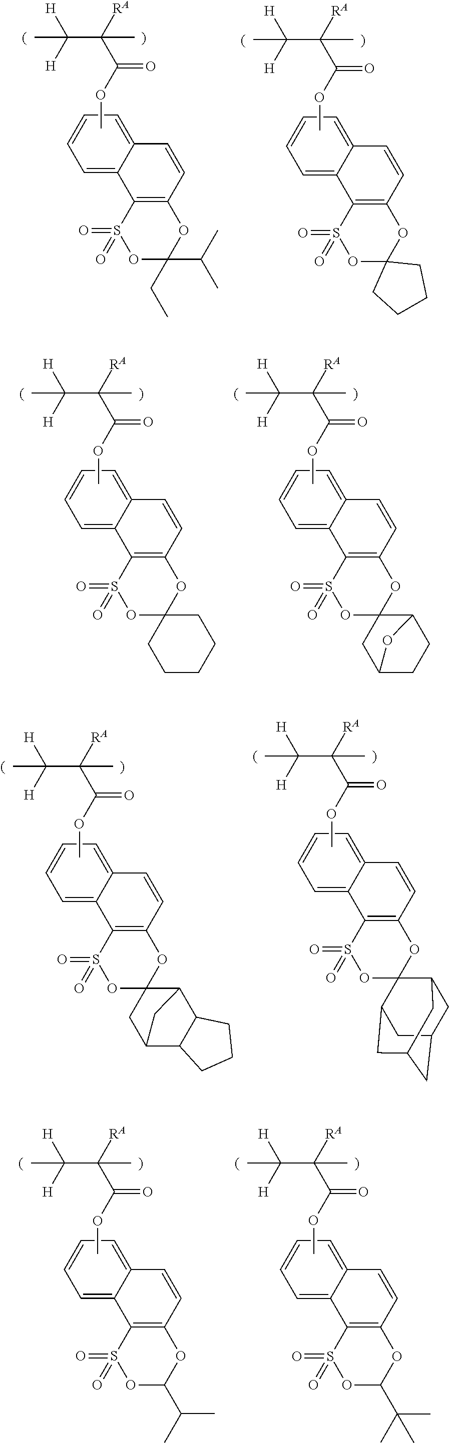

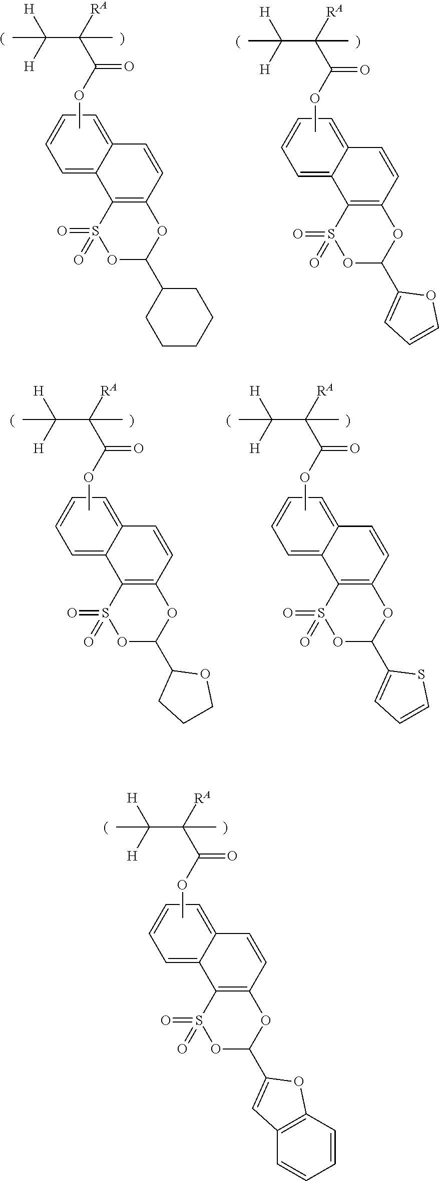

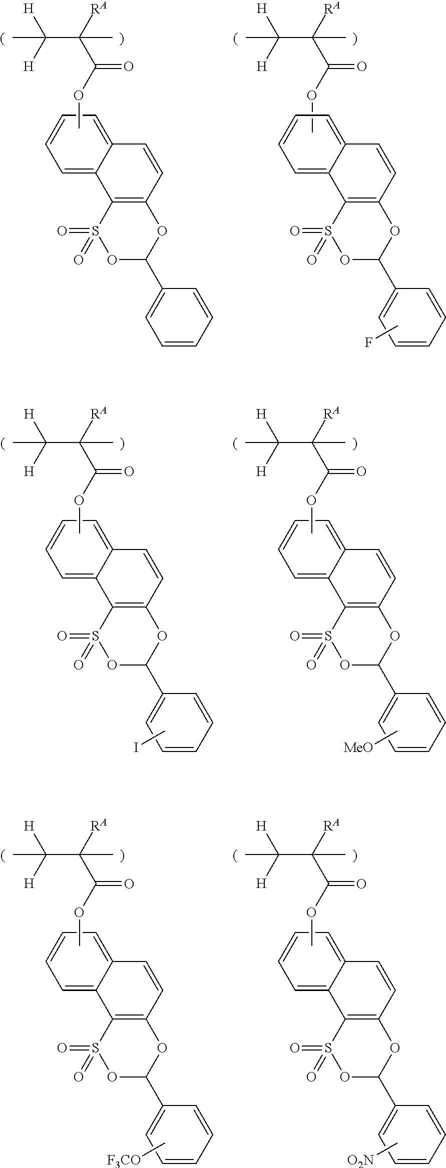

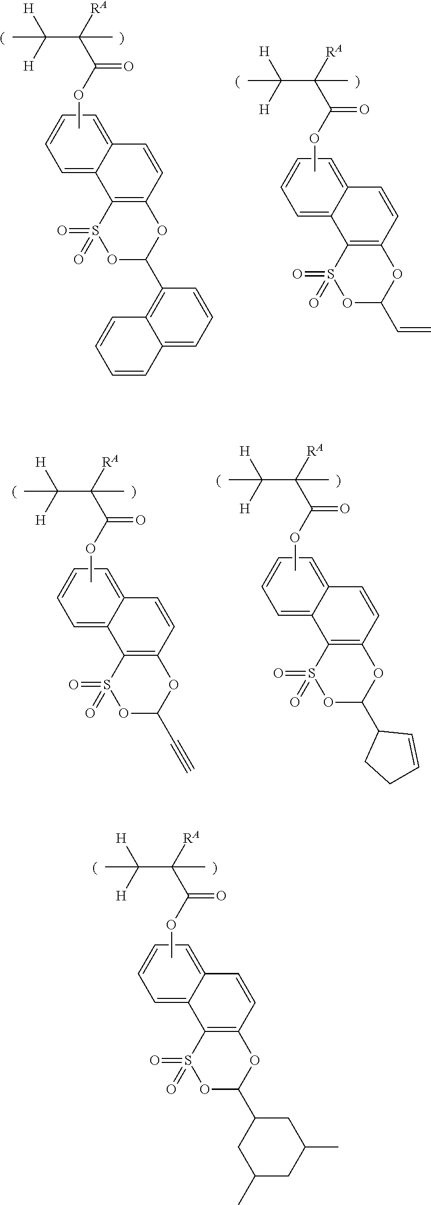

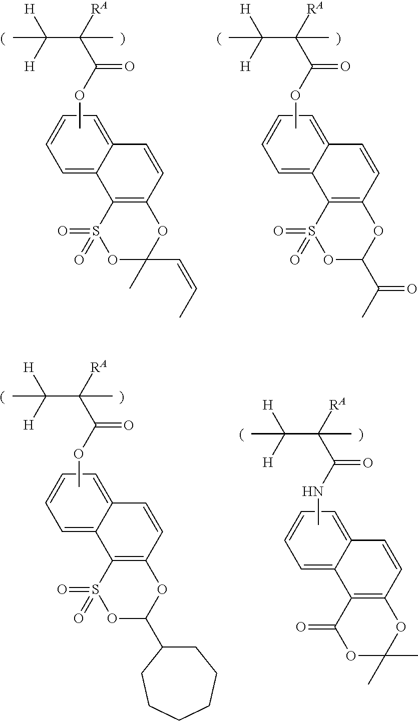

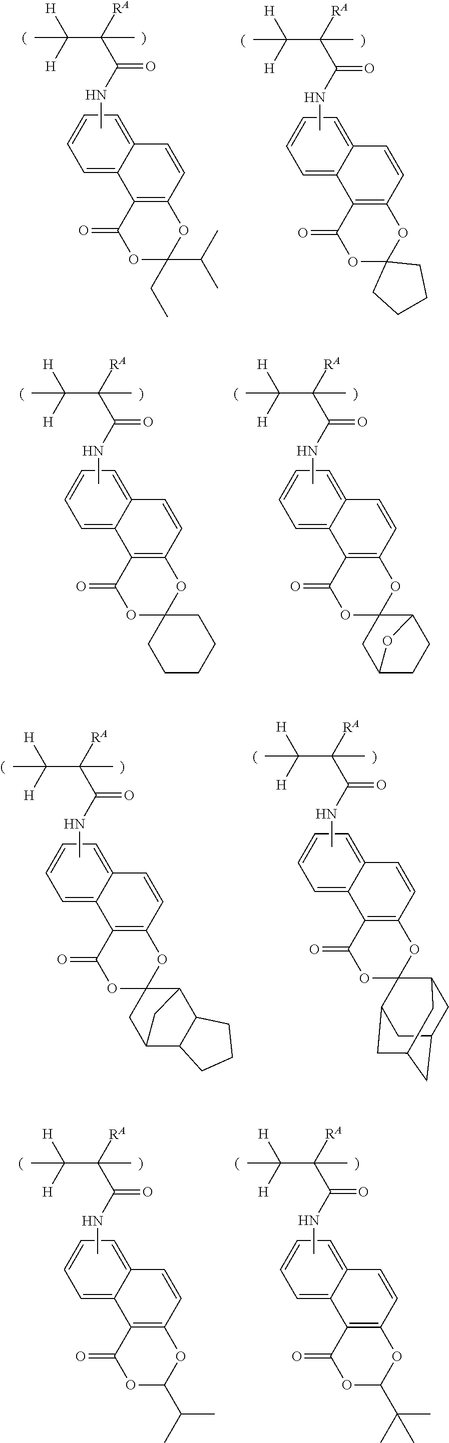

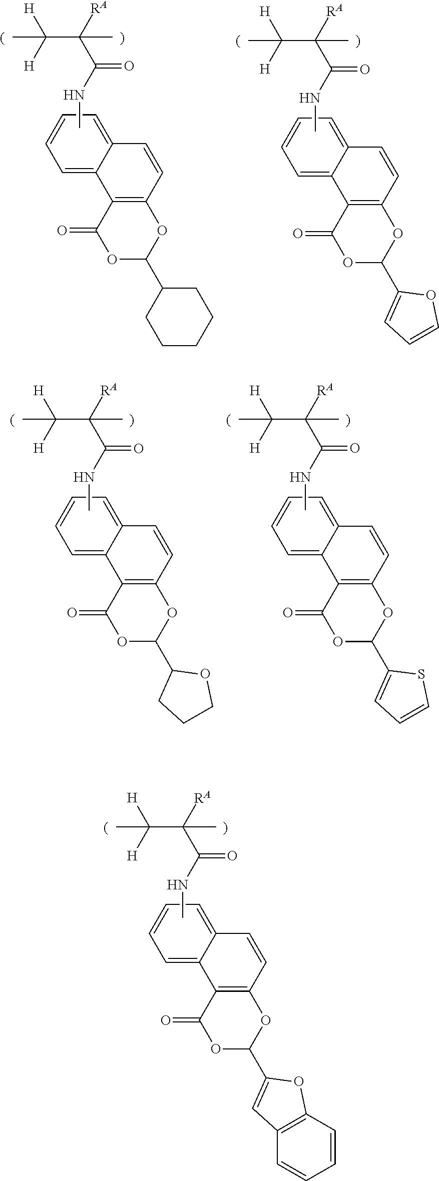

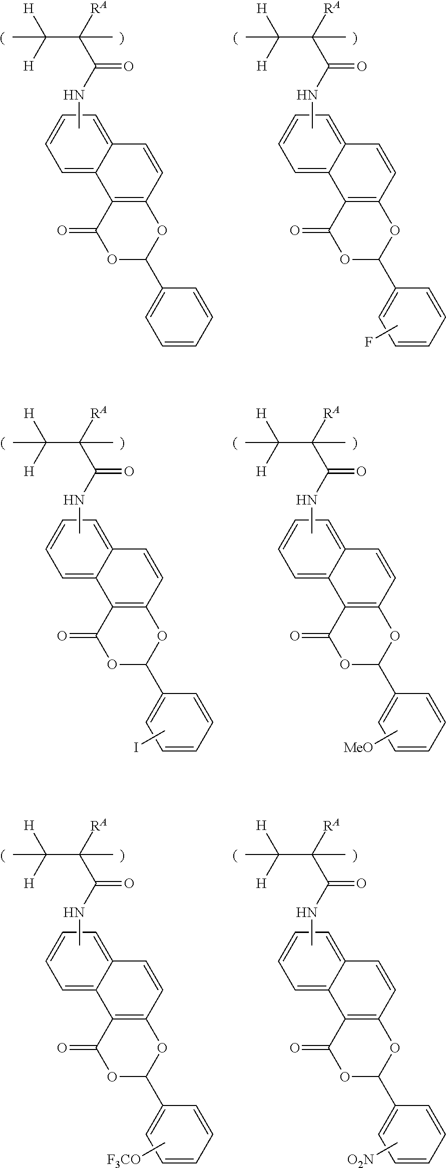

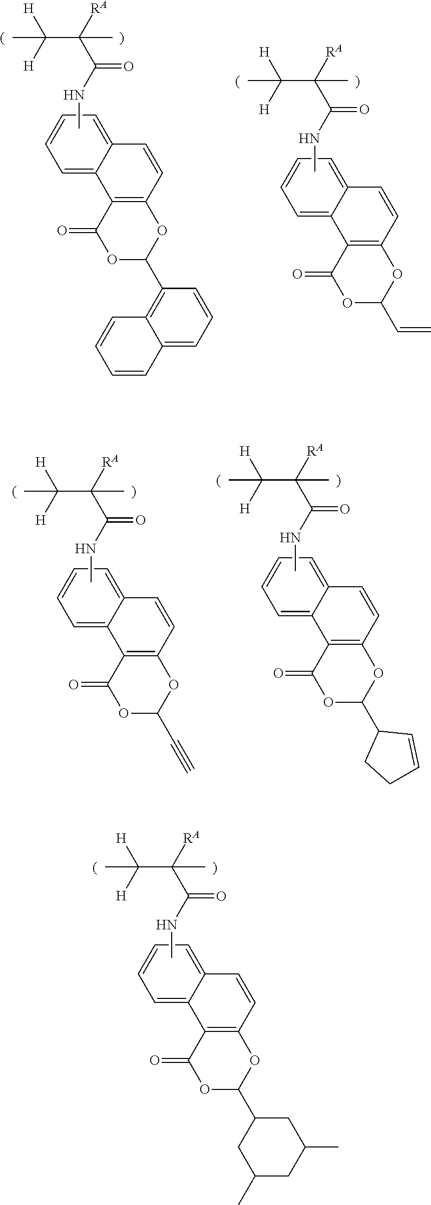

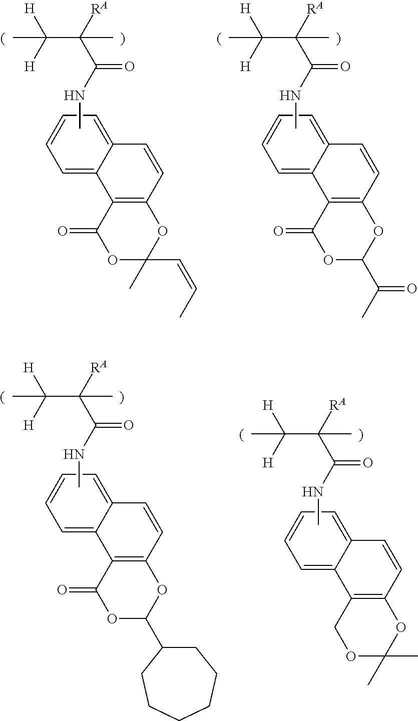

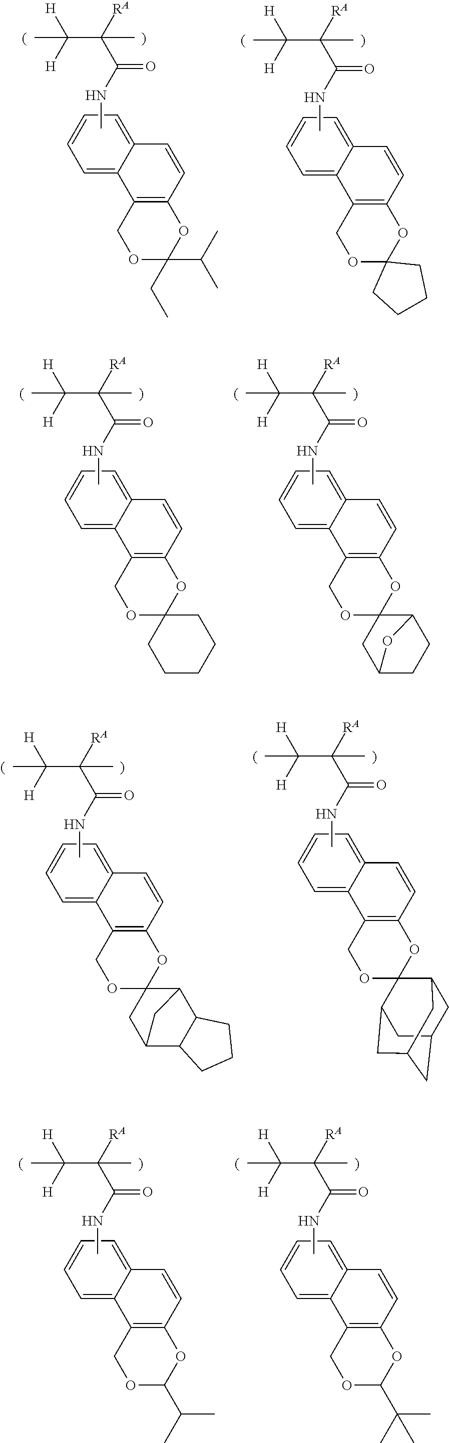

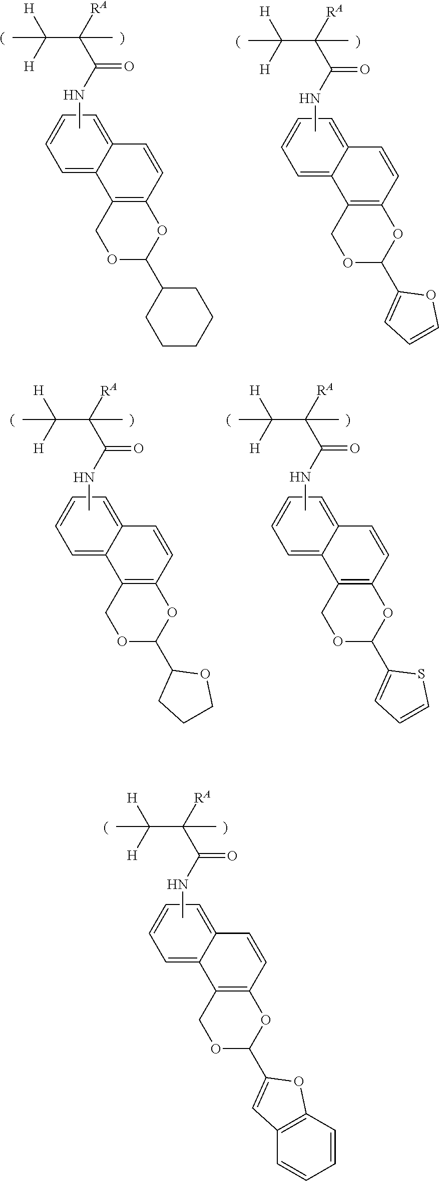

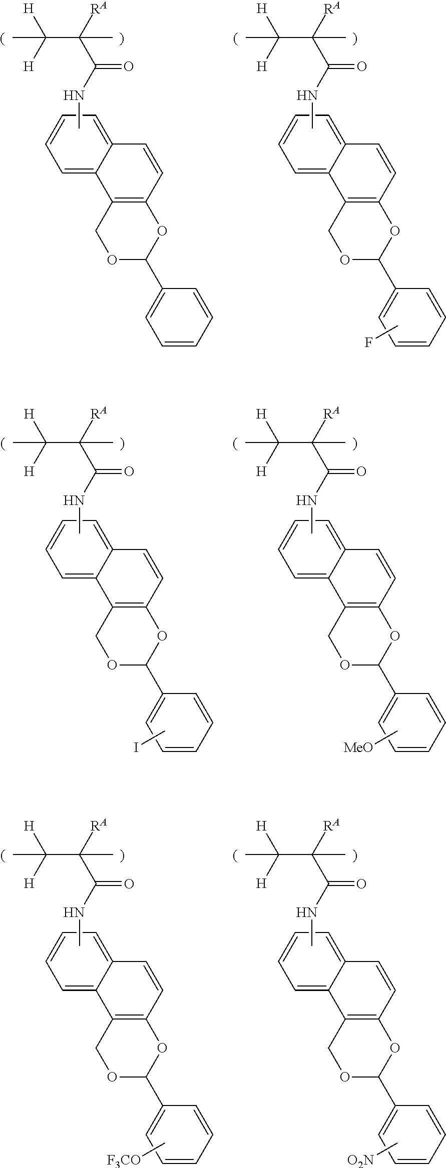

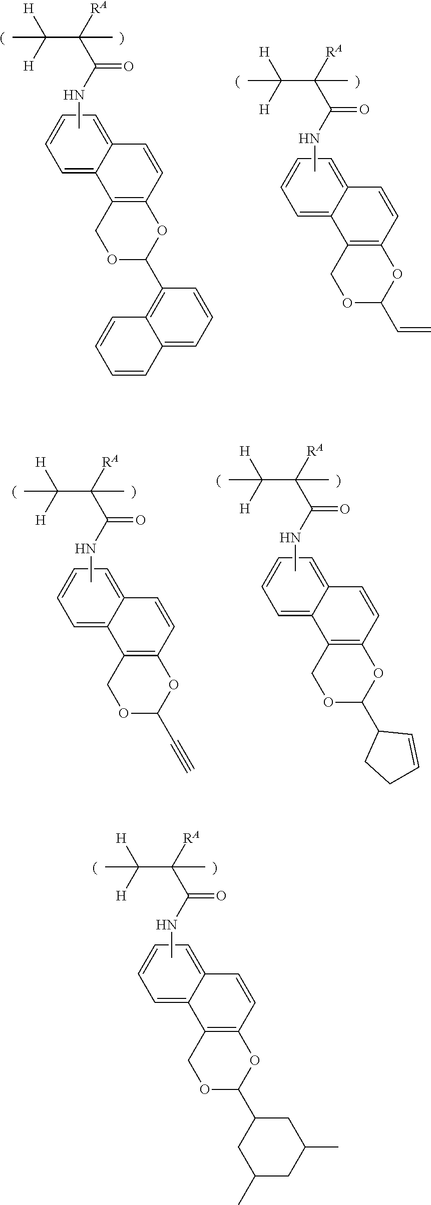

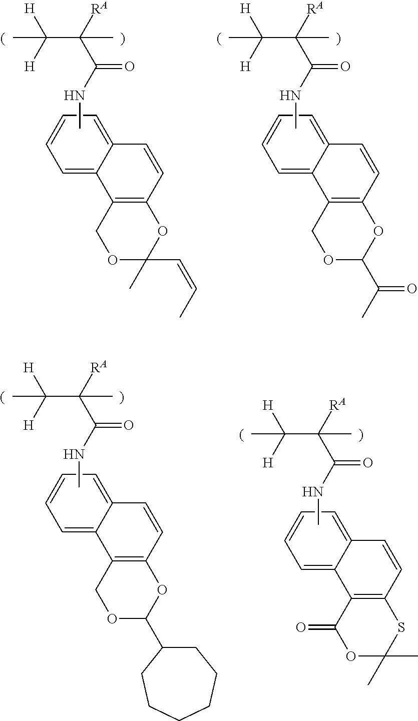

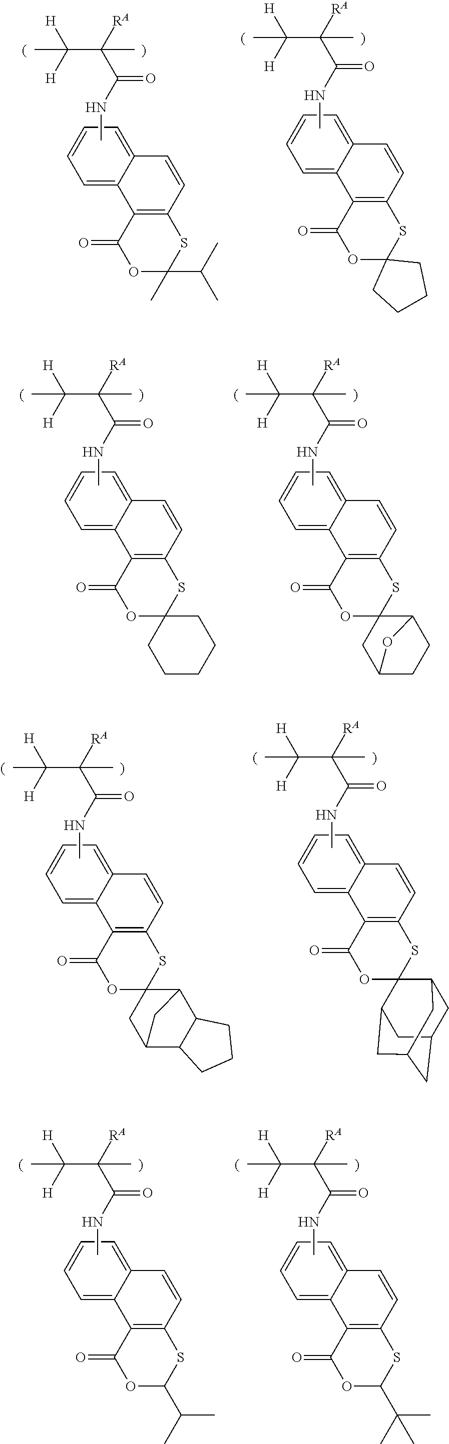

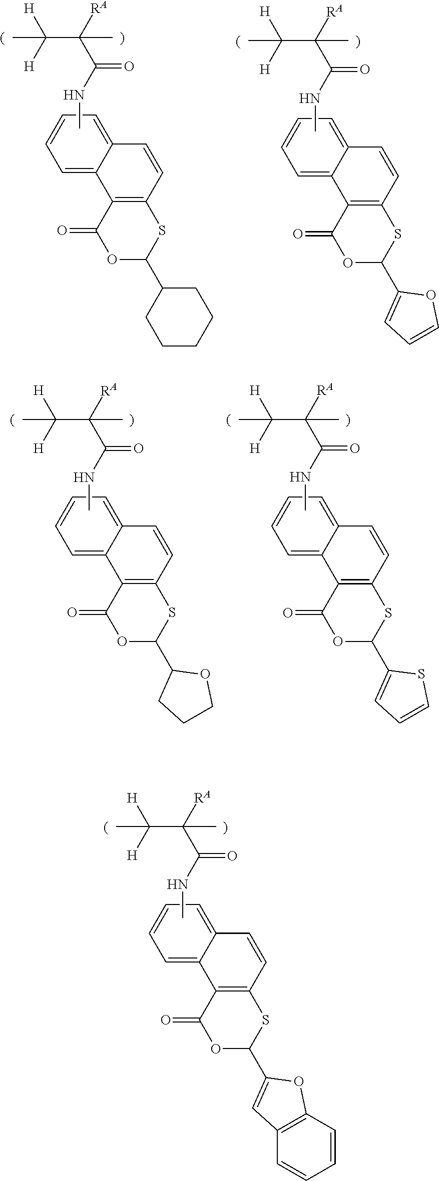

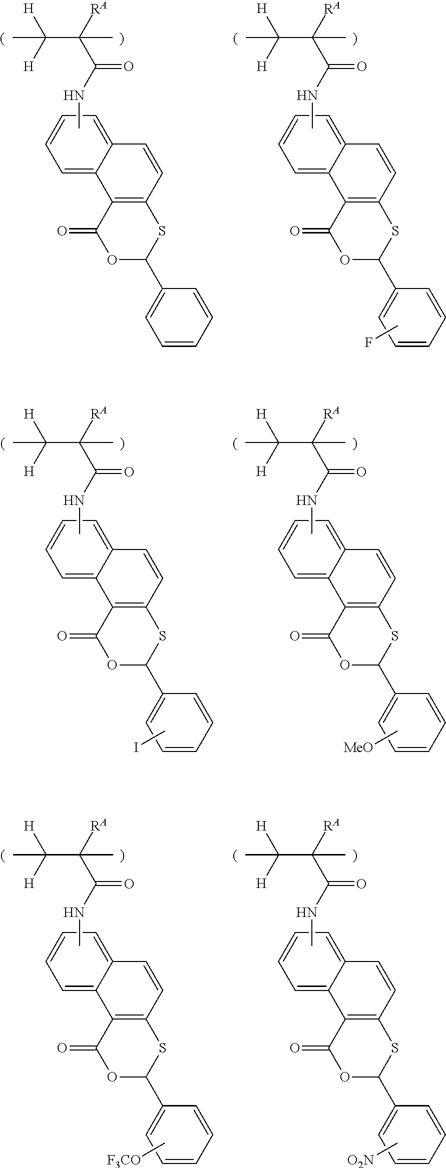

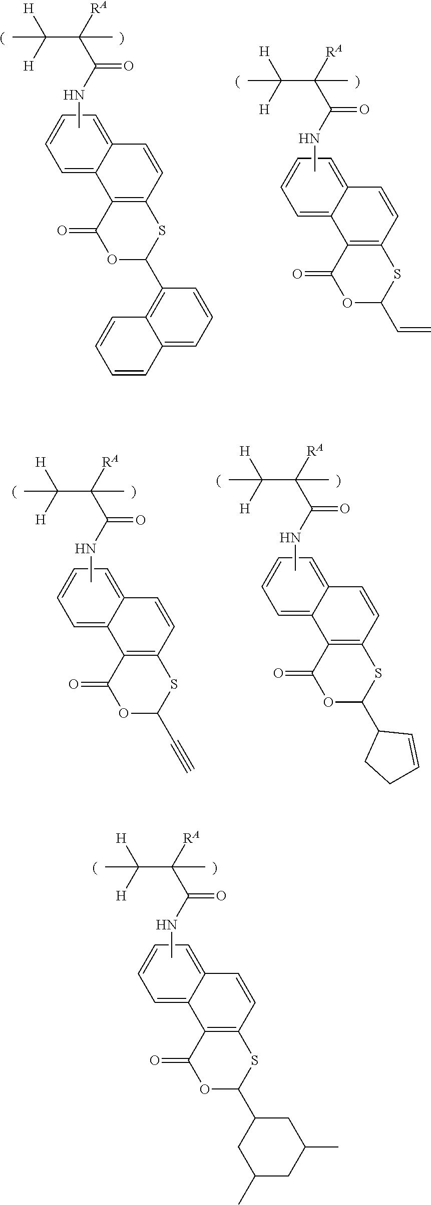

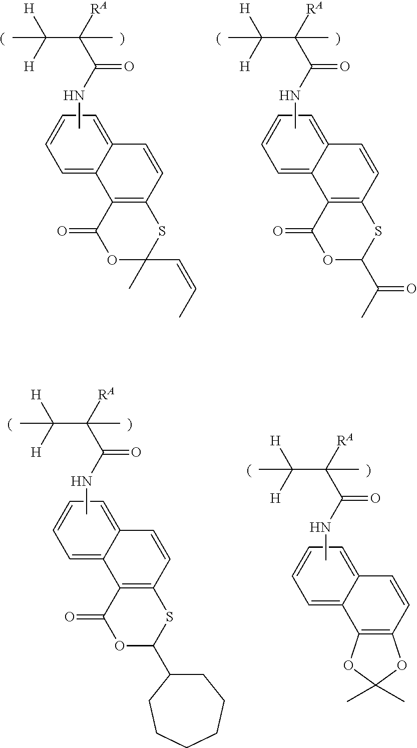

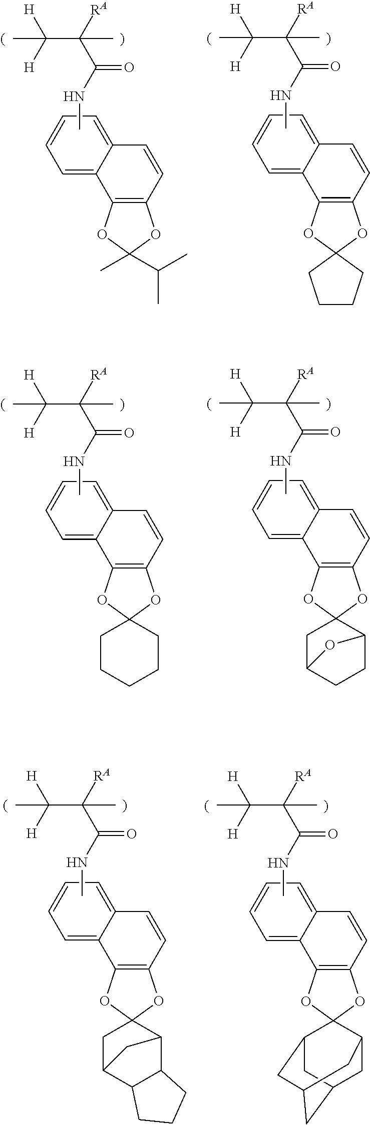

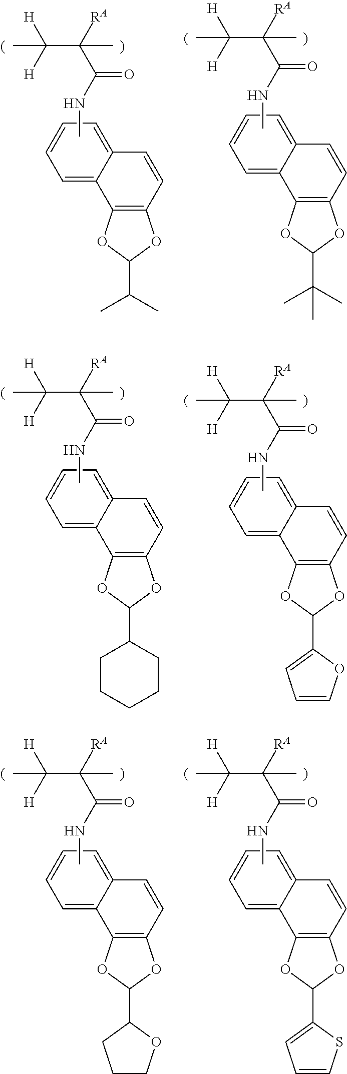

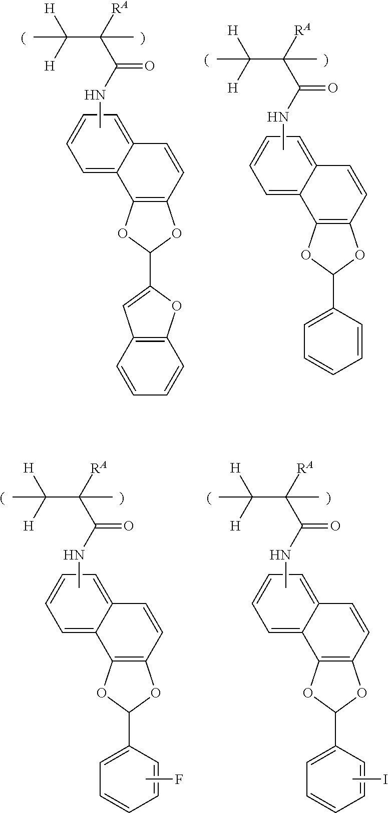

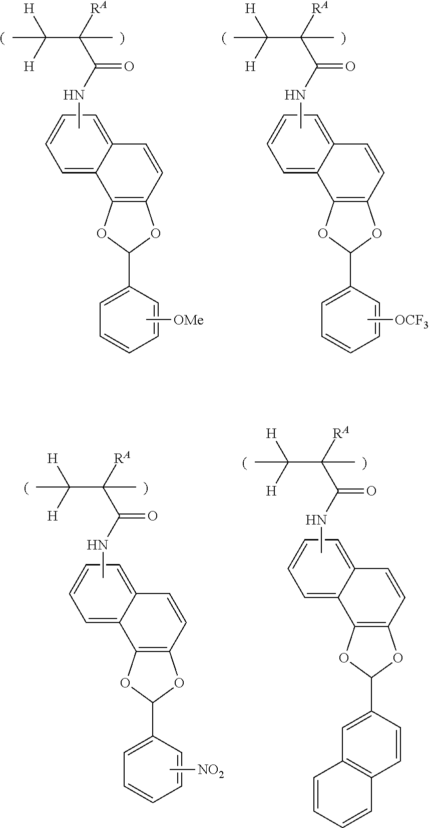

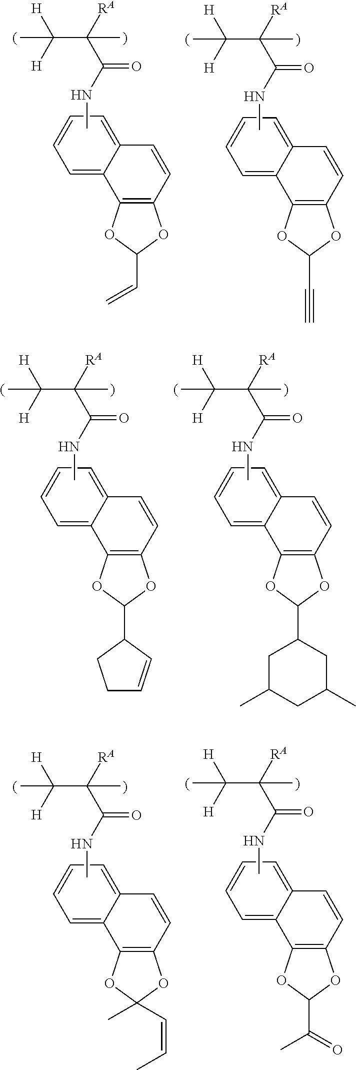

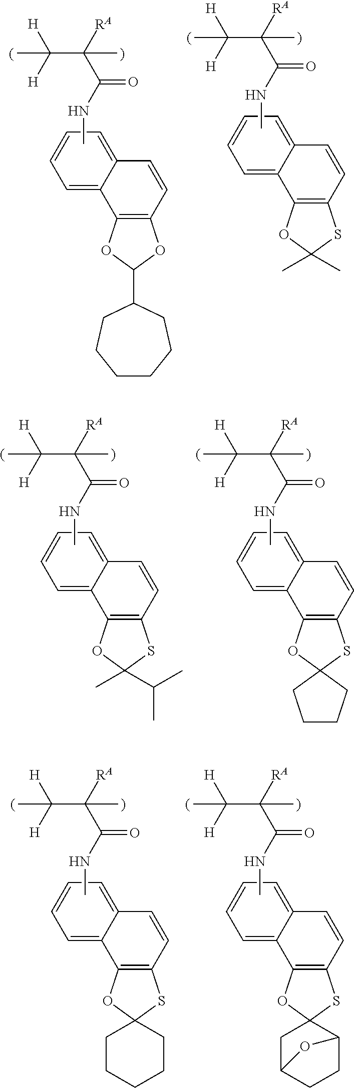

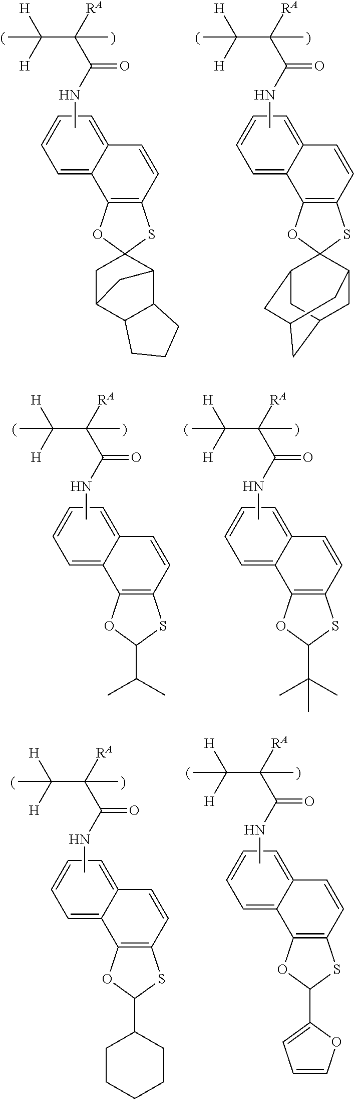

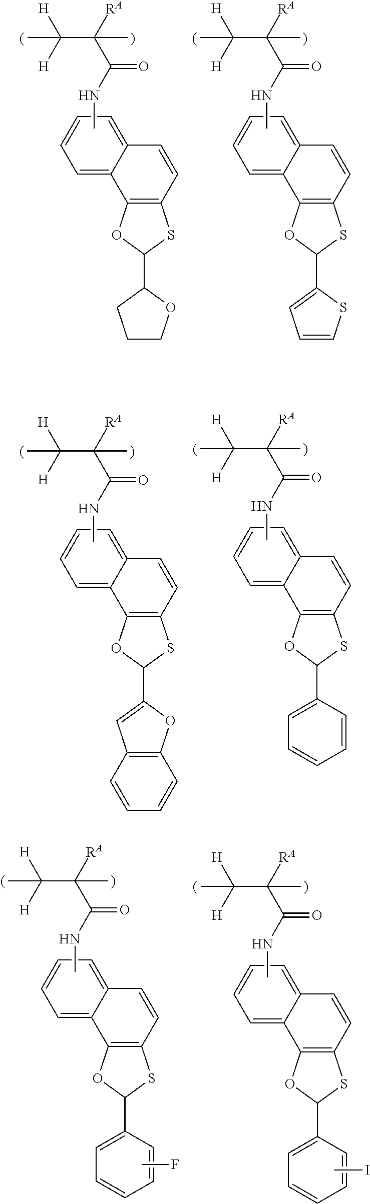

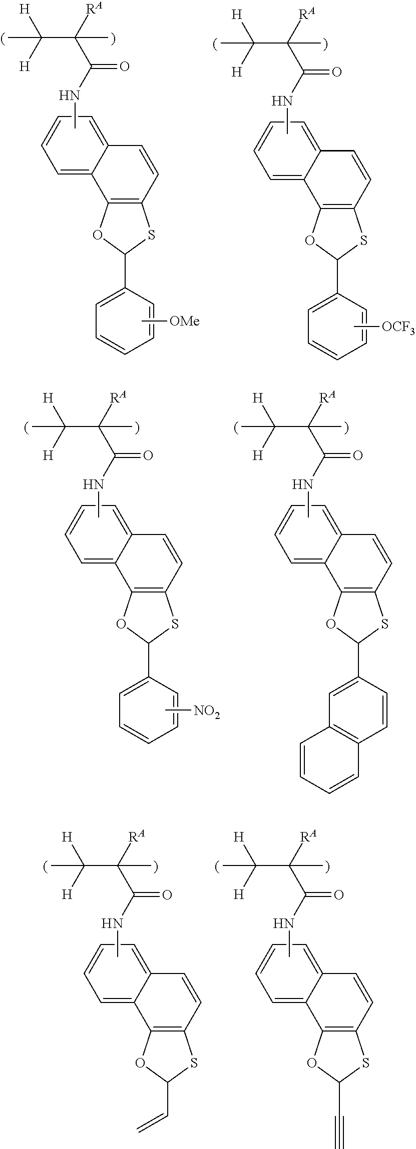

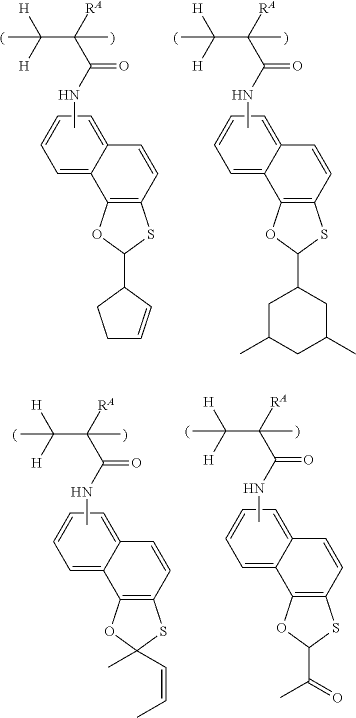

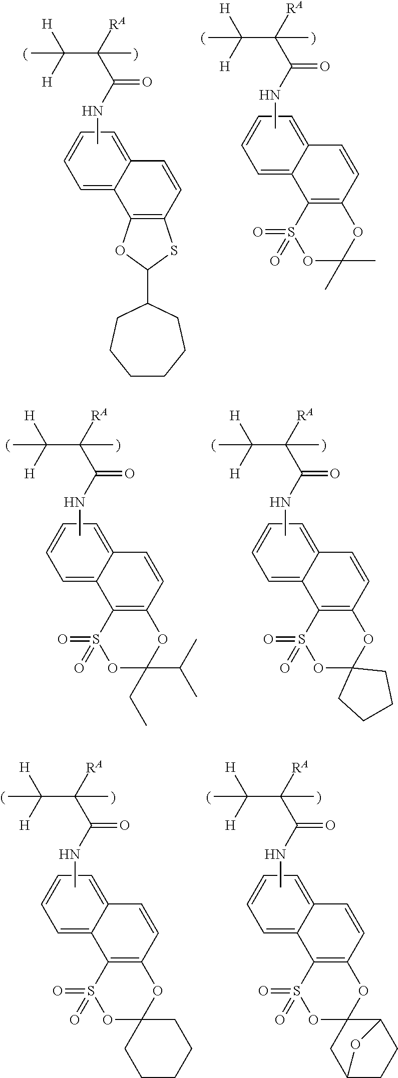

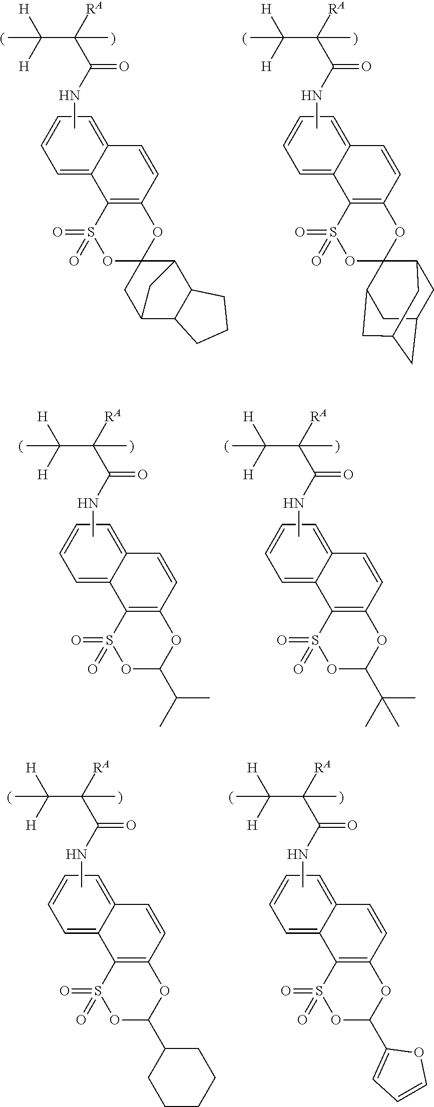

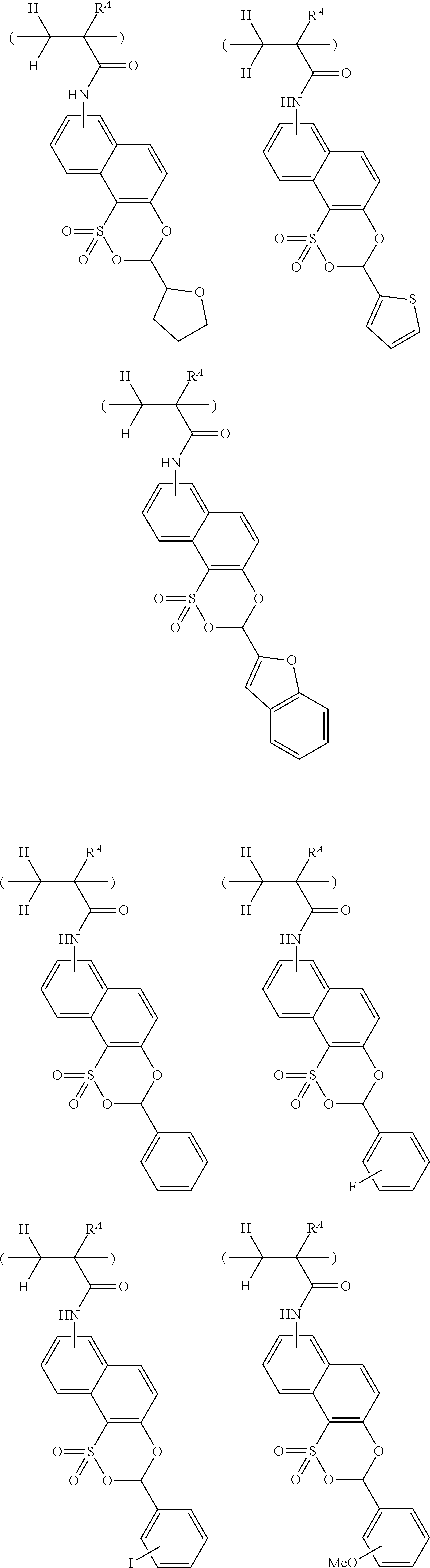

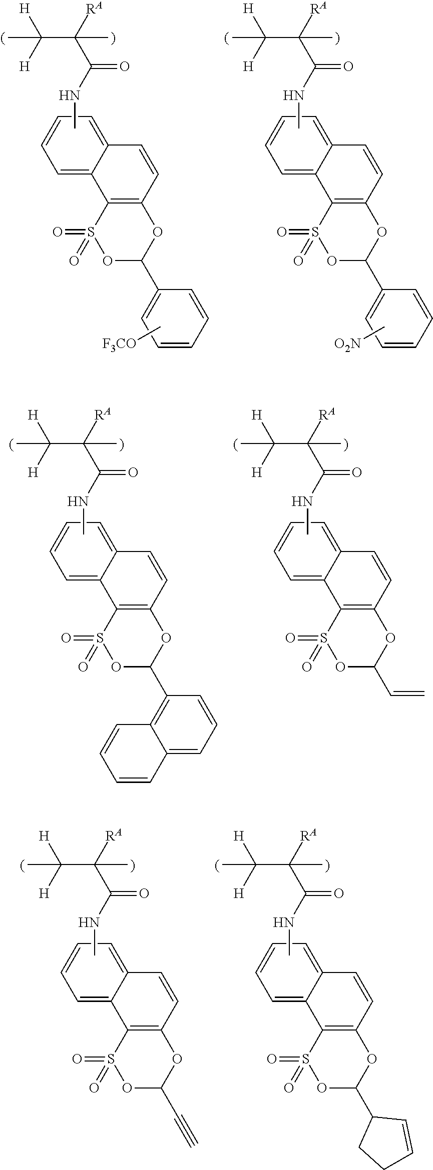

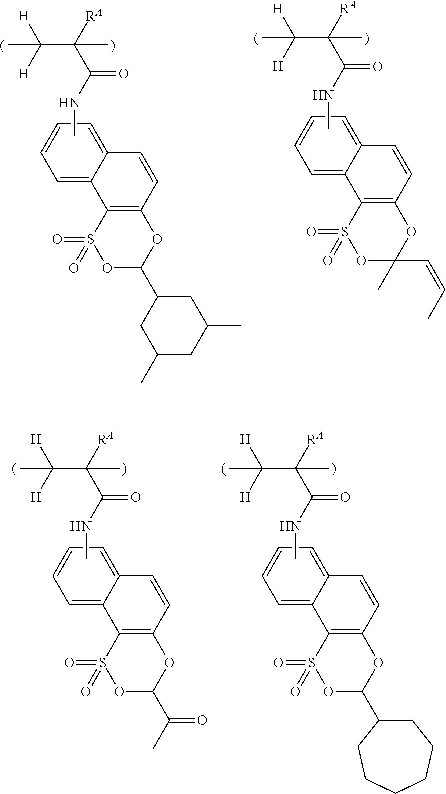

It is preferable that the polymer further contains at least one repeating unit selected from a repeating unit represented by the following general formula (b1) (hereinafter also referred to as “repeating unit-b1”) and a repeating unit represented by the following general formula (b2) (hereinafter also referred to as “repeating unit-b2”).

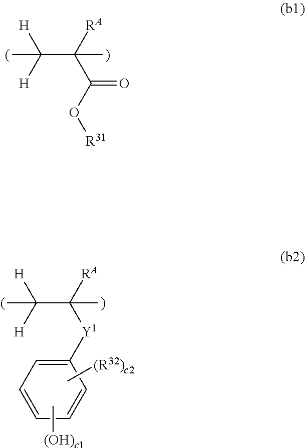

In the formulae, “c1” is an integer from 1 to 4; “c2” represents an integer from 0 to 4; and 1≤c1+c2≤5;
R^{A} each independently represents a hydrogen atom, a fluorine atom, a methyl group, or a trifluoromethyl group;
Y¹ represents a single bond or *—C(═O)—O—; “*” represents an attachment point to a main chain carbon atom;
R³¹ represents a hydrogen atom, or a group having 1 to 20 carbon atoms and containing at least one structure selected from the group consisting of a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—);
R³² represents a halogen atom, a carboxy group, a nitro group, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and when “c2” represents 2 or more, each R³² may be the same as or different from each other.

In the general formulae (b1) and (b2), R^{A} each independently represents a hydrogen atom, a fluorine atom, a methyl group, or a trifluoromethyl group. Y¹ represents a single bond or *—C(═O)—O—. “*” represents an attachment point to a main chain carbon atom. R³¹ represents a hydrogen atom, or a group having 1 to 20 carbon atoms and containing at least one structure selected from the group consisting of a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—). R³² represents a halogen atom, a carboxy group, a nitro group, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom. “c1” represents an integer from 1 to 4. “c2” represents an integer from 0 to 4. Note, 1≤c1+c2≤5. When “c2” is 2 or more, each R³² may be the same as or different from each other.

Specific examples of the repeating unit-b1 include, but are not limited to, the following. In the following formulae, R^{A} is as defined above.

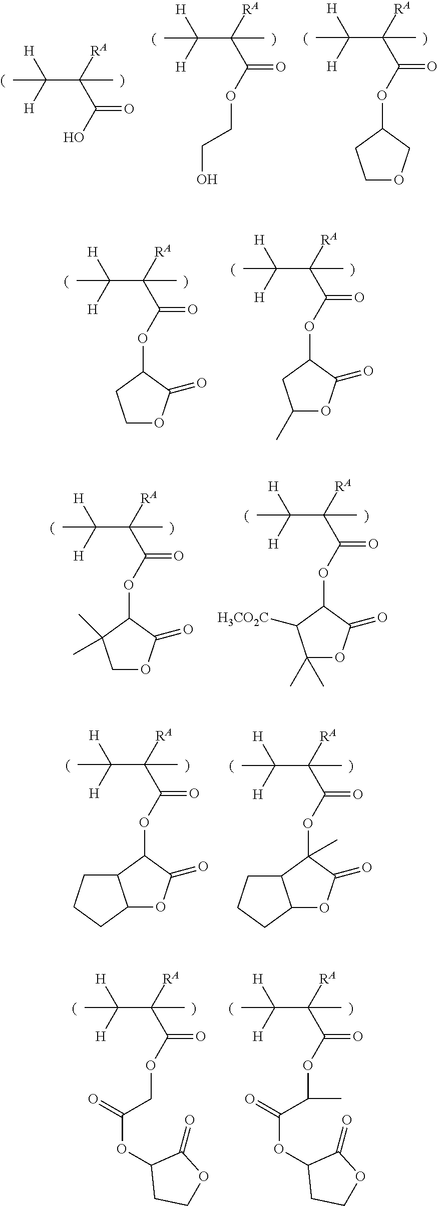

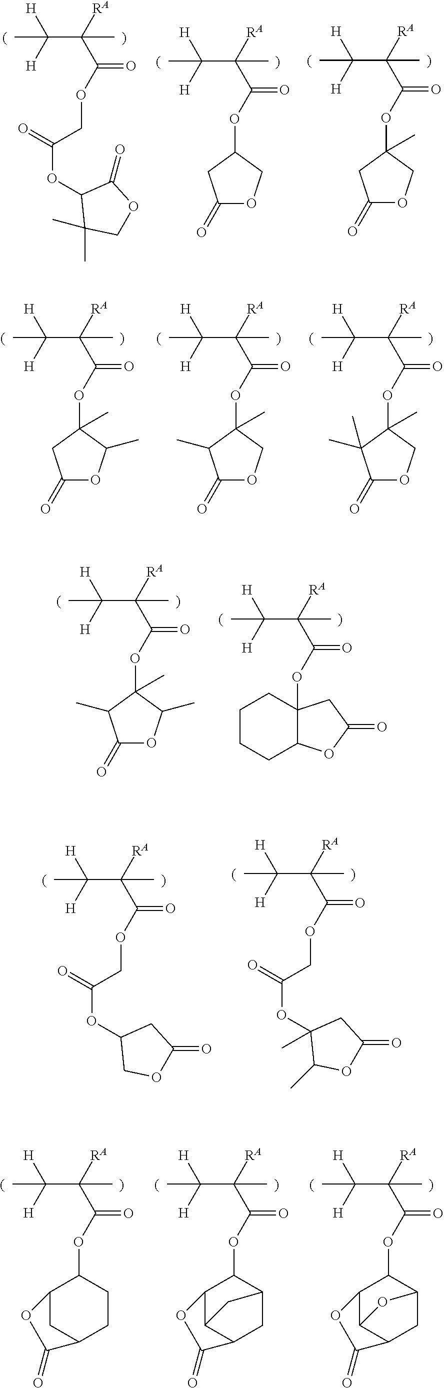

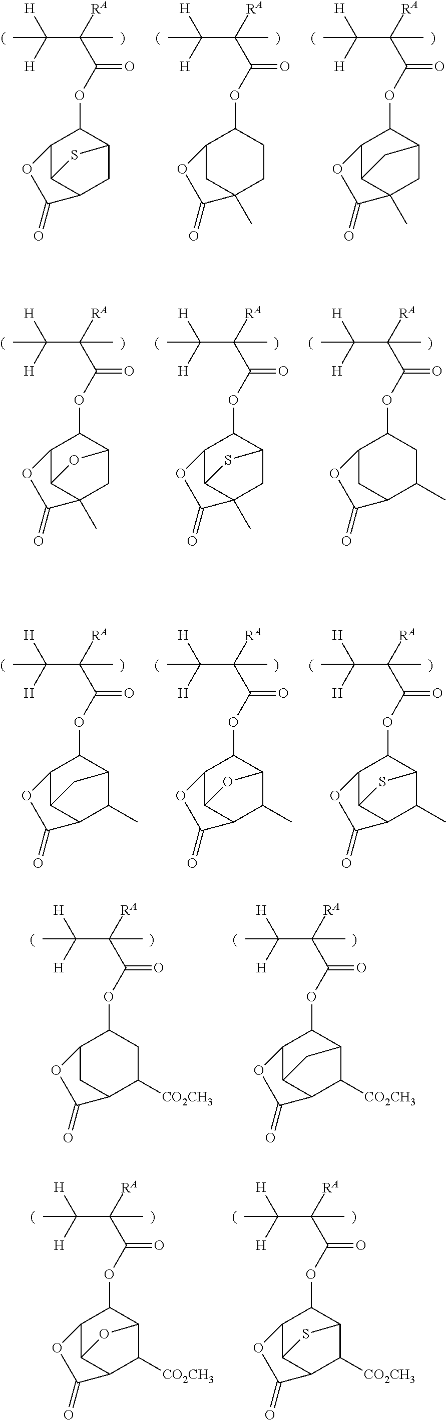

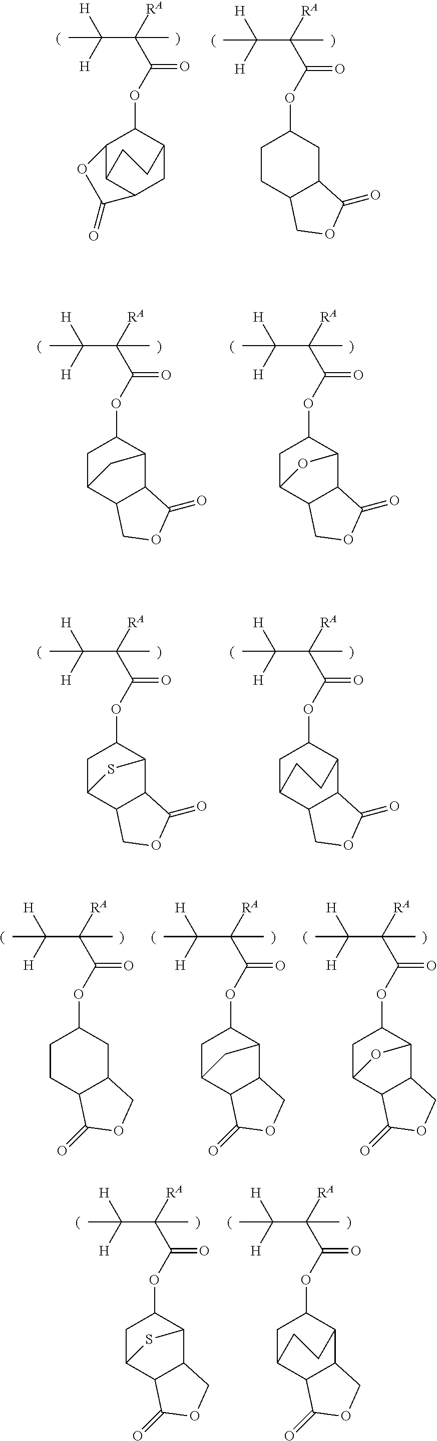

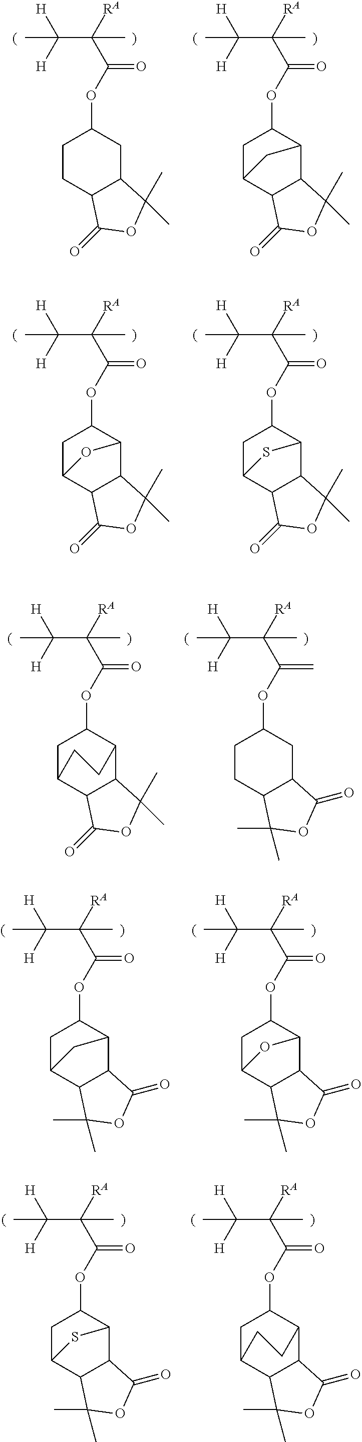

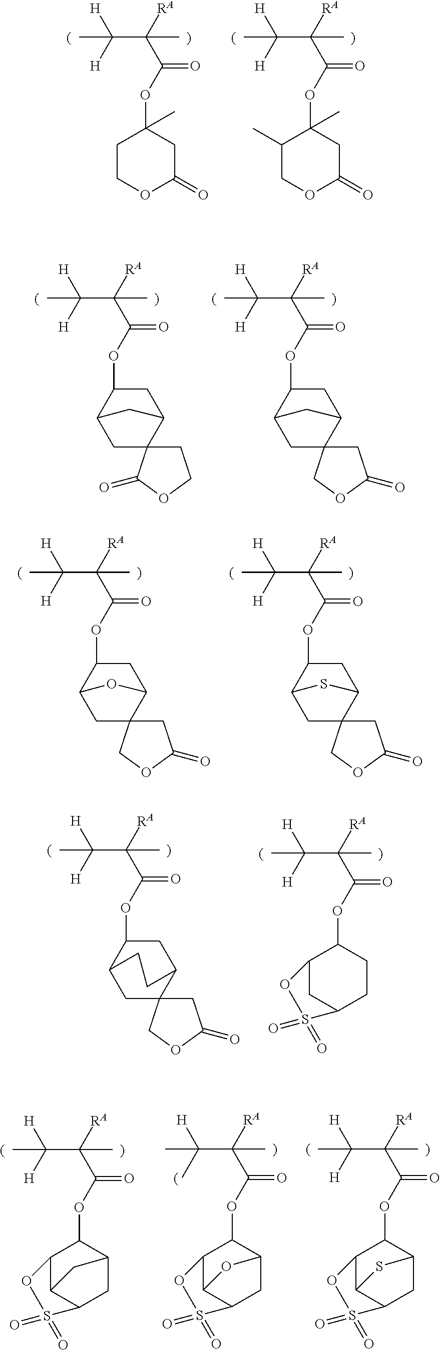

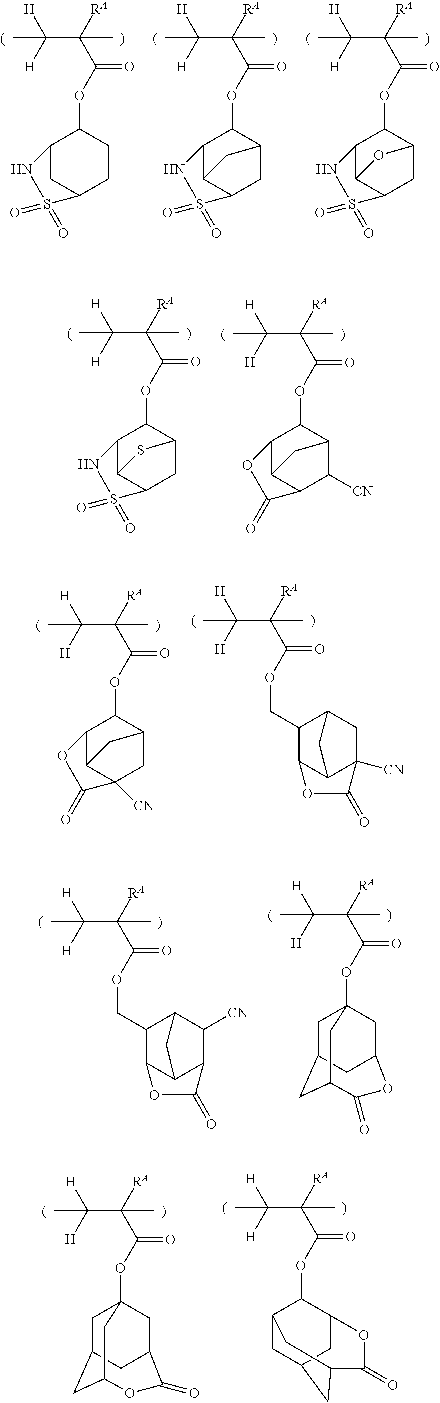

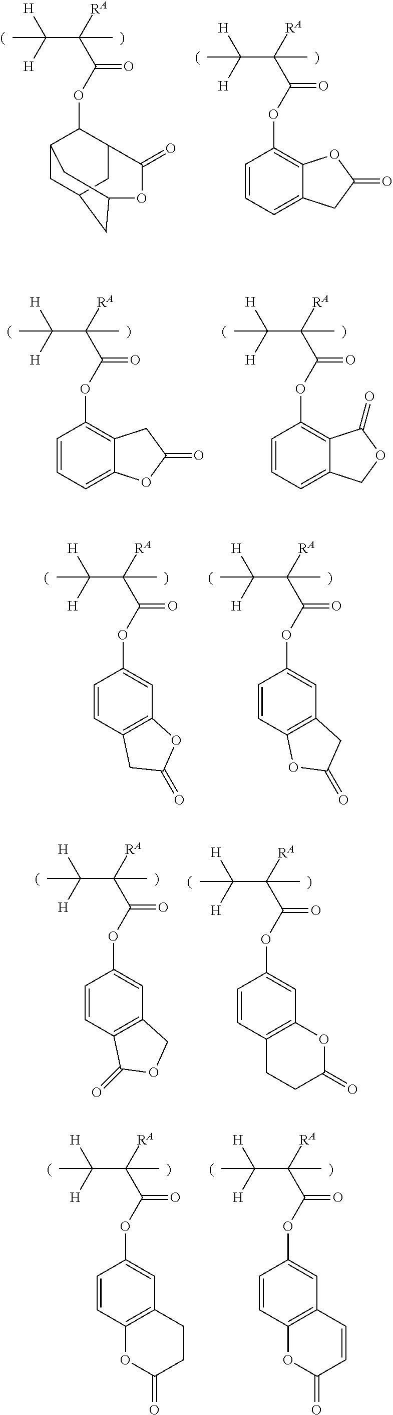

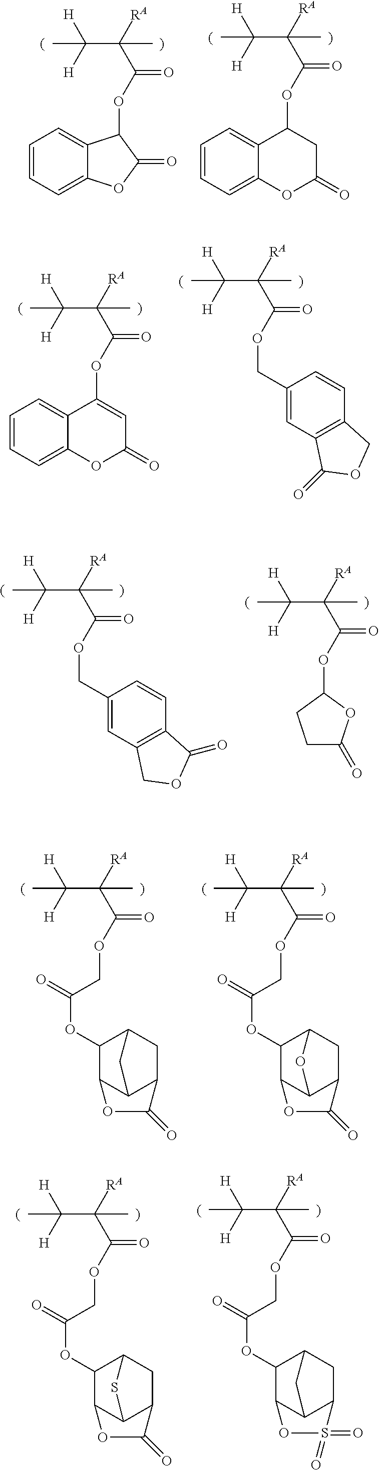

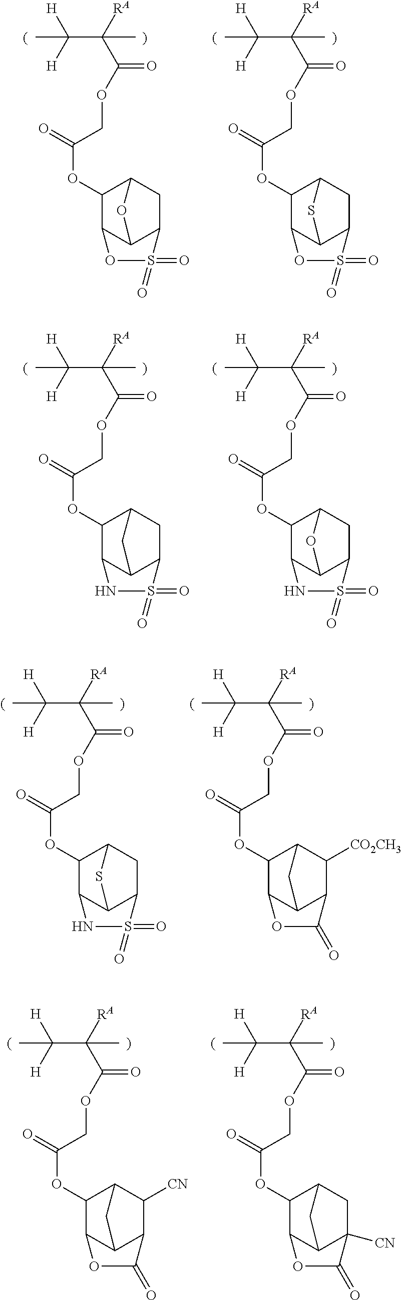

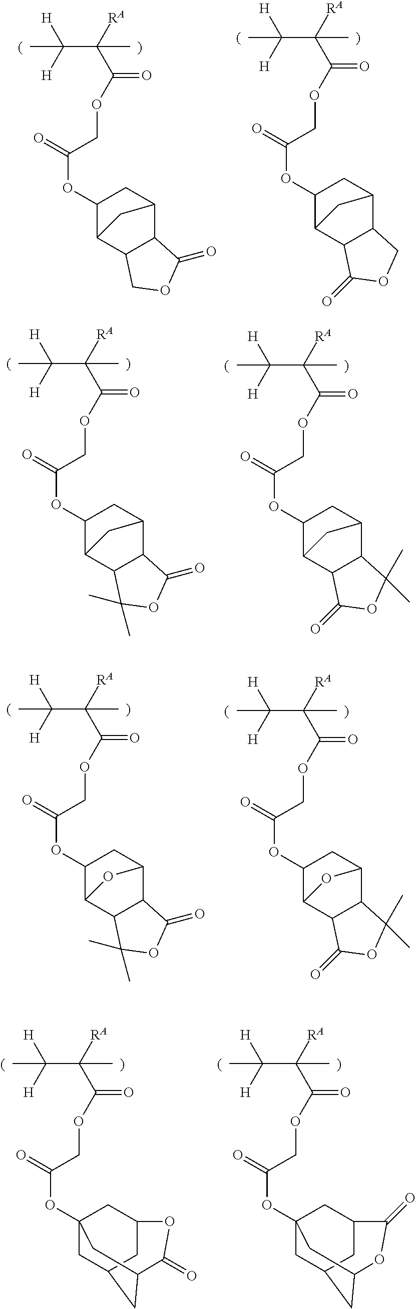

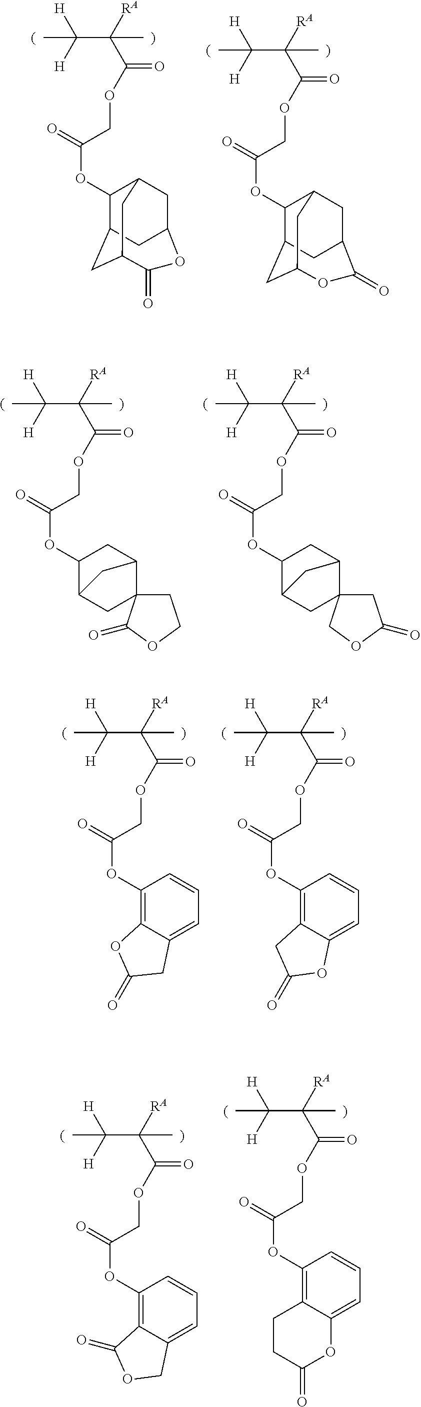

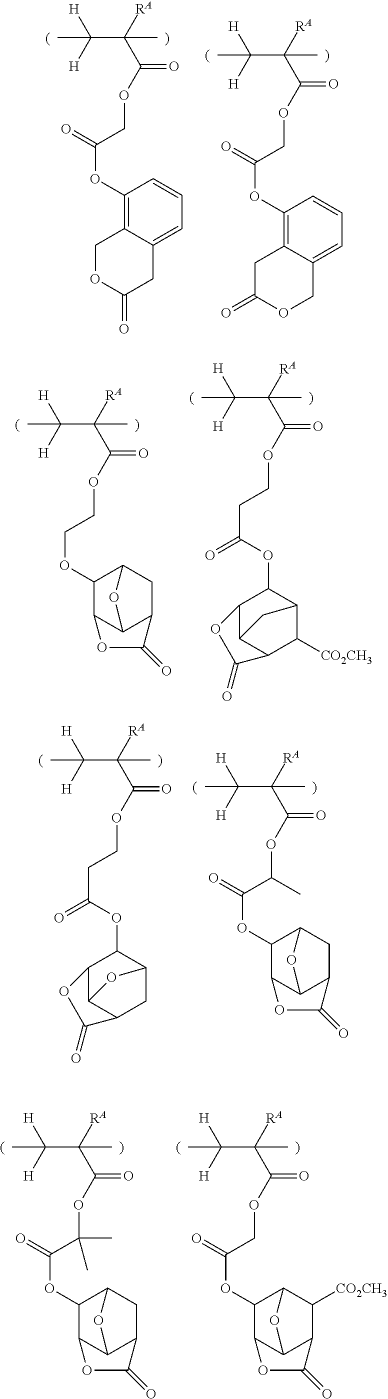

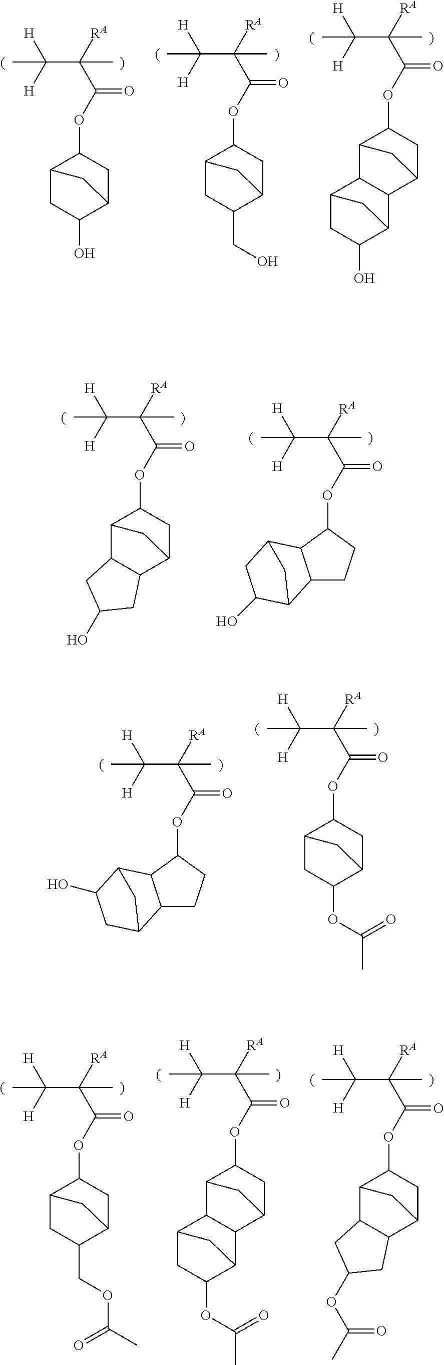

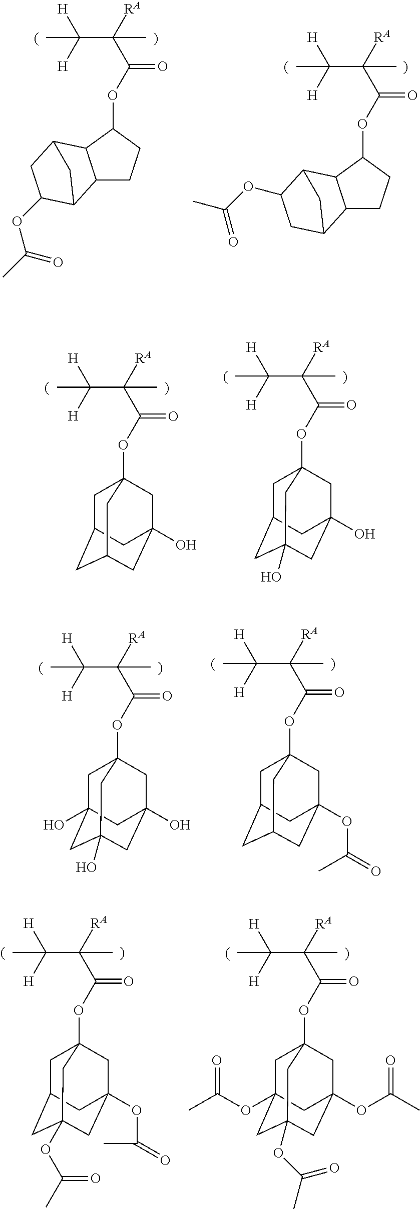

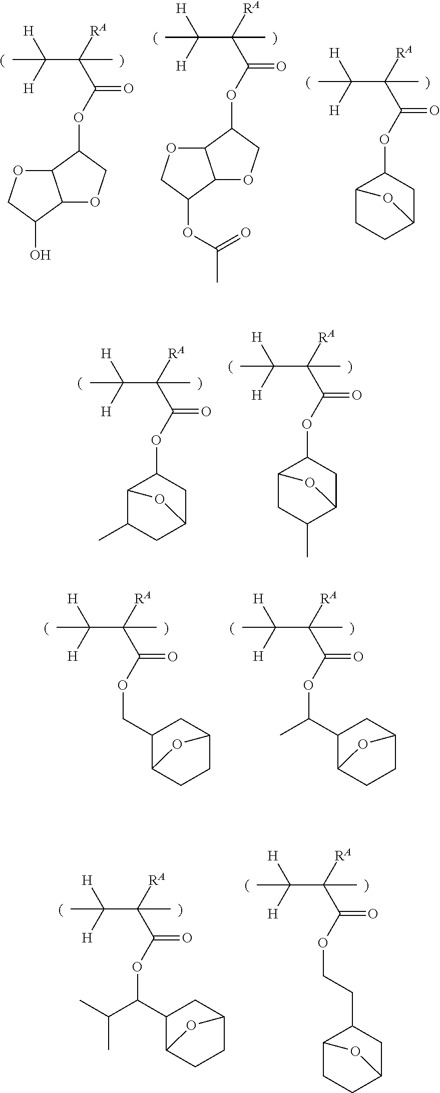

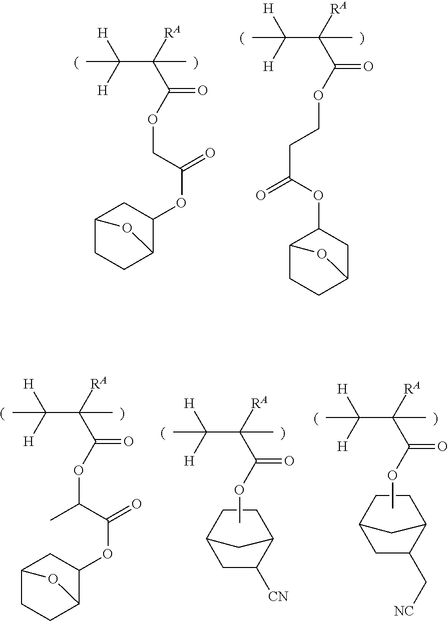

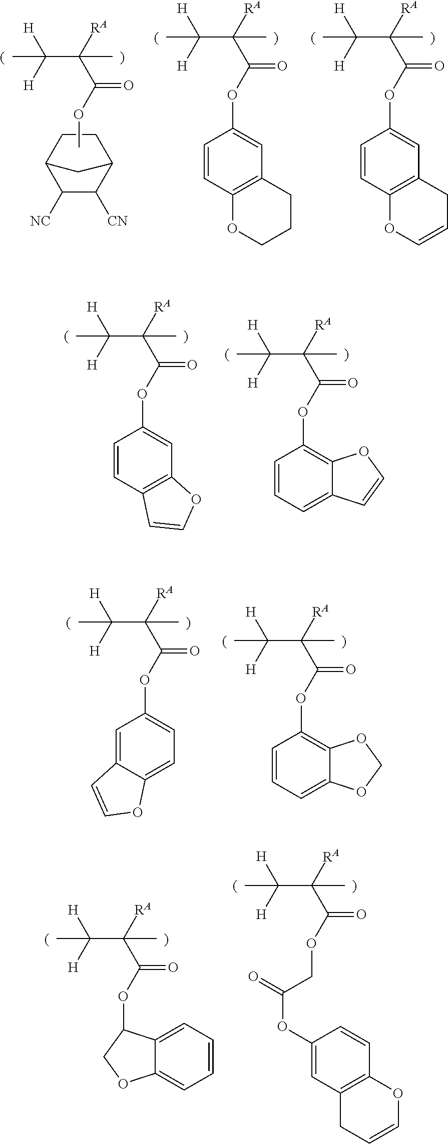

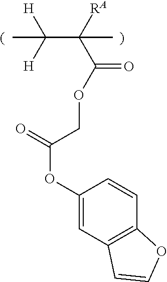

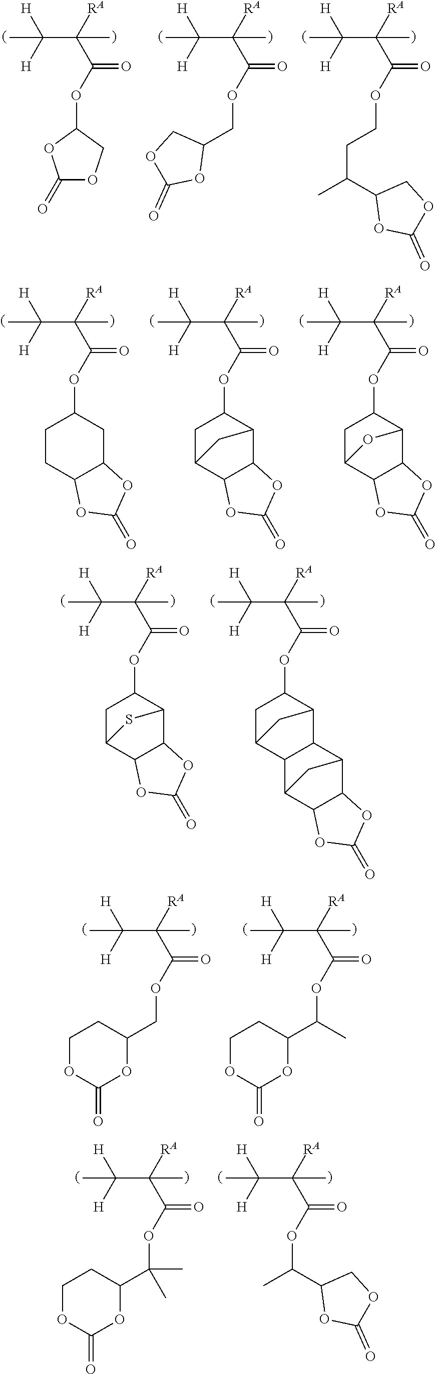

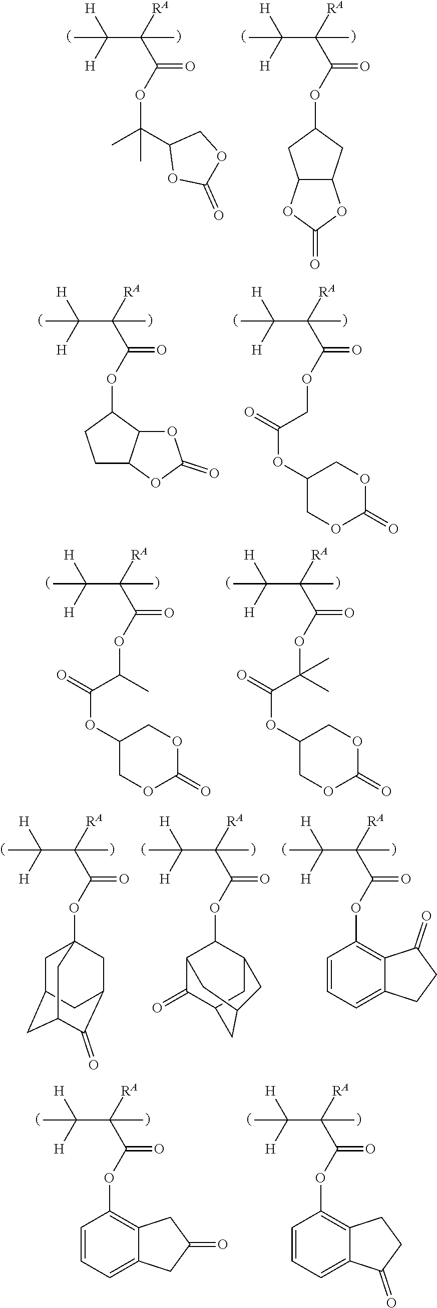

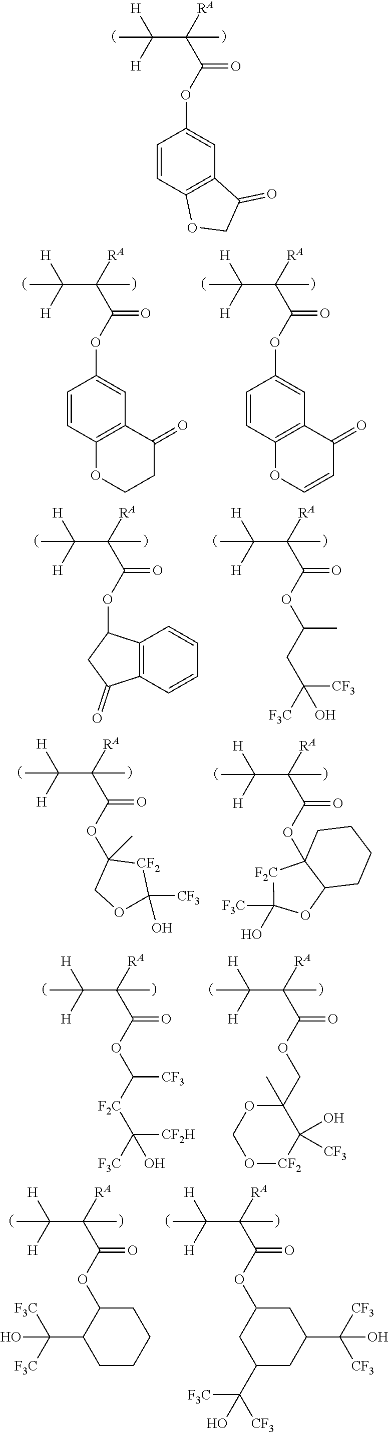

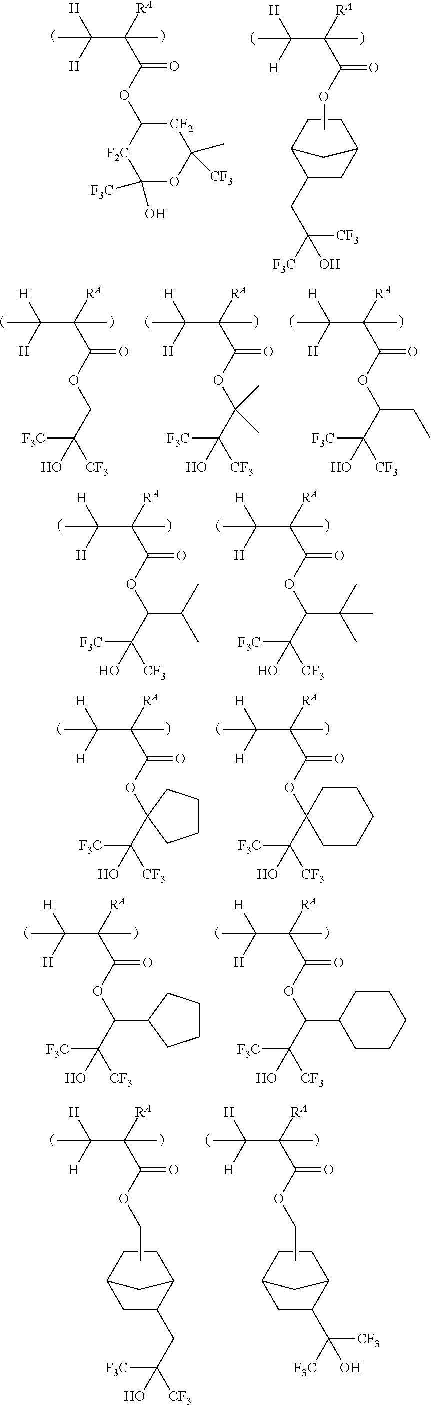

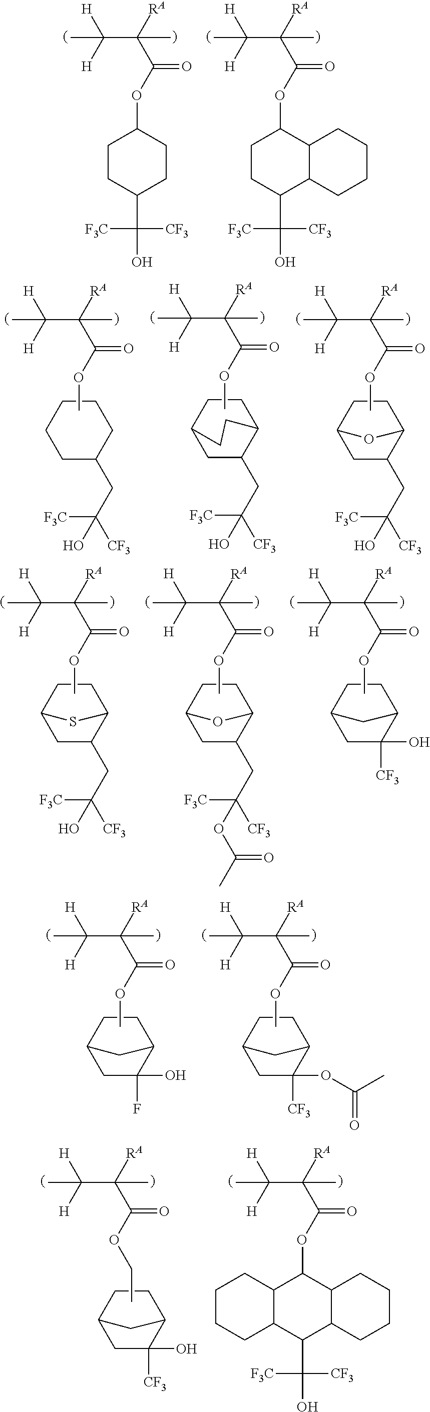

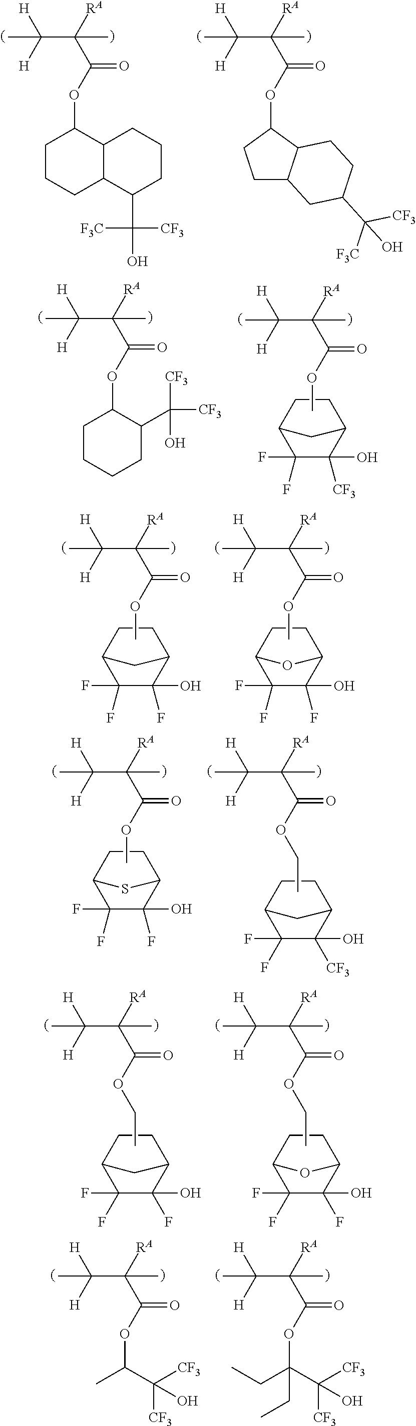

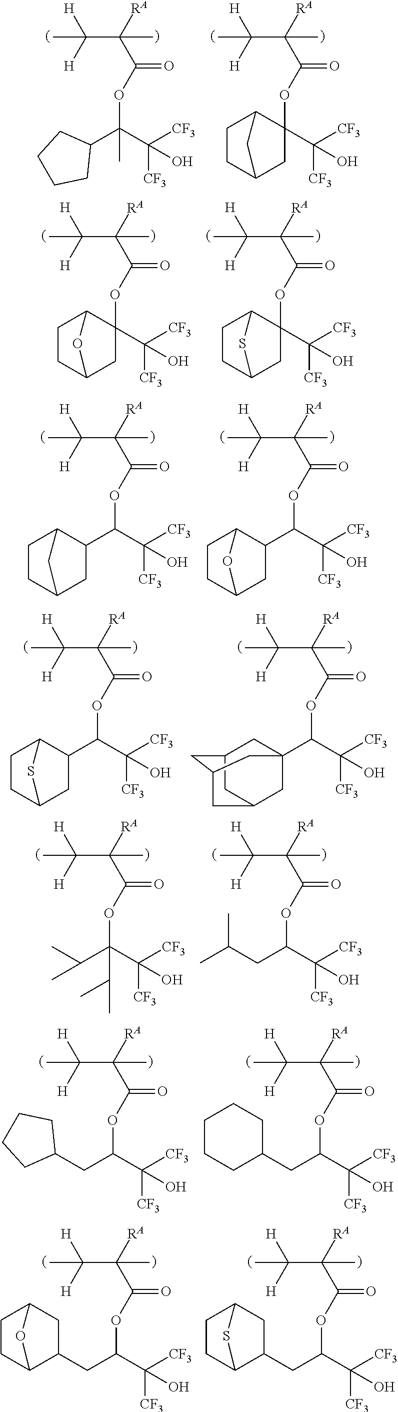

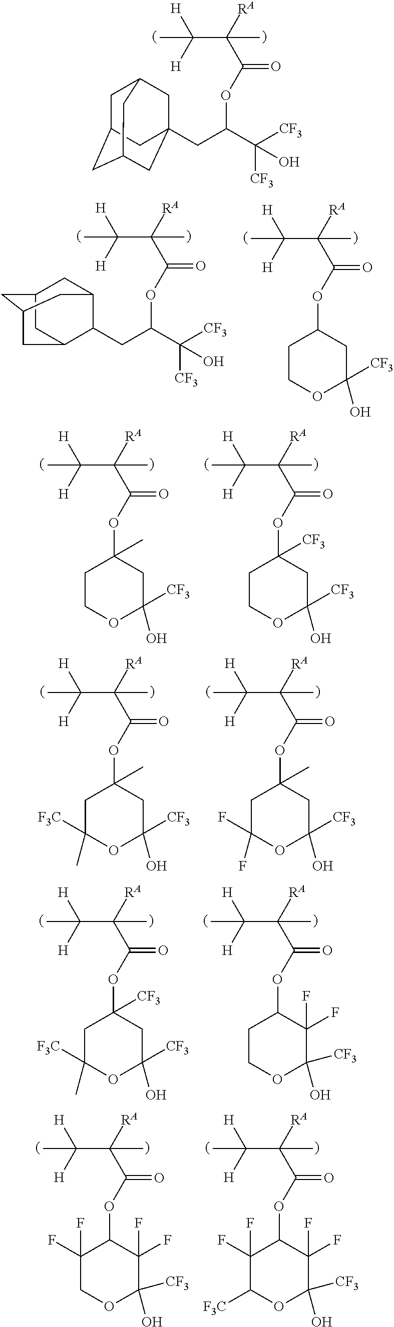

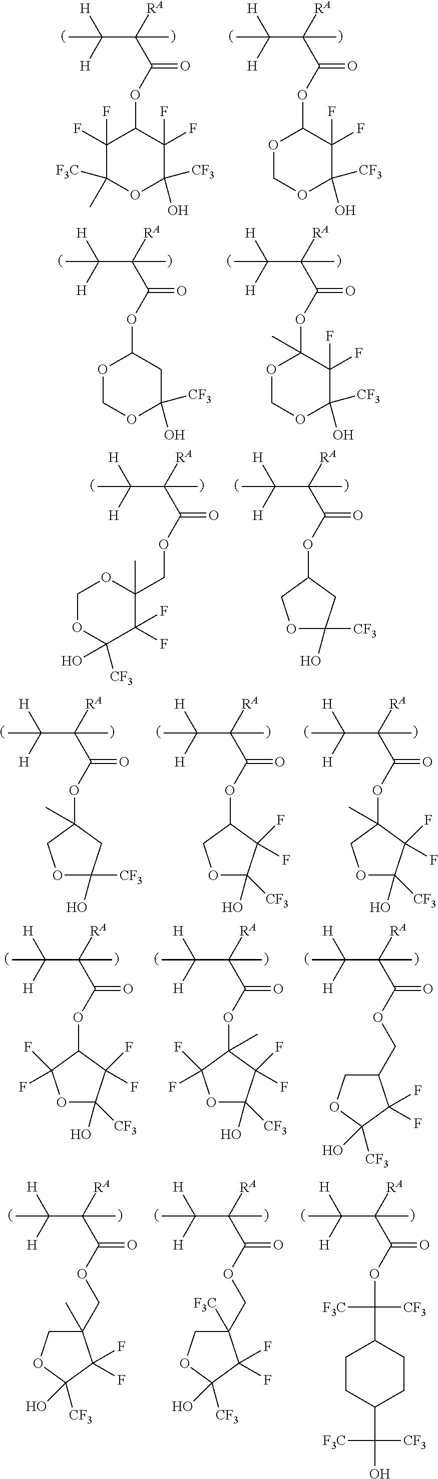

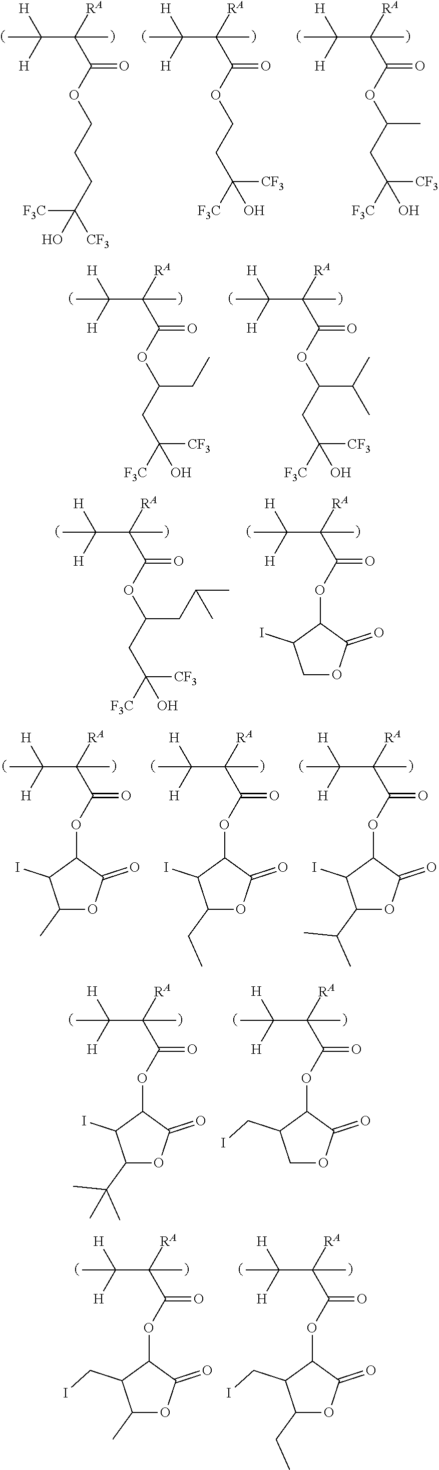

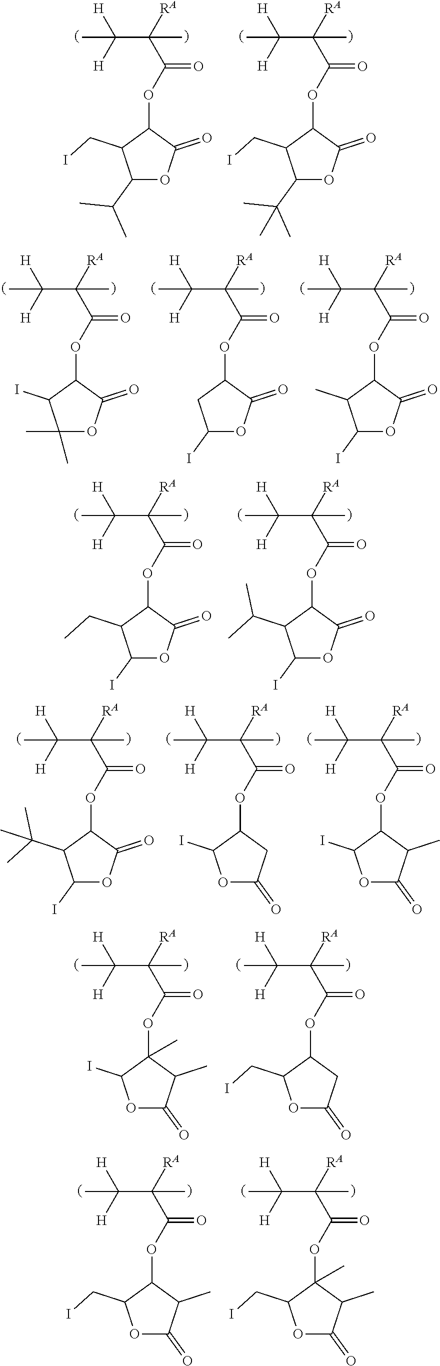

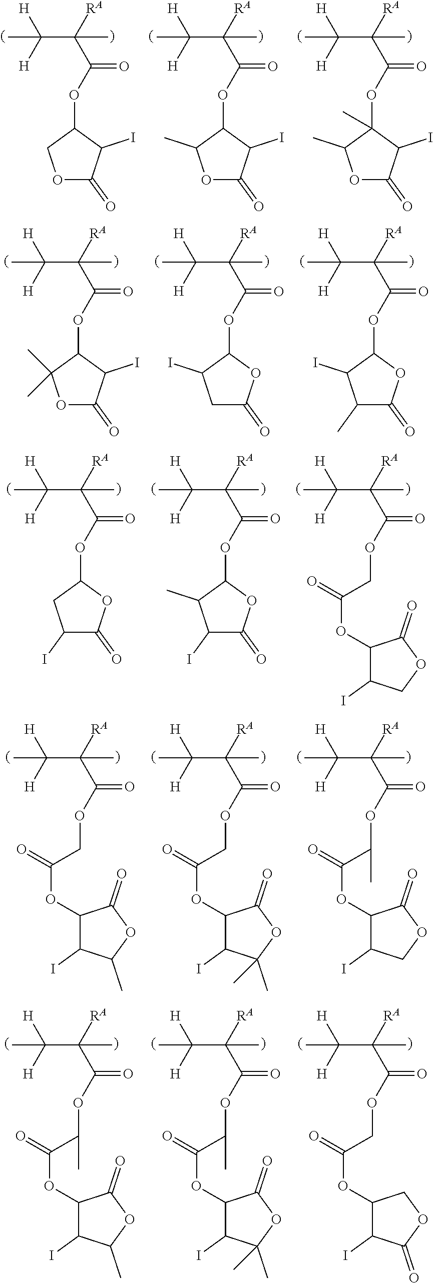

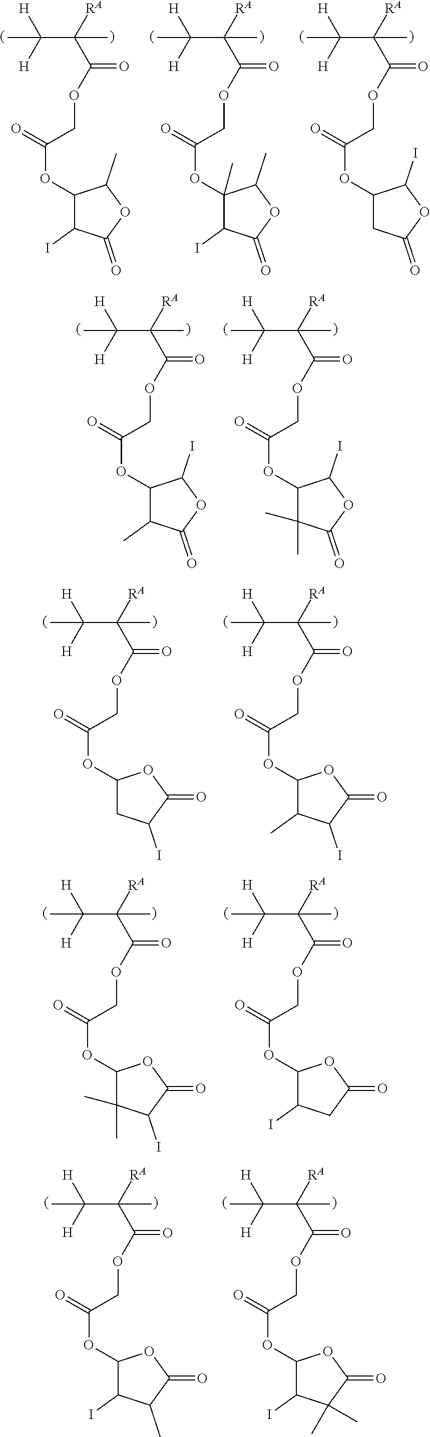

Specific examples of the repeating unit-b2 include, but are not limited to, the following. In the following formulae, R^{A} is as defined above, and Me represents a methyl group.

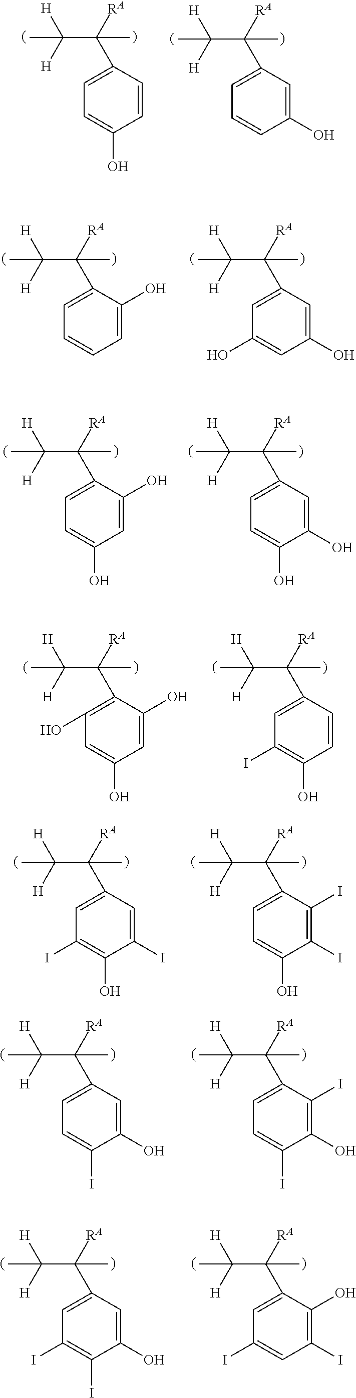

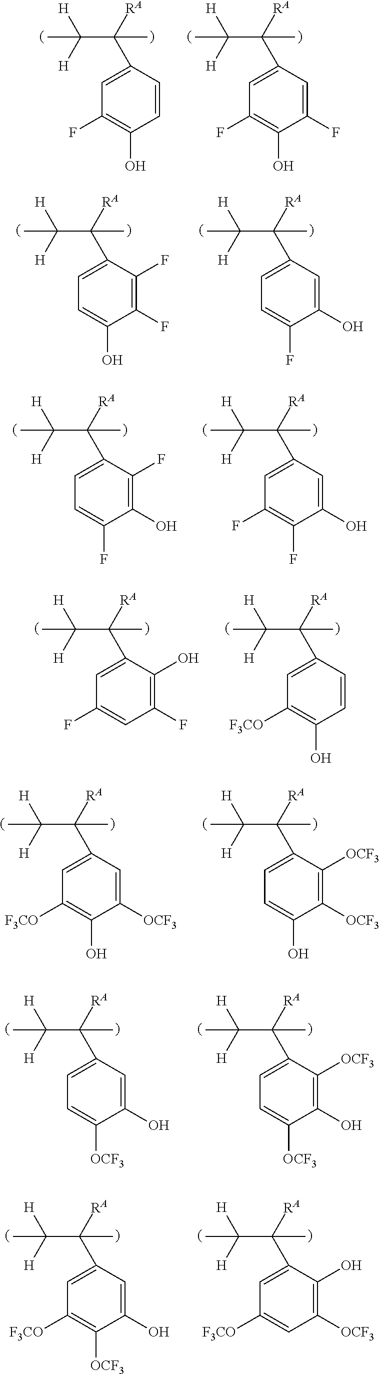

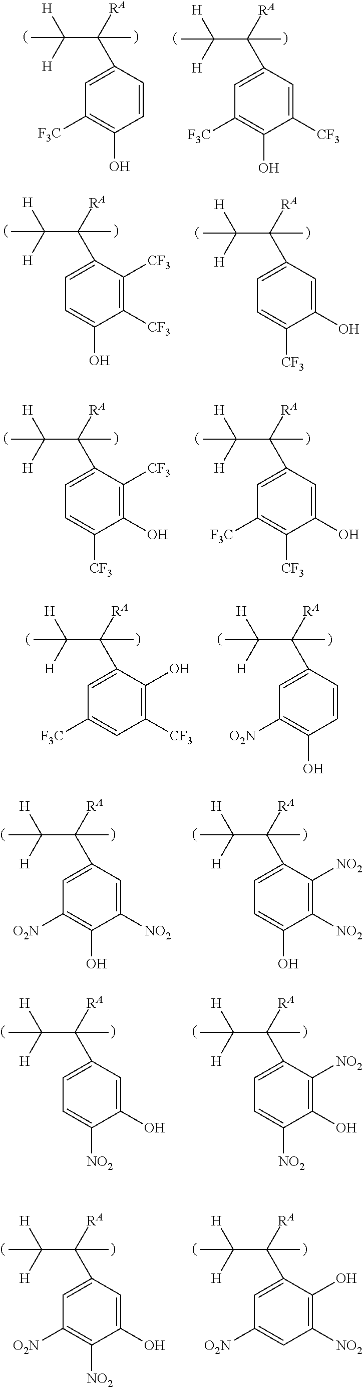

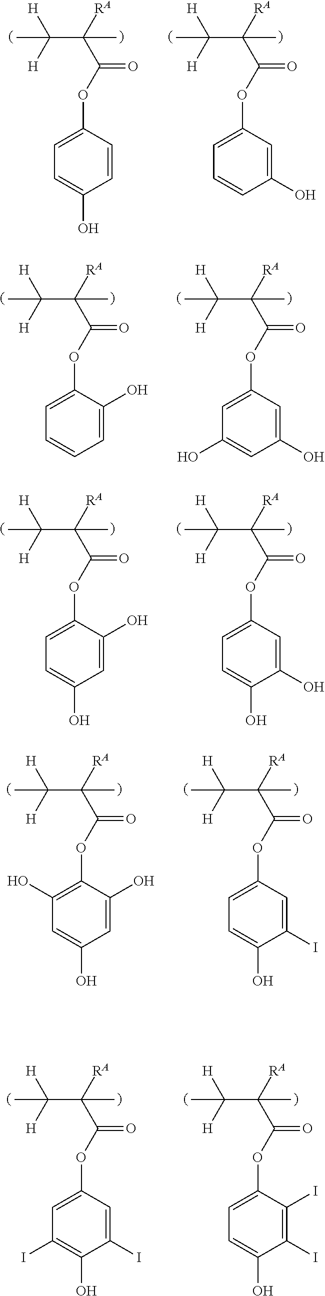

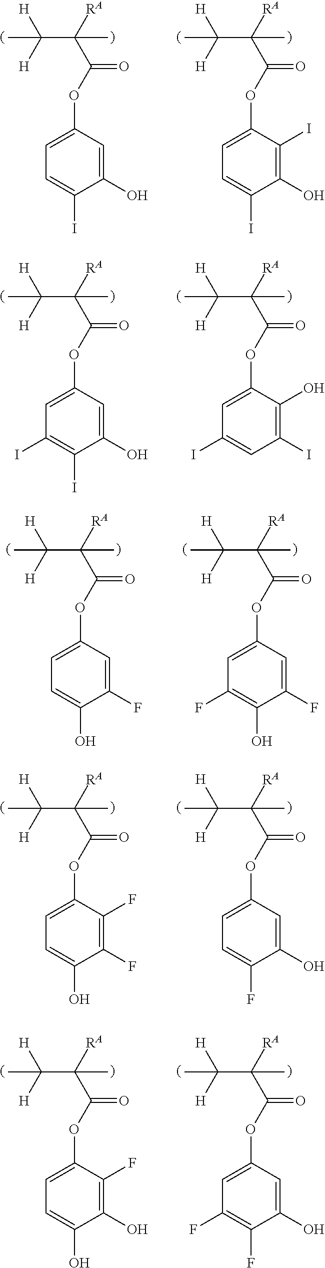

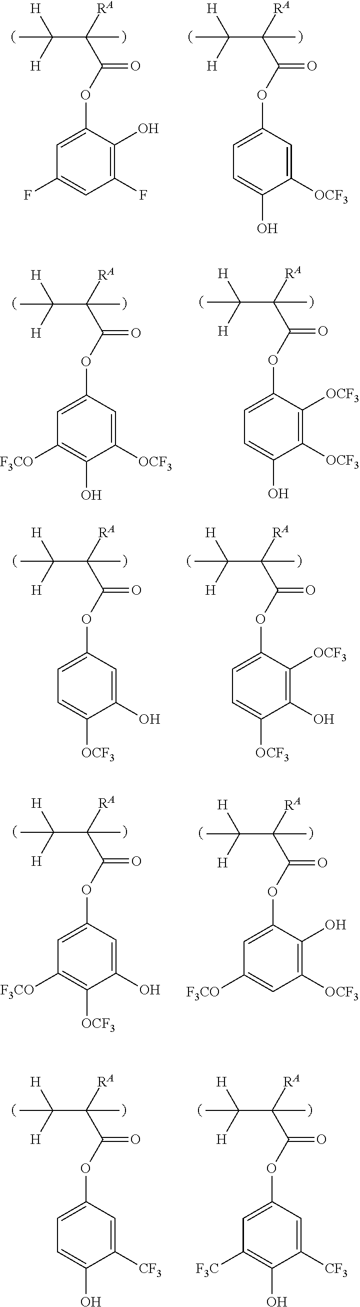

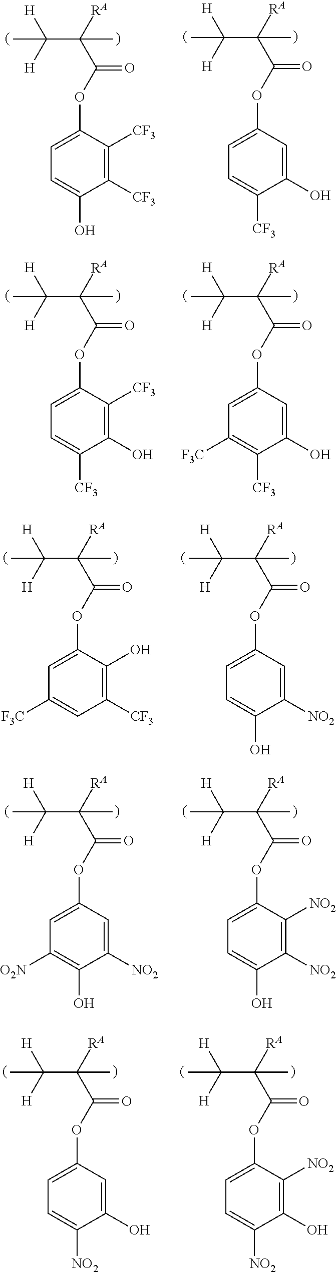

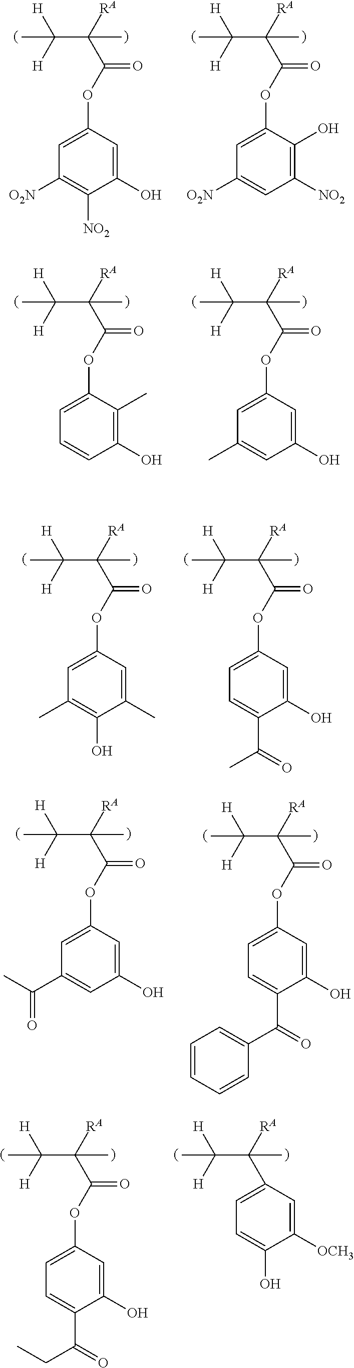

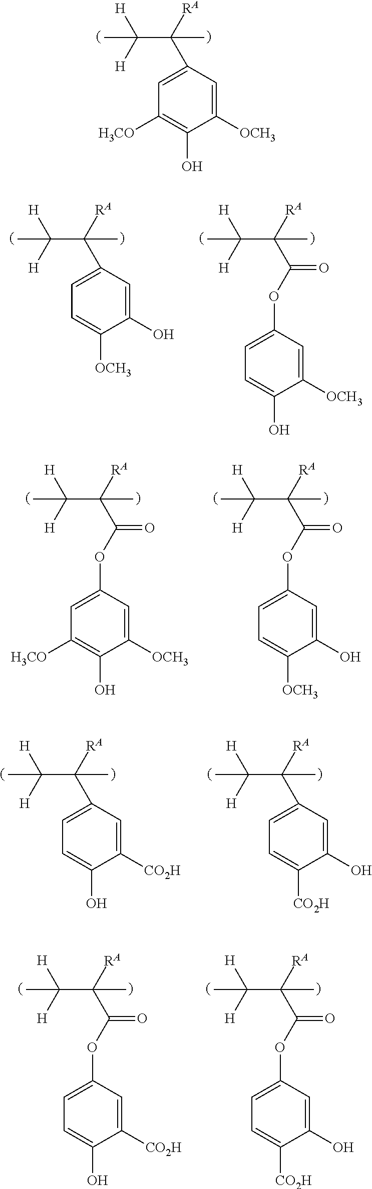

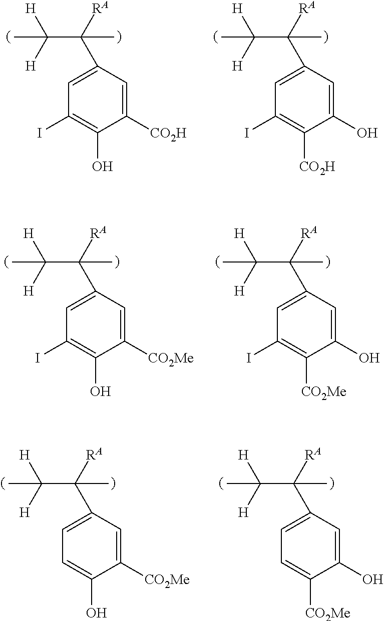

In ArF lithography, it is particularly preferable that the repeating unit-b1 or b2 has a lactone ring as a polar group, and in KrF lithography, EB lithography, and EUV lithography, it is preferable that the repeating unit has a phenol moiety.

The polymer may contain a repeating unit having a structure in which a hydroxy group is protected by an acid-labile group (hereinafter also referred to as “repeating unit-c”). The repeating unit-c is not particularly limited as long as it has one or more structures in which a hydroxy group is protected and the protecting group is decomposed by the action of an acid to generate a hydroxy group, but is preferably represented by the following general formula (c1).

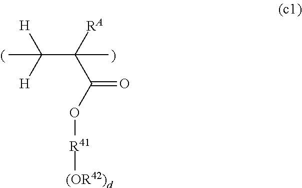

In the general formula (c1), R^{A} is as defined above. R⁴¹ represents a (d+1)-valent hydrocarbon group having 1 to 30 carbon atoms and optionally containing a heteroatom. R⁴² represents an acid-labile group. “d” represents an integer from 1 to 4.

In the general formula (c1), the acid-labile group represented by R⁴² may be any group that can be deprotected by the action of an acid to generate a hydroxy group. The structure of R⁴² is not particularly limited, but an acetal structure, a ketal structure, an alkoxycarbonyl group, or an alkoxymethyl group represented by the following general formula (c2) is preferable, and an alkoxymethyl group represented by the following general formula (c2) is particularly preferable.

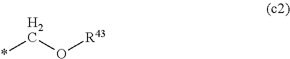

In the formula, “*” represents an attachment point; and R⁴³ represents a hydrocarbyl group having 1 to 15 carbon atoms.

Specific examples of the acid-labile group represented by R⁴², the alkoxymethyl group represented by the general formula (c2), and the repeating unit-c include the same ones as those given as examples in the explanation of the repeating unit-c described in JP2020-111564A.

The polymer may contain a repeating unit-d derived from indene, benzofuran, benzothiophene, acenaphthylene, chromone, coumarin, norbornadiene, or derivatives thereof. Specific examples of monomers that provide the repeating unit-d include, but are not limited to, the following.

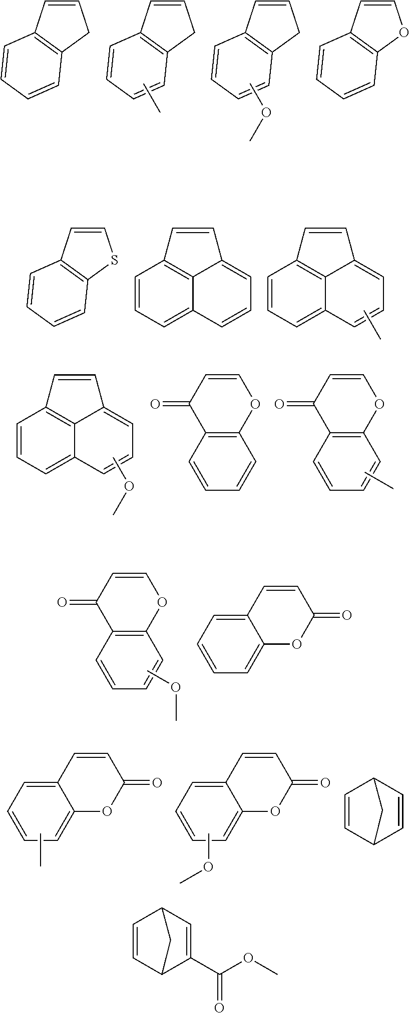

The polymer may further contain a repeating unit-e derived from indane, vinylpyridine, or vinylcarbazole.

In the polymer of the present invention, the content ratios of the repeating units-A, a1, a2, a3, b1, b2, c, d, and e are preferably 0<A≤0.4, 0≤a1≤0.8, 0≤a2≤0.8, 0≤a3≤0.6, 0<a1+a2+a3≤0.8, 0≤b1≤0.6, 0≤b2≤0.6, 0≤c≤0.5, 0≤d≤0.3, and 0≤e≤0.3, and more preferably 0<A≤0.3, 0≤a1≤0.7, 0≤a2≤0.7, 0≤a3≤0.5, 0<a1+a2+a3≤0.7, 0≤b1≤0.5, 0≤b2≤0.5, 0≤c≤0.3, 0≤d≤0.3, and 0≤e≤0.3. Note, 0<A+a1+a2+a3+b1+b2+c+d+e≤1.0.

The weight average molecular weight (Mw) of the polymer is preferably 1,000 to 500,000, and more preferably 3,000 to 100,000. When the Mw is within this range, sufficient dry etching resistance is obtained, and there is no risk of a decrease in resolution due to an inability to ensure a difference in dissolution rate before and after exposure. In the present invention, the Mw is a polystyrene-equivalent measurement value obtained by gel permeation chromatography (GPC) using THF or N,N-dimethylformamide (DMF) as a solvent.

Furthermore, since the influence of the Mw/Mn on the molecular weight distribution (Mw/Mn) of the polymer tends to become greater as the pattern rules become finer, in order to obtain a resist composition that is suitable for use with fine pattern dimensions, it is preferable that Mw/Mn is a narrow distribution of 1.0 to 2.0. Within the above range, there are few polymers with low or high molecular weights compared to Mw, and there is no risk of foreign matter being found on the pattern or the shape of the pattern deteriorating after exposure.

To synthesize the polymer, for example, a monomer that provides the repeating units described above may be polymerized by heating in an organic solvent together with a radical polymerization initiator.

Specific examples of organic solvents used during polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Specific examples of the polymerization initiator include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The amount of these initiators added is preferably 0.01 to 25 mol % based on the total amount of monomers to be polymerized. The reaction temperature is preferably 50 to 150° C., and more preferably 60 to 100° C. The reaction time is preferably 2 to 24 hours, and from the viewpoint of production efficiency, more preferably 2 to 12 hours.

The polymerization initiator may be added to the monomer solution and then fed into the reaction vessel together with it, or an initiator solution may be prepared separately from the monomer solution, and each of them may be fed independently into the reaction vessel. During the waiting time, radicals generated from the initiator may cause the polymerization reaction to proceed, resulting in the formation of ultra-high-molecular-weight molecules, and from the viewpoint of quality control, it is preferable to prepare and dropwise add the monomer solution and initiator solution separately. The acid-labile group may be used as is after being introduced into the monomer, and may be protected or partially protected after polymerization. In addition, a known chain transfer agent such as dodecyl mercaptan or 2-mercaptoethanol may be used in combination to adjust the molecular weight. In this case, the amount of these chain transfer agents added is preferably 0.01 to 20 mol % based on the total amount of monomers to be polymerized.

In the case of monomers containing hydroxy groups, the hydroxy groups may be substituted with acetal groups such as ethoxyethoxy groups, which are easily deprotected by acid, during polymerization, and then deprotected with weak acid and water after polymerization; alternatively, the hydroxy groups may be substituted with acetyl groups, formyl groups, pivaloyl groups, etc., and then alkaline hydrolysis may be performed after polymerization.

When copolymerizing hydroxystyrene or hydroxyvinylnaphthalene, the hydroxystyrene or hydroxyvinylnaphthalene may be polymerized by heating with other monomers in an organic solvent after adding a radical polymerization initiator, or it is possible to use acetoxystyrene or acetoxyvinylnaphthalene, and after polymerization, deprotect the acetoxy group by alkaline hydrolysis to produce polyhydroxystyrene or hydroxypolyvinylnaphthalene.

As the base for alkaline hydrolysis, ammonia water, triethylamine, etc. can be used. The reaction temperature is preferably −20 to 100° C., and more preferably 0 to 60° C. The reaction time is preferably 0.2 to 100 hours, and more preferably 0.5 to 20 hours.

The amount of each monomer in the monomer solution may be appropriately set so as to obtain the preferred content ratios of the repeating units described above.

Regarding the polymer obtained by the above-mentioned manufacturing method, the reaction solution obtained by the polymerization reaction may be handled as the final product, or the powder obtained through a purification process such as a reprecipitation method in which the reaction solution is added to a poor solvent to obtain a powder may be handled as the final product; however, from the viewpoint of work efficiency and quality stabilization, it is preferable to handle as the final product the polymer solution obtained by dissolving the powder obtained through the purification process in a solvent.

Specific examples of the solvent to be used in this case include as described in paragraphs [0144] to [0145] of JP2008-111103A; ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono tert-butyl ether acetate; lactones such as GBL; alcohols such as diacetone alcohol (DAA); high-boiling-point alcohol solvents such as diethylene glycol, propylene glycol, glycerin, 1,4-butanediol, and 1,3-butanediol; and mixed solvents of these.

In the polymer solution, the concentration of the polymer is preferably 0.01 to 30% by mass, and more preferably 0.1 to 20% by mass.

The reaction solution and the polymer solution are preferably filtered. Filtering can remove foreign matter and gels that may cause defects, and is effective for stabilizing quality.

The material of the filter used for the above-mentioned filtration may be a fluorocarbon-based, cellulose-based, nylon-based, polyester-based, or hydrocarbon-based material, but in the filtration step of the resist composition, a filter made of a fluorocarbon-based material known as Teflon (registered trademark), a hydrocarbon-based material such as polyethylene or polypropylene, or nylon is preferable. The pore size of the filter can be appropriately selected according to the desired degree of cleanliness, but is preferably 100 nm or less, and more preferably 20 nm or less. These filters may be used alone or in a combination of a plurality of them. The filtration method may involve passing the solution through the filter only once, but it is more preferable to circulate the solution and filter it a plurality of times. The filtration step can be performed in any order and any number of times in the polymer manufacturing process, but it is preferable to filter the reaction solution after the polymerization reaction, the polymer solution, or both.

### [Chemically Amplified Resist Composition]

The present invention provides a chemically amplified resist composition containing a base polymer that contains the polymer (A) described above.

### [Base Polymer (A)]

The chemically amplified resist composition of the present invention contains, as component (A), a base polymer containing the above-mentioned polymer.

The polymer may be used alone or in a combination of two or more types having different composition ratios, Mw, and/or Mw/Mn. In addition to the above-mentioned polymers, the base polymer (A) may contain a hydrogenated ring-opening metathesis polymer, and the polymers described in JP2003-066612A can be used for that.

In this case, it is preferable that the chemically amplified resist composition further contains one or more selected from an organic solvent (B), a quencher (C), a photo-acid generator (D) other than the monomeric photo-acid generator, and a surfactant (E). Each component is explained in detail below.

### [Organic Solvent (B)]

The chemically amplified resist composition of the present invention may contain an organic solvent as component (B). The organic solvent (B) is not particularly limited as long as it can dissolve each of the components described above and below. Specific examples of such organic solvents include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; ketoalcohols such as DAA; ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono tert-butyl ether acetate; lactones such as GBL; and mixed solvents of these.

Among these organic solvents, 1-ethoxy-2-propanol, PGMEA, cyclohexanone, GBL, ethyl lactate, DAA, and mixed solvents of these are preferable, as they have particularly excellent solubility for the base polymer for component (A).

In the chemically amplified resist composition of the present invention, the content of the organic solvent (B) is preferably 200 to 7,000 parts by mass, and more preferably 400 to 5,000 parts by mass, per 80 parts by mass of the base polymer (A). The organic solvent (B) may be used alone or in a combination of two or more types.

### [Quencher (C)]

The chemically amplified resist composition of the present invention may contain a quencher as component (C). In the present invention, a quencher is a material that traps the acid generated by the photo-acid generator in the chemically amplified resist composition, thereby preventing the acid from diffusing into unexposed areas, so that the desired pattern can be formed.

Specific examples of the quencher (C) include onium salts represented by the following general formula (1) or (2).

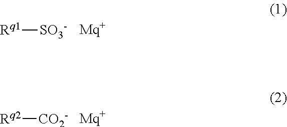

In the general formula (1), R^{q1} represents a hydrogen atom, or a hydrocarbyl group having 1 to 40 carbon atoms and optionally containing a heteroatom, except for those in which the hydrogen atom bonded to the carbon atom at the α-position of the sulfo group is substituted with a fluorine atom or a fluoroalkyl group. In the general formula (2), R^{q2} represents a hydrogen atom, or a hydrocarbyl group having 1 to 40 carbon atoms and optionally containing a heteroatom.

Specific examples of hydrocarbyl groups having 1 to 40 carbon atoms represented by R^{q1} include alkyl groups having 1 to 40 carbon atoms, such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, and n-decyl groups; cyclic saturated hydrocarbyl groups having 3 to 40 carbon atoms, such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decyl, and adamantyl groups; and aryl groups with 6 to 40 carbon atoms, such as phenyl, naphthyl, and anthracenyl groups. Furthermore, some or all of the hydrogen atoms of the hydrocarbyl groups may be substituted with a group containing a heteroatom such as an oxygen atom, a sulfur atom, a nitrogen atom, or a halogen atom, and some of the —CH₂— of the hydrocarbyl groups may be substituted with a group containing a heteroatom such as an oxygen atom, a sulfur atom, or a nitrogen atom, and as a result, they may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group.

Specific examples of hydrocarbyl groups represented by R^{q2} include, in addition to the substituents given as specific examples of R^{q1}, fluorinated saturated hydrocarbyl groups such as a trifluoromethyl group and a trifluoroethyl group, and fluorinated aryl groups such as a pentafluorophenyl group and a 4-trifluoromethylphenyl group.

Specific examples of the anion of the onium salt represented by the general formula (1) include, but are not limited to, the following.

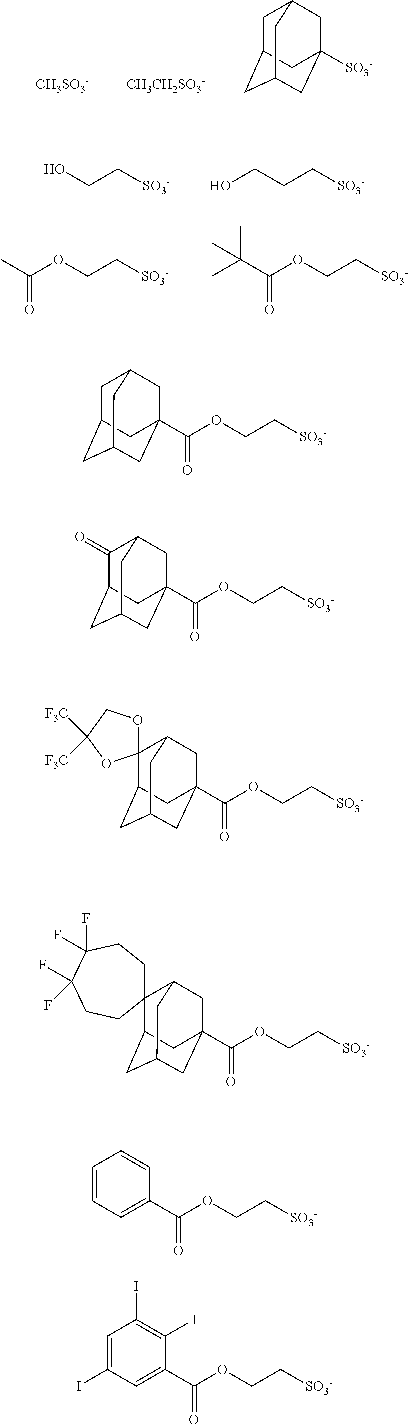

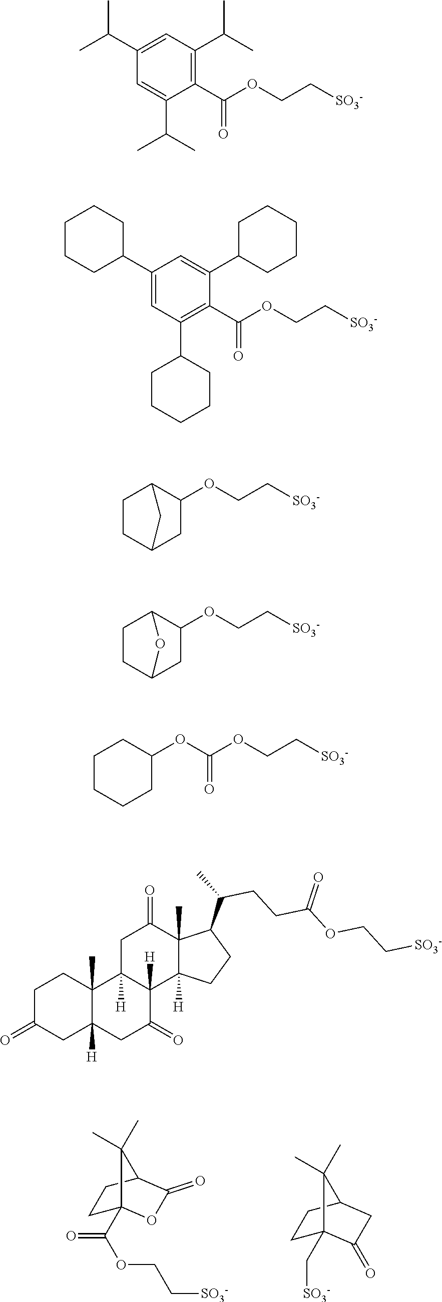

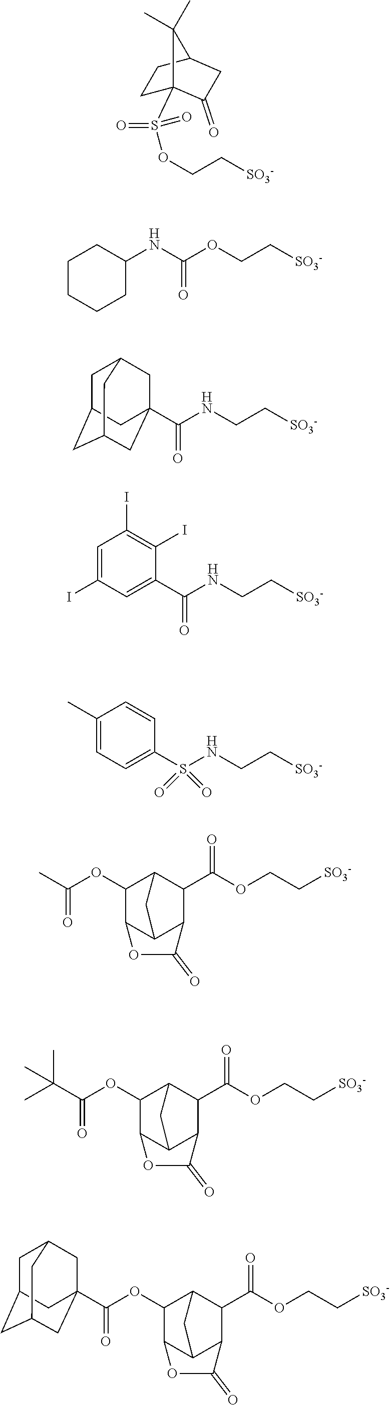

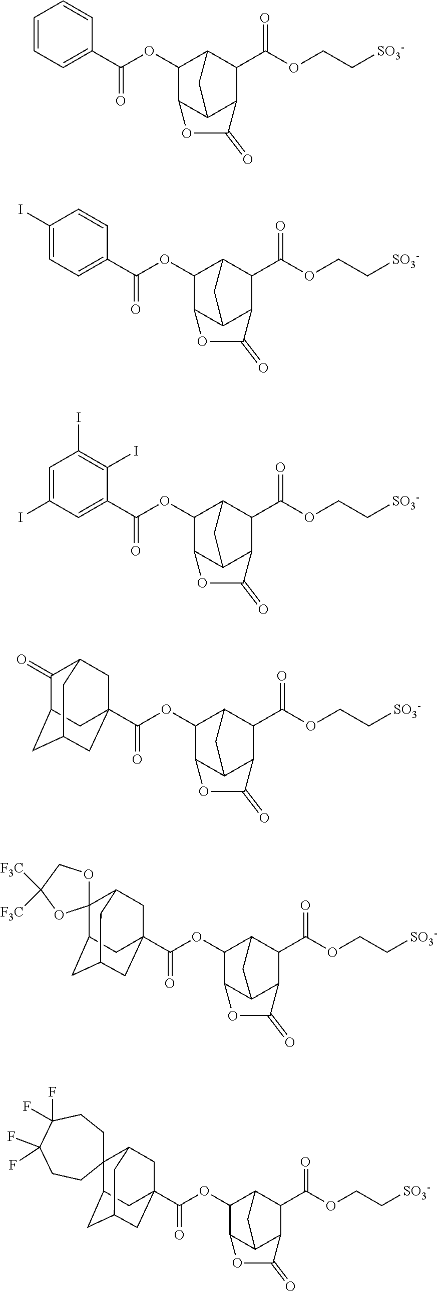

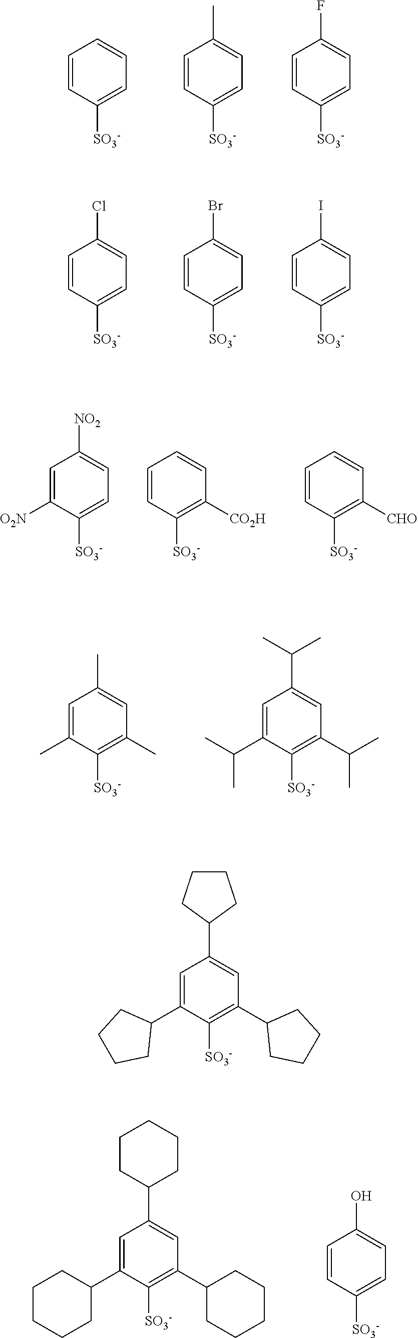

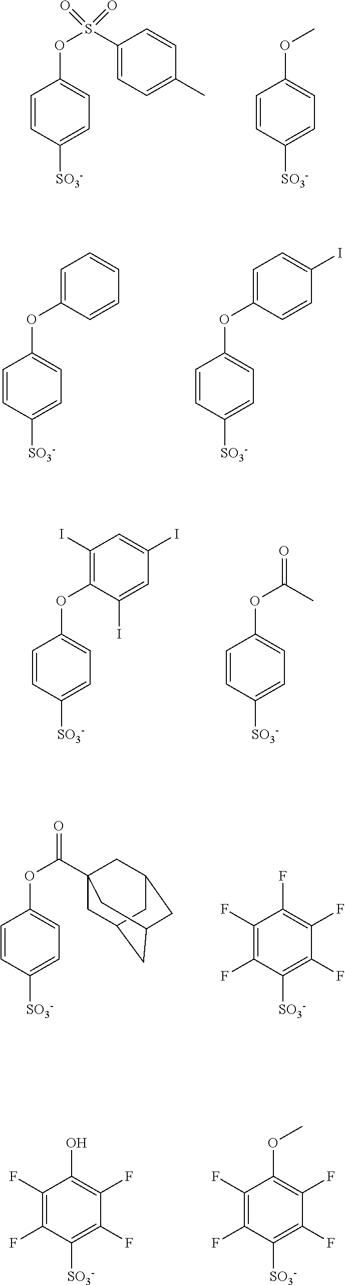

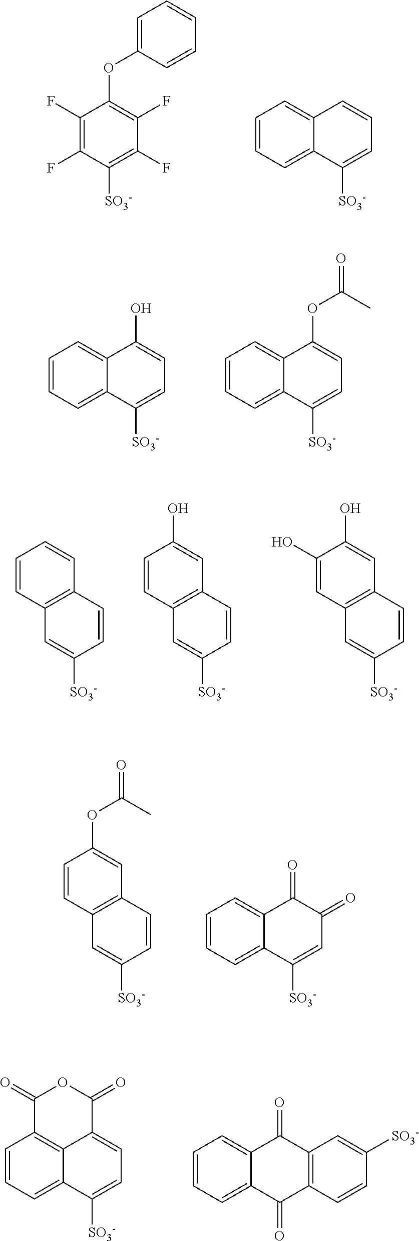

Specific examples of the anion of the onium salt represented by the general formula (2) include, but are not limited to, the following.

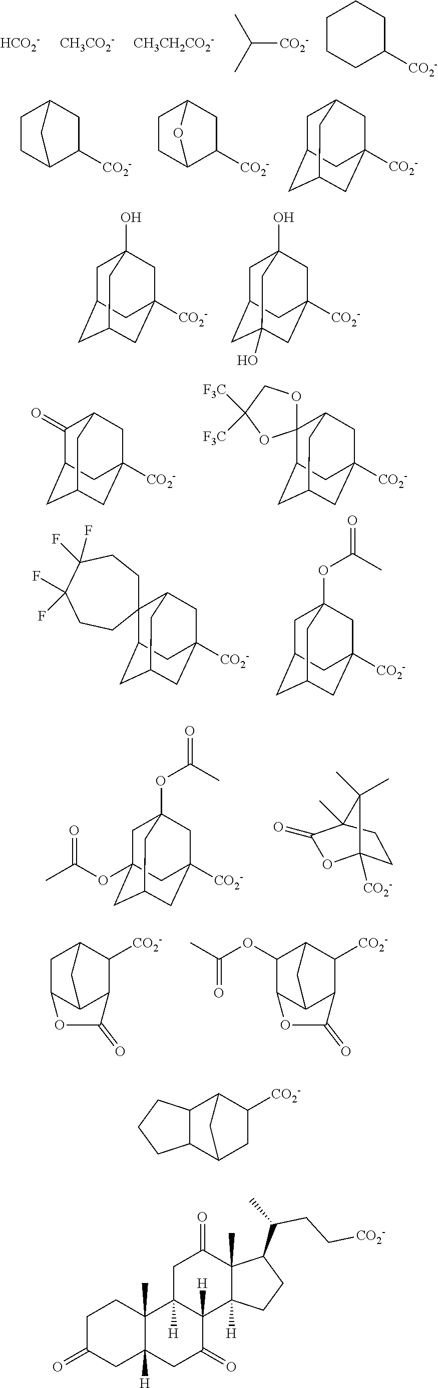

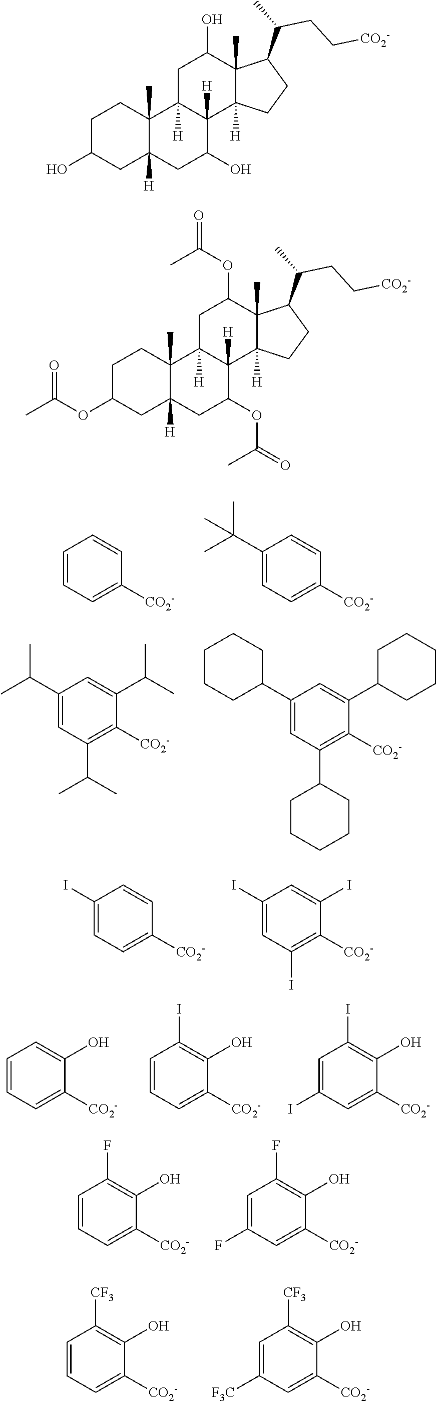

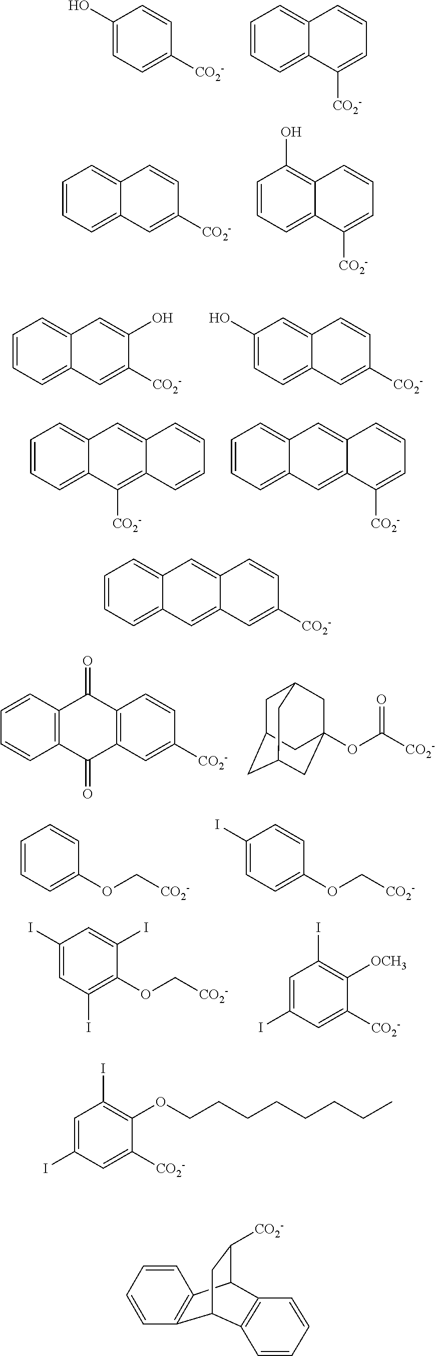

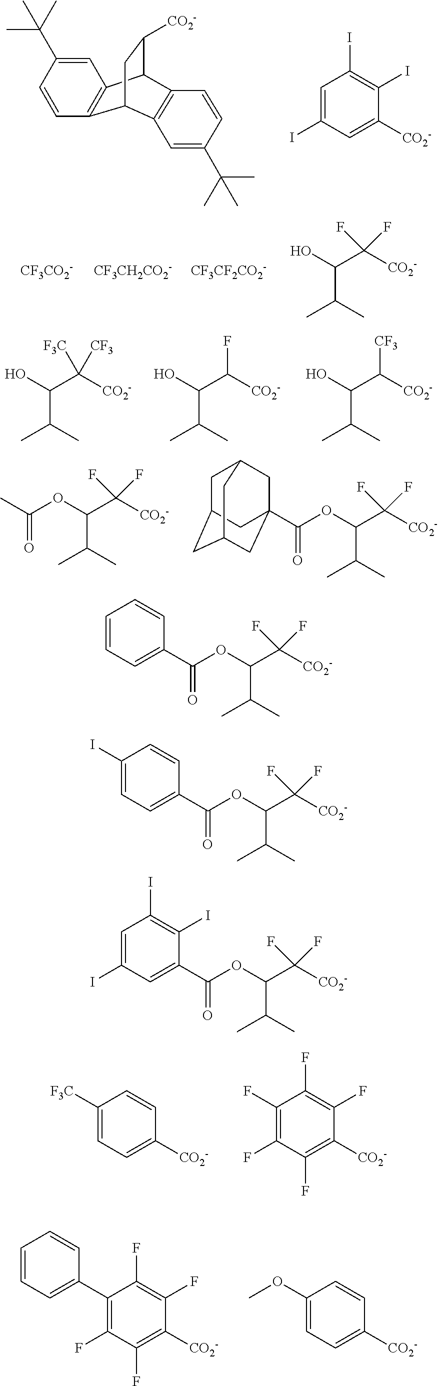

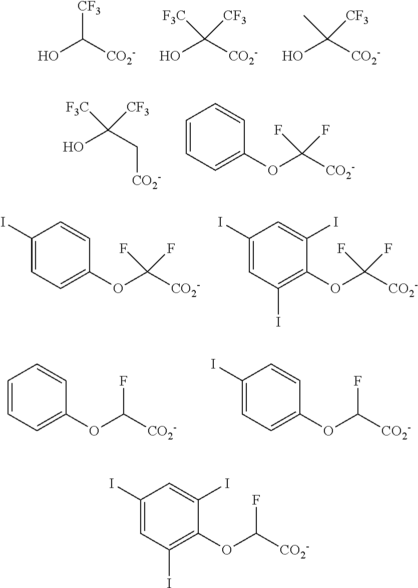

In the general formulae (1) and (2), Mq⁺ is an onium cation. Examples of the onium cation include a sulfonium cation, an iodonium cation, and an ammonium cation. Specific examples of the sulfonium cation include those given as specific examples of the sulfonium cation in the general formula (A), but are not limited thereto.

Specific examples of the iodonium cation include those given as specific examples of the iodonium cation in the general formula (A), but are not limited thereto.

Specific examples of the ammonium cation include those represented by the following general formula (am-1).

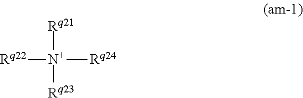

In the general formula (am-1), R^{q21} to R^{q24} each independently represent a hydrocarbyl group having 1 to 40 carbon atoms and optionally containing a heteroatom. Furthermore, R^{q21} and R^{q22} may be bonded to each other to form a ring together with the nitrogen atom to which they are bonded. Specific examples of the hydrocarbyl group include the same ones as those given as examples of the hydrocarbyl group represented by R¹ in the explanation of the general formula (A).

Specific examples of the ammonium cation represented by the general formula (am-1) include, but are not limited to, the following.

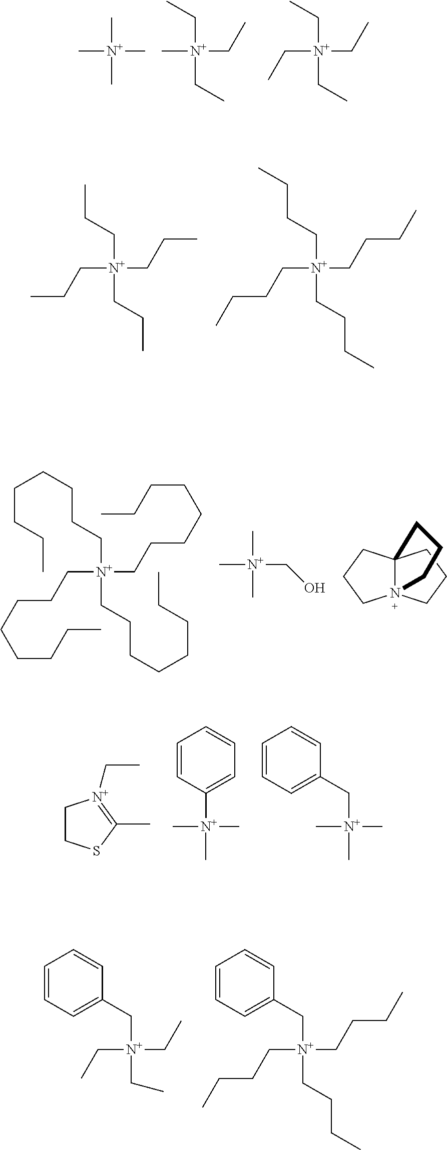

Specific examples of the onium salt represented by the general formula (1) or (2) include any combination of the above-mentioned anions and cations. These onium salts can be easily prepared by ion exchange reactions using known organic chemistry methods. For the ion exchange reactions, for example, JP2007-145797A can be referred to.

The onium salt represented by the general formula (1) or (2) acts as a quencher in the chemically amplified resist composition of the present invention. This is because each counter anion of the onium salt is the conjugate base of a weak acid. The term “weak acid” used herein means an acidity that is not capable of deprotecting the acid-labile group of the acid-labile-group-containing unit used in the base polymer. The onium salt represented by the general formula (1) or (2) functions as a quencher when used in combination with an onium salt type photo-acid generator having, as a counter anion, a conjugate base of a strong acid such as a sulfonic acid whose α-position is fluorinated. That is, when an onium salt that generates a strong acid such as a sulfonic acid whose α-position is fluorinated is mixed with an onium salt that generates a weak acid such as a non-fluorinated sulfonic acid or carboxylic acid, if the strong acid generated from the photo-acid generator by irradiation with a high-energy beam collides with an onium salt having an unreacted weak acid anion, the weak acid is released by salt exchange and an onium salt having a strong acid anion is generated. In this process, the strong acid is exchanged for a weaker acid with lower catalytic activity, and the acid becomes deactivated, enabling control of acid diffusion.

Furthermore, as the quencher (C), onium salts having a sulfonium cation and a phenoxide anion moiety in the same molecule as described in JP6848776B2, onium salts having a sulfonium cation and a carboxylate anion moiety in the same molecule as described in JP6583136B2 and JP2020-200311A, and onium salts having an iodonium cation and a carboxylate anion moiety in the same molecule as described in JP6274755B2 can also be used.

Here, when the photo-acid generator that generates a strong acid is an onium salt, the strong acid generated by irradiation with a high-energy beam can be exchanged for a weak acid as described above, but on the other hand, it is thought that the weak acid generated by irradiation with a high-energy beam collides with the unreacted onium salt that generates strong acid, making it difficult to carry out salt exchange. This is due to the phenomenon that the onium cation tends to form an ion pair with the anion of a stronger acid.

When the chemically amplified resist composition of the present invention contains an onium salt represented by general formula (1) or (2) as the quencher (C), the content thereof is preferably 0.1 to 20 parts by mass, and more preferably 0.1 to 10 parts by mass, per 80 parts by mass of the base polymer (A). When the content of the onium salt type quencher for component (C) is within the above range, the resolution is good and there is no significant decrease in sensitivity, so it is preferable. The onium salts represented by the general formula (1) or (2) may be used alone or in a combination of two or more types.

The chemically amplified resist composition of the present invention may contain a nitrogen-containing compound as a quencher (C). Specific examples of the nitrogen-containing compound for component (C) include primary, secondary, or tertiary amine compounds described in paragraphs [0146] to [0164] of JP2008-111103A, in particular, amine compounds having a hydroxy group, an ether bond, an ester bond, a lactone ring, a cyano group, or a sulfonate ester bond. Further examples include compounds in which a primary or secondary amine is protected with a carbamate group, as described in JP3790649B2.

Furthermore, a sulfonic acid sulfonium salt having a nitrogen-containing substituent may be used as the nitrogen-containing compound. Such compounds function as quenchers in unexposed areas, and lose their quencher ability by neutralizing with the acid generated by themselves in exposed areas, functioning as so-called photodegradable bases. By using a photodegradable base, the contrast between exposed and unexposed areas can be further enhanced. For photodegradable bases, reference can be made to, for example, JP2009-109595A and JP2012-046501A.

When the chemically amplified resist composition of the present invention contains a nitrogen-containing compound as a quencher (C), the content thereof is preferably 0.001 to 12 parts by mass, and more preferably 0.01 to 8 parts by mass, per 80 parts by mass of the base polymer (A). The nitrogen-containing compounds may be used alone or in a combination of two or more types.

### [Other Photo-Acid Generators (D)]

The chemically amplified resist composition of the present invention may contain, as component (D), a photo-acid generator other than the monomeric photo-acid generator (hereinafter also referred to as “other photo-acid generators”). The other photo-acid generators are not particularly limited as long as they are a compound that generates an acid when irradiated with a high-energy beam. Suitable other photo-acid generators (D) include those represented by the following general formula (3) or (4).

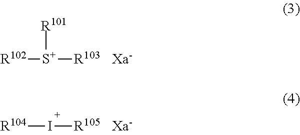

In the general formula (3), R¹⁰¹ to R¹⁰⁵ each independently represent a halogen atom, or a hydrocarbyl group having 1 to 20 carbon atoms and optionally containing a heteroatom. Furthermore, any two of R¹⁰¹, R¹⁰², and R¹⁰³ may be bonded to each other to form a ring together with the sulfur atom to which they are bonded.

Specific examples of the sulfonium cation of the sulfonium salt represented by the general formula (3) include those given as specific examples of the sulfonium cation in the general formula (A), but are not limited thereto. Specific examples of the iodonium cation of the iodonium salt represented by the general formula (4) include those given as specific examples of the iodonium cation in the general formula (A), but are not limited thereto.

In the general formulae (3) and (4), Xa⁻ represents the anion of a strong acid. Examples of the anion of the strong acid include those represented by any of the following general formulae (Xa-1) to (Xa-4).

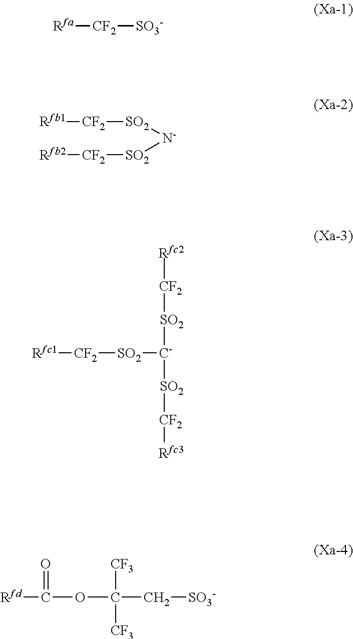

In the general formula (Xa-1), R^{fa} represents a fluorine atom, or a hydrocarbyl group having 1 to 40 carbon atoms and optionally containing a heteroatom. The hydrocarbyl group may be saturated or unsaturated, and may be linear, branched, or cyclic. Specific examples thereof include the same ones as those given as example of the hydrocarbyl group represented by R^{fa1} in the general formula (Xa-1-1) described later.

The anion represented by the general formula (Xa-1) is preferably one represented by the following general formula (Xa-1-1).

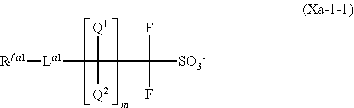

In the general formula (Xa-1-1), Q¹ and Q² each independently represent a hydrogen atom, a fluorine atom, or a fluorinated saturated hydrocarbyl group having 1 to 6 carbon atoms, but in order to improve solvent solubility, it is preferable that at least one of them is a trifluoromethyl group. “m” represents an integer from 0 to 4, and is particularly preferably 1. R^{fa1} represents a hydrocarbyl group having 1 to 35 carbon atoms and optionally containing a heteroatom. The heteroatom is preferably an oxygen atom, a nitrogen atom, a sulfur atom, a halogen atom, etc., and more preferably an oxygen atom. The hydrocarbyl group particularly preferably has 6 to 30 carbon atoms, from the viewpoint of obtaining high resolution in fine pattern formation.

In the general formula (Xa-1-1), the hydrocarbyl group having 1 to 35 carbon atoms represented by R^{fa1} may be saturated or unsaturated, and may be linear, branched, or cyclic. Specific examples thereof include alkyl groups having 1 to 35 carbon atoms, such as methyl, ethyl, n-propyl, isopropyl, n-butyl, isobutyl, sec-butyl, tert-butyl, pentyl, neopentyl, hexyl, heptyl, 2-ethylhexyl, nonyl, undecyl, tridecyl, pentadecyl, heptadecyl, and icosyl groups; cyclic saturated hydrocarbyl groups having 3 to 35 carbon atoms, such as cyclopentyl, cyclohexyl, 1-adamantyl, 2-adamantyl, 1-adamantylmethyl, norbornyl, norbornylmethyl, tricyclodecyl, tetracyclododecyl, tetracyclododecylmethyl, and dicyclohexylmethyl groups; unsaturated aliphatic hydrocarbyl groups having 2 to 35 carbon atoms, such as 2-propenyl and 3-cyclohexenyl groups; aryl groups having 6 to 35 carbon atoms, such as phenyl, 1-naphthyl, 2-naphthyl, and 9-fluorenyl groups; aralkyl groups having 7 to 35 carbon atoms, such as benzyl and diphenylmethyl groups; and groups obtained by combining these groups.

Furthermore, some or all of the hydrogen atoms of the hydrocarbyl groups may be substituted with a group containing a heteroatom such as an oxygen atom, a sulfur atom, a nitrogen atom, or a halogen atom, and some of the —CH₂— of the hydrocarbyl groups may be substituted with a group containing a heteroatom such as an oxygen atom, a sulfur atom, or a nitrogen atom, and as a result, they may contain a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group. Specific examples of hydrocarbyl groups containing heteroatoms include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, and 3-oxocyclohexyl groups.

In the general formula (Xa-1-1), L^{a1} represents a single bond, an ether bond, an ester bond, a sulfonate ester bond, a carbonate bond, or a carbamate bond, but from the viewpoint of synthesis, it preferably represents an ether bond or an ester bond, and further preferably an ester bond.

Specific examples of the anion represented by the general formula (Xa-1) include, but are not limited to, the following. In the following formulae, Q¹ is as defined above, and Ac represents an acetyl group.

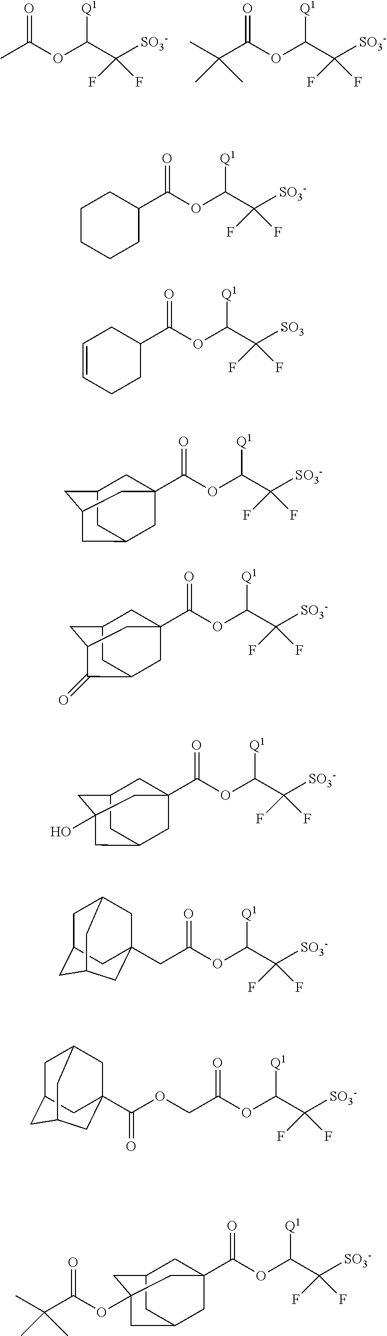

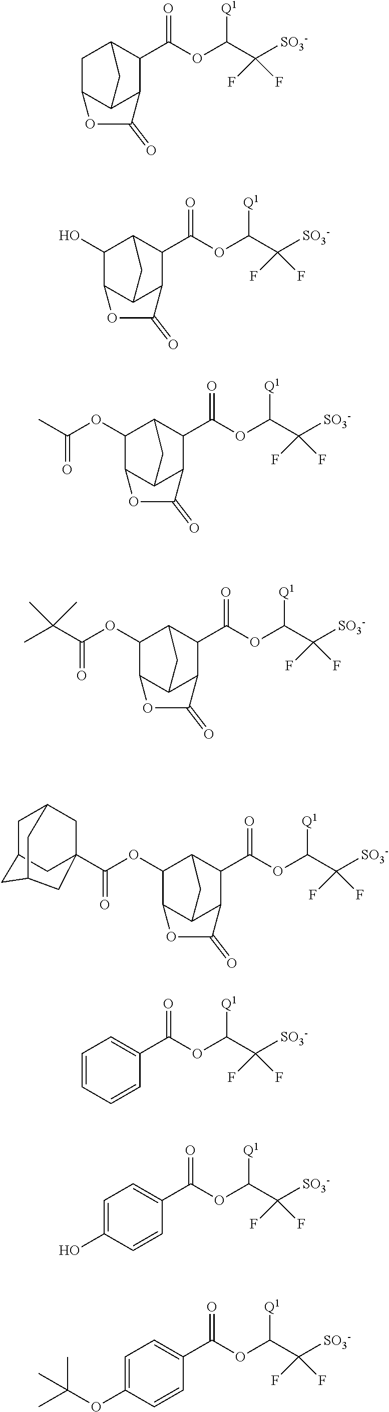

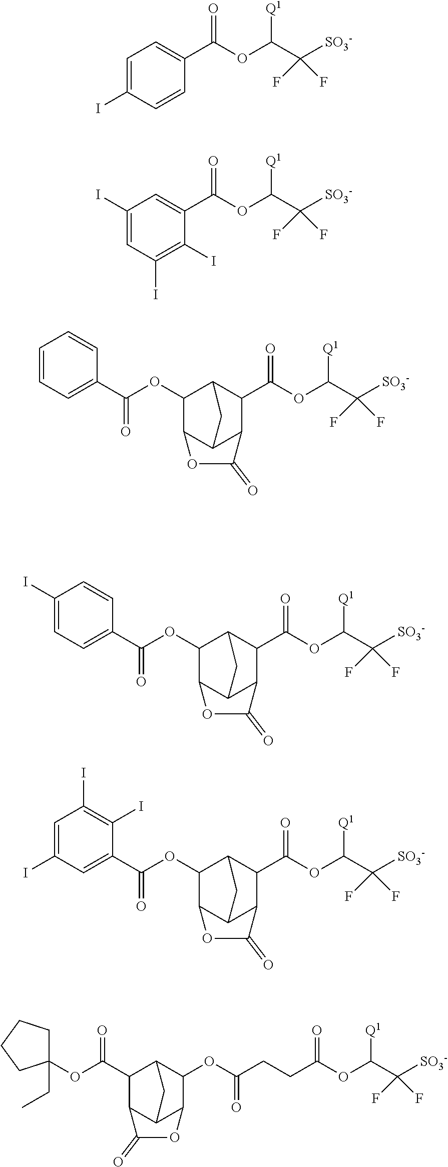

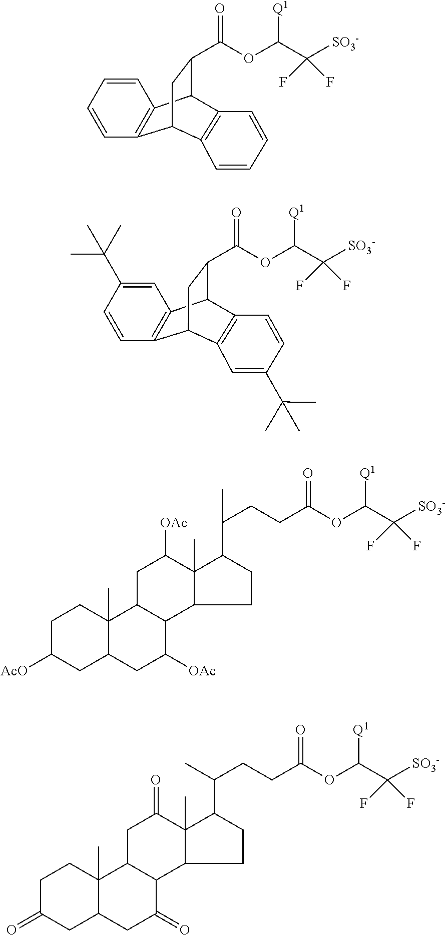

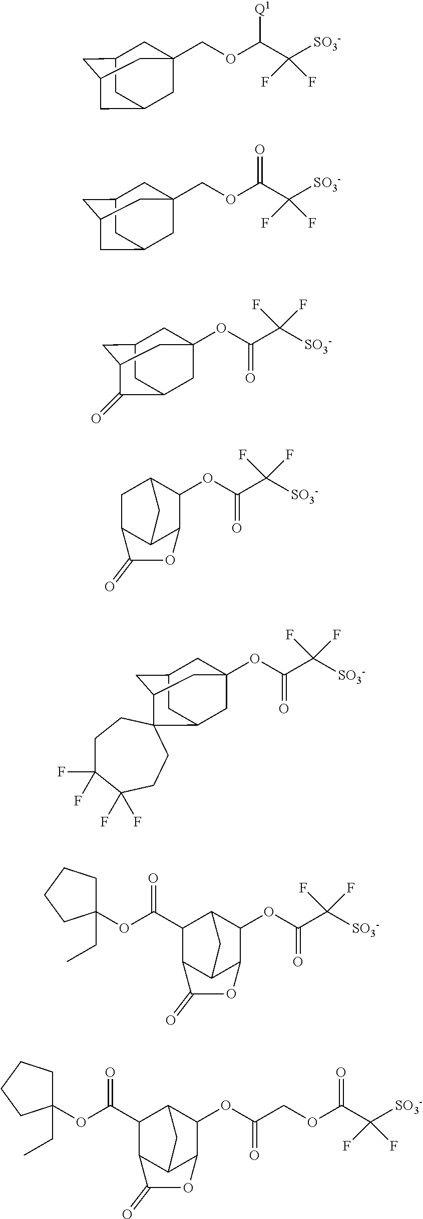

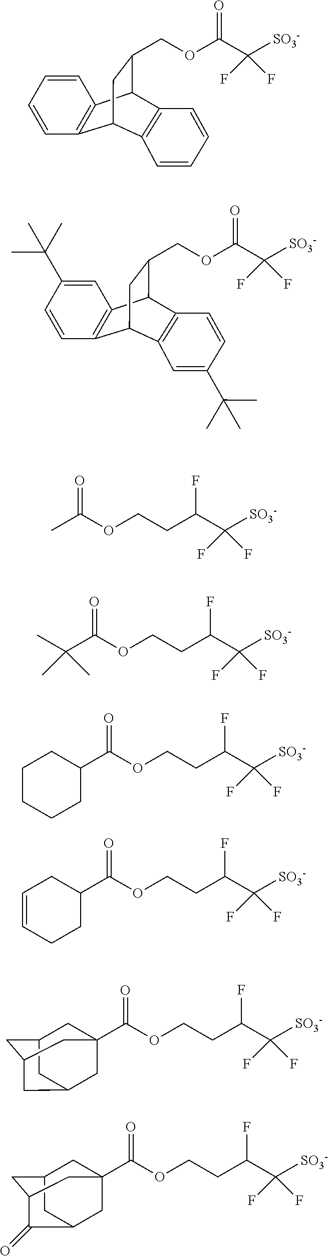

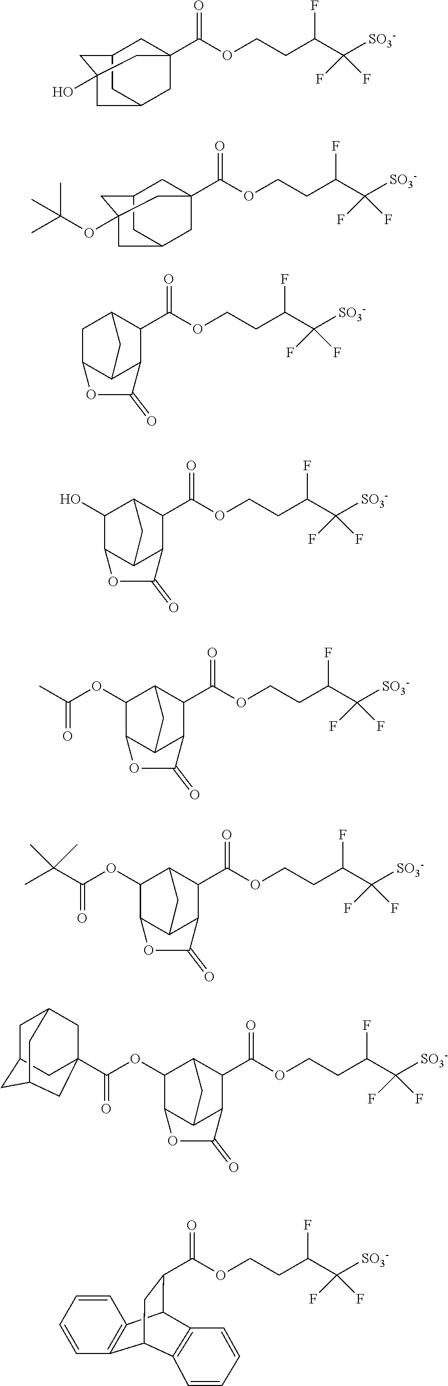

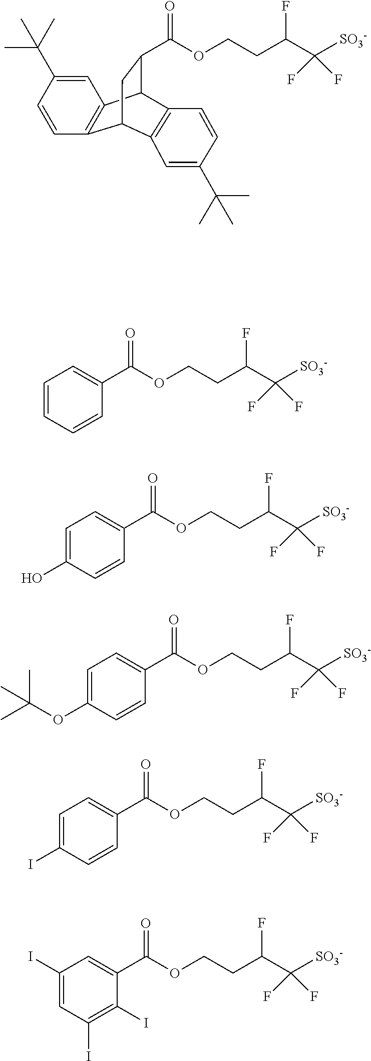

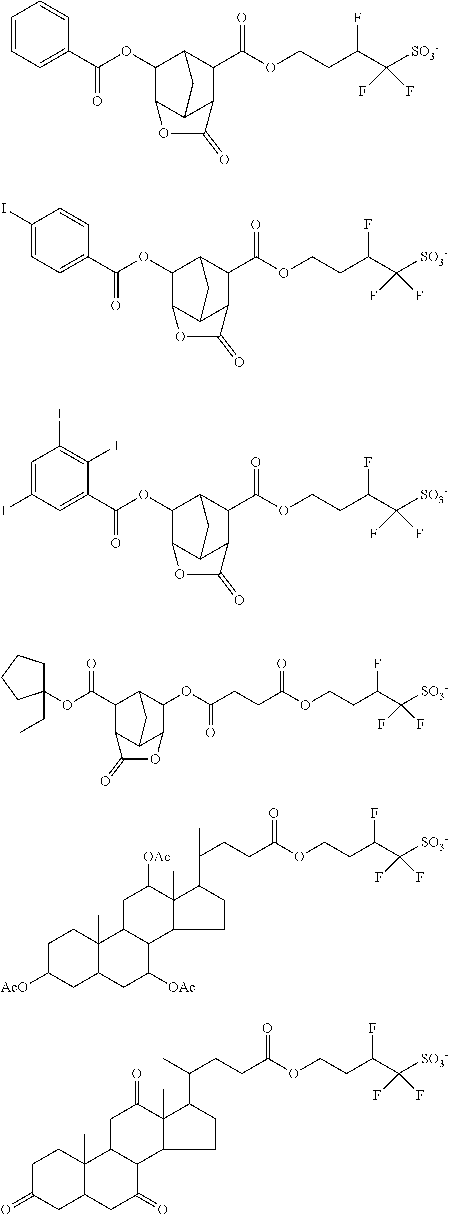

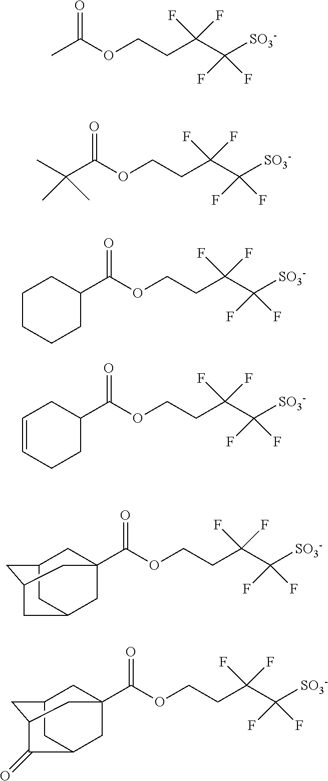

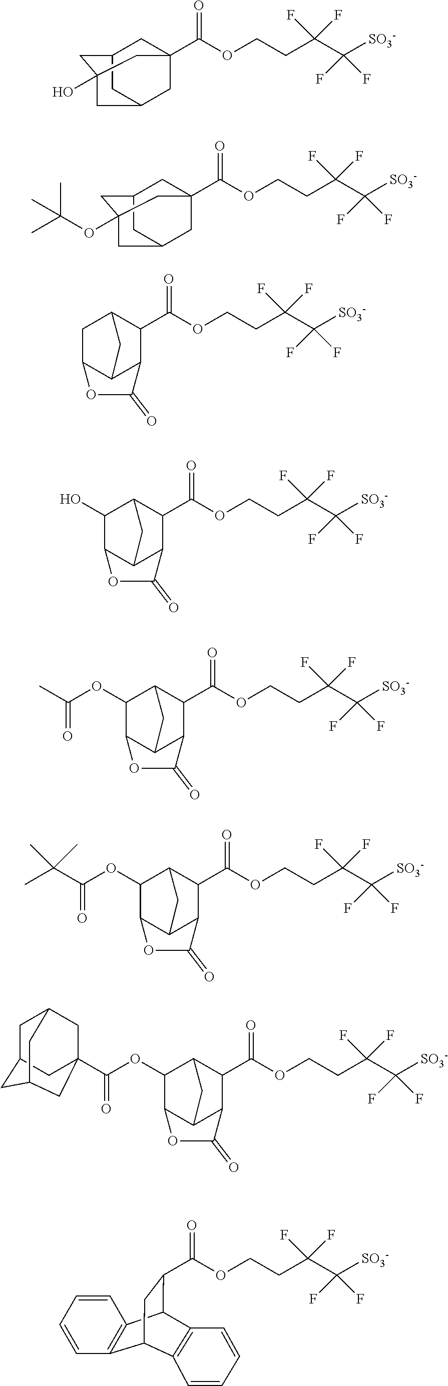

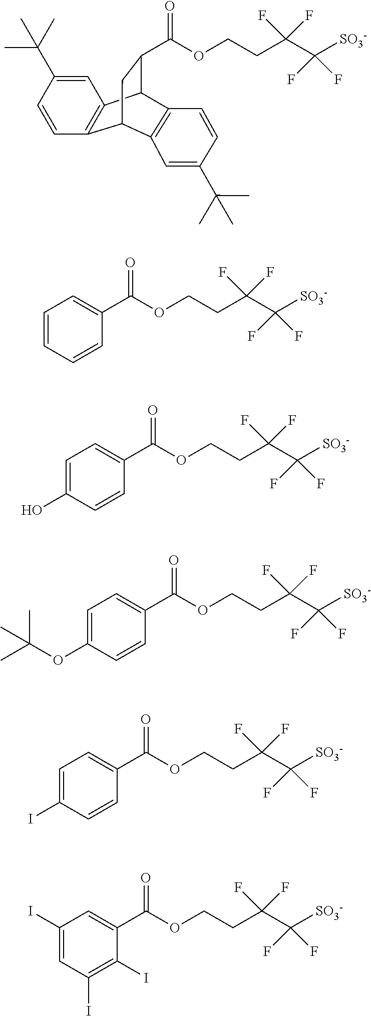

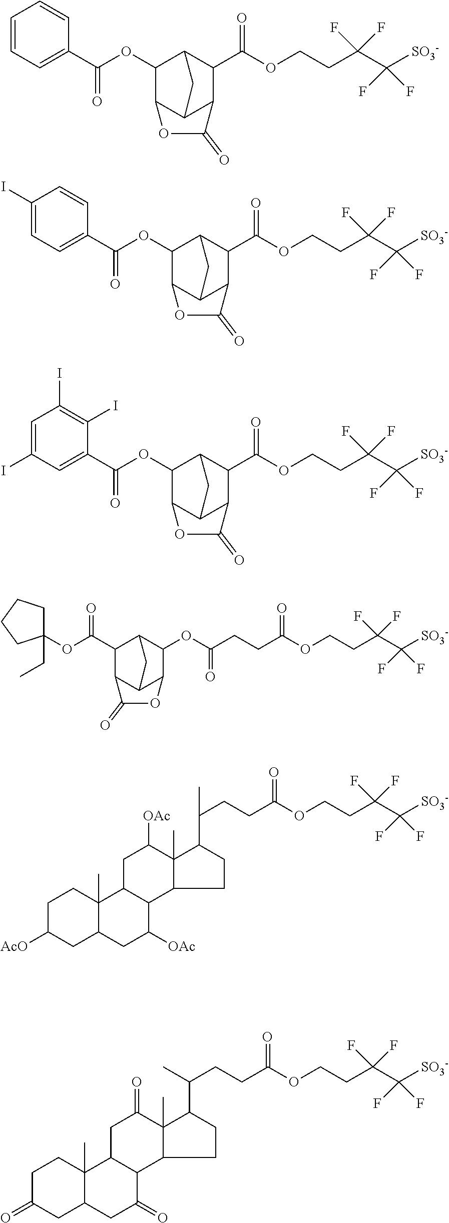

In the general formula (Xa-2), R^{fb1} and R^{fb2} each independently represent a fluorine atom, or a hydrocarbyl group having 1 to 40 carbon atoms and optionally containing a heteroatom. The hydrocarbyl group may be saturated or unsaturated, and may be linear, branched, or cyclic. Specific examples thereof include the same ones as those given as examples of the hydrocarbyl group represented by R^{fa1} in the general formula (Xa-1-1). R^{fb1} and R^{fb2} preferably represent a fluorine atom or a linear fluorinated alkyl group having 1 to 4 carbon atoms. Furthermore, R^{fb1} and R^{fb2} may be bonded to each other to form a ring with the group (—CF₂—SO₂—N⁻SO₂—CF₂—) to which they are bonded, in which case the group obtained by bonding R^{fb1} and R^{fb2} to each other is preferably a fluorinated ethylene group or a fluorinated propylene group.

In the general formula (Xa-3), R^{fc1}, R^{fc2}, and R^{fc3} each independently represent a fluorine atom, or a hydrocarbyl group having 1 to 40 carbon atoms and optionally containing a heteroatom. The hydrocarbyl group may be saturated or unsaturated, and may be linear, branched, or cyclic. Specific examples thereof include the same ones as those given as examples of the hydrocarbyl group represented by R^{fa1} in the general formula (Xa-1-1). R^{fc1}, R^{fc2}, and R^{fc3} preferably represent a fluorine atom or a linear fluorinated alkyl group having 1 to 4 carbon atoms. Furthermore, R^{fc1} and R^{fc2} may be bonded to each other to form a ring with the group (—CF₂—SO₂—C⁻SO₂—CF₂—) to which they are bonded, in which case the group obtained by bonding R^{fc1} and R^{fc2} to each other is preferably a fluorinated ethylene group or a fluorinated propylene group.

In the general formula (Xa-4), R^{fd} represents a hydrocarbyl group having 1 to 40 carbon atoms and optionally containing a heteroatom. The hydrocarbyl group may be saturated or unsaturated, and may be linear, branched, or cyclic. Specific examples thereof include the same ones as those given as examples of the hydrocarbyl group represented by R^{fa1} in the general formula (Xa-1-1).

Specific examples of the anion represented by the general formula (Xa-4) include, but are not limited to, the following.

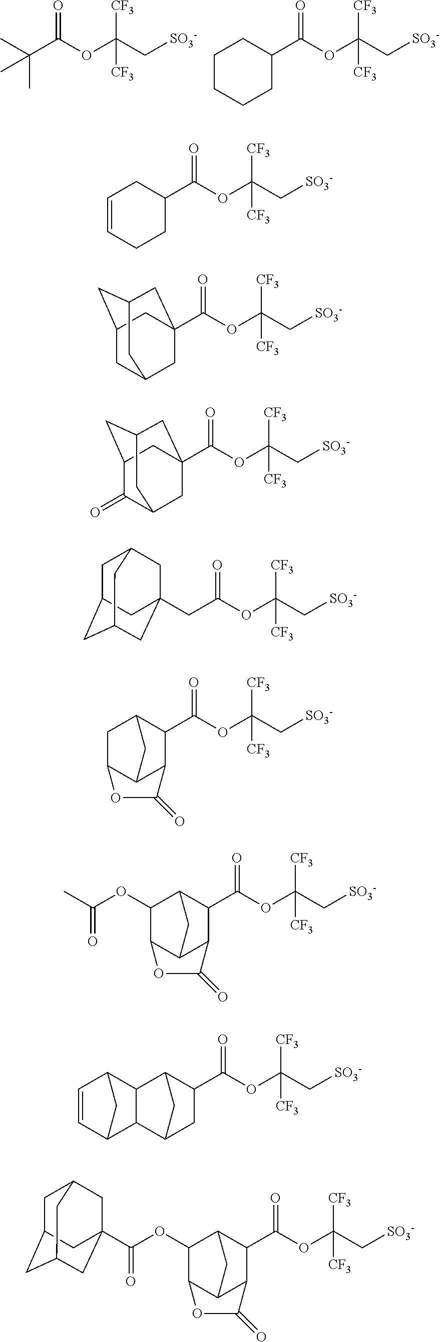

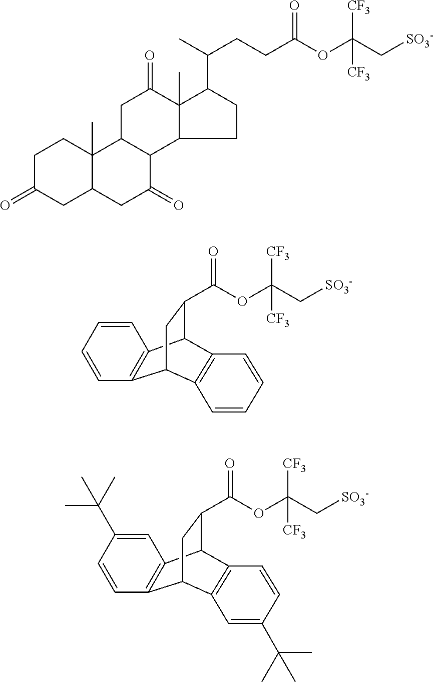

Further examples of the anion of the strong acid include anions having an aromatic ring substituted with an iodine atom or a bromine atom. Specific examples of such anions include those represented by the following general formula (Xa-5).

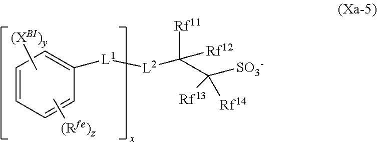

In the general formula (Xa-5), “x” represents 1, 2, or 3. “y” represents an integer from 1 to 5. “z” represents an integer from 0 to 3. Note, 1≤y+z≤5. “y” preferably represents 1, 2, or 3, and more preferably 2 or 3. “z” preferably represents 0, 1, or 2.

In the general formula (Xa-5), X^{BI} is an iodine atom or a bromine atom, and they may be the same as or different from each other when “x” and/or “y” are 2 or more.

In the general formula (Xa-5), L¹ is a single bond, an ether bond, an ester bond, or a saturated hydrocarbylene group having 1 to 6 carbon atoms and optionally containing an ether bond or an ester bond. The saturated hydrocarbylene group may be linear, branched, or cyclic.

In the general formula (Xa-5), L² represents a single bond or a divalent linking group having 1 to 20 carbon atoms when “x” is 1, and represents a (x+1)-valent linking group having 1 to 20 carbon atoms when “x” is 2 or 3, and the linking group may contain an oxygen atom, a sulfur atom, or a nitrogen atom.

In the general formula (Xa-5), R^{fe} represents a hydroxy group, a carboxy group, a fluorine atom, a chlorine atom, a bromine atom, or an amino group, or a hydrocarbyl group having 1 to 20 carbon atoms, a hydrocarbyloxy group having 1 to 20 carbon atoms, a hydrocarbylcarbonyl group having 2 to 20 carbon atoms, a hydrocarbyloxycarbonyl group having 2 to 20 carbon atoms, a hydrocarbylcarbonyloxy group having 2 to 20 carbon atoms, or a hydrocarbylsulfonyloxy group having 1 to 20 carbon atoms, and optionally containing a fluorine atom, a chlorine atom, a bromine atom, a hydroxy group, an amino group, or an ether bond, or —N(R^{feA}) (R^{feB}), —N(R^{feC})—C(═O)—R^{feD}, or —N(R^{feC})—C(═O)—O—R^{feD}. R^{feA} and R^{feB} each independently represent a hydrogen atom or a saturated hydrocarbyl group having 1 to 6 carbon atoms. R^{feC} represents a hydrogen atom or a saturated hydrocarbyl group having 1 to 6 carbon atoms, and optionally containing a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. R^{feD} represents an aliphatic hydrocarbyl group having 1 to 16 carbon atoms, an aryl group having 6 to 12 carbon atoms, or an aralkyl group having 7 to 15 carbon atoms, and optionally containing a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. The aliphatic hydrocarbyl group may be saturated or unsaturated, and may be linear, branched, or cyclic. The hydrocarbyl group, the hydrocarbyloxy group, the hydrocarbylcarbonyl group, the hydrocarbyloxycarbonyl group, the hydrocarbylcarbonyloxy group, and the hydrocarbylsulfonyloxy group may be linear, branched, or cyclic. When “x” and/or “z” are 2 or more, each R^{fe} may be the same as or different from each other.

Of these, R^{fe} preferably represents a hydroxy group, —N(R^{feC})—C(═O)—R^{feD}, —N(R^{feC})—C(═O)—O—R^{feD}, a fluorine atom, a chlorine atom, a bromine atom, a methyl group, or a methoxy group.

In the general formula (Xa-5), Rf¹¹ to Rf¹⁴ each independently represent a hydrogen atom, a fluorine atom, or a trifluoromethyl group, but at least one of them represents a fluorine atom or a trifluoromethyl group. In addition, Rf¹¹ and Rf¹² may combine to form a carbonyl group. In particular, it is preferable that both Rf¹³ and Rf¹⁴ represent fluorine atoms.

Specific examples of the anion represented by the general formula (Xa-5) include, but are not limited to, the following. In the formulae below, X^{BI} is as defined above.

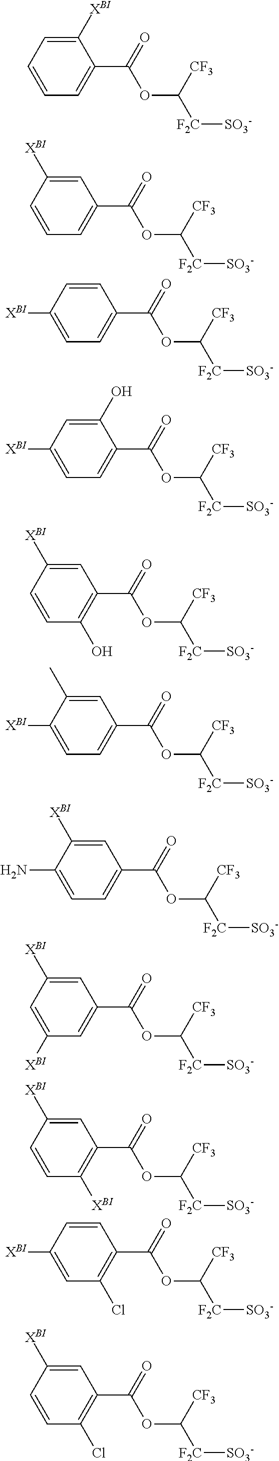

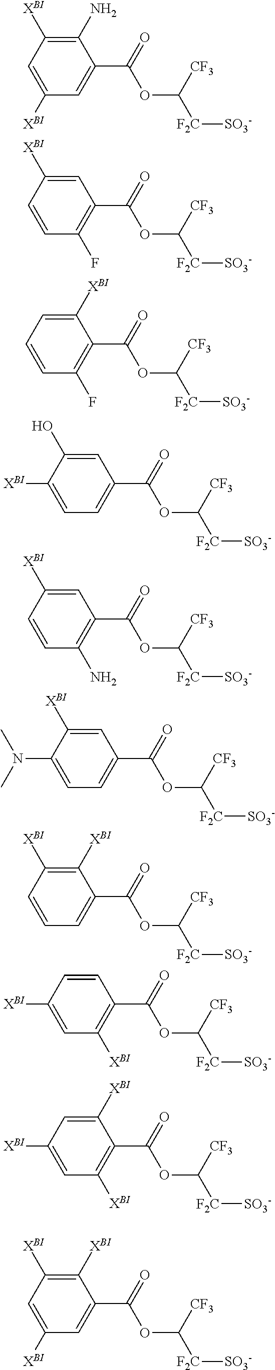

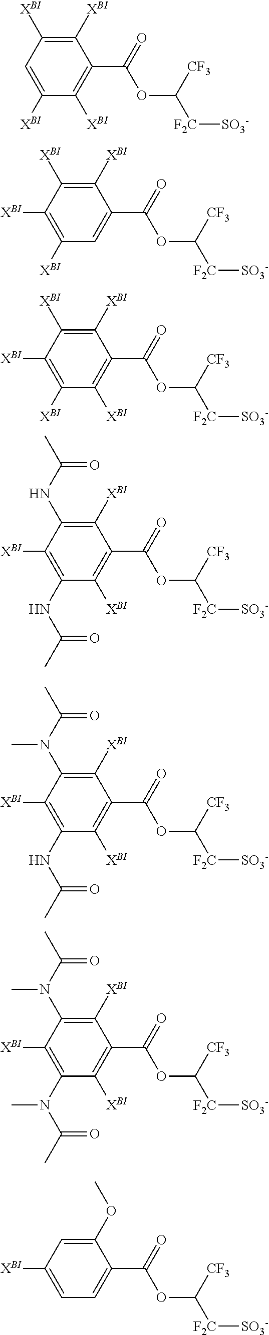

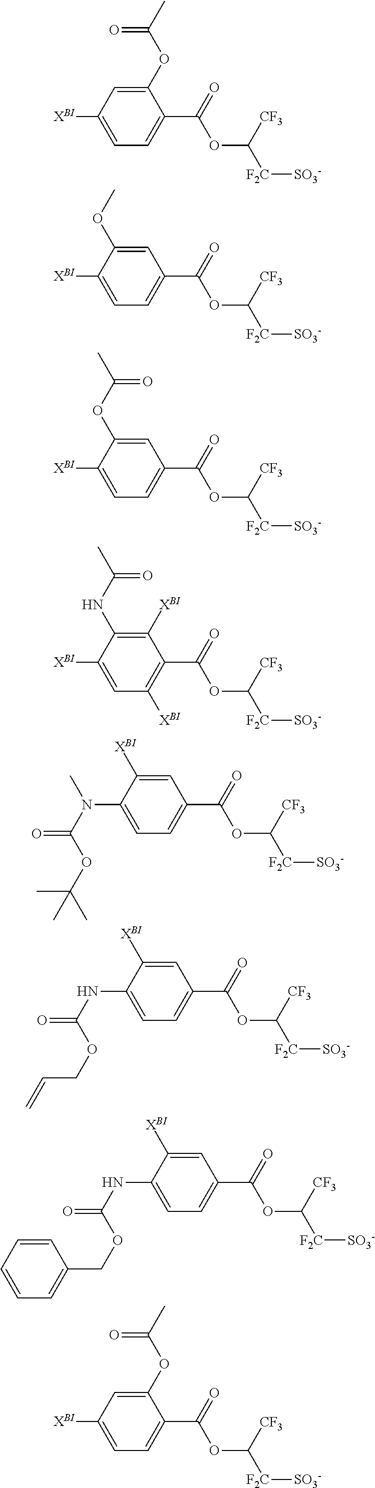

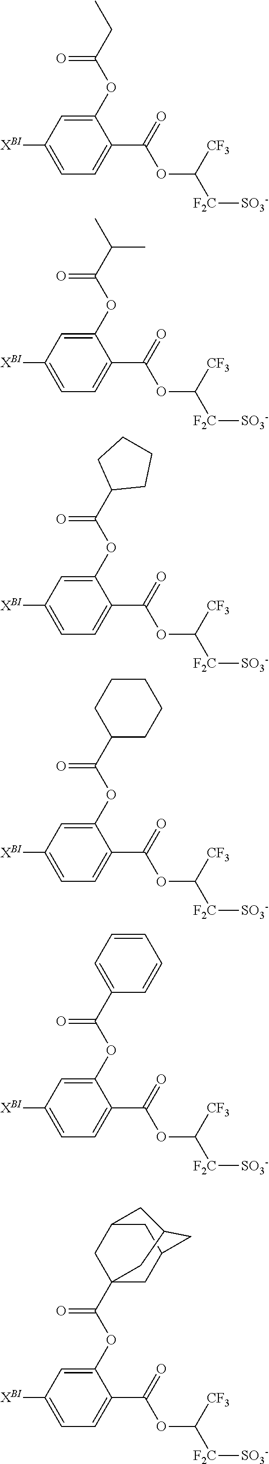

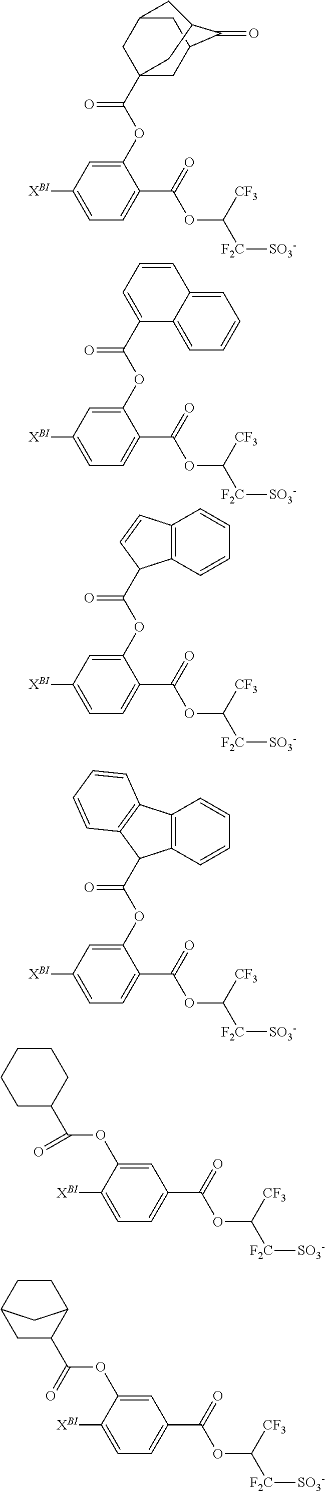

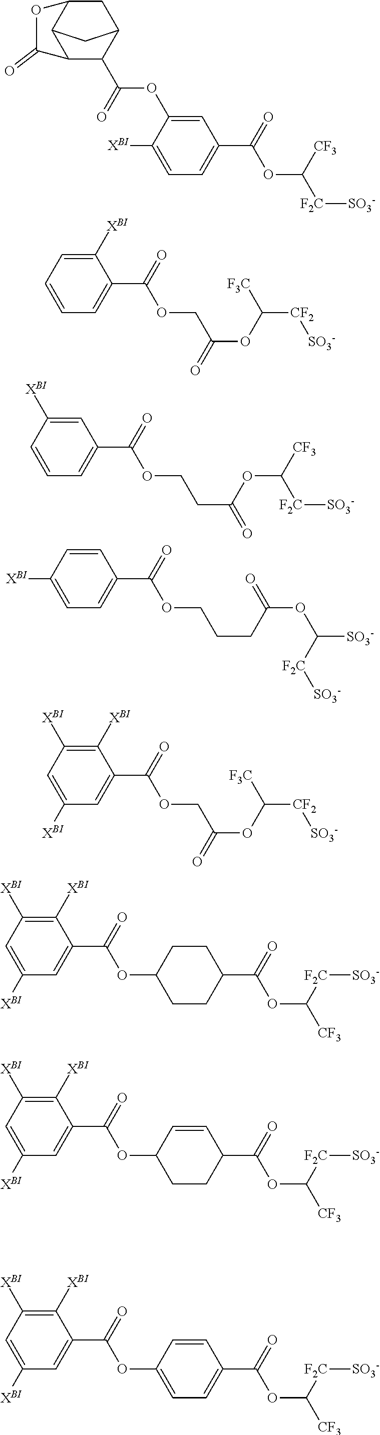

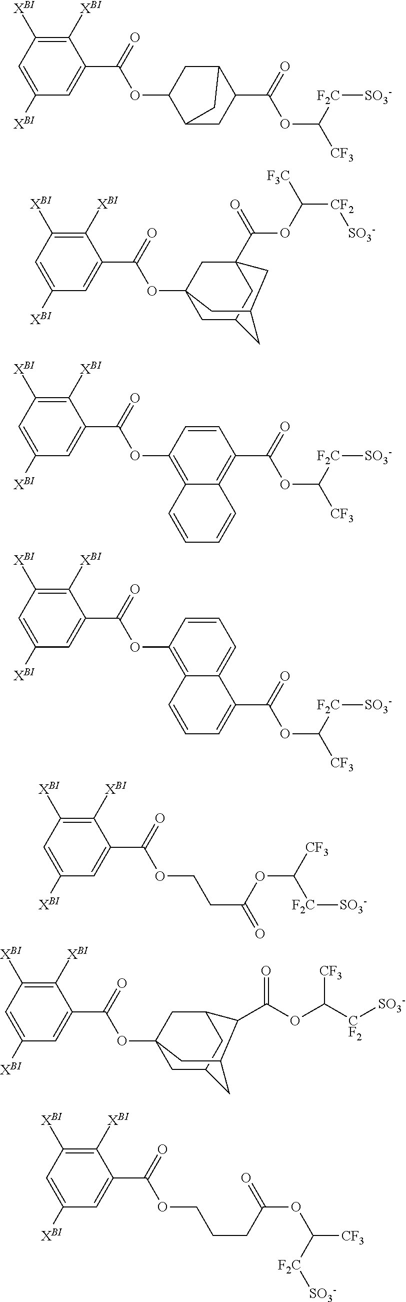

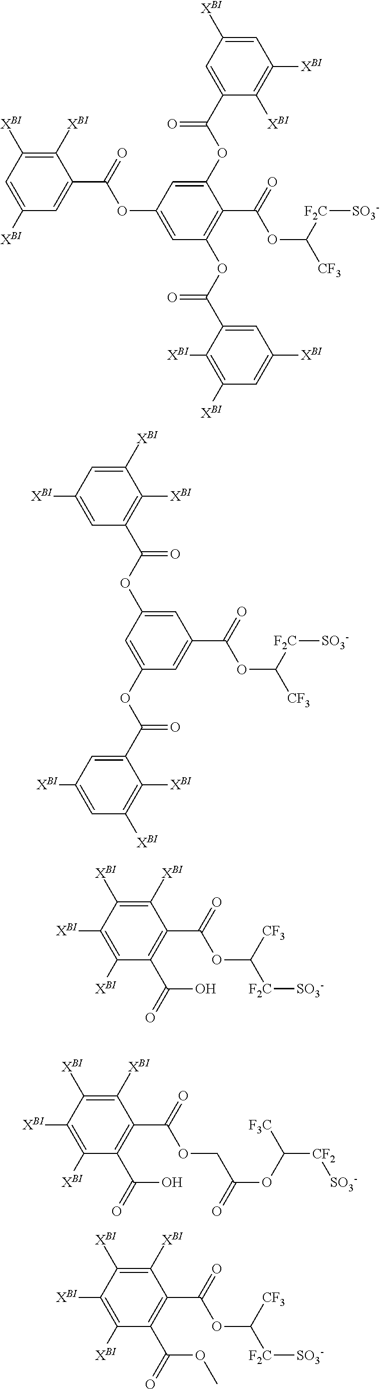

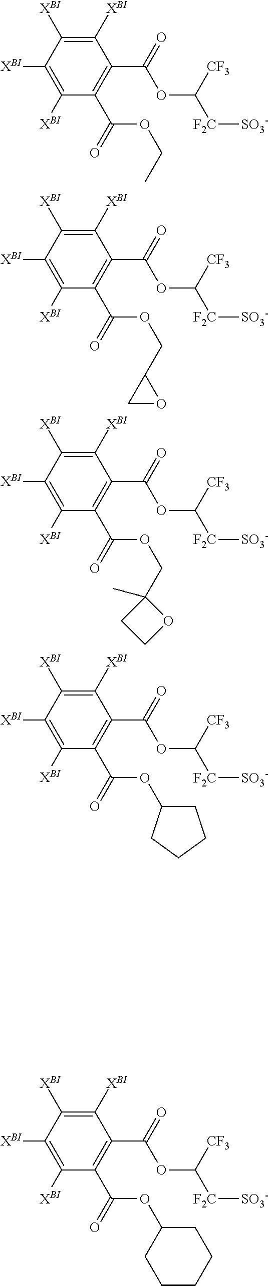

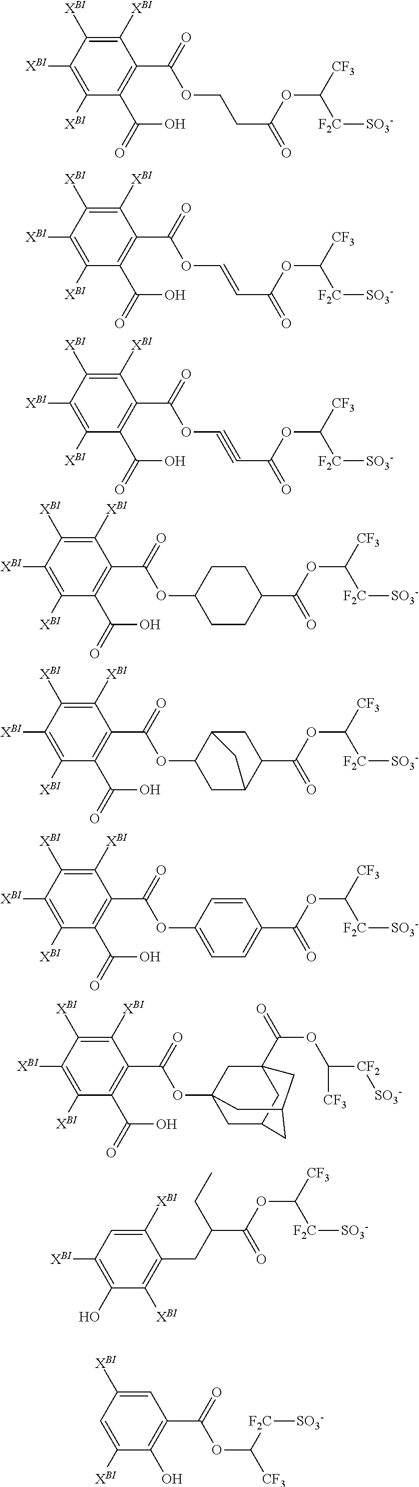

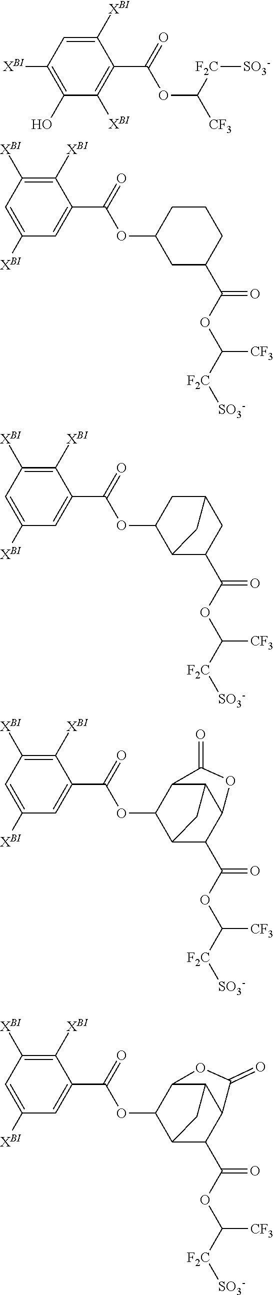

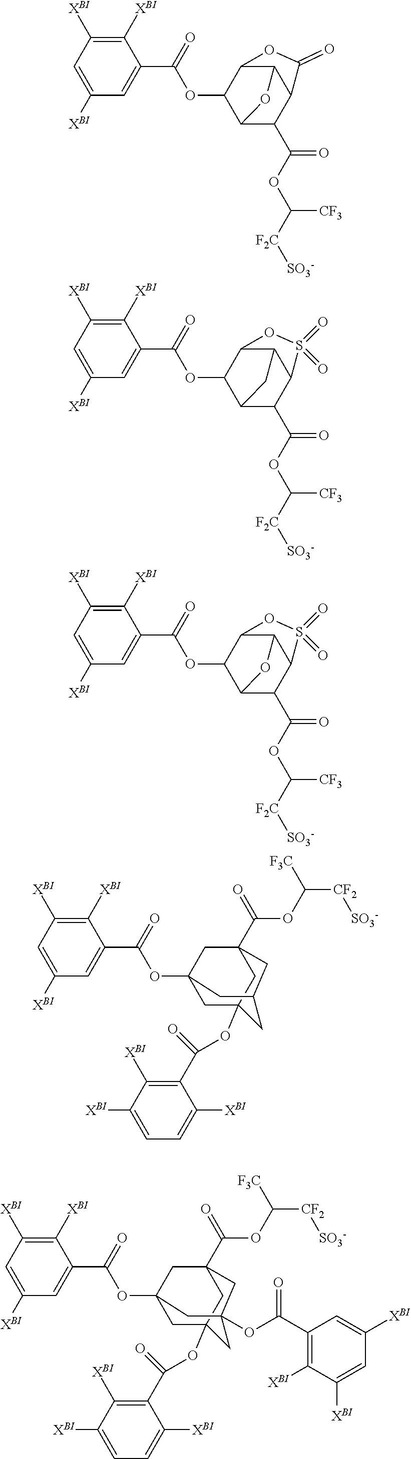

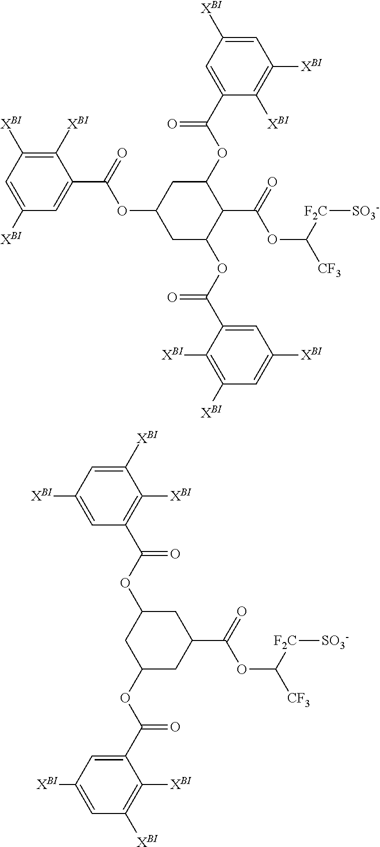

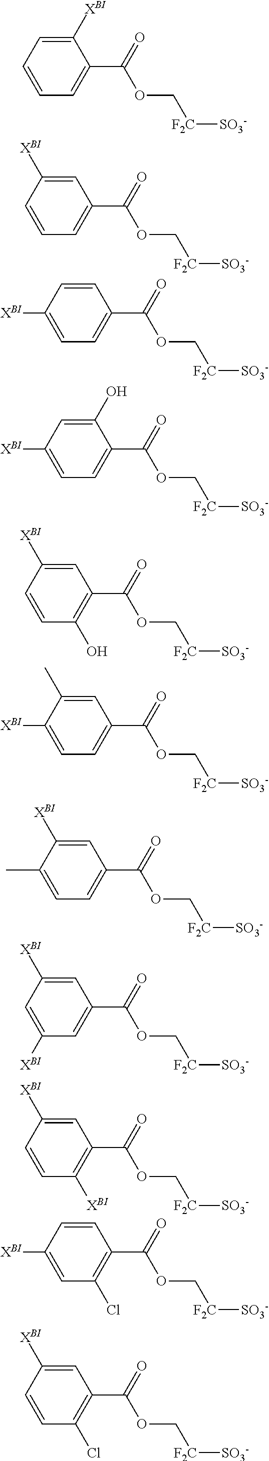

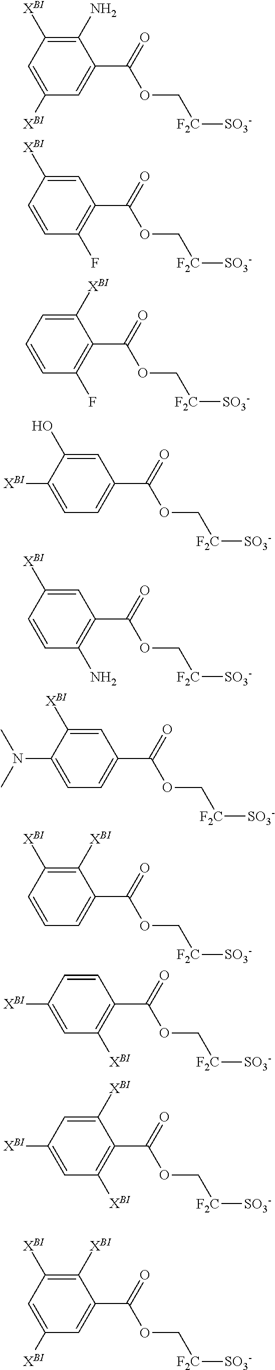

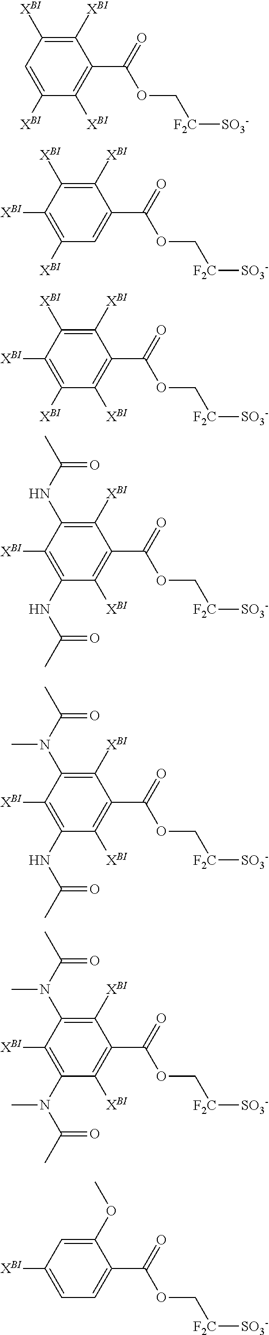

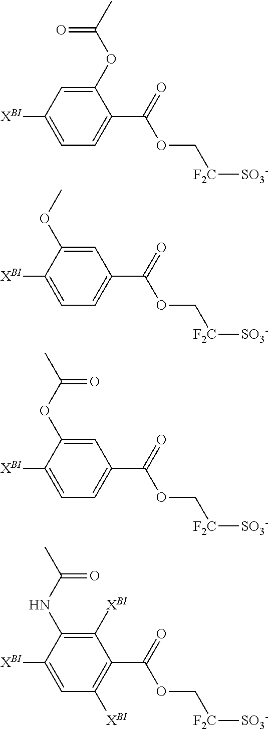

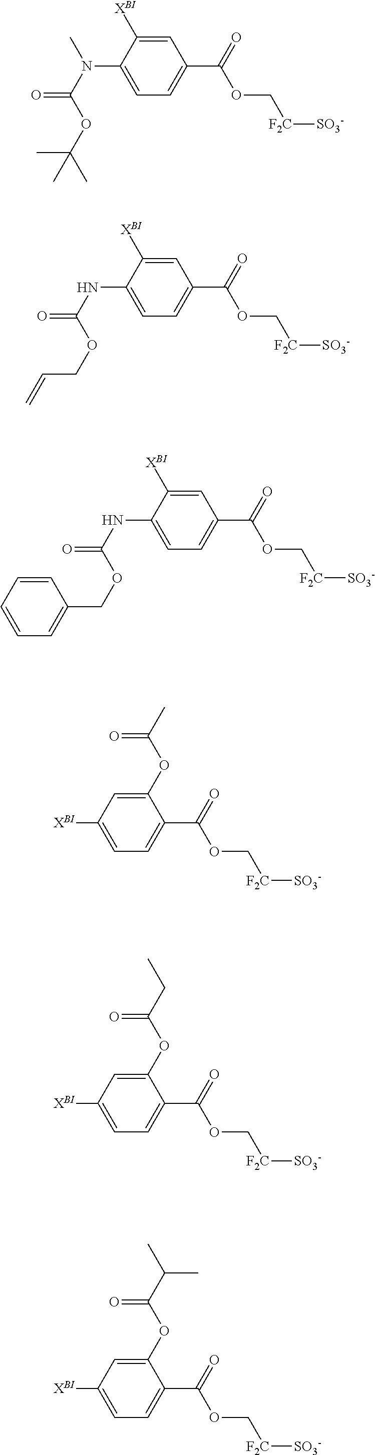

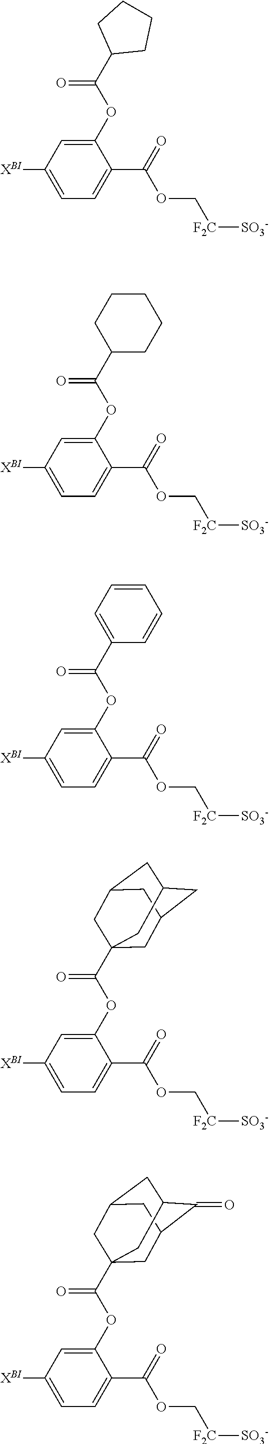

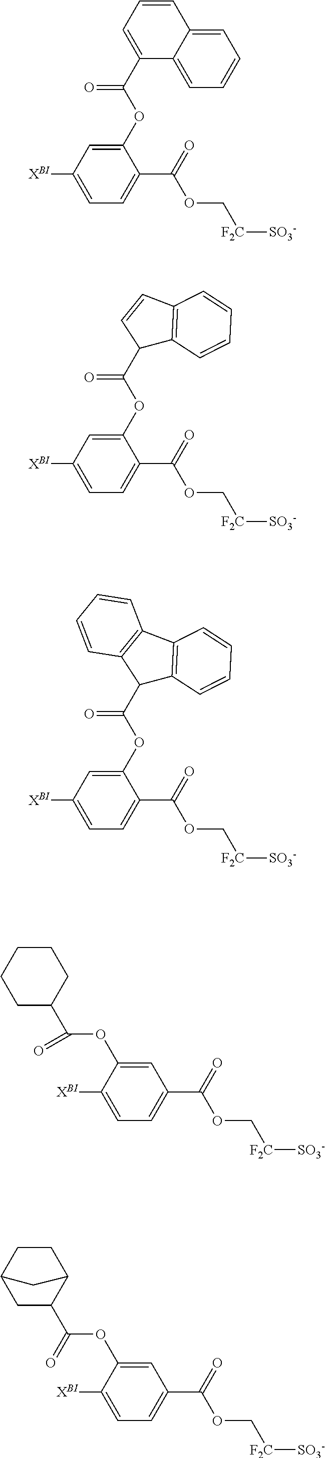

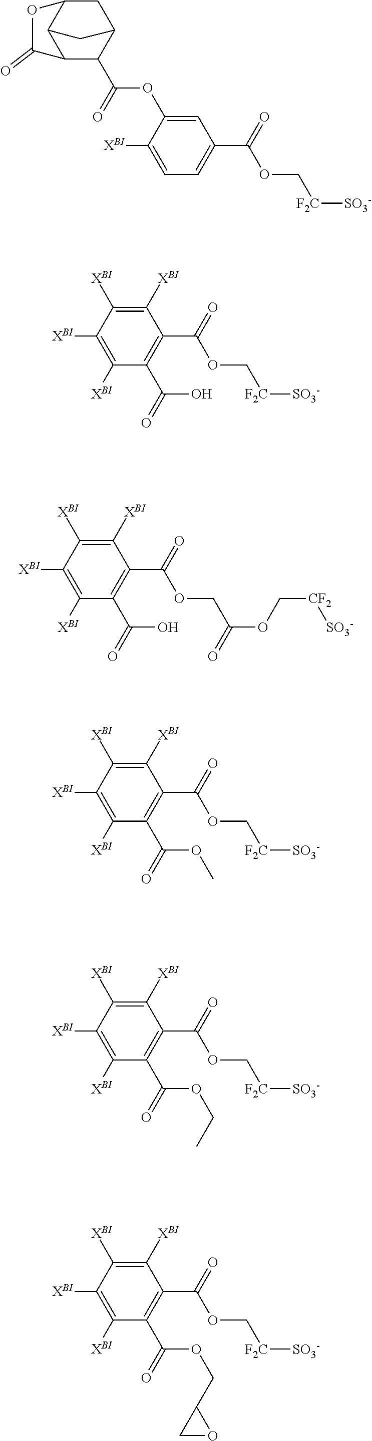

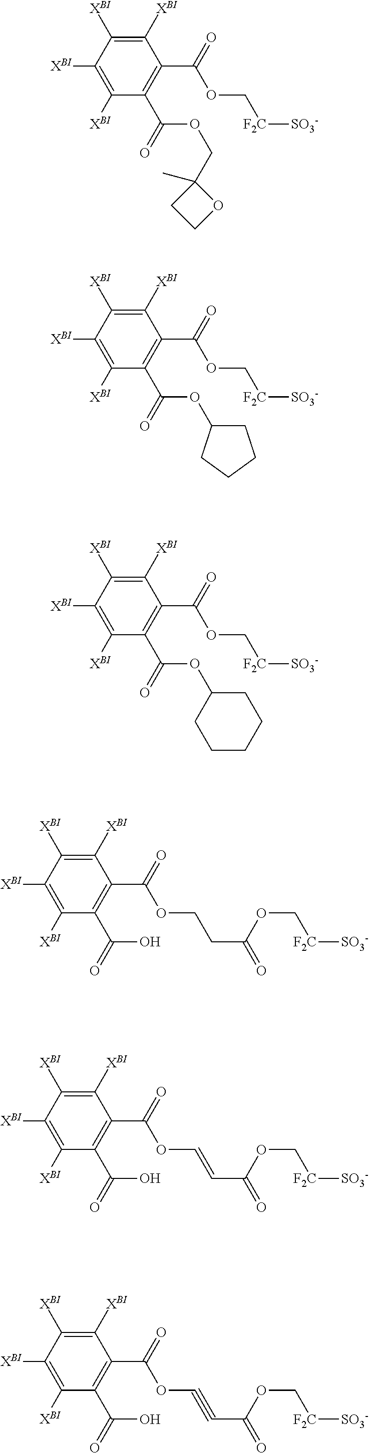

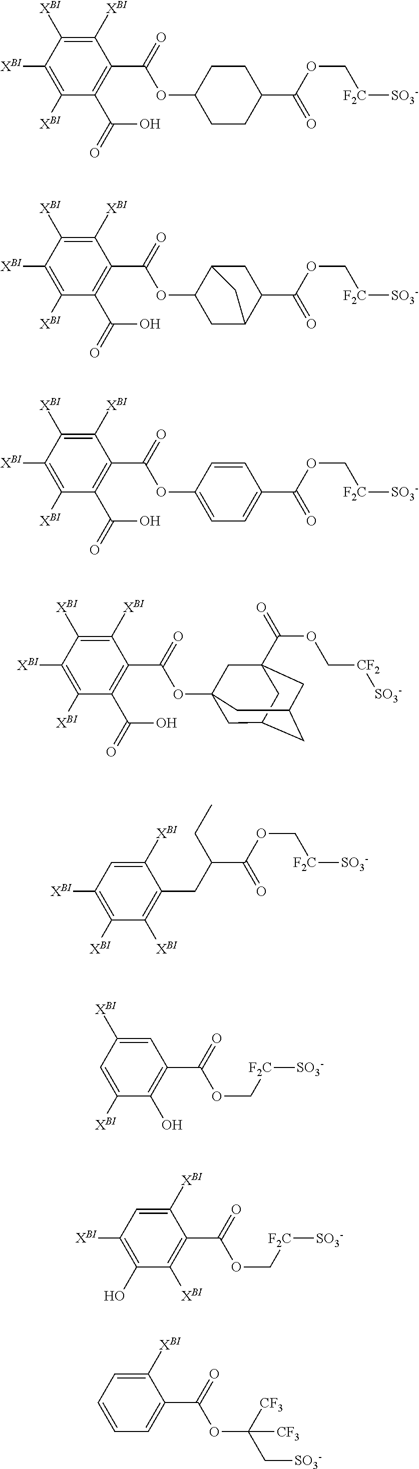

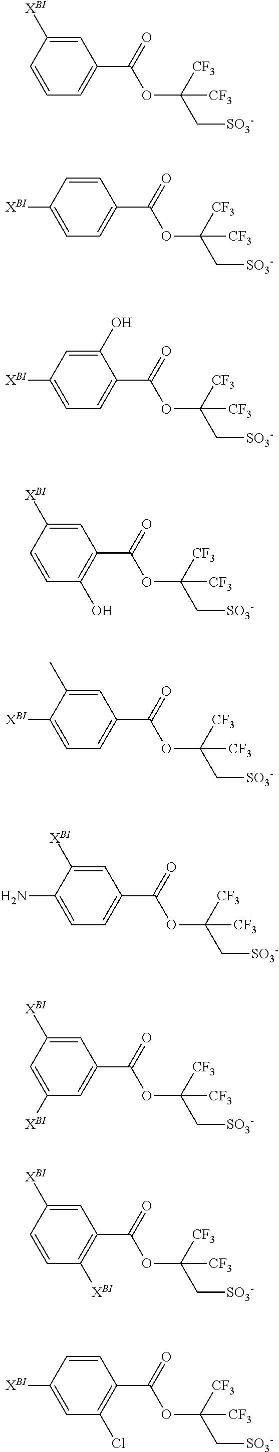

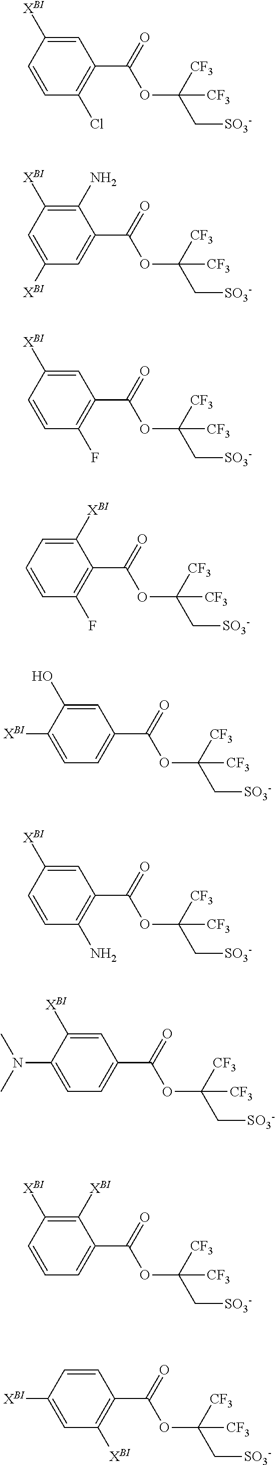

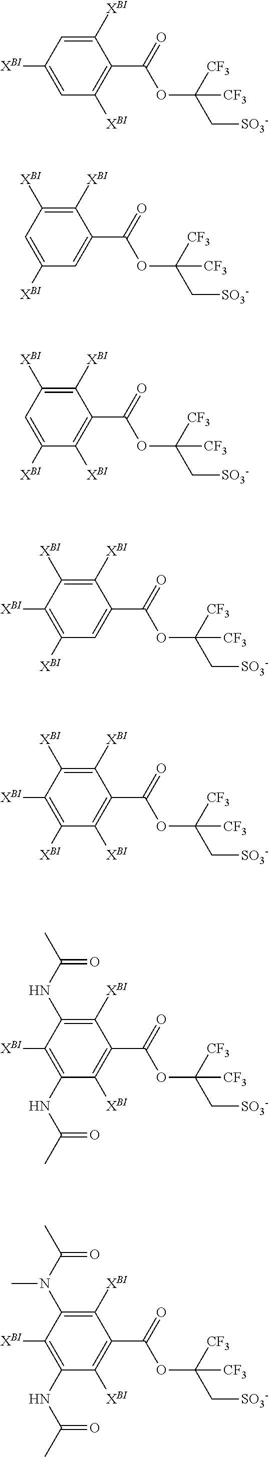

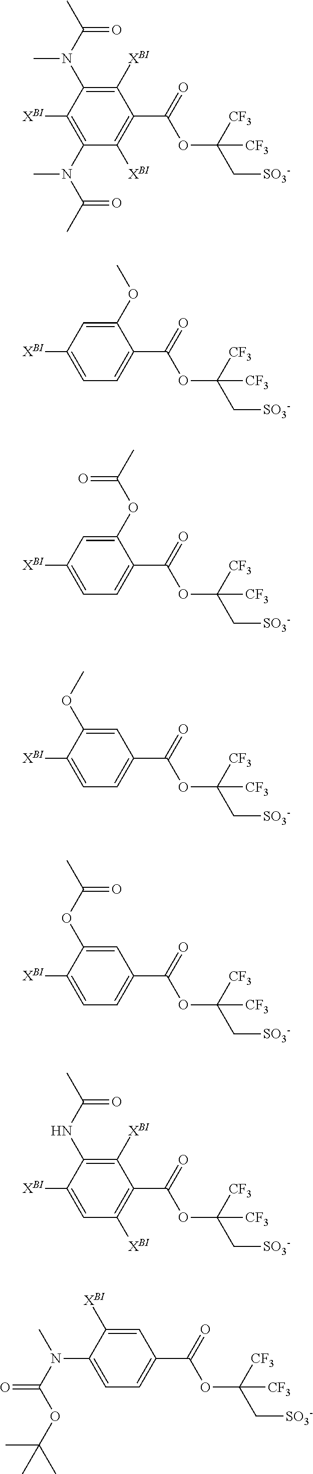

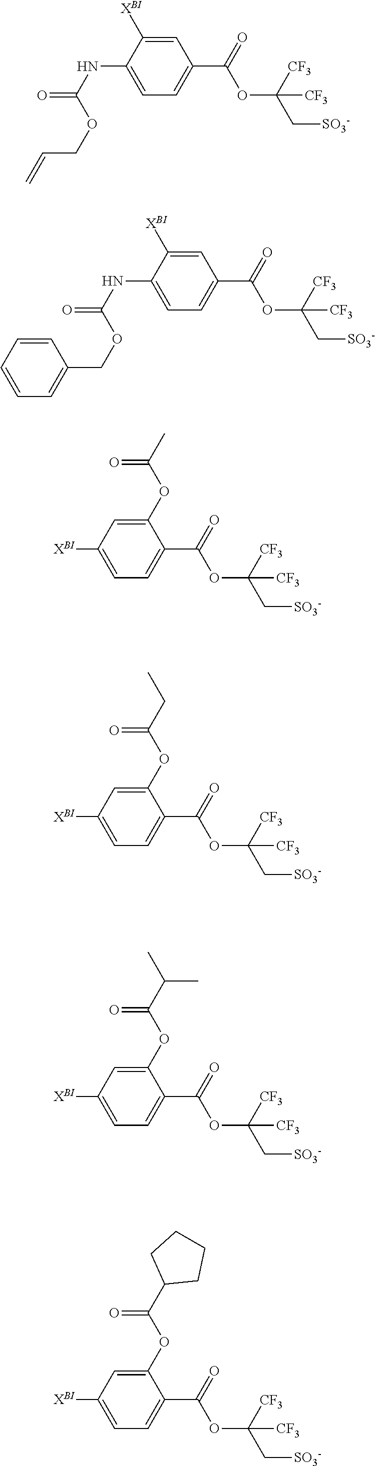

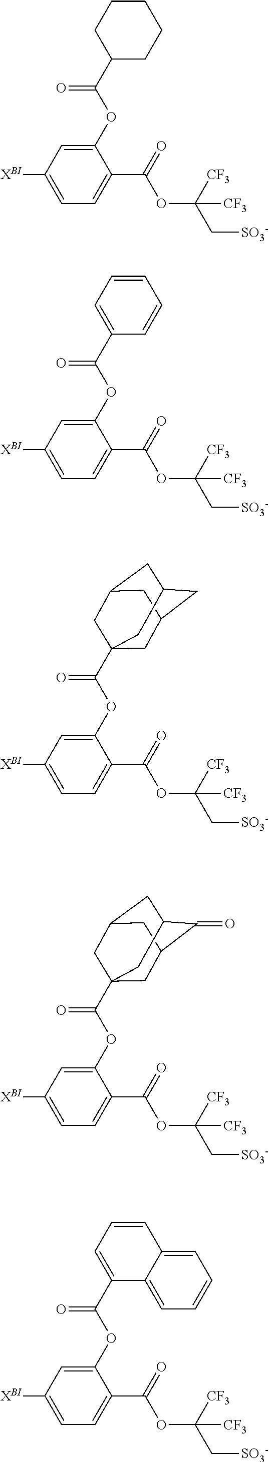

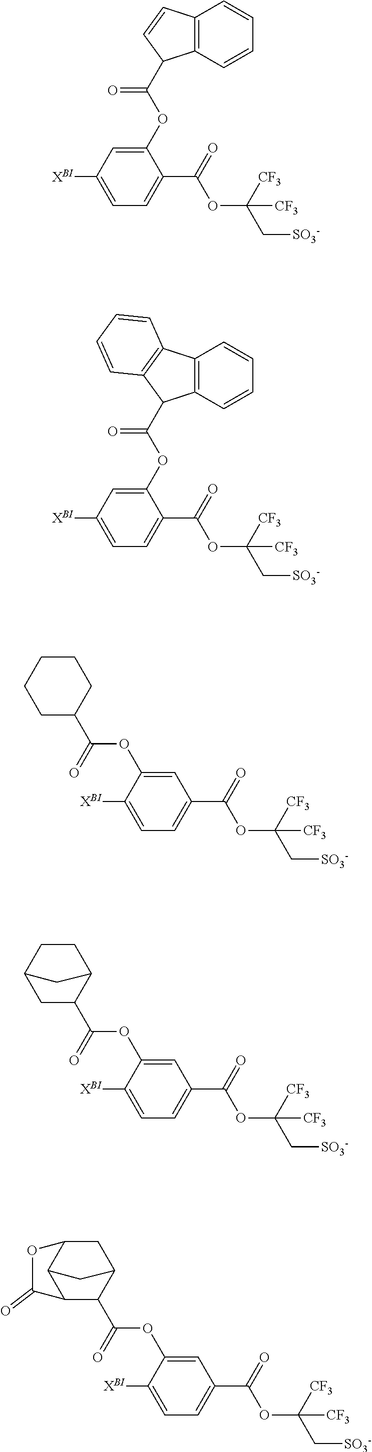

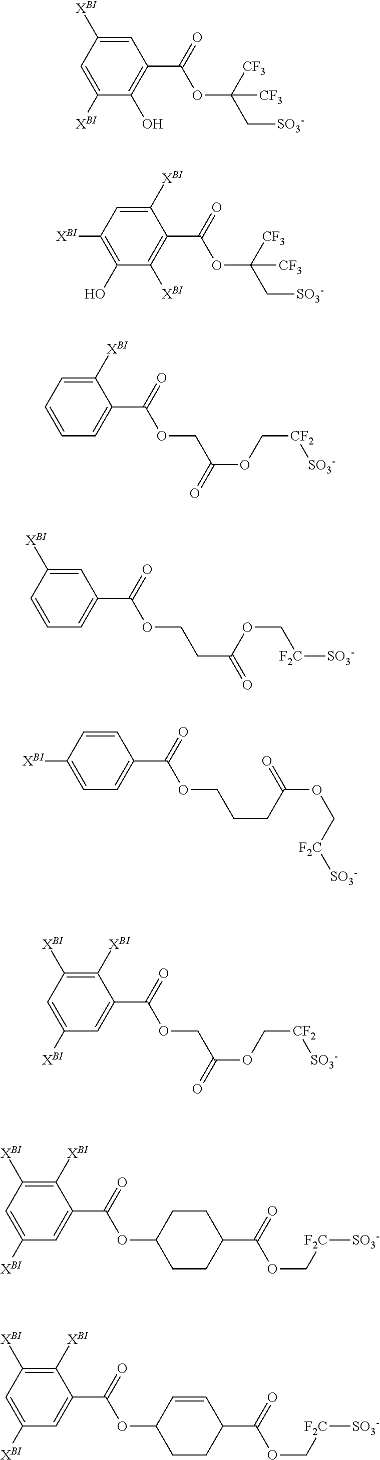

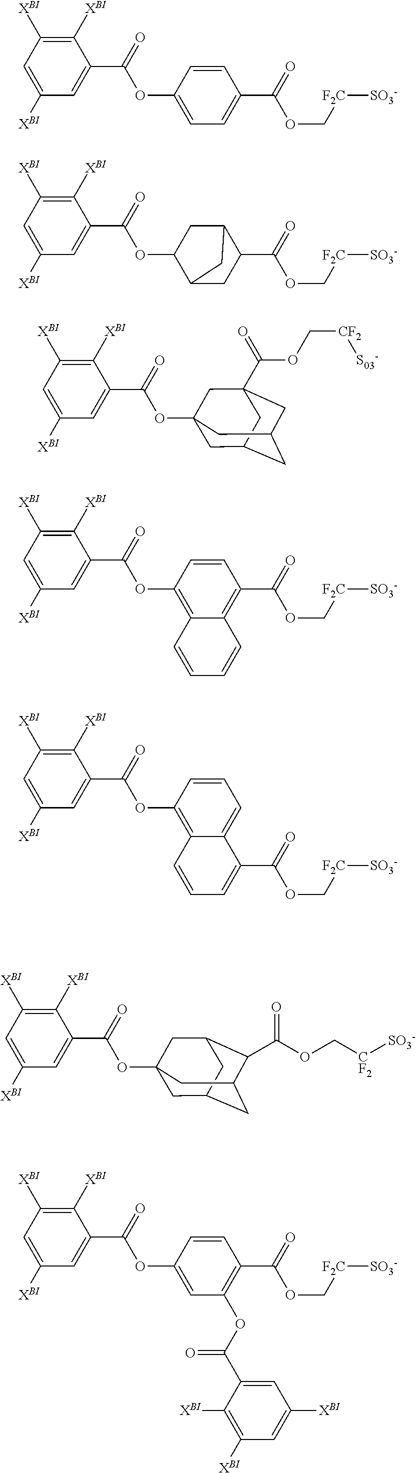

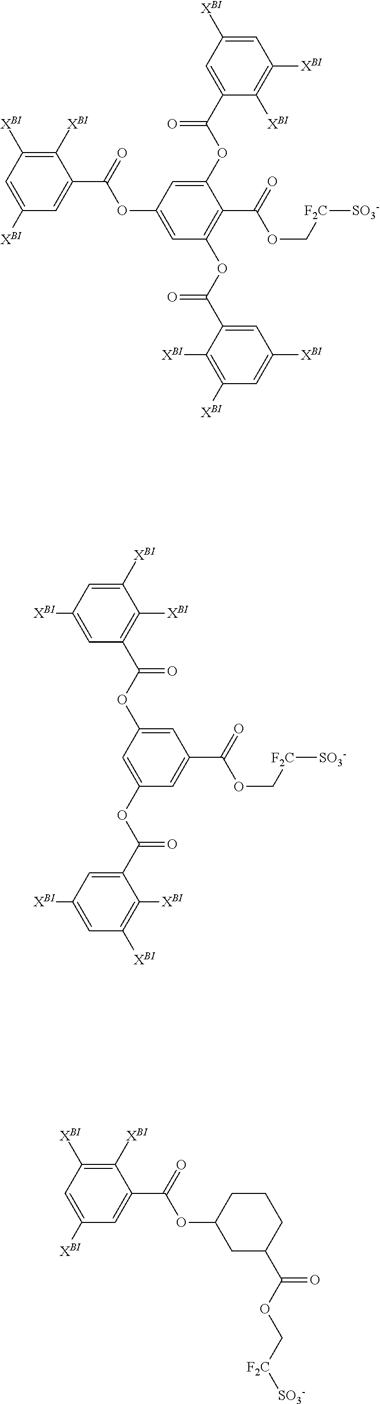

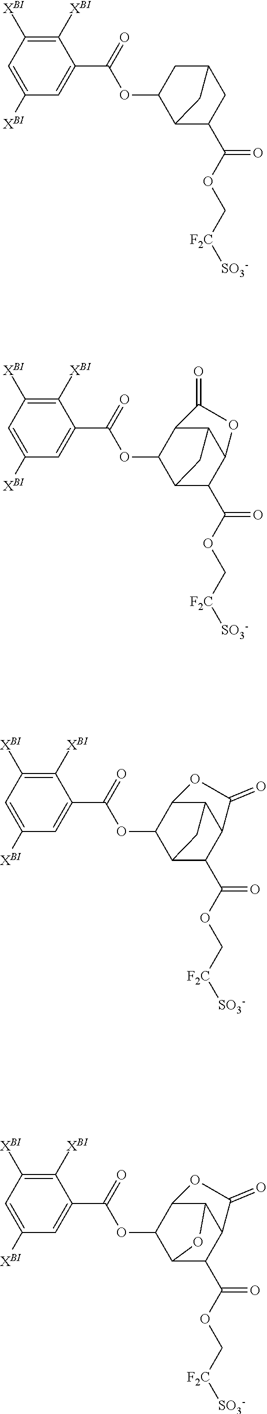

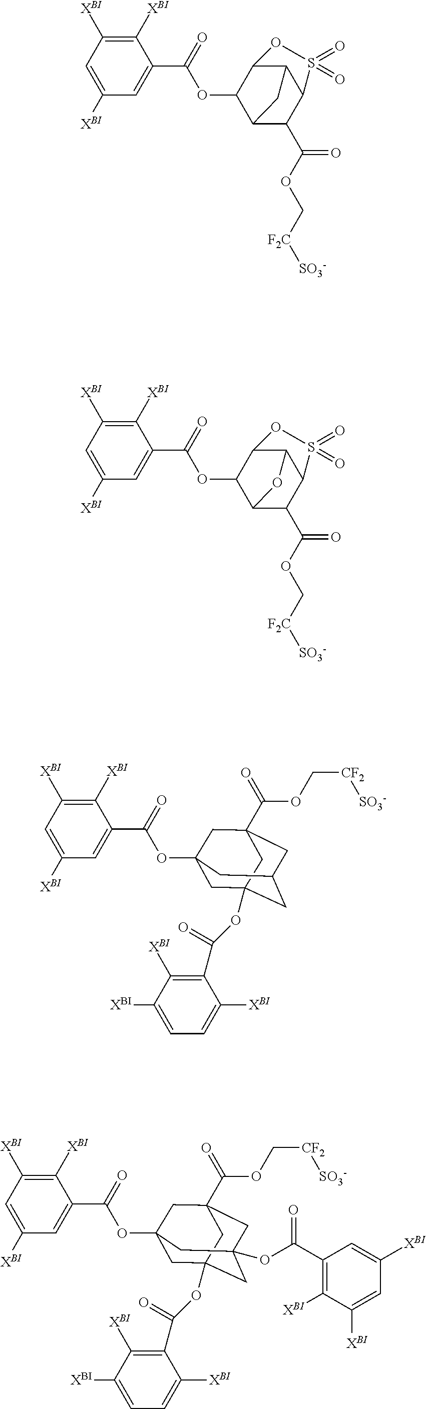

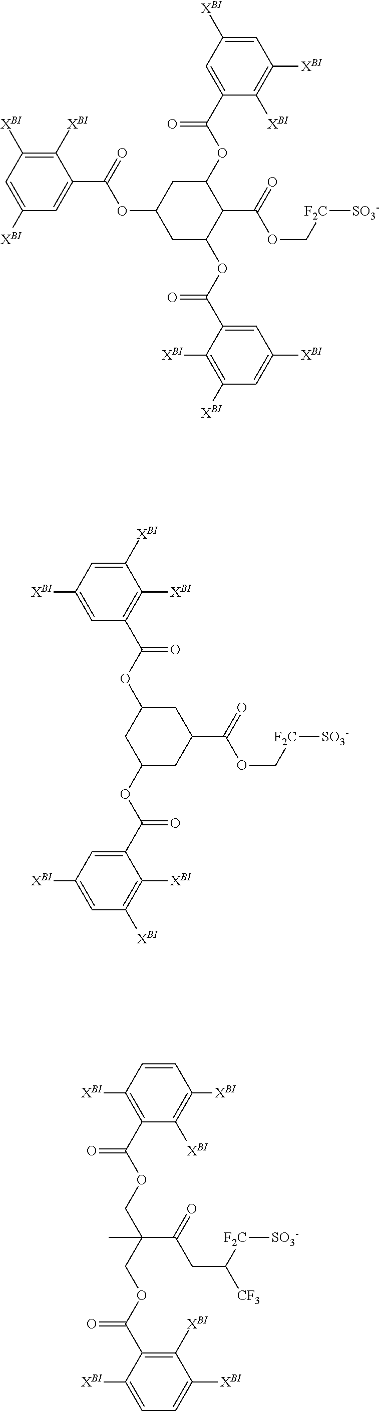

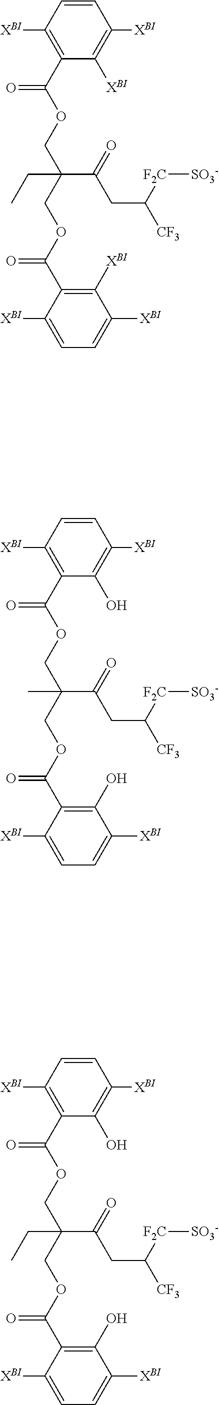

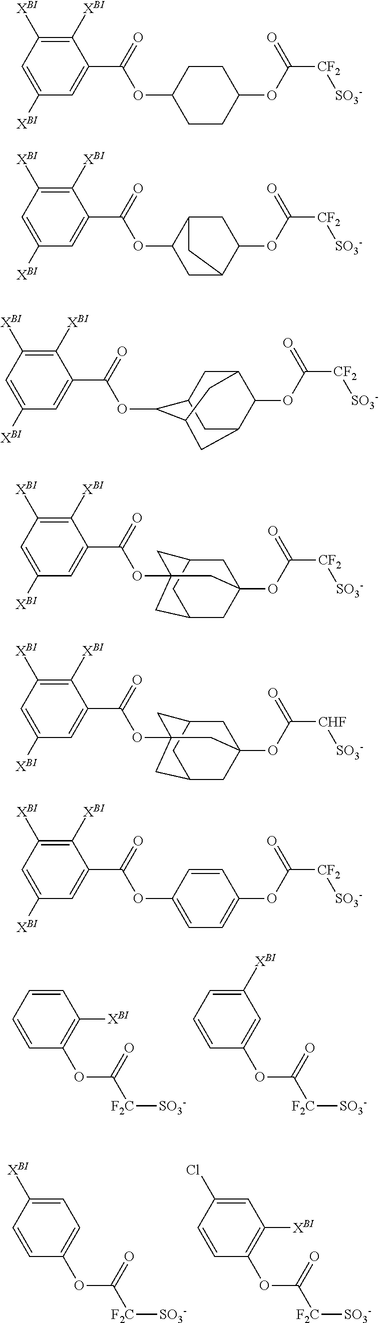

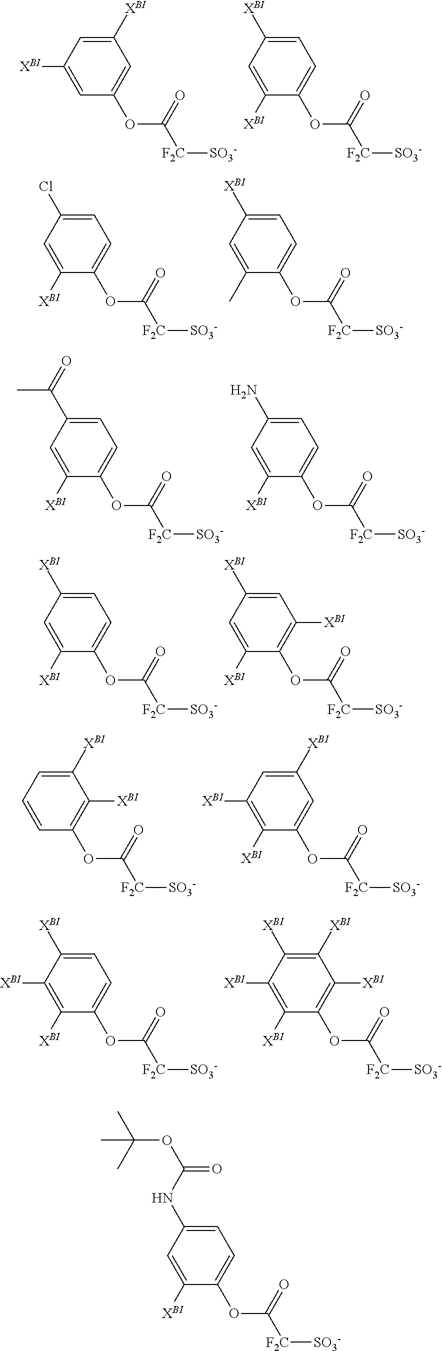

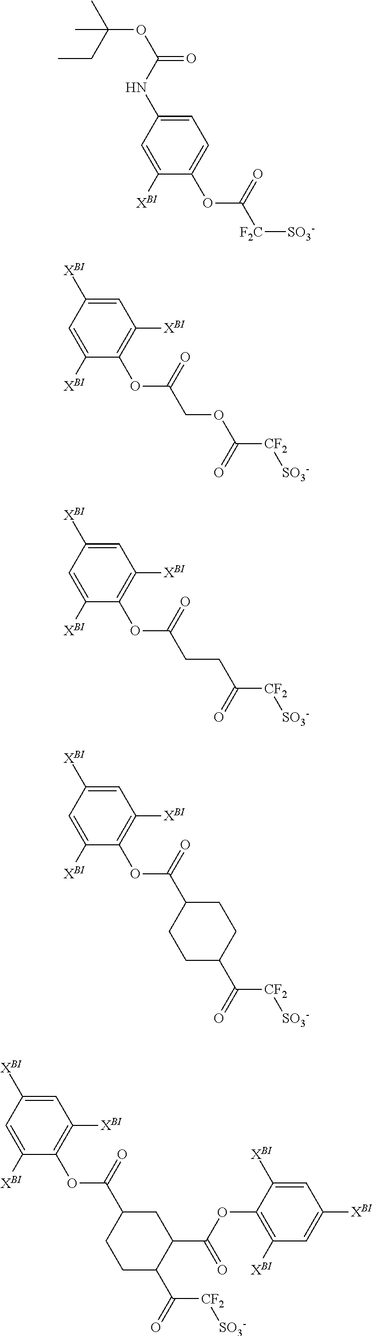

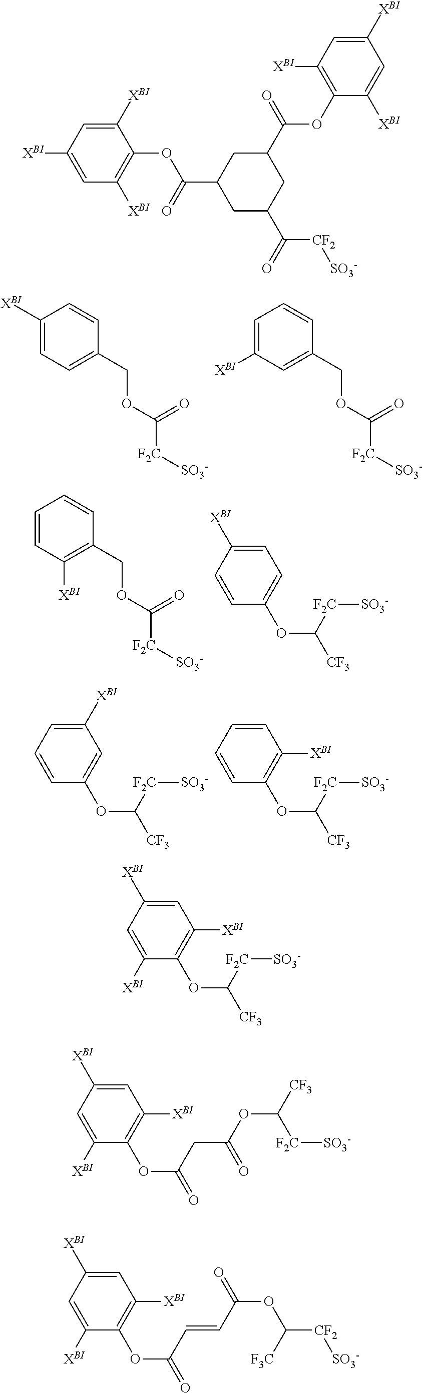

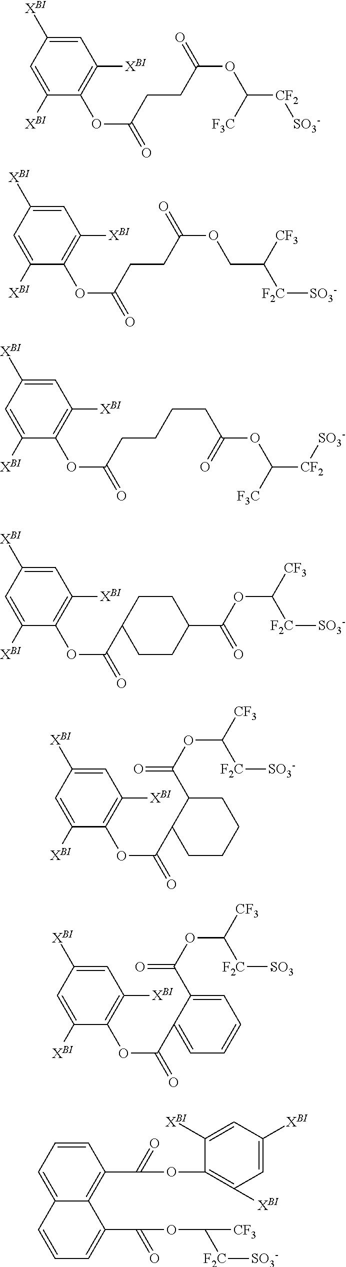

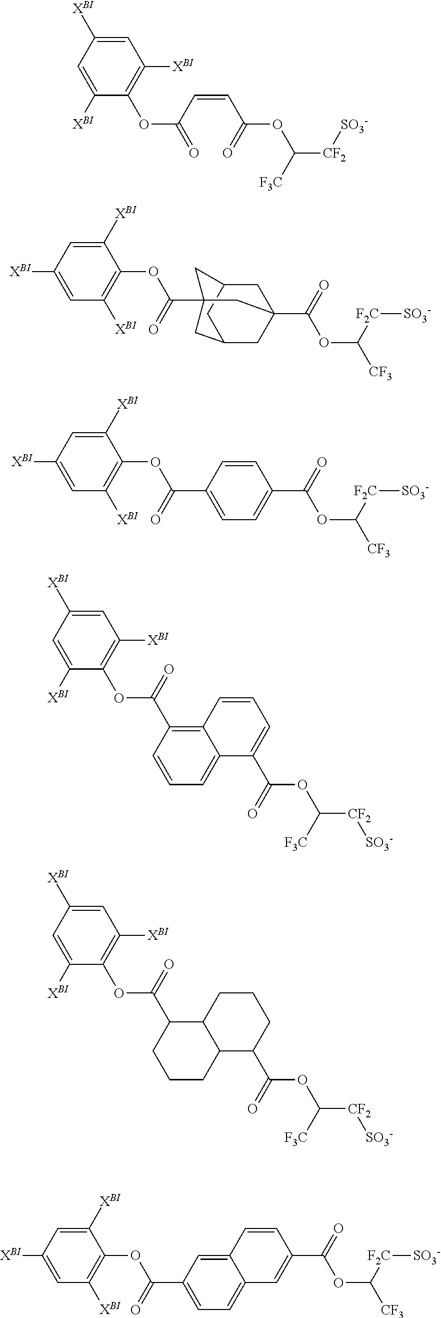

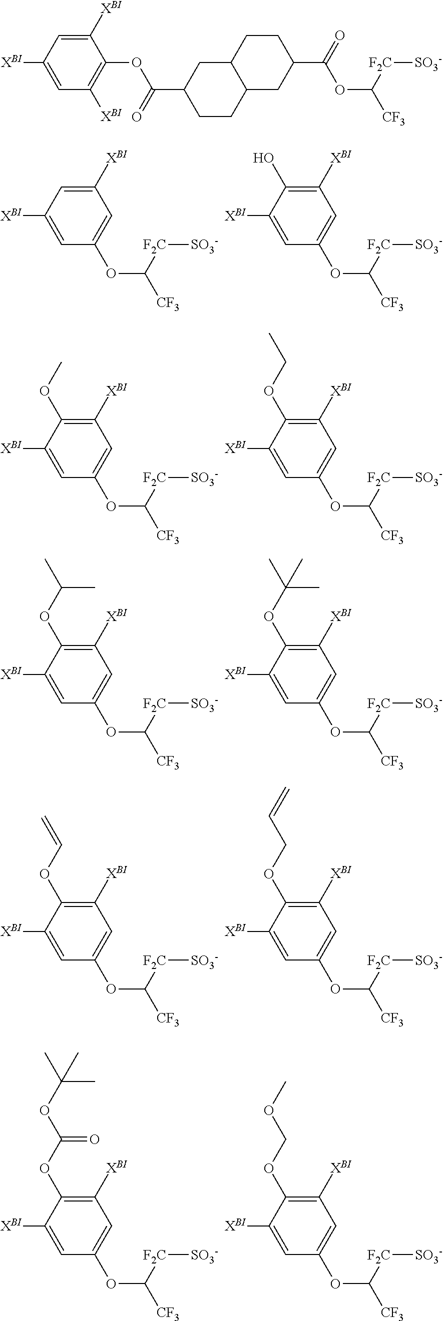

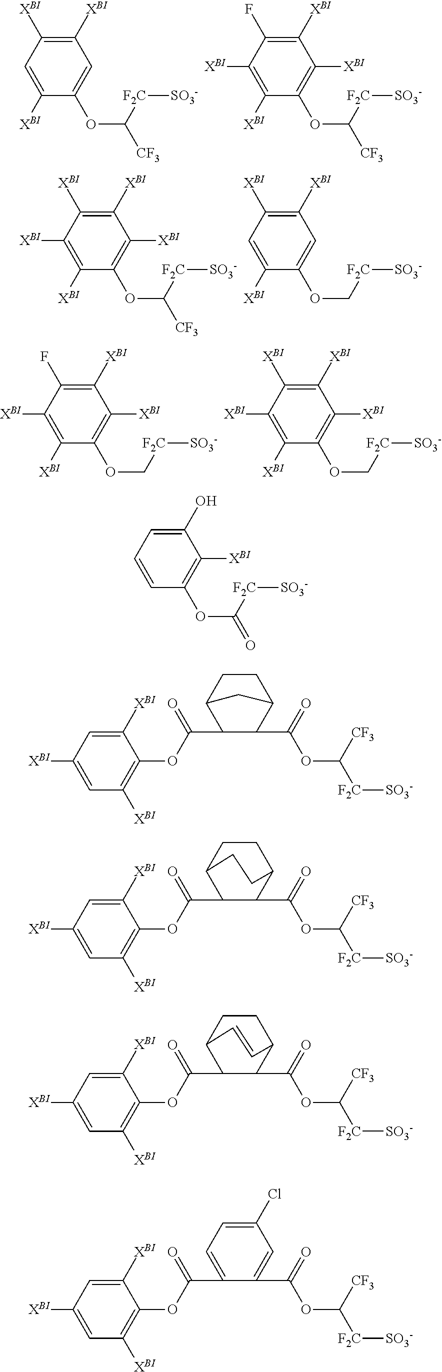

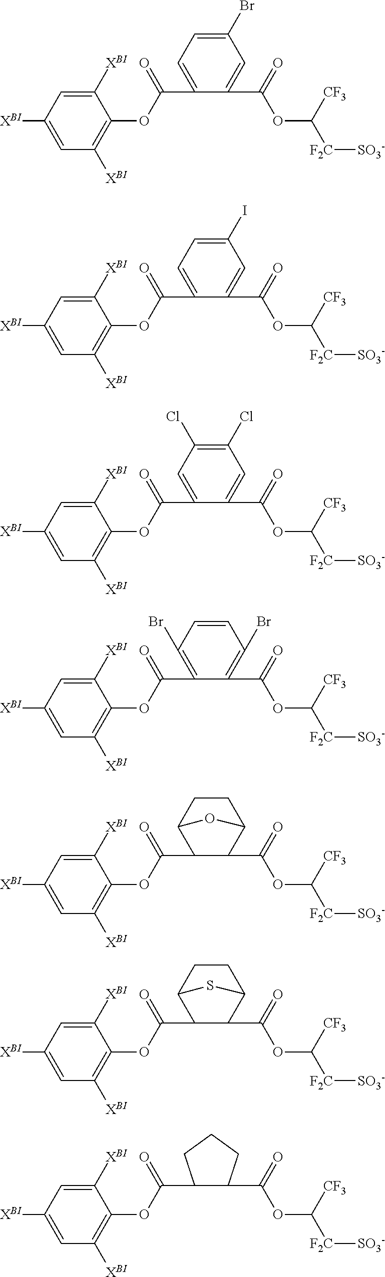

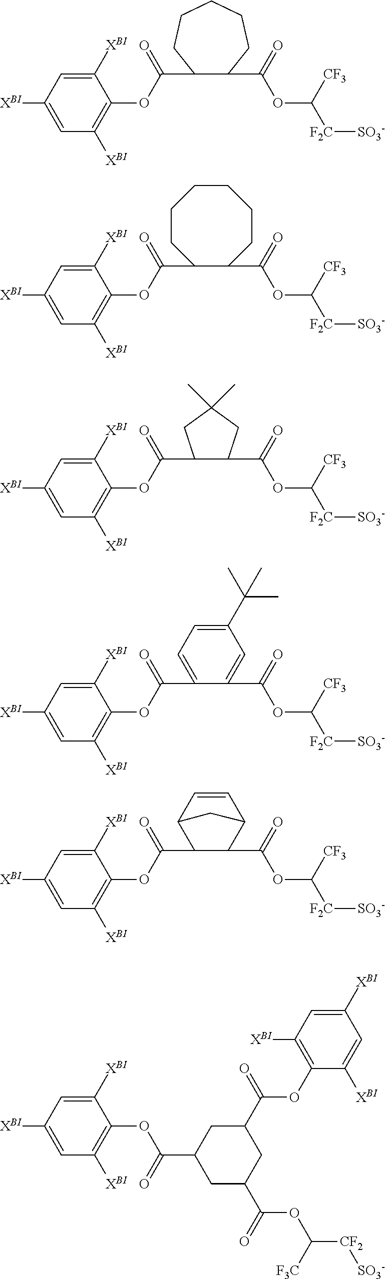

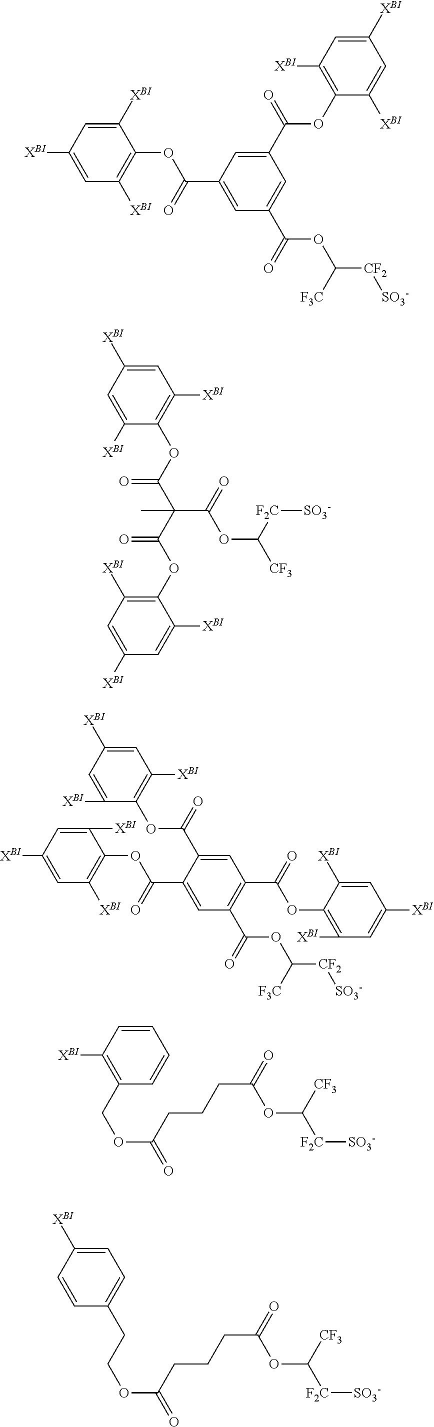

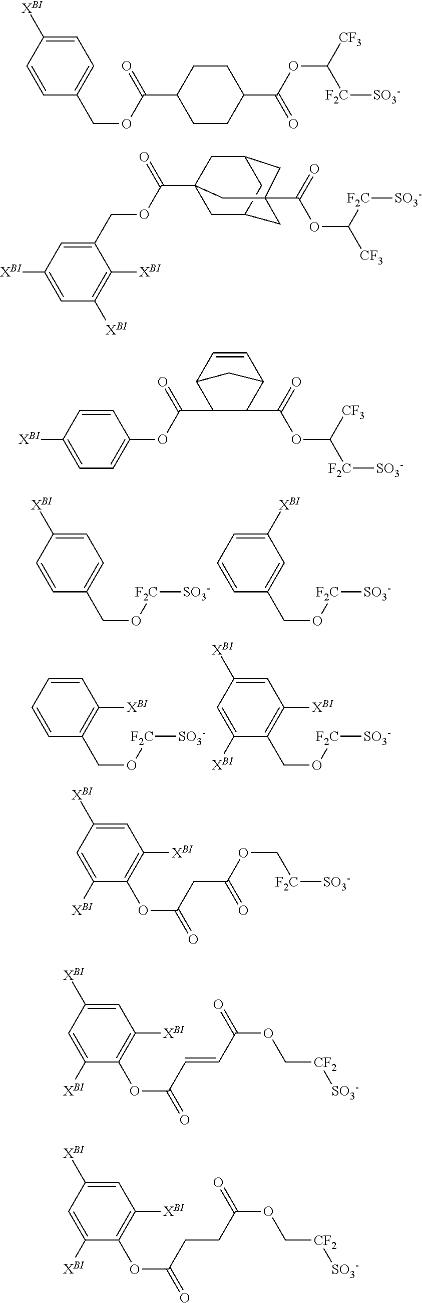

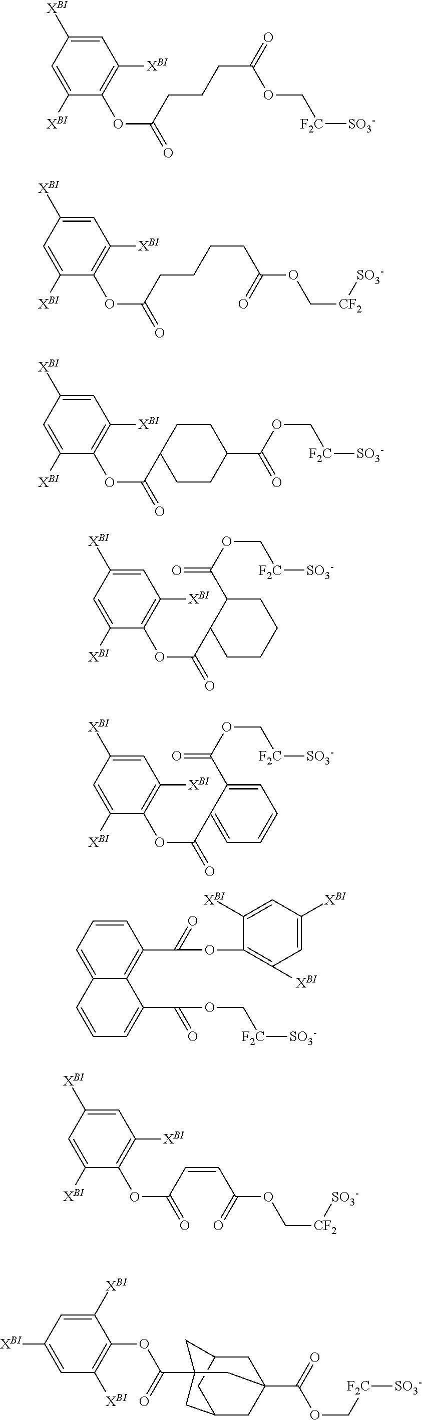

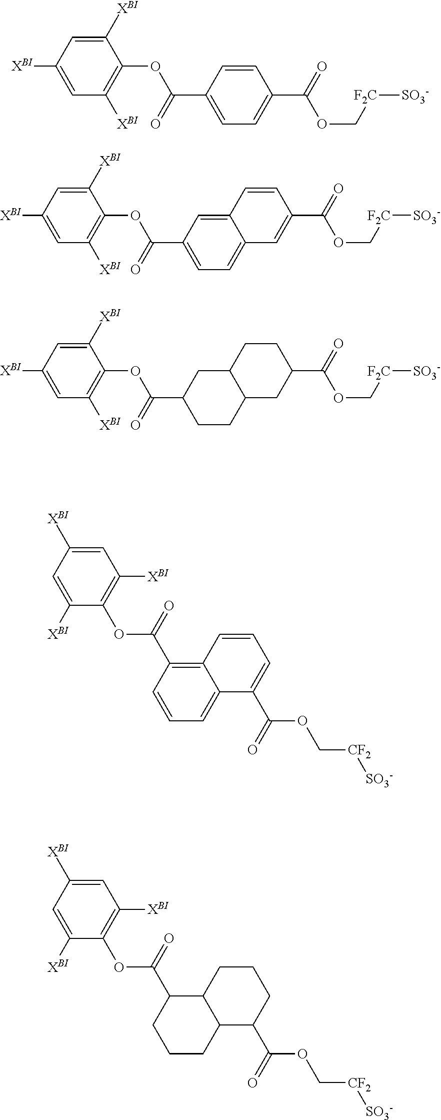

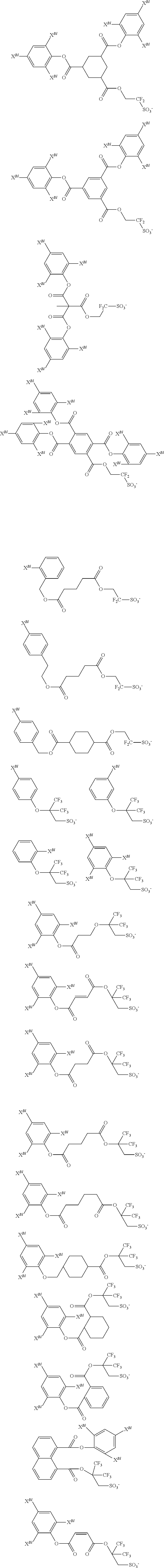

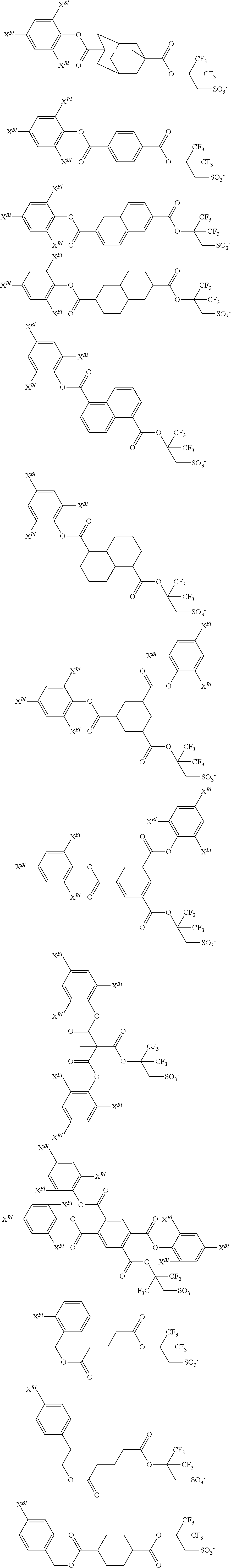

As the anion of the strong acid, a fluorobenzenesulfonate anion bonded to an aromatic group containing an iodine atom as described in JP6648726B2, an anion having a mechanism of being decomposed by an acid as described in WO2021/200056A1 and JP2021-070692A, an anion having a cyclic ether group as described in JP2018180525A and JP2021-035935A, and an anion as described in JP2018-092159A can also be used.

Further, as the anion of the strong acid, an anion of a bulky benzenesulfonate derivative not containing a fluorine atom as described in JP2006-276759A, JP2015-117200A, JP2016-065016A, and JP2019-202974A, and a benzenesulfonate anion and an alkylsulfonate anion not containing a fluorine atom bonded to an aromatic group containing an iodine atom as described in JP6645464B2 can also be used.

Further, as the anion of the strong acid, an anion of bis-sulfonic acid as described in JP2015-206932A, an anion of sulfonamide or sulfonimide having a sulfonic acid on one side and a different one on the other side as described in WO2020/158366A1, and an anion having a sulfonic acid on one side and a carboxylic acid on the other side as described in JP2015-024989A can also be used.

Furthermore, as one of the other photo-acid generators for component (D), one represented by the following general formula (5) is also preferable.

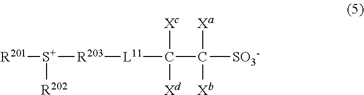

In the general formula (5), R²⁰¹ and R²⁰² each independently represent a hydrocarbyl group having 1 to 30 carbon atoms and optionally containing a heteroatom. R²⁰³ represents a hydrocarbylene group having 1 to 30 carbon atoms and optionally containing a heteroatom. Furthermore, any two of R²⁰¹, R²⁰², and R²⁰³ may be bonded to each other to form a ring together with the sulfur atom to which they are bonded.

The hydrocarbyl group having 1 to 30 carbon atoms represented by R²⁰¹ and R²⁰² may be saturated or unsaturated, and may be linear, branched, or cyclic. Specific examples thereof include alkyl groups having 1 to 30 carbon atoms, such as methyl, ethyl, n-propyl, isopropyl, n-butyl, isobutyl, sec-butyl, tert-butyl, tert-pentyl, n-pentyl, n-hexyl, n-octyl, 2-ethylhexyl, n-nonyl, and n-decyl groups; cyclic saturated hydrocarbyl groups having 3 to 30 carbon atoms, such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}]decyl, and adamantyl groups; aryl groups having 6 to 30 carbon atoms, such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl, and anthracenyl groups; and groups obtained by combining these groups. Furthermore, some or all of the hydrogen atoms of the hydrocarbyl groups may be substituted with a group containing a heteroatom such as an oxygen atom, a sulfur atom, a nitrogen atom, or a halogen atom, and some of the —CH₂— of the hydrocarbyl groups may be substituted with a group containing a heteroatom such as an oxygen atom, a sulfur atom, or a nitrogen atom, and as a result, they may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group.

The hydrocarbylene group having 1 to 30 carbon atoms represented by R²⁰³ may be saturated or unsaturated, and may be linear, branched, or cyclic. Specific examples thereof include alkanediyl groups having 1 to 30 carbon atoms, such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl groups; cyclic saturated hydrocarbylene groups having 3 to 30 carbon atoms, such as cyclopentanediyl, cyclohexanediyl, norbornanediyl, and adamantanediyl groups; and arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene, and tert-butylnaphthylene groups. Furthermore, some or all of the hydrogen atoms of the hydrocarbylene group may be substituted with a group containing a heteroatom such as an oxygen atom, a sulfur atom, a nitrogen atom, or a halogen atom, and some of the —CH₂— of the hydrocarbylene group may be substituted with a group containing a heteroatom such as an oxygen atom, a sulfur atom, or a nitrogen atom, and as a result, it may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group. The heteroatom is preferably an oxygen atom.

In the general formula (5), L¹¹ represents a hydrocarbylene group having 1 to 20 carbon atoms and optionally containing a single bond, an ether bond, or a heteroatom. The hydrocarbylene group may be saturated or unsaturated, and may be linear, branched, or cyclic. Specific examples thereof include the same ones as those given as examples of the hydrocarbylene group represented by R²⁰³.

In the general formula (5), X^{a}, X^{b}, X^{c}, and X^{d} each independently represent a hydrogen atom, a fluorine atom, or a trifluoromethyl group. However, at least one of X^{a}, X^{b}, X^{c}, and X^{d} represents a fluorine atom or a trifluoromethyl group.

As the photo-acid generator represented by the general formula (5), one represented by the following general formula (5′) is preferable.

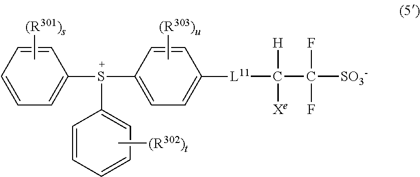

In the general formula (5′), L¹¹ is as defined above. X^{e} represents a hydrogen atom or a trifluoromethyl group, and preferably a trifluoromethyl group. R³⁰¹, R³⁰², and R³⁰³ each independently represent a hydrogen atom, or a hydrocarbyl group having 1 to 20 carbon atoms and optionally containing a heteroatom. The hydrocarbyl group may be saturated or unsaturated, and may be linear, branched, or cyclic. Specific examples thereof include the same ones as those given as examples of the hydrocarbyl group represented by R^{fa1} in the general formula (Xa-1-1). “s” and “t” each independently represent an integer from 0 to 5, and “u” represents an integer from 0 to 4.

Examples of the photo-acid generator represented by the general formula (5) include the same ones as those given as examples of the photo-acid generator represented by formula (2) in JP2017-026980A.

Among the other photo-acid generators, those containing an anion represented by the general formula (Xa-1-1) or (Xa-4) are particularly preferable because they have low acid diffusion and excellent solubility in solvents. Moreover, one represented by the general formula (5′) is particularly preferable because it has extremely low acid diffusion.

When the chemically amplified resist composition of the present invention contains the other photo-acid generators (D), the content thereof is preferably 0.1 to 40 parts by mass, and more preferably 0.5 to 20 parts by mass, per 80 parts by mass of the base polymer (A). When the amount of the other photo-acid generators (D) added is within the above range, the resolution is good and there is no risk of problems with foreign matter occurring after development or during stripping of the resist film, so it is preferable. The other photo-acid generators (D) may be used alone or in a combination of two or more types.

### [Surfactant (E)]

The chemically amplified resist composition of the present invention may further contain a surfactant as component (E). The surfactant (E) is preferably a surfactant that is insoluble or poorly soluble in water and soluble in an alkaline developer, or a surfactant that is insoluble or poorly soluble in both water and an alkaline developer. For such surfactants, reference can be made to those described in JP2010-215608A and JP2011-016746A.

Among the surfactants described in the above publications, preferred surfactants that are insoluble or poorly soluble in water and alkaline developers include FC-4430 (manufactured by 3M Company), Surflon (registered trademark) S-381 (manufactured by AGC Seimi Chemical Co., Ltd.), Olfine (registered trademark) E1004 (manufactured by Nissin Chemical Industry Co., Ltd.), KH-20, KH-30 (manufactured by AGC Seimi Chemical Co., Ltd.), and oxetane ring-opening polymers represented by the following general formula (surf-1).

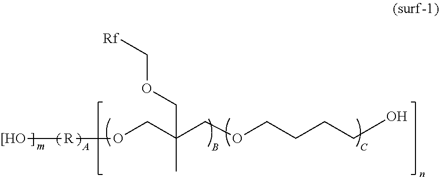

Here, R, Rf, A, B, C, “m”, and “n” apply only to the general formula (surf-1), regardless of the above descriptions. R represents a divalent to tetravalent aliphatic group having 2 to 5 carbon atoms. Examples of the aliphatic group include divalent groups such as an ethylene group, a 1,4-butylene group, a 1,2-propylene group, a 2,2-dimethyl-1,3-propylene group, and a 1,5-pentylene group, and examples of trivalent or tetravalent groups include the following.

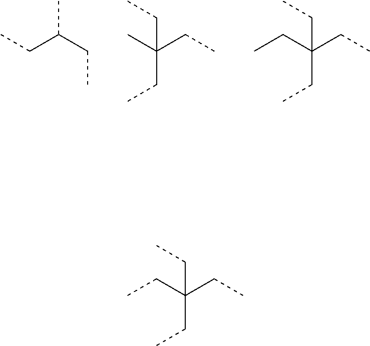

In the formulae, broken lines represent attachment points, and each formula are partial structures derived from glycerol, trimethylolethane, trimethylolpropane, and pentaerythritol, respectively.

Among these, 1,4-butylene group, 2,2-dimethyl-1,3-propylene group, etc. are preferable.

Rf represents a trifluoromethyl group or a pentafluoroethyl group, and preferably a trifluoromethyl group. “m” represents an integer from 0 to 3, “n” represents an integer from 1 to 4, and the sum of “n” and “m” is the valence of R and is an integer from 2 to 4. A represents 1. B represents an integer from 2 to 25, and is preferably an integer from 4 to 20. C represents an integer from 0 to 10, and is preferably 0 or 1. Furthermore, the order of the constituent units in the general formula (surf-1) is not specified, and they may be bonded in blocks or randomly. The manufacture of partially fluorinated oxetane ring-opening polymer surfactants is described in detail in U.S. Pat. No. 5,650,483A.

Surfactants that are insoluble or poorly soluble in water and soluble in alkaline developers have the function of reducing water penetration and leaching by orienting themselves on the surface of the resist film when a resist protective film is not used in ArF immersion lithography. Therefore, they are useful for suppressing the elution of water-soluble components from the resist film and reducing damage to exposure equipment, and are also useful because they become soluble during alkaline aqueous solution development after exposure or post-exposure bake (PEB), making them less likely to become foreign matter that causes defects. Such surfactants are insoluble or poorly soluble in water and soluble in an alkaline developer, and are polymeric surfactants that are also called hydrophobic resins; in particular, those that have high water repellency and improve water slippage are preferable.

Specific examples of such polymeric surfactants include those containing at least one repeating unit selected from those represented by any of the following general formulae (6A) to (6E).

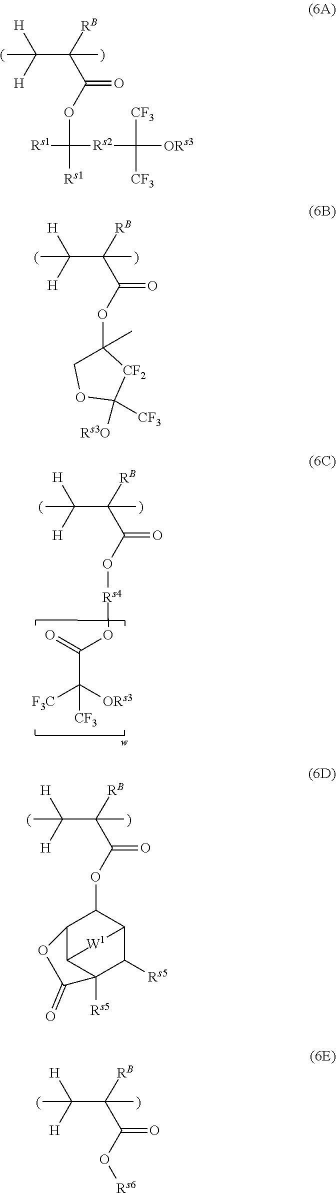

In the general formulae (6A) to (6E), R^{B} is a hydrogen atom, a fluorine atom, a methyl group, or a trifluoromethyl group. W¹ represents —CH₂—, —CH₂CH₂—, —O—, or two separated —H. R^{s1} each independently represents a hydrogen atom, or a hydrocarbyl group having 1 to 10 carbon atoms. R^{s2} represents a single bond, or a linear or branched hydrocarbylene group having 1 to 5 carbon atoms. R^{s3} each independently represents a hydrogen atom, a hydrocarbyl group having 1 to 15 carbon atoms, a fluorinated hydrocarbyl group, or an acid-labile group. When R^{s3} represents a hydrocarbyl group or a fluorinated hydrocarbyl group, an ether bond or a carbonyl group may be present between the carbon-carbon bonds. R^{s4} represents a (w+1)-valent hydrocarbon group or fluorinated hydrocarbon group having 1 to 20 carbon atoms. “w” represents 1, 2, or 3. R^{s5} each independently represent a hydrogen atom, or a group represented by —C(═O)—O—R^{sa}. R^{sa} represents a fluorinated hydrocarbyl group having 1 to 20 carbon atoms. R^{s6} represents a hydrocarbyl group or a fluorinated hydrocarbyl group having 1 to 15 carbon atoms, and an ether bond or a carbonyl group may be present between the carbon-carbon bonds.

The hydrocarbyl group having 1 to 10 carbon atoms represented by R^{s1} preferably represents a saturated hydrocarbyl group, and may be linear, branched, or cyclic. Specific examples thereof include alkyl groups having 1 to 10 carbon atoms, such as methyl, ethyl, n-propyl, isopropyl, n-butyl, isobutyl, sec-butyl, tert-butyl, n-pentyl, n-hexyl, n-heptyl, n-octyl, n-nonyl, and n-decyl groups; and cyclic saturated hydrocarbyl groups having 3 to 10 carbon atoms, such as cyclopropyl, cyclobutyl, cyclopentyl, cyclohexyl, adamantyl, and norbornyl groups. Of these, those having 1 to 6 carbon atoms are preferable.

The hydrocarbylene group represented by R^{s2} is preferably a saturated hydrocarbylene group, and may be linear, branched, or cyclic. Specific examples include a methylene group, an ethylene group, a propylene group, a butylene group, and a pentylene group.

The hydrocarbyl group represented by R^{s3} or R^{s6} may be saturated or unsaturated, and may be linear, branched, or cyclic. Specific examples thereof include saturated hydrocarbyl groups and aliphatic unsaturated hydrocarbyl groups such as alkenyl groups and alkynyl groups, with saturated hydrocarbyl groups being preferable. Examples of the saturated hydrocarbyl group include, in addition to those given as examples of the hydrocarbyl groups represented by R^{s1}, an undecyl group, a dodecyl group, a tridecyl group, a tetradecyl group, and a pentadecyl group. Examples of the fluorinated hydrocarbyl group represented by R^{s3} or R^{s6} include groups in which some or all of the hydrogen atoms bonded to carbon atoms of the aforementioned hydrocarbyl groups have been substituted with fluorine atoms. As mentioned above, an ether bond or a carbonyl group may be present between these carbon-carbon bonds.

Specific examples of the acid-labile group represented by R^{s3} include groups represented by the general formulae (AL-1) to (AL-3), trialkylsilyl groups in which each alkyl group has 1 to 6 carbon atoms, and oxo-group-containing alkyl groups having 4 to 20 carbon atoms.

The (w+1)-valent hydrocarbon group or fluorinated hydrocarbon group represented by R^{s4} may be linear, branched, or cyclic, and specific examples thereof include groups obtained by further eliminating “w” hydrogen atoms from the above-mentioned hydrocarbyl groups or fluorinated hydrocarbyl groups.

The fluorinated hydrocarbyl group represented by R^{sa} is preferably saturated and may be linear, branched, or cyclic. Specific examples thereof include those in which some or all of the hydrogen atoms of the hydrocarbyl groups have been substituted with fluorine atoms, and specific examples thereof include a trifluoromethyl group, a 2,2,2-trifluoroethyl group, a 3,3,3-trifluoro-1-propyl group, a 3,3,3-trifluoro-2-propyl group, a 2,2,3,3-tetrafluoropropyl group, a 1,1,1,3,3,3-hexafluoroisopropyl group, a 2,2,3,3,4,4,4-heptafluorobutyl group, a 2,2,3,3,4,4,5,5-octafluoropentyl group, a 2,2,3,3,4,4,5,5,6,6,7,7-dodecafluoroheptyl group, a 2-(perfluorobutyl)ethyl group, a 2-(perfluorohexyl)ethyl group, a 2-(perfluorooctyl)ethyl group, and a 2-(perfluorodecyl)ethyl group.

Specific examples of the repeating unit represented by any one of the general formulae (6A) to (6E) include, but are not limited to, the following. In the following formulae, R^{B} is as defined above.

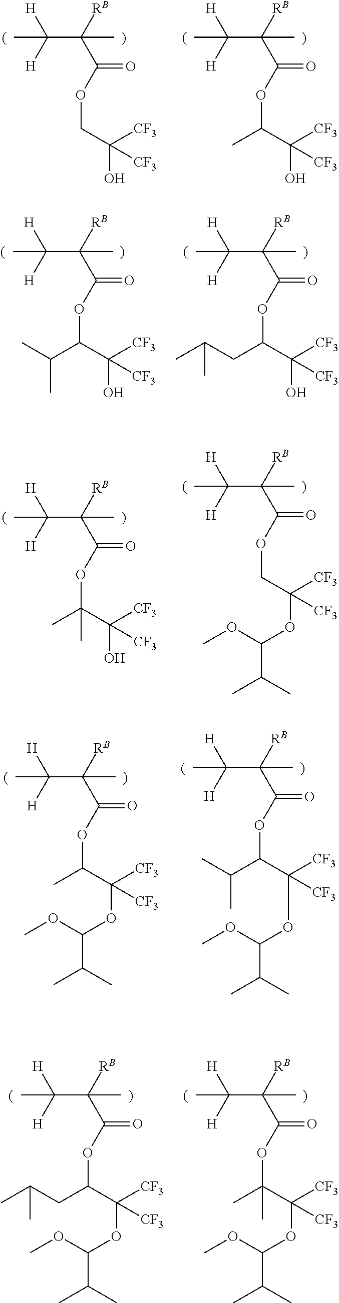

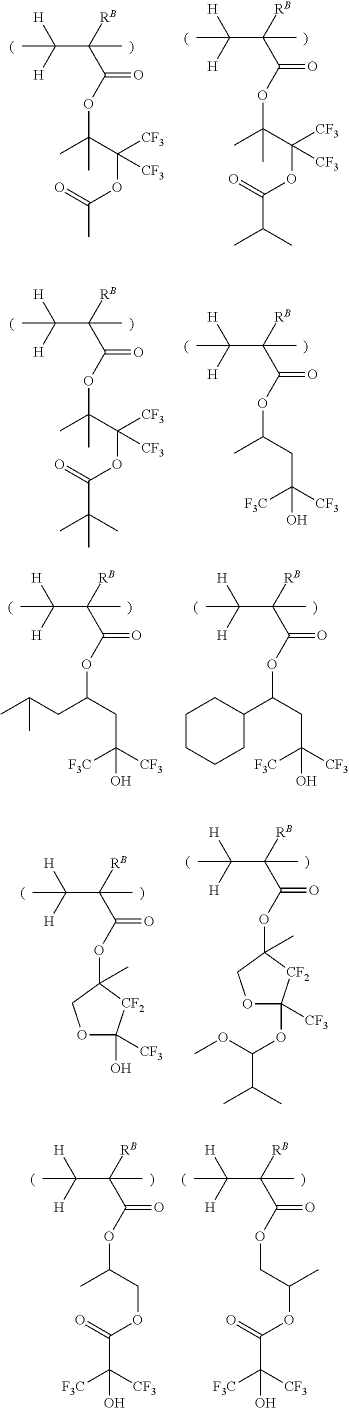

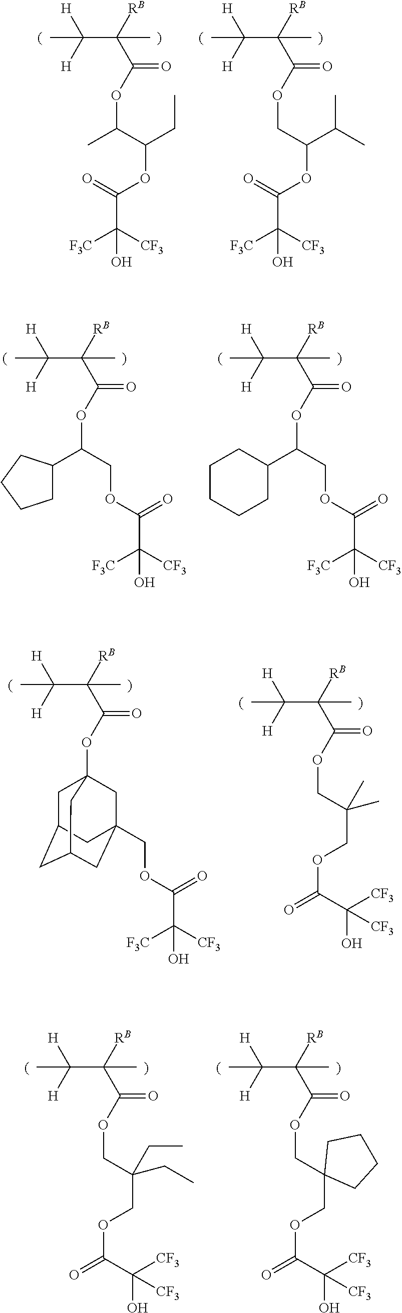

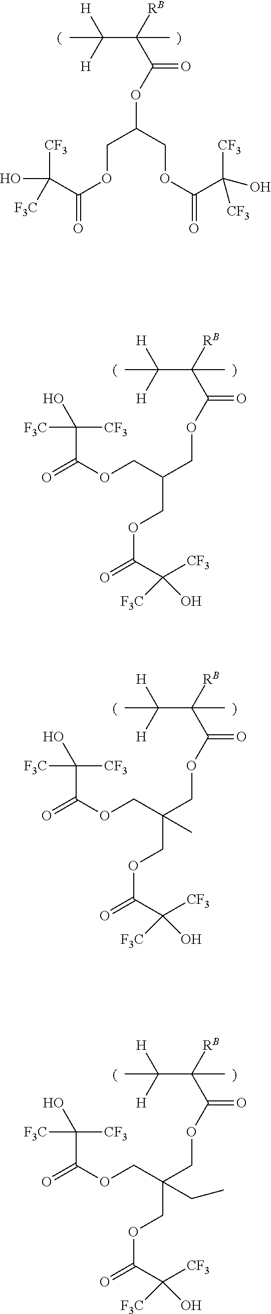

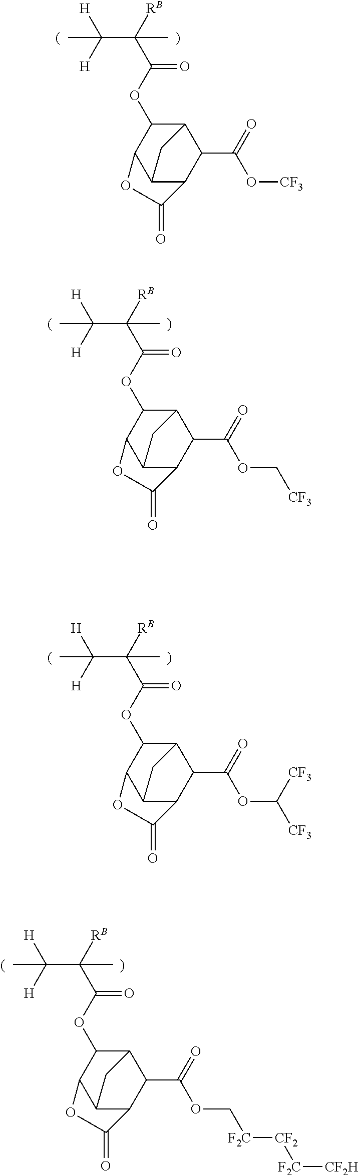

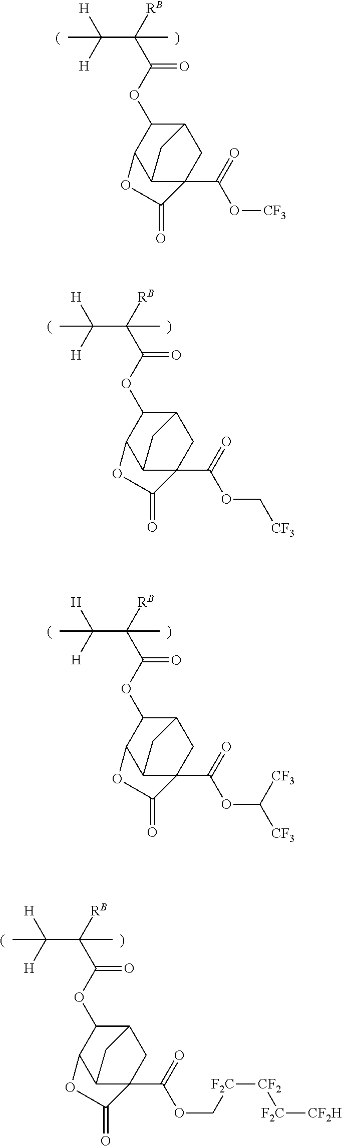

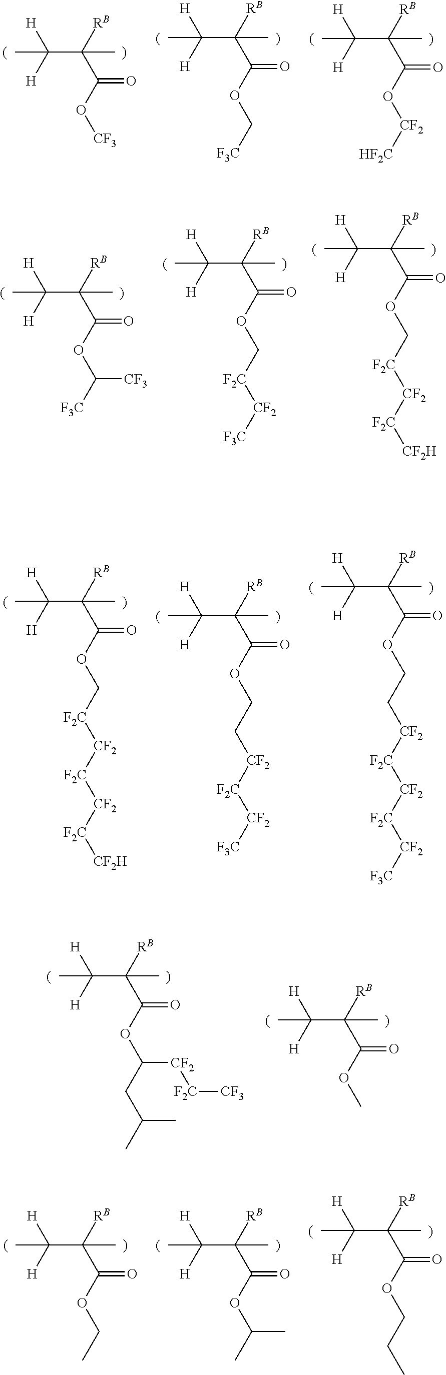

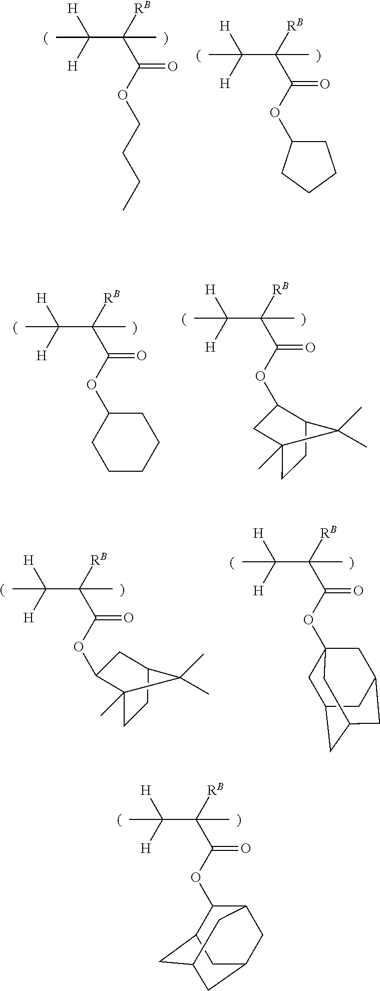

The polymeric surfactant may further contain other repeating units other than the repeating units represented by the general formulae (6A) to (6E). Examples of other repeating units include those obtained from methacrylic acid and α-trifluoromethylacrylic acid derivatives. In the polymeric surfactant, the content of the repeating units represented by the general formulae (6A) to (6E) is preferably 20 mol % or more, more preferably 60 mol % or more, and further preferably 100 mol %, of all repeating units.

The Mw of the polymeric surfactant is preferably 1,000 to 500,000, and more preferably 3,000 to 100,000. The Mw/Mn is preferably 1.0 to 2.0, and more preferably 1.0 to 1.6.

The polymeric surfactant can be synthesized by heating a monomer containing an unsaturated bond that gives the repeating units represented by the general formulae (6A) to (6E) and, if necessary, other repeating units in an organic solvent in the presence of a radical initiator to polymerize the monomer. Examples of the organic solvent used during polymerization include toluene, benzene, THF, diethyl ether, and dioxane. Examples of polymerization initiators include AIBN, 2,2′-azobis(2,4-dimethylvaleronitrile), dimethyl 2,2-azobis(2-methylpropionate), benzoyl peroxide, and lauroyl peroxide. The reaction temperature is preferably 50 to 100° C. The reaction time is preferably 4 to 24 hours. The acid-labile group may be used as is after being introduced into the monomer, and may be protected or partially protected after polymerization.

When synthesizing the polymeric surfactant, a known chain transfer agent such as dodecyl mercaptan and 2-mercaptoethanol may be used to adjust the molecular weight. In this case, the amount of these chain transfer agents added is preferably 0.01 to 10 mol % based on the total number of moles of the monomers to be polymerized.

When the chemically amplified resist composition of the present invention contains the surfactant (E), the content thereof is preferably 0.1 to 50 parts by mass, and more preferably 0.5 to 10 parts by mass, per 80 parts by mass of the base polymer (A). When the content of the surfactant (E) is 0.1 parts by mass or more, the receding contact angle between the resist film surface and water is sufficiently improved, and when it is 50 parts by mass or less, the dissolution rate of the resist film surface in the developer is low, and the height of the formed fine pattern is sufficiently maintained. The surfactant (E) may be used alone or in a combination of two or more types.

### [Other Components (F)]

The chemically amplified resist composition of the present invention may contain, as other components (F), a compound that decomposes when exposed to acid to generate acid (acid-proliferating compound), an organic acid derivative, a fluorine-substituted alcohol, a compound with Mw of 3,000 or less whose solubility in a developer changes due to the action of an acid (dissolution inhibitor), etc. For the acid-proliferating compound, reference can be made to the compounds described in JP2009-269953A and JP2010-215608A. When the acid-proliferating compound is included, the content thereof is preferably 0 to 5 parts by mass, and more preferably 0 to 3 parts by mass, per 80 parts by mass of the base polymer (A). When the content is within this range, it is possible to control acid diffusion, and the degradation of resolution and pattern shape is unlikely to occur. For the organic acid derivative, fluorine-substituted alcohol, and dissolution inhibitor, reference can be made to the compounds described in JP2009-269953A and JP2010-215608A.

### [Patterning Process]

The present invention provides a patterning process, containing the steps of forming a resist film on a substrate using the chemically amplified resist composition described above, exposing the resist film with a high-energy beam, and developing the exposed resist film using a developer.

The substrate used can be, for example, a substrate for manufacturing integrated circuits (Si, SiO₂, SiN, SiON, TiN, WSi, BPSG, SOG, organic antireflective film, etc.) or a substrate for mask circuit manufacturing (Cr, CrO, CrON, MoSi₂, SiO₂, etc.).

The resist film can be formed, for example, by applying the chemically amplified resist composition onto a substrate by a method such as spin coating so that the film thickness is preferably 0.05 to 2 μm, and then pre-baking the applied composition on a hot plate preferably at 60 to 150° C. for 1 to 10 minutes, and more preferably at 80 to 140° C. for 1 to 5 minutes.

Examples of high-energy beams used to expose resist films include KrF excimer laser light, ArF excimer laser light, an electron beam (EB), and extreme ultraviolet (EUV) with a wavelength of 3 to 15 nm. When KrF excimer laser light, ArF excimer laser light, or EUV is used for exposure, a mask for forming a desired pattern is used and irradiation is performed so that the exposure amount is preferably 1 to 200 mJ/cm², and more preferably 10 to 100 mJ/cm². When EB is used, irradiation is performed using a mask for forming a desired pattern or directly, and the exposure amount is preferably 1 to 300 μC/cm², and more preferably 10 to 200 μC/cm².

In addition to the normal exposure method, the immersion method can also be used, in which a liquid with a refractive index of 1.0 or more is placed between the resist film and the projection lens. In that case, it is also possible to use a water-insoluble protective film.

The water-insoluble protective film is used to prevent substances from eluting from the resist film and to increase the water slippage of the film surface, and are broadly divided into two types. One is an organic solvent stripping type, which needs to be stripped using an organic solvent that does not dissolve the resist film before developing with an alkaline aqueous solution, and the other is an alkaline aqueous solution soluble type, which is soluble in alkaline developer and removes the protective film along with removing the soluble parts of the resist film. The latter is particularly preferred as a material based on a polymer having a 1,1,1,3,3,3-hexafluoro-2-propanol residue, which is insoluble in water and soluble in an alkaline developer, and which is dissolved in an alcohol solvent having 4 or more carbon atoms, an ether solvent having 8 to 12 carbon atoms, and a mixed solvent of these. The above-mentioned water-insoluble, alkaline-developer-soluble surfactant can also be dissolved in an alcohol solvent having 4 or more carbon atoms, an ether solvent having 8 to 12 carbon atoms, or a mixed solvent of these to produce a material.

After exposure, PEB may be performed. PEB can be performed, for example, by heating on a hot plate, preferably at 60 to 150° C. for 1 to 5 minutes, and more preferably at 80 to 140° C. for 1 to 3 minutes.

The development is carried out, for example, using a developer of an alkaline aqueous solution such as tetramethylammonium hydroxide (TMAH) of preferably 0.1 to 5% by mass, and more preferably 2 to 3% by mass, and by a conventional method such as a dip method, a puddle method, or a spray method for preferably 0.1 to 3 minutes, and more preferably 0.5 to 2 minutes, so that the exposed area dissolves and the desired pattern is formed on the substrate.

After the resist film is formed, pure water rinsing may be performed to extract the acid generator, etc. from the film surface or to wash away particles, and rinsing may be performed to remove water remaining on the film after exposure.

Furthermore, patterns may be formed by a double patterning method. Double patterning methods include the trench method, in which the base of a 1:3 trench pattern is processed by the first exposure and etching, then a 1:3 trench pattern is formed by shifting the position and performing the second exposure to form a 1:1 pattern, and the line method, in which the first base of a 1:3 isolated pattern is processed by the first exposure and etching, then the second base is processed by shifting the position and performing the second exposure to form a 1:3 isolated pattern under the first base, forming a 1:1 pattern with half the pitch.

In the patterning process of the present invention, a negative tone development method may be used in which an organic solvent is used as a developer instead of the alkaline aqueous solution to dissolve the unexposed areas.

Examples of developers that can be used to perform the negative tone development include 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, and 2-phenylethyl acetate. These organic solvents may be used alone or in a combination of two or more types.

### EXAMPLE

Hereinafter, the present invention will be described more specifically with reference to Examples and Comparative Examples. However, the present invention is not limited thereto. The equipment used is as follows.

MALDI TOF-MS: S3000 manufactured by JEOL Ltd.

### [1] Synthesis of Onium Salt Type Monomers

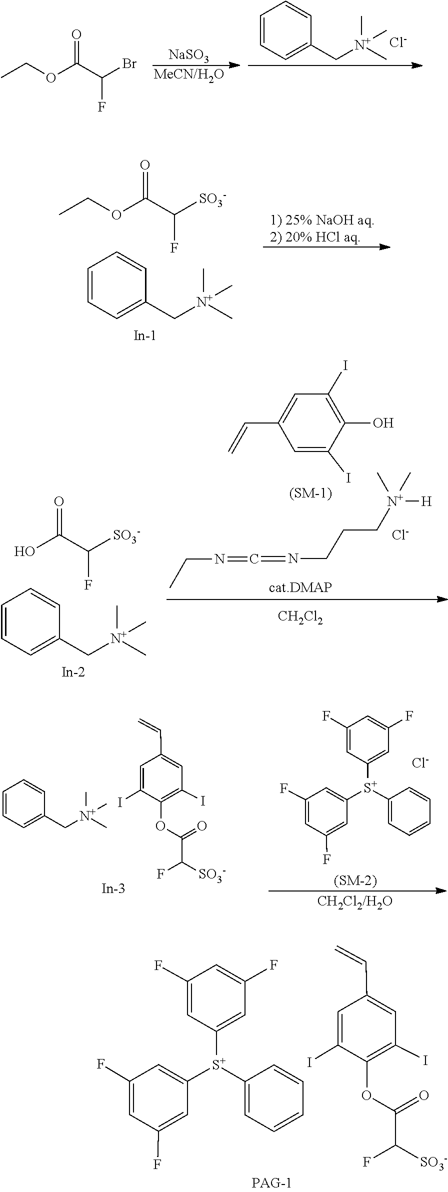

### [Example 1-1] Synthesis of Onium Salt Type Monomer PAG-1

### (1) Synthesis of Intermediate In-1

Under a nitrogen atmosphere, ethyl bromofluoroacetate (30.0 g), sodium sulfite (20.4 g), acetonitrile (120 g), and water (60 g) were added and stirred at 60° C. for 5 hours. The reaction solution was transferred to a separatory funnel, and the aqueous layer was washed twice with hexane (50 g). Benzyltrimethylammonium chloride (30.1 g) and methylene chloride (150 g) were added to the obtained aqueous solution, and it was stirred at room temperature for 1 hour. The reaction solution was transferred to a separatory funnel, and the organic layer was washed once with water (50 g). After isolating the organic layer, the solvent was concentrated to obtain 42.4 g of the intermediate In-1 as an oil (yield: 78%).

### (2) Synthesis of Intermediate In-2

Under a nitrogen atmosphere, the intermediate In-1 (42.4 g) was dissolved in a 25% by mass aqueous solution of sodium hydroxide (22.2 g) and stirred at 40° C. for 6 hours. Then, 20% by mass hydrochloric acid (26.5 g) was added to neutralize the reaction solution. Then, acetonitrile (100 g) was added and azeotropic dehydration was performed to obtain 31.8 g of the intermediate In-2 as an oil (yield 82%).

### (3) Synthesis of Intermediate In-3

Under a nitrogen atmosphere, intermediate In-2 (30.7 g), raw material SM-1 (37.2 g), 4-dimethylaminopyridine (DMAP) (1.2 g), and methylene chloride (200 g) were prepared and cooled in an ice bath. While maintaining the temperature inside the reaction vessel at 20° C. or less, 1-ethyl-3-(3-dimethylaminopropyl)carbodiimide hydrochloride (23.0 g) was added as a powder. After the addition, the temperature was raised to room temperature and the mixture was aged for 8 hours. After aging, water was added to stop the reaction, the reaction was subjected to normal aqueous work-up, and the solvent was then distilled off to obtain the intermediate In-3 as an oil (yield: 60.8 g, 92%).

### (4) Synthesis of Monomer PAG-1

Under a nitrogen atmosphere, intermediate In-3 (60.8 g), raw material SM-2 (37.5 g), methylene chloride (300 g), and water (150 g) were prepared and stirred at room temperature for 30 minutes. The organic layer was isolated, washed with water, and then concentrated under reduced pressure. The residue was washed with diisopropyl ether and concentrated to obtain 74.0 g of the onium salt type monomer PAG-1 as an oil (yield: 95%).

The TOF-MS results for PAG-1 are shown below.

MALDI TOF-MS: POSITIVE M⁺ 335 (C₁₈H₁₁F₄S⁺-equivalent)

NEGATIVE M⁻ 511 (C₁₀H₆FI₂O₅S⁻-equivalent)

### [Examples 1-2 to 1-9] Synthesis of Onium Salt Type Monomers PAG-2 to PAG-9

The onium salt type monomers PAG-2 to PAG-9 shown below were synthesized using the corresponding raw materials and various organic synthesis reactions.

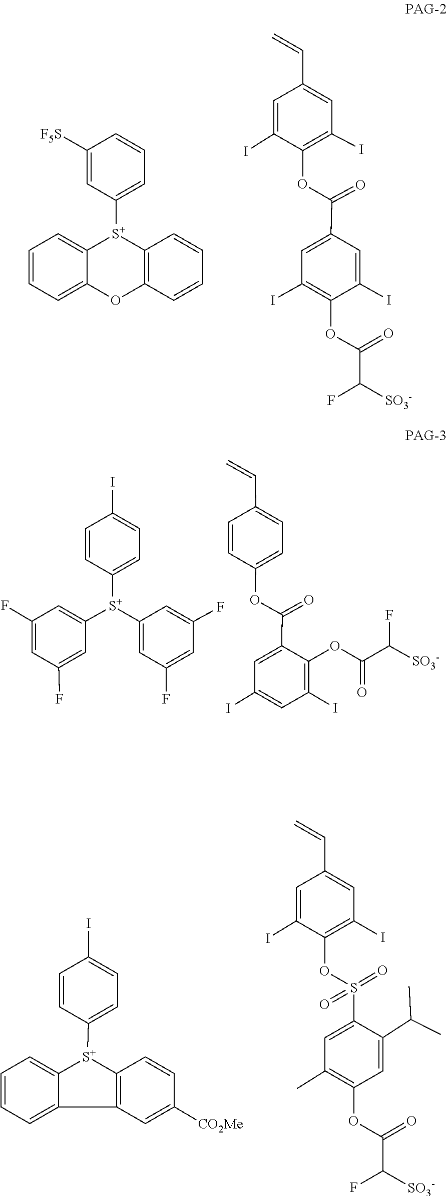

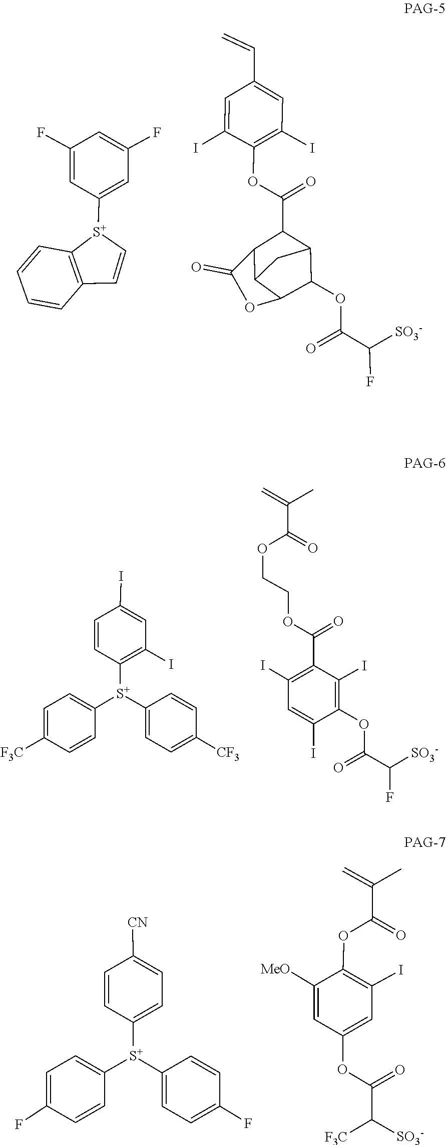

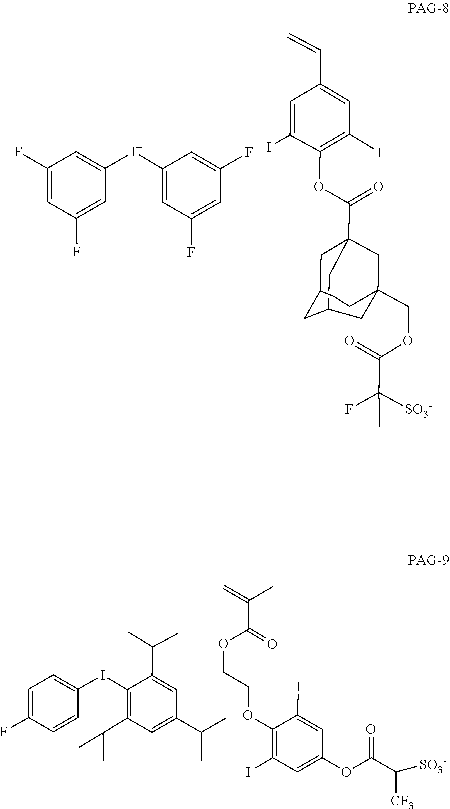

### [Comparative Examples 1-1 to 1-6] Synthesis of Comparative Onium Salt Type Monomers PAG-A to PAG-F

The comparative onium salt type monomers PAG-A to PAG-F shown below were synthesized using the corresponding raw materials and various organic synthesis reactions.

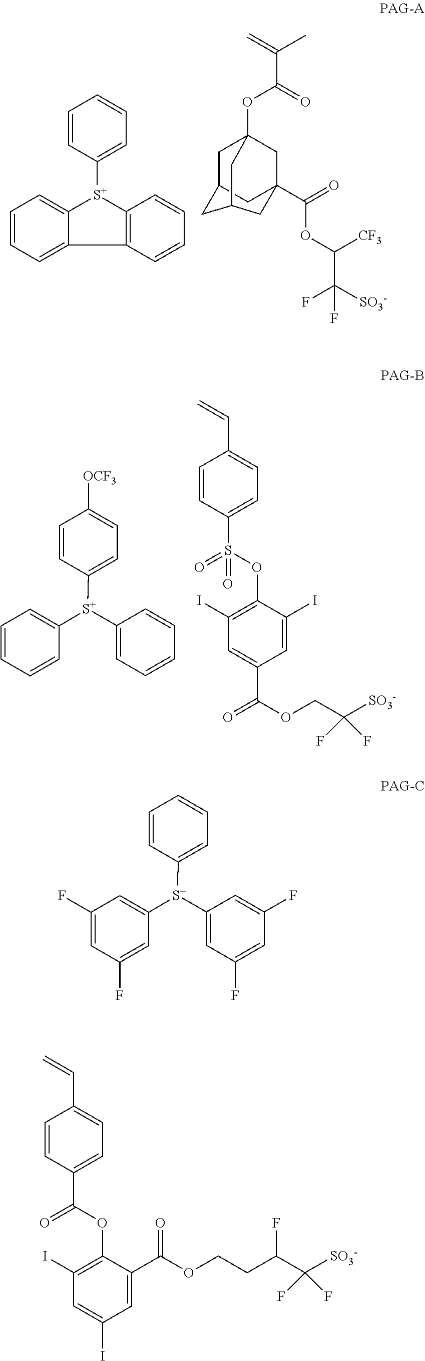

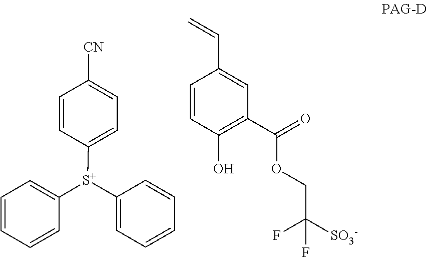

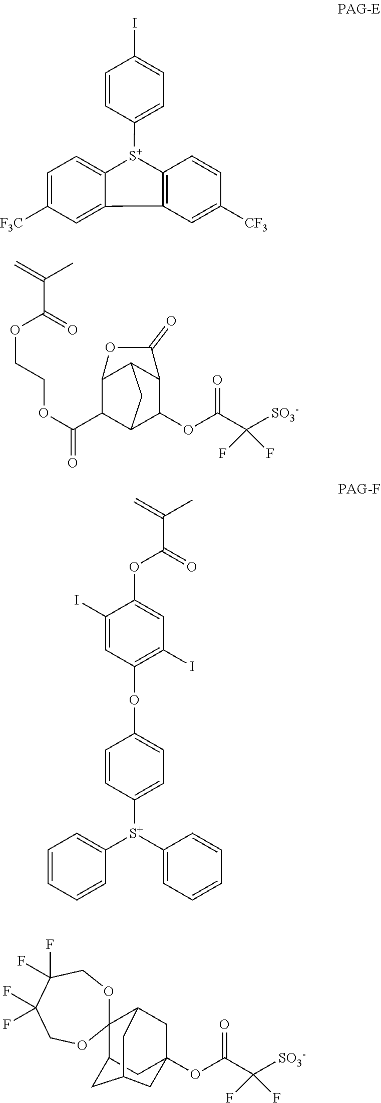

### [2] Synthesis of Base Polymer

Among the monomers used in the synthesis of the base polymer, those other than PAG-1 to PAG-9 and PAG-A to PAG-F are as follows.

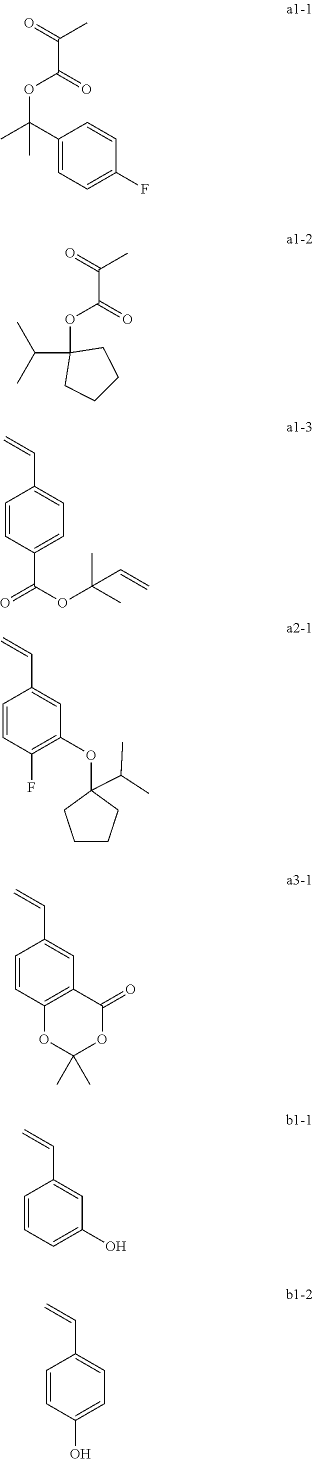

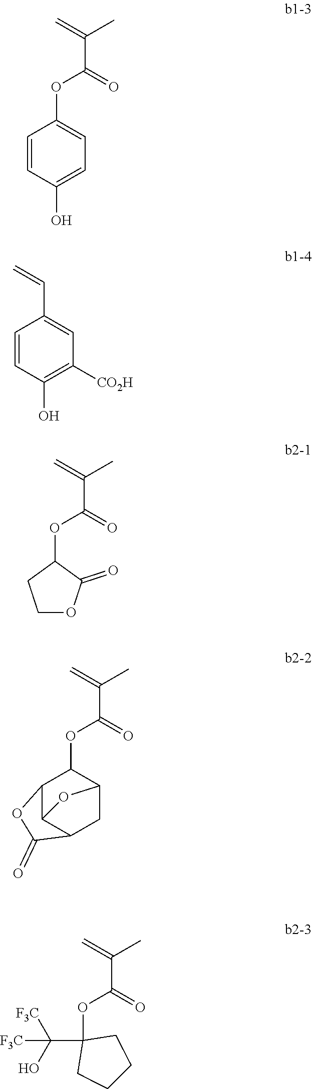 [SMILES: C=C(C)C(=O)Oc1ccc(O)cc1 ; C=Cc1ccc(O)c(C(=O)O)c1 ; C=C(C)C(=O)OC1CCOC1=O ; C=C(C)C(=O)OC1C2CC3C(=O)OC1C3O2 ; C=C(C)C(=O)OC1(C(O)(C(F)(F)F)C(F)(F)F)CCCC1]

### [Example 2-1] Synthesis of Polymer P-1

Under a nitrogen atmosphere, monomer a1-1 (43.0 g), monomer b1-1 (12.6 g), monomer PAG-1 (44.5 g), 4.04 g of V-601 (manufactured by Wako Pure Chemical Industries, Ltd.), and 140 g of MEK were placed in a flask to prepare a monomer-polymerization initiator solution. MEK (46 g) was placed in a separate flask under a nitrogen atmosphere and heated to 80° C. while stirring, after which the monomer-polymerization initiator solution was added dropwise over a period of 4 hours. After the dropwise addition was completed, the polymerization solution was stirred for 2 hours while maintaining the temperature at 80° C., and then cooled to room temperature. The obtained polymerization liquid was added dropwise to 2,000 g of vigorously stirred hexane, and the precipitated polymer was separated by filtration. The obtained polymer was further washed twice with 600 g of hexane and then vacuum dried at 50° C. for 20 hours to obtain polymer P-1 in the form of a white powder (yield: 98.1 g, 98%). The Mw of polymer P-1 was 9,200 and the Mw/Mn was 1.63. The Mw is a polystyrene-equivalent measurement value obtained by GPC using DMF as a solvent.

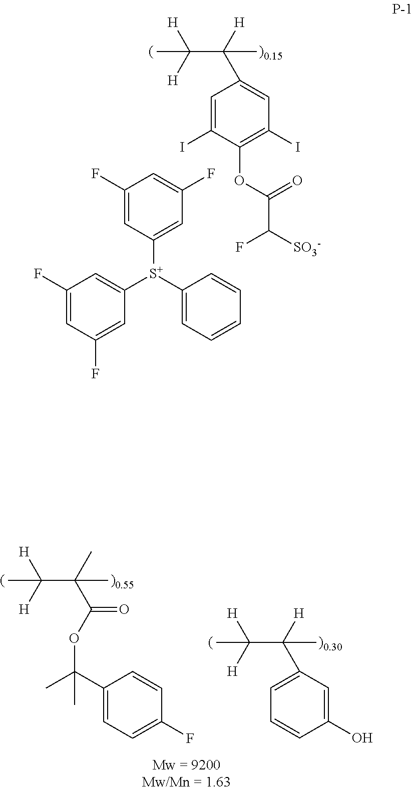

### [Examples 2-2 to 2-26, Comparative Examples 2-1 to 2-18] Synthesis of Polymers P-2 to P-26 and CP-1 to CP-18

The polymers shown in Tables 1 and 2 were manufactured in the same manner as in Example 2-1, except that the types and compounding ratios of each monomer were changed.

TABLE 1

| | | Introduction | | Introduction | | Introduction | | Introduction | | Introduction | | |
|---|---|---|---|---|---|---|---|---|---|---|---|---|
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| P-1 | PAG-1 | 15 | a1-1 | 55 | b1-1 | 30 | — | — | — | — | 9200 | 1.63 |
| P-2 | PAG-2 | 15 | a1-1 | 55 | b1-1 | 30 | — | — | — | — | 9200 | 1.62 |
| P-3 | PAG-3 | 15 | a1-1 | 55 | b1-1 | 30 | — | — | — | — | 9400 | 1.63 |
| P-4 | PAG-4 | 15 | a1-1 | 55 | b1-1 | 30 | — | — | — | — | 9400 | 1.63 |
| P-5 | PAG-5 | 15 | a1-1 | 55 | b1-1 | 30 | — | — | — | — | 9200 | 1.61 |
| P-6 | PAG-6 | 15 | a1-1 | 55 | b1-1 | 30 | — | — | — | — | 9300 | 1.62 |
| P-7 | PAG-7 | 15 | a1-1 | 55 | b1-1 | 30 | — | — | — | — | 9200 | 1.65 |
| P-8 | PAG-8 | 15 | a1-1 | 55 | b1-1 | 30 | — | — | — | — | 9300 | 1.61 |
| P-9 | PAG-9 | 15 | a1-1 | 55 | b1-1 | 30 | — | — | — | — | 9600 | 1.62 |
| P-10 | PAG-1 | 15 | a1-2 | 55 | b1-1 | 30 | — | — | — | — | 9400 | 1.60 |
| P-11 | PAG-1 | 15 | a1-3 | 55 | b1-1 | 30 | — | — | — | — | 9200 | 1.61 |
| P-12 | PAG-1 | 15 | a2-1 | 55 | b1-1 | 30 | — | — | — | — | 9800 | 1.62 |
| P-13 | PAG-1 | 15 | a3-1 | 45 | b1-1 | 40 | — | — | — | — | 8900 | 1.63 |
| P-14 | PAG-2 | 15 | a1-1 | 55 | b1-2 | 30 | — | — | — | — | 9300 | 1.61 |
| P-15 | PAG-2 | 15 | a1-1 | 55 | b1-3 | 30 | — | — | — | — | 9100 | 1.62 |
| P-16 | PAG-2 | 15 | a1-1 | 55 | b1-4 | 30 | — | — | — | — | 9300 | 1.64 |
| P-17 | PAG-1 | 15 | a1-1 | 30 | a2-1 | 20 | b1-1 | 35 | — | — | 9000 | 1.62 |
| P-18 | PAG-3 | 15 | a1-1 | 35 | a3-1 | 15 | b1-2 | 35 | — | — | 9600 | 1.61 |
| P-19 | PAG-4 | 15 | a1-2 | 30 | a2-1 | 15 | b1-3 | 40 | — | — | 9500 | 1.62 |
| P-20 | PAG-6 | 10 | a1-1 | 35 | a2-1 | 15 | b1-1 | 30 | b2-1 | 10 | 9400 | 1.63 |
| P-21 | PAG-7 | 15 | a1-2 | 35 | a3-1 | 15 | b1-2 | 25 | b2-2 | 10 | 9200 | 1.64 |
| P-22 | PAG-9 | 15 | a1-1 | 50 | b1-1 | 30 | b2-3 | 5 | — | — | 8900 | 1.61 |
| P-23 | PAG-1 | 5 | a1-1 | 55 | b1-2 | 40 | — | — | — | — | 9500 | 1.62 |
| P-24 | PAG-2 | 5 | a1-1 | 30 | a1-3 | 25 | b1-2 | 40 | — | — | 9400 | 1.63 |
| P-25 | PAG-3 | 5 | a1-2 | 30 | a2-1 | 20 | b1-4 | 35 | b2-1 | 10 | 9200 | 1.61 |
| P-26 | PAG-6 | 5 | a1-2 | 30 | a2-1 | 20 | b1-4 | 35 | b2-1 | 10 | 9400 | 1.62 |

TABLE 2

| | | Introduction | | Introduction | | Introduction | | Introduction | | Introduction | | |
|---|---|---|---|---|---|---|---|---|---|---|---|---|
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| CP-1 | PAG-A | 15 | a1-1 | 55 | b1-1 | 30 | — | — | — | — | 9500 | 1.62 |
| CP-2 | PAG-B | 15 | a1-1 | 55 | b1-1 | 30 | — | — | — | — | 9100 | 1.61 |
| CP-3 | PAG-C | 15 | a1-1 | 55 | b1-1 | 30 | — | — | — | — | 9300 | 1.63 |
| CP-4 | PAG-D | 15 | a1-1 | 55 | b1-1 | 30 | — | — | — | — | 9100 | 1.62 |
| CP-5 | PAG-E | 15 | a1-1 | 55 | b1-1 | 30 | — | — | — | — | 9000 | 1.65 |
| CP-6 | PAG-F | 15 | a1-1 | 55 | b1-1 | 30 | — | — | — | — | 9500 | 1.62 |
| CP-7 | PAG-B | 15 | a1-2 | 55 | b1-1 | 30 | — | — | — | — | 9600 | 1.61 |
| CP-8 | PAG-C | 15 | a3-1 | 45 | b1-1 | 40 | — | — | — | — | 9700 | 1.63 |
| CP-9 | PAG-D | 15 | a1-1 | 55 | b1-3 | 30 | — | — | — | — | 9500 | 1.63 |
| CP-10 | PAG-E | 15 | a1-1 | 55 | b1-4 | 30 | — | — | — | — | 9400 | 1.62 |
| CP-11 | PAG-B | 15 | a1-1 | 35 | a3-1 | 15 | b1-2 | 35 | — | — | 9700 | 1.64 |
| CP-12 | PAG-D | 10 | a1-1 | 35 | a2-1 | 15 | b1-1 | 30 | b2-1 | 10 | 9500 | 1.62 |
| CP-13 | PAG-C | 15 | a1-2 | 35 | a3-1 | 15 | b1-2 | 25 | b2-2 | 10 | 9300 | 1.61 |
| CP-14 | PAG-F | 15 | a1-1 | 50 | b1-1 | 30 | b2-3 | 5 | — | — | 9100 | 1.60 |
| CP-15 | PAG-A | 5 | a1-1 | 55 | b1-2 | 40 | — | — | — | — | 9300 | 1.62 |
| CP-16 | PAG-C | 5 | a1-2 | 30 | a2-1 | 20 | b1-4 | 35 | b2-1 | 10 | 9400 | 1.63 |
| CP-17 | a1-1 | 60 | b1-1 | 40 | — | — | — | — | — | — | 5700 | 1.55 |
| CP-18 | a1-1 | 50 | b1-2 | 30 | b2-1 | 20 | — | — | — | — | 6100 | 1.54 |

### [3] Preparation of Chemically Amplified Resist Composition

### Examples 3-1 to 3-26, Comparative Examples 3-1 to 3-18

Base polymers (P-1 to P-26) containing onium salt type monomers (PAG-1 to PAG-9) of the present invention, base polymers (CP-1 to CP-18) containing comparative onium salt type monomers (PAG-A to PAG-F), photo-acid generators (PAG-X, PAG-Y), and quenchers (Q-1 to Q-4) were dissolved in a solvent containing 0.01% by mass of surfactant A (OMNOVA) in the compositions shown in Tables 3 and 4 below to prepare solutions, and the solutions were filtered through a 0.2 μm Teflon (registered trademark) type filter to prepare chemically amplified resist compositions (R-1 to R-26, CR-1 to CR-18).

TABLE 3

| | | | | Photo-acid | | | |
|---|---|---|---|---|---|---|---|
| | Resist | Base polymer | Quencher | generator | Solvent 1 | Solvent 2 | Solvent 3 |
| | composition | (Parts by mass) | (Parts by mass) | (Parts by mass) | (Parts by mass) | (Parts by mass) | (Parts by mass) |
| Example 3-1 | R-1 | P-1 (80) | Q-1 (8.0) | — | PGMEA (2250) | EL (2800) | DAA (550) |
| Example 3-2 | R-2 | P-2 (80) | Q-1 (8.2) | — | PGMEA (2250) | EL (2800) | DAA (550) |
| Example 3-3 | R-3 | P-3 (80) | Q-1 (8.0) | — | PGMEA (2250) | EL (2800) | DAA (550) |
| Example 3-4 | R-4 | P-4 (80) | Q-1 (7.8) | — | PGMEA (2250) | EL (2800) | DAA (550) |
| Example 3-5 | R-5 | P-5 (80) | Q-1 (8.0) | — | PGMEA (2250) | EL (2800) | DAA (550) |
| Example 3-6 | R-6 | P-6 (80) | Q-1 (8.0) | — | PGMEA (2250) | EL (2800) | DAA (550) |
| Example 3-7 | R-7 | P-7 (80) | Q-1 (8.2) | — | PGMEA (2250) | EL (2800) | DAA (550) |
| Example 3-8 | R-8 | P-8 (80) | Q-1 (8.0) | — | PGMEA (2250) | EL (2800) | DAA (550) |
| Example 3-9 | R-9 | P-9 (80) | Q-1 (7.6) | — | PGMEA (2250) | EL (2800) | DAA (550) |
| Example 3-10 | R-10 | P-10 (80) | Q-2 (8.2) | — | PGMEA (2250) | EL (2800) | DAA (550) |
| Example 3-11 | R-11 | P-11 (80) | Q-3 (8.0) | — | PGMEA (2250) | EL (2800) | DAA (550) |
| Example 3-12 | R-12 | P-12 (80) | Q-1 (8.0) | — | PGMEA (2250) | EL (2800) | DAA (550) |
| Example 3-13 | R-13 | P-13 (80) | Q-1 (8.2) | — | PGMEA (2250) | EL (2800) | DAA (550) |
| Example 3-14 | R-14 | P-14 (80) | Q-1 (8.0) | — | PGMEA (2250) | EL (2800) | DAA (550) |
| Example 3-15 | R-15 | P-15 (80) | Q-3 (7.8) | — | PGMEA (2250) | EL (2800) | DAA (550) |
| Example 3-16 | R-16 | P-16 (80) | Q-2 (8.0) | — | PGMEA (2250) | EL (2800) | DAA (550) |
| Example 3-17 | R-17 | P-17 (80) | Q-1 (8.0) | — | PGMEA (2250) | EL (2800) | DAA (550) |
| Example 3-18 | R-18 | P-18 (80) | Q-1 (7.8) | — | PGMEA (2250) | EL (2800) | DAA (550) |
| Example 3-19 | R-19 | P-19 (80) | Q-2 (8.0) | — | PGMEA (2250) | EL (2800) | DAA (550) |
| Example 3-20 | R-20 | P-20 (80) | Q-3 (7.8) | PAG-Y (15) | PGMEA (2250) | EL (2800) | DAA (550) |
| Example 3-21 | R-21 | P-21 (80) | Q-1 (4.0)/ | — | PGMEA (2250) | EL (2800) | DAA (550) |
| | | | Q-4 (3.8) | | | | |
| Example 3-22 | R-22 | P-22 (80) | Q-1 (8.2) | — | PGMEA (2250) | EL (2800) | DAA (550) |
| Example 3-23 | R-23 | P-23 (80) | Q-3 (7.6) | — | PGMEA (2250) | EL (2800) | DAA (550) |
| Example 3-24 | R-24 | P-24 (80) | Q-1 (8.0) | PAG-X (10) | PGMEA (2250) | EL (2800) | DAA (550) |
| Example 3-25 | R-25 | P-25 (80) | Q-2 (8.2) | PAG-X (10) | PGMEA (2250) | EL (2800) | DAA (550) |
| Example 3-26 | R-26 | P-26 (80) | Q-3 (8.0) | PAG-Y (15) | PGMEA (2250) | EL (2800) | DAA (550) |

TABLE 4

| | | | | Photo-acid | | | |
|---|---|---|---|---|---|---|---|
| | Resist | Base polymer | Quencher | generator | Solvent 1 | Solvent 2 | Solvent 3 |
| | composition | (Parts by mass) | (Parts by mass) | (Parts by mass) | (Parts by mass) | (Parts by mass) | (Parts by mass) |
| Comparative | CR-1 | CP-1 (80) | Q-1 (8.0) | — | PGMEA (2250) | EL (2800) | DAA (550) |
| Example 3-1 | | | | | | | |
| Comparative | CR-2 | CP-2 (80) | Q-1 (8.2) | — | PGMEA (2250) | EL (2800) | DAA (550) |
| Example 3-2 | | | | | | | |
| Comparative | CR-3 | CP-3 (80) | Q-1 (8.0) | — | PGMEA (2250) | EL (2800) | DAA (550) |
| Example 3-3 | | | | | | | |
| Comparative | CR-4 | CP-4 (80) | Q-1 (7.8) | — | PGMEA (2250) | EL (2800) | DAA (550) |
| Example 3-4 | | | | | | | |
| Comparative | CR-5 | CP-5 (80) | Q-1 (8.0) | — | PGMEA (2250) | EL (2800) | DAA (550) |
| Example 3-5 | | | | | | | |
| Comparative | CR-6 | CP-6 (80) | Q-1 (8.2) | | PGMEA (2250) | EL (2800) | DAA (550) |
| Example 3-6 | | | | | | | |
| Comparative | CR-7 | CP-7 (80) | Q-2 (8.2) | — | PGMEA (2250) | EL (2800) | DAA (550) |
| Example 3-7 | | | | | | | |
| Comparative | CR-8 | CP-8 (80) | Q-1 (8.2) | — | PGMEA (2250) | EL (2800) | DAA (550) |
| Example 3-8 | | | | | | | |
| Comparative | CR-9 | CP-9 (80) | Q-3 (7.8) | — | PGMEA (2250) | EL (2800) | DAA (550) |
| Example 3-9 | | | | | | | |
| Comparative | CR-10 | CP-10 (80) | Q-2 (8.0) | — | PGMEA (2250) | EL (2800) | DAA (550) |
| Example 3-10 | | | | | | | |
| Comparative | CR-11 | CP-11 (80) | Q-1 (8.0) | — | PGMEA (2250) | EL (2800) | DAA (550) |
| Example 3-11 | | | | | | | |
| Comparative | CR-12 | CP-12 (80) | Q-3 (7.8) | PAG-Y (15) | PGMEA (2250) | EL (2800) | DAA (550) |
| Example 3-12 | | | | | | | |
| Comparative | CR-13 | CP-13 (80) | Q-1 (4.0)/ | — | PGMEA (2250) | EL (2800) | DAA (550) |
| Example 3-13 | | | Q-4 (3.8) | | | | |
| Comparative | CR-14 | CP-14 (80) | Q-1 (8.2) | — | PGMEA (2250) | EL (2800) | DAA (550) |
| Example 3-14 | | | | | | | |
| Comparative | CR-15 | CP-15 (80) | Q-3 (7.6) | — | PGMEA (2250) | EL (2800) | DAA (550) |
| Example 3-15 | | | | | | | |
| Comparative | CR-16 | CP-16 (80) | Q-2 (8.2) | PAG-X (10) | PGMEA (2250) | EL (2800) | DAA (550) |
| Example 3-16 | | | | | | | |
| Comparative | CR-17 | CP-17 (80) | Q-1 (8.0) | PAG-X (24) | PGMEA (2250) | EL (2800) | DAA (550) |
| Example 3-17 | | | | | | | |
| Comparative | CR-18 | CP-18 (80) | Q-1 (8.0) | PAG-Y (24) | PGMEA (2250) | EL (2800) | DAA (550) |
| Example 3-18 | | | | | | | |

In Tables 3 and 4, the solvents, photo-acid generators PAG-X and PAG-Y, quenchers Q-1 to Q-4, and surfactant A are as follows.

Solvents: PGMEA (propylene glycol monomethyl ether acetate)
EL (Ethyl lactate)
DAA (Diacetone alcohol)

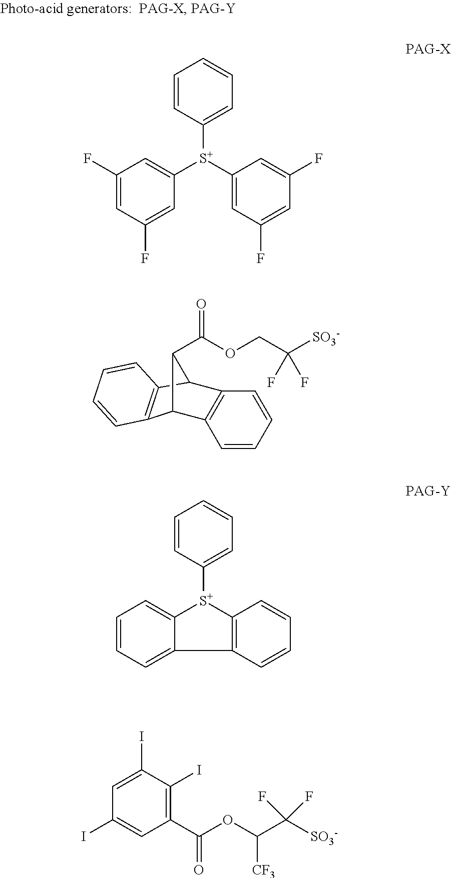

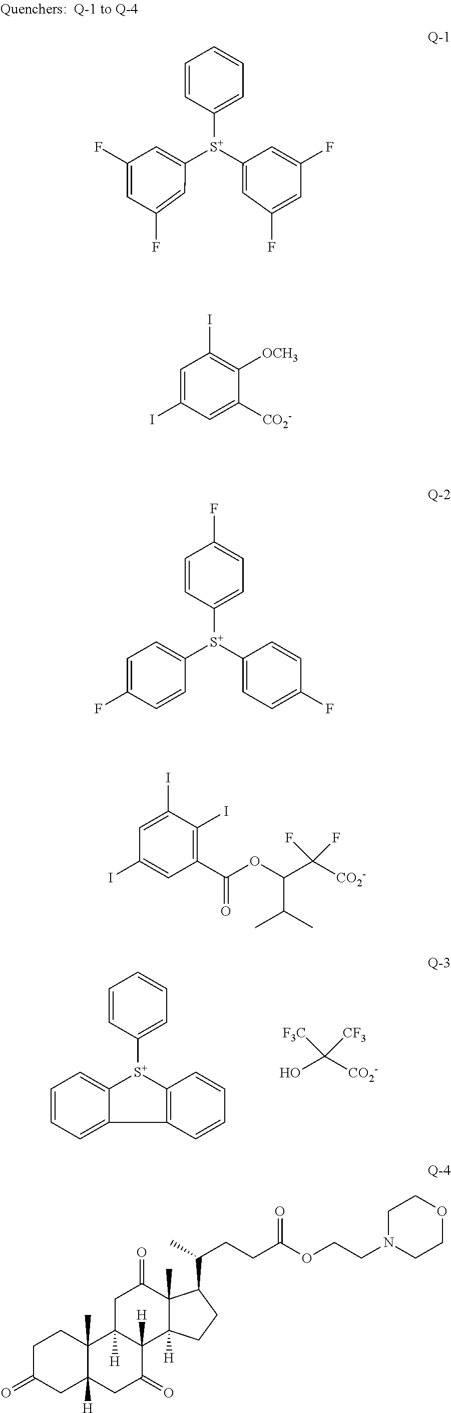

Surfactant A: 3-methyl-3-(2,2,2-trifluoroethoxymethyl) oxetane/tetrahydrofuran/2,2-dimethyl-1,3-propanediol copolymer (manufactured by OMNOVA)

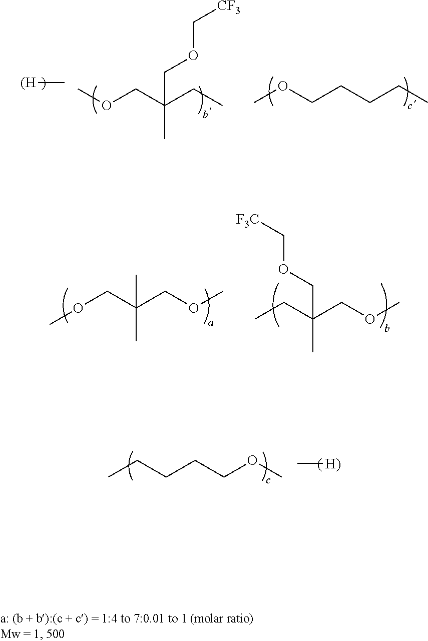

### [4] EUV Lithography Evaluation (1)

### Examples 4-1 to 4-26, Comparative Examples 4-1 to 4-18

Each chemically amplified resist composition (R-1 to R-26, CR-1 to CR-18) shown in Tables 3 and 4 was spin-coated onto an Si substrate on which a silicon-containing spin-on hard mask SHB-A940 (silicon content: 43% by mass) manufactured by Shin-Etsu Chemical Co., Ltd. had been formed to a thickness of 20 nm, and the substrate was pre-baked at 100° C. for 60 seconds using a hot plate to produce a resist film with a thickness of 50 nm. The resist film was subjected to an LS pattern exposure with a wafer dimension of 18 nm and a pitch of 36 nm using an EUV scanner NXE3400 (NA 0.33, σ 0.9/0.6, dipole illumination), manufactured by ASML, with the exposure amount and focus changed (exposure amount pitch: 1 mJ/cm², focus pitch: 0.020 μm), and after exposure, PEB was performed for 60 seconds at the temperatures shown in Tables 5 and 6. After that, paddle development was performed for 30 seconds using a 2.38% by mass TMAH aqueous solution, followed by rinsing with a surfactant-containing rinse material and spin drying to obtain a positive pattern.

The obtained LS pattern was observed with a CD-SEM (CG6300), manufactured by Hitachi High-Tech Corporation, and the sensitivity, the EL, the LWR, the depth of focus (DOF), and the collapse limit were evaluated according to the following methods. Furthermore, the development defects of the obtained LS patterns were evaluated. The results are shown in Tables 5 and 6.

### [Sensitivity Evaluation]

The optimal exposure amount Eop (mJ/cm²) for obtaining an LS pattern with a line width of 18 nm and a pitch of 36 nm was determined, and this was used as the sensitivity. The smaller this value, the higher the sensitivity.

### [EL Evaluation]

The EL (unit: %) was calculated from the exposure amount formed within the range of ±10% (16.2 to 19.8 nm) of the 18 nm space width in the LS pattern using the following formula. The higher this value, the better the performance.

$$EL ( \% ) = ( \vert {E}_{1} - {E}_{2} \vert / Eop ) \times 100$$

E₁: Optimal exposure amount for LS pattern with line width of 16.2 nm and pitch of 36 nm
E₂: Optimal exposure amount for LS pattern with line width of 19.8 nm and pitch of 36 nm
Eop: Optimal exposure amount for LS pattern with line width of 18 nm and pitch of 36 nm

### [LWR Evaluation]

The dimensions of the LS pattern obtained by irradiating with Eop were measured at 10 points along the longitudinal direction of the line, the standard deviation (a) was calculated and tripled, and the result (3σ) was used as the LWR. The smaller this value, the less roughness and the more uniform line width the pattern that can be obtained will have.

### [DOF Evaluation]

To evaluate the depth of focus, the focus range formed within ±10% (16.2 to 19.8 nm) of the 18 nm dimension in the LS pattern was obtained. The larger this value, the wider the depth of focus.

### [Line Pattern Collapse Limit Evaluation]

The line dimensions of the LS pattern at each exposure amount at the optimal focus were measured at 10 points in the longitudinal direction. The thinnest line dimension that could be obtained without collapse was used as the collapse limit dimension. The smaller this value, the better the collapse limit.

TABLE 5

| | | | Optimal | | | | |
|---|---|---|---|---|---|---|---|
| | | PEB | exposure | | | | Collapse |
| | Resist | temperature | amount | EL | LWR | DOF | limit |
| | composition | (° C.) | (mJ/cm²) | (%) | (nm) | (nm) | (nm) |
| Example 4-1 | R-1 | 95 | 33 | 18 | 2.3 | 120 | 10.9 |
| Example 4-2 | R-2 | 100 | 33 | 19 | 2.4 | 110 | 10.7 |
| Example 4-3 | R-3 | 100 | 32 | 17 | 2.5 | 120 | 10.6 |
| Example 4-4 | R-4 | 95 | 32 | 18 | 2.3 | 110 | 11.1 |
| Example 4-5 | R-5 | 105 | 33 | 17 | 2.4 | 100 | 11.2 |
| Example 4-6 | R-6 | 100 | 32 | 17 | 2.5 | 120 | 11.1 |
| Example 4-7 | R-7 | 95 | 34 | 18 | 2.5 | 110 | 11.2 |
| Example 4-8 | R-8 | 95 | 32 | 19 | 2.3 | 100 | 11.3 |
| Example 4-9 | R-9 | 100 | 33 | 17 | 2.4 | 110 | 11.2 |
| Example 4-10 | R-10 | 100 | 34 | 18 | 2.3 | 120 | 11.3 |
| Example 4-11 | R-11 | 100 | 35 | 19 | 2.3 | 120 | 11.3 |
| Example 4-12 | R-12 | 95 | 34 | 17 | 2.4 | 110 | 10.8 |
| Example 4-13 | R-13 | 105 | 33 | 18 | 2.5 | 120 | 10.6 |
| Example 4-14 | R-14 | 100 | 32 | 17 | 2.3 | 100 | 10.9 |
| Example 4-15 | R-15 | 95 | 33 | 17 | 2.4 | 110 | 10.7 |
| Example 4-16 | R-16 | 95 | 33 | 18 | 2.4 | 120 | 11.1 |
| Example 4-17 | R-17 | 100 | 35 | 17 | 2.5 | 110 | 11.2 |
| Example 4-18 | R-18 | 95 | 34 | 18 | 2.3 | 110 | 11.4 |
| Example 4-19 | R-19 | 95 | 33 | 18 | 2.4 | 120 | 11.3 |
| Example 4-20 | R-20 | 100 | 34 | 17 | 2.5 | 110 | 10.9 |
| Example 4-21 | R-21 | 100 | 35 | 19 | 2.6 | 110 | 10.7 |
| Example 4-22 | R-22 | 100 | 32 | 17 | 2.3 | 110 | 11.2 |
| Example 4-23 | R-23 | 95 | 34 | 18 | 2.4 | 110 | 11.3 |
| Example 4-24 | R-24 | 95 | 32 | 18 | 2.3 | 120 | 11.2 |
| Example 4-25 | R-25 | 100 | 35 | 17 | 2.5 | 110 | 11.1 |
| Example 4-26 | R-26 | 100 | 32 | 19 | 2.3 | 110 | 10.9 |

TABLE 6

| | | | Optimal | | | | |
|---|---|---|---|---|---|---|---|
| | | PEB | exposure | | | | Collapse |
| | Resist | temperature | amount | EL | LWR | DOF | limit |
| | composition | (° C.) | (mJ/cm²) | (%) | (nm) | (nm) | (nm) |
| Comparative Example 4-1 | CR-1 | 95 | 36 | 11 | 3.1 | 90 | 12.8 |
| Comparative Example 4-2 | CR-2 | 100 | 34 | 12 | 2.8 | 80 | 12.7 |
| Comparative Example 4-3 | CR-3 | 100 | 33 | 14 | 2.7 | 80 | 13.1 |
| Comparative Example 4-4 | CR-4 | 100 | 32 | 14 | 2.9 | 90 | 12.7 |
| Comparative Example 4-5 | CR-5 | 95 | 34 | 15 | 2.8 | 80 | 12.3 |
| Comparative Example 4-6 | CR-6 | 100 | 34 | 13 | 3.7 | 60 | 12.9 |
| Comparative Example 4-7 | CR-7 | 100 | 35 | 12 | 2.8 | 90 | 11.9 |
| Comparative Example 4-8 | CR-8 | 100 | 33 | 14 | 2.9 | 80 | 12.1 |
| Comparative Example 4-9 | CR-9 | 95 | 34 | 13 | 2.8 | 90 | 12.2 |
| Comparative Example 4-10 | CR-10 | 100 | 35 | 14 | 2.8 | 80 | 12.4 |
| Comparative Example 4-11 | CR-11 | 100 | 34 | 13 | 2.9 | 80 | 12.1 |
| Comparative Example 4-12 | CR-12 | 100 | 33 | 14 | 2.9 | 80 | 11.8 |
| Comparative Example 4-13 | CR-13 | 95 | 33 | 12 | 2.8 | 90 | 11.9 |
| Comparative Example 4-14 | CR-14 | 105 | 34 | 11 | 3.6 | 50 | 12.9 |
| Comparative Example 4-15 | CR-15 | 100 | 34 | 13 | 3.2 | 70 | 12.5 |
| Comparative Example 4-16 | CR-16 | 95 | 37 | 14 | 2.9 | 80 | 12.3 |
| Comparative Example 4-17 | CR-17 | 95 | 40 | 13 | 3.4 | 60 | 12.6 |
| Comparative Example 4-18 | CR-18 | 100 | 41 | 12 | 3.3 | 60 | 12.4 |

The results shown in Tables 5 and 6 show that the chemically amplified resist composition containing a polymer containing a repeating unit derived from a monomeric photo-acid generator consisting of an onium salt type monomer of the present invention has good sensitivity and is excellent in terms of EL, LWR, and DOF. Furthermore, it was confirmed that the value for the collapse limit was small, and that the pattern was resistant to collapse even in the formation of fine patterns. Therefore, it was shown that the chemically amplified resist composition of the present invention is suitable as a material for EUV lithography.

### [5] EUV Lithography Evaluation (2)

### Examples 5-1 to 5-26, Comparative Examples 5-1 to 5-18

Each chemically amplified resist composition (R-1 to R-26, CR-1 to CR-18) shown in Tables 3 and 4 was spin-coated onto an Si substrate on which a silicon-containing spin-on hard mask SHB-A940 (silicon content: 43% by mass) manufactured by Shin-Etsu Chemical Co., Ltd. had been formed to a thickness of 20 nm, and the substrate was pre-baked at 105° C. for 60 seconds using a hot plate to produce a resist film with a thickness of 50 nm. The resist film was exposed using an EUV scanner NXE3400 (NA: 0.33; σ 0.9/0.6; quadrupole illumination; on-wafer dimension pitch: 46 nm; +20% bias hole pattern mask), manufactured by ASML, PEB was performed for 60 seconds using a hot plate at the temperatures listed in Tables 7 and 8, and development was performed for 30 seconds with a 2.38% by mass TMAH aqueous solution to form a hole pattern with a dimension of 23 nm.

Using a CD-SEM (CG6300), manufactured by Hitachi High-Tech Corporation, the exposure amount when a hole dimension of 23 nm was formed was measured and this was used as the sensitivity, and the dimensions of 50 holes at this time were also measured, the standard deviation (σ) was calculated and tripled, and the result (3σ) was used as the CDU. The results are shown in Tables 7 and 8.

TABLE 7

| | | | Optimal | |
|---|---|---|---|---|
| | | PEB | exposure | |
| | Resist | temperature | amount | CDU |
| | composition | (° C.) | (mJ/cm²) | (nm) |
| Example 5-1 | R-1 | 95 | 24 | 2.2 |
| Example 5-2 | R-2 | 95 | 23 | 2.5 |
| Example 5-3 | R-3 | 90 | 24 | 2.2 |
| Example 5-4 | R-4 | 90 | 23 | 2.3 |
| Example 5-5 | R-5 | 90 | 23 | 2.4 |
| Example 5-6 | R-6 | 95 | 25 | 2.5 |
| Example 5-7 | R-7 | 95 | 24 | 2.4 |
| Example 5-8 | R-8 | 90 | 23 | 2.5 |
| Example 5-9 | R-9 | 95 | 24 | 2.4 |
| Example 5-10 | R-10 | 95 | 23 | 2.2 |
| Example 5-11 | R-11 | 95 | 25 | 2.4 |
| Example 5-12 | R-12 | 90 | 23 | 2.6 |
| Example 5-13 | R-13 | 90 | 24 | 2.4 |
| Example 5-14 | R-14 | 90 | 23 | 2.3 |
| Example 5-15 | R-15 | 90 | 25 | 2.4 |
| Example 5-16 | R-16 | 85 | 24 | 2.5 |
| Example 5-17 | R-17 | 95 | 24 | 2.4 |
| Example 5-18 | R-18 | 95 | 24 | 2.2 |
| Example 5-19 | R-19 | 90 | 23 | 2.5 |
| Example 5-20 | R-20 | 95 | 23 | 2.4 |
| Example 5-21 | R-21 | 95 | 25 | 2.4 |
| Example 5-22 | R-22 | 95 | 24 | 2.2 |
| Example 5-23 | R-23 | 95 | 23 | 2.4 |
| Example 5-24 | R-24 | 90 | 24 | 2.5 |
| Example 5-25 | R-25 | 95 | 22 | 2.4 |
| Example 5-26 | R-26 | 95 | 23 | 2.2 |

TABLE 8

| | | | Optimal | |
|---|---|---|---|---|
| | | PEB | exposure | |
| | Resist | temperature | amount | CDU |
| | composition | (° C.) | (mJ/cm²) | (nm) |
| Comparative | CR-1 | 95 | 29 | 3.1 |
| Example 5-1 | | | | |
| Comparative | CR-2 | 95 | 26 | 2.8 |
| Example 5-2 | | | | |
| Comparative | CR-3 | 95 | 25 | 2.7 |
| Example 5-3 | | | | |
| Comparative | CR-4 | 90 | 24 | 2.8 |
| Example 5-4 | | | | |
| Comparative | CR-5 | 90 | 24 | 2.8 |
| Example 5-5 | | | | |
| Comparative | CR-6 | 95 | 25 | 3.4 |
| Example 5-6 | | | | |
| Comparative | CR-7 | 90 | 24 | 2.8 |
| Example 5-7 | | | | |
| Comparative | CR-8 | 90 | 25 | 2.7 |
| Example 5-8 | | | | |
| Comparative | CR-9 | 90 | 23 | 2.8 |
| Example 5-9 | | | | |
| Comparative | CR-10 | 95 | 24 | 2.8 |
| Example 5-10 | | | | |
| Comparative | CR-11 | 95 | 25 | 2.7 |
| Example 5-11 | | | | |
| Comparative | CR-12 | 95 | 25 | 2.9 |
| Example 5-12 | | | | |
| Comparative | CR-13 | 85 | 24 | 2.8 |
| Example 5-13 | | | | |
| Comparative | CR-14 | 95 | 25 | 3.4 |
| Example 5-14 | | | | |
| Comparative | CR-15 | 95 | 25 | 3.2 |
| Example 5-15 | | | | |
| Comparative | CR-16 | 90 | 26 | 2.9 |
| Example 5-16 | | | | |
| Comparative | CR-17 | 95 | 32 | 3.3 |
| Example 5-17 | | | | |
| Comparative | CR-18 | 95 | 31 | 3.2 |
| Example 5-18 | | | | |

From the results shown in Tables 7 and 8, it was confirmed that the chemically amplified resist composition containing a polymer containing a repeating unit derived from a monomeric photo-acid generator consisting of an onium salt-type monomer of the present invention has good sensitivity and excellent CDU.

The present description includes the following embodiments.

[1]: An onium salt type monomer, wherein the onium salt type monomer is represented by the following general formula (A),

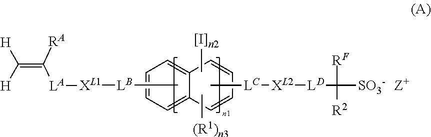

wherein “n1” represents 0 or 1; “n2” represents an integer from 1 to 4; “n3” represents an integer from 0 to 4; provided that when “n1” is 0, 1≤n2+n3≤4, and when “n1” is 1, 1≤n2+n3≤6;
R^{A} represents a hydrogen atom, a fluorine atom, a methyl group, or a trifluoromethyl group;
R¹ represents a halogen atom other than an iodine atom, a nitro group, a hydroxy group, a carboxy group, a hydrocarbyl group having 1 to 20 carbon atoms and optionally containing a heteroatom, a hydrocarbyloxy group having 1 to 20 carbon atoms and optionally containing a heteroatom, a hydrocarbylthio group having 1 to 20 carbon atoms and optionally containing a heteroatom, or a hydrocarbyloxycarbonyl group having 2 to 20 carbon atoms; when “n3” is 2, 3, or 4, each R¹ may be the same as or different from each other, and a plurality of R¹ may be bonded to each other to form a ring together with the carbon atoms to which they are bonded;
R² represents a hydrogen atom, or a hydrocarbyl group having 1 to 20 carbon atoms and optionally containing a heteroatom;
L^{A} represents a single bond, an ether bond, an ester bond, or a sulfonate ester bond;
L^{B}, L^{C}, and L^{D} each independently represent a single bond, an ether bond, an ester bond, a sulfonate ester bond, a carbonate bond, or a carbamate bond;
X^{L1} and X^{L2} each independently represent a single bond, or a hydrocarbylene group having 1 to 40 carbon atoms and optionally containing a heteroatom;
R^{F} represents a fluorine atom or a fluorinated saturated hydrocarbyl group having 1 to 6 carbon atoms; and
Z⁺ represents an onium cation.

[2]: The onium salt type monomer according to [1], wherein the onium salt type monomer is represented by the following general formula (A1),

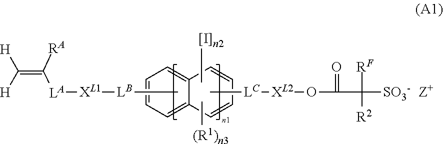

wherein R^{A}, R¹, R², R^{F}, L^{A}, L^{B}, L^{C}, X^{L1}, X^{L2}, “n1” to “n3”, and Z⁺ are as defined above.

[3]: The onium salt type monomer according to [2], wherein the onium salt type monomer represented by the general formula (A1) is represented by the following general formula (A2),

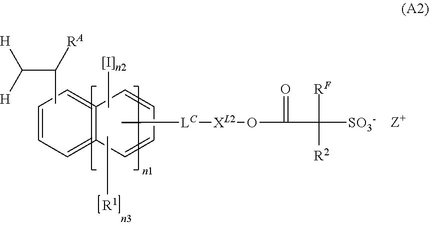

wherein R^{A}, R¹, R², R^{F}, L^{C}, X^{L2}, “n1” to “n3”, and are as defined above.

[4]: The onium salt type monomer according to any one of [1] to [3], wherein Z⁺ represents a sulfonium cation represented by the following general formula (Z-1) or an iodonium cation represented by the following general formula (Z-2),

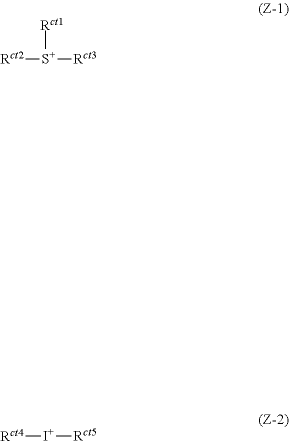

wherein R^{ct1} to R^{ct5} each independently represent a halogen atom, or a hydrocarbyl group having 1 to 30 carbon atoms and optionally containing a heteroatom; and also, R^{ct1} and R^{ct2} may be bonded to each other to form a ring together with the sulfur atom to which they are bonded.

[5]: A monomeric photo-acid generator, comprising the onium salt type monomer according to any one of [1] to [4].

[6]: A polymer, comprising a repeating unit derived from the monomeric photo-acid generator according to [5].

[7]: The polymer according to [6], further comprising at least one repeating unit selected from the group consisting of a repeating unit represented by the following general formula (a1), a repeating unit represented by the following general formula (a2), and a repeating unit represented by the following general formula (a3),

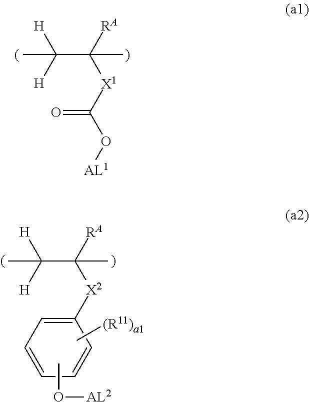

wherein “a1” is an integer from 0 to 4;
R^{A} each independently represents a hydrogen atom, a fluorine atom, a methyl group, or a trifluoromethyl group;
X¹ represents a single bond, a phenylene group, a naphthylene group, or *—C(═O)—O—X¹¹—, and the phenylene group or naphthylene group may be substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom; X¹¹ represents a saturated hydrocarbylene group having 1 to 10 carbon atoms, a phenylene group, or a naphthylene group, and the saturated hydrocarbylene group may contain a hydroxy group, an ether bond, an ester bond, or a lactone ring;
X² represents a single bond or *—C(═O)—O—;
“*” represents an attachment point to a main chain carbon atom;
R²¹ represents a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; when “a1” is 2 or more, each R²¹ may be the same as or different from each other; and
AL¹ and AL² each independently represent an acid-labile group,

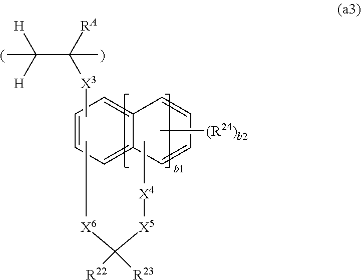

wherein “b1” represents 0 or 1; “b2” represents an integer from 0 to 3 when “b1” is 0, and represents an integer from 0 to 5 when “b1” is 1;
R^{A} represents a hydrogen atom, a fluorine atom, a methyl group, or a trifluoromethyl group;
X³ represents a single bond, *—C(═O)—O—, or *—C(═O)—NH—; “*” represents an attachment point to a main chain carbon atom;
X⁴ represents a single bond, an aliphatic hydrocarbylene group having 1 to 4 carbon atoms, a carbonyl group, a sulfonyl group, or a group obtained by combining these;
X⁵ and X⁶ each independently represent an oxygen atom or a sulfur atom; provided that X⁴ and X⁶ are bonded to adjacent carbon atoms of an aromatic ring;
R²² and R²³ each independently represent a hydrogen atom, or a hydrocarbyl group having 1 to 20 carbon atoms and optionally containing a heteroatom; also, R²² and R²³ may be bonded to each other to form a ring together with the carbon atom to which they are bonded;
R²⁴ represents a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, a hydrocarbylthio group having 1 to 20 carbon atoms and optionally containing a heteroatom, or —N(R^{24A}) (R^{24B}); R^{24A} and R^{24B} each independently represent a hydrogen atom or a hydrocarbyl group having 1 to 6 carbon atoms; and when “b2” is 2 or more, each R²⁴ may be the same as or different from each other, and a plurality of R²⁴ may be bonded to each other to form a ring together with the carbon atoms of the aromatic ring to which they are bonded.

[8]: The polymer according to [6] or [7], further comprising at least one repeating unit selected from the group consisting of a repeating unit represented by the following general formula (b1), and a repeating unit represented by the following general formula (b2),

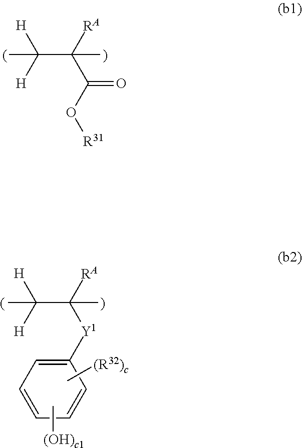

wherein “c1” is an integer from 1 to 4; “c2” represents an integer from 0 to 4; and 1≤c1+c2≤5;
R^{A} each independently represents a hydrogen atom, a fluorine atom, a methyl group, or a trifluoromethyl group;
Y¹ represents a single bond or *—C(═O)—O—; “*” represents an attachment point to a main chain carbon atom;
R³¹ represents a hydrogen atom, or a group having 1 to 20 carbon atoms and containing at least one structure selected from the group consisting of a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—);
R³² represents a halogen atom, a carboxy group, a nitro group, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and when “c2” represents 2 or more, each R³² may be the same as or different from each other.

[9]: A chemically amplified resist composition, comprising a base polymer (A) comprising the polymer according to any one of [6] to [8].

[10]: The chemically amplified resist composition according to [9], further comprising at least one selected from the group consisting of an organic solvent (B), a quencher (C), a photo-acid generator (D) other than the monomeric photo-acid generator, and a surfactant (E).

[11]: A patterning process, comprising the steps of: forming a resist film on a substrate using the chemically amplified resist composition according to [9] or [10]; exposing the resist film with a high-energy beam; and developing the exposed resist film using a developer.

[12]: The patterning process according to [11], wherein the high-energy beam is KrF excimer laser light, ArF excimer laser light, an electron beam, or extreme ultraviolet having a wavelength of 3 to 15 nm.

It should be noted that the present invention is not limited to the above-described embodiments. The embodiments are just examples, and any examples that have substantially the same features and demonstrate the same functions and effects as those in the technical concept disclosed in claims of the present invention are included in the technical scope of the present invention.

1. An onium salt type monomer, wherein the onium salt type monomer is represented by the following general formula (A), 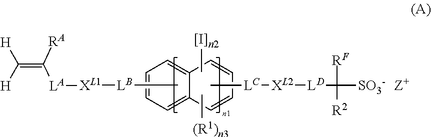
wherein “n1” represents 0 or 1; “n2” represents an integer from 1 to 4; “n3” represents an integer from 0 to 4; provided that when “n1” is 0, 1≤n2+n3≤4, and when “n1” is 1, 1≤n2+n3≤6;
R^{A} represents a hydrogen atom, a fluorine atom, a methyl group, or a trifluoromethyl group;
R¹ represents a halogen atom other than an iodine atom, a nitro group, a hydroxy group, a carboxy group, a hydrocarbyl group having 1 to 20 carbon atoms and optionally containing a heteroatom, a hydrocarbyloxy group having 1 to 20 carbon atoms and optionally containing a heteroatom, a hydrocarbylthio group having 1 to 20 carbon atoms and optionally containing a heteroatom, or a hydrocarbyloxycarbonyl group having 2 to 20 carbon atoms; when “n3” is 2, 3, or 4, each R¹ may be the same as or different from each other, and a plurality of R¹ may be bonded to each other to form a ring together with the carbon atoms to which they are bonded;
R² represents a hydrogen atom, or a hydrocarbyl group having 1 to 20 carbon atoms and optionally containing a heteroatom;
L^{A} represents a single bond, an ether bond, an ester bond, or a sulfonate ester bond;
L^{B}, L^{C}, and L^{D} each independently represent a single bond, an ether bond, an ester bond, a sulfonate ester bond, a carbonate bond, or a carbamate bond;
X^{L1} and X^{L2} each independently represent a single bond, or a hydrocarbylene group having 1 to 40 carbon atoms and optionally containing a heteroatom;
R^{F} represents a fluorine atom or a fluorinated saturated hydrocarbyl group having 1 to 6 carbon atoms; and
Z⁺ represents an onium cation.

2. The onium salt type monomer according to claim 1, wherein the onium salt type monomer is represented by the following general formula (A1), 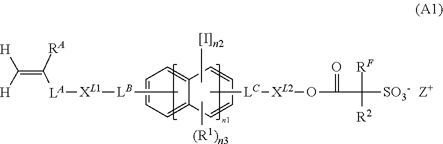
wherein R^{A}, R¹, R², R^{F}, L^{A}, L^{B}, L^{C}, X^{L1}, X^{L2}, “n1” to “n3”, and Z⁺ are as defined above.

3. The onium salt type monomer according to claim 2, wherein the onium salt type monomer represented by the general formula (A1) is represented by the following general formula (A2), 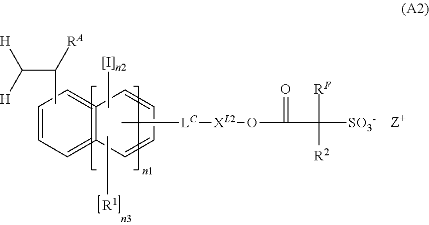
wherein R^{A}, R¹, R², R^{F}, L^{C}, X^{L2}, “n1” to “n3”, and Z⁺ are as defined above.

4. The onium salt type monomer according to claim 1, wherein Z⁺ represents a sulfonium cation represented by the following general formula (Z-1) or an iodonium cation represented by the following general formula (Z-2), 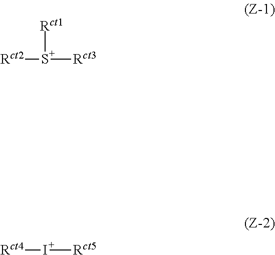
wherein R^{ct1} to R^{ct5} each independently represent a halogen atom, or a hydrocarbyl group having 1 to 30 carbon atoms and optionally containing a heteroatom; and also, R^{ct1} and R^{ct2} may be bonded to each other to form a ring together with the sulfur atom to which they are bonded.

5. A monomeric photo-acid generator, comprising the onium salt type monomer according to claim 1.

6. A polymer, comprising a repeating unit derived from the monomeric photo-acid generator according to claim 5.

7. The polymer according to claim 6, further comprising at least one repeating unit selected from the group consisting of a repeating unit represented by the following general formula (a1), a repeating unit represented by the following general formula (a2), and a repeating unit represented by the following general formula (a3), 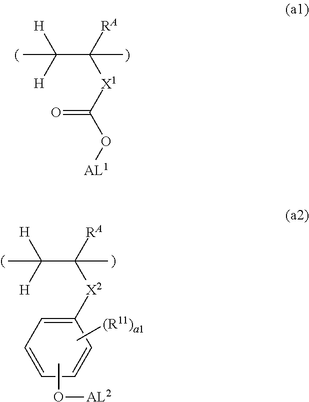
wherein “a1” is an integer from 0 to 4;
R^{A} each independently represents a hydrogen atom, a fluorine atom, a methyl group, or a trifluoromethyl group;
X¹ represents a single bond, a phenylene group, a naphthylene group, or *—C(═O)—O—X¹¹—, and the phenylene group or naphthylene group may be substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom; X¹¹ represents a saturated hydrocarbylene group having 1 to 10 carbon atoms, a phenylene group, or a naphthylene group, and the saturated hydrocarbylene group may contain a hydroxy group, an ether bond, an ester bond, or a lactone ring;
X² represents a single bond or *—C(═O)—O—;
“*” represents an attachment point to a main chain carbon atom;
R²¹ represents a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; when “a1” is 2 or more, each R²¹ may be the same as or different from each other; and
AL¹ and AL² each independently represent an acid-labile group, 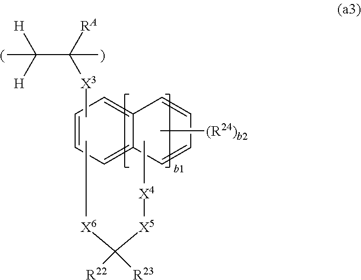
wherein “b1” represents 0 or 1; “b2” represents an integer from 0 to 3 when “b1” is 0, and represents an integer from 0 to 5 when “b1” is 1;
R^{A} represents a hydrogen atom, a fluorine atom, a methyl group, or a trifluoromethyl group;
X³ represents a single bond, *—C(═O)—O—, or *—C(═O)—NH—; “*” represents an attachment point to a main chain carbon atom;
X⁴ represents a single bond, an aliphatic hydrocarbylene group having 1 to 4 carbon atoms, a carbonyl group, a sulfonyl group, or a group obtained by combining these;
X⁵ and X⁶ each independently represent an oxygen atom or a sulfur atom; provided that X⁴ and X⁶ are bonded to adjacent carbon atoms of an aromatic ring;
R²² and R²³ each independently represent a hydrogen atom, or a hydrocarbyl group having 1 to 20 carbon atoms and optionally containing a heteroatom; also, R²² and R²³ may be bonded to each other to form a ring together with the carbon atom to which they are bonded;
R²⁴ represents a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, a hydrocarbylthio group having 1 to 20 carbon atoms and optionally containing a heteroatom, or —N(R^{24A}) (R^{24B}); R^{24A} and R^{24B} each independently represent a hydrogen atom or a hydrocarbyl group having 1 to 6 carbon atoms; and when “b2” is 2 or more, each R²⁴ may be the same as or different from each other, and a plurality of R²⁴ may be bonded to each other to form a ring together with the carbon atoms of the aromatic ring to which they are bonded.

8. The polymer according to claim 6, further comprising at least one repeating unit selected from the group consisting of a repeating unit represented by the following general formula (b1), and a repeating unit represented by the following general formula (b2), 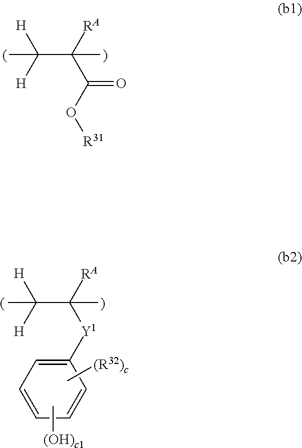
wherein “c1” is an integer from 1 to 4; “c2” represents an integer from 0 to 4; and 1≤c1+c2≤5;
R^{A} each independently represents a hydrogen atom, a fluorine atom, a methyl group, or a trifluoromethyl group;
Y¹ represents a single bond or *—C(═O)—O—; “*” represents an attachment point to a main chain carbon atom;
R³¹ represents a hydrogen atom, or a group having 1 to 20 carbon atoms and containing at least one structure selected from the group consisting of a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—);
R³² represents a halogen atom, a carboxy group, a nitro group, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and when “c2” represents 2 or more, each R³² may be the same as or different from each other.

9. A chemically amplified resist composition, comprising a base polymer (A) comprising the polymer according to claim 6.

10. The chemically amplified resist composition according to claim 9, further comprising at least one selected from the group consisting of an organic solvent (B), a quencher (C), a photo-acid generator (D) other than the monomeric photo-acid generator, and a surfactant (E).

11. A patterning process, comprising the steps of: forming a resist film on a substrate using the chemically amplified resist composition according to claim 9; exposing the resist film with a high-energy beam; and developing the exposed resist film using a developer.

12. The patterning process according to claim 11, wherein the high-energy beam is KrF excimer laser light, ArF excimer laser light, an electron beam, or extreme ultraviolet having a wavelength of 3 to 15 nm.
